(19) United States
(12) Patent Application Publication
(10) Pub. No.: US 2022/0370591 A1
(43) Pub. Date: Nov. 24, 2022

(54) COMPOSITIONS COMPRISING NUCLEIC ACIDS ENCODING STRUCTURAL TRIMERS AND METHODS OF USING THE SAME

(71) Applicant: THE WISTAR INSTITUTE OF ANATOMY & BIOLOGY, Philadelphia, PA (US)

(72) Inventors: Megan WISE, Plymouth Meeting, PA (US); Daniel W. KULP, Moorestown, NJ (US); David B. WEINER, Merion, PA (US)

(21) Appl. No.: 17/601,412

(22) PCT Filed: Apr. 6, 2020
(86) PCT No.: PCT/US20/26948

§ 371 Date: Oct. 4, 2021

Related U.S. Application Data
(60) Provisional application No. 62/829,629, filed on Apr. 4, 2019.

(51) Int. Cl. A61K 39/12; A61P 31/18; A61K 47/00

(52) U.S. Cl. CPC A61K 39/12; A61P 31/18; A61K 47/00; A61K 2039/53

(57) ABSTRACT

Disclosed are compositions comprising an expressible nucleic acid sequence comprising a first nucleic acid sequence comprising a leader sequence or a pharmaceutically acceptable salt thereof; and a second nucleic acid sequence comprising a sequence that encodes a trimer of a retroviral envelope or a pharmaceutically acceptable salt thereof. In some embodiments, the expressible nucleic acid sequence further comprises a nucleic acid sequence encoding at least one viral antigen or a pharmaceutically acceptable salt thereof. In some embodiments, the expressible nucleic acid sequence further comprises at least one nucleic acid sequence encoding a linker. Also disclosed are pharmaceutical compositions comprising these compositions and methods of using the disclosed compositions.

### CROSS-REFERENCE TO RELATED APPLICATIONS

This application claims priority to U.S. Provisional Application No. 62/829,629 filed on Apr. 4, 2019, which is incorporated by reference in its entirety.

### STATEMENT REGARDING FEDERALLY SPONSORED RESEARCH

The embodiments disclosed herein were made with government support under U19 A1109646-04 awarded by the National Institutes of Health. The government has certain rights in the embodiments.

### BACKGROUND

Despite extensive research and efforts, an efficacious HIV vaccine still eludes scientists. Two hurdles in HIV-1 vaccine development include the diversity of the HIV surface protein, Envelope, as well as the structure of this protein [1, 2]. Many vaccines have included subunits of Env which have generated significant binding antibodies but lack any effector functions, specifically neutralizing the HIV-1 virus [3, 4]. Recent advances in structural engineering and imaging have allowed for the development of a very limited number of properly folded native like HIV trimers [5-7]. However these are slow to develop and exceptionally costly to move to clinical testing. Furthermore, only a small number have been tested due to these issues and even the functional trimers lack the breadth necessary for broad protection against HIV. A new method for developing such complicated molecules directly in vivo would be game changing for this approach and would allow simple complex formulations that can be delivered as groups and provide broader immune protection. In addition, current recombinant methods for Trimer protein development cannot induce CD8 T cells but are limited to the induction of CD4 T helper responses, as well as antibody responses. Therefore, the current method lacks a critical immune component thought to be important for protection from HIV infection as well as for viral clearance. Presented herein is a demonstration that by designing synthetic DNA's that can fold in vivo to give complex structures native like trimers can be produced that yield improved T cell and antibody responses directly in living mammals, thus greatly advancing the vaccine field.

Through protein engineering in the laboratory there have been various forms of stabilized native like trimers which incorporate different amino acid mutations, modifications and truncations which allow for the proper folding and production of these trimers [6, 8][6, 8-11]. However, these proteins can be difficult to produce and purify, leading to lengthy manufacturing time and cost, which limits the application of this approach and in fact makes it a daunting challenge as a vaccine approach [12, 13]. Technologies that would allow de novo trimer formation in the host could potentially surpass the synthesis and purification steps required of recombinant trimer production. This would facility the rapid translation and iteration of various HIV-1 Env trimer strains both pre-clinically and in the clinic allowing for more diverse and protective collections of immunogens with improved deliverability. With the recent significant improvement in immunogenicity, the induction of potent specific humoral responses in animals and in the clinic, coupled with improved designs for complex synthetic molecule that require folding in vivo, the synthetic DNA vaccination platform represents an important tool for next generation design of viral antigens where in vivo folding of their antigens are important for immune function. Importantly, the work in the plasmid encoded synthetic DNA space has recently improved its ability to encode highly complex folded structures in vivo and have been described as highly functional and potent synthetic DNA encoded monoclonal antibodies launched directly in vivo [19-24].

### SUMMARY OF EMBODIMENTS

Described herein is an in vivo molecule self-assembly for, in this case, HIV Envelope trimers through the use of advanced synthetic nucleic acid electroporation technology to rapidly design, encode, fold, express and or secrete various forms of HIV-1 native like trimers including long designed forms in vivo. Synthetic DNA encoded trimers can fold tightly and assume relevant conformations important for maintaining Envelope shape in vivo. These in vivo produced immunogens serve to induce autologous neutralizing antibodies and strong antigen specific T cell responses with robust CD4 helper responses in small animal models. This combination has not been previously achievable in a single platform. These responses can be further tailored to express novel trimer structures to focus the immune response in important ways by encoding modifications to the DNA sequence on the nucleotide level resulting in a final molecule that assembles in vivo capable of preventing or eliminating non-desired off-target antibody responses. This translates to many advantages for vaccine development including the ability to quickly produce or modify functional HIV Env trimers using simple DNA plasmids that allow for enhanced vaccine designs including for complex mixtures of trimers. These can be simply formatted as DNA as stable vaccine formulations in a simple, rapid deliverable and cost saving form for product development.

There are significant limitations in administering therapeutically effective amounts of protein vaccines to subjects, including ensuring that appropriate levels of the vaccine become exposed to antigen presenting cells and that the magnitude of any immune response is sufficient after administration of a single bolus dose. Furthermore, protein vaccines are difficult to store for relatively long periods of time because of protein instability issues. Synthetic designed DNA as immunogens represents an alternative vaccination technique that initially was limited in potency due to poor in vivo delivery and uptake. Encapsulation in compounds of older DNA was studied in the area of gene therapy and gene delivery but is expensive due to high doses needed, and poorly expressing, resulting in weak immunity and no functional immunity in vivo. To date, such approaches have not been reported to allow for reproducible complex molecule assembly in vivo for either biologic or vaccine production. Improved delivery technologies for synthetic DNA delivery is important in this regard.

To address these additional limitations and the limitations associated with biologic manufacturing of viral nanoparticles, the present disclosure relates to designing optimized nucleic acid sequences that can encode naturally self-assembling nanoparticles, that are not dependent on chemical formulations, as well as designed large antigen fragments and compositions comprising the same. In some embodiments, the disclosure relates to compositions comprising an expressible nucleic acid sequence comprising a first nucleic acid sequence comprising a leader sequence or a pharmaceutically acceptable salt thereof; and a second nucleic acid sequence comprising a sequence that encodes a self-assembling polypeptide or a pharmaceutically acceptable salt thereof. In some embodiments, the expressible nucleic acid sequence further encodes a third nucleic acid sequence that is a viral antigen. When the nucleic acid sequence is adminstered to a subject in the context of a method of treatment or prevention of the viral infection, antigen presenting cells can be transduced or transfected with the nucleic acid sequences disclosed herein to produce conformationally stable trimer polypeptides of pathogenic virus that more adequately elicit antigen-specific immune responses against the virus.

Disclosed are compositions comprising a nucleic acid sequence comprising at least one expressible nucleic acid sequence. In some embodiments, the composition comprises at least one, two, three, or more expressible nucleic acid sequences, wherein at least one of the expressible nucleic acid sequence comprises:
(i) one or a combination of nucleic acid sequences chosen from: SEQ ID NO: 53, SEQ ID NO: 54, SEQ ID NO: 56, SEQ ID NO: 57. SEQ ID NO: 59, SEQ ID NO: 60, SEQ ID NO: 62, SEQ ID NO: 63, SEQ ID NO: 78. SEQ ID NO: 79, SEQ ID NO: 81, SEQ ID NO: 82, SEQ ID NO: 84, SEQ ID NO: 85, SEQ ID NO: 87, SEQ ID NO: 88, SEQ ID NO: 90, SEQ ID NO: 91, SEQ ID NO:106, 107 SEQ ID NO: 109, SEQ ID NO: 110, SEQ ID NO: 112, SEQ ID NO: 113, SEQ ID NO: 115, SEQ ID NO: 116, SEQ ID NO: 118, SEQ ID NO: 119, SEQ ID NO: 121, SEQ ID NO: 122, SEQ ID NO: 124, SEQ ID NO: 125, SEQ ID NO: 127, SEQ ID NO: 128, SEQ ID NO: 130, SEQ ID NO: 131; and/or
(ii) one or a combination of nucleic acid sequences wherein the at least one nucleic acid sequence comprises at least 70% sequence identity to a sequence identified as including: AD8, CPG9.2, 001428, TRO11, X2278, 398F1, 246F3, CE0217, CE1176, 25710, BJOX2000, CHI19, X1632, CNE8, CNE55, or 001428; and/or
(iii) one or a combination of nucleic acid sequences that encode an amino acid sequence chosen from: SEQ ID NO: 55, SEQ ID NO: 58, SEQ ID NO: 61, SEQ ID NO: 49, SEQ ID NO: 50, SEQ ID NO: 51, SEQ ID NO: 52, SEQ ID NO: 53, SEQ ID NO: 64, SEQ ID NO: 80, SEQ ID NO: 83, SEQ ID NO: 86, SEQ ID NO:89, SEQ ID NO: 92, SEQ ID NO: 93, SEQ ID NO: 94, SEQ ID NO: 108, SEQ ID NO: 111, SEQ ID NO: 114, SEQ ID NO: 117, SEQ ID NO: 120, SEQ ID NO: 123, SEQ ID NO: 93, SEQ ID NO: 126, SEQ ID NO: 129, SEQ ID NO: 132; and/or
(iv) one or a combination of nucleic acid sequences that encode at least one amino acid sequences comprising at least 70% sequence identity to a sequence identified as including: AD8, CPG9.2, 001428, TRO11, X2278, 398F1, 246F3, CE0217, CE1176, 25710, BJOX2000, CHI19, X1632, CNE8, CNE55, or 001428. Disclosed are compositions comprising an expressible nucleic acid sequence comprising a first nucleic acid sequence comprising a soluble retroviral trimer or a pharmaceutically acceptable salt thereof.

Disclosed are compositions comprising an expressible nucleic acid sequence comprising: a first nucleic acid sequence comprising at least 70% sequence identity to a nucleotide sequence encoding a soluble polypeptide monomer of or trimer of human immunodeficiency virus-1 (HIV-1) ENV; and a regulatory sequence operably linked to the first nucleotide sequence. Disclosed are pharmaceutical compositions comprising any of the compositions disclosed herein and a pharmaceutically acceptable carrier. In some embodiments, if a monomer is encoded it is a monomer capable of forming a trimer upon expression within a cell. In some embodiments, the expressible nucleic acid sequence further comprises a nucleic acid sequence encoding at least one viral antigen or a pharmaceutically acceptable salt thereof. In some embodiments, the expressible nucleic acid sequence further comprises at least one nucleic acid sequence encoding a linker. The disclosure also relates to pharmaceutical compositions comprising any one or more of the disclosed compositions and a pharmaceutically acceptable carrier.

Disclosed are methods of vaccinating a subject comprising administering a therapeutically effective amount of any of the disclosed pharmaceutical compositions to the subject. The disclosure relates to methods of inducing an immune response in a subject comprising administering to the subject any of the disclosed pharmaceutical compositions.

Disclosed are methods of neutralizing one or a plurality of viruses in a subject comprising administering to the subject any of the disclosed pharmaceutical compositions.

Disclosed are methods of stimulating a therapeutically effective antigen-specific immune response against a virus in a mammal infected with the virus comprising administering any of the disclosed pharmaceutical compositions. Disclosed are methods of inducing expression of a self-assembling vaccine in a subject comprising administering any of the disclosed pharmaceutical compositions.

Disclosed are vaccines comprising a first amino acid sequence comprising at least 70% sequence identity to a leader sequence; and/or a second amino acid sequence comprising at least 70% sequence identity to a linker sequence.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

Disclosed are methods of immunizing a subject in need thereof comprising administering a therapeutically effective amount of any of the disclosed pharmaceutical compositions to the subject. In some embodiments, the immunization is induced against HIV infection.

Also disclosed are methods of eliciting an antigen-specific immune response against a trimer in a subject in need thereof comprising administering a therapeutically effective amount of any of the disclosed pharmaceutical compositions to the subject. In some embodiments, the trimer is an HIV trimer.

In some embodiments, the administering in the disclosed methods is accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof. In some embodiments, the therapeutically effective dose in the disclosed methods is from about 1 to about 30 micrograms of expressible nucleic acid sequence. In some embodiments, the methods are free of activating any mannose-binding lectin or complement process. In some embodiments, the subject is a human. In some embodiments, the therapeutically effective dose in the disclosed methods is from about 0.001 micrograms of composition per kilogram of the subject to about 0.050 micrograms per kilogram of the subject. In some embodiments, any of the disclosed methods can be used in combination with retrovirals.

The disclosure relates to nucleic acid sequences that encode a retroviral antigen that are free of a transmembrane domain. In some embodiments, the retroviral antigen is the envelope glycoprotein gp120 of the HIV. In some embodiments, the retroviral antigen is free of the HIV-1 transmembrane domain gp41.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A, 1B, 1C, and 1D show DNA vs protein immunization and the superior T cell responses with DNA. A) schematic diagram of immunization schedule. Mice were immunized with 25 ug of pMD39-OPT DNA with IM EP-CELLECTRA 3P or 25 ug of protein MD39 formulated in RIBI and delivered to two sites SubQ. Each mouse received 3 immunizations at 3 week intervals. T cell responses were determined 2 weeks after final immunization using overlapping peptides for the WT BG505 Envelope virus sequence. B) IFNy ELISpots using BG505 WT Env peptides. DNA immunize mice have immune responses to the entire antigen. D) these T cell responses are to both CD4 and CD8 T cells. C & E) These T cells are polyfunctional and express multiple cytokines. DNA induces stronger T cell responses compared to protein using multiple different measures including IFN-y ELISpots and ICS. Both CD4 and CD8 were induced by DNA.

FIGS. 2A, 2B, 2C, and 2D show DNA vs protein immunization and similar binding titer responses. A) schematic diagram of immunization schedule. The dose used was 25 ug of pMD39-Opt; 25 ug of protein MD39; formulated in RIBI and delivered to two sites. B) The humoral responses for the mice were determined 2 weeks post each vaccinations. DNA is able to induce binding titers to trimeric HIV Env slightly higher than protein only immunizations. C) Post final immunization, there is not a significant difference between the two groups. D) It has been reported that mice cannot induce neutralizing titers to BG505 Tier 2 virus. However, using the same antigen, in rabbits and NHPs, autologous Tier 2, narrow neutralization titers were induced with protein. However, serum from mice immunized with DNA encoded trimers were able to induce autologous tier 2 neutralizing titers in 3 out of 10 mice. The serum was also tested against MLV to ensure there was not non-specific neutralization.

FIGS. 3A, 3B, 3C, 3D, and 3E show increasing the interval between immunizations improved cellular responses. A) schematic diagram of immunization schedule. Mice were immunized with 25 ug of pMD39_Opt DNA with IM-EP Cellectra 3P 3 times at either 0, 3, 6 or 0, 3, 16 weeks and euthanized the mice two weeks post final immunizations. B) IFNy ELISpots using BG505 WT Env peptides. Mice immunized at the longer interval had increase IFNy SFU. C) these T cell responses are to both CD4 and CD8 T cells. D & E) These T cells are polyfunctional and express multiple cytokines. The shorter immunization schedule induce better CD8 T cells where as the longer immunization had better CD4 T cell responses.

FIGS. 4A, 4B, 4C, 4D, and 4E shows increasing the interval between immunizations results in similar binding titers. A) schematic diagram of immunization schedule. 25 ug of plasmid DNA+IM-EP was used as the dose. B) and C) Binding to HIV Env Trimer over time. Observe good durability of antibody responses between the second and pre third immunizations. D) Binding antibodies to trimeric Env post final immunization. There is no difference in binding titers between the long vs short immunization schedule. E) Binding titers to monomers post final immunization—also similar between the two schedule.

FIGS. 5A, and 5B show increasing the interval between immunizations resulted in improved functional (neutralizing) antibodies. A) Even though binding titers were the same between the two groups, neutralization titers against autologous antibodies were stronger with the longer immunization. There were 7 out of 10 mice that induced autologous BG505 neutralization titers compared to 3 out of 10 with the short immunizations. B) The table shows the titers for each mouse as well as no neutralization with MLV.

FIGS. 6A, 6B, and 6C show similar trimer binding antibodies with soluble vs membrane bound trimers. All trimers were RNA and codon optimized and cloned into modified pVax 1 backbone with an IgE leader sequence added to the beginning of the construct. Modifications were made to the plasmid insert to tailor the vaccine induced responses A) schematic diagram of immunization schedule. Dosage is 25 ug of SynDNA+IM-EP CELLECTRA-3P; B) Trimer binding titer; C) GP120 monomer binding titer.

FIGS. 7A, 7B, and 7C show the strongest T cell responses are observed with soluble constructs. A) schematic diagram of immunization schedule. The dose is 25 ug of SynDNA+IM-EP CELLECTRA-3P. B) IFNy ELISpots using BG505 WT Env peptides. Mice immunized at the longer interval had increase IFNy SFU. C) these T cell responses are to both CD4 and CD8 T cells.

FIGS. 8A, 8B, and 8C show SynDNA trimers lower antibodies binding to V3 loop compared to controls. A) Full length V3; B) End V3; C) Tip V3. The exposure of these peptides decreases moving from A-C. There were no responses to scramble peptides. This supports that these antigens are being properly folded. DNA encoded structural immunogens decreases off target V3 binding antibody responses compared to GP120 foldon.

FIG. 9A show DNA encoded modifications limit bottom binding antibodies. A) Competition ELISA results using the bottom binding antibody 12N. On pMD39_opt the bottom of the trimer is exposed. Normally on the virus, this region is linked to the transmembrane domain and tethered to the virion. To prevent this exposure, glycans or linkers can be added. These modifications were tested to determine if they could decrease the bottom reactivity using a monoclonal that will bind to MD39 trimer (12N). Using a competition ELISA a significant decrease in the amount of antibodies that bind to the bottom of MD39 trimers with either glycans, linkers or forcing the antigen to be tethered to the membrane was observed. Different modifications can be encoded in DNA and can translate to in vivo immune responses. Additionally it is an indirect demonstration that glycan sites can be encoded and glycosylation events obtained.

FIG. 10 shows soluble SynDNA trimers induce better autologous (Tier 2) neutralizing antibody titers compared to other DNA encoded immunogens. The soluble antigens induce between 60-70% of autologous neutralizing antibody titers compared to 10-50% with the membrane bound antigens. There was no neutralization with MLV control virus. The graph represents a combination of two separate experiments.

FIGS. 11A, 11B show DNA induced NAb responses in mice do not target the 241/289 glycan hole but do target the T65n/C3 region of the Env. A) There is a monoclonal antibody which binds to the epitope which is dominant in rabbits immunized with a similar protein antigen, 11A. This antibody binds to a hole in the glycans on HIV Env at the 241 position. A competition ELISA was used to determine if the serum is binding to this epitope. Serum from mice immunized at wk 0, 3, 16 (wk 18 serum was used) for the competition with 11A. There was no competition with 11A from the mouse serum. B) To map where the serum was neutralizing, pseudotype viruses with various point mutations of known neutralization regions were used. Two groups of serum were used, those neutralizing BG505 autologous viruses (neutralizers) vs those that did not induce titers (non-neutralizers). There was no change in neutralization titers when the S241N mutations was made to the virus but here was a significant drop in neutralization when the T465N mutation was made. Thus, neutralizing antibodies are binding to the T465/C3 region of BG505. Furthermore, the maternal strain (MG505) which was the transmitting virus into the baby girl (BG505) for which this initial Env sequence was isolated, is closely related (17AA differences). One of these is in the region previously observed in NHPs (1396N). This could explain why MG505 is not neutralized by the mouse serum. MLV shown as a control.

FIG. 12A, 12B, 12C shows a rabbit study with SynDNA SOSIP Trimers immunizations. A) Diagramed of rabbit immunization schedule. Four different immunogens into rabbits, pOpt MD39, pOpt MD39_Glycan, pOpt_TS1, pOpt_TS1_PDGFR. Rabbits were immunized with either 1-2 mg of DNA based on the molar amount delivered to two sites ID with CELLECTRA 3P. Rabbits were immunized at week 0, 4, 12, 20. B) Binding to trimer over time; C) Binding to trimer week 14 (post 3^{rd} boost). Trimer specific antibody responses were detected with complete seroconversion post second immunization. These responses were slightly higher with MD39 compared to the other DNA encoded immunogens.

FIG. 13 shows an example of early titers against autologous BG505 T332N virus—First Tier 2 Neuts with SynDNA alone. Some neutralization titers were observed post third immunization against autologous viruses with boost following the forth immunization. There was limited to no non-specific neutralizing titers.

FIGS. 14A, 14B, and 14C shows a immunogenicity of selected synDNA trimers in a larger animal, non-human primate. A) Diagram of immunizations. NHPS (non-human primates) were immunized with 2 mgs of DNA delivered to two sites ID with CELLECTRA 3P. NHPS were immunized at weeks 0, 4, 12, and 20 with either pOpt MD39_Glycan or pOpt TS_1. B) IFNy ELISpots over time after stimulation with WT BG505 overlapping peptides. B) Week 14 individual NHPs. T cells responses were observed over background post 1 dose which are further expanded post dose 2 and 3. Most NHPS are responses to all parts of the antigen at week 6 and expand by week 14.

FIGS. 15A, 15B and 15C show humoral responses induced in NHP over time with synDNA encoded trimer immunogens. A) Trimer binding antibodies over time. Complete seroconversion is observed after the 2^{nd} dose. B) Binding titers to gp120 monomer over time. C) Comparison between the two groups binding titers to trimer vs gp120 monomer at week 14.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting. It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a nucleic acid sequence” includes a plurality of such sequences, reference to “the nucleic acid sequence” is a reference to one or more nucleic acid sequences and equivalents thereof known to those skilled in the art, and so forth.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” in context of an immunotherapy refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, indirect or direct ligation of cell surface moieties, even in the absence of a direct signal transduction event, may result in the reorganization of cytoskeletal structures, or in the coalescing of cell surface moieties, each of which could serve to enhance, modify, or alter subsequent cellular responses. As used herein, the terms “activating CD8+ T cells” or “CD8+ T cell activation” refer to a process (e.g., a signaling event) causing or resulting in one or more cellular responses of a CD8+ T cell (CTL), selected from: proliferation, differentiation, cytokine secretion, cytotoxic effector molecule release, cytotoxic activity, and expression of activation markers. As used herein, an “activated CD8+ T cell” refers to a CD8+ T cell that has received an activating signal, and thus demonstrates one or more cellular responses, selected from proliferation, differentiation, cytokine secretion, cytotoxic effector molecule release, cytotoxic activity, and expression of activation markers. Suitable assays to measure CD8+ T cell activation are known in the art and are described herein.

The term “combination therapy” as used herein is meant to refer to administration of one or more therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dose having a fixed ratio of each therapeutic agent or in multiple, individual doses for each of the therapeutic agents. For example, one combination of the present invention may comprise a pooled sample of one or more nucleic acid molecules comprising one or a plurality of expressible nucleic acid sequences and an adjuvant and/or an anti-viral agent administered at the same or different times. In some embodiments, the pharmaceutical composition of the disclosure can be formulated as a single, co-formulated pharmaceutical composition comprising one or more nucleic acid molecules comprising one or a plurality of expressible nucleic acid sequences and one or more adjuvants and/or one or more anti-viral agents. As another example, a combination of the present disclosure (e.g., DNA vaccines and anti-viral agent) may be formulated as separate pharmaceutical compositions that can be administered at the same or different time. As used herein, the term “simultaneously” is meant to refer to administration of one or more agents at the same time. For example, in certain embodiments, antiviral vaccine or immunogenic composition and antiviral agents are administered simultaneously). Simultaneously includes administration contemporaneously, that is during the same period of time. In certain embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, subcutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered intramuscularly only. The components may be administered in any therapeutically effective sequence. A “combination” embraces groups of compounds or non-small chemical compound therapies useful as part of a combination therapy.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain about 99%, 98%, 97%, 96%, 95%, 94%, 93%, 92%, 91%, or 90% sequence identity to the wild-type human sequence upon which the sequence is derived. In some embodiments, the functional fragment may retain about 85%, 80%, 75%, 70%, 65%, or 60% sequence identity to the wild-type sequence upon which the sequence is derived. By “fragment” is meant a portion of a polypeptide or nucleic acid molecule. This portion contains, preferably, at least about about 5%, 10%, 20%, 30%, 40%, 50%, 60%, 70%, 80%, or about 90% of the entire length of the reference nucleic acid molecule or polypeptide. A fragment may contain about 5, 10, 20, 30, 40, 50, 60, 70, 80, 90, or 100, 200, 300, 400, 500, 600, 700, 800, 900, 1000 or more nucleotides or amino acids.

“Optional” or “optionally” means that the subsequently described event, circumstance, or material may or may not occur or be present, and that the description includes instances where the event, circumstance, or material occurs or is present and instances where it does not occur or is not present.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in some embodiments, to A without B (optionally including elements other than B); in another embodiment, to B without A (optionally including elements other than A); in yet another embodiment, to both A and B (optionally including other elements); etc.

As used herein in the specification and in the claims, “or” should he understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

As used herein an “antigen” is meant to refer to any substance that elicits an immune response.

As used herein, the term “electroporation,” “electro-permeabilization,” or “electro-kinetic enhancement” (“EP”), are used interchangeably and are meant to refer to the use of a transmembrane electric field pulse to induce microscopic pathways (pores) in a bio-membrane; their presence allows biomolecules such as plasmids, oligonucleotides, siRNA, drugs, ions, and/or water to pass from one side of the cellular membrane to the other. In some of the disclosed methods of treatment or prevention, the method comprises a step of electroporation of a subject's tissue for a sufficient time and with a sufficient electrical field capable of inducing uptake of the pharmaceutical compositions disclosed herein into the antigen-presenting cells. In some embodiments, the cells are antigen presenting cells.

The term “pharmaceutically acceptable excipient, carrier or diluent” as used herein is meant to refer to an excipient, carrier or diluent that can be administered to a subject, together with an agent, and which does not destroy the pharmacological activity thereof and is nontoxic when administered in doses sufficient to deliver a therapeutic amount of the agent. The term “pharmaceutically acceptable salt” of nucleic acids as used herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, suifanilic, formic, toluenesulfonie, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenyiacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled viral specific antigens or polynucleotides provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “prevent,” “preventing,” “prevention,” “prophylactic treatment,” and the like, are meant to refer to reducing the probability of developing a disease or condition in a subject, who does not have, but is at risk of or susceptible to developing a disease or condition.

As used herein, the term “purified” means that the polynucleotide or polypeptide or fragment, variant, or derivative thereof is substantially free of other biological material with which it is naturally associated, or free from other biological materials derived, e.g., from a recombinant host cell that has been genetically engineered to express the polypeptide of the invention. That is, e.g., a purified polypeptide of the present disclosure is a polypeptide that is at least from about 70% to about 100% pure, i.e., the polypeptide is present in a composition wherein the polypeptide constitutes from about 70% to about 100% by weight of the total composition. In some embodiments, the purified polypeptide of the present disclosure is from about 75% to about 99% by weight pure, from about 80% to about 99% by weight pure, from about 90 to about 99% by weight pure, or from about 95% to about 99% by weight pure.

The terms ““subject,” “individual,” and “patient” are used interchangeably herein to refer to a vertebrate, preferably a mammal, more preferably a human. Mammals include, but are not limited to, murines, simians, humans, farm animals, cows, pigs, goats, sheep, horses, dogs, sport animals, and pets. Tissues, cells and their progeny obtained in vivo or cultured in vitro are also encompassed by the definition of the term “subject.” The term “subject” is also used throughout the specification in some embodiments to describe an animal from which a cell sample is taken or an animal to which a disclosed cell or nucleic acid sequences have been administered. In some embodiment, the subject is a human. For treatment of those conditions which are specific for a specific subject, such as a human being, the term “patient” may be interchangeably used. In some instances in the description of the present disclosure, the term “patient” will refer to human patients suffering from a particular disease or disorder. In some embodiments, the subject may be a non-human animal. The term “mammal” encompasses both humans and non-humans and includes but is not limited to humans, non-human primates, canines, felines, murines, bovines, equines, caprines, and porcines.

The term “therapeutic effect” as used herein is meant to refer to some extent of relief of one or more of the symptoms of a disorder (e.g., HIV infection) or its associated pathology. A “therapeutically effective amount” as used herein is meant to refer to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. A “therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the invention employed in a pharmaceutical composition at levels lower than that required in order to achieve the desired therapeutic effect and gradually increase the dosage until the desired effect is achieved.

The terms “treat,” “treated,” “treating,” “treatment” and the like as used herein are meant to refer to reducing or ameliorating a disorder and/or symptoms associated therewith (e.g., a HIV or AIDS). “Treating” may refer to administration of the DNA vaccines described herein to a subject after the onset, or suspected onset, of a viral infection. “Treating” includes the concepts of “alleviating,” which refers to lessening the frequency of occurrence or recurrence, or the severity, of any symptoms or other ill effects related to a HIV and/or the side effects associated with viral infection. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered agent adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below. Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained. Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. A nucleic acid will generally contain phosphodiester bonds, although nucleic acid analogs maybe included that may have at least one different linkage, e.g., phosphoramidate, phosphorothioate, phosphorodithioate, or 0-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties.

Nucleic acids containing one or more non-naturally occurring or modified nucleotides are also included within one definition of nucleic acids. The modified nucleotide analog may he located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; 0- and N-alkylated nucleotides, e.g. N6-methyl adenosine are suitable. The 2′-OH-group may be replaced by a group selected from H, OR, R, halo, SH, SR, NH₂, NHR, N₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference in their entireties. Modified nucleotides and nucleic acids may also include locked nucleic acids (LNA), as described in U.S. Patent No. 20020115080, which is incorporated herein by reference. Additional modified nucleotides and nucleic acids are described in U.S. Patent Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made. In some embodiments, the expressible nucleic acid sequence is in the form of DNA. In some embodiments, the expressible nucleic acid is in the form of RNA with a sequence that encodes the polypeptide sequences disclosed herein and, in some embodiments, the expressible nucleic acid sequence is an RNA/DNA hybrid molecule that encodes any one or plurality of polypeptide sequences disclosed herein.

As used herein, the term “nucleic acid molecule” is a molecule that comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also includes a plasmid containing one or more nucleotide sequences that encode one or a plurality of viral antigens. In some embodiments, the disclosure relates to a pharmaceutical composition comprising a first, second, third or more nucleic acid molecule, each of which encoding one or a plurality of viral antigens and at least one of each plasmid comprising one or more of the compositions disclosed herein.

The terms “polypeptide,” “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity,” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may he performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

By “substantially identical” is meant nucleic acid molecule (or polypeptide) exhibiting at least 50% identity to a reference amino acid sequence (for example, any one of the amino acid sequences described herein) or nucleic acid sequence (for example, any one of the nucleic acid sequences described herein). In some embodiments, such a sequence is at least about 60%, 70%, 80% or 85%, 90%, 95% or even 99% identical at the amino acid level or nucleic acid to the sequence used for comparison.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

A “vector” is a nucleic acid that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide. The disclosure relates to any one or plurality of vectors that comprise nucleic acid sequences encoding any one or plurality of amino acid sequence disclosed herein.

The term “vaccine” as used herein is meant to refer to a composition for generating immunity for the prophylaxis and/or treatment of diseases (e.g., viral infections). Accordingly, vaccines are medicaments which comprise antigens in protein and/or nucleic acid forms and are intended to be used in humans or animals for generating specific defense and protective substance by vaccination. A “vaccine composition” or a “DNA vaccine composition” can include a pharmaceutically acceptable excipient, earner or diluent.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. The term “about” as used herein when referring to a measurable value such as an amount, a temporal duration, and the like, is meant to encompass variations of ±20%, +10%, ±5%, ±1%, ±0.5%, or ±0.1% from the specified value, as such variations are appropriate to perform the disclosed methods.

“Variants” is intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” nucleic acid molecule or polypeptide comprises a naturally occurring nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Variants of a particular nucleic acid molecule of the disclosure (i.e., the reference DNA sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ, in some embodiments, from that protein by as few as about 1 to about 15 amino acid residues, as few as about 1 to about 10, such as about 6-to about 10, as few as about 5, as few as 4, 3, 2, or even 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly. In some embodiments, the nucleic acid molecules or the nucleic acid sequences comprise conservative mutations of 1, 2, 3, 4, 5, 6, 7, 8, 9, 10 or more nucleotides.

Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference in their entireties. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### A. Nucleic Acid Compositions

Disclosed are compositions comprising an expressible nucleic acid sequence comprising a first nucleic acid sequence encoding a retroviral trimer polypeptide, a functional fragment thereof or a pharmaceutically acceptable salt thereof. In some embodiments, the disclosure relates to compositions comprising an expressible nucleic acid sequence comprising, in a 5′ to 3′ orientation, a first nucleic acid sequence comprising a leader sequence, functional fragment thereof or a pharmaceutically acceptable salt thereof; and a second nucleic acid sequence encoding a retroviral trimer polypeptide, a functional fragment thereof or a pharmaceutically acceptable salt thereof. In some embodiments, the disclosure relates to compositions comprising an expressible nucleic acid sequence comprising, in a 5′ to 3′ orientation, a first nucleic acid sequence comprising a leader sequence, functional fragment thereof or a pharmaceutically acceptable salt thereof; and a second nucleic acid sequence encoding a retroviral polypeptide that is a component of a retroviral trimer, a functional fragment thereof or a pharmaceutically acceptable salt thereof. In some embodiments, the retroviral polypeptide that is a component of a retroviral trimer is a monomer of a retroviral trimer, such that, upon expression, the monomers spontaneously aggregate to form a trimeric retroviral polypeptide. In some embodiments, the expressible nucleic acid comprises a leader sequence. In some embodiments, the leader is an IgE or IgG leader sequence. In some embodiments, the expressible nucleic acid sequence comprises a first nucleic acid sequence and a second nucleic acid sequence, each of the first and second nucleic acid sequences encoding a retroviral ENV protein or variant thereof, the first and second nucleic acid sequences are non-contiguous and separated by at least one nucleic acid sequence encoding a linker. In some embodiments, the expressible nucleic acid sequence comprises a first nucleic acid sequence and a second nucleic acid sequence, each of the first and second nucleic acid sequences encoding a retroviral ENV protein or variant thereof, the first and second nucleic acid sequences are non-contiguous and separated by at least one nucleic acid sequence encoding a linker, wherein the retroviral ENV protein or variant thereof is free of a transmembrane domain. In some embodiments, the expressible nucleic acid sequence comprises a first nucleic acid sequence and a second nucleic acid sequence, each of the first and second nucleic acid sequences encoding a retroviral ENV protein or variant thereof, the first and second nucleic acid sequences are non-contiguous and separated by at least one nucleic acid sequence encoding a linker, wherein the retroviral ENV protein or variant thereof is free of a transmembrane domain and, upon expression is capable of self-assembly into a trimer. In some embodiments, the expressible nucleic acid sequence comprises a first nucleic acid sequence and a second nucleic acid sequence, each of the first and second nucleic acid sequences encoding a HIV-1 ENV protein or variant thereof, the first and second nucleic acid sequences are non-contiguous and separated by at least one nucleic acid sequence encoding a linker, wherein the HIV-1 ENV protein or variant thereof is free of the native transmembrane domain (gp41) and, upon expression is capable of self-assembly into a trimer. In some embodiments, the expressible nucleic acid sequence comprises a first nucleic acid sequence, a second nucleic acid sequence and a third nucleic acid sequence, each of the first, second and third nucleic acid sequences encoding a retroviral ENV monomer or variant thereof, the first, second and third nucleic acid sequences are non-contiguous and separated by at least one nucleic acid sequence encoding at least one linker.

Disclosed are compositions comprising an expressible nucleic acid sequence comprising a first nucleic acid sequence comprising at least about 70%, 75%, 80%, 85%, 90%, 95%, 99% or 100% sequence identity to one or a plurality of: SEQ ID NO: 1, SEQ ID NO: 2, SEQ ID NO: 3, SEQ ID NO: 4, SEQ ID NO: 5, SEQ ID NO: 6, SEQ ID NO: 8 and SEQ ID NO: 9 or a pharmaceutically acceptable salt thereof; and a second nucleotide sequence comprising at least about 70%, 75%, 80%, 85%, 90%, 95%, 99% or 100% sequence identity to one or a plurality of: SEQ ID NO: 53, SEQ ID NO: 54, SEQ ID NO: 56, SEQ ID NO: 57, SEQ ID NO: 59 SEQ ID NO: 60, SEQ ID NO: 62 SEQ ID NO: 63, SEQ ID NO: 78, SEQ ID NO: 79, SEQ ID NO: 81, SEQ ID NO: 82, SEQ ID NO: 84, SEQ ID NO: 85, SEQ ID NO: 87, SEQ ID NO: 88, SEQ ID NO: 90, SEQ ID NO: 91 or a pharmaceutically acceptable salt of any of the foregoing. In some embodiments, the expressible nucleic acid sequence comprised in the disclosed composition comprises a first nucleic acid sequence encoding a polypeptide comprising at least 70%, 75%, 80%, 85%, 90%, 95%, 99% or 100% sequence identity to one or a plurality of: SEQ ID NO: 7 and SEQ ID NO: 10 or a pharmaceutically acceptable salt thereof; and a second nucleic acid sequence encoding a polypeptide comprising at least 70%, 75%, 80%, 85%, 90%, 95%, 99% or 100% sequence identity to one or a plurality of: SEQ ID NO: 55, SEQ ID NO: 58, SEQ ID NO: 61, SEQ ID NO: 64, SEQ ID NO:80, SEQ ID NO:83. SEQ ID NO:86, SEQ ID NO: 89, SEQ ID NO: 92 or a pharmaceutically acceptable salt of any of the foregoing.

Also disclosed are compositions comprising an expressible nucleic acid sequence comprising a nucleic acid sequence encoding a transmembrane domain free of an HIV ENV transmembrane domain (e.g., gp41). In some embodiments, the transmembrane domain comprises at least about 70% 75%, 80%, 85%, 90%, 95%, 99% or 100% sequence identity to SEQ ID NO: 230, SEQ ID NO: 231 or a pharmaceutically acceptable salt thereof; and a nucleotide sequence encoding a self-assembling polypeptide optionally fused to the transmembrane domain.

In some embodiments, the expressible nucleic acid sequence further comprises a nucleic acid sequence encoding at least one viral antigen or a pharmaceutically acceptable salt thereof. In some embodiments, the expressible nucleic acid sequence further comprises at least one nucleic acid sequence encoding a linker. Thus, also disclosed are compositions comprising an expressible nucleic acid sequence comprising a first nucleic acid sequence encoding a leader sequence or a pharmaceutically acceptable salt thereof; and a second nucleic acid sequence comprising sequence that encodes a self-assembling polypeptide or a pharmaceutically acceptable salt thereof; a third nucleic acid sequence encoding a linker sequence; and a fourth nucleic acid sequence comprising a sequence that encodes at least one viral antigen. In some embodiments, the expressible nucleic acid is operably linked to one or more regulatory sequences. In some embodiments, the expressible nucleic acid is part of a nucleic acid molecule, such as a vector or plasmid.

The disclosure also relates to any of the nucleic acid sequences disclosed herein as RNA, modified RNA or DNA-RNA hybrid molecules or pharmaceutically acceptable salts thereof. If the nucleic acid sequence of the disclosure is prepared as a mRNA sequence, the mRNA sequence may be modified with a polyA tail and/or a 5′ cap at the 5′ end and/or may be modified or encapsulated by lipid or lipid-like of the nucleic acid sequence. The nucleic acid sequences of the disclosure may have any one or a combination of modifications disclosed herein.

In some embodiments, the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In some embodiments, this guanosine is methylated at the 7-position. The term “conventional 5′-cap” refers to a naturally occurring RNA 5′-cap, preferably to the 7-methylguanosine cap (m 7G). In the context of the present disclosure, the term “5′-cap” includes a 5′-cap analog that resembles the RNA cap structure and is modified to possess the ability to stabilize RNA and/or enhance translation of RNA if attached thereto, preferably in vivo and/or in a cell.

The 5′ end of the RNA includes a cap structure having the following general formula:

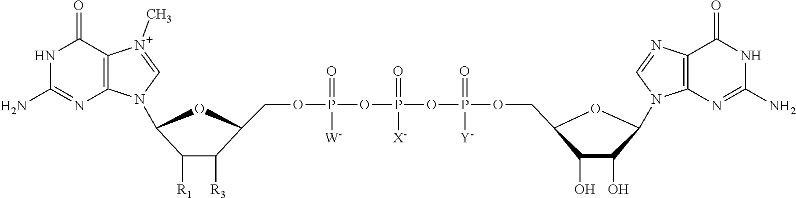

wherein R₁ and R₂ are independently hydroxy or methoxy and W-, X- and Y-are independently oxygen, sulfur, selenium, or BH₃. In some embodiments, R₁ and R₂ are hydroxy and W-, X- and Y- are oxygen. In some embodiments, one of R₁ and R₂, preferably R₁ is hydroxy and the other is methoxy and W-, X- and Y- are oxygen. In some embodiments, R₁ and R₂ are hydroxy and one of W-, X- and Y-, preferably X- is sulfur, selenium, or BH₃, preferably sulfur, while the other are oxygen; and the nucleotide on the right hand side is bonded to the expressible RNA sequence through its 3′ group. In some embodiments, one of R₁ and R₂, preferably R₂ is hydroxy and the other is methoxy and one of W-, X- and Y-, preferably X- is sulfur, selenium, or BH 3, preferably sulfur while the other are oxygen. In some embodiments, the disclosure relates to compositions comprising a nucleotide sequence comprising an expressible RNA sequence encoding any of the one or more proteins disclosed herein.

In some embodiments, the term “modification” relates to modifications made to the expressible nucleic acids in order to tailor the vaccine induced responses. In some embodiments, such modifications comprise creating glycan sites so that glycosylation events can be obtained. In some embodiments, such glycan modifications or mutations decrease the bottom reactivity. In some embodiments, such glycan modifications or mutations increase antigen activity. In some embodiments, the methods of the disclosure are free of activating any mannose-binding lectin or complement process due to such glycan modifications or mutations.

### 1. Leader Sequence

Disclosed are nucleic acid sequences comprising a leader sequence or a pharmaceutically acceptable salt thereof “Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

In some embodiments, the leader sequence can be the nucleic acid sequence of ATGGACTGGACCTGGATTCTGTTCCTGGTGGCCGCCGCCACAAGGGTGCACAGC (SEQ ID NO: 1). In some embodiments, the leader sequence can have at least about 60%, 65%, 70%, 75%, 80%, 85%, 90%, 95%, or 99% sequence identity to SEQ ID NO: 1.

In some embodiments, the leader sequence can be the nucleic acid sequence ATGGACTGGACCTGGAGAATCCTGTTCCTGGTGGCCGCCGCCACCGGCACACAC GCCGATACACACTTCCCCATCTGCATCTTTTGCTGTGGCTGTTGCCATAGGTCCAA GTGTGGGATGTGCTGCAAAACT (SEQ ID NO:). In some embodiments, the leader sequence can have at least about 60%, 65%, 70%, 75%, 80%, 85%, 90%, 95%, or 99% sequence identity to any one or plurality of: SEQ ID NO: 1, SEQ ID NO: 2, SEQ ID NO: 3, SEQ ID NO: 4, SEQ ID NO: 5, SEQ ID NO: 6, SEQ ID NO: 8, and SEQ ID NO: 9. In some embodiments, the leader sequence is encoded as MDWTWRILFLVAAATGTHA (SEQ ID NO: 10) or a functional fragment that has at least about 60%, 65%, 70%, 75%, 80%, 85%, 90%, 95%, or 99% sequence identity to SEQ ID NO: 10. In some embodiments, the expressible nucleic acid sequence comprises a nucleic acid sequence encoding a leader that has at least about 60%, 65%, 70%, 75%, 80%, 85%, 90%, 95%, or 99% sequence identity to MDWTWILFLVAAATRVHS (SEQ ID NO: 7).

### 2. Self-Assembling Polypeptide as Particle Monomer

The disclosure relates to an expressible nucleic acid sequence comprising at least one domain that encodes a self-assembling polypeptide. In some embodiments, the self-assembling polypeptide is encoded by an antigen presenting cell that is transfected or transduced with a nucleic acid molecule comprising the expressible nucleic acid sequence that encodes the self-assembling polypeptide. In some embodiments, self-assembling polypeptides are monomeric forms of retroviral trimers or variants thereof. In some embodiments, the polypeptides are monomers of nanoparticle structural proteins that self-assemble into nanoparticles upon expression. In some embodiments, the nucleotide sequence encoding a self-assembling polypeptide and comprising at least about 70%, 75%, 80%, 85%, 90%, 95%, 96%, 97%, 98%, 99% or 100% sequence identity to SEQ ID NO: 133, SEQ ID NO: 134, SEQ ID NO: 136, SEQ ID NO: 137, SEQ ID NO: 139, SEQ ID NO: 140, SEQ ID NO: 142, SEQ ID NO: 143, SEQ ID NO: 145, SEQ ID NO: 146, SEQ ID NO: 148, SEQ ID NO: 149, SEQ ID NO: 151, SEQ ID NO: 152, or a pharmaceutically acceptable salt thereof. SEQ ID NO: 238 is the DNA sequence encoding lumizine synthase sequence of:

ATGCAGATCTACGAAGGAAAACTGACCGCTGAGGG
ACTGAGGTTCGGAATTGTCGCAAGCCGCGCGAATC
ACGCACTGGTGGATAGGCTGGTGGAAGGCGCTATC
GACGCAATTGTCCGGCACGGCGGGAGAGAGGAAGA
CATCACACTGGTGAGAGTCTGCGGCAGCTGGGAGA
TTCCCGTGGCAGCTGGAGAACTGGCTCGAAAGGAG
GACATCGATGCCGTGATCGCTATTGGGGTCCTGTG
CCGAGGAGCAACTCCCAGCTTCGACTACATCGCCT
CAGAAGTGAGCAAGGGGCTGGCTGATCTGTCCCTG
GAGCTGAGGAAACCTATCACTTTTGGCGTGATTAC
TGCCGACACCCTGGAACAGGCAATCGAGGCGGCCG
GCACCTGCCATGGAAACAAAGGCTGGGAAGCAGCC
CTGTGCGCTATTGAGATGGCAAATCTGTTCAAATC
TCTGCGAGGAGGCTCCGGAGGATCTGGAGGGAGTG
GAGGCTCAGGAGGAGGC.

In some embodiments, the lumizine synthase sequence is derived from hyperthermophilic bacterium Aquifex aeolicus. In some embodiments, other lumizine synthase sequences can be used. In some embodiments, the nucleotide sequence encoding a functional fragment of a self-assembling polypeptide comprising about 60%, 65%, 70%, 75%, 80%, 85%, 90%, 95%, or 99% sequence identity to SEQ ID NO:238. The disclosure also relates to the expressible nucleic acid sequence comprising one or a plurality of self-assembling polypeptides encoded by a first nucleic acid sequence comprising at least 70%, 75%, 80%, 85%, 90%, 95%, or 99% sequence identity to the following:

(3BVE):
GGGCTGAGTAAGGACATTATCAAGCTGCTGAACGA
ACAGGTGAACAAAGAGATGCAGTCTAGCAACCTGT
ACATGTCCATGAGCTCCTGGTGCTATACCCACTCT
CTGGACGGAGCAGGCCTGTTCCTGTTTGATCACGC
CGCCGAGGAGTACGAGCACGCCAAGAAGCTGATCA
TCTTCCTGAATGAGAACAATGTGCCCGTGCAGCTG
ACCTCTATCAGCGCCCCTGAGCACAAGTTCGAGGG
CCTGACACAGATCTTTCAGAAGGCCTACGAGCACG
AGCAGCACATCTCCGAGTCTATCAACAATATCGTG
GACCACGCCATCAAGTCCAAGGATCACGCCACATT
CAACTTTCTGCAGTGGTACGTGGCCGAGCAGCACG
AGGAGGAGGTGCTGTTTAAGGACATCCTGGATAAG
ATCGAGCTGATCGGCAATGAGAACCACGGGCTGTA
CCTGGCAGATCAGTATGTCAAGGGCATCGCTAAGT
CAAGGAAAAGC.

The disclosure also relates to the expressible nucleic acid sequence comprising one or a plurality of self-assembling polypeptides encoded by a first nucleic acid sequence comprising at least 70%, 75%, 80%, 85%, 90%, 95%, 99% or 100% sequence identity to the following:

(RBE):
CTGAGCATTGCCCCCACACTGATTAACCGGGACAA
ACCCTACACCAAAGAGGAACTGATGGAGATTCTGA
GACTGGCTATTATCGCTGAGCTGGACGCCATCAAC
CTGTACGAGCAGATGGCCCGGTATTCTGAGGACGA
GAATGTGCGCAAGATCCTGCTGGATGTGGCCAGGG
AGGAGAAGGCACACGTGGGAGAGTTCATGGCCCTG
CTGCTGAACCTGGACCCCGAGCAGGTGACCGAGCT
GAAGGGCGGCTTTGAGGAGGTGAAGGAGCTGACAG
GCATCGAGGCCCACATCAACGACAATAAGAAGGAG
GAGAGCAACGTGGAGTATTTCGAGAAGCTGAGATC
CGCCCTGCTGGATGGCGTGAATAAGGGCAGGAGCC
TGCTGAAGCACCTGCCTGTGACCAGGATCGAGGGC
CAGAGCTTCAGAGTGGACATCATCAAGTTTGAGGA
TGGCGTGCGCGTGGTGAAGCAGGAGTACAAGCCCA
TCCCTCTGCTGAAGAAGAAGTTCTACGTGGGCATC
AGGGAGCTGAACGACGGCACCTACGATGTGAGCAT
CGCCACAAAGGCCGGCGAGCTGCTGGTGAAGGACG
AGGAGTCCCTGGTCATCCGCGAGATCCTGTCTACA
GAGGGCATCAAGAAGATGAAGCTGAGCTCCTGGGA
CAATCCAGAGGAGGCCCTGAACGATCTGATGAATG
CCCTGCAGGAGGCATCTAACGCAAGCGCCGGACCA
TTCGGCCTGATCATCAATCCCAAGAGATACGCCAA
GCTGCTGAAGATCTATGAGAAGTCCGGCAAGATGC
TGGTGGAGGTGCTGAAGGAGATCTTCCGGGGCGGC
ATCATCGTGACCCTGAACATCGATGAGAACAAAGT
GATCATCTTTGCCAACACCCCTGCCGTGCTGGACG
TGGTGGTGGGACAGGATGTGACACTGCAGGAGCTG
GGACCAGAGGGCGACGATGTGGCCTTTCTGGTGTC
CGAGGCCATCGGCATCAGGATCAAGAATCCAGAGG
CAATCGTGGTGCTGGAG.

The disclosure also relates to the expressible nucleic acid sequence comprising one or a plurality of self-assembling polypeptides encoded by a first nucleic acid sequence comprising at least about 70%, 75%, 80%, 85%, 90%, 95%, 96%, 97%, 98% 99% or 100% sequence identity to the following SEQ ID NO:

### (I3):

GAGAAAGCAGCCAAAGCAGAGGAAGCAGCACGGAA
GATGGAAGAACTGTTCAAGAAGCACAAGATCGTGG
CCGTGCTGAGGGCCAACTCCGTGGAGGAGGCCAAG
AAGAAGGCCCTGGCCGTGTTCCTGGGCGGCGTGCA
CCTGATCGAGATCACCTTTACAGTGCCCGACGCCG
ATACCGTGATCAAGGAGCTGTCTTTCCTGAAGGAG
ATGGGAGCAATCATCGGAGCAGGAACCGTGACAAG
CGTGGAGCAGTGCAGAAAGGCCGTGGAGAGCGGCG
CCGAGTTTATCGTGTCCCCTCACCTGGACGAGGAG
ATCTCTCAGTTCTGTAAGGAGAAGGGCGTGTTTTA
CATGCCAGGCGTGATGACCCCCACAGAGCTGGTGA
AGGCCATGAAGCTGGGCCACACAATCCTGAAGCTG
TTCCCTGGCGAGGTGGTGGGCCCACAGTTTGTGAA
GGCCATGAAGGGCCCCTTCCCTAATGTGAAGTTTG
TGCCCACCGGCGGCGTGAACCTGGATAACGTGTGC
GAGTGGTTCAAGGCAGGCGTGCTGGCAGTGGGCGT
GGGCAGCGCCCTGGTGAAGGGCACACCCGTGGAAG
TCGCTGAGAAGGCAAAGGCATTCGTGGAAAAGATT
AGGGGGTGTACTGAG.

In some embodiments, the expressible nucleic acid sequence comprises of any one or plurality of nucleic acid sequences encoding a self-assembling polypeptide and one or a plurality of nucleic acid sequences encoding a retroviral monomer or trimer. In some embodiments, the compositions or pharmaceutical compositions of the disclosure relate to nucleic acid sequences comprising at least a first expressible nucleic acid sequence comprising a domain with at least about 70%, 75%, 80%, 85%, 90%, 95%, 96%, 97%, 98% 99% or 100% sequence identity to one or a plurality of: SEQ ID NO: 154, SEQ ID NO: 155, SEQ ID NO: 157, SEQ ID NO: 158, SEQ ID NO: 160, SEQ ID NO: 161, SEQ ID NO: 163, SEQ ID NO: 164, SEQ ID NO: 166, SEQ ID NO: 167, SEQ ID NO: 169, SEQ ID NO: 170, SEQ ID NO: 172, SEQ ID NO: 173, SEQ ID NO: 175, SEQ ID NO: 176, SEQ ID NO: 175, SEQ ID NO: 176, SEQ ID NO: 178, and SEQ ID NO: 179.

### 3. Linker

The disclosure relates, in some embodiments, to an expressible nucleic acid sequence comprising a linker that fuses a first domain in a nucleic acid sequence to a second domain in the expressible nucleic acid sequence. In some embodiments, the expressible nucleic acid sequence comprises at least one nucleic acid sequence encoding a linker comprising at least about 70%, 75%, 80%, 85%, 90%, 95%, 96%, 97%, 98%, 99% or 100% sequence identity to SEQ ID NO: 10 or a pharmaceutically acceptable salt thereof. In some embodiments, the expressible nucleic acid sequence has one, two, three, four, five or more linkers in between each antigen domain and each independently selectable from one or a combination of an amino acid sequences at least about 70%, 75%, 80%, 85%, 86%, 87%, 88%, 89%, 90%, 91%, 92%, 93%, 94%, 95%, 96%, 97%, 98%, 99% or 100% sequence identity to: SEQ ID NO: 10, SEQ ID NO: 12, SEQ ID NO: 13, SEQ ID NO: 14, SEQ ID NO: 17, SEQ ID NO: 20, SEQ ID NO: 23, SEQ ID NO: 26, SEQ ID NO: 29, SEQ ID NO: 32, SEQ ID NO: 35, SEQ ID NO: 38, SEQ ID NO: 41, SEQ ID NO: 44, SEQ ID NO: 47, SEQ ID NO: 50 and SEQ ID NO: 52, or a pharmaceutically acceptable salt thereof. In some embodiments, the expressible nucleic acid sequence comprises GACACCATCACACTGCCATGCCGCCCT. In some embodiments, the at least one expressible nucleic acid sequence, encoding a linker, comprises a domain having at least 70%, 75%, 80%, 85%, 90%, 95%, 99% or 100% sequence identity to one or a combination of: SEQ ID NO: 1, SEQ ID NO: 2, SEQ ID NO:3, SEQ ID NO:4, SEQ ID NO: 5, SEQ ID NO: 6, SEQ ID NO: 8, SEQ ID NO: 9, SEQ ID NO: 11, SEQ ID NO: 15, SEQ ID NO: 16, SEQ ID NO: 18, SEQ ID NO: 19, SEQ ID NO: 21, SEQ ID NO: 22, SEQ ID NO: 24, SEQ ID NO: 25, SEQ ID NO: 27, SEQ ID NO:28, SEQ ID NO:30, SEQ ID NO: 31, SEQ ID NO:33 and SEQ ID NO:34 or a pharmaceutically acceptable salt thereof.

The disclosure also relates to the expressible nucleic acid sequence comprising one or a plurality of linker polypeptides encoded by a first nucleic acid sequence comprising at least 70%, 75%, 80%, 85%, 90%, 95%, 99% or 100% sequence identity to the following: GGCGGCTCTGGCGGAAGTGGCGGAAGTGGGGGAAGTGGAGGCGGCGGAAGCGG GGGAGGCAGCGGGGGAGGG. The disclosure also relates to the expressible nucleic acid sequence comprising one or a plurality of linker polypeptides encoded by a first nucleic acid sequence comprising at least 70%, 75%, 80%, 85%, 90%, 95%, 99% or 100% sequence identity to the following: GGCGGAAGCG GCGGAAGCGGCGGGTCT.

In some aspects, the linker polypeptide is GSHSGSGGSGSGGHA or SHSGSGGSGSGGHA, or a polypeptide having 60%, 65%, 70%, 75%, 80%, 85%, 90%, 95%, or 99% sequence identity to SEQ ID NO: 47 or SEQ ID NO: 240.

A linker can be either flexible or rigid or a combination thereof. An example of a flexible linker is a GGS repeat. In some embodiments, the GGS can be repeated about 1, 2, 3, 4, 5, 6, 7, 8, 9, or 10 times, such that the composition comprising a nucleic acid comprises an expressible nucleic acid sequence encoding GGS from an amino terminus to a carboxy terminus in contiguous sequence 1, 2, 3, 4, 5, 6 or more times. An example of a rigid linker is 4QTL-115 Angstroms, single chain 3-helix bundle represented by the sequence:

NEDDMKKLYKQMVQELEKARDRMEKLYKEMVELIQ
KAIELMRKIFQEVKQEVEKAIEEMKKLYDEAKKKI
EQMIQQIKQGGDKQKMEELLKRAKEEMKKVKDKME
KLLEKLKQIMQEAKQKMEKLLKQLKEEMKKMKEKM
EKLLKEMKQRMEEVKKKMDGDDELLEKIKKNIDDL
KKIAEDLIKKAEENIKEAKKIAEQLVKRAKQLIEK
AKQVAEELIKKILQLIEKAKEIAEKVLKGLE.

In some embodiments, the composition comprises a nuclei acid sequence comprising a first expressible nucleic acid sequence comprising 1, 2, 3, 4, 5, 6, 7, 8, 9, 10 or more linkers, each linker is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, each linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, each linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, each linker is about 21 natural or non-natural nucleic acids in length.

In some embodiments, the nucleic acid sequence comprises or consists of Formula I for the expressible nucleic acid (NA) sequence in a 5′ to 3′ orientation:

[NA sequence for Leader Sequence-NA sequence for Viral Antigen Sequence or Self-Assembling Peptide-NA Sequence Linker-NA sequence for Viral Antigen Sequence or Self-Assembling Peptide]. In some embodiments, the multiple cloning site of a plasmid comprises, consists of or consists essentially of Formula I.

In some embodiments, the expressible nucleic acid sequence is within a multiple cloning site of a DNA molecule, such as a plasmid. In some embodiments, the length of each linker according to Formula I is different. For example, in some embodiments, the length of a first linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length, and the length of a second linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length, where the length of the first linker is different from the length of the second linker. Various configurations can be envisioned by the present disclosure, where Formula I comprises 1, 2, 3, 4, 5, 6, 7, 8, 9, 10 or more linkers wherein the linkers are of same, similar or different lengths.

In certain embodiments, two linkers can be used together, in a nucleotide sequence that encodes a fusion peptide. Accordingly, in some embodiments, the first linker is independently selectable from about 0 to about 25 natural or non-natural nucleic acids in length, about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, the second linker is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, the first linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, the second linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length.

### 4. Self-Assembling Polypeptides as Viral Antigens

The disclosure relates to one or a plurality of nucleic acid molecules that comprise at least one expressible nucleic acid sequence, the expressible nucleic acid sequence comprises at least a first nucleic acid sequence encoding a first, a second and/or a third amino acid sequence, each first, second or third amino acid sequence comprising a viral antigen. In some embodiments, the at least first expressible nucleic acid sequence encodes a fusion protein, each fusion protein comprising at least a first, second, and third amino acid sequence contiguously linked by a linker sequence. The disclosure also relates to one or a plurality of nucleic acid molecules that comprise at least one expressible nucleic acid sequence, the expressible nucleic acid sequence comprises at least a first nucleic acid sequence encoding at least one self-assembling polypeptide. In some embodiments, the self-assembling peptide can be at least one self-assembling component of a nanoparticle or at least one retroviral monomer, the retorviral monomer capable of assembling into a retroviral trimer upon expression in a cell. In some embodiments, the at least one expressible nucleic acid sequence comprises nucleic acid sequence encoding a viral antigen free of a nucleic acid sequence encoding a self-assembling nanoparticle polypeptide. In some embodiments, the disclosure relates to a nucleic acid molecule comprising a nucleic acid sequence operably linked to a regulatory sequence and encoding a fusion peptide comprising one or a plurality of self-assembling peptides, wherein at least one of the self-assembling peptides is a self-assembling viral antigen. In some embodiments, upon administration to a subject, the composition comprising a nucleic acid comprising the expressible nucleic acid sequence is transfected or transduced into an antigen presenting cell which encodes the expressible nucleic acid sequence. After a plurality of expressible nucleic acid sequences are encoded, the self-assembling peptide assembles with other self-assembling peptides into a non-native form of a viral antigen. In some embodiments, non-native form of a viral antigen comprises a retroviral trimer exposing an amino acid sequence that is not naturally exposed or free of carbohydrate as compared to the native form or native form of its variant. Expression and presentation of the one or plurality of self-assembling peptides elicits an immune response against an epitope. In some embodiments, the epitope comprises a non-native secondary structure of the one or plurality of self-assembling peptides

In some embodiments, the viral antigen is an HIV-1 ENV protein or variant thereof. In some embodiments, the viral antigen is an HIV-1 ENV protein or a variant thereof. In some embodiments, the viral antigen comprises an HIV-1, strain M, subtype A polypeptide or a variant thereof. In some embodiments, the viral antigen comprises an HIV-1, strain M, subtype B polypeptide or a variant thereof. In some embodiments, the viral antigen comprises an HIV-1, strain M, subtype C polypeptide or a variant thereof. In some embodiments, the viral antigen comprises an HIV-1, strain M, subtype D polypeptide or a variant thereof. In some embodiments, the viral antigen comprises an HIV-1, strain M, subtype E polypeptide or a variant thereof. In some embodiments, the viral antigen comprises an HIV-1, strain M, subtype F polypeptide or a variant thereof. In some embodiments, the viral antigen comprises an HIV-1, strain M, subtype G polypeptide or a variant thereof. In some embodiments, the viral antigen comprises an HIV-1, strain M, subtype H polypeptide or a variant thereof. In some embodiments, the viral antigen comprises an HIV-1, strain M, subtype J polypeptide or a variant thereof. In some embodiments, the viral antigen comprises an HIV-1, strain M, subtype K polypeptide or a variant thereof. In some embodiments, the viral antigen comprises a combination of one or a plurality of HIV-1, strain M polypeptides or variants thereof. In some embodiments, the nucleic acid molecule encodes a fusion peptide comprising one or a plurality of retroviral envelope polypeptides or functional fragments thereof. In some embodiments, the expressible nucleic acid sequence comprises a first nucleic acid sequence encoding, in a 5′ to 3′ orientation, at least three monomers of retroviral ENV proteins. In some embodiments, the at least three monomer polypeptides comprise a furin cleavage site. In some embodiments, the furin cleavage site comprises at least about 80%, 85%, 90%, 95%, 96%, 97%, 98%, 99% or 100% sequence identity to RRRRRR. In some embodiments, the nucleic acid sequence encodes a polypeptide free of carbohydrate proximate to at least 30 amino acids from the carboxy end of the polypeptide. In some embodiments, the nucleic acid sequence encodes a polypeptide free of carbohydrate proximate to at least 20 amino acids from the carboxy end of the polypeptide. In some embodiments, the nucleic acid sequence encodes a polypeptide free of carbohydrate proximate to at least 10 amino acids from the carboxy end of the polypeptide. In some embodiments, the nucleic acid sequence encodes a polypeptide free of carbohydrate proximate to at least 50 amino acids from the carboxy end of the polypeptide.

In some embodiments, the expressible nucleic acid sequence comprises a nucleic acid sequence encoding one, two, three or more monomer or trimer peptides comprising any one or more of the following sequences or a sequence that comprises at least about 80%, 85%, 90%, 95%, 96%, 97%, 98%, 99% or 100% sequence identity to the following sequences in Table X:

TABLE X
BG505_SOSIP_MD39
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQCTNVTN
NITDDMRGELKNCSFNMTTELRDKKQKVYSLFYRLDVVQINENQGNRSNNSNKEYR
LINCNTSAITQACPKVSFEPIPIHYCAPAGFAILKCKDKKFNGTGPCPSVSTVQCTHGIK
PVVSTQLLLNGSLAEEEVIIRSENITNNAKNILVQLNTPVQINCTRPNNNTVKSIRIGPG
QAFYYTGDIIGDIRQAHCNVSKATWNETLGKVVKQLRKHFGNNTIIRFAQSSGGDLE
VTTHSFNCGGEFFYCNTSGLFNSTWISNTSVQGSNSTGSNDSITLPCRIKQIINMWQRI
GQAMYAPPIQGVIRCVSNITGLILLTRDGGSTNSTTETFRPGGGDMRDNWRSELYKYK
VVKIEPLGVAPTRCKRRVVGRRRRRAVGIGAVSLGFLGAAGSTMGAASMTLTVQA
RNLLSGIVQQQSNLLRAPEPQQHLLKDTHWGIKQLQARVLANEHYLRDQQLLGIWG
CSGKLICCTNVPWNSSWSNRNLSEIWDNMTWLQWDKEISNYTQQIIYGLLEESQNQQE
KNEQDLLALD
BG505_MD39_GRSF
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQCTNVTN
NITDDMRGELKNCSFNMTTELRDKKQKVYSLFYRLDVVQINENQGNRSNNSNKEYR
LINCNTSAITQACPKVSFEPIPIHYCAPAGFAILKCKDKKFNGTGPCPSVSTVQCTHGIK
PVVSTQLLLNGSLAEEEVIIRSENITNNAKNILVQLNTPVQINCTRPNNNTVKSIRIGPG
QAFYYTGDIIGDIRQAHCNVSKATWNETLGKVVKQLRKHFGNNTIIRFAQSSGGDLE
VTTHSFNCGGEFFYCNTSGLFNSTWISNTSVQGSNSTGSNDSITLPCRIKQIINMWQRI
GQAMYAPPIQGVIRCVSNITGLILTRDGGSTNSTTETFRPGGGDMRDNWRSELYKYK
VVKIEPLGVAPTRCKRRVVGRRRRRRAVGIGAVSLGFLGAAGSTMGAASMTLTVQA
RNLLSGIVQQQSNLLRAPEPQQHLLKDTHWGIKQLQARVLAVEHYLRDQQLLGIWG
CSGKLICCTNVPWNSSWSNRNLSEIWDNMTWLNWSKEISNYTQIIYGLLEESQNQQE
KNNQSLLALD
BG505_SOSIP_MD39_CPG9.2
GGNSSGSLGFLGAAGSTMGAASMTLTVQARNLLSGIVQQQSNLLRAPEPQQH
LLKDTHWGIKQLQARVLAVEHYLRDQQLLGIWGCSGKLICCTNVPWNSSWSNRNLS
EIWDNMTWLNWSKEISNYTQIIYGLLEESQNQNESNEQDLGGNGSGGGSGSGGNGSS
GLWVTVYYGVPVWKDAETTLFCASDAKAYETEKHNVWATHACVPTDPNPQEIHLE
NVTEEFNMWEKNNMVEQMHEDIISLWDQSLKPCVKLTPLCVTLQCTNVTNNITDDM
RGELKNCSFNMTTELRDKKQKVYSLFYRLDVVQINENQGNRSNNSNKEYRLINCNT
SAITQACPKVSFEPIPTIHYCAPAGFAILKCKDKKFNGTGPCPSVSTVQCTHGIKPVVST
QLLLNGSLAEEEVIIRSENITNNAKNILVQLNTPVQINCTRPNNNTVKSIRIGPGQAFY
YTGDIIGDIRQAHCNVSKATWNETLGKVVKQLRKHFGNNTIRFAQSSGGDLEVTTH
SFNCGGEFFYCNTSGLFNSTWISNTSVQGSNSTGSNDSITLPCRIKQIINMWQRIGQAM
YAPPIQGVIRCVSNITGLILTRDGGSTNSTTETFRPGGGDMRDNWRSELYKYKVVKIE
PLGVAPTRCNRS
BG505_SOSIP_MD39_link14
AENLWVTVYYGVPVWKDAETTLFCASDAKAYETEKHNVWATHACVPTDPN
PQEIHLENVTEEFNMWKNNMVEQMHEDIISLQDQSLKPCVKLTPLCVTLQCTNVTN
NITDDMRGELKNCSFNMTTELRDKKQKVYSLFYRLDVVQINENQGNRSNNSNKEYR
LINCNTSAITQACPKVSFEPIPIHYCAPAGFAILKCKDKKFNGTGPCPSVSTVQCTHGIK
PVVSTQLLLNGSLAEEEVIIRSENITNNAKNILVQLNTPVQINCTRPNNNTVKSIRIGPG
QAFYYTGDIIGDIRQAHCNVSKATWNETLGKVVKQLRKHFGNNTIIRFQSSGGDLE
VTTHSFNCGGEFFYCNTSGLFNSTWISNTSVQGSNSTGSNDSITLPCRIKQIINMWQRI
GQAMYAPPIQGVIRCVSNITGLILTRDGGSTNSTTETFRPGGGDMRDNWRSELYKYK
VVKIEPLGVAPTRCKRRVVGSHSGSGGSGSGGHAAVGIGAVSLGFLGAAGSTMGAA
SMTLTVQARNLLSGIVQQQSNLLRAPEPQQHLLKDTHWGIKQLQARVLAVEHYLRD
QQLLGIWGCSGKLICCTNVPWNSSWSNRNLSEIWDNMTWLQWDKEISNYTQIIYGLL
EESQNQQEKNEQDLLALD
BG505_SOSIP_MD39_trimer string 1 monomer 1
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQCTNVTN
NITDDMRGELKNCSFNMTTELRDKKQKVYSLFYRLDVVQINENQGNRSNNSNKEYR
LINCNTSAITQACPKVSFEPIPIHYCAPAGFAILKCKDKKFNGTGPCPSVSTVQCTHGIK
PVVSTQLLLNGSLAEEEVIIRSENITNNAKNILVQLNTPVQINCTRPNNNTVKSIRIGPG
QAFYYTGDIIGDIRQAHCNVSKATWNETLGKVVKQLRKHFGNNTIIRFAQSSGGDLE
VTTHSFNCGGEFFYCNTSGLFNSTWISNTSVQGSNSTGSNDSITLPCRIKQIINMWQRI
GQAMYAPPIQGVIRCVSNITGLILTRDGGSTNSTTETFRPGGGDMRDNWRSELYKYK
VVKIEPLGVAPTRCKRRVVGSHSGSGGSGSGGHAAVGIGAVSLGFLGAAGSTMGAA
SMTLTVQARNLLSGIVQQQSNLLRAPEPQQHLLKDTHWGIKQLQARVLAVEHYLRD
QQLLGIWGCSGKLICCTNVPWNSSWSNRNLSEIWDNMTWLQWDKEISNYTQIIYGLL
EESQNQQEKNEQDLLALD
BG505_SOSIP_MD39_trimer string 1 monomer 2
AENLLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQCTNVTN
NITDDMRGELKNCSFNMTTELRDKKQKVYSLFYRLDVVQINENQGNRSNNSNKEYR
LINCNTSAITQACPKVSFEPIPIHYCAPAGFAILKCKDKKFNGTGPCPSVSTVQCTHGIK
PVVSTQLLLNGSLAEEEVIIRSENITNNAKNILVQLNTPVQINCTRPNNNTVKSIRIGPG
QAFYYTGDIIGDIRQAHCNVSKATWNETLGKVVKQLRKHFGNNTIIRFAQSSGGDLE
VTTHSFNCGGEFFYCNTSGLFNSTWISNTSVQGSNSTGSNDSITLPCRIKQIINMWQRI
GQAMYAPPIQGVIRCVSNITGLILTRDGGSTNSTTETFRPGGGDMRDNWRSELYKYK
VVKIEPLGVAPTRCKRRVVGSHSGSGGSGSGGHAAVGIGAVSLGFLGAAGSTMGAA
SMTLTVQARNLLSGIVQQQSNLLRAPEPQQHLLKDTHWGIKQLQARVLAVEHYLRD
QQLLGIWGCSGKLICCTNVPWNSSWSNRNLSEIWDNMTWLQWDKEISNYTQIIYGLL
EESQNQQEKNEQDLLALD
BG505_SOSIP_MD39_trimer string 1 monomer 3
AENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDD
MRGELKNCSFNMTTELRDKKQKVYSLFYRLDVVQINENQGNRSNNSNKEYRLINCN
TSAITQACPKVSFEPIPIHYCAPAGFAILKCKDKKFNGTGPCPSVSTVQCTHGIKPVVS
TQLLLNGSLAEEEVIIRSENITNNAKNILVQLNTPVQINCTRPNNNTVKSIRIGPGQAFY
YTGDIIGDIRQAHCNVSKATWNETLGKVVKQLRKHFGNNTIIRFAQSSGGDLEVTTH
SFNCGGEFFYCNTSGLFNSTWISNTSVQGSNSTGSNDSITLPCRIKQIINMWQRIGQAM
YAPPIQGVIRCVSNITGLILTRDGGSTNSTTETFRPGGGDMRDNWRSELYKYKVVKIE
PLGVAPTRCKRRVVGSHSGSGGSGSGGHAAVGIGAVSLFFLGAAGSTMGAASMTLT
VQARNLLSGIVQQQSNLLRAPEPQQHLLKDTHWGIKQLQARVLAVEHYLRDQQLLG
IWGCSGKLICCTNVPWNSSWSNRNLSEIWDNMTWLQWDKEISNYTQIIYGLLEESQN
QQEKNEQDLLALD
BG505_SOSIP_MD39_trimer string 1
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQCTVNTN
NITDDMRGELKNCSFNMTTELRDKKQKVYSLFYRLDVVQINENQGNRSNNSNKEYR
LINCNTSAITQACPKVSFEPIPIHYCAPAGFAILKCKDKKFNGTGPCPSVSTVQCTHGIK
PVVSTQLLLNGSLAEEEVIIRSENITNNAKNILVQLNTPVQINCTRPNNNTVKSIRIGPG
QAFYYTGDIIGDIRQAHCNVSKATWWNETLGKVVKQLRKHFGNNTIIRFAQSSGGDLE
VTTHSFNCGGEFFYCNTSGLFNSTWISNTSVQGSNSTGSNDSITLPCRIKQIINMWQRI
GQAMYAPPIQGVIRCVSNITGLILTRDGGSTNSTTETFRPGGGDMRDNWRSELYKYK
VVKIEPLGVAPTRCKRRVVGSHSGSGGSGSGGHAAVIGIGAVSLGFLGAAGSTMGAA
SMTLTVQARNLLSGIVQQQSNLLRAPEPQQHLLKDTHWGIKQLQARVLAVEHYLRD
QQLLGIWGCSGKLICCTNVPWNSSWSNRNLLSEIWDNMTWLQWDKEISNYTQIIYGLL
EESQNQQEKNEQDLLALDGGAENLWVTVYYGVPVWKDAETTLFCASDAKAYETEK
HNVWATHACVPTDPNPQEIHLENVTEEFNMWKNNMVEQMHEDIISLLWDQSLKPCV
KLTPLCVTLQCTNVTNNITDDMRGELKNCSFNMTTELRDKKQKVYSLFYRLLDVVQI
NENQGNRSNNSNKEYRLINCNTSAITQACPKVSFEPIPIHYCAPAGFAILKCKDKKFN
GTGPCPSVSTVQCTHGIKPVVSTQLLLNGSLAEEEVIIRSENITNNAKNILLVQLNTPVQI
NCTRPNNNTVKSIRIGPGQAFYYTGDIIGDIRQAHCNVSKATWNETLGKVVKQLRKH
FGNNTIIRFAQSSGGDLEVTTHSFNCGGEFFYCNTSGLLFNSTWISNTSVQGSNSTGSND
SITLPCRIKQIINMWQRIGQAMYAPPIQGVIRCVSNITGLILTRDGGSTNSTTETFRPGG
GDMRDNWRSELLYKYKVVKIEPLGVAPTRCKRRVVGSHSGSGGSGSGGHAAVIGIGA
VSLGFLGAAGSTMGAASMTLTVQARNLLSGIVQQQSNLLRAPEPQQHLLKDTHWGI
KQLQARVLAVEHYLRDQQLLGIWGCSGKLICCTNVPWNSSWSNRNLSEIWDNMTW
LQWDKEISNYTQIIYGLLEESQNQQEKNEQDLLALDGGAENLWVTVYYGVPVWKD
AETTLFCASDAKAYETEKHNVWATHACVPTDPNPQEIHLENVTEEFNMWKNNMVE
QMHEDIISLWDQSLKPCVKLTPLCVTLQCTVNTNNITDDMRGELKNCSFNMTTELRD
KKQKVYSLFYRLDVVQINENQGNRSNNSNKEYRLINCNTSAITQACPKVSFEPIPIHY
CAPAGFAILKCKDKKFNGTGPCPSVSTVQCTHGIKPVVSTQLLLNGSLAEEEVIIRSEN
ITNNAKNILLVQLNTPVQINCTRPNNNTVKSIRIGPGQAFYYTGDIIGDIRQAHCNVSKA
TWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLIL
TRDGGSTNSTTETFRPGGGDMRDNWRSELYKYKVVKIEPLGVAPTRCKRRVVGSHS
GSGGSGSGGHAAVGIGAVSLGFLGAAGSTMGAASMTLTVQARNLLSGIVQQQSNLL
RAPEPQQHLLKDTHWGIKQLQARVLAVEHYLRDQQLLGIWWGCSGKLICCTNVPWNS
SWSNRNLSEIWDNMTWLQWDKEISNYTQIIYGLLEESQNQQEKNEQDLLALD
BG505_SOSIP_MD39_trimer string 2 (TS2)
AENLWVTVYYGVPVWKDAETTLFCASDAKAYETEKHNVWATHACVPTDPN
PQEIHLENVTEEFNMWKNNMVEQMHEDIISLWDQSLKPCVKLTPLLCVTLQCTNVTN
NITDDMRGELKNCSFNMTTELRDKKQKVYSLFYRLDVVQINENQGNRSNNSNKEYR
LINCNTSAITQACPKVSFEPIPIHYCAPAGFAILKCKDKKFNGTGPCPSVSTVQCTHGIK
PVVSTQLLLNGSLAEEEVIIRSENITNNAKNILVQLNTPVQINCTRPNNNTVKSIRIGPG
QAFYYTGDIIGDIRQAHCNVSKATWNETLGKVVKQLRKHFGNNTIIRFAQSSGGDLE
VTTHSFNCGGEFFYCNTSGLFNSTWISNTSVQGSNSTGSNDSITLPCRIKQIINMWQRI
GQAMYAPPIQGVIRCVSNITGLILTRDGGSTNSTTETFRPGGGDMRDNWRSELLYKYK
VVKIEPLLGVAPTRCKRRVVGSHSGSGGSGSGGHAAVGIGAVSLGFLGAAGSTMGAA
SMTLTVQARNLLSGIVQQQSNLLRAPEPQQHLLKDTHWGIKQLQARVLAVEHYLRD
QQLLGIWGCSGKLICCTNVPWNSSWSNRNLSEIWDNMTWLQWDKEISNYTQIIYGLL
EESQNQQEKNEQDLLALDGGSGSGAENLWVTVYYGVPVWKDAETTLFCASDAKAY
ETEKHNVWATHACVPTDPNPQEIHLENVTEEFNMWKNNMVEQMHEDIISLWDQSL
KPCVKLTPLCVTLQCTNVTNNITDDMRGELKNCSFNMTTELRDKKQKVYSLFYRLD
VVQINENQGNRSNNSNKEYRLINCNTSAITQACPKVSFEPIPIHYCAPAGFAILKCKDK
KFNGTGPCPSVSTVQCTHGIKPVVSTQLLLNGSLAEEEVIIRSENITNNAKNILVQLNT
PVQINCTRPNNNTVKSIRIGPGQAFYYTGDIIGDIRQAHCNVSKATWNETLGKVVKQL
RKHFGNNTIIRFAQSSGGDLEVTTHSFNCGGEFFYCNTSGLFNSTWISNTSVQGSNST
GSNDSITLPCRIKQIINMWQRIGQAMYAPPIQGVIRCVSNITGLILTRDGGSTNSTTETF
RPGGGDMRDNWRSELYKYKVVKIEPLGVAPTRCKRRVVGSHSGSGGSGSGGHAAV
GIGAVSLGFLGAAGSTMGAASMTLTVQARNLLSGIVQQQSNLLRAPEPQQHLLKDT
HWGIKQLQARVLAVEHYLRDQQLLGIWGCSGKLICCTNVPWNSSWSNRNLSEIWDN
MTWLQWDKEISNYTQIIYGLLEESQNQQEKNEQDLLALDGGSGSGAENLWVTVYY
GVPVWKDAETTLFCASDAKAYETEKHNVWATHACVPTDPNPQEIHLENVTEEFNM
WKNNMVEQMHEDIISLWDQSLKPCVKLTPLCVTLQCTNVTNNITDDMRGELKNCSF
NMTTELRDKKQKVYSLFYRLDVVQINENQGNRSNNSNKEYRLINCNTSAITQACPKV
SFEPIPIHYCAPAGFAILKCKDKKFNGTGPCPSVSTVQCTHGIKPVVSTQLLLNGSLAE
EEVIIRSENITNNAKNILVQLNTPVQINCTRPNNNTVKSIRIGPGQAFYYTGDIIGDIRQ
AHCNVSKATWNETLGKVVKQLRKHFGNNTIIRFAQSSGGDLEVTTHSFNCGGEFFYC
NTSGLFNSTWISNTSVQGSNSTGSNDSITLPCRIKQIINMWQRIGQAMYAPPIQGVIRC
VSNITGLILTRDGGSTNSTTETFRPGGGDMRDNWRSELYKYKVVKIEPLGVAPTRCK
RRVVGSHSGSGGSGSGGHAAVGIGAVSLGFLGAAGSTMGAASMTLTVQARNLLSGI
VQQQSNLLRAPEPQQHLLKDTHWGIKQLQARVLAVEHYLRDQQLLGIWGCSGKLIC
CTNVPWNSSWSNRNLSEIWDNMTWLQWDKEISNYTQIIYGLLEESQNQQEKNEQDL
LALD
BG505_MD39
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQCTNVTN
NITDDMRGELKNCSFNMTTELRDKKQKVYSLFYRLDVVQINENQGNRSNNSNKEYR
LINCNTSAITQACPKVSFEPIPHYCAPAGFAILKCKDKKFNGTGPCPSVSTVQCTHGIK
PVVSTQLLLNGSLAEEEVIIRSENITNNAKNILVQLNTPVQINCTRPNNNTVKSIRIGPG
QAFYYTGDIIGDIRQAHCNVSKATWNETLGKVVKQLRKHFGNNTIIRFAQSSGGDLE
VTTHSFNCGGEFFYCNTSGLFNSTWISNTSVQGSNSTGSNDSITLPCRIKQIINMWQRI
GQAMYAPPIQGVIRCVSNITGLILTRDGGSTNSTTETFRPGGGDMRDNWRSELYKYK
VVKIEPLGVAPTRCKRRVVGSHSGSGGSGSGGHAAVGIGAVSLGFLGAAGSTMGAA
SMTLTVQARNLLSGIVQQQSNLLRAPEPQQHLLKDTHWGIQLQARVLAVEHYLRD
QQLLGIWGCSGKLICCTNVPWNSSWSNRNLSEIWDNMTWLQWDKEISNYTQIIYGLL
EESQNQQEKNEQDLLALD
BG505_MD39+linker
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQCTNVTN
NITDDMRGELKNCSFNMTTELRDKKQKVYSLFYRLDVVQINENQGNRSNNSNKEYR
LINCNTSAITQACPKVSFEPIPIHYCAPAGFAILKCKDKKFNGTGPCPSVSTVQCTHGIK
PVVSTQLLLNGSLAEEEVIIRSENITNNAKNILVQLNTPVQINCTRPNNNTVKSIRIGPG
QAFYYTGDIIGDIRQAHCNVSKATWNETLGKVVKQLRKHFGNNTIIRFAQSSGGDLE
VTTHSFNCGGEFFYCNTSGLFNSTWISNTSVQGSNSTGSNDSITLPCRIKQIINMWQRI
GQAMYAPPIQGVIRCVSNITGLILTRDGGSTNSTTETFRPGGGDMRDNWRSELYKYK
VVKIEPLGVAPTRCKRRVVGSHSGSGGSGSGGHAAVGIGAVSLGFLGAAGSTMGAA
SMTLTVQARNLLSGIVQQQSNLLRAPEPQQHLLKDTHWGIKQLQARVLAVEHYLRD
QQLLGIWGCSGKLICCTNVPWNSSWSNRNLSEIWDNMTWLQWDKEISNYTQIIYGLL
EESQNQQEKNEQDLLALDGGGSGGSGGSGGSGGSGGS
BG505_MD39_link14_gp140-PDGFR
AENLWVTVYYGVPVWEKDAETTLFCASDAKAYETEKHNVWATHACVPTDPN
PQEIHLENVTEEFNMWKNNMVEQMHEDIISLWDQSLKPCVKLTPLCVTLQCTNVTN
NITDDMRGELKNCSFNMTTELRDKKQKVYSLFYRLDVVQINENQGNRSNNSNKEYR
LINCNTSAITQACPKVSFEPIPIHYCAPAGFAILKCKDKKFNGTGPCPSVSTVQCTHGIK
PVVSTQLLLNGSLAEEEVIIRSENITNNAKNILVQLNTPVQINCTRPNNNTVKSIRIGPG
QAFYYTGDIIGDIRQAHCNVSKATWNETLGKVVKQLRKHFGNNTIIRFAQSSGGDLE
VTTHSFNCGGEFFYCNTSGLFNSTWISNTSVQGSNSTGSNDSITLPCRIKQIINMWOQRI
GQAMYAPPIQGVIRCVSNITGLILTRDGGSTNSTTETFRPGGGDMRDNWRSELYKYK
VVKIEPLGVAPTRCKRRVVGSHSGSGGSGSGGHAAVGIGAVSLGFLGAAGSTMGAA
SMTLTVQARNLLSGIVQQQSNLLRAPEPQQHLLKDTHWGIKQLQARVLAVEHYLRD
QQLLGIWGCSGKLICCTNVPWNSSWSNRNLSEIWDNMTWLQWDKEISNYTQIIYGLL
EESQNQQEKNEQDLLALDGGGSGGSGGSGGSGGSGGSNAVGQDTQEVIVVPHSLPF
KVVVISAILALVVLTIISLILLIMLWQKKPR
BG505_MD39_gp140_foldon-PDGFR
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQCTNVTN
NITDDMRGELKNCSFNMTTELRDKKQKVYSLFYRLDVVQINENQGNRSNNSNKEYR
LINCNTSAITQACPKVSFEPIPIHYCAPAGFAILKCKDKKFNGTGPCPSVSTVQCTHGIK
PVVSTQLLLNGSLAEEEVIIRSENITNNAKNILVQLNTPVQINCTRPNNNTVKSIRIGPGP
QAFYYTGDIIGDIRQAHCNVSKATWNETLGKVVKQLRKHFGNNTIIRFAQSSGGDLE
VTTHSFNCGGEFFYCNTSGLFNSTWISNTSVQGSNSTGSNDSITLPCRIKQIINMWQRI
GQAMYAPPIQGVIRCVSNITGLILTRDGGSTNSTTETFRPGGGDMRDNWRSELYKYK
VVKIEPLGVAPTRCKRRVVGSHSGSGGSGSGGHAAVGIGAVSLGFLGAAGSTMGAA
SMTLTVQARNLLSGIVQQQSNLLRAPEPQQHLLKDTHWGIKQLQARVLAVEHYLRD
QQLLGIWGCSGKLICCTNVPWNSSWSNRNLSEIWDNMTWLQWDKEISNYTQIIYGLL
EESQNQQEKNEQDLLALDGGGSGGSGGGYIPEAPRDGQAYVRKDGEWVLLSTFLGG
SGGSGGSGGSNAVGQDTQEVIVVPHSLPFKVVVISAILALVVLTIISLIILIMLWQKKPR
BG505_MD39_TS1_gp140
AENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQCTNVTN
NITDDMRGELKNCSFNMTTELRDKKQKVYSLFYRLDVVQINENQGNRSNNSNKEYR
LINCNTSAITQACPKVSFEPIPIHYCAPAGFAILKCKDKKFNGTGPCPSVSTVQCTHGIK
PVVSTQLLLNGSLAEEEVIIRSENITNNAKNILVQLNTPVQINCTRPNNNTVKSIRIGPG
QAFYYTGDIIGDIRQAHCNVSKATWNETLGKVVKQLRKHFGNNTIIRFAQSSGGDLE
VTTHSFNCGGEFFYCNTSGLFNSTWISNTSVQGSNSTGSNDSITLPCRIKQIINMWQRI
GQAMYAPPIQGVIRCVSNITGLILTRDGGSTNSTTETFRPGGGDMRDNWRSELYKYK
VVKIEPLGVAPTRCKRRVVGSHSGSGGSGSGGHAAVGIGAVSLGFLGAAGSTMGAA
SMTLTVQARNLLSGIVQQQSNLLRAPEPQQHLLKDTHWGIKQLQARVLAVEHYLRD
QQLLGIWGCSGKLICCTNVPWNSSWSNRNLSEIWDNMTWLQWDKEISNYTQIIYGLL
EESQNQQEKNEQDLLALDGGAENLWVTVYYGVPVWKDAETTLFCASDAKAYETE
KHNVWATHACVPTDPNPQEIHLENVTEEFNMWKNNMVEQMHEDIISLWDQSLKPC
VKLTPLCVTLQCTNVTNNITDDMRGELKNCSFNMTTELRDKKQKVYSLFYRLDVVQ
INENQGNRSNNSNKEYRLINCNTSAITQACPKVSFEPIPIHYCAPAGFAILKCKDKKFN
GTGPCPSVSTVQCTHGIKPVVSTQLLLNGSLAEEEVIIRSENITNNAKNILVQLNTPVQT
NCTRPNNNTVKSIRIGPGQAFYYTGDIIGDIRQAHCNVSKATWNETLGKVVKQLRKH
FGNNTIRFAQSSGGDLEVTTHSFNCGGEFFYCNTSGLFNSTWISNTSVQGSNSTGSND
SITLPCRIKQIINMWOQRIGQAMYAPPIQGVIRCVSNITGLILTRDGGSTNSTTETFRPGG
GDMRDNWRSELYKYKVVKIEPLGVAPTRCKRRVVGSHSGSGGSGSGGHAAVGIGA
VSLGFLGAAGSTMGAASMTLTVQARNLLSGIVQQQSNLLRAPEPQQHLLKDTHWGI
KQLQARVLAVEHYLRDQQLLGIWGCSGKLICCTNVPWNSSWSNRNLSEIWDNMTW
LQWDKEISNYTQIIYGLLEESQNQQEKNEQDLLALDGGAENLWVTVYYGVPVWEKD
AETTLFCASDAKAYETEKHNVWATHACVPTDPNPQEIHLENVTEEFNMWKNNMVE
QMHEDIISLWDQSLKPCVKLTPLCVTLQCTNVTNNITDDMRGELKNCSFNMTTELRD
KKQKVYSLFYRLDVVQINENQGNRSNNSNKEYRLINCNTSAITQACPKVSFEPIPIHY
CAPAGFAILKCKDKKFNGTGPCPSVSTVQCTHGIKPVVSTQLLLNGSLAEEEVIIRSEN
ITNNAKNILVQLNTPVQINCTRPNNNTVKSIRIGPGQAFYYTGDIIGDIRQAHCNVSKA
TWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLIL
TRDGGSTNSTTETFRPGGGDMRDNWRSELYKYKVVKIEPLGVAPTRCKRRVVGSHS
GSGGSGSGGHAAVGIGAVSLGFLGAAGSTMGAASMTLTVQARNLLSGIVQQQSNLL
RAPEPQQHLLKDTHWGIKQLQARVLAVEHYLRDQQLLGIWGCSGKLICCTNVPWNS
SWSNRNLSEIWDNMTWLQWDKEISNYTQIIYGLLEESQNQQEKNEQDLLALD
BG505_MD39_TS1_gp140-PDGFR
AENLWVTVYYGVPVWEKDAETTLFCASDAKAYETEKHNVWATHACVPTDPN
PQEIHLENVTEEFNMWKNNMVEQMHEDIISLWDQSLKPCVKLTPLCVTLQCTNVTN
NITDDMRGELKNCSFNMTTELRDKKQKVYSLFYRLDVVQINENQGNRSNNSNKEYR
LINCNTSAITQACPKVSFEPIPIHYCAPAGFAILKCKDKKFNGTGPCPSVSTVQCTHGIK
PVVSTQLLLNGSLAEEEVIIRSENITNNAKNILVQLNTPVQINCTRPNNNTVKSIRIGPG
QAFYYTGDIIGDIRQAHCNVSKATWNETLGKVVKQLRKHFGNNTIIRFAQSSGGDLE
VTTHSFNCGGEFFYCNTSGLFNSTWISNTSVQGSNSTGSNDSITLPCRIKQIINMWQRI
GQAMYAPPIQGVIRCVSNITGLILTRDGGSTNSTTETFRPGGGDMRDNWRSELYKYK
VVKIEPLGVAPTRCKRRVVGSHSGSGGSGSGGHAAVGIGAVSLGFLGAAGSTMGAA
SMTLTVQARNLLSGIVQQQSNLLRAPEPQQHLLKDTHWGIKQLQARVLAVEHYLRD
QQLLGIWGCSGKLICCTNVPWNSSWSNRNLSEIWDNMTWLQWDKEISNYTQIIYGLL
EESQNQQEKNEQDLLALDGGAENLWVTVYYGVPVWDKAETTLFCASDAKAYETEK
HNVWATHACVPTDPNPQEIHLENVTEEFNMWKNNMVEQMHEDIISLWDQSLKPCV
KLTPLCVTLQCTNVTNNITDDMRGELKNCSFNMTTELRDKKQKVYSLFYRLDVVQI
NENQGNRSNNSNKEYRLINCNTSAITQACPKVSFEPIPIHYCAPAGFAILKCKDKKFN
GTGPCPSVSTVQCTHGIKPVVSTQLLLNGSLAEEEVIIRSENITNNAKNILVQLNTPVQI
NCTRPNNNTVKSIRIGPGQAFYYTGDIIGDIRQAHCNVSKATWNETLGKVVKQLRKH
FGNNTIIRFAQSSGGDLEVTTHSFNCGGEFFYCNTSGLFNSTWISNTSVQGSNSTGSND
SITLPCRIKQIINMWQRIGQAMYAPPIQGVIRCVSNITGLILTRDGGSTNSTTETFRPGG
GDMRDNWRSELYKYKVVKIEPLGVAPTRCKRRVVGSHSGSGGSGSGGHAAVGIGA
VSLGFLGAAGSTMGAASMTLTVQARNLLSGIVQQQSNLLRAPEPQQHLLKDTHWGI
KQLQARVLAVEHYLRDQQLLGIWGCSGKLICCTNVPWNSSSWSNRNLSEIWDNMTW
LQWDKEISNYTQIIYGLLEESQNQQEKNEQDLLALDGGAENLWVTVYYGVPVWKD
AETTLFCASDAKAYETEKHNVWATHACVPTDPNPQEIHLENVTEEFNMWKNNMVE
QMHEDIISLWDQSLKPCVKLTPLCVTLQCTNVTNNITDDMRGELKNCSFNMTTELRD
KKQKVYSLFYRLDVVQINENQGNRSNNSNKEYRLINCNTSAITQACPKVSFEPIPIHY
CAPAGFAILKCKDKKFNGTGPCPSVSTVQCTHGIKPVVSTQLLLNGSLAEEEVIIRSEN
ITNNAKNILVQLNTPVQINCTRPNNNTVKSIRIGPGQAFYYTGDIIGDIRQAHCNVSKA
TWENTLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLIL
TRDGGSTNSTTETFRPGGGDMRDNWRSELYKYKVVKIEPLGVAPTRCKRRVVGSHS
GSGGSGSGGHAAVGIGAVSLGFLGAAGSTMGAASMTLTVQARNLLSGIVQQQSNLL
RAPEPQQHLLKDTHWGIKQLQARVLAVEHYLRDQQLLGIWGCSGKLICCTNVPWNS
SWSNRNLSEIWDNMTWLQWDKEISNYTQIIYGLLEESQNQQEKNEQDLLALDGGGS
GGSGGSGGSGGSGGSNAVGQDTQEVIVVPHSLPFKVVVISAILALVVLTIISLIILIML
WQKKPR
TRO11_AY835445_MD39_L14G8
MDWTWILFLVAAATRVHSQGQLWVTVYYGVPVWKDASTTLFCASDAYAK
DTEVHNVWATHACVPTDPNPQEVVLGNVTENFNMWKNNMVDQMHEDIISLWDQS
LKPCVKLTPLCVTLNCTDNITNTNTNSSKNSSTHSYNNSLEGEMKNCSFNITAGIRDK
VKKEYALFYKLDVVPIEEDKDTNKTTYRLRSCNTSVITQACPKVTFEPIPIHYCAPAG
FAILCNDKKFNGTGPCTNVSTVQCTHGIRPVVSTQLLLNGSLAEEEVIIRSENFTNNA
KTIIVQLNESIANINCTRPNNNTVRSIHIGPGRAFYYTGDIIGDIRQAHCNISRTEWNSTL
RQIVTKLREQLGDPNKTIIFAQSSGGDTEITMHSFNCGGEFFYCNTTKLFNSTWNGNN
TTESDSTGENITLPCRIKQIINLWQEVGKAMYAPPIKGQISCSSNITGLLLTRDGGNNN
SSGPETFRPGGGNMKDNWRSELYKYKVIKIEPLGVAPTRCKRRVVGSHSGSGGSGSGS
GHAAVGTLGAMSLGFGAAGSTMGAASVTLTVQARLLLSGIVQQQNNLLRAPEQQ
HMLQDTHWGIKQLQARVLAVEHYLRDQQLLGIWGCSGKLICCTNVPWNASWSNKS
LNNIWENMTWMNWSREIDNYTDLIYILLEKSQIQQEKNNQSLLELD
Sequence in bold is th IgE leader sequence; underlined
sequence is the linker sequence; double underlined
amino acids are glycan mutations.
TRO11_MD39_L14G8_gp120
QGQLWVTVYYGVPVWKDASTTLFCASDAKAYDTE V HNVWATHACVPTDP
NPQEVVLGNVTENFNMWKNNMVDQMHEDIISLWDQSLKPCVKLTPLCVTLNCTDNI
TNTNTNSSKNSSTHSYNNSLEGEMKNCSFNITAGIRDKVKKEYALFYKLDVVPIEED
KDTNKTTYRLRSCNTSVITQACPKVTFEPIPIHYCATAGFAILKCNDKKFNGTGPCTN
VSTVQCTHGIRPVVSTQLLLNGSLAEEEVIIRSENFTNNAKTIIVQLNESIAINCTRPNN
NTVRSIHIGPGRAFYYTGDIIGDIRQAHCNISRTEWNSTLRQIVTKLREQLGDPNKTIIF
AQSSGGDTEITMHSFNCGGEFFYCNTTKLFNSTWNGNNTTESDSTGENITLPCRIKQII
NLWQEVGKAMYAPPIKGQISCSSNITGLLLTRDGGNNNSSGPETFRPGGGNMKDNW
RSELYKYKVIKIEPLGVAPTRCKRRVV
TRO11_MD39_L14G8_gp41
AVGTLGAMSLGFLGAAGSTMGAASVTLTVQARLLLSGIVQQQNNLLRAPEP
QQHMLQDTHWGIKQLQARVLAVEHYLRDQQLLGIWGCSGKLICCTNVPWNASWSN
KSLNNIWENMTWMNWSREIDNYTDLIYILLEKSQIQQEKNNQSLLELD
Bolded residues are glycans
X2278_FJ817366_MD39_L14G8
MDWTWILFLVAAATRVHSTNNLWVTVYYGVPVWKEATTTLFCASEAKAY
DTEVHNIWATHACVPTDPNPQEMELKNVTENFNMWKNNMVEQMHEDIISLWDQSL
KPCVKLTPLCVTLDCTNINSTNSTNNTSSNSKMEETIGVIKNCSFNVTTNIRDKVKKE
NALFYSLDLVSIGNSNTSYRLISCNTSIITQACPKVSFDPIPIHYCAPAGFAILKCRDKKF
NGTGPCRNVSSVQCTHGIRPVVSTQLLLNGSLAEEEIIIRSANLTDNAKTIIIQLNETIQI
NCTRPNNNTVRSIPIGPGRTFYYTGDIIGDIRKAYCNISATKWNNTLRQIAEKLREKFN
KTIIFAQSSGGDPEVVRHTFNCGGEFFYCNSSQLFNSTWYSNGTSNGGLNNSANITLP
CRIKQIINLWQEVGKAMYAPPIKGVINCLSNITGIILTRDGGENNGTTETFRPGGGDM
RDNWRSELYKYKVVIEPLGIAPTKCKRRVVGSHSGSGGSGSGGHAAVGLGAVSLG
FLGLAGSTMGAASVTLTVQARLLLSGIVQQQNNLLRAPEPQQQLLQDTHWGIKQLQ
ARVLALEHYLKDQQLLGIWGCSGKLICCTTVPWNASWSNKSYNQIWNNMTWMNW
SREIDNYTNLIYNLIEESQSQQEKNNLSLLQLD
Sequence in bold is the IgE leader sequence; underlined
sequence is the linker sequence; double underlined
amino acids comprise glycan mutations.
X22798_MD39_L14G8_gp120
TNNLWVTVYYGVPVWKEATTTLFCASEAKAYDTEVHNIWATHACVPTDPNP
QEMELKNVTENFNMWKNNMVEQMHEDIISLWDQSLKPCVKLTPLCVTLDCTNINST
NSTNNTSSNSKMEETIGVIKNCSFNVTTNIRDKVKKENALFYSLDLVSIGNSNTSYRLI
SCNTSIITQACPKVSFDPIPIHYCAPAGFAILKCRDKKFNGTGPCRNVSSVQCTHGIRPV
VSTQLLLNGSLAEEEIIIRSANLTDNAKTIIIQLNETIQINCTRPNNNTVRSIPIGPGRTFY
YTGDIIGDIRKAYCNISATKWNNTLRQIAEKLREKFNKTIIFAQSSGGDPEVVRHTFNC
GGEFFYCNSSQLFNSTWYSNGTSNGGLNNSANITLPCRIKQIINLWQEVGKAMYAPPI
KGVINCLSNITGIILTRDGGENNGTTETFRPGGGDMRDNWRSELYKYKVVKIEPLGIA
PTKCKRRVV
X22798_MD39_L14G8_gp41
AVGLGAVSLGFLGLAGSTMGAAVTLTVQARLLLSGIVQQQNNLLRAPEPQQ
QLLQDTHWGIKQLQARVLALEHYLKDQQLLGIWGCSGKLICCTTVPWNASWSNKS
YNQIWNNMTWMNWSREIDNYTNLIYNLIEESQSQQEKNNLSLLQLD
double underlined amino acid are glycan mutations
398F1_HM215312_MD39_L14G8
MDWTWILFLVAAATRVHSMGNLWVTVYYGVPVWKDAETTLFCASDAKA
YHTEVHNVWATHACVPTDPNPQEINLENVTEEFNMWKNKMVEQMHEDIISLWDQS
LKPCVQLTPLCVTLDCQYNVTNINSTSDMAREINNCSYNITTELRDREQKVYSLFYRS
DIVQMNSDNSSKYRLINCNTSAIKQACPKVTFEPIPIHYCAPAGFAILKCKDKEFNGTG
PCKNVSTVQCTHGIKPVVSTQLLLNGSLAEEKVIIRSENITDNAKNIIVQLKEPVKINC
TRPNNNTVKSVRIGPGQTFYYTGEIIGDIRQAHCNVSKAHWENTLQEVANQLKLMIH
SNKTIIFANSSGGDLEITTHSFNCGGEFFYCYTSGLFNYTFNDTSTNSTESKSNDTITLQ
CRIKQIINMWQRAGQAVYAPPIPGIIRCESNITGLILTRDGGNNNSNTNETFRPGGGDM
RDNWRSELYRYKVVKIEPIGVAPTTCKRRVVGSHSGSGGSGSGGHAVVGIGAVSLGF
LGAAGSTMGAASITLTVQARQLLSGIVQQQSNLLRAPEPQQHLLKDTHWGIKQLKA
RVLAVEHYLKDQQLLGIWGCSGKLICCTNVPWNSSWSNKSLGEIWDNMTWLNWSK
EIENYTQIIYELIEESQNQQEKNNQSLLALD
Sequence in bold is the IgE leader sequence; underlined
sequence is the linker sequence; double underlined
amino acids are glycan mutations.
MGNLWVTVYYGVPVWKDAETTLFCASDAKAYHTEVHNVWATHACVPTDP
NPQEINLENVTEEFNMWKNKMVEQMHEDIISLWDQSLKPCVQLTPLCVTLDCQYNV
TNINSTSDMAREINNCSYNITTELRDREQKVYSLFYRSDIVQMNSDNSSKYRLINCNT
SAIKQACPKVTFEPIPIHYCAPAGFAILKCKDKEFNGTGPCKNVSTVQCTHGIKPVVST
QLLLNGSLAEEKVIIRSENITDNAKNIIVQLKEPVKINCTRPNNNTVKSVRIGPGQTFY
YTGEIIGDIRQAHCNVSKAHWENTLQEVANQLKLMIHSNKTIIFANSSGGDLEITTHSF
NCGGEFFYCYTSGLFNYTFNDTSTNSTESKSNDTITLQCRIKQIINMWQRAGQAVYAP
PIPGIIRCESNITGLILTRDGGNNNSNTNETFRPGGGDMRDNWRSELYRYKVVKIEPIG
VAPTTCKRRVV
AVGIGAVSLFLGAAGSTGAASITLTVQARQLLSGIVQQQSNLLRAPEPQQH
LLKDTHWGIKQLKARVLAVEHYLKDQQLLGIWGCSGKLICCTNVPWNSSWSNKSLG
EIWDNMTWLNWSKEIENYTQIIYELIEESQNQQEKNNQSLLALD
double underlined amino acids are glycan mutations
246F3_HM215279_MD39_L14G8
MDWTWILFLVAAATRVHSMQDLWVTVYYGVPVWKDAKTTLFCASDAKA
YEKEVHNVWATHACVPTDPNPQEIVMANVTEEFNMWKNNMVEQMHEDIISLWDQS
LKPCVKLTPLCVTLDCKDYNYSITNNSTGMEGEIKNCSYNITTELRDKRQKVYSLFY
RLDVVQINDSNDRNNSQYRLINCNTTTMTQACPKVTFDPIPIHYCAPAGFAILKCNNK
TFNGKGPCNNVSSVQCTHGIKPVVSTQLLLNGSLAEKEIIIRSENLTDNVKTIIVHLNE
SVEINCTRPNNNTVKSVRIGPGQTFYYTGDIIGNIRQAHCTVNKTEWNTALTRVSKKL
KEYFPNKTIAFQPSSGGDLEITTFSFNCRGEFFYCNTSDLFNGTFNETSGQFNSTFNSTL
QCRIKQIINMWQEVGQAMYAPPIAGSITCISNITGLILTRDGGNTNSTKETFRPGGGN
MRDNWRSELYKYKVVKIEPLGVAPTKCRRRVVGSHSGSGGSGSGGHAAVGIGAVSI
GFLGAAGSTMGAASITLTVQARQLLSGIVQQQSNLLRAPEPQQHLLKDTHWGIKQLQ
ARVLAVEHYLKDQQLLGIWGCSGKLICCTNVPWNSSWSNKSQDEIWDNMTWLNWS
KEISNYTQIIYNLIEESQTQQELNNRSLLALD
Sequence in bold is the IgE leader sequence; underlined
sequence is the linker sequence; double underlined
amino acids are glycan mutations
MQDLWVTVYYGVPVWKDAKTTLFCASDAKAYEKEVHNVWATHACVPTDP
NPQEIVMANVTEEFNMWKNNMVEQMHEDIISLWDQSLKPCVKLTPLCVTLDCKDY
NYSITNNSTGMEGEIKNCSYNITTELRDKRQKVYSLFYRLDVVQINDSNDRNNSQYR
LINCNTTTMTQACPKVTFDPIPIHYCAPAGFAILKCNNKTFNGKGPCNNVSSVQCTHG
IKPVVSTQLLLNGSLAEKEIIIRSENLTDNVKTIIVHLNESVEINCTRPNNNTVKSVRIGP
GQTFYYTGDIIGNIRQAHCTVNKTEWNTALTRVSKKLKEYFPNKTIAFQPSSGGDLEI
TTFSFNCRGEFFYCNTSDLEFNGTFNETSGQFNSTFNSTLQCRIKQIINMWQEVGQAMY
APPIAGSITCISNITGLILTRDGGNTNSTKETFRPGGGNMRDNWRSELYKYKVVKIEPL
GVAPTKCRRRVV
AVGIGAVSIGFLGAAGSTMGAASITLTVQARQLLSGIVQQQSNLLRAPEPQQH
LLKDTHWGIKQLQARVLAVEHYLKDQQLLGIWGCSGKLICCTNVPWNSSWSNKSQ
DEIWDNMTWLNWSKEISNYTQIIYNLIEESQTQQELNNRSLLALD
double underlined amino acids are glcan mutations
CE0217_FJ443575_MD39_L14G8
MDWTWILFLVAAATRVHSAKDMWVTVYYGVPVWREAKTTLFCASDAKA
YEREVHNVWATHACVPTDPNPQERVLENVTENFNMWKNNMVDQMHEDIISLWDEA
LKPCIKLTPLCVTLNCGNAIVNESTIEGMKNCSFNVTTELKDKKKKEYALFYKLDVV
PLNGENNNSNKNFSEYRLINCNTSTITQACPKVSFDPIPIHYCAPAGFAILKCNNETF
NGTGPCNNVSTVQCTHGIKPVVSTQLLLNGSLAEKEIIIRSENLTNNAKIIIVHLNNPV
KIICTRPGNNTVKSMRIGPGQTFYYTGDIIGDIRRAYCNISEKTWYDTLKNVSDKFQE
HFPNASIEFKPSAGGDLEITTHSFNCRGEFFYCDTSELFNGTYNNSTYNSSNNITLQCKI
KQIINMWQGVGRAMYAPPIAGNITCESNITGLLLTRDGGNNKSTPETFRPGGGDMRD
NWRSELYKYKVVEIKPLGIAPTKCKRRVVGSHSGSGGSGSGGHAAVGMGAVSLGFL
GAAGSTMGAASLTLTVQARQLLSGIVQQQNNLLRAPEPQQHMLQDTHWGIKQLQA
RVLAIEHYLTDQQLLGIWGCSGKLICCTNVPWNNSWSSNKSSYEDIWGRNMTWMNWS
REINNYTNTIYRLLIKSSQNQQEKNNKSLLELD
Sequence in bold is the IgE leader sequence; underlined
sequence is the linker sequence; double underlined
amino acids are glycan mutations
AKDMWVTVYYGVPVWREAKTTLFCASDAKAYEREVHNVWATHACVPTDP
NPQERVLENVTENFNMWKNNMVDQMHEDIISSLWDESLKPCIKLTPLCVTLNCGNAI
VNESTIEGMKNCSFNVTTELKDKKKKEYALFYKLDVVPLNGENNNSNSKNFSEYRLI
NCNTSTITQACPKVSFDPIPIHYCAPAGFAILKCNNETFNGTGPCNNVSTVQCTHGIKP
VVSTQLLLNGSLAEKEIIIRSENLTNNAKIIIVHLNNPVKIICTRPGNNTVKSMRIGPGQ
TFYYTGDIIGDIRRAYCNISEKTWYDTLKNVSDKFQEHFPNASIEFKPSAGGDLEITTH
SFNCRGEFFYCDTSELFNGTYNNSTYNSSNNITLQCKIKQIINMWQGVGRAMYAPPIA
GNITCESNITGLLLTRDGGNNKSTPETFRPGGGDMRDNWRSELYKYKVVEIKPLGIAP
TKCKRRVV
AVGMGAVSLGFLGAAGSTMGASLTLTVQARQLLSGIVQQQNNLLRAPEPQ
QHMLQDTHWGIKQLQARVLAIEHYLTDQQLLGIWGCSGKLICCTNVPWNNSWSNK
SYEDIWGRNMTWMNWSREINNYTNTIYRLLIKSQNQQEKNNKSLLELD
double underlined amino acids are glycan mutations
C31176_FJ444437_MD39_L14G8
MDWTWILFLVAAATRVHIVGNLWVTVYYGVPVWKEAKTTLFCASDAKAY
EKEVHNVWATHACVPTDPNPQEMVLENVTENFNMWKNDMVDQMHEDVISLWDQ
SLKPCVKLTPLCVTLTCTNTTVSNGSSNSNANFEEMKNCSFNATTEIKDKKKNEYAL
FYKLDIVPLNNSSGKYRLINCNTSAIAQACPKVTFEPIPIHYCAPAGYAILKCNNKTFN
GTGPCNNVSTVQCTHGIKPVVSTQLLLNGSLAEKEIIIRSENLTNNAKTIIIHLNESVGI
VCTRPSNNTVKSIRIGPGQTFYYTGDIIGDIRQAHCNVSKQNWNRTLQQVGRKLAEH
FPNRNITFAHSSGGDLEITTHSFNCRGEFFYCNTSGLGNGTYHPNGTYNETAVNSSDTI
TLQCRIKQIINMWQEVGRAMYAPPIAGNITCNSTITGLLLTRDGGINQTGEEIFRPGGG
DMRDNWRNELYKYKVVEIKPLGIAPTKCKRRVVGSHSGSGGSGSGGHAAVGIGAVS
LGFLGAAGSTMGAASITLTVQARQLLSGIVQQQSNLLRAPEPQQHMLQDTHQGIKQ
LQARVLAIEHYLKDQQLLGIWGCSGKLICCTNVPWNSSWSNRSQEDIWNNMTWMN
WSREIDNYTHTIYSLLEESQIQQEKNNKSLLALD
Sequence in bold is the IgE leader sequence; underlined
sequence is the linker sequence; double underlined
amino acids are glycan mutations
VGNLWVTVYYGVPVWKEAKTTLFCASDAKAYEKEVHNVWATHACVPTDP
NPQEMVLENVTENFNMWKNDMVDQMHEDVISLWDQSLKPCVKLTPLCVTLTCTNT
TVSNGSSNSNANFEEMKNCSFNATTEIKDKKKNEYALFYKLDIVPLNNSSGKYRLIN
CNTSIAQACPKVTFEPIPIHYCAPAGYAILKCNNKTFNGTGPCNNVSTVQCTHGIKP
VVSTQLLLNGSLAEKEIIIRSENLTNNAKTIIIHLNESVGIVCTRPSNNTVKSIRIGPGQT
FYYTGDIIGDIRQAHCNVSKQNWNRTLQQVGRKLAEHFPNRNITFAHSSGGDLEITTH
SFNCRGEFFYCNTSGLFNGTYHPNGTYNETAVNSSDTITLQCRIKQIINMWQEVGRA
MYAPPIAGNITCNSTITGLLLTRDGGINQTGEEIFRPGGGDMRDNWRNELYKYKVVEI
KPLGIAPTKCKRRVV
AVGIGAVSLGFLGAAGSTMGAASITLTVQARQLLSGIVQQQSNLLRAPEQQH
MLQDTHWGIKQLQARVLAIEHYLKDQQLLGIWGCSGKLICCTNVPWNSSWSNRSQE
DIWNNMTWMNWSREIDNYTHTIYSLLEESQIQQEKNNKSLLALD
double underlined amino acids are glycan mutations
25710_EF117271_MD39_L14G8
MDWTWILFLVAAATRVHSGGNLWVTVYYGVPVWKEATTTLFCASDAKAY
DKEVHNVWATHACVPTDPNPQEMVLGNVTENFNMWKNEMVNQMHEDVISLWDQ
SLKPCVKLTPLCVTLECSNVTYNESMKEVKNCSFNLTTELRDKKQKVHALFYRLDIV
PLNDTEKKNSSRPYRLINCNTSAITQACPKVTFDPIPIHYCTPAGYAILKCNDKKFNGT
GPCHKVSTVQCTHGIKPVVSTQLLLNGSLAEGEIIIRSENLTNNAKTIIVHLNQSVEIVC
ARPSNNTVTSIRIGPGQTFYYTGAITGDIRQAHCNISKDKWNETLQRVGEKLAEHFPN
KTIKFASSSGGDLEITTHSFNCRGEFFYCNTSGLFNGTFNGTYVSPNSTDSNSSSIITIPC
RIKQIINMWQEVGRAMYAPPIAGNITCKSNITGLLLVRDGGTGSESNKTEIFRPGGGD
MRDNWRSELYKYKVVEIKPLGVAPTKCKRRVVGSHSGSGGSGSGGHAAVGIGAVSL
GFLGAAGSTMGAASITLTVQARQLLSGIVQQQSNLLRAPEPQQHLLQDTHWGIKQLQ
TRVLAIEHYLKDQQLLGIWGCSGKLICCTAVPWNYSWSNRSQDDIWDNMTWMNWS
KEISNYTNTIYKLLEDSQIQQEKNNKSLLALD
Sequence in bold is the IgE leader sequence; underlined
sequence is the linker sequence; double underlined
amino acids are glycan mutations
GGNLWVTVYYGVPVWKEATTTLFCASDAKAYDKEVHNVWATHACVPTDP
NPQEMVLGNVTENFNMWKNEMVNQMHEDISLWDQSLKPCVKLTPLCVTLECSNV
TYNESMKEVKNCSFNLTTELRDKKQKVHALFYRLDIVPLNDTEKKNSSRPYRLINCN
TSAITQACPKVTFDPIPIHYCTPAGYAILKCNDKKFNGTGPCHKVSTVQCTHGIKPVV
STQLLLNGSLAEGEIIIRSENLTNNAKTIIVHLNQSVEIVCARPSNNTVTSIRIGPGQTFY
YTGAITGDIRQAHCNISKCKWNETLQRVGEKLAEHFPNKTIKFASSSGGDLEITTHSF
NCRGEFFYCNTSGLFNGTFNGTYVSPNSTDSNSSSIITIPCRIKQIINMWQEVGRAMYA
PIIAGNITCKSNITGLLLVRDGGTGSESNKTEIFRPGGGDMRDNWRSELYKYKVVEIK
PLGVAPTKCKRRVV
AVGIGAVSLGFLGAAGSTMGAASITLTVQARQLLSGIVQQQSNLLRAPEPQQH
LLQDTHWGIKQLQTRVLAIEHYLKDQQLLGIWGCSGKLICCTAVPWNYSWSNRSQD
DIWDNMTWMNWSKEISNYTNTIYKLLEDSQIQQEKNNKSLLALD
double underlined amino acids are glycan mutations
BJOX2000_HM215364_MD39_L14G8
MDWTWILFLVAAATRVHIVGNLWVTVYYGVPVWKEATTTLFCASDAKAY
DTEVHNVWATHACVPTDPDPQEMFLENVTENFNMWKNNMVDQMHEDVISLWDQS
LKPCVKLTPLCVTLECKNVNSSSSDTKNGTDPEMKNCSFNATTELRDRKQKVYALF
YKLDIVPLNEKNSSEYRLINCNTSTITQACPKVTFDPIPIHYCTPAGYAILKCNDEKFN
GTGPCSNVSTVQCTHGIKPVVSTQLLLNGSLAEKGIIIRSENLTNNVKTIIVHLNQSVEI
LCIRPNNNTVKSIRIGPGQTFYYTGEIIGDIRQAHCNISGKVWNETLQRVGEKLAEYFP
NKTIKFASSSGGDLEITTHSFNCGGEFFYCNTSKLFNGTFNGTYMPNVTEGNSTISIPC
RIKQIINMWQKVGRAMYAPPIEGNITCKSKITGLLLERDGGPENDTEIFRPGGGDMRN
NWRSELYKYKVVEIKPLGVAPTECKRRVVGSHSGSGGSGSGGHAAVGIGAVSLGFL
GVAGSTMGAASMALTVQARQLLSGIVQQQSNLLRAPEPQQHLLQDTHWGIKQLQTR
VLAIEHYLKDQQLLGIWGCSGKLICCTAVPWNSSWSNKSQEEIWENMTWMNWSKEI
SNYTDTIYRLLEDSQNQQERNNKSLLALD
Sequence in bold is the IgE leader sequence; underlined
sequence is the linker sequence; double underlined
amino acids are glycan mutations
VGNLWVTVYYGVPVWKEATTLFCASDAKAYDTEVHNVWATHACVPTDPD
PQEMFLENVTENFNMWKNNMVDQMHEDVISLWDQSLKPCVKLTPLCVTLECKNVN
SSSSDTKNGTDPEMKNCSFNATTELRDRKQKVYALFYKLDIVPLNEKNSSEYRLINC
NTSTITQACPKVTFDPIPIHYCTPAGYAILKCNDEKFNGTGPCSNVSTVQCTHGIKPVV
STQLLLNGSLAEKGIIIRSENLTNNVKTIIVHLNQSVEILCIRPNNNTVKSIRIGPGQTFY
YTGEIIGDIRQAHCNISGKVWNETLQRVGEKLAEYFPNKTIKFASSSGGDLEITTHSFN
CGGEFFYCNTSKLFNGTFNGTYMPNVTEGNSTISIPCRIKQIINMWQKVGRAMYAPPI
EGNITCKSKITGLLLERDGGPENDTEIFRPGGGDMRNNWRSELYKYKVVEIKPLGVA
PTECKRRVV
AVGIGAVSLGFLGVAGSTMGAASMALTVQARQLLSGIVQQQSNLLRAPEPQQ
HLLQDTHWGIKQLQTRVLAIEHYLKDQQLLGIWGCSGKLICCTAVPWNSSWSNKSQ
EEIWENMTWMNWSKEISNYTDTIYRLLEDSQNQQERNNKSLLALD
double underlined amino acids are glycan mutations
CH119_EF117261_MD39_L14G8
MDWTWILFLVAAATRVHIVGNLWVTVYYGVPVWKEATTTLFCASDAKAY
DTEVHNVWATHACVPTDPSPQELVLENVTENFNMWKNEMVNQMHEDVISLWDQS
LKPCVKLTPLCVTLECSKVSNNETDKYNGTEEMKNCSFNATTVVRDRQQKVYALFY
RLDIVPLTEKNSSENSSKYYRLINCNTSAITQACPKVSFEPIPIHYCTPAGYAILKCNDK
TFNGTGPCHNVSTVQCTHGIKPVVSTQLLLNGSLAEGEIIIRSENLTNNVKTILVHLNQ
SVEIVCTRPNNNTVKSIRIGPGQTFYYTGDIIGDIRQAHCNISKWHETLKRVSEKLAEH
FPNKTINFTSSSGGDLEITTHSFTCRGEFFYCNTSGLFNSTYMPNGTYLHGDTNSNSSI
TIPCRIKQIINMWQEVGRAMYAPPIEGNITCKSNITGLLLVRDGGTESNNTETNNTEIF
RPGGGDMRDNWRSELYKYKVVEIKPLGVAPTACKRRVVGSHSGSGGSGSGGHAAV
GIGAVSLGFLGVAGSTMGAASMTLTVQARQLLSGIVQQQSNLLRAPEPQQHLLQDT
HWGIKQLQTRVLAIEHYLKDQQLLGIWGCSGKLICCTAVPWNSSWSNKSQKEIWDN
MTWMNWSKEISNYTNTIYKLLEDSQNQQESNNKSLLALD
Sequence in bold is the IgE leader sequence; underlined
sequence is the linker sequence; double underlined
amino acids are glycan mutations
VGNLWVTVYYGVPVWKEATTTLFCASDAKAYDTEVHNVWATHACVPTDPS
PQELVLENVTENFNMWKNEMVNQMHEDVISLWDQSLKPCVKLTPLCVTLECSKVS
NNETDKYNGTEEMKNCSFNATTVVRDRQQKVYALFYRLDIVPLTEKNSSENSSKYY
RLINCNTSAITQACPKVSFEPIPIHYCTPAGYAILKCNDKTFNGTGPCHNVSTVQCTHG
IKPVVSTQLLLNGSLAEGEIIIRSENLTNNVKTILVHLNQSVEIVCTRPNNNTVKSIRIGP
GQTFYYTGDIIGDIRQAHCNISKWHETLKRVSEKLAEHFPNKTINFTSSSGGDLEITTH
SFTCRGEFFYCNTSGLFNSTYMPNGTYLHGDTNSNSSITIPCRIKQIINMWQEVGRAM
YAPPIEGNITCKSNITGLLLVRDGGTESNNTETNNTEIFRPGGGDMRDNWRSELYKYK
VVEIKPLGVAPTACKRRVV
AVGIGAVSLGFLGVAGSTMGAASMTLTVQARQLLSGIVQQQSNLLRAPEPQQ
HLLQDTHWGIKQLQTRVLAIEHYLKDQQLLGIWGCSGKLICCTAVPWNSSWSNKSQ
KEIWDNMTWMNWSKEISNYTNTIYKLLEDSQNQQESNNKSLLALD
double underlined amino acids are glycan mutations
X1632_FJ817370_MD39_L14G8
SEQ ID NO:
MDWTWILFLVAAATRVHSSNNLWVTVYYGVPVWEDADTTLFCASDAKAY
STESHNVWATHACVPTDPNPQEIYLENVTEDFNMWENNMVEQMQEDIISLWDESLK
PCVKLTPLCVTLTCTNVTNVTDSVGTNSRLKGYKEELKNCSFNTTTEIRDKKKQEYA
LFYKLDIVPINDNSNNSNGYRLINCNVSTIKQACPKVSFDPIPIHYCAPAGFAILKCRD
KEFNGTGTCRNVSTVQCTHGIKPVVSTQLLLNGSLAEGDIIIRSENITDNAKTIIVHLN
KTVSITCTRPNNNTVKSIRIGPGQALYYTGAIIGDTRQAHCNINGSEWYEMIQNVKNK
LNETFKKNITFAPSSGGDLEITTHSFNCRGEFFYCNTSELFNSSHLFNGSTLSTNGTITL
PCRIKQIVRMWQRVGQAMYAPPIAGNITCRSNITGLLLTRDGGTNKDTNEAETFRPG
GGDMRDNWRSELYKYKVVKIKPLGVAPTRCRRRVVGSHSGSGGSGSGGHAAIGLG
TVSLGFLGTAGSTMGAASITLTVQVRQLLSGIVQQQSNLLRAPEPQQHLLQDTHWGI
KQLQARVLAVEHYLKDQQILGIWGCSGKLICCTNVPWNSSWSNKSYSDIWDNLTWI
NWSREISNYTQQIYTLLEESQNQQEKNNQSLLALD
Sequence in bold is the IgE leader sequence;
underlined sequence is the linker sequence; double
underlined amino acids are glycan mutations
SNNLWVTVYYGVPVWEDADTTLFCASDAKAYSTESHNVWATHACVPTDPN
PQEIYLENVTEDFNMWENNMVEQMQEDIISLWDESLKPCVKLTPLCVTLTCTNVTN
VTDSVGTNSRLKGYKEELKNCSFNTTTEIRDKKKQEYALFYKLDIVPINDNSNNSNG
YRLINCNVSTIKQACPKVSFDPIPIHYCAPAGFAILKCRDKEFNGTGTCRNVSTVQCTH
GIKPVVSTQLLLNGSLAEGDIIIRSENITDNAKTIIVHLNKTVSITCTRPNNNTVKSIRIG
PGQALYYTGAIIGDTRQAHCNINGSEWYEMIQNVKNKLNETFKKNITFAPSSGGDLEI
TTHSFNCRGEFFYCNTSELFNSSHLFNGSTLSTNGTITLPCRIKQIVRMWQRVGQAMY
APPIAGNITCRSNITGLLLTRDGGTNKDTNEAETFRPGGGDMRDNWRSELYKYKVVK
IKPLGVAPTRCRRRVV
AIGLGTVSLGFLGTAGSTMGAASITLTVQVRQLLSGIVQQQSNLLRAPEPQQH
LLQDTHWGIKQLQARVLAVEHYLKDQQILGIWGCSGKLICCTNVPWNSSWSNKYS
DIWDNLTWINWSREISNYTQQIYTLLEESQNQQEKNNQSLLALD
double underlined amino acids are glycan mutations
CNE8_HM215427_MD39_L14G8
MDWTWILFLVAAATRVHSSDNLWVTVYYGVPVWRDADTTLFCASDAKAY
DTEVHNVWATHACVPTDPNPQEIHLENVTENFNMWKNKMAEQMQEDVISLWDESL
KPCVQLTPLCVTLNCTNANLNATVNASTTIGNITDEVRNCSFNTTTELRDKKQNVYA
LFYKLDIVPINNNSEYRLINCNTSVIKQACPKVSFDPIPIHYCAPAGYAILRCNDKNFN
GTGPCKNVSSVQCTHGIKPVVSTQLLLNGSLAEDEIIIRSENLTDNVKTIIVHLNKSVEI
NCTRPSNNTVTSVRIGPGQVFYYTGDIIGDIRKAYCEINRTKWHETLKQVATKLREHF
NKTIIFQPPSGGDIEITMHHFNCRGEFFYCNTTKLFNSTWGENTTMEGHNDTIVLPCRI
KQIVNMWQGVGQAMYAPPIRGSINCVSNITGILLTRDGGTNMSNETFRPGGGNIKDN
WRSELYKYKVVEIEPLGIAPTKCKRRVVGSHSGSGGSGSGGHAAVGIGAMSFGFLGA
AGSTMGAASITLTVQARQLLSGIVQQQSNLLRAPEPQQHLLQDTHWGIKQLQARVL
AVEHYLKDQKFLGLWGCSGKIICCTAVPWNSTWSNRSYEEIWDNMTWINWSREISN
YTSQIYEILTESQNQQDRNNKSLLELD
Sequence in bold is the IgE leader sequence;
underlined sequence is the linker sequence; double
underlined amino acids are glycan mutations
SDNLWVTVYYGVPVWRDADTTLFCASDAKAYDTEVHNVWATHACVPTDPN
PQEIHLENVTENFNMWKNKMAEQMQEDVISLWDESLKPCVQLTPLCVTLNCTNANL
NATVNASTTIGNITDEVRNCSFNTTTELRDKKQNVYALFYKLDIVPINNNSEYRLINC
NTSVIKQACPKVSFDPIPIHYCAPAGYAILRCNDKNFNGTGPCKNVSSVQCTHGIKPV
VSTQLLLNGSLAEDEIIIRSENLTDNVKTIIVHLNKSVEINCTRPSNNTVTSVRIGPGQV
FYYTGDIIGDIRKAYCEINRTKWHETLKQVATKLREHFNKTIIFQPPSGGDIEITMHHF
NCRGEFFYCNTTKLFNSTWGENTTMEGHNDTIVLPCRIKQIVNMWQGVGQAMYAPP
IRGSINCVSNITGILLTRDGGTNMSNETFRPGGGNIKDNWRSELYKYKVVEIEPLGIAP
TKCKRRVV
AVGIGAMSFGFLGAAGSTMGAASITLTVQARQLLSGIVQQQSNLLRAPEPQQ
HLLQDTHWGIKQLQARVLAVEHYLKDQKFLGLWGCSGKIICCTAVPWNSTWSNRS
YEEIWDNMTWINWSREISNYTSQIYEILTESQNQQDRNNKSLLEDL
double underlined amino acids are glycan mutations
CNE55_HM215418_MD39_L14G8
MDWTWILFLVAAATRVHSSDKLWVTVVYGVPVWRDADTTLFCASDAKAH
ETEVHNVWATHACVPTDPNPQEIHLVNVTENFNMWKNKMVEQMQEDVISLWDESL
KPCVKLTPLCVTLNCTTANTNETKNNTTDDNIKDEMKNCTFNMTTEIRDKKQRVSA
LFYKLDIVPIDDSKNNSEYRLINCNTSVIKQACPKVSFDPIPIHYCTPAGYVILKCNDK
NFNGTGPCKNVSSVQCTHGIKPVVSTQLLLNGSLAEEEIIIRSENLTDNAKNIIVHLNK
SVEINCTRPSNNTVTSVRIGPGQVFYYTGDITGDIRKAYCEIDGTEWNKTLTQVAEKL
KEHFNKTIVYQPPSGGDLEITMHHFNCRGEFFYCNTTQLFNNSVGNSTIKLPCRIKQII
NMWQGVGQAMYAPPISGAINCLSNITGILLTRDGGGNNRSNETFRPGGGNIKDNWRS
ELYKYKVVEIEPLGIAPTKCKRRVVGSHSGSGGSGSGGHAAVGIGAMSFGFLGAAGS
TMGAASITLTVQARQLLSGIVQQQSNLLRAPEPQQHMLQDTHWGIKQLQARVLAVE
HYLKDQRFLGLWGCSGKTICCTAVPWNSTWSNKTYEEIWDNMTWTNWSREISNYT
NQIYSILTESQSQQDKNNKSLLELD
Sequence in bold is the IgE leader sequence; underlined
sequence is the linker sequence; double underlined
amino acids are glycan mutations
SDKLWVTVYYGVPVWRDADTTLFCASDAKAHETEVHNVWATHACVPTDPN
PQEIHLVNVTENFNMWKNKMVEQMQEDVISLWDESLKPCVKLTPLCVTLNCTTANT
NETKNNTTDDNIKDEMKNCTFNMTTEIRDKKQRVSALFYKLDIVPIDDSKNNSEYRLI
NCNTSVIKQACPKVSFDPIPIHYCTPAGYVILKCNDKNFNGTGPCKNVSSVQCTHGIK
PVVSTQLLLNGSLAEEEIIIRSENLTDNAKNIIVHLNKSVEINCTRPSNNTVTSVRIGPG
QVFYYTGDITGDIRKAYCEIDGTEWNKTLTQVAEKLKEHFNKTIVYQPPSGGDLEIT
MHHFNCRGEFFYCNTTQLFNNSVGNSTIKLPCRIKQIINMWQGVGQAMYAPPISGAI
NCLSNITGILLTRDGGGNNRSNETFRPGGGNIKDNWRSELYKYKVVEIEPLGIAPTKC
KRRVV
AVGIGAMSFGFLGAAGSTMGAASITLTVQARQLLSGIVQQQSNLLRAPEPQQ
HMLQDTHWGIKQLQARVLAVEHYLKDQRFLGLWGCSGKTICCTAVPWNSTWSNKT
YEEIWDNMTWTNWSREISNYTNQIYSILTESQSQQDKNNKSLLELD
double underlined amino acids are glycan mutations
AD8_MD64_link14_TS1
MDWTWILFLVAAATRVHIVENLWVTVYYGVPVWKEATTTLFCASDAKAY
DTEVHNVEATHECVPTDPNPQEVVLENVTENFNMWKNNMVEQMHEDIIELWDQSL
KPCVKLTPLCVTLNCTDLRNVTNINNSSEGMRGEIKNCSFNITTSIRDKVKKDYALFY
RLDVVPIDNDNTSYRLINCNTSTITQACPKVSFEPIPIHYCTPAGFAILKCKDKKFNGT
GPCKNVSTVQCTHGIRPVVSTQLLLNGSLAEEEVIIRSSNFTDNAKNIIVQLKKESVEIN
CTRPNNNTVKSIHIGPGRAFYYTGDIIGDIRQAHCNISRTKWNNTLNQIATKLKEQFG
NNKTIVFNQSSGGDPEIVMHSFNCGGEFFYCNSTQLFNSTWNFNGTWNLTQSNGTEG
NDTITLPCRIKQIINMWQEVGKAMYAPPIRGQIRCSSNITGLILTRDGGNNHNNDTETF
RPGGGDMRDNWRSELYKYKVVKIEPLGVAPTKCKRRVVQSHSGSGGSGSGGHAAV
GTIGAMSLGFLGAAGSTMGAASITLTVQARLLLSGIVQQQNNLLRAPEPQQHLLQLT
VWGIKQLQARVLAVEHYLRDQQLLGIWGCSGKLICCTAVPWNASWSNKTLDMIWN

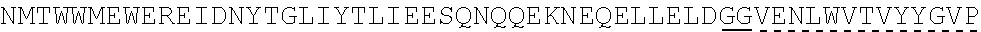

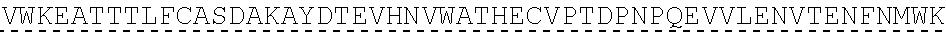

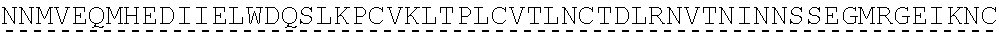

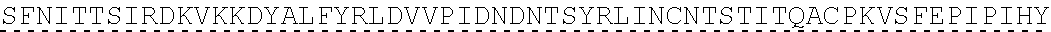

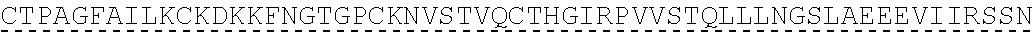

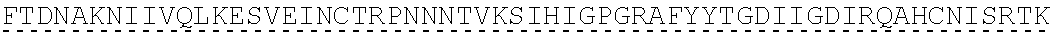

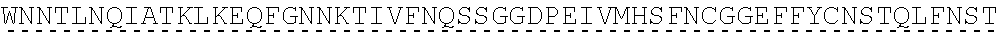

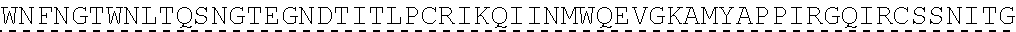

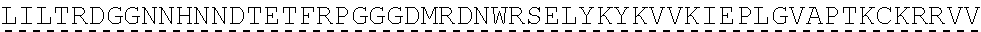

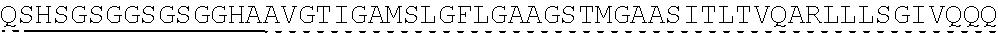

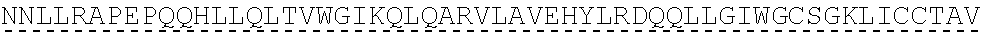

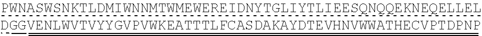

QEVVLENVTENFNMWKNNMVEQMHEDIIELWDQSLKPCVKLTPLCVTLNCTDLRN
VTNINNSSEGMRGEIKNCSFNITTSIRDKVKKDYALFYRLDVVPIDNDNTSYRLINCN
TSTITQACPKVSFEPIPIHYCTPAGFAILKCKDKKFNGTGPCKNVSTVQCTHGIRPVVS
TQLLLNGSLAEEEVIIRSSNFTDNAKNIIVQLKESVEINCTRPNNTVKSIHIGPGRAFY
YTGDIIGDIRQAHCNISRTKWNNTLNQIATKLKEQFGNNKTIVFNQSSGGDPEIVMHS
FNCGGEFFYCNSTQLFNSTWNFNGTWNLTQSNGTEGNDTITLPCRIKQIINMWQEVG
KAMYAPPIRGQIRCSSNITGLILTRDGGNNHNNDTETFRPGGGDMRDNWRSELYKYK
VVKIEPLGVAPTKCKRRVVQSHSGSGGSGSGGHAAVGTIGAMSLGFLGAAGSTMGA
ASITLTVQARLLLSGIVQQQNNLLRAPEPQQHLLQLTVWGIKQLQARVLAVEHYLRD
QQLLGIWGCSGKLICCTAVPWNASWSNKTLDMIWNNMTWMEWEREIDNYTGLIYT
LIEESQNQQEKNEQELLELD
Sequence in bold is the IgE leader sequence; underlined
sequences are the linker sequences; italicized sequences
are repeat 1 optimized for human; dotted underlined
sequences are repeat 2 optimized for human/mouse;
double underlined sequences are repeat 3
optimized for mouse to prevent recombination and large
repeats on the nucleic acid level
Repeat 1 of SEQ ID NO: (above)-optimized for human
VENLWVTVYYGVPVWKEATTTLFCASDAKAYDTEVHNVWATHECVPTDPN
PQEVVLENVTENFNMWKNNMVEQMHEDIIELWDQSLKPCVKLTPLCVTLNCTDLR
NVTNINNSSEGMRGEIKNCSFNITTSIRDKVKKDYALFYRLDVVPIDNDNTSYRLINC
NTSTITQACPKVSFEPIPIHYCTPAGFAILCKDKKFNGTGPCKNVSTVQCTHGIRPVV
STQLLLNGSLAEEEVIIRSSNFTDNAKNIIVQLKESVEINCTRPNNNTVKSIHIGPGRAF
YYTGDIIGDIRQAHCNISRTKWNNTLNQIATKLKEQFGNNKTIVFNQSSGGDPEIVMH
SFNCGGEFFYCNSTQLFNSTWNFNGTWNLTQSNGTEGNDTITLPCRIKQIINMWQEV
GKAMYAPPIRGQIRCSSNITGLILTRDGGNNHNNDTETFRPGGGDMRDNWRSELYKY
KVVKIEPLGVAPTKCKRRVVQSHSGSGGSGSGGHAAVGTIGAMSLGFLGAAGSTMG
AASITLTVQARLLLSGIVQQQNNLLRAPEPQQHLLQLTVWGIKQLQARVLAVEHYLR
DQQLLGIQGCSGKLICCTAVPWNASWSNKTLDMIWNNMTWMEWEREIDNYTGLIY
TLIEESQNQQEKNEQELLELD
Underlined sequences is a linker
Repeat 2 of SEQ ID NO: (above)-optimized for human/mouse
VENLWVTVYYGVPVWKEATTTLFCASDAKAYDTEVHNVWATHECVPTDPN
PQEVVLENVTENFNMWKNNMVEQMHEDIIELWDQSLKPCVKLTPLCVTLNCTDLR
NVTNINNSSEGMRGEIKNCSFNITTSIRDKVKKDYALFYRLDVVPIDNDNTSYRLINC
NTSTITQACPKVSFEPIPIHYCTPAGFAILKCKDKKFNGTGPCKNVSTVQCTHGIRPVV
STQLLLNGSLAEEEVIIRSSNFTDNAKNIIVQLKESVEINCTRPNNNTVKSIHIGPGRAF
YYTGDIIGDIRQAHCNISRTKWNNTLNQIATKLKEQFGNNKTIVFNQSSGGDPEIVMH
SFNCGGEFFYCNSTQLFNSTWNFNGTWNLTQSNGTEGNDTITLPCRIKQIINMWQEV
GKAMYAPPIRGQIRCSSNITGLILTRDGGNNHNNDTETFRPGGGDMRDNWRSELYKY
KVVKIEPLGVAPTKCKRRVVQSHSGSGGSGSGGHAAVGTIGAMSLGFLGAAGSTMG
AASITLTVQARLLLSGIVQQQNNLLRAPEPQQHLLQLTVWGIKQLQARVLAVEHYLR
DQQLLGIWGCSGKLICCTAVPWNASWSNKTLDMIWNNMTWMEWEREIDNYTGLIY
TLIEESQNQQEKNEQELLELD
Underlined sequences is a linker
Repeat 3 of SEQ ID NO: (above)-optimized for mouse
to prevent recombination and large
repeats on the nucleic acid level
VENLWVTVYYGVPVWKEATTTLFCASDAKAYDTEVHNVWATHECVPTDPN
PQEVVLENVTENFNMWKNNMVEQMHEDIIELWDQSLKPCVKLTPLCVTLNCTDLR
NVTNINNSSEGMRGEIKNCSFNITTSIRDKVKKDYALFYRLDVVPIDNDNTSYRLINC
NTSTITQACPKVSFEPIPHYCTPAGFAILKCKDKKFNGTGPCKNVSTVQCTHGIRPVV
STQLLLNGSLAEEEVIIRSSNFTDNAKVIIVQLKESVEINCTRPNNNTVKSIHIGPGRAF
YYTGDIIGDIRQAHCNISRTKWNNTLNQIATKLKEQFGNNKTIVFNQSSGGDPEIVMH
SFNCGGEFFYCNSTQLFNSTWNFNGTWNLTQSNGTEGNDTITLPCRIKQIINMWQEV
GKAMYAPPIRGQIRCSSNITGLILTRDGGNNHNNDTETFRPGGGDMRDNWRSELYKY
KVVKIEPLGVAPTKCKRRVVQSHSGSGGSGSGGHAAVGTIGAMSLGFLGAAGSTMG
AASITLTVQARLLLSGIVQQQNNLLRAPEPQQHLLQLTVWGIKQLQARVLAVEHYLR
DQQLLGIWGCSGKLICCTAVPWNASWSNKTLDMIWNNMTWMEWEREIDNYTGLIY
TLIEESQNQQEKNEQELLELD
Underlined sequences is a linker
AD8_MD64_link14
MDWTWILFLVAAATRVHIVEENLWVTVYYGVPVWKEATTTLFCASDAKAY
DTEVHNVWATHECVPTDPNPQEVVLENVTENFNMWKNNMVEQMHEDIIELWDQSL
KPCVKLTPLCVTLNCTDLRNVTNINNSSEGMRGEIKNCSFNITTSIRDKVKKDYALFY
RLDVVPIDNDNTSYRLINCNTSTITQACPKVSFEPIPIHYCTPAGFAILKCKDKKFNGT
GPCKNVSTVQCTHGIRPVVSTQLLLNGSLAEEEVIIRSSNFTDNAKNIIVQLKESVEIN
CTRPNNNTVKSIHIGPGRAFYYTGDIIGDIRQAHCNISRTKWNNTLNQIATKLKEQFG
NNKTIVFNQSSGGDPEIVMHSFNCGGEFFYCNSTQLFNSTWNFNGTWNLTQSNGTEG
NDTITLPCRIKQIINMWQEVGKAMYAPPIRGQIRCSSNITGLILTRDGGNNHNNDTETF
RPGGGDMRDNWRSELYKYKVVKIEPLGVAPTKCKRRVVQSHSGSGGSGSGGHAAV
GTIGAMSLGFLGAAGSTMGAASITLTVQARLLLSGIVQQQNNLLRAPEPQQHLLQLT
VWGIKQLQARVLAVEHYLRDQQLLGIWGCSGKLICCTAVPWNASWSNKTLDMIWN
NMTWMEWEREIDNYTGLIYTLIEESQNQQEKNEQELLELD
sequence in bold is the IgE leader sequence;
underlined sequence is a linker sequence
VENLWVTVYYGVPVWKEATTTLFCASDAKAYDTEVHNVWATHECVPTDPN
PQEVVLENVTENFNMWKNNMVEQMHEDIIELWDQSLKPCVKLTPLCVTLNCTDLR
NVTNINNSSEGMRGEIKNCSFNITTSIRDKVKKDYALFYRLDVVPIDNDNTSYRLINC
NTSTITQACPKVSFEPIPHYCTPAGFAILKCKDKKFNGTGPCKNVSTVQCTHGIRPVV
STQLLLNGSLAEEEVIIRSSNFTDNAKNIIVQLKESVEINCTRPNNNTVKSIHIGPGRAF
YYTGDIIGDIRQAHCNISRTKWNNTLNQIATKLKEQFGNNKTIVFNQSSGGDPEIVMH
SFNCGGEFFYCNSTQLFNSTWNFNGTWNLTQSNGTEGNDTITLPCRIKQIINMWQEV
GKAMYAPPIRGQIRCSSNITGLILTRDGGNNHNNDTETFRPGGGDMRDNWRSELYKY
KVVKIEPLGVAPTKCKRRVVQ
AVGTIGAMSLGFLGAAGSTMGAASITLTVQARLLLSGIVQQQNNLLRAPEPQ
QHLLQLTVWGIKQLQARVLAVEHYLRDQQLLGIWGCSGKLICCTAVPWNASWSNK
TLDMIWNNMTWMEWEREIDNYTGLIYTLIEESQNQQEKNEQELLELD
001428_MD39_link14_TS1
MDWTWILFLVAAATRVHIVENLWVTVYYGVPVWKEARTTLFCASDAKAY
ETEVHNVWATHACVPTDPNPQEMVLGNVTENFNMWKNDMVDQMHEDVISLWAQ
SLKPCVKLTPLCVTLECTQVNATQGNTTQVNVTQVNGDEMKNCSFNTTTEIRDKKQ
KAYALFYRLDLVPLERENRGDSNSASKYILINCNTSAITQACPKVNFDPIPIHYCTPAG
YAILKCNNKTFNGTGSCNNVSTVQCTHGIKPVVSTQLLLNGSLAEEEIIIRSENLTDNV
KTIIVHLDQSVEIVCTRPNNNTVKSIRIGPGQTFYYTGDIIGNIREAHCNISEKKWHEM
LRRVSEKLAEHFPNKTIKFTSSSGGDLEITTHSFNCRGEFFYCNTSGLFNSTYMPNGTY
MPNGTNNSNSTIILPCRIKQIINMWQEVGRAMYAPPIAGNITCNSNITGLLLVRDGGK
NNNTEIFRPGGGDMRDNWRSELYKYKVVEIKPLGVAPTRCKRRVVGSHSGSGGSGS
GGHAAVGLGAVSLGFLGAAGSTMGAASITLTVQARQLLSGIVQQQSNLLQAPEPQQ
HLLQDTHWGIKQLQTRVLAIEHYLKDQQLLGIWGCSGKLICCTAVPWNSSWSNKSL

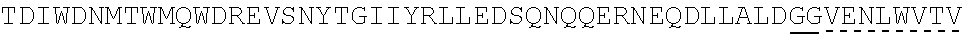

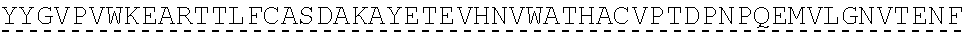

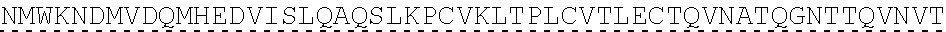

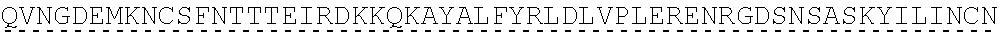

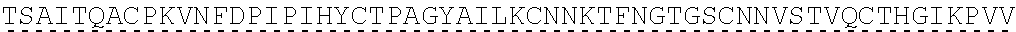

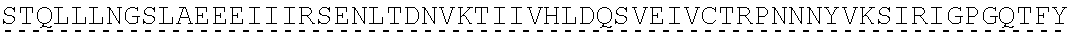

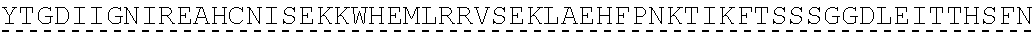

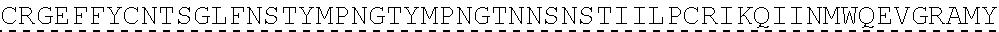

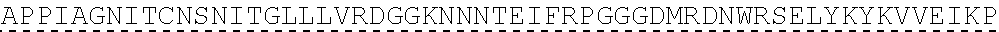

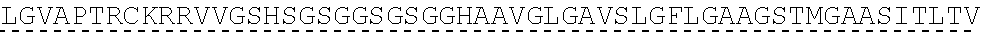

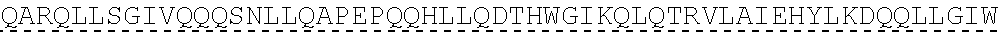

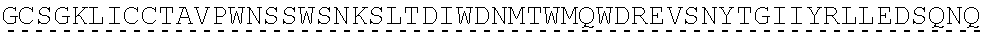

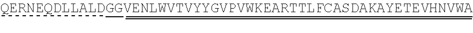

THACVPTDPNPQEMVLGNVTENFNMWKNDMVDQMHEDVISLWAOSLKPCVKLTP
LCVTLECTQVNATQGNTTQVNVTQVNGDEMKNCSFNTTTEIRDKKQKAYALFYRL
DLVPLERENRGDSNSASKYILINCNTSAITQACPKVNFDPIPIHYCTPAGYAILKCNNK
TFNGTGSCNNVSTVQCTHGIKPVVSTQLLLNGSLAEEEIIIRSENLTDNVKTIIVHLDQS
VEIVCTRPNNNTVKSIRIGPGQTFYYTGDIIGNIREAHCNISEKKWHEMLRRVSEKLAE
HFPNKTIKFTSSSGGDLEITTHSFNCRGEFFYCNTSGLFNSTYMPNGTYMPNGTNNSN
STIILPCRIKQIINMWQEVGRAMYAPPIAGNITCNSNITGLLLVRDGGKNNNTEIFRPG
GGDMRDNWRSELYKYKVVEIKPLGVAPTRCKRRVVGSHSGSGGSGSGGHAAVGLG
AVSLGFLGAAGSTMGAASITLTVQARQLLSGIVQQQSNLLQAPEPQQHLLQDTHWGI
KQLQTRVLAIEHYLKDQQLLGIWGCSGKLICCTAVPWNSSWSNKSLTDIWDNMTW
MQWDREVSNYTGIIYRLLEDSQNQQERNEQDLLALD
Sequence in bold is the IgE leader sequence;
underlined sequences are the linker
sequences; italicized sequences are repeat 1
optimized for human; dotted underlined
sequences are repeat 2 optimized for human/
mouse; double underlined sequences are repeat 3
optimized for mouse to prevent recombination and
large repeats on the nucleic acid level
Repeat 1 of SEQ ID NO: (above)-optimized for human
VENLWVTVYYGVPVWKEARTTLFCASDAKAYETEVHNVWATHACVPTDPN
PQEMVLGNVTENFNMWKNDMVDQMHEDVISLWAQSLKPCVKLTPLCVTLECTQV
NATQGNTTQVNVTQVNGDEMKNCSFNTTTEIRDKKQKAYALFYRLDLVPLERENRG
DSNSASKYILINCNTSAITQACPKVNFDPIPIHYCTPAGYAILKCNNKTFNGTGSCNNV
STVQCTHGIKPVVSTQLLLNGSLAEEEIIIRSENLTDNVKTIIVHLDQSVEIVCTRPNNN
TVKSIRIGPGQTFYYTGDIIGNIREAHCNISEKKWHEMLRRVSEKLAEHFPNKTIKFTS
SSGGDLEITTHSFNCRGEFFYCNTSGLFNSTYMPNGTYMPNGTNNSNSTIILPCRIKQII
NMWQEVGRAMYAPPIAGNITCNSNITGLLLVRDGGKNNNTEIFRPGGGMRDNWRS
ELYKYKVVEIKPLGVAPTRCKRRVVGSHSGSGGSGSGGHAAVGLGAVSLGFLGAAG
STMGAASITLTVQARQLLSGIVQQQSNLLQAPEPQQHLLQDTHWGIKQLQTRVLAIE
HYLKDQQLLGIWGCSGKLICCTAVPWNSSWSNKSLTDIWDNMTWMQWDREVSNY
TGIIYRLLEDSQNQQERNEQDLLALD
Repeat 2 of SEQ ID NO: (above)-optimized for human/mouse
VENLWVTVYYGVPVWKEARTTLFCASDAKAYETEVHNWATHACVPTDPN
PQEMVLGNVTENFNMWKNDMVDQMHEDVISLWAQSLKPCVKLTPLCVTLECTQV
NATQGNTTQVNVTQVNGDEMKNCSFNTTTEIRDKKQDAYALFYRLDLVPLERENRG
DSNSASKYILINCNTSAITQACPKVNFDPIPIHYCTPAGYAILKCNNKTFNGTGSCNNV
STVQCTHGIKPVVSTQLLLNGSLAEEEIIIRSENLTDNVKTIIVHLDQSVEIVCTRPNNN
TVKSIRIGPGQTFYYTGDIIGNIREAHCNISEKKWHEMLRRVSEKLAEHFPNKTIKFTS
SSGGDLEITTHSFNCRGEFFYCNTSGLFNSTYMPNGTYMPNGTNNSNSTIILPCRIKQII
NMWQEVGRAMYAPPIAGNITCNSNITGLLLVRDGGKNNNTEIFRPGGGDMRDNWRS
ELKYKVVEIKPLGVAPTRCKRRVVGSHSGSGGSGSGGHAAVGLGAVSLGFLGAAG
STMGAASITLTVQARQLLSGIVQQQSNLLQAPEPQQHLLQDTHWGIKQLQTRVLAIE
HYLKDQQLLGIWGCSGKLICCTAVPWNSSWSNKSLTDIWDNMTWMQWDREVSNY
TGIIYRLLEDSQNQQERNEQDLLALD
Repeat 3 of SEQ ID NO: (above)-optimized for mouse
to prevent recombination and large repeats on
the nucleic acid level during DNA replication.
VENLWVTVYYGVPVWKEARTTLFCASDAKAYETEVHNVWATHACVPTDPN
PQEMVLGNVTENFNMWKNDMVDQMHEDVISLWAQSLKPCVKLTPLCVTLECTQV
NATQGNTTQVNVTQVNGDEMKNCSFNTTTEIRDKKQKAYALFYRLDLVPLERENRG
DSNSASKYILINCNTSAITQACPKVNFDPIPIHYCTPAGYAILKCNNKTGNGTGSCNNV
STVQCTHGIKPVVSTQLLLNGSLAEEEIIIRSENLTDNVKTIIVHLDQSVEIVCTRPNNN
TVKSIRIGPGQTFYYTGDIIGNIREAHCNISEKKWHEMLRRVSEKLAEHFPNKTIKFTS
SSGGDLEITTHSFNCRGEFFYCNTSGLFNSTYMPNGTYMPNGTNNSNSTIILPCRIKQII
NMWQEVGRAMYAPPIAGNITCNSNITGLLLVRDGGKNNNTEIFRPGGGDMRDNWRS
ELYKYKVVEIKPLGVAPTRCKRRVVGSHSGSGGSGSGGHAAVGLGAVSLGFLGAAG
STMGAASITLTVQARQLLSGIVQQQSNLLQAPEPQQHLLQDTHWGIKQLQTRVLAIE
HYLKDQQLLGIWGCSGKLICCTAVPWNSSWSNKSLTDIWDNMTWMQWDREVSNY
TGIIYRLLEDSQNQQERNEQDLLALD
001428_MD39_link14_pVax
MDWTWILFLVAAATRVHIVENLWVTVYYGVPVWKEARTTLFCASDAKAY
ETEVHNVWATHACVPTDPNPQEMVLGNVTENFNMWKNDMVSQMHEDVISLWAQ
SLKPCVKLTPLCVTLECTQVNATQGNTTQVNVTQVNGDEMKNCSFNTTTEIRDKKQ
KAYALFYRLDLVPLERENRGDSNSASKYILINCNTSAITQACPKVNFDPIPIHYCTPAG
YAILKCNNKTFNGTGSCNNVSTVQCTHGIKPVVSTQLLLNGLSAEEEIIIRSENLTDNV
KTIIVHLDQSVEIVCTRPNNNTVKSIRIGPGQTFYYTGDIIGNIREAHCNISEKKWHEM
LRRVSEKLAEHFPNKTIKFTSSSGGDLEITTHSFNCRGEFFYCNTSGLFNSTYMPNGTY
MPNGTNNSNSTIILPCRIKQIINMWQEVGRAMYAPPIAGNITCNSNITGLLLVRDGGK
NNNTEIFRPGGGDMRDNWRSELYKYKVVEIKPLGVAPTRCKRRVVGSHSGSGGSGS
GGHAAVGLGAVSLGFLGAAGSTMGAASITLTVQARQLLSGIVQQQSNLLQAPEPQQ
HLLQDTHWGIKQLQTRVLAIEHYLKDQQLLGIWGCSGKLICCTAVPWNSSWSNKSL
TDIWDNMTWMQWDREVSNYTGIIYRLLEDSQNQQERNEQDLLALD
Sequence in bold is an IgE leader sequence; underlined
sequence is a linker sequence
VENLWVTVYYGVPVWKEARTTLFCASDAKAYETEVHNVWATHACVPTDPN
PQEMVLGNVTENFNMWKNDMVDQMHEDVISLWAQSLKPCVKLTPLCVTLECTQV
NATQGNTTQVNVTQVNGDEMKNCSFNTTTEIRDKKQKAYALFYRLDLVPLERENRG
DSNSASKYILINCNTSAITQACPKVNFDPIPHYCTPAGYAILKCNNKTFNGTGSCNNV
STVQCTHGIKPVVSTLLLNGSLAEEEIIIRSENLTDNVKTIIVHLDQSVEIVCTRPNNN
TVKSIRIGPGQTFYYTGDIIGNIREAHCNISEKKWHEMLRRVSEKLAEHFPNKTIKFTS
SSGGDLEITTHSFNCRGEFFYCNTSGLFNSTYMPNGTYMPNGTNNSNSTIILPCRIKQII
NMWQEVGRAMYAPPIAGNITCNSNITGLLLVRDGGKNNNTEIFRPGGGDMRDNWRS
ELYKYKVVEIKPLGVAPTRCKRRVV
AVGLGAVSLGFLGAAGSTMGAASITLTVQARQLLSGIVQQQSNLLQAPEPQQ
HLLQDTHWGIKQLQTRVLAIEHYLKDQQLLGIWGCSGKLICCTAVPWNSSWSNKSL
TDIWDNMTWMQWDREVSNYTGIIYRLLEDSQNQQERNEQDLLALD
In some embodiments, the expressible nucleic acid sequence
comprises a nucleic acid sequence encoding a trimer
peptide, wherein the nuceic acid sequence comprises
any one or more of the following sequences or a sequence
that comprises at least about 70%, 75%, 80%,
85%, 90%, 95%, 96%, 97%, 98%, 99% or 100% sequence
identity to the following sequences:
BG505_SOSIP_MD39-nucleic acid
ATGGACTGGACATGGATTCTGTTCCTGGTCGCTGCCGCTACAAGAGTGCAT
TCCGCCGAAAACCTGTGGGTCACCGTCTCTATGGAGTGCCCGTGTGGAAGGAC
GCCGAGACTACGCTGTTCTGCGCCAGCGATGCCAAGGCCTACGAGACAGAGAAG
CACAACGTGTGGGCAACCCACGCATGCGTGCCTACAGACCCAAACCCCAGGAG
ATCCACCTGGAGAATGTGACAGAGGAGTTTAACATGTGGAAGAACAATATGGTG
GAGCAGATGCACGAGGACATCATCTCCCTGTGGGATCAGTCTCTGAAGCCCTGCG
TGAAGCTGACCCCTCTGTGCGTGACACTGCAGTGTACCAACGTGACAAACAATAT
CACCGACGATATGCGGGGCGAGCTGAAGAATTGTAGCTTCAACATGACCACAGA
GCTGAGGGACAAGAAGCAGAAGGTGTACTCCCTGTTTTATAGACTGGATGTGGT
GCAGATCAATGAGAACCAGGGCAATCGGTCTAACAATAGCAACAAGGAGTACCG
CCTGATCAATTGCAACACCTCCGCCATCACACAjGGCCTGTCCTAAGGTGTCTTTC
GAGCCTATCCCAATCCACTATTGCGCCCCAGCCGGCTTCGCCATCCTGAAGTGTA
AGGATAAGAAGTTTAACGGAACCGGACCATGCCCTTCCGTGTCTACCGTGCAGTG
TACACACGGCATCAAGCCTGTGGTGTCTACACAGCTGCTGCTGAATGGCAGCCTG
GCCGAGGAGGAAGTGATCATCAGGTCTGAGAACATCACCAACAATGCCAAGAAT
ATCCTGGTGCAGCTGAACACACCAGTGCAGATCAATTGCACCCGGCCCAACAAT
AACACAGTGAAGTCTATCCGCATCGGCCCAGGCCAGGCCTTTTACTATACCGGCG
ACATCATCGGCGATATCAGACAGGCCCACTGTAATGTGAGCAAGGCCACCTGGA
ACGAGACACTGGGCAAGGTGGTGAAGCAGCTGAGGAAGCACTTCGGCAATAACA
CCATCATCAGATTTGCACAGAGCTCCGGCGGCGACCTGGAGGTGACCACACACT
CCTTCATTGCGGCGGCGAGTTCTTTTACTGTAACACAAGCGGCCTGTTTAATTCC
ACCTGGATCTCCAACACATCTGTGCAGGGCAGCAATTCCACCGGCAGCAACGATT
CCATCACACTGCCATGCCGGATCAAGCAGATCATCAACATGTGGCAGCGCATCG
GCCAGGCCATGTATGCCCCTCCCATCCAGGGCGTGATCAGATGCGTGAGCAATAT
CACCGGCCTGATCCTGACACGCGACGGCGGCTCTACCAACAGCACCACAGAGAC
ATTCCGGCCCGGCGGCGGCGACATGAGGGATAACTGGAGATCTGAGCTGTACAA
GTATAAGGTGGTGAAGATCGAGCCTCTGGGAGTGGCACCAACCAGGTGCAAGAG
GAGAGTGGTGGGCCGGCGCAGGAGACGGCGCGCAGTGGGCATCGGGCCGTGTC
CCTGGGCTTTCTGGGAGCAGCAGGCTCCACAATGGGAGCAGCCTCTATGACCCTG
ACAGTGCAGGCCAGGAATCTGCTGAGCGGCATCGTGCAGCAGCAGTCCAACCTG
CTGAGAGCCCCAGAGCCCCAGCAGCACCTGCTGAAGGACACCCACTGGGGCATC
AAGCAGCTGCAGGCCAGGGTGCTGGCAGTGGAGCACTATCTGAGAGATCAGCAG
CTGCTGGGCATCTGGGGCTGTAGCGGCAAGCTGATCTGCTGTACCAATGTGCCCT
GGAACTCTAGCTGGTCTAATCGCAACCTGAGCGAGATCTGGGACAATATGACCT
GGCTGCAGTGGGATAAGGAGATCTCCAACTACACACAGATCATCTATGGCCTGCT
GGAAGAATCTCAGAATCAGCAGGAAAAGAATGACCAGGATCTGCTGGCACTGGA
TTGATAACTCGAG
BG505_MD39_GRSF (Glycan)-nucleic acid
ATGGACTGGACATGGATTCTGTTCCTGGTCGCTGCCGCTACAAGAGTGCAT
TCCGCCGAAAACCTGTGGGTCACCGTCTACTATGGAGTGCCCGTGTGGAAGGAC
GCCGAGACTACGCTGTTCTGCGCCAGCGATGCCAAGGCCTACGAGACAGAGAAG
CACAACGTGTGGGCAACCCACGCATGCGTGCCTACAGACCCAAACCCCCAGGAG
ATCCACCTGGAGAATGTGACAGAGGAGTTTAACATGTGGAAGAACAATATGGTG
GAGCAGATGCACGAGGACATCATCTCCCTGTGGGATCAGTCTCTGAAGCCCTGCG
TGAAGCTGACCCCTCTGTGCGTGACACTGCAGTGTACCAACGTGACAAACAATAT
CACCGACGATATGCGGGGCGAGCTGAAGAATTGTAGCTTCAACATGACCACAGA
GCTGAGGGACAAGAAGCAGAAGGTGTACTCCCTGTTTTATAGACTGGATGTGGT
GCAGATCAATGAGAACCAGGGCAATCGGTCTAACAATAGCAACAAGGAGTACCG
CCTGATCAATTGCAACACCTCCGCCATCACACAGGCCTGTCCTAAGGTGTCTTTC
GAGCCTATCCCAATCCACTATTGCGCCCCAGCCGGCTTCGCCATCCTGAAGTGTA
AGGATAAGAAGTTTAACGGAACCGGACCATGCCCTTCCGTGTCTACCGTGCAGTG
TACACACGGCATCAAGCCTGTGGTGTCTACACAGCTGCTGCTGAATGGCAGCCTG
GCCGAGGAGGAAGTGATCATCAGGTCTGAGAACATCACCAACAATGCCAAGAAT
ATCCTGGTGCAGCTGAACACACCAGTGCAGATCAATTGCACCCGGCCCAACAAT
AACACAGTGAAGTCTATCCGCATCGGCCCAGGCCAGGCCTTTTACTATACCGGCG
ACATCATCGGCGATATCAGACAGGCCCACTGTAATGTGAGCAAGGCCACCTGGA
ACGAGACACTGGGCAAGGTGGTGAAGCAGCTGAGGAAGCACTTCGGCAATAACA
CCATCATCAGATTTGCACAGAGCTCCGGCGGCGACCTGGAGGTGACCACACACT
CCTTCAATTGCGGCGGCGAGTTCTTTTACTGTAACACAAGCGGCCTGTTTAATTCC
ACCTGGATCTCCAACACATCTGTGCAGGGCAGCAATTCCACCGGCAGCAACGATT
CCATCACACTGCCATGCCGGATCAAGCAGATCATCAACATGTGGCAGCGCATCG
GCCAGGCCATGTATGCCCCTCCCATCCAGGGCGTGATCAGATGCGTGAGCAATAT
CACCGGCCTGATCCTGACACGCGACGGCGGCTCTACCAACAGCACCACAGAGAC
ATTCCGGCCCGGCGGCGGCGACATGAGGGATAACTGGAGATCTGAGCTGTACAA
GTATAAGGTGGTGAAGATCGAGCCTCTGGGAGTGGCACCAACCAGGTGCAAGAG
GAGAGTGGTGGGCCGGCGCAGGAGACGGCGCGCAGTGGGCATCGGAGCCGTGTC
CCTGGGCTTTCTGGGAGCAGCAGGCTCCACAATGGGAGCAGCCTCTATGACCCTG
ACAGTGCAGGCCAGGAATCTGCTGAGCGGCATCGTGCAGCAGCAGTCCAACCTG
CTGAGAGCCCCAGAGCCCCAGCAGCACCTGCTGAAGGACACCCACTGGGGCATC
AAGCAGCTGCAGGCCAGGGTGCTGGCAGTGGAGCACTATCTGAGAGATCAGCAG
CTGCTGGGCATCTGGGGCTGTAGCGGCAAGCTGATCTGCTGTACCAATGTGCCCT
GGAACTCTAGCTGGTCTAATCGCAACCTGAGCGAGATCTGGGACAATATGACCT
GGCTGAACTGGAGCAAGGAGATCTCCAACTACACACAGATCATCTATGGCCTGC
TGGAAGAATCTCAGAATCAGCAGGAAAAGAATAACCAGAGCCTGCTGGCACTGG
ATTGATAA
B505_SOSIP_MD39_CPG9.2-nucleic acid
ATGGATTGGACTTGGATTCTGTTCCTGGTCGCAGCAGCCACACGAGTGCAT
AGCGGGGGAAATAGTAGCGGCAGCCTGGGGTTCCTGGGAGCAGCAGGCTCCACC
ATGGGAGCAGCATCTATGACCCTGACAGTGCAGGCCAGGAATCTGCTGTCTGGC
ATCGTGCAGCAGCAGAGCAACCTGCTGAGAGCCCCAGAGCCCCAGCAGCACCTG
CTGAAGGACACCCACTGGGGCATCAAGCAGCTGCAGGCCCGGGTGCTGGCAGTG
GAGCACTACCTGCGCGATCAGCAGCTGCTGGGAATCTGGGGATGCAGCGGCAAG
CTGATCTGCTGTACAAATGTGCCTTGGAACAGCTCCTGGTCCAATAGGAACCTGT
CTGAGATCTGGGACAATATGACCTGGCTGAACTGGTCTAAGGAGATCAGCAATT
ACACACAGATCATCTATGGCCTGCTGGAGGAGAGCCAGAATCAGAACGAGTCCA
ATGAGCAGGATCTGGGCGGCAACGGCAGCGGCGGCGGCAGCGGCTCCGGCGGC
AACGGCTCTAGCGGCCTGTGGGTGACCGTGTACTATGGCGTGCCCGTGTGGAAG
GACGCCGAGACTACGCTGTTCTGCGCCTCCGATGCCAAGGCCTATGAGACAGAG
AAGCACAACGTGTGGGCAACCCACGCATGCGTGCCAACAGACCCTAACCCACAG
GAGATCCACCTGGAGAATGTGACCGAGGAGTTTAACATGTGGAAGAACAATATG
GTGGAGCAGATGCACGAGGACATCATCAGCCTGTGGGATCAGTCCCTGAAGCCT
TGCGTGAAGCTGACCCCACTGTGCGTGACACTGCAGTGTACCAACGTGACAAAC
AATATCACCGACGATATGAGGGGCGAGCTGAAGAATTGTTCTTTCAACATGACC
ACAGAGCTGAGGGACAAGAAGCAGAAAGTGTACAGCCTGTTTTATAGACTGGAT
GTGGTGCAGATCAATGAGAACCAGGGCAATAGGAGCAACAATTCCAACAAGGA
GTACAGACTGATCAATTGCAACACCAGCGCCATCACACAGGCCTGTCCAAAGGT
GTCCTTCGAGCCCATCCCTATCCACTATTGCGCACCAGCAGGATTCGCAATCCTG
AAGTGTAAGGATAAGAAGTTTAACGGAACCGGACCATGCCCATCTGTGAGCACC
GTGCAGTGTACACACGGCATCAAGCCAGTGGTGTCCACACAGCTGCTGCTGAAT
GGCTCTCTGGCCGAGGAGGAAGTGATCATCCGGAGCGAGAACATCACCAACAAT
GCCAAGAATATCCTGGTGCAGCTGAACACACCCGTGCAGATCAATTGCACCCGG
CCTAACAATAACACAGTGAAGTCCATCAGGATCGGACCAGGACAGGCCTTTTAC
TATACCGGCGACATCATCGGCGATATCCGCCAGGCCCACTGTAACGTGAGCAAG
GCCACCTGGAACGAGACACTGGGCAAGGTGGTGAAGCAGCTGAGGAAGCACTTC
GGCAATAACACCATCATCAGATTTGCACAGTCCTCTGGCGGCGACCTGGAGGTG
ACCACACACTCCTTCAACTGCGGCGGCGAGTTCTTTTACTGTAACACATCTGGCC
TGTTTAATAGCACCTGGATCTCTAACACAAGCGTGCAGGGCTCCAATTCTACCGG
CTCCAACGATTCTATCACACTGCCCTGCCGGATCAAGCAGATCATCAACATGTGG
CAGAGGATCGGACAGGCAATGTACGCCCCTCCCATCCAGGGCGTGATCAGATGC
GTGAGCAATATCACCGGCCTGATCCTGACACGCGACGGCGGCAGCACCAACTCC
ACCACAGAGACATTCAGACCCGGCGGCGGCGACATGAGGGATAACTGGAGATCC
GAGCTGTATAAGTATAAAGTCGTGAAGATTGAGCCACTGGGCGTCGCACCAACA
AGATGTAATAGAAGCTGATAA
BG505_SOSIP_MD39_link14-nucleic acid
ATGGACTGGACATGGATTCTGTTCCTGGTCGCTGCCGCTACAAGAGTGCAT
TCCGCCGAAAACCTGTGGGTCACCGTCTACTATGGAGTGCCCGTGTGGAAGGAC
GCCGAGACTACGCTGTTCTGCGCCAGCGATGCCAAGGCCTACGAGACAGAGAAG
CACAACGTGTGGGCAACCCACGCATGCGTGCCTACAGACCCAAACCCCAGGAG
ATCCACCTGGAGAATGTGACAGAGGAGTTTAACATGTGGAAGAACAATATGGTG
GAGCAGATGCACGAGGACATCATCTCCCTGTGGGATCAGTCTCTGAAGCCCTGCG
TGAAGCTGACCCCTCTGTGCGTGACACTGCAGTGTACCAACGTGACAAACAATAT
CACCGACGATATGCGGGGCGAGCTGAAGAATTGTAGCTTCAACATGACCACAGA
GCTGAGGGACAAGAAGCAGAAGGTGTACTCCCTGTTTTATAGACTGGATGTGGT
GCAGATCAATGAGAACCAGGGCAATCGGTCTAACAATAGCAACAAGGAGTACCG
CCTGATCAATTGCAACACCTCCGCCATCACACAGGCCTGTCCTAAGGTGTCTTTC
GAGCCTATCCCAATCCACTATTGCGCCCCAGCCGGCTTCGCCATCCTGAAGTGTA
AGGATAAGAAGTTTAACGGAACCGGACCATGCCCTTCCGTGTCTACCGTGCAGTG
TACACACGGCATCAAGCCTGTGGTGTCTACACAGCTGCTGCTGAATGGCAGCCTG
GCCGAGGAGGAAGTGATCATCAGGTCTGAGAACATCACCAACAATGCCAAGAAT
ATCCTGGTGCAGCTGAACACACCAGTGCAGATCAATTGCACCCGGCCCAACAAT
AACACAGTGAAGTCTATCCGCATCGGCCCAGGCCAGGCCTTTTACTATACCGGCG
ACATCATCGGCGATATCAGACAGGCCCACTGTAATGTGAGCAAGGCCACCTGGA
ACGAGACACTGGGCAAGGTGGTGAAGCAGCTGAGGAAGCACTTCGGCAATAACA
CCATCATCAGATTTGCACAGAGCTCCGGCGGCGACCTGGAGGTGACCACACACT
CCTTCAATTGCGGCGGCGAGTTCTTTTACTGTAACACAAGCGGCCTGTTTAATTCC
ACCTGGATCTCCAACACATCTGTGCAGGGCAGCAATTCCACCGGCAGCAACGATT
CCATCACACTGCCATGCCGGATCAAGCAGATCATCAACATGTGGCAGCGCATCG
GCCAGGCCATGTATGCCCCTCCCATCCAGGGCGTGATCAGATGCGTGAGCAATAT
CACCGGCCTGATCCTGACACGCGACGGCGGCTCTACCAACAGCACCACAGAGAC
ATTCCGGCCCGGCGGCGGCGACATGAGGGATAACTGGAGATCTGAGCTGTACAA
GTATAAGGTGGTGAAGATCGAGCCTCTGGGAGTGGCACCAACCAGGTGCAAGAG
GAGAGTGGTGGGCTCTCACAGCGGCTCCGGCGGCTCTGGCAGCGGCGGCCACGC
CGCAGTGGGCATCGGAGCCGTGTCCCTGGGCTTTCTGGGAGCAGCAGGCTCCAC
AATGGGAGCAGCCTCTATGACCCTGACAGTGCAGGCCAGGAATCTGCTGAGCGG
CATCGTGCAGCAGCAGTCCAACCTGCTGAGAGCCCCAGAGCCCCAGCAGCACCT
GCTGAAGGACACCCACTGGGGCATCAAGCAGCTGCAGGCCAGGGTGCTGGCAGT
GGAGCACTATCTGAGAGATCAGCAGCTGCTGGGCATCTGGGGCTGTAGCGGCAA
GCTGATCTGCTGTACCAATGTGCCCTGGAACTCTAGCTGGTCTAATCGCAACCTG
AGCGAGATCTGGGACAATATGACCTGGCTGCAGTGGGATAAGGAGATCTCCAAC
TACACACAGATCATCTATGGCCTGCTGGAAGAATCTCAGAATCAGCAGGAAAAG
AATGAACAGGATCTGCTGGCACTGGATTGATAA
BG505_SOSIP_MD39_trimer string 1 (TS1)-nucleic acid
ATGGACTGGACATGGATTCTGTTCCTGGTCGCTGCCGCTACAAGAGTGCAT
TCCGCCGAAAACCTGTGGGTCACCGTCTACTATGGAGTGCCCGTGTGGAAGGAC
GCCGAGACTACGCTGTTCTGCGCCAGCGATGCCAAGGCCTACGAGACAGAGAAG
CACAACGTGTGGGCAACCCACGCATGCGTGCCTACAGACCCAAACCCCCAGGAG
ATCCACCTGGAGAATGTGACAGAGGAGTTTAACATGTGGAAGAACAATATGGTG
GAGCAGATGCACGAGGACATCATCTCCCTGTGGGATCAGTCTCTGAAGCCCTGCG
TGAAGCTGACCCCTCTGTGCGTGACACTGCAGTGTACCAACGTGACAAACAATAT
CACCGACGATATGCGGGGCGAGCTGAAGAATTGTAGCTTCAACATGACCACAGA
GCTGAGGGACAAGAAGCAGAAGGTGTACTCCCTGTTTTATAGACTGGATGTGGT
GCAGATCAATGAGAACCAGGGCAATCGGTCTAACAATAGCAACAAGGAGTACCG
CCTGATCAATTGCAACACCTCCGCCATCACACAGGCCTGTCCTAAGGTGTCTTTC
GAGCCTATCCCAATCCACTATTGCGCCCCAGCCGGCTTCGCCATCCTGAAGTGTA
AGGATAAGAAGTTTAACGGAACCGGACCATGCCCTTCCGTGTCTACCGTGCAGTG
TACACACGGCATCAAGCCTGTGGTGTCTACACAGCTGCTGCTGAATGGCAGCCTG
GCCGAGGAGGAAGTGATCATCAGGTCTGAGAACATCACCAACAATGCCAAGAAT
ATCCTGGTGCAGCTGAACACACCAGTGCAGATCAATTGCACCCGGCCCAACAAT
AACACAGTGAAGTCTATCCGCATCGGCCCAGGCCAGGCCTTTTACTATACCGGCG
ACATCATCGGCGATATCAGACAGGCCCACTGTAATGTGAGCAAGGCCACCTGGA
ACGAGACACTGGGCAAGGTGGTGAAGCAGCTGAGGAAGCACTTCGGCAATAACA
CCATCATCAGATTTGCACAGAGCTCCGGCGGCGACCTGGAGGTGACCACACACT
CCTTCAATTGCGGCGGCGAGTTCTTTTACTGTAACACAAGCGGCCTGTTTAATTCC
ACCTGGATCTCCAACACATCTGTGCAGGGCAGCAATTCCACCGGCAGCAACGATT
CCATCACACTGCCATGCCGGATCAAGCAGATCATCAACATGTGGCAGCGCATCG
GCCAGGCCATGTATGCCCCTCCCATCCAGGGCGTGATCAGATGCGTGAGCAATAT
CACCGGCCTGATCCTGACACGCGACGGCGGCTCTACCAACAGCACCACAGAGAC
ATTCCGGCCCGGCGGCGGCGACATGAGGGATAACTGGAGATCTGAGCTGTACAA
GTATAAGGTGGTGAAGATCGAGCCTCTGGGAGTGGCACCAACCAGGTGCAAGAG
GAGAGTGGTGGGCTCTCACAGCGGCTCCGGCGGCTCTGGCAGCGGCGGCCACGC
CGCAGTGGGCATCGGAGCCGTGTCCCTGGGCTTTCTGGGAGCAGCAGGCTCCAC
AATGGGAGCAGCCTCTATGACCCTGACAGTGCAGGCCAGGAATCTGCTGAGCGG
CATCGTGCAGCAGCAGTCCAACCTGCTGAGAGCCCCAGAGCCCCAGCAGCACCT
GCTGAAGGACACCCACTGGGGCATCAAGCAGCTGCAGGCCAGGGTGCTGGCAGT
GGAGCACTATCTGAGAGATCAGCAGCTGCTGGGCATCTGGGGCTGTAGCGGCAA
GCTGATCTGCTGTACCAATGTGCCCTGGAACTCTAGCTGGTCTAATCGCAACCTG
AGCGAGATCTGGGACAATATGACCTGGCTGCAGTGGGATAAGGAGATCTCCAAC
TACACACAGATCATCTATGGCCTGCTGGAAGAATCTCAGAATCAGCAGGAAAAG
AATGAACAGGATCTGCTGGCACTGGATGGCGGCGCCGAAAACCTGTGGGTCACC
GTGTACTACGGAGTCCCCGTGTGGAAAGATGCAGAGACAACCCTGTTCTGCGCTT
CCGACGCTAAAGCTTACGAGACAGAAAAACACAACGTGTGGGCCACTCATGCCT
GCGTGCCTACAGACCCTAACCCACAGGAAATCCACCTGGAGAATGTGACGGAGG
AGTTTAACATGTGGAAGAATAACATGGTCGAGCAGATGCATGAAGATATCATTT
CCTTATGGGACCAATCCCTGAAGCCTTGCGTGAAGCTGACCCCACTGTGCGTGAC
ACTGCAATGCACTAACGTGACCAATAACATTACCGACGATATGCGCGGCGAAGCT
GAAGAACTGCTCTTTCAACATGACTACCGAGCTGAGAGATAAGAAACAGAAAGT
GTACAGCCTGTTTTATCGGTTAGATGTGGTGCAGATCAATGAAAACCAGGGCAAT
CGGTCCAACAATTCTAACAAGGAATATCGCCTGATCAATTGTAACCCTCCGCCA
TTACCCAGGCTTGCCCTAAGGTGTCTTTCGAGCCCATCCCTATCCACTATTGCGCC
CCAGCTGGATTTGCTATCCTGAAGTGTAAGGACAAAAAGTTTAACGGGACCGGA
CCATGTCCTAGCGTGTCCACTGTGCAGTGCACCCATGGCATCAAGCCTGTGGTGT
CCACCCAACTTCTGCTGAATGGCTCTCTGGCTGAAGAAGAAGTGATCATTAGGTC
CGAAAATATTACTAATAACGCTAAAAATATCCTGGTCCAGCTGAACACGCCTGTC
CAGATCAATTGTACCCGGCCAAATAACAACACAGTGAAGTCTATCAGAATCGGC
CCAGGCCAGGCCTTCTACTACACAGGCGACATTATCGGCGATATTCGCCAGGCCC
ACTGTAATGTGAGCAAAGCTACATGGAATGAGACACTGGGCAAGGTAGTCAAAC
AGCTGAGAAAACATTTTGGAAACAACACCATCATCCGCTTTGCACAGTCTAGCGG
CGGCGACCTGGAGGTAACTACCCACAGCTTCAATTGTGGCGGCGAGTTCTTTTAC
TGTAATACCAGCGGCCTGTTTAATAGTACTTGGATCAGCAACACATCTGTGCAGG
GCTCTAACTCCACTGGCTCTAACGATAGCATCACACTGCCTTGTCGGATCAAGCA
AATCATCAACATGTGGCAAAGGATTGGGCAGGCTATGTATGCCCCTCCAATCCAG
GGCGTGATCCGGTGCGTGAGCAACATTACAGGCCTGATCCTGACAAGAGACGGC
GGCTCCACCAACTCTACTACCGAGACATTCCGGCCCGGCGGCGGCGACATGCGT
GATAACTGGCGCAGCGAACTGTATAAATATAAAGTGGTGAAGATCGAGCCTCTG
GGCGTGGCCCCAACTAGGTGTAAAAGAAGGGTCGTCGGCTCCCACAGCGGCAGC
GGCGGCTCCGGCTCTGGCGGCCACGCGGCTGTCGGCATCGGCGCCGTGAGCCTG
GGCTTTCTGGGCGCCGCCGGCTCCACTATGGGCGCAGCCTCTATGACCCTGACTG
TCCAGGCTAGAAATCTGCTGTCTGGAATCGTGCAGCAGCAGTCTAACCTGCTGAG
GGCACCTGAGCCACAACAGCACCTGCTGAAGGATACACATTGGGGCATCAAGCA
GTTACAAGCCAGGGTGCTGGCCGTGGAACACTACCTGCGCGATCAGCAATTACT
GGGCATTTGGGGATGCTCTGGCAAGCTGATTTGTTGCACCAATGTGCCCTGGAAC
TCCTCTTGGAGCAACAGAAACCTGTCCGAAATCTGGGATAACATGACATGGCTGC
AGTGGGACAAGGAAATTTCCAATTATACCCAGATCATCTATGGACTGCTGGAAG
AAAGTCAGAATCAGCAGGAGAAGAATGAACAGGATCTGCTGGCACTGGATGGCG
GCGCCGAAAACCTGTGGGTCACCGTGTATTATGGAGTGCCAGTGTGGAAGGACG
CCGAGACCACACTGTTTTGTGCCTCTGATGCCAAGGCCTACGAGACCGAGAAGC
ACAACGTGTGGGCCACCCACGCCTGCGTGCCCACAGACCCAAATCCTCAGGAGA
TCCACCTGGAGAACGTGACCGAGGAGTTTAACATGTGGAAGAACAATATGGTGG
AGCAGATGCACGAGGATATCATCTCTCTGTGGGATCAGTCTCTGAAGCCATGTGT
GAAGCTGACCCCACTGTGCGTGACCCTGCAGTGTACAAATGTGACAAACAACAT
CACAGATGACATGAGAGGCGAGCTGAAGAACTGTTCCTTCAATATGACCACCGA
GCTGAGAGACAAGAAGCAGAAGGTGTATTCTCTGTTTTACCGGCTGGACGTGGT
GCAGATCAACGAGAATCAAGGGCAATCGGTCTAACAACTCCAATAAGGAGTATAG
ACTGATCAACTGCAACACCTCTGCCATCACCCAGGCCTGTCCTAAGGTGTCCTTT
GAGCCAATCCCAATCCACTATTGCGCCCCTGCCGGCTTTGCCATCCTGAAGTGCA
AGGACAAGAAGTTTAACGGCACAGGCCCCTGCCCATCCGTGAGCACAGTGCAGT
GTACCCACGGCATCAAGCCTGTGGTGTCCACCCAGCTGCTGCTGAACGGCTCCCT
GGCCGAGGAGGAGGTAATCATCAGGTCTGAGAACATCACAAATAACGCCAAGAA
CATCCTGGTGCAGCTGAACACCCCAGTGCAGATCAACTGTACCCGGCCTAACAAT
AATACCGTGAAGTCTATCCGGATCGGCCCAGGCCAGGCCTTCTACTATACCGGCG
ATATCATCGGCGATATCAGACAGGCCCACTGCAACGTGTCCAAGGCCACATGGA
ACGAGACACTGGGCAAGGTGGTGAAGCAGCTGCGGAAGCACTTTGGCAATAACA
CCATCATCAGATTCGCCCAGTCTTCCGGCGGCGACCTGGAGGTGACAACCCACTC
CTTCAATTGCGGCGGCGAGTTCTTTTACTGTAATACAAGCGGCCTGTTTAATAGC
ACCTGGATCTCTAACACCTCCGTGCAGGGCTCCACAGCACAGGCTCTAATGATT
CCATCACCCTGCCTTGCCGGATCAAGCAGATCATCAATATGTGGCAGAGAATCGG
CCAGGCCATGTATGCCCCTCCAATCCAGGGCGTGATCCGCTGCGTGTCCAACATC
ACAGGCCTGATCCTGACAAGAGATGGCGGCTCCACCAACAGCACCACAGAGACC
TTCAGACCCGGCGGCGGCGACATGCGCGACAACTGGAGATCCGAGCTGTATAAG
TACAAGGTGGTGAAGATCGAGCCCCTGGGCGTGGCCCCAACCCGGTGTAAGCGC
AGAGTGGTGGGCAGCCACAGCGGCAGCGGCGGCAGCGGCTCCGGCGGCCACGCC
GCCGTGGGCATCGGCGCCGTGTCCCTGGGCTTCCTGGGCGCCGCCGGCTCCACCA
TGGGCGCCGCCTCCATGACACTGACAGTGCAGGCCAGAAATCTGCTGTCCGGCAT
CGTGCAGCAGCAGTCCAATCTGCTGCGGGCCCCTGAGCCACAGCAGCACCTGCT
GAAGGATACCCACTGGGGCATCAAGCAGCTGCAGGCCCGGGTGCTGGCCGTGGA
GCACTACCTGAGGGATCAGCAGCTGCTGGGCATCTGGGGCTGTTCCGGCAAGCT
GATCTGCTGTACAAACGTGCCCTGGAACAGCTCCTGGTCCAATAGGAACCTGTCC
GAGATCTGGGATAACATGACCTGGCTGCAGTGGGATAAGGAGATCAGCAACTAC
ACACAGATCATCTACGGCCTGCTGGAGGAGAGCCAGAATCAGCAGGAGAAGAAC
GAGCAGGACCTGCTGGCCCTGGAT
BG505_SOSIP_MD39_trimer string 2 (TS2)
ATGGACTGGACATGGATTCTGTTCCTGGTCGCTGCCGCTACAAGAGTGCAT
TCCGCCGAAAACCTGTGGGTCACCGTCTACTATGGAGTGCCCGTGTGGAAGGAC
GCCGAGACTACGCTGTTCTGCGCCAGCGATGCCAAGGCCTACGAGACAGAGAAG
CACAACGTGTGGGCAACCCACGCATGCGTGCCTACAGACCCAAACCCCCAGGAG
ATCCACCTGGAGAATGTGACAGAGGAGTTTAACATGTGGAAGAACAATATGGTG
GAGCAGATGCACGAGGACATCATCTCCCTGTGGGATCAGTCTCTGAAGCCCTGCG
TGAAGCTGACCCCTCTGTGCGTGACACTGCAGTGTACCAACGTGACAAACAATAT
CACCGACGATATGCGGGGCGAGCTGAAGAATTGTAGCTTCAACATGACCACAGA
GCTGAGGGACAAGAAGCAGAAGGTGTACTCCCTGTTTTATAGACTGGATGTGGT
GCAGATCAATGAGAACCAGGGCAATCGGTCTAACAATAGCAACAAGGAGTACCG
CCTGATCAATTGCAACACCTCCGCCATCACACAGGCCTGTCCTAAGGTGTCTTTC
GAGCCTATCCCAATCCACTATTGCGCCCCAGCCGGCTTCGCCATCCTGAAGTGTA
AGGATAAGAAGTTTAACGGAACCGGACCATGCCCTTCCGTGTCTACCGTGCAGTG
TACACACGGCATCAAGCCTGTGGTGTCTACACAGCTGCTGCTGAATGGCAGCCTG
GCCGAGGAGGAAGTGATCATCAGGTCTGAGAACATCACCAACAATGCCAAGAAT
ATCCTGGTGCAGCTGAACACACCAGTGCAGATCAATTGCACCCGGCCCAACAAT
AACACAGTGAAGTCTATCCGCATCGGCCCAGGCCAGGCCTTTTACTATACCGGCG
ACATCATCGGCGATATCAGACAGGCCCACTGTAATGTGAGCAAGGCCACCTGGA
ACGAGACACTGGGCAAGGTGGTGAAGCAGCTGAGGAAGCACTTCGGCAATAACA
CCATCATCAGATTTGCACAGAGCTCCGGCGGCGACCTGGAGGTGACCACACACT
CCTTCAATTGCGGCGGCGAGTTCTTTTACTGTAACACAAGCGGCCTGTTTAATTCC
ACCTGGATCTCCAACACATCTGTGCAGGGCAGCAATTCCACCGGCAGCAACGATT
CCATCACACTGCCATGCCGGATCAAGCAGATCATCAACATGTGGCAGCGCATCG
GCCAGGCCATGTATGCCCCTCCCATCCAGGGCGTGATCAGATGCGTGAGCAATAT
CACCGGCCTGATCCTGACACGCGACGGCGGCTCTACCAACAGCACCACAGAGAC
ATTCCGGCCCGGCGGCGGCGACATGAGGGATAACTGGAGATCTGAGCTGTACAA
GTATAAGGTGGTGAAGATCGAGCCTCTGGGAGTGGCACCAACCAGGTGCAAGAG
GAGAGTGGTGGGCTCTCACAGCGGCTCCGGCGGCTCTGGCAGCGGCGGCCACGC
CGCAGTGGGCATCGGAGCCGTGTCCCTGGGCTTTCTGGGAGCAGCAGGCTCCAC
AATGGGAGCAGCCTCTATGACCCTGACAGTGCAGGCCAGGAATCTGCTGAGCGG
CATCGTGCAGCAGCAGTCCAACCTGCTGAGAGCCCCAGAGCCCCAGCAGCACCT
GCTGAAGGACACCCACTGGGGCATCAAGCAGCTGCAGGCCAGGGTGCTGGCAGT
GGAGCACTATCTGAGAGATCAGCAGCTGCTGGGCTCTGGGGCTGTAGCGGCAA
GCTGATCTGCTGTACCAATGTGCCCTGGAACTCTAGCTGGTCTAATCGCAACCTG
AGCGAGATCTGGGACAATATGACCTGGCTGCAGTGGGATAAGGAGATCTCCAAC
TACACACAGATCATCTATGGCCTGCTGGAAGAATCTCAGAATCAGCAGGAAAAG
AATGAACAGGATCTGCTGGCACTGGATggcggcagcggcagcggcGCCGAAAACCTGTGG
GTCACCGTGTACTACGGAGTCCCCGTGTGGAAAGATGCAGAGACAACCCTGTTCT
GCGCTTCCGACGCTAAAGCTTACGAGACAGAAAAACACAACGTGTGGGCCACTC
ATGCCTGCGTGCCTACAGACCCTAACCCACAGGAAATCCACCTGGAGAATGTGA
CGGAGGAGTTTAACATGTGGAAGAATAACATGGTCGAGCAGATGCATGAAGAT
TCATTTCCTTATGGGACCAATCCCTGAAGCCTTGCGTGAAGCTGACCCCACTGTG
CGTGACACTGCAATGCACTAACGTGACCAATAACATTACCGACGATATGCGCGG
CGAGCTGAAGAACTGCTCTTTCAACATGACTACCGAGCTGAGAGATAAGAAACA
GAAAGTGTACAGCCTGTTTTATCGGTTAGATGTGGTGCAGATCAATGAAAACCAG
GGCAATCGGTCCAACAATTCTAACAAGGAATATCGCCTGATCAATTGTAACACCT
CCGCCATTACCCAGGCTTGCCCTAAGGTGTCTTTCGAGCCCATCCCTATCCACTAT
TGCGCCCCAGCTGGATTTGCTATCCTGAAGTGTAAGGACAAAAAGTTTAACGGG
ACCGGACCATGTCCTAGCGTGTCCACTGTGCAGTGCACCCATGGCATCAAGCCTG
TGGTGTCCACCCAACTTCTGCTGAATGGCTCTCTGGCTGAAGAAGAAGTGATCAT
TAGGTCCGAAAATATTACTAATAACGCTAAAAATATCCTGGTCCAGCTGAACACG
CCTGTCCAGATCAATTGTACCCGGCCAAATAACAACACAGTGAAGTCTATCAGA
ATCGGCCCAGGCCAGGCCTTCTACTACACAGGCGACATTATCGGCGATATTCGCC
AGGCCCACTGTAATGTGAGCAAAGCTACATGGAATGAGACACTGGGCAAGGTAG
TCAAACAGCTGAGAAAACATTTTGGAAACAACACCATCATCCGCTTTGCACAGTC
TAGCGGCGGCGACCTGGAGGTAACTACCCACAGCTTCAATTGTGGCGGCGAGTT
CTTTTACTGTAATACCAGCGGCCTGTTTAATAGTACTTGGATCAGCAACACATCT
GTGCAGGGCTCTAACTCCACTGGCTCTAACGATAGCATCACACTGCCTTGTCGGA
TCAAGCAAATCATCAACATGTGGCAAAGGATTGGGCAGGCTATGTATGCCCCTCC
AATCCAGGGCGTGATCCGGTGCGTGAGCAACATTACAGGCCTGATCCTGACAAG
AGACGGCGGCTCCACCAACTCTACTACCGAGACATTCCGGCCCGGCGGCGGCGA
CATGCGTGATAACTGGCGCAGCGAACTGTATAAATATAAAGTGGTGAAGATCGA
GCCTCTGGGCGTGGCCCCAACTACCTGTAAAAGAAGGGTCGTCGGCTCCCACAG
CGGCAGCGGCGGCTCCGGCTCTGGCGGCCACGCGGCTGTCGGCATCGGCGCCGT
GAGCCTGGGCTTTCTGGGCGCCGCCGGCTCCACTATGGGCGCAGCCTCTATGACC
CTGACTGTCCAGGCTAGAAATCTGCTGTCTGGAATCGTGCAGCAGCAGTCTAACC
TGCTGAGGGCACCTGAGCCACAACAGCACCTGCTGAAGGATACACATTGGGGCA
TCAAGCAGTTACAAGCCAGGGTGCTGGCCGTGGAACACTACCTGCGCGATCAGC
AATTACTGGGCATTTGGGGATGCTCTGGCAAGCTGATTTGTTGCACCAATGTGCC
CTGGAACTCCTCTTGGAGCAACAGAAACCTGTCCGAAATCTGGGATAACATGAC
ATGGCTGCAGTGGGACAAGGAAATTTCCAATTATACCCAGATCATCTATGGACTG
CTGGAAGAAAGTCAGAATCAGCAGGAGAAGAATGAACAGGATCTGCTGGCACTG
GATggcggcagcggcagcggcGCCGAAAACCTGTGGGTCACCGTGTATTATGGAGTGCCA
GTGTGGAAGGACGCCGAGACCACACTGTTTTGTGCCTCTGATGCCAAGGCCTACG
AGACCGAGAAGCACAACGTGTGGGCCACCCACGCCTGCGTGCCCACAGACCCAA
ATCCTCAGGAGATCCACCTGGAGAACGTGACCGAGGAGTTTAACATGTGGAAGA
ACAATATGGTGGAGCAGATGCACGAGGATATCATCTCTCTGTGGGATCAGTCTCT
GAAGCCATGTGTGAAGCTGACCCCACTGTGCGTGACCCTGCAGTGTACAAATGTG
ACAAACAACATCACAGATGACATGAGAGGCGAGCTGAAGAACTGTTCCTTCAAT
ATGACCACCGAGCTGAGAGACAAGAAGCAGAAGGTGTATTCTCTGTTTTACCGG
CTGGACGTGGTGCAGATCAACGAGAATCAGGGCAATCGGTCTAACAACTCCAAT
AAGGAGTATAGACTGATCAACTGCAACACCTCTGCCATCACCCAGGCCTGTCCTA
AGGTGTCCTTTGAGCCAATCCCAATCCACTATTGCGCCCCTGCCGGCTTTGCCATC
CTGAAGTGCAAGGACAAGAAGTTTAACGGCACAGGCCCCTGCCCATCCGTGAGC
ACAGTGCAGTGTACCCACGGCATCAAGCCTGTGGTGTCCACCCAGCTGCTGCTGA
ACGGCTCCCTGGCCGAGGAGGAGGTAATCATCAGGTCTGAGAACATCACAAATA
ACGCCAAGAACATCCTGGTGCAGCTGAACACCCCAGTGCAGATCAACTGTACC
GGCCTAACAATAATACCGTGAAGTCTATCCGGATCGGCCCAGGCCAGGCCTTCTA
CTATACCGGCGATATCATCGGCGATATCAGACAGGCCCACTGCAACGTGTCCAA
GGCCACATGGAACGAGACACTGGGCAAGGTGGTGAAGCAGCTGCGGAAGCACTT
TGGCAATAACACCATCATCAGATTCGCCCAGTCTTCCGGCGGCGACCTGGAGGTG
ACAACCCACTCCTTCAATTGCGGCGGCGAGTTCTTTTACTGTAATACAAGCGGCC
TGTTTAATAGCACCTGGATCTCTAACACCTCCGTGCAGGGCTCCAACAGCACAGG
CTCTAATGATTCCATCACCCTGCCTTGCCGGATCAAGCAGATCATCAATATGTGG
CAGAGAATCGGCCAGGCCATGTATGCCCCTCCAATCCAGGGCGTGATCCGCTGC
GTGTCCAACATCACAGGCCTGATCCTGACAAGAGATGGCGGCTCCACCAACAGC
ACCACAGAGACCTTCAGACCCGGCGGCGGCGACATGCGCGACAACTGGAGTCC
GAGCTGTATAAGTACAAGGTGGTGAAGATCGAGCCCCTGGGCGTGGCCCCAACC
CGGTGTAAGCGCAGAGTGGTGGGCAGCCACAGCGGCAGCGGCGGCAGCGGCTCC
GGCGGCCACGCCGCCGTGGGCATCGGCGCCGTGTCCCTGGGCTTCCTGGGCGCCG
CCGGCTCCACCATGGGCGCCGCCTCCATGACACTGACAGTGCAGGCCAGAAATC
TGCTGTCCGGCATCGTGCAGCAGCAGTCCAATCTGCTGCGGGCCCCTGAGCCACA
GCAGCACCTGCTGAAGGATACCCACTGGGGCATCAAGCAGCTGCAGGCCCGGGT
GCTGGCCGTGGAGCACTACCTGAGGGATCAGCAGCTGCTGGGCATCTGGGGCTG
TTCCGGCAAGCTGATCTGCTGTACAAACGTGCCCTGGAACAGCTCCTGGTCCAAT
AGGAACCTGTCCGAGATCTGGGATAACATGACCTGGCTGCAAGTGGGATAAGGAG
ATCAGCAACTACACACAGATCATCTACGGCCTGCTGGAGGAGAGCCAGAATCAG
CAGGAGAAGAACGAGCAGGACCTGCTGGCCCTGGATTGATAA
BG505_MD39_link14_gp140-PDGFR
ATGGACTGGACATGGATTCTGTTCCTGGTCGCTGCCGCTACAAGAGTGCAT
TCCGCCGAAAACCTGTGGGTCACCGTCTACTATGGAGTGCCCGTGTGGAAGGAC
GCCGAGACTACGCTGTTCTGCGCCAGCGATGCCAAGGCCTACGAGACAGAGAAG
CACAACGTGTGGGCAACCCACGCATGCGTGCCTACAGACCCAAACCCCCAGGAG
ATCCACCTGGAGAATGTGACAGAGGAGTTTAACATGTGGAAGAACAATATGGTG
GAGCAGATGCACGAGGACATCATCTCCCTGTGGGATCAGTCTCTGAAGCCCTGCG
TGAAGCTGACCCCTCTGTGCGTGACACTGCAGTGTACCAACGTGACAAACAATAT
CACCGACGATATGCGGGGCGAGCTGAAGAATTGTAGCTTCAACATGACCACAGA
GCTGAGGGACAAGAAGCAGAAGGTGTACTCCCTGTTTTATAGACTGGATGTGGT
GCAGATCAATGAGAACCAGGGCAATCGGTCTAACAATAGCAACAAGGAGTACCG
CCTGATCAATTGCAACACCTCCGCCATCACACAGGCCTGTCCTAAGGTGTCTTTC
GAGCCTATCCCAATCCACTATTGCGCCCCAGCCGGCTTCGCCATCCTGAAGTGTA
AGGATAAGAAGTTTAACGGAACCGGACCATGCCCTTCCGTGTCTACCGTGCAGTG
TACACACGGCATCAAGCCTGTGGTGTCTACACAGCTGCTGCTGAATGGCAGCCTG
GCCGAGGAGGAAGTGATCATCAGGTCTGAGAACATCACCAACAATGCCAAGAAT
ATCCTGGTGCAGCTGAACACACCAGTGCAGATCAATTGCACCCGGCCCAACAAT
AACACAGTGAAGTCTATCCGCATCGGCCCAGGCCAGGCCTTTTACTATACCGGCG
ACATCATCGGCGATATCAGACAGGCCCACTGTAATGTGAGCAAGGCCACCTGGA
ACGAGACACTGGGCAAGGTGGTGAAGCAGCTGAGGAAGCACTTCGGCAATAACA
CCATCATCAGATTTGCACAGAGCTCCGGCGGCGACCTGGAGGTGACCACACACT
CCTTCAATTGCGGCGGCGAGTTCTTTTACTGTAACACAAGCGGCCTGTTTAATTCC
ACCTGGATCTCCAACACATCTGTGCAGGGCAGCAATTCCACCGGCAGCAACGATT
CCATCACACTGCCATGCCGGATCAAGCAGATCATCAACATGTGGCAGCGCATCG
GCCAGGCCATGTATGCCCCTCCCATCCAGGGCGTGATCAGATGCGTGAGCAATAT
CACCGGCCTGATCCTGACACGCGACGGCGGCTCTACCAACAGCACCACAGAGAC
ATTCCGGCCCGGCGGCGGCGACATGAGGGATAACTGGAGATCTGAGCTGTACAA
GTATAAGGTGGTGAAGATCGAGCCTCTGGGAGTGGCACCAACCAGGTGCAAGAG
GAGAGTGGTGGGCTCTCACAGCGGCTCCGGCGGCTCTGGCAGCGGCGGCCACGC
CGCAGTGGGCATCGGAGCCGTGTCCCTGGGCTTTCTGGGAGCAGCAGGCTCCAC
AATGGGAGCAGCCTCTATGACCCTGACAGTGCAGGCCAGGAATCTGCTGAGCGG
CATCGTGCAGCAGCAGTCCAACCTGCTGAGAGCCCCAGAGCCCCAGCAGCACCT
GCTGAAGGACACCCACTGGGGCATCAAGCAGCTGCAGGCCAGGGTGCTGGCAGT
GGAGCACTATCTGAGAGATCAGCAGCTGCTGGGCATCTGGGGCTGTAGCGGCAA
GCTGATCTGCTGTACCAATGTGCCCTGGAACTCTAGCTGGTCTAATCGCAACCTG
AGCGAGATCTGGGACAATATGACCTGGCTGCAGTGGGATAAGGAGATCTCCAAC
TACACACAGATCATCTATGGCCTGCTGGAAGAATCTCAGAATCAGCAGGAAAAG
AATGAACAGGATCTGCTGGCACTGGATGGAGGAGGAAGCGGGGGAAGCGGGGG
AAGCGGAGGAAGCGGGGGAAGCGGGGGAAGCAACGCCGTGGGCCAGGACACCC
AGGAAGTGATCGTGGTGCCCCACAGCCTGCCTTTCAAGGTGGTGGTCATCTCCGC
CATCCTGGCCCTGGTCGTGCTGACTATTATTTCCCTGATTATCCTGATTATGCTGT
GGCAGAAGAAGCCCAGATGATAA
BG505_MD39_gp140_foldon-PDGFR
ATGGACTGGACATGGATTCTGTTCCTGGTCGCTGCCGCTACAAGAGTGCAT
TCCGCCGAAAACCTGTGGGTCACCGTCTCTATGGAGTGCCCGTGTGGAAGGAC
GCCGAGACTCGCTTTCTGCGCCAGCGATGCCAAGGCCTACGAGACAGAGAAG
CACAACGTGTGGGCAACCCACGCATGCGTGCCTACAGACCCAAACCCCCAGGAG
ATCCACCTGGAGAATGTGACAGAGGAGTTTAACATGTGGAAGAACAATATGGTG
GAGCAGATGCACGAGGACATCATCTCCCTGTGGGATCAGTCTCTGAAGCCCTGCG
TGAAGCTGACCCCTCTGTGCGTGACACTGCAGTGTACCAACGTGACAAACAATAT
CACCGACGATATGCGGGGCGAGCTGAAGAATTGTAGCTTCAACATGACCACAGA
GCTGAGGGACAAGAAGCAGAAGGTGTACTCCCTGTTTTATAGACTGGATGTGGT
GCAGATCAATGAGAACCAGGGCAATCGGTCTAACAATAGCAACAAGGAGTACCG
CCTGATCAATTGCAACACCTCCGCCATCACACAGGCCTGTCCTAAGGTGTCTTTC
GAGCCTATCCCAATCCACTATTGCGCCCCAGCCGGCTTCGCCATCCTGAAGTGTA
AGGATAAGAAGTTTAACGGAACCGGACCATGCCCTTCCGTGTCTACCGTGCAGTG
TACACACGGCATCAAGCCTGTGGTGTCTACACAGCTGCTGCTGAATGGCAGCCTG
GCCGAGGAGGAAGTGATCATCAGGTCTGAGAACATCACCAACAATGCCAAGAAT
ATCCTGGTGCAGCTGAACACACCAGTGCAGATCAATTGCACCCGGCCCAACAAT
AACACAGTGAAGTCTATCCGCATCGGCCCAGGCCAGGCCTTTTACTATACCGGCG
ACATCATCGGCGATATCAGACAGGCCCACTGTAATGTGAGCAAGGCCACCTGGA
ACGAGACACTGGGCAAGGTGGTGAAGCAGCTGAGGAAGCACTTCGGCAATAACA
CCATCATCAGATTTGCACAGAGCTCCGGCGGCGACCTGGAGGTGACCACACACT
CCTTCAATTGCGGCGGCGAGTTCTTTTACTGTAACACAAGCGGCCTGTTTAATTCC
ACCTGGATCTCCAACACATCTGTGCAGGGCAGCAATTCCACCGGCAGCAACGATT
CCATCACACTGCCATGCCGGATCAAGCAGATCATCAACATGTGGCAGCGCATCG
GCCAGGCCATGTATGCCCCTCCCATCCAGGGCGTGATCAGATGCGTGAGCAATAT
CACCGGCCTGATCCTGACACGCGACGGCGGCTCTACCAACAGCACCACAGAGAC
ATTCCGGCCCGGCGGCGGCGACATGAGGGATAACTGGAGATCTGAGCTGTACAA
GTATAAGGTGGTGAAGATCGAGCCTCTGGGAGTGGCACCAACCAGGTGCAAGAG
GAGAGTGGTGGGCTCTCACAGCGGCTCCGGCGGCTCTGGCAGCGGCGGCCACGC
CGCAGTGGGCATCGGAGCCGTGTCCCTGGGCTTTCTGGGAGCAGCAGGCTCCAC
AATGGGAGCAGCCTCTATGACCCTGACAGTGCAGGCCAGGAATCTGCTGAGCGG
CATCGTGCAGCAGCAGTCCAACCTGCTGAGAGCCCCAGAGCCCCAGCAGCACCT
GCTGAAGGACACCCACTGGGGCATCAAGCAGCTGCAGGCCAGGGTGCTGGCAGT
GGAGCACTATCTGAGAGATCAGCAGCTGCTGGGCATCTGGGGCTGGTAGCGGCAA
GCTGATCTGCTGTACCAATGTGCCCTGGAACTCTAGCTGGTCTAATCGCAACCTG
AGCGAGATCTGGGACAATATGACCTGGCTGCAGTGGGATAAGGAGATCTCCAAC
TACACACAGATCATCTATGGCCTGCTGGAAGAATCTCAGAATCAGCAGGAAAAG
AATGAACAGGATCTGCTGGCACTGGATGGAGGAGGAAGCGGGGGAAGCGGCGG
CGGCTACATCCCTGAGGCCCCAAGGGACGGACAGGCCTATGTGAGAAAGGATGG
CGAGTGGGTGCTGCTGTCCACCTTCCTGGGGGAAGCGGAGGAAGCGGGGGAAG
CGGGGGAAGCAACGCCGTGGGCCAGGACACCCAGGAAGTGATCGTGGTGCCCCA
CAGCCTGCCTTTCAAGGTGGTGGTCATCTCCGCCATCCTGGCCCTGGTCGTGCTG
ACTATTATTTCCCTGATTATCCTGATTATGCTGTGGCAGAAGAAGCCCAGATGAT
AA
BG505_MD39_TS1_gp140-PDGFR
ATGGACTGGACATGGATTCTGTTCCTGGTCGCTGCCGCTACAAGAGTGCAT
TCCGCCGAAAACCTGTGGGTCACCGTCTACTATGGAGTGCCCGTGTGGAAGGAC
GCCGAGACTACGCTGTTCTGCGCCAGCGATGCCAAGGCCTACGAGACAGAGAAG
CACAACGTGTGGGCAACCCACGCATGCGTGCCTACAGACCCAAACCCCCAGGAG
ATCCACCTGGAGAATGTGACAGAGGAGTTTAACATGTGGAAGAACAATATGGTG
GAGCAGATGCACGAGGACATCATCTCCCTGTGGGATCAGTCTCTGAAGCCCTGCG
TGAAGCTGACCCCTCTGTGCGTGACACTGCAGTGTACCAACGTGACAAACAATAT
CACCGACGATATGCGGGGCGAGCTGAAGAATTGTAGCTTCAACATGACCACAGA
GCTGAGGGACAAGAAGCAGAAGGTGTACTCCCTGTTTTATAGACTGGATGTGGT
GCAGATCAATGAGAACCAGGGCAATCGGTCTAACAATAGCAACAAGGAGTACCG
CCTGATCAATTGCAACACCTCCGCCATCACACAGGCCTGTCCTAAGGTGTCTTTC
GAGCCTATCCCAATCCACTATTGCGCCCCAGCCGGCTTCGCCATCCTGAAGTGTA
AGGATAAGAAGTTTAACGGAACCGGACCATGCCCTTCCGTGTCTACCGTGCAGTG
TACACACGGCATCAAGCCTGTGGTGTCTACACAGCTGCTGCTGAATGGCAGCCTG
GCCGAGGAGGAAGTGATCATCAGGTCTGAGAACATCACCAACAATGCCAAGAAT
ATCCTGGTGCAGCTGAACACACCAGTGCAGATCAATTGCACCCGGCCCAACAAT
AACACAGTGAAGTCTATCCGCATCGGCCCAGGCCAGGCCTTTTACTATACCGGCG
ACATCATCGGCGATATCAGACAGGCCCACTGTAATGTGAGCAAGGCCACCTGGA
ACGAGACACTGGGCAAGGTGGTGAAGCAGCTGAGGAAGCACTTCGGCAATAACA
CCATCATCAGATTTGCACAGAGCTCCGGCGGCGACCTGGAGGTGACCACACACT
CCTTCAATTGCGGCGGCGAGTTCTTTTACTGTAACACAAGCGGCCTGTTTAATTCC
ACCTGGATCTCCAACACATCTGTGCAGGGCAGCAATTCCACCGGCAGCAACGATT
CCATCACACTGCCATGCCGGATCAAGCAGATCATCAACATGTGGCAGCGCATCG
GCCAGGCCATGTATGCCCCTCCCATCCAGGGCGTGATCAGATGCGTGAGCAATAT
CACCGGCCTGATCCTGACACGCGACGGCGGCTCTACCAACAGCACCACAGAGAC
ATTCCGGCCCGGCGGCGGCGACATGAGGGATAACTGGAGATCTGAGCTGTACAA
GTATAAGGTGGTGAAGATCGAGCCTCTGGGAGTGGCACCAACCAGGTGCAAGAG
GAGAGTGGTGGGCTCTCACAGCGGCTCCGGCGGCTCTGGCAGCGGCGGCCACGC
CGCAGTGGGCATCGGAGCCGTGTCCCTGGGCTTTCTGGGAGCAGCAGGCTCCAC
AATGGGAGCAGCCTCTATGACCCTGACAGTGCAGGCCAGGAATCTGCTGAGCGG
CATCGTGCAGCAGCAGTCCAACCTGCTGAGAGCCCCAGAGCCCCAGCAGCACCT
GCTGAAGGACACCCACTGGGGCATCAAGCAGCTGCAGGCCAGGGTGCTGGCAGT
GGAGCACTATCTGAGAGATCAGCAGCTGCTGGGCATCTGGGGCTGTAGCGGCAA
GCTGATCTGCTGTACCAATGTGCCCTGGAACTCTAGCTGGTCTAATCGCAACCTG
AGCGAGATCTGGGACAATATGACCTGGCTGCAGTGGGATAAGGAGATCTCCAAC
TACACACAGATCATCTATGGCCTGCTGGAAGAATCTCAGAATCAGCAGGAAAAG
AATGAACAGGATCTGCTGGCACTGGATGGCGGCGCCGAAAACCTGTGGGTCACC
GTGTACTACGGAGTCCCCGTGTGGAAAGATGCAGAGACAACCCTGTTCTGCGCTT
CCGACGCTAAAGCTTACGAGACAGAAAAACACAACGTGTGGGCCACTCATGCCT
GCGTGCCTACAGACCCTAACCCACAGGAAATCCACCTGGAGAATGTGACGGAGG
AGTTTAACATGTGGAAGAATAACATGGTCGAGCAGATGCATGAAGATATCATTT
CCTTATGGGACCAATCCCTGAAGCCTTGCGTGAAGCTGACCCCACTGTGCGTGAC
ACTGCAATGCACTAACGTGACCAATAACATTACCGACGATATGCGCGGCGAGCT
GAAGAACTGCTCTTTCAACATGACTACCGAGCTGAGAGATAAGAAACAGAAAGT
GTACAGCCTGTTTTATCGGTTAGATGTGGTGCAGATCAATGAAAACCAGGGCAAT
CGGTCCAACAATTCTAACAAGGAATATCGCCTGATCAATTGTAACACCTCCGCCA
TTACCCAGGCTTGCCCTAAGGTGTCTTTCGAGCCCATCCCTATCCACTATTGCGCC
CCAGCTGGATTTGCTATCCTGAAGTGTAAGGACAAAAGTTTAACGGGACCGGA
CCATGTCCTAGCGTGTCCACTGTGCAGTGCACCCATGGCATCAAGCCTGTGGTGT
CCACCCAACTTCTGCTGAATGGCTCTCTGGCTGAAGAAGAAGTGATCATTAGGTC
CGAAAATATTACTAATAACGCTAAAAATATCCTGGTCCAGCTGAACACGCCTGTC
CAGATCAATTGTACCCGGCCAAATAACAACACAGTGAAGTCTATCAGAATCGGC
CCAGGCCAGGCCTTCTACTACACAGGCGACATTATCGGCGATATTCGCCAGGCCC
ACTGTAATGTGAGCAAAGCTACATGGAATGAGACACTGGGCAAGGTAGTCAAAC
AGCTGAGAAAACATTTTGGAAACAACACCATCATCCGCTTTGCACAGTCTAGCGG
CGGCGACCTGGAGGTAACTACCCACAGCTTCAATTGTGGCGGCGAGTTCTTTTAC
TGTAATACCAGCGGCCTGTTTAATAGTACTTGGATCAGCAACACATCTGTGCAGG
GCTCTAACTCCACTGGCTCTAACGATAGCATCACACTGCCTTGTCGGATCAAGCA
AATCATCAACATGTGGCAAAGGATTGGGCAGGCTATGTATGCCCCTCCAATCCAG
GGCGTGATCCGGTGCGTGAGCAACATTACAGGCCTGATCCTGACAAGAGACGGC
GGCTCCACCAACTCTACTACCGAGACATTCCGGCCCGGCGGCGGCGACATGCGT
GATAACTGGCGCAGCGAACTGTATAAATATAAAGTGGTGAAGATCGAGCCTCTG
GGCGTGGCCCCAACTAGGTGTAAAAGAAGGGTCGTCGGCTCCCACAGCGGCAGC
GGCGGCTCCGGCTCTGGCGGCCACGCGGCTGTCGGCATCGGCGCCGTGAGCCTG
GGCTTTCTGGGCGCCGCCGGCTCCACTATGGGCGCAGCCTCTATGACCCTGACTG
TCCAGGCTAGAAATCTGCTGTCTGGAATCGTGCAGCAGCAGTCTAACCTGCTGAG
GGCACCTGAGCCACAACAGCACCTGCTGAAGGATACACATTGGGGCATCAAGCA
GTTACAAGCCAGGGTGCTGGCCGTGGAACACTACCTGCGCGATCAGCAATTACT
GGGCATTTGGGGATGCTCTGGCAAGCTGATTTGTTGCACCAATGTGCCCTGGAAC
TCCTCTTGGAGCAACAGAAACCTGTCCGAAATCTGGGATAACATGACATGGCTGC
AGTGGGACAAGGAAATTTCCAATTATACCCAGATCATCTATGGACTGCTGGAAG
AAAGTCAGAATCAGCAGGAGAAGAATGAACAGGATCTGCTGGCACTGGATGGCG
GCGCCGAAAACCTGTGGGTCACCGTGTATTATGGAGTGCCAGTGTGGAAGGACG
CCGAGACCACACTGTTTTGTGCCTCTGATGCCAAGGCCTACGAGACCGAGAAGC
ACAACGTGTGGGCCACCCACGCCTGCGTGCCCACAGACCCAAATCCTCAGGAGA
TCCACCTGGAGAACGTGACCGAGGAGTTTAACATGTGGAAGAACAATATGGTGG
AGCAGATGCACGAGGATATCATCTCTCTGTGGGATCAGTCTCTGAAGCCATGTGT
GAAGCTGACCCCACTGTGCGTGACCCTGCAGTGTACAAATGTGACAAACAACAT
CACAGATGACATGAGAGGCGAGCTGAAGAACTGTTCCTTCAATATGACCACCGA
GCTGAGAGACAAGAAGCAGAAGGTGTATTCTCTGTTTTACCGGCTGGACGTGGT
GCAGATCAACGAGAATCAGGGCAATCGGTCTAACAACTCCAATAAGGAGTATAG
ACTGATCAACTGCAACACCTCTGCCATCACCCAGGCCTGTCCTAAGGTGTCCTTT
GAGCCAATCCCAATCCACTATTGCGCCCCTGCCGGCTTTGCCATCCTGAAGTGCA
AGGACAAGAAGTTTAACGGCACAGGCCCCTGCCCATCCGTGAGCACAGTGCAGT
GTACCCACGGCATCAAGCCTGTGGTGTCCACCCAGCTGCTGCTGAACGGCTCCCT
GGCCGAGGAGGAGGTAATCATCAGGTCTGAGAACATCACAAATAACGCCAAGAA
CATCCTGGTGCAGCTGAACACCCCAGTGCAGATCAACTGTACCCGGCCTAACAAT
AATACCGTGAAGTCTATCCGGATCGGCCCAGGCCAGGCCTTCTACTATACCGGCG
ATATCATCGGCGATATCAGACAGGCCCACTGCAACGTGTCCAAGGCCACATGGA
ACGAGACACTGGGCAAGGTGGTGAAGCAGCTGCGGAAGCACTTTGGCAATAACA
CCATCATCAGATTCGCCCAGTCTTCCGGCGGCGACCTGGAGGTGACAACCCACTC
CTTCAATTGCGGCGGCGAGTTCTTTTACTGTAATACAAGCGGCCTGTTTAATAGC
ACCTGGATCTCTAACACCTCCGTGCAGGGCTCCAACAGCACAGGCTCTAATGATT
CCATCACCCTGCCTTGCCGGATCAAGCAGATCATCAATATGTGGCAGAGAATCGG
CCAGGCCATGTATGCCCCTCCAATCCAGGGCGTGATCCGCTGCGTGTCCAACATC
ACAGGCCTGATCCTGACAAGAGATGGCGGCTCCACCAACAGCACCACAGAGACC
TTCAGACCCGGCGGCGGCGACATGCGCGACAACTGGAGATCCGAGCTGTATAAG
TACAAGGTGGTGAAGATCGAGCCCCTGGGCGTGGCCCCAACCCGGTGTAAGCGC
AGAGTGGTGGGCAGCCACAGCGGCAGCGGCGGCAGCGGCTCCGGCGGCCACGCC
GCCGTGGGCATCGGCGCCGTGTCCCTGGGCTTCCTGGGCGCCGCCGGCTCCACCA
TGGGCGCCGCCTCCATGACACTGACAGTGCAGGCCAGAAATCTGCTGTCCGGCAT
CGTGCAGCAGCAGTCCAATCTGCTGCGGGCCCCTGAGCCACAGCAGCACCTGCT
GAAGGATACCCACTGGGGCATCAAGCAGCTGCAGGCCCGGGTGCTGGCCGTGGA
GCACTACCTGAGGGATCAGCAGCTGCTGGGCATCTGGGGCTGTTCCGGCAAGCT
GATCTGCTGTACAAACGTGCCCTGGAACAGCTCCTGGTCCAATAGGAACCTGTCC
GAGATCTGGGATAACATGACCTGGCTGCAGTGGGATAAGGAGATCAGCAACTAC
ACACAGATCATCTACGGCCTGCTGGAGGAGAGCCAGAATCAGCAGGAGAAGAAC
GAGCAGGACCTGCTGGCCCTGGATGGAGGAGGAAGCGGGGGAAGCGGGGGAAG
CGGAGGAAGCGGGGGAAGCGGGGGAAGCAACGCCGTGGGCCAGGACACCCAGG
AAGTGATCGTGGTGCCCCACAGCCTGCCTTTCAAGGTGGTGGTCATCTCCGCCAT
CCTGGCCCTGGTCGTGCTGACTATTATTTCCCTGATTATCCTGATTATGCTGTGGC
AGAAGAAGCCCAGATGATAA
(25) BG505_MD39_TS1_gp140-PDGFR
GGATCCGCCACCATGGACTGGACATGGATTCTGTTCCTGGTCGCTGCCGCT
ACAAGAGTGCATTCCGCCGAAAACCTGTGGGTCACCGTCTACTATGGAGTGCCCG
TGTGGAAGGACGCCGAGACTACGCTGTTCTGCGCCAGCGATGCCAAGGCCTACG
AGACAGAGAAGCACAACGTGTGGGCAACCCACGCATGCGTGCCTACAGACCCAA
ACCCCCAGGAGATCCACCTGGAGAATGTGACAGAGGAGTTTAACATGTGGAAGA
ACAATATGGTGGAGCAGATGCACGAGGACATCATCTCCCTGTGGGATCAGTCTCT
GAAGCCCTGCGTGAAGCTGACCCCTCTGTGCGTGACACTGCAGTGTACCAACGTG
ACAAACAATATCACCGACGATATGCGGGGCGAGCTGAAGAATTGTAGCTTCAAC
ATGACCACAGAGCTGAGGGACAAGAAGCAGAAGGTGTACTCCCGTGTTTTATAGA
CTGGATGTGGTGCAGATCAATGAGAACCAGGGCAATCGGTCTAACAATAGCAAC
AAGGAGTACCGCCTGATCAATTGCAACACCTCCGCCATCACACAGGCCTGTCCTA
AGGTGTCTTTCGAGCCTATCCCAATCCACTATTGCGCCCCAGCCGGCTTCGCCAT
CCTGAAGTGTAAGGATAAGAAGTTTAACGGAACCGGACCATGCCCTTCCGTGTCT
ACCGTGCAGTGTACACACGGCATCAAGCCTGTGGTGTCTACACAGCTGCTGCTGA
ATGGCAGCCTGGCCGAGGAGGAAGTGATCATCAGGTCTGAGAACATCACCAACA
ATGCCAAGAATATCCTGGTGCAGCTGAACACACCAGTGCAGATCAATTGCACCC
GGCCCAACAATAACACAGTGAAGTCTATCCGCATCGGCCCAGGCCAGGCCTTTTA
CTATACCGGCGACATCATCGGCGATATCAGACAGGCCCACTGTAATGTGAGCAA
GGCCACCTGGAACGAGACACTGGGCAAGGTGGTGAAGCAGCTGAGGAAGCACTT
CGGCAATAACACCATCATCAGATTTGCACAGAGCTCCGGCGGCGACCTGGAGGT
GACCACACACTCCTTCAATTGCGGCGGCGAGTTCTTTTACTGTAACACAAGCGGC
CTGTTTAATTCCACCTGGATCTCCAACACATCTGTGCAGGGCAGCAATTCCACCG
GCAGCAACGATTCCATCACACTGCCATGCCGGATCAAGCAGATCATCAACATGT
GGCAGCGCATCGGCCAGGCCATGTATGCCCCTCCCATCCAGGGCGTGATCAGAT
GCGTGAGCAATATCACCGGCCTGATCCTGACACGCGACGGCGGCTCTACCAACA
GCACCACAGAGACATTCCGGCCCGGCGGCGGCGACATGAGGGATAACTGGAGAT
CTGAGCTGTACAAGTATAAGGTGGTGAAGATCGAGCCTCTGGGAGTGGCACCAA
CCAGGTGCAAGAGGAGAGTGGTGGGCTCTCCAGCGGCTCCGGCGGCTCTGGCA
GCGGCGGCCACGCCGCAGTGGGCATCGGAGCCGTGTCCCTGGGCTTTCTGGGAG
CAGCAGGCTCCACAATGGGAGCAGCCTCTATGACCCTGACAGTGCAGGCCAGGA
ATCTGCTGAGCGGCATCGTGCAGCAGCAGTCCAACCTGCTGAGAGCCCCAGAGC
CCCAGCAGCACCTGCTGAAGGACACCCACTGGGGCATCAAGCAGCTGCAGGCCA
GGGTGCTGGCAGTGGAGCACTATCTGAGAGATCAGCAGCTGCTGGGCATCTGGG
GCTGTAGCGGCAAGCTGATCTGCTGTACCAATGTGCCCTGGAACTCTAGCTGGTC
TAATCGCAACCTGAGCGAGATCTGGGACAATATGACCTGGCTGCAGTGGGATAA
GGAGATCTCCAACTACACACAGATCATCTATGGCCTGCTGGAAGAATCTCAGAAT
CAGCAGGAAAAGAATGAACAGGATCTGCTGGCACTGGATGGCGGCGCCGAAAA
CCTGTGGGTCACCGTGTACTACGGAGTCCCCGTGTGGAAAGATGCAGAGACAAC
CCTGTTCTGCGCTTCCGACGCTAAAGCTTACGAGACAGAAAAACACAACGTGTG
GGCCACTCATGCCTGCGTGCCTACAGACCCTAACCCACAGGAAATCCACCTGGA
GAATGTGACGGAGGAGTTTAACATGTGGAAGAATAACATGGTCGAGCAGATGCA
TGAAGATATCATTTCCTTATGGGACCAATCCCTGAAGCCTTGCGTGAAGCTGACC
CCACTGTGCGTGACACTGCAATGCACTAACGTGACCAATAACATTACCGACGATA
TGCGCGGCGAGCTGAAGAACTGCTCTTTCAACATGACTACCGAGCTGAGGATA
AGAAACAGAAAGTGTACAGCCTGTTTTATCGGTTAGATGTGGTGCAGATCAATG
AAAACCAGGGCAATCGGTCCAACAATTCTAACAAGGAATATCGCCTGATCAATT
GTAACACCTCCGCCATTACCCAGGCTTGCCCTAAGGTGTCTTTCGAGCCCATCCC
TATCCACTATTGCGCCCCAGCTGGATTTGCTATCCTGAAGTGTAAGGACAAAAAG
TTTAACGGGACCGGACCATGTCCTAGCGTGTCCACTGTGCAGTGCACCCATGGCA
TCAAGCCTGTGGTGTCCACCCAACTTCTGCTGAATGGCTCTCTGGCTGAAGAAGA
AGTGATCATTAGGTCCGAAAATATTACTAATAACGCTAAAAATATCCTGGTCCAG
CTGAACACGCCTGTCCAGATCAATTGTACCCGGCCAAATAACACACAGTGAAG
TCTATCAGAATCGGCCCAGGCCAGGCCTTCTACTACACAGGCGACATTATCGGCG
ATATTCGCCAGGCCCACTGTAATGTGAGCAAAGCTACATGGAATGAGACACTGG
GCAAGGTAGTCAAACAGCTGAGAAAACATTTTGGAAACAACACCATCATCCGCT
TTGCACAGTCTAGCGGCGGCGACCTGGAGGTAACTACCCACAGCTTCAATTGTGG
CGGCGAGTTCTTTTACTGTAATACCAGCGGCCTGTTTAATAGTACTTGGATCAGC
AACACATCTGTGCAGGGCTCTAACTCCACTGGTCTAACGATAGCATCACACTGC
CTTGTCGGATCAAGCAAATCATCAACATGTGGCAAAGGATTGGGCAGGCTATGT
ATGCCCCTCCAATCCAGGGCGTGATCCGGTGCGTGAGCAACATTACAGGCCTGAT
CCTGACAAGAGACGGCGGCTCCACCAACTCTACTACCGAGACATTCCGGCCCGG
CGGCGGCGACATGCGTGATAATGGCGCAGCGAACTGTATAAATATAAAGTGGT
GAAGATCGAGCCTCTGGGCGTGGCCCCAACTAGGTGTAAAAGAAGGGTCGTCGG
CTCCCACAGCGGCAGCGGCGGCTCCGGCTCTGGCGGCCACGCGGCTGTCGGCAT
CGGCGCCGTGAGCCTGGGTTTCTGGGCGCCGCCGGCTCCACTATGGGCGCAGCC
TCTATGACCCTGACTGTCCAGGCTAGAAATCTGCTGTCTGGAATCGTGCAGCAGC
AGTCTAACCTGCTGAGGGCACCTGAGCCACAACAGCCCTGCTGAAGGATACAC
ATGGGGCATCAAGCAGTTACAAGCCAGGGTGCTGGCCGTGGAACACTACCTGC
GCGATCAGCAATTACTGGGCATTTGGGGATGCTCTGGCAAGCTGATTTGTTGCAC
CAATGTGCCCTGGAACTCCTCTTGGAGCAACAGAAACCTGTCCGAAATCTGGGAT
AACATGACATGGCTGCAGTGGGACAAGGAAATTTCCAATTATACCCAGATCATCT
ATGGACTGCTGGAAGAAAGTCAGAATCAGCAGGAGAAGAATGAACAGGATCTG
CTGGCACTGGATGGCGGCGCCGAAAACCTGTGGGTCACCGTGTATTATGGAGTG
CCAGTGTGGAAGGACGCCGAGACCACACTGTTTTGTGCCTCTGATGCCAAGGCCT
ACGAGACCGAGAAGCACAACGTGTGGGCCACCCACGCCTGCGTGCCCACAGACC
CAAATCCTCAGGAGATCCACCTGGAGAACGTGACCGAGGAGTTTAACATGTGGA
AGAACAATATGGTGGAGCAGATGCACCGAGGATATCATCTCTCTGTGGGATCAGT
CTCTGAAGCCATGTGTGAAGCTGACCCCACTGTGCGTGACCCTGCAGTGTACAAA
TGTGACAAACAACATCACAGATGACATGAGAGGCGAGCTGAAGAACTGTTCCTT
CAATATGACCACCGAGCTGAGAGACAAGAAGCAGAAGGTGTATTCTCTGTTTTA
CCGGCTGGACGTGGTGCAGATCAACGAGAATCAGGGCAATCGGTCTAACAACTC
CAATAAGGAGTATAGACTGATCAACTGCAACACCTCTGCCATCACCCAGGCCTGT
CCTAAGGTGTCCTTTGAGCCAATCCCAATCCACTATTGCGCCCCTGCCGGCTTTGC
CATCCTGAAGTGCAAGGACAAGAAGTTTAACGGCACAGGCCCCTGCCCATCCGT
GAGCACAGTGCAGTGTACCCACGGCATCAAGCCTGTGGTGTCCACCCAGCTGCTG
CTGAACGGCTCCCTGGCCGAGGAGGAGGTAATCATCAGGTCTGAGAACATCACA
AATAACGCCAAGAACATCCTGGTGCAGCTGAACACCCCAGTGCAGATCAACTGT
ACCCGGCCTAACAATAATACCGTGAAGTCTATCCGGATCGGCCCAGGCCAGGCC
TTCTACTATACCGGCGATATCATCGGCGATATCAGACAGGCCCACTGCAACGTGT
CCAAGGCCACATGGAACGAGACACTGGGCAAGGTGGTGAAGCAGCTGCGGAAG
CACTTTGGCAATAACACCATCATCAGATTCGCCCAGTCTTCCGGCGGCGACCTGG
AGGTGACAACCCACTCCTTCAATTGCGGCGGCGAGTTCTTTTACTGTAATACAAG
CGGCCTGTTTAATAGCACCTGGATCTCTAACACCTCCGTGCAGGGCTCCAACAGC
ACAGGCTCTAATGATTCCATCACCCTGCCTTGCCGGATCAAGCAGATCATCAATA
TGTGGCAGAGATCGGCCAGGCCATGTATGCCCCTCCAATCCAGGGCGTGATCC
GCTGCGTGTCCAACATCACAGGCCTGATCCTGACAAGAGATGGCGGCTCCACCA
ACAGCACCACAGAGACCTTCAGACCCGGCGGCGGCGACATGCGCGACAACTGGA
GATCCGAGCTGTATAAGTACAAGGTGGTGAAGATCGAGCCCCTGGGCGTGGCCC
CAACCCGGTGTAAGCGCAGAGTGGTGGGCAGCCACAGCGGCAGCGGCGGCAGC
GGCTCCGGCGGCCACGCCGCCGTGGGCATCGGCGCCGTGTCCCTGGGCTTCCTGG
GCGCCGCCGGCTCCACCATGGGCGCCGCCTCCATGACACTGACAGTGCAGGCCA
GAAATCTGCTGTCCGGCATCGTGCAGCAGCAGTCCAATCTGCTGCGGGCCCCTGA
GCCACAGCAGCACCTGCTGAAGGATACCCACTGGGGCATCAAGCAGCTGCAGGC
CCGGGTGCTGGCCGTGGAGCACTACCTGAGGGATCAGCAGCTGCTGGGCATCTG
GGGCTGTTCCGGCAAGCTGATCTGCTGTACAAACGTGCCCTGGAACAGCTCCTGG
TCCAATAGGAACCTGTCCGAGATCTGGGATAACATGACCTGGCTGCAGTGGGAT
AAGGAGATCAGCAACTACACACAGATCATCTACGGCCTGCTGGAGGAGAGCCAG
AATCAGCAGGAGAAGAACGAGCAGGACCTGCTGGCCCTGGATGGAGGAGGAAG
CGGGGGAAGCGGGGGAAGCGGAGGAAGCGGGGGAAGCGGGGGAAGCAACGCC
GTGGGCCAGGACACCCAGGAAGTGATCGTGGTGCCCCACAGCCTGCCTTTCAAG
GTGGTGGTCATCTCCGCCATCCTGGCCCTGGTCGTGCTGACTATTATTTCCCTGAT
TATCCTGATTATGCTGTGGCAGAAGAAGCCCAGA
TRO11_AY835545_MD39_L14G8-nucleic acid
ATGGATTGGACTTGGATTCTGTTTCTGGTCGCTGCTGCTACTCGGGTGCATTCTCA
GGGCCAGCTGTGGGTCACTGTCTACTACGGCGTGCCAGTGTGGAAGGACGCCTCT
ACCACACTGTTTTGCGCCAGCGACGCCAAGGCCTACGATACAGAGGTGCACAAC
GTGTGGGCAACACACGCATGCGTGCCAACCGATCCAAATCCCCAGGAGGTGGTG
CTGGGCAACGTGACCGAGAACTTCAATATGTGGAAGAACAATATGGTGGACCAG
ATGCACGAGGATATCATCTCTCTGTGGGACCAGAGCCTGAAGCCCTGCGTGAAG
CTGACCCCTCTGTGCGTGACACTGAATTGTACCGATAACATCACCAACACAAATA
CCAACAGCTCCAAGAACTCTAGCACACACTCCTATAACAATTCTCTGGAGGGCGA
GATGAAGAATTGTTCCTTTAACATCACCGCCGGCATCCGGGACAAGGTGAAGAA
GGAGTACGCCCTGTTCTATAAGCTGGATGTGGTGCCCATCGAGGAGGACAAGGA
TACAAATAAGACCACATACCGGCTGCGCAGCTGCAACACATCCGTGATCACCCA
GGCCTGTCCTAAGGTGACCTTTGAGCCTATCCCAATCCACTATTGCGCCCCAGCC
GGCTTCGCCATCCTGAAGTGTAATGACAAGAAGTTTAACGGCACAGGCCCCTGC
ACCAACGTGTCTACAGTGCAGTGTACCCACGGCATCAGGCCTGTGGTGTCCACCC
AGCTGCTGCTGAATGGCTCTCTGGCCGAGGAGGAAGTGATCATCAGAAGCGAGA
ACTTTACAAACAATGCCAAGACCATCATCGTGCAGCTGAATGAGTCTATCGCCAT
CAACTGCACAAGGCCAAACAATAACACCGTGAGAAGCATCCACATCGGACCAGG
AAGGGCCTTCTACTATACCGGCGACATCATCGGCGATATCAGGCAGGCCCACTGT
AATATCTCCAGAACAGAGTGGAACTCTACCCTGCGGCAGATCGTGACAAAGCTG
CGCGAGCAGCTGGGCGACCCTAACAAGACCATCATCTTCGCCCAGTCCTCTGGCG
GCGATACAGAGATCACCATGCACTCCTTTAATTGCGGCGGCGAGTTCTTTTACTG
TAACACCACAAAGCTGTTCAATTCTACCTGGAACGGCAATAACACCACAGAGTC
CGACTCTACAGGCGAGAATATCACCCTGCCATGCCGGATCAAGCAGATCATCAA
CCTGGTGGCAGGAAGTGGGCAAGGCCATGTATGCCCCTCCCATCAAGGGCCAGAT
CTCCTGTAGCTCCAACATCACAGGCCTGCTGCTGACCCGCGACGGCGGAAATAAC
AATTCTAGCGGACCAGAGACATTCAGGCCTGGCGGCGGCAATATGAAGGATAAC
TGGAGAAGCGAGCTGTACAAGTATAAAGTGATCAAGATCGAGCCTCTGGGAGTG
GCACCAACCAGGTGCAAGAGGAGAGTGGTGGGCAGCCACTCCGGCTCTGGCGGC
AGCGGCTCCGGCGGCCACGCAGCAGTGGGCACACTGGGCGCCATGAGCCTGGGC
TTCCTGGGAGCAGCAGGCAGCACCATGGGAGCAGCATCCGTGACACTGACCGTG
CAGGCAAGGCTGCTGCTGTCCGGCATCGTGCAGCAGCAGAACAATCTGCTGAGG
GCACCAGAGCCTCAGCAGCACATGCTGCAGGACACACACTGGGGCATCAAGCAG
CTGCAGGCCCGGGTGCTGGCAGTGGAGCACTACCTGCGCGATCAGCAGCTGCTG
GGCATCTGGGGCTGTAGCGGCAAGCTGATCTGCTGTACCAATGTGCCTTGGAACG
CCTCTTGGAGCAATAAGAGCCTGAACAATATCTGGGAGAATATGACATGGATGA
ACTGGTCCAGAGAGATCGACAACTACACCGATCTGATCTATATCCTGCTGGAGAA
GTCACAGATTCAGCAGGAGAAGAACAATCAGAGCCTGCTGGAACTGGAT
X2278_FJ817366_MD39_L14G8-nucleic acid
ATGGACTGGACCTGGATTCTGTTCCTGGTCGCCGCTGCTACAAGAGTGCAT
TCTACAAATAACCTGTGGGTGACTGTCTACTATGGAGTGCCCGTGTGGAAGGAGG
CCACCACAACCCTGTTCTGCGCCAGCGAGGCCAAGGCCTACGACACAGAGGTGC
ACAACATCTGGGCCACCCACGCCTGCGTGCCTACAGATCCAAACCCCCAGGAGA
TGGAGCTGAAGAATGTGACCGAGAACTTCAACATGTGGAAGAACAATATGGTGG
AGCAGATGCACGAGGACATCATCAGCCTGTGGGATCAGTCCCTGAAGCCCTGCG
TGAAGCTGACACCTCTGTGCGTGACCCTGGATTGTACAAATATCAACAGCACAAA
CTCCACCAACAATACAAGCTCCAATTCTAAGATGGAGGAGACAATCGGCGTGAT
CAAGAATTGTAGCTTCAACGTGACAACCAATATCCGGGACAAGGTGAAGAAGGA
GAACGCCCTGTTTTACTCTCTGGATCTGGTGAGCATCGGCAATTCTAACACCAGC
TATCGCCTGATCTCCTGCAATACCTCTATCATCACACAGGCCTGTCCAAAGGTGA
GCTTCGACCCTATCCCAATCCACTACTGCGCACCAGCAGGATTCGCAATCCTGAA
GTGTAGGGATAAGAAGTTTAACGGCACCGGCCCTTGCAGAAACGTGAGCAGCGT
GCAGTGTACACACGGCATCAGGCCAGTGGTGAGCACCCAGCTGCTGCTGAACGG
CTCCCTGGCAGAGGAGGAGATCATCATCAGATCCGCCAACCTGACCGACAATGC
CAAGACAATCATCATCCAGCTGAACGAGACAATCCAGATCAATTGCACAAGGCC
CAACAATAACACCGTGAGAAGCATCCCAATCGGCCCCGGCCGGACCTTTTACTAT
ACAGGCGACATCATCGGCGATATCCGCAAGGCCTACTGTAACATCTCCGCCACCA
AGTGGAATAACACACTGCGGCAGATCGCCGAGAAGCTGCGCGAGAAGTTCAACA
AGACAATCATCTTTGCCCAGTCCTCTGGCGGCGATCCAGAGGTGGTGAGGCACAC
CTTCAATTGCGGCGGCGAGTTCTTTTACTGTAACAGCTCCCAGCTGTTTAATAGC
ACATGGTATTCCAACGGCACCTCTAATGGCGGCCTGAATAACAGCGCCAACATC
ACCCTGCCCTGCAGAATCAAGCAGATCATCAATCTGTGGCAGGAAGTGGGCAAG
GCCATGTATGCCCCTCCCATCAAGGGCGTGATCAACTGTCTGTCCAATATCACCG
GCATCATCCTGACAAGGGACGGCGGCGAGAATAACGGCACAACCGAGACATTCA
GACCCGGCGGCGGCGACATGAGGGATAACTGGCGCTCTGAGCTGTACAAGTATA
AGGTGGTGAAGATCGAGCCTCTGGGCATCGCCCCAACCAAGTGCAAGAGGAGAG
TGGTGGGCTCTCACAGCGGCTCCGGCGGCTCTGGCAGCGGCGGCCACGCAGCAG
TGGGCCTGGGAGCCGTGTCTCTGGGCTTTCTGGGCCTGGCAGGCTCCACAATGGG
AGCAGCCTCTGTGACACTGACCGTGCAGGCAAGGCTGCTGCTGAGCGGCATCGT
GCAGCAGCAGAATAACCTGCTGAGGGCACCAGAGCCTGCAGCAGCAGCTGCTGCA
GGACACCCACTGGGGCATCAAGCAGCTGCAGGCCCGGGTGCTGGCCCTGGAGCA
CTACCTGAAGGATCAGCAGCTGCTGGGCATCTGGGGCTGTTCCGGCAAGCTGATC
TGCTGTACAACCGTGCCATGGAACGCCTCCTGGTCTAACAAGTCCTATAATCAGA
TCTGGAATAACATGACATGGATGAACTGGAGCAGGGAGATCGACAATTACACCA
ACCTGATCTATAATCTGATTGAAGAGTCACAGTCACAGCAGGAAAAGAACAACC
TGAGCCTGCTGCAGCTGGAC
398F1_HM215312_MD39_L14G8-nucleic acid
ATGGACTGGACTTGGATTCTGTTTCTGGTCGCAGCCGCAACTAGAGTGCAT
AGCATGGGCAACCTGTGGGTCACCGTGTATTACGGGGTGCCAGTGTGGAAGGAC
GCCGAGACTACGCTGTTCTGCGCCTCCGATGCCAAGGCCTACCACACAGAGGTGC
ACAACGTGTGGGCAACCCACGCATGCGTGCCAACAGACCCAAATCCCCAGGAGA
TCAACCTGGAGAATGTGACCGAGGAGTTTAACATGTGGAAGAATAAGATGGTGG
AGCAGATGCACGAGGACATCATCTCCCTGTGGGATCAGTCTCTGAAGCCTTGCGT
GCAGCTGACCCCACTGTGCGTGACACTGGACTGTCAGTACAACGTGACCAACATC
AATAGCACATCCGATATGGCCAGGGAGATCAACAATTGTAGCTATAATATCACC
ACAGAGCTGCGGGATCGCGAGCAGAAAGTGTACAGCCTGTTCTATAGGTCCGAC
ATCGTGCAGATGAACTCCGATAATAGCTCCAAGTACAGACTGATCAACTGCAAT
ACCTCTGCCATCAAGCAGGCCTGTCCAAAGGTGACATTTGAGCCTATCCCAATCC
ACTATTGCGCACCAGCAGGATTCGCAATCCTGAAGTGTAAGGACAAGGAGTTTA
ACGGCACCGGCCCTTGCAAGAACGTGAGCACCGTGCAGTGTACACACGGCATCA
AGCCAGTGGTGAGCACACAGCTGCTGCTGAACGGCTCCCTGGCCGAGGAGAAAG
TGATCATCCGGTCTGAGAATATCACCGATAACGCCAAGAATATCATCGTGCAGCT
GAAGGAGCCCGTGAAGATCAACTGCACCCGGCCTAACAATAACACAGTGAAGTC
CGTGCGCATCGGCCCTGGCCAGACCTTCTACTATACAGGCGAGATCATCGGCGAC
ATCCGCCAGGCCCACTGTAACGTGTCTAAGGCCCACTGGGAGAACACCCTGCAG
GAGGTGGCCAATCAGCTGAAGCTGATGATCCACAGCAACAAGACAATCATCTTC
GCCAATTCTAGCGGCGGCGATCTGGAGATCACCACACACTCTTTTAACTGCGGCG
GCGAGTTCTTTTACTGTTATACCAGCGGCCTGTTCAACTACACCTTCAACGACAC
CAGCACAAACTCCACCGAGTCTAAGAGCAATGATACCATCACACTGCAGTGCAG
GATCAAGCAGATCATCAACATGTGGCAGAGAGCAGGACAGGCCGTGTATGCCCC
TCCCATCCCCGGCATCATCCGGTGTGAGAGCAATATCACCGGCCTGATCCTGACA
CGCGACGGCGGAAATAACAATTCCAACACCAATGAGACATTCAGGCCCGGCGGC
GGCGACATGAGGGATAACTGGAGATCTGAGCTGTACAGATATAAGGTGGTGAAG
ATCGAGCCAATCGGCGTGGCCCCCACCACATGCAAGAGGAGAGTGGTGGGCTCC
CACTCTGGCAGCGGCGGCTCCGGCTCTGGCGGCCACGCAGCCGTGGGCATCGGA
GCCGTGAGCCTGGGCTTTCTGGGAGCAGCAGGCTCTACCATGGGAGCAGCCAGC
ATCACCCTGACAGTGCAGGCAAGGCAGCTGCTGTCCGGAATCGTGCAGCAGCAG
TCTAACCTGCTGAGGGCACCAGAGCCTCAGCAGCACCTGCTGAAGGACACCCAC
TGGGGCATCAAGCAGCTGAAGGCCAGGGTGCTGGCCGTGGAGCACTACCTGAAG
GATCAGCAGCTGCTGGGCATCTGGGGCTGTAGCGGCAAGCTGATCTGCTGTACCA
ACGTGCCCTGGAATTCCTCTTGGTCTAACAAGAGCCTGGGCGAGATCTGGGACAA
CATGACCTGGCTGAATTGGTCCAAGGAGATCGAGAATTACACACAGATCATCTAT
GAGCTGATTGAAGAGTCACAGAACCAGCAGGAGAAAAACAACCAGAGCCTGCT
GGCACTGGAT
246F3_HM215279_MD39_L14G8-nucleic acid
ATGGACTGGACTTGGATTCTGTTTCTGGTCGCAGCCGCTACTCGGGTGAC
TCTATGCAGGACCTGTGGGTGACCGTCTATTATGGGGTGCCAGTGTGGAAGGACG
CCAAGACCACACTGTTCTGCGCCTCCGATGCCAAGGCCTACGAGAAGGAGGTGC
ACAACGTGTGGGCAACCCACGCATGCGTGCCAACAGACCCAAACCCCCAGGAGA
TCGTGATGGCCAATGTGACCGAGGAGTTTAACATGTGGAAGAACAATATGGTGG
AGCAGATGCACGAGGACATCATCTCTCTGTGGGATCAGAGCCTGAAGCCTTGCGT
GAAGCTGACCCCACTGTGCGTGACACTGGACTGTAAGGATTACAACTATTCCATC
ACCAACAATTCTACAGGCATGGAGGGCGAGATCAAGAATTGTTCTTATAACATC
ACCACAGAGCTGCGCGACAAGAGGCAGAAAGTGTACAGCCTGTTCTATCGCCTG
GATGTGGTGCAGATCAATGACTCTAACGATCGCAACAATAGCCAGTACAGGCTG
ATCAATTGCAACACCACAACCATGACCCAGGCCTGTCCTAAGGTGACATTTGACC
CTATCCCAATCCACTATTGCGCCCCAGCCGGCTTCGCCATCCTGAAGTGTAACAA
TAAGACCTTTAATGGCAAGGGCCCCTGCAACAATGTGAGCTCCGTGCAGTGTACC
CACGGCATCAAGCCTGTGGTGTCTACACAGCTGCTGCTGAACGGCAGCCTGGCCG
AGAAGGAGATCATCATCAGGAGCGAGAATCTGACCGACAACGTGAAGACAATCA
TCGTGCACCTGAATGAGAGCGTGGAGATCAACTGCACCAGACCAAACAATAACA
CAGTGAAGTCCGTGCGGATCGGACCAGGACAGACCTTCTACTATACAGGCGATA
TCATCGGCAATATCCGCCAGGCCCACTGTACCGTGAATAAGACAGAGTGGAACA
CAGCCCTGACCAGGGTGAGCAAGAAGCTGAAGGAGTACTTCCCCAACAAGACCA
TCGCCTTTCAGCCTTCTAGCGGCGGCGACCTGGAGATCACAACCTTCTCCTTTAAT
TGCAGAGGCGAGTTCTTTTATTGTAACACATCCGATCTGTTCAATGGCACCTTTA
ACGAGACATCTGGCCAGTTCAATTCCACCTTTAACTCTACACTGCAGTGCCGGAT
CAAGCAGATCATCAATATGTGGCAGGAAGTGGGACAGGCAATGTACGCCCCTCC
CATCGCAGGCAGCATCACCTGTATCTCCAACATCACCGGCCTGATCCTGACACGC
GACGGCGGAAATACAAACTCCACCAAGGAGACATTCAGGCCTGGCGGCGGCAAT
ATGAGAGATAACTGGCGGTCTGAGCTGTACAAGTATAAGGTGGTGAAGATCGAG
CCACTGGGAGTGGCACCAACCAAGTGCAGGAGACGGGTGGTGGGCAGCCACTCC
GGCTCTGGCGGCAGCGGCTCCGGCGGCCACGCAGCAGTGGGCATCGGCGCCGTG
TCTATCGGCTTTCTGGGAGCAGCAGGCTCCACCATGGGAGCAGCCTCTATCACAC
TGACCGTGCAGGCCAGACAGCTGCTGAGCGGCATCGTGCAGCAGCAGTCCAACC
TGCTGAGGGCACCAGAGCCTCAGCAGCACCTGCTGAAGGACACCCACTGGGGCA
TCAAGCAGCTGCAGGCCAGGGTGCTGGCAGTGGAGCACTACCTGAAGGATCAGC
AGCTGCTGGGCATCTGGGGCTGTAGCGGCAAGCTGATCTGCTGTACAAATGTGCC
CTGGAACTCCTCTTGGTCTAACAAGAGCCAGGACGAGATCTGGGATAATATGAC
CTGGCTGAACTGGAGCAAGGAGATCTCCAATTACACACAGATCATCTATAACCTG
ATTGAAGAATCACAGACTCAGCAGGAACTGAATAATAGGTCACTGCTGGCACTG
GAT
CE0217_FJ443575_MD39_L14G8
ATGGACTGGACTTGGATTCTGTTTCTGGTCGCCGCCGCAACTCGCGTGCAT
TCAGCAAAAGATATGTGGGTCACCGTCTATTATGGAGTGCCCGTGTGGCGGGAG
GCCAAGACCACACTGTTTTGCGCAAGCGACGCAAAGGCATACGAGAGGGAGGTG
CACAACGTGTGGGCCACACACGCCTGCGTGCCAACCGATCCAAATCCCCAGGAG
AGAGTGCTGGAGAACGTGACCGAGAATTTCAACATGTGGAAGAACAATATGGTG
GACCAGATGCACGAGGATATCATCTCTCTGTGGGACGAGAGCCTGAAGCCCTGC
ATCAAGCTGACACCTCTGTGCGTGACCCTGAATTGTGGCAACGCCATCGTGAATG
AGTCCACCATCGAGGGCATGAAGAATTGTTCTTTTAACGTGACCACAGAGCTGAA
GGACAAGAAGAAGAAGGAGTACGCCCTGTTCTATAAGCTGGATGTGGTGCCCCT
GAACGGCGAGAACAACAACTCTAACAGCAAGAACTTTAGCGAGTACAGGCTGAT
CAATTGCAACACCTCCACAATCACCCAGGCCTGTCCCAAGGTGTCTTTCGATCCT
ATCCCAATCCACTATTGCGCCCCTGCCGGCTTCGCCATCCTGAAGTGTAATAACG
AGACATTCAACGGCACCGGCCCATGCAATAACGTGTCCACAGTGCAGTGTACCC
ACGGCATCAAGCCCGTGGTGTCTACACAGCTGCTGCTGAATGGCAGCCTGGCCG
AGAAGGAGATCATCATCAGGTCTGAGAACCTGACCAATAACGCCAAGATCATCA
TCGTGCACCTGAATAACCCAGTGAAGATCATCTGCACAAGGCCCGGCAATAACA
CCGTGAAGAGCATGAGAATCGGCCCTGGCCAGACATTCTACTATACCGGCGACA
TCATCGGCGATATCAGGAGAGCCTACTGTAACATCTCTGAGAAGACATGGTATG
ACACCCTGAAGAATGTGAGCGATAAGTTCCAGGAGCACTTTCCTAACGCCTCCAT
CGAGTTCAAGCCATCTGCCGGCGGCGACCTGGAGATCACCACACACTCCTTTAAT
TGCAGGGGCGAGTTCTTTTACTGTGATACAAGCGAGCTGTTCAATGGCACATACA
ATAACTCCACCTATAACAGCTCCAATAACATCACCCTGCAGTGCAAGATCAAGCA
GATCATCAACATGTGGCAGGGCGTGGGCAGAGCCATGTATGCCCCTCCCATCGCC
GGCAATATCACCTGTGAGAGCAACATCACAGGCCTGCTGCTGACCCGGGACGGC
GGAAATAACAAGTCCACACCAGAGACATTCAGGCCCGGCGGCGGCGACATGAGG
GATAACTGGAGAAGCGAGCTGTACAAGTATAAGGTGGTGGAGATCAAGCCTCTG
GGCATCGCCCCAACAAAGTGCAAGAGGAGGGTGGTGGGCTCCCACTCTGGCAGC
GGCGGCTCCGGCTCTGGCGGCCACGCAGCCGTGGGCATGGGCGCCGTGTCTCTG
GGCTTCCTGGGAGCAGCAGGCAGCACCATGGGAGCAGCATCCCTGACACTGACC
GTGCAGGCAAGGCAGCTGCTGAGCGGCATCGTGCAGCAGCAGAATAACCTGCTG
AGAGCCCCCGAGCCTCAGCAGCACATGCTGCAGGACACACACTGGGGCATCAAG
CAGCTGCAGGCCCGGGTGCTGGCAATCGAGCACTACCTGACAGATCAGCAGCTG
CTGGGCATCTGGGGCTGTTCCGGCAAGCTGATCTGCTGTACCAATGTGCCCTGGA
ATAACAGCTGGTCCAACAAGTCCTATGAGGATATCTGGGGCCGGAATATGACCT
GGATGAACTGGAGCAGGGAGATCAACAACTACACAAACACCATCTATCGCCTGC
TGGAAAAGTCACAGAATCAGCAGGAGAAGAATAATAAGTCACTGCTGGAACTGG
AC
CE1176_FJ444437_MD39_L1468-nucleic acid
ATGGATTGGACTTGGATTCTGTTTCTGGTCGCCGCCGCTACTCGCGTGCAT
TCAGTGGGCAACCTGTGGGTCACCGTCTACTATGGGGTGCCCGTGTGGAAGGAG
GCCAAGACCACACTGTTCTGCGCCTCCGACGCCAAGGCCTACGAGAAGGAGGTG
CACAACGTGTGGGCCACACACGCCTGCGTGCCTACCGATCCAAATCCCCAGGAG
ATGGTGCTGGAGAACGTGACAGAGAACTTTAATATGTGGAAGAACGACATGGTG
GATCAGATGCACGAGGACGTGATCTCTCTGTGGGATCAGAGCCTGAAGCCTTGC
GTGAAGCTGACCCCACTGTGCGTGACCCTGACATGTACCAATACCACAGTGTCCA
ACGGCAGCTCCAACTCTAATGCCAACTTCGAGGAGATGAAGAATTGTTCTTTTAA
CGCCACCACAGAGATCAAGGACAAGAAGAAGAACGAGTACGCCCTGTTCTATAA
GCTGGATATCGTGCCCCTGAACAATTCTAGCGGCAAGTATAGGCTGATCAATTGC
AACACAAGCGCCATCGCCCAGGCCTGTCCAAAGGTGACCTTCGAGCCTATCCCA
ATCCACTACTGCGCCCCCGCCGGCTATGCCATCCTGAAGTGTAACAACAAGACCT
TCAACGGCACCGGCCCTTGCAACAACGTGAGCACAGTGCAGTGTACCCACGGCA
TCAAGCCAGTGGTGAGCACCCAGCTGCTGCTGAACGGCTCCCTGGCAGAGAAGG
AGATCATCATCCGGAGCGAGAATCTGACAAACAATGCCAAGACCATCATCATCC
ACCTGAACGAGTCCGTGGGCATCGTGTGCACACGGCCCAGCAACAATACCGTGA
AGTCCATCCGCATCGGCCCTGGCCAGACCTTCTACTATACCGGCGACATCATCGG
CGATATCCGCCAGGCCCACTGTAATGTGAGCAAGCAGAATTGGAACAGGACACT
GCAGCAAGTGGGCAGAAAGCTGGCCGAGCACTTCCCAAATAGGAACATCACCTT
TGCCCACTCCTCTGGCGGCGACCTGGAGATCACCACACACTCCTTCAACTGCAGA
GGCGAGTTCTTTTACTGTAATACATCTGGCCTGTTTAACGGCACCTACCACCCCA
ATGGCACATATAACGAGACAGCCGTGAATAGCTCCGATACAATCACCCTGCAGT
GCAGGATCAAGCAGATCATCAACATGTGGCAGGAAGTGGGCAGAGCCATGTATG
CCCCTCCCATCGCCGGCAATATCACCTGTAACAGCACAATCACCGGCCTGCTGCT
GACACGGGACGGCGGCATCAACCAGACCGGAGAGGAGATCTTCCGCCCCGGCGG
CGGCGACATGCGGGATAATTGGCGCAACGAGCTGTACAAGTATAAGGTGGTGGA
GATCAAGCCACTGGGCATCGCCCCCACAAAGTGCAAGAGGAGAGTGGTGGCTC
CCACTCTGGCAGCGGCGGCTCCGGCTCTGGCGGCCACGCAGCCGTGGGCATCGG
AGCCGTGTCCCTGGGTTTCTGGGAGCAGCAGGCTCTACCATGGGAGCAGCCAG
CATCACACTGACCGTGCAGGCAAGGCAGCTGCTGTCCGGCATCGTGCAGCAGCA
GTCTAACCTGCTGAGAGCCCCCGAGCCTCAGCAGCACATGCTGCAGGACACCCA
CTGGGGCATCAAGCAGCTGCAGGCCAGGGTGCTGGCCATCGAGCACTACCTGAA
GGATCAGCAGCTGCTGGGCATCTGGGGCTGTTCTGGCAAGCTGATCTGCTGTACA
AATGTGCCATGGAACTCTAGCTGGAGCAACCGGTCCCAGGAGGACATCTGGAAC
AATATGACCTGGATGAATTGGAGCAGGGAGATCGATAACTACACACACACCATC
TATAGCCTGCTGGAGGAGTCACAGATTCAGCAGGAGAAAAATAATAAGTCACTG
CTGGCACTGGAC
25710_EF117271_MD39_L146G8-nucleic acid
ATGGACTGGACTTGGATTCTGTTCCTGGTCGCCGCCGCTACTCGCGTGCAT
TCTGGGGGCAACCTGTGGGTCACCGTGTATTATGGAGTGCCCGTGTGGAAGGAG
GCCACCACAACCCTGTTCTGCGCCAGCGACGCCAAGGCCTACGATAAGGAGGTG
CACAACGTGTGGGCAACCCACGCATGCGTGCCAACAGACCCAAACCCCCAGGAG
ATGGTGCTGGGCAATGTGACCGAGAACTTTAATATGTGGAAGAACGAGATGGTG
AATCAGATGCACGAGGACGTGATCTCCCTGTGGGATCAGTCTCTGAAGCCTTGCG
TGAAGCTGACCCCACTGTGCGTGACACTGGAGTGTTCCAACGTGACCTATAATGA
GTCTATGAAGGAGGTGAAGAACTGTTCCTTCAATCTGACAACCGAGCTGAGGGA
TAAGAAGCAGAAGGTGCACGCCCTGTTTTACAGACTGGACATCGTGCCCCTGAA
CGATACCGAGAAGAAGAATAGCTCCCGGCCTTATCGCCTGATCAACTGCAATAC
AAGCGCCATCACCCAGGCCTGTCCTAAGGTGACCTTCGACCCTATCCCAATCCAC
TACTGCACACCAGCCGGCTATGCCATCCTGAAGTGTAACGATAAGAAGTTTAATG
GCACCGGCCCATGCCACAAGGTGTCCACAGTGCAGTGTACCCACGGCATCAAGC
CCGTGGTGTCTACACAGCTGCTGCTGAACGGCAGCCTGGCAGAGGGCGAGATCA
TCATCAGGAGCGAGAACCTGACCAACAATGCCAAGACAATCATCGTGCACCTGA
ATCAGTCCGTGGAGATCGTGTGCGCCCGGCCAAGCAACAATACAGTGACCTCCA
TCAGGATCGGACCAGGACAGACATTCTACTATACCGGCGCCATCACAGGCGACA
TCAGGCAGGCCCACTGTAACATCAGCAAGGATAAGTGGAATGAGACACTGCAGA
GAGTGGGCGAGAAGCTGGCCGAGCACTTCCCCAACAAGACAATCAAGTTTGCCT
CTAGCTCCGGCGGCGACCTGGAGATCACAACCCACTCCTTTAACTGCAGGGGCG
AGTTCTTTTACTGTAATACCTCTGGCCTGTTCAACGGCACCTTTAATGGCACATAC
GTGAGCCCCAACAGCACCGATTCCAATTCTAGCTCCATCATCACAATCCCTTGCC
GGATCAAGCAGATCATCAATATGTGGCAGGAAGTGGGAAGGGCAATGTACGCCC
CTCCCATCGCCGGCAACATCACCTGTAAGTCCAATATCACAGGCCTGCTGCTGGT
GAGGGACGGCGGAACCGGCTCTGAGAGCAACAAGACAGAGATCTTCAGACCCG
GCGGCGGCGACATGAGGGATAATTGGAGATCTGAGCTGTACAAGTATAAGGTGG
TGGAGATCAAGCCACTGGGCGTGGCCCCACCAAGTGCAAGAGGAGAGTGGTGG
GCTCCCACTCTGGCAGCGGCGGCTCCGGCTCTGGCGGCCACGCAGCCGTGGGCAT
CGGAGCCGTGTCCCTGGGCTTTCTGGGAGCAGCAGGCTCTACAATGGGAGCAGC
CAGCATCACACTGACCGTGCAGGCAAGGCAGCTGCTGAGCGGCATCGTGCAGCA
GCAGTCCAACCTGCTGAGGGCACCAGAGCCTCAGCAGCACCTGCTGCAGGACAC
CCACTGGGGCATCAAGCAGCTGCAGACACGGGTGCTGGCCATCGAGCACTACCT
GAAGGATCAGCAGCTGCTGGGCATCTGGGGCTGTTCTGGCAAGCTGATCTGCTGT
ACCGCCGTGCCCTGGAACTATAGCTGGTCCAATCGCAGCCAGGACGATATCTGG
GACAACATGACATGGATGAATTGGTCTAAGGAGATCAGCAACTACACAAATACC
ATCTATAAGCTGCTGGAAGATAGTCAGATTCAGCAGGAAAAGAACAATAAGTCA
CTGCTGGCACTGGAT
BJOX2000_HM215364_MD39_L14G8-nucleic acid
ATGGACTGGACTTGGATTCTGTTTCTGGTCGCAGCAGCAACTCGGGTGCAT
AGCGTCGGCAACCTGTGGGTCACTGTCTACTACGGGGTGCCCGTGTGGAAGGAG
GCCACCACAACCCTGTTCTGCGCCAGCGACGCCAAGGCCTACGATACCGAGGTG
CACAACGTGTGGGCAACCCACGCATGCGTGCCTACAGACCCAGATCCCCAGGAG
ATGTTCCTGGAGAACGTGACAGAGAACTTCAACATGTGGAAGAACAATATGGTG
GACCAGATGCACGAGGATGTGATCAGCCTGTGGGACCAGTCCCTGAAGCCTTGC
GTGAAGCTGACCCCACTGTGCGTGACACTGGAGTGTAAGAATGTGAACAGCTCC
TCTAGCGACACCAAGAACGGCACAGATCCTGAGATGAAGAATTGTTCTTTCAAC
GCCACAACCGAGCTGCGGGACCGCAAGCAGAAGGTGTACGCCCTGTTTTATAAG
CTGGATATCGTGCCACTGAATGAGAAGAACTCCTCTGAGTATCGGCTGATCAAT
GCAACACAAGCACCATCACACAGGCCTGTCCCAAGGTGACCTTCGACCCTATCCC
AATCCACTACTGCACACCTGCCGGCTATGCCATCCTGAAGTGTAATGATGAGAAG
TTTAACGGCACCGGCCCATGCTCCAACGTGAGCACCGTGCAGTGTACACACGGC
ATCAAGCCCGTGGTGAGCACACAGCTGCTGCTGAACGGCTCCCTGGCCGAGAAG
GGCATCATCATCCGCTCCGAGAATCTGACCAACAATGTGAAGACAATCATCGTGC
ACCTGAACCAGTCCGTGGAGATCCTGTGCATCCGGCCAAACAATAACACCGTGA
AGTCTATCCGCATCGGCCCCGGCCAGACCTTCTACTATACAGGCGAGATCATCGG
CGACATCCGGCAGGCCCACTGTAATATCTCTGGCAAGGTCTGGAACGAGACACT
GCAGAGGGTGGGAGAGAAGCTGGCAGAGTACTTCCCAAACAAGACAATCAAGTT
TGCCAGCTCCTCTGGCGGCGATCTGGAGATCACAACCCACTCTTTTAATTGCGGC
GGCGAGTTCTTTTACTGTAACACCAGCAAGCTGTTCAATGGCACCTTTAACGGCA
CATATATGCCTAATGTGACCGAGGGCAACAGCACAATCTCCATCCCATGCCGGAT
CAAGCAGATCATCAATATGTGGCAGAAAGTGGGCCGCGCCATGTATGCCCCTCC
CATCGAGGGCAACATCACCTGTAAGAGCAAGATCACAGGCCTGCTGCTGGAGAG
GGACGGCGGACCAGAGAACGATACCGAGATCTTCAGACCCGGCGGCGGCGACAT
GAGGAATAACTGGAGATCCGAGCTGTACAAGTATAAGGTGGTGGAGATCAAGCC
ACTGGGAGTGGCACCAACCGAGTGCAAGAGGAGAGTGGTGGGCTCTCACAGCGG
CTCCGGCGGCTCTGGCAGCGGCGGCCACGCCGCCGTGGGCATCGGAGCCGTGAG
CCTGGGCTTTCTGGGAGTGGCAGGCTCTACCATGGGAGCAGCAAGCATGGCACT
GACAGTGCAGGCCAGGCAGCTGCTGTCCGGCATCGTGCAGCAGCAGTCTAATCT
GCTGAGAGCACCAGAGCCTCAGCAGCACCTGCTGCAGGACACCCACTGGGGCAT
CAAGCAGCTGCAGACAAGGGTGCTGGCCATCGAGCACTACCTGAAGGATCAGCA
GCTGCTGGGCATCTGGGGCTGTTCCGGCAAGCTGATCTGCTGTACCGCCGTGCCT
TGGAATAGCTCCTGGTCTAACAAGAGCCAGGAGGAGATCTGGGAGAATATGACA
TGGATGAACTGGTCCAAGGAGATCTCTAACTACACCGATACAATCTATAGACTGC
TGGAAGATAGTCAGAATCAGCAGGAGAGAAATAATAAGTCACTGCTGGCACTGG
AT
CH119_EF117261_MD39_L14G8-nucleic acid
ATGGACTGGACTTGGATTCTGTTTCTGGTCGCAGCCGCAACTCGCGTGCAT
TCCGTGGGCAACCTGTGGGTCACCGTCTACTATGGGGTGCCAGTGTGGAAGGAG
GCCACCACAACCCTGTTCTGCGCCTCCGACGCCAAGGCCTACGATACCGAGGTGC
ACAACGTGTGGGCAACACACGCATGCGTGCCAACCGACCCATCTCCCCAGGAGC
TGGTGCTGGAGAATGTGACAGAGAACTTCAACATGTGGAAGAATGAGATGGTGA
ACCAGATGCACGAGGACGTGATCTCCCTGTGGGATCAGTCTCTGAAGCCTTGCGT
GAAGCTGACACCACTGTGCGTGACCCTGGAGTGTTCCAAGGTGTCTAACAATGA
GACAGACAAGTATAACGGCACCGAGGAGATGAAGAATTGTAGCTTCAACGCAAC
AACCGTGGTGCGGGACCGCCAGCAGAAGGTGTACGCCCTGTTTTATAGGCTGGA
TATCGTGCCCCTGACCGAGAAGAATAGCTCCGAGAACTCTAGCAAGTACTATAG
ACTGATCAATTGCAACACATCTGCCATCACCCAGGCCTGTCCAAAGGTGAGCTTC
GAGCCTATCCCAATCCACTACTGCACCCCCGCCGGCTATGCCATCCTGAAGTGTA
ATGACAAGACCTTCAACGGCACCGGCCCTTGCCACAACGTGAGCACAGTGCAGT
GTACCCACGGCATCAAGCCAGTGGTGAGCACACAGCTGCTGCTGAATGGCTCCT
GGCCGAGGGCGAGATCATCATCCGGTCCGAGAACCTGACAAACAATGTGAAGAC
CATCCTGGTGCACCTGAATCAGAGCGTGGAGATCGTGTGCACACGGCCCAACAA
TAACACCGTGAAGTCCATCCGCATCGGCCCTGGCCAGACATTCTACTATACCGGC
GACATCATCGGCGATATCCGGCAGGCCCACTGTAACATCTCCAAGTGGCACGAG
ACACTGAAGCGCGTGTCTGAGAAGCTGGCCGAGCACTTCCCTAATAAGACAATC
AACTTTACCTCCTCTAGCGGCGGCGACCTGGAGATCACAACCCACTCTTTCACCT
GCCGCGGCGAGTTCTTTTACTGTAATACAAGCGGCCTGTTTAACTCCACATACAT
GCCCAATGGCACCTATCTGCACGGCGATACAAATTCCAACTCCTCTATCACCATC
CCTTGCAGGATCAAGCAGATCATCAACATGTGGCAGGAAGTGGGCAGAGCCATG
TATGCCCCTCCCATCGAGGGCAACATCACCTGTAAGTCTAATATCACAGGCCTGC
TGCTGGTGCGGGACGGCGGAACCGAGAGCAATAACACAGAGACAAATAACACA
GAGATCTTCCGCCCCGGCGGCGGCGACATGAGGGATAACTGGAGAAGCGAGCTG
TACAAGTATAAGGTGGTGGAGATCAAGCCACTGGGAGTGGCACCAACCGCATGC
AAGAGGAGAGTGGTGGGCTCTCACAGCGGCTCCGGCGGCTCTGGCAGCGGCGGC
CACGCCGCCGTGGGCATCGGAGCCGTGTCCCTGGGCTTTCTGGGAGTGGCAGGCT
CTACCATGGGAGCAGCCAGCATGACACTGACCGTGCAGGCAAGGCAGCTGCTGT
CCGGCATCGTGCAGCAGCAGTCTAACCTGCTGAGAGCACCAGAGCCTCAGCAGC
ACCTGCTGCAGGACACCCACTGGGGCATCAAGCAGCTGCAGACACGGGTGCTGG
CCATCGAGCACTACCTGAAGGATCAGCAGCTGCTGGGCATCTGGGGCTGTAGCG
GCAAGCTGATCTGCTGTACCGCCGTGCCTTGGAATAGCTCCTGGAGCAACAAGTC
CCAGAAGGAGATCTGGGATAATATGACATGGATGAACTGGTCTAAGGAGATCAG
CAATTACACAAACACCATCTATAAGCTGCTGGAGGACTCACAGAATCAGCAGGA
ATCAAACAACAAATCCCTGCTGGCACTGGAC
X1632_FJ817370_MD39_L14G8-nucleic acid
ATGGACTGGACTTGGATTCTGTTCCTGGTCGCCGCCGCTACACGGGTGCAT
TCATCAAATAACCTGTGGGTCACTGTCTACTATGGGGTGCCCGTGTGGGAGGACG
CCGATACCACACTGTTCTGCGCATCCGACGCAAAGGCATACTCCACCGAGTCTCA
CAACGTGTGGGCAACCCACGCATGCGTGCCAACAGACCCAAACCCCCAGGAGAT
CTATCTGGAGAACGTGACAGAGGACTTCAACATGTGGGAGAACAATATGGTGGA
GCAGATGCAGGAGGACATCATCAGCCTGTGGGATGAGTCCCTGAAGCCTTGCGT
GAAGCTGACCCCACTGTGCGTGACACTGACCTGTACAAATGTGACCAACGTGAC
AGACTCTGTGGGCACAAATAGCCGCCTGAAGGGCTACAAGGAGGAGCTGAAGAA
CTGTAGCTTCAATACCACAACCGAGATCAGGGATAAGAAGAAGCAGGAGTACGC
CCTGTTTTATAAGCTGGACATCGTGCCAATCAATGATAACAGCAACAATTCCAAC
GGCTACAGACTGATCAATTGCAACGTGTCCACCATCAAGCAGGCCTGTCCAAAG
GTGTCTTTCGACCCTATCCCAATCCACTATTGCGCACCAGCAGGATTCGCAATCC
TGAAGTGTCGCGATAAGGAGTTTAATGGCACCGGCACATGCAGGAACGTGAGCA
CCGTGCAGTGTACACACGGCATCAAGCCCGTGGTGTCTACCCAGCTGCTGCTGAA
TGGCAGCCTGGCCGAGGGCGACATCATCATCAGATCCGAGAACATCACCGATAA
TGCCAAGACAATCATCGTGCACCTGAACAAGACCGTGAGCATCACCTGCACACG
CCCCAACAATAACACAGTGAAGTCCATCAGGATCGGCCCTGGCCAGGCCCTGTA
CTATACCGGAGCAATCATCGGCGACACAAGGCAGGCCCACTGTAATATCAACGG
CTCCGAGTGGTACGAGATGATCCAGAATGTGAAGAACAAGCTGAATGAGACATT
CAAGAAGAACATCACATTTGCCCCAGCTCCGGCGGCGATCTGGAGATCACAAC
CCACTCTTTTAACTGCCGCGGCGAGTTCTTTTATTGTAACACCAGCGAGCTGTTCA
ATTCTAGCCACCTGTTTAACGGCTCTACCCTGAGCACAAACGGCACCATCACACT
GCCTTGCAGGATCAAGCAGATCGTGCGCATGTGGCAGAGGGTGGGACAGGCAAT
GTACGCCCCTCCCATCGCCGGCAATATCACCTGTAGATCTAACATCACCGGCCTG
CTGCTGACACGGGACGGCGGAACCAACAAGGATACAAATGAGGCAGAGACATTC
AGACCCGGCGGCGGCGACATGAGAGATAACTGGCGGAGCGAGCTGTACAAGTAT
AAGGTGGTGAAGATCAAGCCACTGGGAGTGGCACCAACCAGGTGCAGGAGACG
GGTGGTGGGCAGCCACTCCGGCTCTGGCGGCAGCGGCTCCGGCGGCCACGCAGC
AATCGGCCTGGGCACCGTGAGCCTGGGCTTTCTGGGAACCGCAGGCTCCACAAT
GGGAGCAGCCTCTATCACCCTGACAGTGCAGGTGAGACAGCTGCTGAGCGGCAT
CGTGCAGCAGCAGTCCAACCTGCTGAGGGCACCAGAGCCTCAGCAGCACCTGCT
GCAGGACACCCACTGGGGCATCAAGCAGCTGCAGGCCCGCGTGCTGGCAGTGGA
GCACTACCTGAAGGATCAGCAGATCCTGGGCATCTGGGGCTGTTCCGGCAAGCT
GATCTGCTGTACCAACGTGCCCTGGAATTCCTCTTGGTCTAATAAGTCTTATAGC
GACATCTGGGATAACCTGACATGGATCAATTGGTCCAGGGAGATCTCTAACTACA
CCCAGCAGATCTATACACTGCTGGAAGAAAGTCAGAATCAGCAGGAGAAGAATA
ATCAGAGCCTGCTGGCACTGGAT
CNE8_HM215427_MD39_L14G8-nucleic acid
ATGGACTGGACTTGGATTCTGTTCCTGGTCGCCGCTGCTACACGAGTGCAT
TCATCTGATAACCTGTGGGTCACCGTCTACTATGGCGTGCCAGTGTGGCGGGACG
CCGATACCACACTGTTCTGCGCCAGCGACGCCAAGGCCTACGATACCGAGGTGC
ACAACGTGTGGGCAACCCACGCATGCGTGCCAACAGACCCTAATCCACAGGAGA
TCCACCTGGAGAACGTGACAGAGAACTTCAACATGTGGAAGAACAAGATGGCCG
AGCAGATGCAGGAGGACGTGATCTCCCTGTGGGATGAGTCTCTGAAGCCCTGCG
TGCAGCTGACCCCTCTGTGCGTGACACTGAATTGTACCAATGCCAACCTGAATGC
CACCGTGAATGCCTCCACCACAATCGGCAACATCACAGATGAGGTGCGGAACTG
TTCTTTCAATACCACAACCGAGCTGCGCGACAAGAAGCAGAACGTGTACGCCCT
GTTTTATAAGCTGGATATCGTGCCCATCAACAATAACTCCGAGTATCGGCTGATC
AACTGCAATACCTCTGTGATCAAGCAGGCCTGTCCTAAGGTGAGCTTCGACCCCA
TCCCTATCCACTACTGCGCACCAGCAGGATATGCAATCCTGCGCTGTAATGATAA
GAACTTTAATGGCACAGGCCCCTGCAAGAACGTGAGCTCCGTGCAGTGTACCCA
CGGCATCAAGCCTGTGGTGTCTACACAGCTGCTGCTGAACGGCAGCCTGGCCGA
GGACGAGATCATCATCAGGAGCGAGAACCTGACAGATAATGTGAAGACCATCAT
CGTGCACCTGAACAAGTCCGTGGAGATCAATTGCACCAGGCCATCTAATAACAC
AGTGACCAGCGTGAGAATCGGCCCCGGCCAGGTGTTCTACTATACAGGCGACAT
CATCGGCGATATCCGGAAGGCCTACTGTGAGATCAATCGCACAAAGTGGCACGA
GACACTGAAGCAGGTGGCCACCAAGCTGAGGGAGCACTTCAACAAGACAATCAT
CTTTCAGCCCCCTTCCGGCGGCGACATCGAGATCACCATGCACCACTTCAACTGC
AGAGGCGAGTTCTTTTACTGTAACACAACCAAGCTGTTTAATTCTACCTGGGGCG
AGAACACAACCATGGAGGGCCACAATGATACAATCGTGCTGCCTTGCAGAATCA
AGCAGATCGTGAACATGTGGCAGGGAGTGGGACAGGCAATGTATGCCCCACCCA
TCAGGGGCAGCATCAACTGCGTGAGCAATATCACAGGCATCCTGCTGACCAGAG
ACGGCGGAACAAACATGTCTAATGAGACATTCAGGCCTGGCGGCGGCAACATCA
AGGATAATTGGAGAAGCGAGCTGTACAAGTATAAGGTGGTGGAGATCGAGCCTC
TGGGCATCGCCCCAACAAAGTGCAAGAGGAGAGTGGTGGGCTCTCACAGCGGCT
CCGGCGGCTCTGGCAGCGGCGGCCACGCCGCCGTGGGCATCGGCGCCATGAGCT
TCGGCTTTCTGGGAGCAGCAGGCTCCACCATGGGAGCAGCCTCTATCACACTGAC
CGTGCAGGCAAGGCAGCTGCTGAGCGGCATCGTGCAGCAGCAGTCCAACCTGCT
GAGGGCACCAGAGCCACAGCAGCACCTGCTGCAGGACACCCACTGGGGCATCAA
GCAGCTGCAGGCCCGCGTGCTGGCAGTGGAGCACTACCTGAAGGATCAGAAGTT
TCTGGGCCTGTGGGGCTGTTCCGGCAAGATCATCTGCTGTACCGCCGTGCCTTGG
AACTCCACATGGTCTAATCGGAGCTATGAGGAGATCTGGGACAACATGACCTGG
ATCAATTGGTCCCGCGAGATCTCTAACTACACAAGCCAGATCTATGAGATCCTGA
CCGAATCACAGAATCAGCAGGACAGAAACAACAAATCACTGCTGGAACTGGAC
CNE55_HM215418_MD39_L14G8-nucleic acid
ATGGACTGGACTTGGATTCTGTTCCTGGTCGCTGCCGCTACACGAGTGCATTCCT
CTGATAAACTGTGGGTGACCGTCTACTATGGAGTGCCAGTGTGGCGGGACGCCG
ATACCACACTGTTCTGCGCCTCTGACGCCAAGGCCCACGAGACAGAGGTGCACA
ACGTGTGGGCAACCCACGCATGCGTGCCAACAGATCCTAACCCACAGGAGATCC
ACCTGGTGAATGTGACAGAGAACTTTAATATGTGGAAGAACAAGATGGTGGAGC
AGATGCAGGAGGACGTGATCAGCCTGTGGGATGAGTCCCTGAAGCCCTGCGTGA
AGCTGACCCCTCTGTGCGTGACACTGAACTGTACCACAGCCAACACCAATGAGA
CAAAGAACAATACCACAGACGATAATATCAAGGACGAGATGAAGAACTGTACCT
TCAATATGACCACAGAGATCCGGGACAAGAAGCAGCGCGTGAGCGCCCTGTTTT
ACAAGCTGGATATCGTGCCCATCGACGATAGCAAGAACAATTCCGAGTATCGCC
TGATCAACTGCAATACCAGCGTGATCAAGCAGGCCTGTCCTAAGGTGTCCTTCGA
CCCCATCCCTATCCACTACTGCACCCCAGCCGGCTATGTGATCCTGAAGTGTAAC
GATAAGAACTTTAATGGCACAGGCCCCTGCAAGAATGTGAGCTCCGTGCAGTG
ACCCACGGCATCAAGCCTGTGGTGTCCACACAGCTGCTGCTGAACGGCTCTCTGG
CCGAGGAGGAGATCATCATCAGGTCTGAGAATCTGACCGATAACGCCAAGAATA
TCATCGTGCACCTGAACAAGAGCGTGGAGATCAATTGCACACGGCCATCTAACA
ATACCGTGACAAGCGTGCGCATCGGACCAGGACAGGTGTTCTACTATACCGGCG
ACATCACAGGCGATATCAGAAAGGCCTACTGTGAGATCGACGGCACCGAGTGGA
ACAAGACCCTGACACAGGTGGCCGAGAAGCTGAAGGAGCACTTTAATAAGACCA
TCGTGTACCAGCCCCTTCCGGCGGCGATCTGGAGATCACAATGCACCACTTCAA
CTGCCGGGGCGAGTTCTTTTATTGTAATACCACACAGCTGTTTAACAATTCTGTG
GGCAACAGCACCATCAAGCTGCCTTGCCGCATCAAGCAGATCATCAATATGTGG
CAGGGAGTGGGACAGGCAATGTACGCCCCACCCATCAGCGGAGCCATCAACTGT
CTGTCCAATATCACCGGCATCCTGCTGACAAGGGACGGCGGCGGAAACAATAGG
TCCAATGAGACATTCAGGCCTGGCGGCGGCAACATCAAGGATAATTGGAGATCT
GAGCTGTACAAGTATAAGGTGGTGGAGATCGAGCCTCTGGGCATCGCCCCAACA
AAGTGCAAGAGGAGAGTGGTGGGCTCTCACAGCGGCTCCGGCGGCTCTGGCAGC
GGCGGCCACGCCGCCGTGGGCATCGGCGCCATGAGCTTCGGCTTTCTGGGAGCA
GCAGGCTCCACCATGGGAGCAGCCTCTATCACCCTGACAGTGCAGGCCCGGCAG
CTGCTGTCTGGCATCGTGCAGCAGCAGAGCAACCTGCTGAGGGCACCAGAGCCA
CAGCAGCACATGCTGCAGGACACACACTGGGGCATCAAGCAGCTGCAGGCCAGG
GTGCTGGCAGTGGAGCACTACCTGAAGGATCAGAGATTTCTGGGCCTGTGGGGC
TGTAGCGGCAAGACCATCTGCTGTACAGCCGTGCCTTGGAACTCCACCTGGTCTA
ATAAGACATATGAGGAGATCTGGGACAACATGACCTGGACAAATTGGTCCCGGG
AGATCTCTAACTACACCAATCAGATCTATTCCATTCTGACCGAATCACAGTCACA
GCAGGATAAAAATAACAAAAGTCTGCTGGAACTGGAT
AD8_MD64_link14_TS1-nucleic acid
GGATCCGCCACCATGGACTGGACTTGGATTCTGTTCCTGGTCGCCGCCGCT
ACTCGGGTGCATTCTGTCGAAAACCTGTGGGTGACTGTCTATTATGGAGTGCCCG
TGTGGAAGGAGGCCACCACAACCCTGTTCTGCGCCTCCGACGCCAAGGCCTACG
ATACCGAGGTGCACAACGTGTGGGCCACCCACGAGTGCGTGCCTACAGACCCAA
ACCCCCAGGAGGTGGTGCTGGAGAATGTGACAGAGAACTTCAACATGTGGAAGA
ACAATATGGTGGAGCAGATGCACGAGGACATCATCGAGCTGTGGGATCAGAGCC
TGAAGCCTTGCGTGAAGCTGACCCCACTGTGCGTGACCCTGAATTGTACAGACCT
GCGGAATGTGACAAACATCAACAATAGCTCCGAGGGCATGAGAGGCGAGATCAA
GAATTGTAGCTTCAACATCACAACCTCCATCAGGGACAAGGTGAAGAAGGATTA
CGCCCTGTTTTATCGCCTGGATGTGGTGCCCATCGACAATGATAACACCTCTTAC
CGGCTGATCAATTGCAACACAAGCACCATCACACAGGCCTGTCCAAAGGTGTCCT
TCGAGCCTATCCCAATCCACTATTGCACCCCCGCCGGCTTCGCCATCCTGAAGTG
TAAGGACAAGAAGTTTAACGGCACAGGCCCTTGCAAGAACGTGAGCACCGTGCA
GTGTACACACGGCATCCGGCCAGTGGTGAGCACCCAGCTGCTGCTGAACGGCTC
CCTGGCAGAGGAGGAAGTGATCATCAGATCTAGCAATTTCACAGATAATGCCAA
GAACATCATCGTGCAGCTGAAGGAGTCCGTGGAGATCAACTGCACCCGGCCCAA
CAATAACACAGTGAAGTCTATCCACATCGGCCCTGGCAGAGCCTTTTACTATACC
GGCGACATCATCGGCGATATCAGGCAGGCCCACTGTAACATCAGCCGCACCAAG
TGGAATAACACACTGAATCAGATCGCCACCAAGCTGAAGGAGCAGTTCGGCAAT
AACAAGACAATCGTGTTTAACCAGTCCTCTGGCGGCGACCCAGAGATCGTGATG
CACTCTTTTAATTGCGGCGGCGAGTTCTTTTACTGTAACTCTACCCAGCTGTTCAA
TAGCACATGGAACTTCAACGGCACCTGGAATCTGACACAGAGCAACGGCACCGA
GGGCAATGATACCATCACACTGCCCTGCAGGATCAAGCAGATCATCAACATGTG
GCAGGAAGTGGGCAAGGCCATGTATGCCCCTCCCATCAGGGGCCAGATCCGCTG
TAGCTCCAATATCACCGGCCTGATCCTGACAAGGGACGGCGGAAATAACCACAA
TAACGATACCGAGACATTCCGCCCCGGCGGCGGCGACATGAGGGATAACTGGAG
ATCCGAGCTGTACAAGTATAAGGTGGTGAAGATCGAGCCACTGGGAGTGGCACC
AACCAAGTGCAAGAGGAGAGTGGTGCAGTCTCACAGCGGCTCCGGCGGCTCTGG
CAGCGGCGGCCACGCCGCCGTGGGCACCATCGGCGCCATGAGCCTGGGCTTTCT
GGGAGCAGCAGGCTCCACAATGGGAGCAGCCTCTATCACCCTGACAGTGCAGGC
CAGGCTGCTGCTGTCCGGCATCGTGCAGCAGCAGAATAACCTGCTGAGGGCACC
AGAGCCTCAGCAGCACCTGCTGCAGCTGACCGTGTGGGGCATCAAGCAGCTGCA
GGCCCGGGTGCTGGCAGTGGAGCACTATCTGAGAGATCAGCAGCTGCTGGGAAT
CTGGGGATGCAGCGGCAAGCTGATCTGCTGTACCGCCGTGCCATGGAACGCCTCC
TGGTCTAATAAGACCCTGGACATGATCTGGAATAACATGACATGGATGGAGTGG
GAGCGCGAGATCGATAACTACACCGGCCTGATCTATACACTGATCGAGGAATCA
CAGAATCAGCAGGAGAAAAACGAACAGGAACTGCTGGAACTGGATGGCGGCGT
CGAAAATCTCTGGGTCACCGTCTATTATGGGGTCCCTGTCTGGAAGGAAGCAACT
ACTACTCTGTTCTGTGCCTCCGATGCCAAGGCCTACGACACAGAGGTGCACAACG
TGTGGGCTACACACGAGTGCGTGCCAACCGATCCAAACCCCCAGGAGGTGGTGC
TGGAGAACGTGACCGAGAACTTCAACATGTGGAAGAACAACATGGTGGAGCAGA
TGCACGAGGACATCATCGAGCTGTGGGATCAGTCCCTGAAGCCTTGCGTGAAGCT
GACACCACTGTGCGTGACACTGAACTGTACCGACCTGAGGAACGTGACCAACAT
CAACAACAGCTCCGAGGGAATGAGAGGCGAGATCAAGAACTGTAGCTTCAACAT
CACCACATCCATCCGGGACAAGGTGAAGAAGGATTACGCCCTGTTTTACCGCCTG
GATGTGGTGCCCATCGACAACGATAACACCTCTTACAGGCTGATCAACTGCAACA
CCAGCACAATCACCCAGGCTTGTCCAAAGGTGTCCTTTGAGCCTATCCCAATCCA
CTACTGCACACCCGCCGGCTTCGCTATCCTGAAGTGTAAGGACAAGAAGTTTAAC
GGAACCGGCCCTTGCAAGAACGTGTCTACAGTGCAGTGTACCCACGGCATCAGG
CCAGTGGTGAGCACACAGCTGCTGCTGAACGGCAGCCTGGCCGAGGAGGAAGTG
ATCATCAGATCTAGCAACTTCACCGATAACGCTAAGAACATCATCGTGCAGCTGA
AGGAGTCCGTGGAGATCAACTGCACAAGGCCCAACAACAACACCGTGAAGTCTA
TCCACATCGGACCTGGCAGAGCCTTTTACTACACAGGAGACATCATCGGCGATAT
CCGGCAGGCTCACTGTAACATCAGCCGCACAAAGTGGAACAACACCCTGAACCA
GATCGCCACAAAGCTGAAGGAGCAGTTCGGCAACAACAAGACCATCGTGTTTAA
CCAGTCCAGCGGCGGCGACCCCGAGATCGTGATGCACTCTTTCAACTGCGGCGG
AGAGTTCTTTTACTGTAACTCTACACAGCTGTTCAACAGCACCTGGAACTTTAAC
GGAACATGGAACCTGACCCAGAGCAACGGAACCGAGGGCAACGATACAATCAC
CCTGCCTTGCCGGATCAAGCAGATCATCAACATGTGGCAGGAAGTGGGAAAGGC
CATGTACGCTCCCCCTATCAGGGGACAGATCAGGTGTAGCTCCAACATCACAGG
ACTGATCCTGACCCGGGACGGCGGAAACAACCACAACAACGATACAGAGACATT
CAGGCCTGGCGGAGGCGACATGAGGGATAACTGGAGATCCGAGCTGTACAAGTA
CAAGGTGGTGAAGATCGAGCCACTGGGAGTGGCTCCAACCAAGTGCAAGAGGAG
AGTGGTGCAGTCTCACAGCGGCAGCGGCGGCAGCGGCAGCGGAGGCCACGCTGC
TGTGGGAACAATCGGAGCTATGAGCCTGGGATTTCTGGGAGCTGCTGGCAGCAC
CATGGGAGCTGCTTCTATCACACTGACCGTGCAGGCTAGGCTGCTGCTGTCCGGA
ATCGTGCAGCAGCAGAACAACCTGCTGAGGGCTCCAGAGCCTCAGCAGCACCTG
CTGCAGCTGACAGTGTGGGGCATCAAGCAGCTGCAGGCCAGGGTGCTGGCTGTG
GAGCACTACCTGAGGGACCAGCAGCTGCTGGGCATCTGGGGATGTAGCGGCAAG
CTGATCTGCTGTACCGCCGTGCCATGGAACGCTTCCTGGTCTAACAAGACACTGG
ACATGATCTGGAACAACATGACCTGGATGGAGTGGGAGCGCGAGATCGATAACT
ACACAGGCCTGATCTACACCCTGATCGAAGAAAGTCAGAATCAGCAGGAAAAGA
ACGAACAGGAACTGCTGGAACTGGACGGTGGCGTCGAGAATCTGTGGGTCACCG
TCTATTATGGAGTCCCCGTCTGGAAAGAGGCTACTACTACACTGTTTTGTGCAAG
CGATGCCAAGGCCTACGACACAGAGGTGCACAACGTGTGGGCCACACACGAGTG
CGTGCCAACCGATCCAAACCCCCAGGAGGTGGTGCTGGAGAATGTGACCGAGAA
TTTCAACATGTGGAAGAACAATATGGTGGAGCAGATGCACGAGGACATCATCGA
GCTGTGGGATCAGTCCCTGAAGCCTTGCGTGAAGCTGACACCACTGTGCGTGACA
CTGAACTGTACCGACCTGAGGAATGTGACCAACATCAACAATAGCTCCGAGGGC
ATGAGAGGCGAGATCAAGAATTGTAGCTTCAACATCACCACATCCATCCGGGAC
AAGGTGAAGAAGGATTACGCCCTGTTTTATCGCCTGGATGTGGTGCCCATCGACA
ATGATAACACCTCTTACAGGCTGATCAATTGCAACACCAGCACAATCACCCAGGC
CTGTCCAAAGGTGTCCTTTGAGCCTATCCCAATCCACTATTGCACACCCGCCGGC
TTCGCCATCCTGAAGTGTAAGGACAAGAAGTTTAACGGCACCGGCCCTTGCAAG
AACGTGAGCACAGTGCAGTGTACCCACGGCATCAGGCCAGTGGTGAGCACACAG
CTGCTGCTGAACGGCTCCCTGGCCGAGGAGGAAGTGATCATCAGATCTAGCAATT
TCACCGATAATGCCAAGAACATCATCGTGCAGCTGAAGGAGTCCGTGGAGATCA
ACTGCACAAGGCCCAACAATAACACCGTGAAGTCTATCCACATCGGCCCTGGCA
GAGCCTTTTACTATACCGGCGACATCATCGGCGATATCCGGCAGGCCCACTGTAA
CATCAGCCGCACAAAGTGGAATAACACCCTGAATCAGATCGCCACAAAGCTGAA
GGAGCAGTTCGGCAATAACAAGACCATCGTGTTTAACCAGTCCTCTGGCGGCGA
CCCCGAGATCGTGATGCACTCTTTCAATTGCGGCGGCGAGTTCTTTTACTGTAACT
CTACACAGCTGTTCAATAGCACCTGGAACTTCAACGGCACATGGAATCTGACCCA
GAGCAACGGCACCGAGGGCAATGATACAATCACCCTGCCTTGCCGGATCAAGCA
GATCATCAACATGTGGCAGGAAGTGGGCAAGGCCATGTATGCCCCTCCCATCAG
GGGACAGATCAGGTGTAGCTCCAATATCACAGGCCTGATCCTGACCCGGGACGG
CGGAAATAACCACAATAACGATACAGAGACATTCAGGCCCGGCGGCGGCGACAT
GAGGGATAACTGGAGATCCGAGCTGTACAAGTATAAGGTGGTGAAGATCGAGCC
ACTGGGAGTGGCACCAACCAAGTGCAAGAGGAGAGTGGTGCAGTCTCACAGCGG
CTCCGGCGGCTCTGGCAGCGGCGGCCACGCAGCAGTGGGAACAATCGGAGCAAT
GAGCCTGGGCTTTCTGGGAGCAGCAGGCTCCACCATGGGAGCAGCCTCTATCAC
ACTGACCGTGCAGGCAAGGCTGCTGCTGTCCGGCATCGTGCAGCAGCAGAATAA
CCTGCTGAGGGCACCAGAGCCTCAGCAGCACCTGCTGCAGCTGACAGTGTGGGG
CATCAAGCAGCTGCAGGCCAGGGTGCTGGCAGTGGAGCACTATCTGAGGGACCA
GCAGCTGCTGGGCATCTGGGGCTGTAGCGGCAAGCTGATCTGCTGTACCGCCGTG
CCCTGGAACGCCTCCTGGTCTAATAAGACACTGGACATGATCTGGAATAACATGA
CCTGGATGGAGTGGGAGCGCGAGATCGATAACTACACAGGCCTGATCTATACCC
TGATTGAGGAGTCACAGAACCAGCAGGAAAAGAACGAACAGGAACTGCTGGAA
CTGGATTGATAACTCGAG
AD8_MD64_link14-nucleic acid
GGATCCGCCACCATGGACTGGACTTGGATTCTGTTCCTGGTCGCCGCCGCT
ACTCGGGTGCATTCTGTCGAAAACCTGTGGGTGACTGTCTATTATGGAGTGCCCG
TGTGGAAGGAGGCCACCACAACCCTGTTCTGCGCCTCCGACGCCAAGGCCTACG
ATACCGAGGTGCACAACGTGTGGGCCACCCACGAGTGCGTGCCTACAGACCCAA
ACCCCCAGGAGGTGGTGCTGGAGAATGTGACAGAGAACTTCAACATGTGGAAGA
ACAATATGGTGGAGCAGATGCACGAGGACATCATCGAGCTGTGGGATCAGAGCC
TGAAGCCTTGCGTGAAGCTGACCCCACTGTGCGTGACCCTGAATTGTACAGACCT
GCGGAATGTGACAAACATCAACAATAGCTCCGAGGGCATGAGAGGCGAGATCAA
GAATTGTAGCTTCAACATCACAACCTCCATCAGGGACAAGGTGAAGAAGGATTA
CGCCCTGTTTTATCGCCTGGATGTGGTGCCCATCGACAATGATAACACCTCTTAC
CGGCTGATCAATTGCAACACAAGCACCATCACACAGGCCTGTCCAAAGGTGTCCT
TCGAGCCTATCCCAATCCACTATTGCACCCCCGCCGGCTTCGCCATCCTGAAGTG
TAAGGACAAGAAGTTTAACGGCACAGGCCCTTGCAAGAACGTGAGCACCGTGCA
GTGTACACACGGCATCCGGCCAGTGGTGAGCACCCAGCTGCTGCTGAACGGCTC
CCTGGCAGAGGAGGAAGTGATCATCAGATCTAGCAATTTCACAGATAATGCCAA
GAACATCATCGTGCAGCTGAAGGAGTCCGTGGAGATCAACTGCACCCGGCCCAA
CAATAACACAGTGAAGTCTATCCACATCGGCCCTGGCAGAGCCTTTTACTATACC
GGCGACATCATCGGCGATATCAGGCAGGCCCACTGTAACATCAGCCGCACCAAG
TGGAATAACACACTGAATCAGATCGCCACCAAGCTGAAGGAGCAGTTCGGCAAT
AACAAGACAATCGTGTTTAACCAGTCCTCTGGCGGCGACCCAGAGATCGTGATG
CACTCTTTTAATTGCGGCGGCGAGTTCTTTTACTGTAACTCTACCCAGCTGTTCAA
TAGCACATGGAACTTCAACGGCACCTGGAATCTGACACAGAGCAACGGCACCGA
GGGCAATGATACCATCACACTGCCCTGCAGGATCAAGCAGATCATCAACATGTG
GCAGGAAGTGGGCAAGGCCATGTATGCCCCTCCCATCAGGGGCCAGATCCGCTG
TAGCTCCAATATCACCGGCCTGATCCTGACAAGGGACGGCGGAAATAACCACAA
TAACGATACCGAGACATTCCGCCCCGGCGGCGGCGACATGAGGGATAACTGGAG
ATCCGAGCTGTACAAGTATAAGGTGGTGAAGATCGAGCCACTGGGAGTGGCACC
AACCAAGTGCAAGAGGAGAGTGGTGCAGTCTCACAGCGGCTCCGGCGGCTCTGG
CAGCGGCGGCCACGCCGCCGTGGGCACCATCGGCGCCATGAGCCTGGGCTTTCT
GGGAGCAGCAGGCTCCACAATGGGAGCAGCCTCTATCACCCTGACAGTGCAGGC
CAGGCTGCTGCTGTCCGGCATCGTGCAGCAGCAGAATAACCTGCTGAGGGCACC
AGAGCCTCAGCAGCACCTGCTGCAGCTGACCGTGTGGGGCATCAAGCAGCTGCA
GGCCCGGGTGCTGGCAGTGGAGCACTATCTGAGAGATCAGCAGCTGCTGGGAAT
CTGGGGATGCAGCGGCAAGCTGATCTGCTGTACCGCCGTGCCATGGAACGCCTCC
TGGTCTAATAAGACCCTGGACATGATCTGGAATAACATGACATGGATGGAGTGG
GAGCGCGAGATCGATAACTACACCGGCCTGATCTATACACTGATCGAGGAATCA
CAGAATCAGCAGGAGAAAAACGAACAGGAACTGCTGGAACTGGATTGATAACTC
GAG
001428_MD39_link14_TS1-nucleic acid
GGATCCGCCACCATGGACTGGACTTGGATTCTGTTCCTGGTGGCAGCAGC
AACTAGAGTGCATTCCGTCGAAAACCTGTGGGTGACCGTGTATTATGGAGTGCCC
GTGTGGAAGGAGGCCCGGACCACACTGTTCTGCGCCTCCGACGCCAAGGCCTAC
GAGACAGAGGTGCACAACGTGTGGGCCACACACGCCTGCGTGCCTACCGATCCA
AATCCCCAGGAGATGGTGCTGGGCAACGTGACCGAGAACTTTAATATGTGGAAG
AACGACATGGTGGATCAGATGCACGAGGACGTGATCTCTCTGTGGGCCCAGAGC
CTGAAGCCTTGCGTGAAGCTGACCCCACTGTGCGTGACACTGGAGTGTACCCAGG
TGAACGCCACACAGGGCAATACCACACAGGTGAACGTGACCCAAGTGAATGGCG
ACGAGATGAAGAACTGTTCCTTCAATACCACAACCGAGATCCGGGATAAGAAGC
AGAAGGCCTACGCCCTGTTTTATAGACTGGACCTGGTGCCTCTGGAGCGGGAGA
ACAGAGGCGATTCTAATAGCGCCTCCAAGTATATCCTGATCAACTGCAATACATC
TGCCATCACCCAGGCCTGTCCTAAAGTGAATTTCGATCCTATCCCAATCCACTAC
TGCACCCCAGCCGGCTATGCCATCCTGAAGTGTAACAACAAGACCTTCAACGGC
ACCGGCTCCTGCAACAACGTGAGCACAGTGCAGTGTACCCACGGCATCAAGCCA
GTGGTGAGCACCCAGCTGCTGCTGAACGGCTCCCTGGCAGAGGAGGAGATCATC
ATCAGGTCCGAGAACCTGACAGACAATGTGAAGACCATCATCGTGCACCTGGAT
CAGTCCGTGGAGATCGTGTGCACACGGCCAAACAATAACACCGTGAAGTCTATC
AGAATCGGCCCCGGCCAGACATTCTACTATACCGGCGACATCATCGGCAATATCC
GGGAGGCCCACTGTAACATCTCTGAGAAGAAGTGGCACGAGATGCTGCGGAGAG
TGAGCGAGAAGCTGGCCGAGCACTTCCCCAATAAGACAATCAAGTTTACCAGCT
CCTCTGGCGGCGATCTGGAGATCACAACCCACAGCTTCAACTGCAGAGGCGAGT
TCTTTTACTGTAACACCAGCGGCCTGTTTAATTCCACATACATGCCCAACGGCAC
CTATATGCCTAATGGCACAAATAACTCTAACAGCACCATCATCCTGCCATGCCGG
ATCAAGCAGATCATCAATATGTGGCAGGAAGTGGGCAGAGCCATGTATGCCCCT
CCCATCGCCGGCAACATCACATGTAACAGCAATATCACCGGCCTGCTGCTGGTGA
GGGACGGCGGCAAGAATAACAATACAGAGATCTTCCGCCCCGGCGGCGGCGACA
TGAGGGATAACTGGCGCTCCGAGCTGTACAAGTATAAGGTGGTGGAGATCAAGC
CACTGGGAGTGGCACCAACCAGGTGCAAGAGGCGCGTGGTGGGCTCCCACTCTG
GCAGCGGCGGCTCCGGCTCTGGCGGCCACGCAGCAGTGGGCCTGGGAGCCGTGA
GCCTGGGCTTTCTGGGAGCAGCAGGCTCTACCATGGGAGCAGCCAGCATCACAC
TGACCGTGCAGGCAAGGCAGCTGCTGTCCGGCATCGTGCAGCAGCAGTCTAACC
TGCTGCAGGCACCAGAGCCTCAGCAGCACCTGCTGCAGGACACACACTGGGGCA
TCAAGCAGCTGCAGACCCGCGTGCTGGCCATCGAGCACTACCTGAAGGATCAGC
AGCTGCTGGGCATCTGGGGCTGCTCTGGCAAGCTGATCTGCTGTACAGCCGTGCC
TTGGAACAGCTCCTGGAGCAATAAGTCCCTGACAGACATCTGGGATAATATGAC
CTGGATGCAGTGGGATAGGGAGGTGAGCAACTACACCGGCATCATCTATCGCCT
GCTGGAAGACTCACAGAATCAGCAGGAAAGGAATGAACAGGATCTGCTGGCACT
GGACGGGGGAGTCGAGAACCTCTGGGTCACCGTGTATTATGGAGTCCCCGTCTG
GAAAGAAGCCCGAACCACCCTGTTTTGTGCCTCTGATGCTAAAGCCTACGAGACA
GAGGTGCACAACGTGTGGGCTACACACGCTTGCGTGCCAACCGACCCAAACCCC
CAGGAGATGGTGCTGGGCAACGTGACCGAGAACTTCAACATGTGGAAGAACGAC
ATGGTGGATCAGATGCACGAGGATGTGATCTCTCTGTGGGCCCAGAGCCTGAAG
CCTTGCGTGAAGCTGACCCCACTGTGCGTGACACTGGAGTGTACCCAGGTGAACG
CTACACAGGGCAACACCACACAGGTGAACGTGACCCAGGTGAACGGAGACGAG
ATGAAGAACTGTTCCTTCAACACCACAACCGAGATCAGGGATAAGAAGCAGAAG
GCCTACGCTCTGTTTTACAGACTGGACCTGGTGCCACTGGAGAGGGAGAACAGA
GGCGATTCTAACAGCGCCTCCAAGTACATCCTGATCAACTGCAACACATCTGCCA
TCACCCAGGCTTGTCCTAAGGTGAACTTCGACCCTATCCCAATCCACTACTGCAC
ACCAGCCGGCTACGCTATCCTGAAGTGTAACAACAAGACCTTCAACGGAACCGG
CTCCTGCAACAACGTGTCTACAGTGCAGTGTACCCACGGCATCAAGCCCGTGGTG
AGCACCCAGCTGCTGCTGAACGGCAGCCTGGCTGAGGAGGAGATCATCATCCGG
TCCGAGAACCTGACAGACAACGTGAAGACCATCATCGTGCACCTGGATCAGTCC
GTGGAGATCGTGTGCACAAGGCCAAACAACAACACCGTGAAGTCTATCAGAATC
GGACCCGGCCAGACCTTCTACTACACCGGAGACATCATCGGCAACATCAGGGAG
GCCCACTGTAACATCTCTGAGAAGAAGTGGCACGAGATGCTGAGGAGAGTGAGC
GAGAAGCTGGCTGAGCACTTCCCTAACAAGACAATCAAGTTTACCAGCTCCTCTG
GCGGAGATCTGGAGATCACAACCCACAGCTTCAACTGCAGAGGAGAGTTCTTTT
ACTGTAACACCAGCGGCCTGTTTAACTCCACATACATGCCCAACGGAACCTACAT
GCCTAACGGCACAAACAACTCTAACAGCACCATCATCCTGCCCTGCAGGATCAA
GCAGATCATCAACATGTGGCAGGAAGTGGGAAGAGCCATGTACGCTCCCCCTAT
CGCCGGCAACATCACATGTAACAGCAACATCACCGGACTGCTGCTGGTGCGGGA
CGGCGGAAAGAACAACAACACAGAGATCTTCCGCCCTGGCGGAGGCGACATGAG
GGATAACTGGCGCTCCGAGCTGTACAAGTACAAGGTGGTGGAGATCAAGCCACT
GGGAGTGGCTCCAACCAGGTGCAAGAGGAGGGTGGTGGGCAGCCACTCTGGCAG
CGGAGGCTCCGGATCTGGAGGCCACGCTGCTGTGGGACTGGGAGCCGTGAGCCT
GGGATTTCTGGGAGCTGCTGGATCTACCATGGGAGCTGCTAGCATCACACTGACC
GTGCAGGCTAGGCAGCTGCTGTCCGGAATCGTGCAGCAGCAGTCTAACCTGCTGC
AGGCTCCCGAGCCTCAGCAGCACCTGCTGCAGGACACACACTGGGGCATCAAGC
AGCTGCAGACCCGCGTGCTGGCCATCGAGCACTACCTGAAGGATCAGCAGCTGC
TGGGCATCTGGGGATGTTCTGGCAAGCTGATCTGCTGTACAGCTGTGCCATGGAA
CAGCTCCTGGAGCAACAAGTCCCTGACAGACATCTGGGATAACATGACCTGGAT
GCAGTGGGATCGGGAGGTGAGCAACTACACCGGCATCATCTACCGCCTGCTGGA
AGACTCACAGAATCAGCAGGAACGGAATGAACAGGACCTCCTCGCCTGGATGG
CGGAGTCGAAAACCTGTGGGTCACCGTCTACTATGGAGTGCCAGTGTGGAAAGA
GGCTAGGACTACCCTGTTCTGTGCCAGCGATGCCAAAGCCTACGAGACAGAGGT
GCACAACGTGTGGGCAACACACGCATGCGTGCCAACCGACCCAAATCCCCAGGA
GATGGTGCTGGGCAACGTGACCGAGAACTTCAATATGTGGAAGAACGACATGGT
GGATCAGATGCACGAGGATGTGATCTCTCTGTGGGCCCAGAGCCTGAAGCCTTGC
GTGAAGCTGACCCCACTGTGCGTGACACTGGAGTGTACCCAGGTGAACGCCACA
CAGGGCAATACCACACAGGTGAACGTGACCCAAGTGAATGGCGACGAGATGAA
GAACTGTTCCTTCAATACCACAACCGAGATCAGGGATAAGAAGCAGAAGGCCTA
CGCCCTGTTTTATAGACTGGACCTGGTGCCACTGGAGAGGGAGAACAGAGGCGA
TTCTAATAGCGCCTCCAAGTATATCCTGATCAACTGCAATACATCTGCCATCACC
CAGGCCTGTCCTAAAGTGAATTTCGACCCTATCCCAATCCACTACTGCACACCAG
CCGGCTATGCCATCCTGAAGTGTAACAACAAGACCTTCAACGGCACCGGCTCCTG
CAACAACGTGAGCACAGTGCAGTGACCCACGGCATCAAGCCCGTGGTGAGCAC
CCAGCTGCTGCTGAACGGCTCCCTGGCAGAGGAGGAGATCATCATCCGGTCCGA
GAACCTGACAGACAATGTGAAGACCATCATCGTGCACCTGGATCAGTCCGTGGA
GATCGTGTGCACAAGGCCAAACAATAACACCGTGAAGTCTATCAGAATCGGCCC
CGGCCAGACCTTCTACTATACCGGCGACATCATCGGCAATATCAGGGAGGCCCA
CTGTAACATCTCTGAGAAGAAGTGGCACGAGATGCTGAGGAGAGTGAGCGAGAA
GCTGGCCGAGCACTTCCCTAATAAGACAATCAAGTTTACCAGCTCCTCTGGCGGC
GATCTGGAGATCACAACCCACAGCTTCAACTGCAGCGGCGAGTTCTTTTACTGTA
ACACCAGCGGCCTGTTTAATTCCACATACATGCCCAACGGCACCTATATGCCTAA
TGGCACAAATAACTCTAACAGCACCATCATCCTGCCCTGCAGGATCAAGCAGATC
ATCAATATGTGGCAGGAAGTGGGCAGAGCCATGTATGCCCCTCCCATCGCCGGC
AACATCACATGTAACAGCAATATCACCGGCCTGCTGCTGGTGCGGGACCGGCGGC
AAGAATAACAATACAGAGATCTTCCGCCCCGGCGGCGGCGACATGAGGGATAAC
TGGCGCTCCGAGCTGTACAAGTATAAGGTGGTGGAGATCAAGCCACTGGGAGTG
GCACCAACCAGGTGCAAGAGGCGCGTGGTGGGCTCCCACTCTGGCAGCGGCGGC
TCCGGCTCTGGCGGCCACGCAGCAGTGGGCCTGGGAGCCGTGTCCCTGGGCTTTC
TGGGAGCAGCAGGCTCTACCATGGGAGCAGCCAGCATCACACTGACCGTGCAGG
CAAGGCAGCTGCTGTCCGGCATCGTGCAGCAGCAGTCTAACCTGCTGCAGGCAC
CAGAGCCTCAGCAGCACCTGCTGCAGGACACACACTGGGGCATCAAGCAGCTGC
AGACCCGCGTGCTGGCCATCGAGCACTACCTGAAGGATCAGCAGCTGCTGGGCA
TCTGGGGCTGTTCTGGCAAGCTGATCTGCTGTACAGCCGTGCCATGGAACAGCTC
CTGGAGCAATAAGTCCCTGACAGACATCTGGGATAATATGACCTGGATGCAGTG
GGATCGGGAGGTGAGCAACTACACCGGCATCATCTATCGCCTGCTGGAGGACTC
ACAGAATCAGCAGGAGCGGAACGAACAGGATCTGCTGGCACTGGATTGATAACT
CGAG
001428_MD39_link14-nucleic acid
GGATCCGCCACCATGGACTGGACTTGGATTCTGTTCCTGGTGGCAGCAGC
AACTAGAGTGCATTCCGTCGAAAACCTGTGGGTGACCGTGTATTATGGAGTGCCC
GTGTGGAAGGAGGCCCGGACCACACTGTTCTGCGCCTCCGACGCCAAGGCCTAC
GAGACAGAGGTGCACAACGTGTGGGCCACACACGCCTGCGTGCCTACCGATCCA
AATCCCCAGGAGATGGTGCTGGGCAACGTGACCGAGAACTTTAATATGTGGAAG
AACGACATGGTGGATCAGATGCACGAGGACGTGATCTCTCTGTGGGCCCAGAGC
CTGAAGCCTTGCGTGAAGCTGACCCCACTGTGCGTGACACTGGAGTGTACCCAGG
TGAACGCCACACAGGGCAATACCACACAGGTGAACGTGACCCAAGTGAATGGCG
ACGAGATGAAGAACTGTTCCTTCAATACCACAACCGAGATCCGGGATAAGAAGC
AGAAGGCCTACGCCCTGTTTTATAGACTGGACCTGGTGCCTCTGGAGCGGGAGA
ACAGAGGCGATTCTAATAGCGCCTCCAAGTATATCCTGATCAACTGCAATACATC
TGCCATCACCCAGGCCTGTCCTAAAGTGAATTTCGATCCTATCCCAATCCACTAC
TGCACCCCAGCCGGCTATGCCATCCTGAAGTGTAACAACAAGACCTTCAACGGC
ACCGGCTCCTGCAACAACGTGAGCACAGTGCAGTGTACCCACGGCATCAAGCCA
GTGGTGAGCACCCAGCTGCTGCTGAACGGCTCCCTGGCAGAGGAGGAGATCATC
ATCAGGTCCGAGAACCTGACAGACAATGTGAAGACCATCATCGTGCACCTGGAT
CAGTCCGTGGAGATCGTGTGCACACGGCCAAACAATAACACCGTGAAGTCTATC
AGAATCGGCCCCGGCCAGACATTCTACTATACCGGCGACATCATCGGCAATATCC
GGGAGGCCCACTGTAACATCTCTGAGAAGAAGTGGCACGAGATGCTGCGGAGAG
TGAGCGAGAAGCTGGCCGAGCACTTCCCCAATAAGACAATCAAGTTTACCAGCT
CCTCTGGCGGCGATCTGGAGATCACAACCCACAGCTTCAACTGCAGAGGCGAGT
TCTTTTACTGTAACACCAGCGGCCTGTTTAATTCCACATACATGCCCAACGGCAC
CTATATGCCTAATGGCACAAATAACTCTAACAGCACCATCATCCTGCCATGCCGG
ATCAAGCAGATCATCAATATGTGGCAGGAAGTGGGCAGAGCCATGTATGCCCCT
CCCATCGCCGGCAACATCACATGTAACAGCAATATCACCGGCCTGCTGCTGGTGA
GGGACGGCGGCAAGAATAACAATACAGAGATCTTCCGCCCCGGCGGCGGCGACA
TGAGGGATAACTGGCGCTCCGAGCTGTACAAGTATAAGGTGGTGGAGATCAAGC
CACTGGGAGTGGCACCAACCAGGTGCAAGAGGCGCGTGGTGGGCTCCCACTCTG
GCAGCGGCGGCTCCGGCTCTGGCGGCCACGCAGCAGTGGGCCTGGGAGCCGTGA
GCCTGGGCTTTCTGGGAGCAGCAGGCTCTACCATGGGAGCAGCCAGCATCACAC
TGACCGTGCAGGCAAGGCAGCTGCTGTCCGGCATCGTGCAGCAGCAGTCTAACC
TGCTGCAGGCACCAGAGCCTCAGCAGCACCTGCTGCAGGACACACACTGGGGCA
TCAAGCAGCTGCAGACCCGCGTGCTGGCCATCGAGCACTACCTGAAGGATCAGC
AGCTGCTGGGCATCTGGGGCTGCTCTGGCAAGCTGATCTGCTGTACAGCCGTGCC
TTGGAACAGCTCCTGGAGCAATAAGTCCCTGACAGACATCTGGGATAATATGAC
CTGGATGCAGTGGGATAGGGAGGTGAGCAACTACACCGGCATCATCTATCGCCT
GCTGGAAGACTCACAGAATCAGCAGGAAAGGAATGAACAGGATCTGCTGGCACT
GGACTGATAACTCGAG

The disclosure relates to a composition comprising one or more nucleic acid molecules. The composition can comprise one, two, three or more nucleic acid molecules, each nucleic acid molecule comprising at least a first expressible nucleic acid sequence comprising at least one nucleic acid sequence that encodes a retroviral monomer or retorviral trimer peptide, the trimer peptide comprising at least about 70%, 75%, 80%, 85%, 90%, 95%, 96%, 97%, 98%, 99% or 100% sequence identity to one or combination of amino acid sequences selected from: SEQ ID NO: 55, SEQ ID NO: 58, SEQ ID NO: 61, SEQ ID NO: 64, SEQ ID NO: 80, SEQ ID NO: 83, SEQ ID NO: 86, SEQ ID NO: 89, SEQ ID NO: 92, SEQ ID NO: 93, SEQ ID NO: 94, SEQ ID NO: 108, SEQ ID NO: 111, SEQ ID NO: 114, SEQ ID NO: 117, SEQ ID NO: 120, SEQ ID NO: 123, SEQ ID NO: 126, SEQ ID NO: 129, SEQ ID NO: 132 or pharmaceutically acceptable salts thereof.

In some embodiments, upon administration to a subject, the composition comprising a nucleic acid comprising the expressible nucleic acid sequence is transfected or transduced into an antigen presenting cell which encodes the expressible nucleic acid sequence. After a plurality of expressible nucleic acid sequences are encoded, the first, second and third polypeptides assemble into a trimer comprising a secondary structure that exposes one or a plurality of epitopes that are not naturally exposed when the polypeptides or variants thereof are expressed under normal conditions and naturally in a host cell. Antigen presenting cells expressing the one or plurality of viral antigens can elicit a therapeutically effective antigen-specific immune response against the virus in a subject. For example, in some embodiments, the viral antigen can be an antigen from human immunodeficiency virus-1 (HIV-1).

In some embodiments, the nucleic acid sequence is an RNA sequence. In some embodiments, the RNA sequence according to the present disclosure comprises at least about 70%, 75%, 80%, 85%, 90%, 95%, 96%, 97%, 98%, 99% or more sequence identity to one or combination of RNA sequences provided in Table Y. In some embodiments, the RNA sequence according to the present disclosure comprises one or combination of RNA sequences provided in Table Y. In some embodiments, the RNA sequence according to the present disclosure comprises at least about 70%, 75%, 80%, 85%, 90%, 95%, 96%, 97%, 98%, 99% or more sequence identity to one or combination of RNA sequences selected from: SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO:256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265 or pharmaceutically acceptable salts thereof. In some embodiments, the RNA sequence according to the present disclosure comprises one or combination of RNA sequences selected from: SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, SEQ ID NO: 251, SEQ ID NO: 252, SEQ ID NO: 253, SEQ ID NO: 254, SEQ ID NO: 255, SEQ ID NO:256, SEQ ID NO: 257, SEQ ID NO: 258, SEQ ID NO: 259, SEQ ID NO: 260, SEQ ID NO: 261, SEQ ID NO: 262, SEQ ID NO: 263, SEQ ID NO: 264, SEQ ID NO: 265 or pharmaceutically acceptable salts thereof.

### 5. Regulatory Sequences

In some embodiments, the expressible nucleic acid sequence can be operably linked to one or a plurality of regulatory sequences. The term “regulatory sequence” as used herein refer to DNA sequences which are necessary to effect expression of sequences to which they are ligated. The term “regulatory sequence” is intended to include, as a minimum, all components necessary for expression and optionally additional advantageous components. In some embodiments, the regulatory sequence is a promoter sequence. As used herein, a “promoter” means a region of DNA upstream from the transcription start and which is involved in binding RNA polymerase and other proteins to start transcription. Reference herein to a “promoter” is to be taken in its broadest context and includes the transcriptional regulatory sequences derived from a classical eukaryotic genomic gene, including the TATA box which is required for accurate transcription initiation, with or without a CCAAT box sequence and additional regulatory elements (i.e. upstream activating sequences, enhancers and silencers) which alter gene expression in response to developmental and/or external stimuli, or in a tissue-specific manner. Consequently, a repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions. The term “promoter” also includes the transcriptional regulatory sequences of a classical prokaryotic gene, in which case it may include a −35 box sequence and/or a −10 box transcriptional regulatory sequences. The term “promoter” is also used to describe a synthetic or fusion molecule, or derivative which confers, activates or enhances expression of a nucleic acid molecule in a cell, tissue or organ.

### 6. Nucleic Acid Molecule

In some embodiments, the disclosed compositions further comprise a nucleic acid molecule that comprises the expressible nucleic acid sequences. For example, the nucleic acid molecule can be a plasmid. Provided herein is a vector or plasmid that is capable of expressing a at least one soluble trimer of a retroviral envelope polypeptide or constructs in the cell of a mammal in a quantity effective to elicit an immune response in the mammal. The vector may comprise heterologous nucleic acid encoding the one or more viral antigens (such as HIV-1 antigens). In some embodiments, the nucleic acid expresses a trimer of gp120, gp 41, gp160 or pharmaceutically acceptable salts or functional fragments thereof. The vector may be a plasmid. The plasmid may be useful for transfecting cells with nucleic acid encoding a viral antigen, which the transformed host cell is cultured and maintained under conditions wherein expression of the viral antigen takes place and wherein the structure of the trimer elicits an immune response of a magnitude greater than and/or more therapeutically effective than the immune response elicited by the antigen alone. The plasmid may further comprise an initiation codon, which may be upstream of the expressible sequence, and a stop codon, which may be downstream of the coding sequence. The initiation and termination codon may be in frame with the expressible sequence.

The plasmid may also comprise a promoter that is operably linked to the coding sequence. The promoter operably linked to the coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, or human metalothionein. The promoter may also be a tissue specific promoter, such as a muscle or skin specific promoter, natural or synthetic. Examples of such promoters are described in US patent application publication no. US20040175727, the contents of which are incorporated herein in its entirety. The plasmid may also comprise a polyadenylation signal, which may be downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, Calif.).

The plasmid may also comprise an enhancer upstream of the coding sequence. The enhancer may be human actin, human myosin, human hemoglobin, human muscle creatine or a viral enhancer such as one from CMV, FMDV, RSV or EBV. Polynucleotide function enhancers are described in U.S. Pat. Nos. 5,593,972, 5,962,428, and WO94/016737, the contents of each are fully incorporated by reference. The plasmid may also comprise a mammalian origin of replication in order to maintain the plasmid extrachromosomally and produce multiple copies of the plasmid in a cell. The plasmid may be pVAX1, pCEP4 or pREP4 from ThermoFisher Scientific (San Diego, Calif.), which may comprise the Epstein Barr virus origin of replication and nuclear antigen EBNA-1 coding region, which may produce high copy episomal replication without integration. The vector can be pVAX1 or a pVax1 variant with changes such as the variant plasmid described herein. The variant pVax1 plasmid is a 2998 basepair variant of the backbone vector plasmid pVAX1 (Invitrogen, Carlsbad Calif.). The CMV promoter is located at bases 137-724. The 17 promoter/priming site is at bases 664-683. Multiple cloning sites are at bases 696-811. Bovine GH polyadenylation signal is at bases 829-1053. The Kanamycin resistance gene is at bases 1226-2020. The pUC origin is at bases 2320-2993. The vaccine may comprise the consensus antigens and plasmids at quantities of from about 1 nanogram to 100 milligrams; about 1 microgram to about 10 milligrams; or preferably about 0.1 microgram to about 10 milligrams; or more preferably about 1 milligram to about 2 milligram. In some embodiments, pharmaceutical compositions according to the present invention comprise from about 1 nanogram to about 1000 micrograms of DNA, The pVAX1 plasmid sequence is as follows:

(SEQ ID NO: 229)
gactcttcgcgatgtacgggccagatatacgcgtt
gacattgattattgactagttattaatagtaatca
attacggggtcattagttcatagcccatatatgga
gttccgcgttacataacttacggtaaatggcccgc
ctggctgaccgcccaacgacccccgcccattgacg
tcaataatgacgtatgttcccatagtaacgccaat
agggactttccattgacgtcaatgggtggactatt
tacggtaaactgcccacttggcagtacatcaagtg
tatcatatgccaagtacgccccctattgacgtcaa
tgacggtaaatggcccgcctggcattatgcccagt
acatgaccttatgggactttcctacttggcagtac
atctacgtattagtcatcgctattaccatggtgat
gcggttttggcagtacatcaatgggcgtggatagc
ggtttgactcacggggatttccaagtctccacccc
attgacgtcaatgggagtttgttttggcaccaaaa
tcaacgggactttccaaaatgtcgtaacaactccg
ccccattgacgcaaatgggcggtaggcgtgtacgg
tgggaggtctatataagcagagctctctggctaac
tagagaacccactgcttactggcttatcgaaatta
atacgactcactatagggagacccaagctggctag
cgtttaaacttaagcttggtaccgagctcggatcc
actagtccagtgtggtggaattctgcagatatcca
gcacagtggcggccgctcgagtctagagggcccgt
ttaaacccgctgatcagcctcgactgtgccttcta
gttgccagccatctgttgtttgcccctcccccgtg
ccttccttgaccctggaaggtgccactcccactgt
cctttcctaataaaatgaggaaattgcatcgcatt
gtctgagtaggtgtcattctattctggggggtggg
gtggggcaggacagcaagggggaggattgggaaga
caatagcaggcatgctggggatgcggtgggctcta
tggcttctactgggcggttttatggacagcaagcg
aaccggaattgccagctggggcgccctctggtaag
gttgggaagccctgcaaagtaaactggatggcttt
ctcgccgccaaggatctgatggcgcaggggatcaa
gctctgatcaagagacaggatgaggatcgtttcgc
atgattgaacaagatggattgcacgcaggttctcc
ggccgcttgggtggagaggctattcggctatgact
gggcacaacagacaatcggctgctctgatgccgcc
gtgttccggctgtcagcgcaggggcgcccggttct
ttttgtcaagaccgacctgtccggtgccctgaatg
aactgcaagacgaggcagcgcggctatcgtggctg
gccacgacgggcgttccttgcgcagctgtgctcga
cgttgtcactgaagcgggaagggactggctgctat
tgggcgaagtgccggggcaggatctcctgtcatct
caccttgctcctgccgagaaagtatccatcatggc
tgatgcaatgcggcggctgcatacgcttgatccgg
ctacctgcccattcgaccaccaagcgaaacatcgc
atcgagcgagcacgtactcggatggaagccggtct
tgtcgatcaggatgatctggacgaagagcatcagg
ggctcgcgccagccgaactgttcgccaggctcaag
gcgagcatgcccgacggcgaggatctcgtcgtgac
ccatggcgatgcctgcttgccgaatatcatggtgg
aaaatggccgcttttctggattcatcgactgtggc
cggctgggtgtggcggaccgctatcaggacatagc
gttggctacccgtgatattgctgaagagcttggcg
gcgaatgggctgaccgcttcctcgtgctttacggt
atcgccgctcccgattcgcagcgcatcgccttcta
tcgccttcttgacgagttcttctgaattattaacg
cttacaatttcctgatgcggtattttctccttacg
catctgtgcggtatttcacaccgcatacaggtggc
acttttcggggaaatgtgcgcggaacccctatttg
tttatttttctaaatacattcaaatatgtatccgc
tcatgagacaataaccctgataaatgcttcaataa
tagcacgtgctaaaacttcatttttaatttaaaag
gatctaggtgaagatcctttttgataatctcatga
ccaaaatcccttaacgtgagttttcgttccactga
gcgtcagaccccgtagaaaagatcaaaggatcttc
ttgagatcctttttttctgcgcgtaatctgctgct
tgcaaacaaaaaaaccaccgctaccagcggtggtt
tgtttgccggatcaagagctaccaactctttttcc
gaaggtaactggcttcagcagagcgcagataccaa
atactgtccttctagtgtagccgtagttaggccac
cacttcaagaactctgtagcaccgcctacatacct
cgctctgctaatcctgttaccagtggctgctgcca
gtggcgataagtcgtgtcttaccgggttggactca
agacgatagttaccggataaggcgcagcggtcggg
ctgaacggggggttcgtgcacacagcccagcttgg
agcgaacgacctacaccgaactgagatacctacag
cgtgagctatgagaaagcgccacgcttcccgaagg
gagaaaggcggacaggtatccggtaagcggcaggg
tcggaacaggagagcgcacgagggagcttccaggg
ggaaacgcctggtatctttatagtcctgtcgggtt
tcgccacctctgacttgagcgtcgatttttgtgat
gctcgtcaggggggcggagcctatggaaaaacgcc
agcaacgcggcctttttacggttcctgggcttttg
ctggccttttgctcacatgttctt

In some embodiments, the disclosure relates to a plasmid comprising at least about 70%, 75%, 80%, 85%, 90%, 95%, 96%, 97%, 98%, 99% or 100% sequence identity to SEQ ID NO: 229, the plasmid comprising an expressible nucleic acid sequence within the multiple cloning site, and the expressible nucleic acid sequence comprising one or combination of nucleic acid sequences selected from: SEQ ID NO: 154, SEQ ID NO: 155, SEQ ID NO: 157, SEQ ID NO: 158, SEQ ID NO: 160, SEQ ID NO: 161, SEQ ID NO: 163, SEQ ID NO: 164, SEQ ID NO: 166, SEQ ID NO: 167, SEQ ID NO: 169, SEQ ID NO: 170, SEQ ID NO: 172, SEQ ID NO: 173, SEQ ID NO: 175, SEQ ID NO: 176, SEQ ID NO: 178, SEQ ID NO: 179, SEQ ID NO: 181, SEQ ID NO: 182, SEQ ID NO: 184, SEQ ID NO: 185, SEQ ID NO: 187, SEQ ID NO: 188, SEQ ID NO: 190, SEQ ID NO: 191, SEQ ID NO: 193, SEQ ID NO: 194, SEQ ID NO: 196, SEQ ID NO: 197, SEQ ID NO: 199, SEQ ID NO: 200, SEQ ID NO: 202, SEQ ID NO: 203, SEQ ID NO: 205, SEQ ID NO: 206, SEQ ID NO: 208, SEQ ID NO: 209, SEQ ID NO: 211, SEQ ID NO: 212, SEQ ID NO: 214, SEQ ID NO: 215, SEQ ID NO: 217, SEQ ID NO: 218, SEQ ID NO: 220, SEQ ID NO: 221, SEQ ID NO: 223, SEQ ID NO: 224, SEQ ID NO: 226, SEQ ID NO: 227, pharmaceutically acceptable salts thereof; or nucleic acid sequences that comprise at least about 70%, 75%, 80%, 85%, 90%, 95%, 96%, 97%, 98%, 99% or 100% sequence identity to any one or combination of nucleic acid sequences disclosed from SEQ ID NO: 53 through SEQ ID NO: 131.

In some embodiments, the plasmid comprises an expressible nucleic acid sequence comprising at least about 70%, 75%, 80%, 85%, 90%, 95%, 96%, 97%, 98%, 99% or 100% sequence identity to SEQ ID NO: 239 or a pharmaceutically acceptable salt thereof.

(SEQ ID NO: 239)
ATGGACTGGACCTGGATTCTGTTCCTGGTGGCCGC
CGCCACAAGGGTGCACAGCATGCAGATCTACGAAG
GAAAACTGACCGCTGAGGGACTGAGGTTCGGAATT
GTCGCAAGCCGCGCGAATCACGCACTGGTGGATAG
GCTGGTGGAAGGCGCTATCGACGCAATTGTCCGGC
ACGGCGGGAGAGAGGAAGACATCACACTGGTGAGA
GTCTGCGGCAGCTGGGAGATTCCCGTGGCAGCTGG
AGAACTGGCTCGAAAGGAGGACATCGATGCCGTGA
TCGCTATTGGGGTCCTGTGCCGAGGAGCAACTCCC
AGCTTCGACTACATCGCCTCAGAAGTGAGCAAGGG
GCTGGCTGATCTGTCCCTGGAGCTGAGGAAACCTA
TCACTTTTGGCGTGATTACTGCCGACACCCTGGAA
CAGGCAATCGAGGCGGCCGGCACCTGCCATGGAAA
CAAAGGCTGGGAAGCAGCCCTGTGCGCTATTGAGA
TGGCAAATCTGTTCAAATCTCTGCGAGGAGGCTCC
GGAGGATCTGGAGGGAGTGGAGGCTCAGGAGGAGG
CGACACCATCACACTGCCATGCCGCCCTGCACCAC
CTCCACATTGTAGCTCCAACATCACCGGCCTGATT
CTGACAAGACAGGGGGGATATAGTAACGATAATAC
CGTGATTTTCAGGCCCTCAGGAGGGGACTGGAGGG
ACATCGCACGATGCCAGATTGCTGGAACAGTGGTC
TCTACTCAGCTGTTTCTGAACGGCAGTCTGGCTGA
GGAAGAGGTGGTCATCCGATCTGAAGACTGGCGGG
ATAATGCAAAGTCAATTTGTGTGCAGCTGAACACA
AGCGTCGAGATCAATTGCACTGGCGCAGGGCACTG
TAACATTTCTCGGGCCAAATGGAACAATACCCTGA
AGCAGATCGCCAGTAAACTGAGAGAGCAGTACGGC
AATAAGACAATCATCTTCAAGCCTTCTAGTGGAGG
CGACCCAGAGTTCGTGAACCATAGCTTTAATTGCG
GGGGAGAGTTCTTTTATTGTGATTCCACACAGCTG
TTCAACAGCACTTGGTTTAATTCCACCTGATAA

Thus, in some embodiments, the disclosed compositions can be vectors comprising a DNA backbone with an expressible insert comprising one or more of the disclosed leader sequences, self-assembling polypeptides, linkers and/or viral antigens.

The disclosure relates to a nucleic acid sequence comprising at least one expressible nucleic acid sequence comprising in a 5′ to 3′ orientation, a leader sequence, retroviral trimer sequence and, optionally, a transmembrane domain. The disclosure relates to a nucleic acid sequence comprising at least one expressible nucleic acid sequence comprising in a 5′ to 3′ orientation, a leader sequence, retroviral trimer sequence and, optionally, a foldon domain. In some embodiments, the at least one expressible nucleic acid sequence comprising in a 5′ to 3′ orientation, a leader sequence, retroviral trimer sequence and, optionally, a transmembrane domain and a foldon domain. In some embodiments, the transmembrane membrane domain encodes a platelet derived growth factor receptor or functional fragment thereof that comprises at least about 70%, 75%, 80%, 85%, 90%, 95%, 96%, 97%, 98% 99% or 100% sequence identity or sequence homology to

(SEQ ID NO: 240)
AVGQDTQEVIVVPHSLPFKVVVISAILALVVLTI
ISLIILIMLWQKKPR.

In some embodiments, the expressible nucleic acid encodes a foldon domain or functional fragment thereof that comprises at least about 70%, 75%, 80%, 85%, 90%, 95%, 96%, 97%, 98% 99% or 100% sequence identity or sequence homology to

(SEQ ID NO: 235)
YIPEAPRDGQAYVRKDGEWVLLSTFL.

The disclosure also relates to a composition (such as a pharmaceutical composition) comprising a nucleic acid molecule comprising at least one nucleic acid expressible nucleic acid sequence that encodes one or more retorviral monomers. In some embodiments, the nucleic acid molecule comprises at least a first nucleic acid sequence comprising a first, second, a third domain, each domain encoding a retroviral monomer, and each monomer independently selected from: an amino acid or functional fragment thereof that comprises at least about 70%, 75%, 80%, 85%, 90%, 95%, 96%, 97%, 98% 99% or 100% sequence identity or sequence homology to those amino acids from, through and between SEQ ID NO: 55 through SEQ ID NO: 132.

In some embodiments, the composition comprises an expressible nucleic acid sequence comprising three retroviral monomer sequences, the sequence encoding an amino acid sequence or functional fragment that is at least about 70%, 75%, 80%, 85%, 90%, 95%, 96%, 97%, 98% 99% or 100% sequence identity or sequence homology to those amino acids from, through and between SEQ ID NO: 156 through SEQ ID NO: 228. In some embodiments, the composition comprises an expressible nucleic acid sequence comprising three retroviral monomer sequences, the sequence encoding an amino acid sequence or functional fragment that is at least about 70%, 75%, 80%, 85%, 90%, 95%, 96%, 97%, 98% 99% or 100% sequence identity or sequence homology to a sequence identified as a sequence MD39. In some embodiments, the composition comprises an expressible nucleic acid sequence comprising three retroviral monomer sequences, the sequence encoding an amino acid sequence or functional fragment that is at least about 70%, 75%, 80%, 85%, 90%, 95%, 96%, 97%, 98% 99% or 100% sequence identity or sequence homology to BG505. In some embodiments, the composition comprises an expressible nucleic acid sequence comprising three retroviral monomer sequences, the sequence encoding an amino acid sequence or functional fragment that is at least about 70%, 75%, 80%, 85%, 90%, 95%, 96%, 97%, 98% 99% or 100% sequence identity or sequence homology to a sequence identified as a sequence TRO11. In some embodiments, the composition comprises an expressible nucleic acid sequence comprising three retroviral monomer sequences, the sequence encoding an amino acid sequence or functional fragment that is at least about 70%, 75%, 80%, 85%, 90%, 95%, 96%, 97%, 98% 99% or 100% sequence identity or sequence homology to a sequence identified as a sequence AY835445. In some embodiments, the composition comprises an expressible nucleic acid sequence comprising three retroviral monomer sequences, the sequence encoding an amino acid sequence or functional fragment that is at least about 70%, 75%, 80%, 85%, 90%, 95%, 96%, 97%, 98% 99% or 100% sequence identity or sequence homology to a sequence identified as a sequence X2278.

In some embodiments, the composition comprises an expressible nucleic acid sequence comprising three retroviral monomer sequences, a first monomer sequence encoding an amino acid sequence or functional fragment that is at least about 70%, 75%, 80%, 85%, 90%, 95%, 96%, 97%, 98% 99% or 100% sequence identity or sequence homology to SEQ ID NO: 55 through SEQ ID NO: 228, a second monomer encoding an amino acid sequence or functional fragment that is at least about 70%, 75%, 80%, 85%, 90%, 95%, 96%, 97%, 98% 99% or 100% sequence identity or sequence homology to SEQ ID NO: 156 through SEQ ID NO: 228, and a third monomer sequence encoding an amino acid sequence or functional fragment that is at least about 70%, 75%, 80%, 85%, 90%, 95%, 96%, 97%, 98% 99% or 100% sequence identity or sequence homology to SEQ ID NO: 55 through SEQ ID NO: 228. In some embodiments, each of the retorviral monomer sequences are linked by one or more linker sequences.

In some embodiments, the composition is a pharmaceutical composition comprising SEQ ID NO identified as a leader and a nucleic acid sequence comprising at least about 70%, 75%, 80%, 85%, 90%, 95%, 96%, 97%, 98% 99% or 100% sequence identity or sequence homology to a nucleic acid sequence from, through and between SEQ ID NO: 53 to SEQ ID NO: 228, wherein, within the multiple cloning site the nucleic acid molecule further comprise at least on expressible nucleic acid sequence operably linked to a promoter sequence, the expressible nucleic acid sequence comprising:

(i) one or a combination of nucleic acid sequences chosen from a leader sequence disclosed herein; or

(ii) one or a combination of nucleic acid sequences wherein the at least one nucleic acid sequence comprises at least about 70%, 75%, 80%, 85%, 90%, 95%, 96%, 97%, 98%, 99% or 100% sequence identity to a sequence chosen from: SEQ ID NO: 154, SEQ ID NO: 155, SEQ ID NO: 157, SEQ ID NO: 158, SEQ ID NO: 160, SEQ ID NO: 161, SEQ ID NO: 163, SEQ ID NO: 164, SEQ ID NO: 166, SEQ ID NO: 167, SEQ ID NO: 169, SEQ ID NO: 170, SEQ ID NO: 172, SEQ ID NO: 173, SEQ ID NO: 175, SEQ ID NO: 176, SEQ ID NO: 178, SEQ ID NO: 179, SEQ ID NO: 181, SEQ ID NO: 182, SEQ ID NO: 184, SEQ ID NO: 185, SEQ ID NO: 187, SEQ ID NO: 188, SEQ ID NO: 190, SEQ ID NO: 191, SEQ ID NO: 193, SEQ ID NO: 194, SEQ ID NO: 196, SEQ ID NO: 197, SEQ ID NO: 199, SEQ ID NO: 200, SEQ ID NO: 202, SEQ ID NO: 203, SEQ ID NO: 205, SEQ ID NO: 206, SEQ ID NO: 208, SEQ ID NO: 209, SEQ ID NO: 211, SEQ ID NO: 212, SEQ ID NO: 214, SEQ ID NO: 215, SEQ ID NO: 217, SEQ ID NO: 218, SEQ ID NO: 220, SEQ ID NO: 221, SEQ ID NO: 223, SEQ ID NO: 224, SEQ ID NO: 226, SEQ ID NO: 227; or

(iii) one or a combination of nucleic acid sequences that encode an amino acid sequence chosen from: SEQ ID NO: 55, SEQ ID NO: 58, SEQ ID NO: 61, SEQ ID NO: 64, SEQ ID NO: 80, SEQ ID NO: 83, SEQ ID NO: 86, SEQ ID NO: 89, SEQ ID NO: 92, SEQ ID NO: 93, SEQ ID NO: 94, SEQ ID NO: 108, SEQ ID NO: 111, SEQ ID NO: 114, SEQ ID NO: 117, SEQ ID NO: 120, SEQ ID NO: 123, SEQ ID NO: 126, SEQ ID NO: 129, SEQ ID NO: 132; or

(iv) one or a combination of nucleic acid sequences that encode at least one amino acid sequences comprising at least about 70%, 75%, 80%, 85%, 90%, 95%, 96%, 97%, 98%, 99% or 100% sequence identity to a sequence chosen from: a linker sequence disclosed herein.

In some embodiments, the expressible nucleic acid sequence comprises RNA. Exemplary RNA sequences of the disclosure are one or a combination of nucleic acid sequences that comprise at least about 70%, 80%, 85%, 90%, 95%, 96%, 97%, 98%, 99%, 100% sequence identity to a sequence chosen from:

BG505_SOSIP_MD39_trimer string 1-RNA
(SEQ ID NO: 241)
AUGGACUGGACAUGGAUUCUGUUCCUGGUCGCUGC
CGCUACAAGAGUGCAUUCCGCCGAAAACCUGUGGG
UCACCGUCUACUAUGGAGUGCCCGUGUGGAAGGAC
GCCGAGACUACGCUGUUCUGCGCCAGCGAUGCCAA
GGCCUACGAGACAGAGAAGCACAACGUGUGGGCAA
CCCACGCAUGCGUGCCUACAGACCCAAACCCCCAG
GAGAUCCACCUGGAGAAUGUGACAGAGGAGUUUAA
CAUGUGGAAGAACAAUAUGGUGGAGCAGAUGCACG
AGGACAUCAUCUCCCUGUGGGAUCAGUCUCUGAAG
CCCUGCGUGAAGCUGACCCCUCUGUGCGUGACACU
GCAGUGUACCAACGUGACAAACAAUAUCACCGACG
AUAUGCGGGGCGAGCUGAAGAAUUGUAGCUUCAAC
AUGACCACAGAGCUGAGGGACAAGAAGCAGAAGGU
GUACUCCCUGUUUUAUAGACUGGAUGUGGUGCAGA
UCAAUGAGAACCAGGGCAAUCGGUCUAACAAUAGC
AACAAGGAGUACCGCCUGAUCAAUUGCAACACCUC
CGCCAUCACACAGGCCUGUCCUAAGGUGUCUUUCG
AGCCUAUCCCAAUCCACUAUUGCGCCCCAGCCGGC
UUCGCCAUCCUGAAGUGUAAGGAUAAGAAGUUUAA
CGGAACCGGACCAUGCCCUUCCGUGUCUACCGUGC
AGUGUACACACGGCAUCAAGCCUGUGGUGUCUACA
CAGCUGCUGCUGAAUGGCAGCCUGGCCGAGGAGGA
AGUGAUCAUCAGGUCUGAGAACAUCACCAACAAUG
CCAAGAAUAUCCUGGUGCAGCUGAACACACCAGUG
CAGAUCAAUUGCACCCGGCCCAACAAUAACACAGU
GAAGUCUAUCCGCAUCGGCCCAGGCCAGGCCUUUU
ACUAUACCGGCGACAUCAUCGGCGAUAUCAGACAG
GCCCACUGUAAUGUGAGCAAGGCCACCUGGAACGA
GACACUGGGCAAGGUGGUGAAGCAGCUGAGGAAGC
ACUUCGGCAAUAACACCAUCAUCAGAUUUGCACAG
AGCUCCGGCGGCGACCUGGAGGUGACCACACACUC
CUUCAAUUGCGGCGGCGAGUUCUUUUACUGUAACA
CAAGCGGCCUGUUUAAUUCCACCUGGAUCUCCAAC
ACAUCUGUGCAGGGCAGCAAUUCCACCGGCAGCAA
CGAUUCCAUCACACUGCCAUGCCGGAUCAAGCAGA
UCAUCAACAUGUGGCAGCGCAUCGGCCAGGCCAUG
UAUGCCCCUCCCAUCCAGGGCGUGAUCAGAUGCGU
GAGCAAUAUCACCGGCCUGAUCCUGACACGCGACG
GCGGCUCUACCAACAGCACCACAGAGACAUUCCGG
CCCGGCGGCGGCGACAUGAGGGAUAACUGGAGAUC
UGAGCUGUACAAGUAUAAGGUGGUGAAGAUCGAGC
CUCUGGGAGUGGCACCAACCAGGUGCAAGAGGAGA
GUGGUGGGCUCUCACAGCGGCUCCGGCGGCUCUGG
CAGCGGCGGCCACGCCGCAGUGGGCAUCGGAGCCG
UGUCCCUGGGCUUUCUGGGAGCAGCAGGCUCCACA
AUGGGAGCAGCCUCUAUGACCCUGACAGUGCAGGC
CAGGAAUCUGCUGAGCGGCAUCGUGCAGCAGCAGU
CCAACCUGCUGAGAGCCCCAGAGCCCCAGCAGCAC
CUGCUGAAGGACACCCACUGGGGCAUCAAGCAGCU
GCAGGCCAGGGUGCUGGCAGUGGAGCACUAUCUGA
GAGAUCAGCAGCUGCUGGGCAUCUGGGGCUGUAGC
GGCAAGCUGAUCUGCUGUACCAAUGUGCCCUGGAA
CUCUAGCUGGUCUAAUCGCAACCUGAGCGAGAUCU
GGGACAAUAUGACCUGGCUGCAGUGGGAUAAGGAG
AUCUCCAACUACACACAGAUCAUCUAUGGCCUGCU
GGAAGAAUCUCAGAAUCAGCAGGAAAAGAAUGAAC
AGGAUCUGCUGGCACUGGAUGGCGGCGCCGAAAAC
CUGUGGGUCACCGUGUACUACGGAGUCCCCGUGUG
GAAAGAUGCAGAGACAACCCUGUUCUGCGCUUCCG
ACGCUAAAGCUUACGAGACAGAAAAACACAACGUG
UGGGCCACUCAUGCCUGCGUGCCUACAGACCCUAA
CCCACAGGAAAUCCACCUGGAGAAUGUGACGGAGG
AGUUUAACAUGUGGAAGAAUAACAUGGUCGAGCAG
AUGCAUGAAGAUAUCAUUUCCUUAUGGGACCAAUC
CCUGAAGCCUUGCGUGAAGCUGACCCCACUGUGCG
UGACACUGCAAUGCACUAACGUGACCAAUAACAUU
ACCGACGAUAUGCGCGGCGAGCUGAAGAACUGCUC
UUUCAACAUGACUACCGAGCUGAGAGAUAAGAAAC
AGAAAGUGUACAGCCUGUUUUAUCGGUUAGAUGUG
GUGCAGAUCAAUGAAAACCAGGGCAAUCGGUCCAA
CAAUUCUAACAAGGAAUAUCGCCUGAUCAAUUGUA
ACACCUCCGCCAUUACCCAGGCUUGCCCUAAGGUG
UCUUUCGAGCCCAUCCCUAUCCACUAUUGCGCCCC
AGCUGGAUUUGCUAUCCUGAAGUGUAAGGACAAAA
AGUUUAACGGGACCGGACCAUGUCCUAGCGUGUCC
ACUGUGCAGUGCACCCAUGGCAUCAAGCCUGUGGU
GUCCACCCAACUUCUGCUGAAUGGCUCUCUGGCUG
AAGAAGAAGUGAUCAUUAGGUCCGAAAAUAUUACU
AAUAACGCUAAAAAUAUCCUGGUCCAGCUGAACAC
GCCUGUCCAGAUCAAUUGUACCCGGCCAAAUAACA
ACACAGUGAAGUCUAUCAGAAUCGGCCCAGGCCAG
GCCUUCUACUACACAGGCGACAUUAUCGGCGAUAU
UCGCCAGGCCCACUGUAAUGUGAGCAAAGCUACAU
GGAAUGAGACACUGGGCAAGGUAGUCAAACAGCUG
AGAAAACAUUUUGGAAACAACACCAUCAUCCGCUU
UGCACAGUCUAGCGGCGGCGACCUGGAGGUAACUA
CCCACAGCUUCAAUUGUGGCGGCGAGUUCUUUUAC
UGUAAUACCAGCGGCCUGUUUAAUAGUACUUGGAU
CAGCAACACAUCUGUGCAGGGCUCUAACUCCACUG
GCUCUAACGAUAGCAUCACACUGCCUUGUCGGAUC
AAGCAAAUCAUCAACAUGUGGCAAAGGAUUGGGCA
GGCUAUGUAUGCCCCUCCAAUCCAGGGCGUGAUCC
GGUGCGUGAGCAACAUUACAGGCCUGAUCCUGACA
AGAGACGGCGGCUCCACCAACUCUACUACCGAGAC
AUUCCGGCCCGGCGGCGGCGACAUGCGUGAUAACU
GGCGCAGCGAACUGUAUAAAUAUAAAGUGGUGAAG
AUCGAGCCUCUGGGCGUGGCCCCAACUAGGUGUAA
AAGAAGGGUCGUCGGCUCCCACAGCGGCAGCGGCG
GCUCCGGCUCUGGCGGCCACGCGGCUGUCGGCAUC
GGCGCCGUGAGCCUGGGCUUUCUGGGCGCCGCCGG
CUCCACUAUGGGCGCAGCCUCUAUGACCCUGACUG
UCCAGGCUAGAAAUCUGCUGUCUGGAAUCGUGCAG
CAGCAGUCUAACCUGCUGAGGGCACCUGAGCCACA
ACAGCACCUGCUGAAGGAUACACAUUGGGGCAUCA
AGCAGUUACAAGCCAGGGUGCUGGCCGUGGAACAC
UACCUGCGCGAUCAGCAAUUACUGGGCAUUUGGGG
AUGCUCUGGCAAGCUGAUUUGUUGCACCAAUGUGC
CCUGGAACUCCUCUUGGAGCAACAGAAACCUGUCC
GAAAUCUGGGAUAACAUGACAUGGCUGCAGUGGGA
CAAGGAAAUUUCCAAUUAUACCCAGAUCAUCUAUG
GACUGCUGGAAGAAAGUCAGAAUCAGCAGGAGAAG
AAUGAACAGGAUCUGCUGGCACUGGAUGGCGGCGC
CGAAAACCUGUGGGUCACCGUGUAUUAUGGAGUGC
CAGUGUGGAAGGACGCCGAGACCACACUGUUUUGU
GCCUCUGAUGCCAAGGCCUACGAGACCGAGAAGCA
CAACGUGUGGGCCACCCACGCCUGCGUGCCCACAG
ACCCAAAUCCUCAGGAGAUCCACCUGGAGAACGUG
ACCGAGGAGUUUAACAUGUGGAAGAACAAUAUGGU
GGAGCAGAUGCACGAGGAUAUCAUCUCUCUGUGGG
AUCAGUCUCUGAAGCCAUGUGUGAAGCUGACCCCA
CUGUGCGUGACCCUGCAGUGUACAAAUGUGACAAA
CAACAUCACAGAUGACAUGAGAGGCGAGCUGAAGA
ACUGUUCCUUCAAUAUGACCACCGAGCUGAGAGAC
AAGAAGCAGAAGGUGUAUUCUCUGUUUUACCGGCU
GGACGUGGUGCAGAUCAACGAGAAUCAGGGCAAUC
GGUCUAACAACUCCAAUAAGGAGUAUAGACUGAUC
AACUGCAACACCUCUGCCAUCACCCAGGCCUGUCC
UAAGGUGUCCUUUGAGCCAAUCCCAAUCCACUAUU
GCGCCCCUGCCGGCUUUGCCAUCCUGAAGUGCAAG
GACAAGAAGUUUAACGGCACAGGCCCCUGCCCAUC
CGUGAGCACAGUGCAGUGUACCCACGGCAUCAAGC
CUGUGGUGUCCACCCAGCUGCUGCUGAACGGCUCC
CUGGCCGAGGAGGAGGUAAUCAUCAGGUCUGAGAA
CAUCACAAAUAACGCCAAGAACAUCCUGGUGCAGC
UGAACACCCCAGUGCAGAUCAACUGUACCCGGCCU
AACAAUAAUACCGUGAAGUCUAUCCGGAUCGGCCC
AGGCCAGGCCUUCUACUAUACCGGCGAUAUCAUCG
GCGAUAUCAGACAGGCCCACUGCAACGUGUCCAAG
GCCACAUGGAACGAGACACUGGGCAAGGUGGUGAA
GCAGCUGCGGAAGCACUUUGGCAAUAACACCAUCA
UCAGAUUCGCCCAGUCUUCCGGCGGCGACCUGGAG
GUGACAACCCACUCCUUCAAUUGCGGCGGCGAGUU
CUUUUACUGUAAUACAAGCGGCCUGUUUAAUAGCA
CCUGGAUCUCUAACACCUCCGUGCAGGGCUCCAAC
AGCACAGGCUCUAAUGAUUCCAUCACCCUGCCUUG
CCGGAUCAAGCAGAUCAUCAAUAUGUGGCAGAGAA
UCGGCCAGGCCAUGUAUGCCCCUCCAAUCCAGGGC
GUGAUCCGCUGCGUGUCCAACAUCACAGGCCUGAU
CCUGACAAGAGAUGGCGGCUCCACCAACAGCACCA
CAGAGACCUUCAGACCCGGCGGCGGCGACAUGCGC
GACAACUGGAGAUCCGAGCUGUAUAAGUACAAGGU
GGUGAAGAUCGAGCCCCUGGGCGUGGCCCCAACCC
GGUGUAAGCGCAGAGUGGUGGGCAGCCACAGCGGC
AGCGGCGGCAGCGGCUCCGGCGGCCACGCCGCCGU
GGGCAUCGGCGCCGUGUCCCUGGGCUUCCUGGGCG
CCGCCGGCUCCACCAUGGGCGCCGCCUCCAUGACA
CUGACAGUGCAGGCCAGAAAUCUGCUGUCCGGCAU
CGUGCAGCAGCAGUCCAAUCUGCUGCGGGCCCCUG
AGCCACAGCAGCACCUGCUGAAGGAUACCCACUGG
GGCAUCAAGCAGCUGCAGGCCCGGGUGCUGGCCGU
GGAGCACUACCUGAGGGAUCAGCAGCUGCUGGGCA
UCUGGGGCUGUUCCGGCAAGCUGAUCUGCUGUACA
AACGUGCCCUGGAACAGCUCCUGGUCCAAUAGGAA
CCUGUCCGAGAUCUGGGAUAACAUGACCUGGCUGC
AGUGGGAUAAGGAGAUCAGCAACUACACACAGAUC
AUCUACGGCCUGCUGGAGGAGAGCCAGAAUCAGCA
GGAGAAGAACGAGCAGGACCUGCUGGCCCUGGAU
BG505_SOSIP_MD39_trimer string 2-RNA
(SEQ ID NO: 242)
GGAUCCGCCACCAUGGACUGGACAUGGAUUCUGUU
CCUGGUCGCUGCCGCUACAAGAGUGCAUUCCGCCG
AAAACCUGUGGGUCACCGUCUACUAUGGAGUGCCC
GUGUGGAAGGACGCCGAGACUACGCUGUUCUGCGC
CAGCGAUGCCAAGGCCUACGAGACAGAGAAGCACA
ACGUGUGGGCAACCCACGCAUGCGUGCCUACAGAC
CCAAACCCCCAGGAGAUCCACCUGGAGAAUGUGAC
AGAGGAGUUUAACAUGUGGAAGAACAAUAUGGUGG
AGCAGAUGCACGAGGACAUCAUCUCCCUGUGGGAU
CAGUCUCUGAAGCCCUGCGUGAAGCUGACCCCUCU
GUGCGUGACACUGCAGUGUACCAACGUGACAAACA
AUAUCACCGACGAUAUGCGGGGCGAGCUGAAGAAU
UGUAGCUUCAACAUGACCACAGAGCUGAGGGACAA
GAAGCAGAAGGUGUACUCCCUGUUUUAUAGACUGG
AUGUGGUGCAGAUCAAUGAGAACCAGGGCAAUCGG
UCUAACAAUAGCAACAAGGAGUACCGCCUGAUCAA
UUGCAACACCUCCGCCAUCACACAGGCCUGUCCUA
AGGUGUCUUUCGAGCCUAUCCCAAUCCACUAUUGC
GCCCCAGCCGGCUUCGCCAUCCUGAAGUGUAAGGA
UAAGAAGUUUAACGGAACCGGACCAUGCCCUUCCG
UGUCUACCGUGCAGUGUACACACGGCAUCAAGCCU
GUGGUGUCUACACAGCUGCUGCUGAAUGGCAGCCU
GGCCGAGGAGGAAGUGAUCAUCAGGUCUGAGAACA
UCACCAACAAUGCCAAGAAUAUCCUGGUGCAGCUG
AACACACCAGUGCAGAUCAAUUGCACCCGGCCCAA
CAAUAACACAGUGAAGUCUAUCCGCAUCGGCCCAG
GCCAGGCCUUUUACUAUACCGGCGACAUCAUCGGC
GAUAUCAGACAGGCCCACUGUAAUGUGAGCAAGGC
CACCUGGAACGAGACACUGGGCAAGGUGGUGAAGC
AGCUGAGGAAGCACUUCGGCAAUAACACCAUCAUC
AGAUUUGCACAGAGCUCCGGCGGCGACCUGGAGGU
GACCACACACUCCUUCAAUUGCGGCGGCGAGUUCU
UUUACUGUAACACAAGCGGCCUGUUUAAUUCCACC
UGGAUCUCCAACACAUCUGUGCAGGGCAGCAAUUC
CACCGGCAGCAACGAUUCCAUCACACUGCCAUGCC
GGAUCAAGCAGAUCAUCAACAUGUGGCAGCGCAUC
GGCCAGGCCAUGUAUGCCCCUCCCAUCCAGGGCGU
GAUCAGAUGCGUGAGCAAUAUCACCGGCCUGAUCC
UGACACGCGACGGCGGCUCUACCAACAGCACCACA
GAGACAUUCCGGCCCGGCGGCGGCGACAUGAGGGA
UAACUGGAGAUCUGAGCUGUACAAGUAUAAGGUGG
UGAAGAUCGAGCCUCUGGGAGUGGCACCAACCAGG
UGCAAGAGGAGAGUGGUGGGCUCUCACAGCGGCUC
CGGCGGCUCUGGCAGCGGCGGCCACGCCGCAGUGG
GCAUCGGAGCCGUGUCCCUGGGCUUUCUGGGAGCA
GCAGGCUCCACAAUGGGAGCAGCCUCUAUGACCCU
GACAGUGCAGGCCAGGAAUCUGCUGAGCGGCAUCG
UGCAGCAGCAGUCCAACCUGCUGAGAGCCCCAGAG
CCCCAGCAGCACCUGCUGAAGGACACCCACUGGGG
CAUCAAGCAGCUGCAGGCCAGGGUGCUGGCAGUGG
AGCACUAUCUGAGAGAUCAGCAGCUGCUGGGCAUC
UGGGGCUGUAGCGGCAAGCUGAUCUGCUGUACCAA
UGUGCCCUGGAACUCUAGCUGGUCUAAUCGCAACC
UGAGCGAGAUCUGGGACAAUAUGACCUGGCUGCAG
UGGGAUAAGGAGAUCUCCAACUACACACAGAUCAU
CUAUGGCCUGCUGGAAGAAUCUCAGAAUCAGCAGG
AAAAGAAUGAACAGGAUCUGCUGGCACUGGAUGGC
GGCAGCGGCAGCGGCGCCGAAAACCUGUGGGUCAC
CGUGUACUACGGAGUCCCCGUGUGGAAAGAUGCAG
AGACAACCCUGUUCUGCGCUUCCGACGCUAAAGCU
UACGAGACAGAAAAACACAACGUGUGGGCCACUCA
UGCCUGCGUGCCUACAGACCCUAACCCACAGGAAA
UCCACCUGGAGAAUGUGACGGAGGAGUUUAACAUG
UGGAAGAAUAACAUGGUCGAGCAGAUGCAUGAAGA
UAUCAUUUCCUUAUGGGACCAAUCCCUGAAGCCUU
GCGUGAAGCUGACCCCACUGUGCGUGACACUGCAA
UGCACUAACGUGACCAAUAACAUUACCGACGAUAU
GCGCGGCGAGCUGAAGAACUGCUCUUUCAACAUGA
CUACCGAGCUGAGAGAUAAGAAACAGAAAGUGUAC
AGCCUGUUUUAUCGGUUAGAUGUGGUGCAGAUCAA
UGAAAACCAGGGCAAUCGGUCCAACAAUUCUAACA
AGGAAUAUCGCCUGAUCAAUUGUAACACCUCCGCC
AUUACCCAGGCUUGCCCUAAGGUGUCUUUCGAGCC
CAUCCCUAUCCACUAUUGCGCCCCAGCUGGAUUUG
CUAUCCUGAAGUGUAAGGACAAAAAGUUUAACGGG
ACCGGACCAUGUCCUAGCGUGUCCACUGUGCAGUG
CACCCAUGGCAUCAAGCCUGUGGUGUCCACCCAAC
UUCUGCUGAAUGGCUCUCUGGCUGAAGAAGAAGUG
AUCAUUAGGUCCGAAAAUAUUACUAAUAACGCUAA
AAAUAUCCUGGUCCAGCUGAACACGCCUGUCCAGA
UCAAUUGUACCCGGCCAAAUAACAACACAGUGAAG
UCUAUCAGAAUCGGCCCAGGCCAGGCCUUCUACUA
CACAGGCGACAUUAUCGGCGAUAUUCGCCAGGCCC
ACUGUAAUGUGAGCAAAGCUACAUGGAAUGAGACA
CUGGGCAAGGUAGUCAAACAGCUGAGAAAACAUUU
UGGAAACAACACCAUCAUCCGCUUUGCACAGUCUA
GCGGCGGCGACCUGGAGGUAACUACCCACAGCUUC
AAUUGUGGCGGCGAGUUCUUUUACUGUAAUACCAG
CGGCCUGUUUAAUAGUACUUGGAUCAGCAACACAU
CUGUGCAGGGCUCUAACUCCACUGGCUCUAACGAU
AGCAUCACACUGCCUUGUCGGAUCAAGCAAAUCAU
CAACAUGUGGCAAAGGAUUGGGCAGGCUAUGUAUG
CCCCUCCAAUCCAGGGCGUGAUCCGGUGCGUGAGC
AACAUUACAGGCCUGAUCCUGACAAGAGACGGCGG
CUCCACCAACUCUACUACCGAGACAUUCCGGCCCG
GCGGCGGCGACAUGCGUGAUAACUGGCGCAGCGAA
CUGUAUAAAUAUAAAGUGGUGAAGAUCGAGCCUCU
GGGCGUGGCCCCAACUAGGUGUAAAAGAAGGGUCG
UCGGCUCCCACAGCGGCAGCGGCGGCUCCGGCUCU
GGCGGCCACGCGGCUGUCGGCAUCGGCGCCGUGAG
CCUGGGCUUUCUGGGCGCCGCCGGCUCCACUAUGG
GCGCAGCCUCUAUGACCCUGACUGUCCAGGCUAGA
AAUCUGCUGUCUGGAAUCGUGCAGCAGCAGUCUAA
CCUGCUGAGGGCACCUGAGCCACAACAGCACCUGC
UGAAGGAUACACAUUGGGGCAUCAAGCAGUUACAA
GCCAGGGUGCUGGCCGUGGAACACUACCUGCGCGA
UCAGCAAUUACUGGGCAUUUGGGGAUGCUCUGGCA
AGCUGAUUUGUUGCACCAAUGUGCCCUGGAACUCC
UCUUGGAGCAACAGAAACCUGUCCGAAAUCUGGGA
UAACAUGACAUGGCUGCAGUGGGACAAGGAAAUUU
CCAAUUAUACCCAGAUCAUCUAUGGACUGCUGGAA
GAAAGUCAGAAUCAGCAGGAGAAGAAUGAACAGGA
UCUGCUGGCACUGGAUGGCGGCAGCGGCAGCGGCG
CCGAAAACCUGUGGGUCACCGUGUAUUAUGGAGUG
CCAGUGUGGAAGGACGCCGAGACCACACUGUUUUG
UGCCUCUGAUGCCAAGGCCUACGAGACCGAGAAGC
ACAACGUGUGGGCCACCCACGCCUGCGUGCCCACA
GACCCAAAUCCUCAGGAGAUCCACCUGGAGAACGU
GACCGAGGAGUUUAACAUGUGGAAGAACAAUAUGG
UGGAGCAGAUGCACGAGGAUAUCAUCUCUCUGUGG
GAUCAGUCUCUGAAGCCAUGUGUGAAGCUGACCCC
ACUGUGCGUGACCCUGCAGUGUACAAAUGUGACAA
ACAACAUCACAGAUGACAUGAGAGGCGAGCUGAAG
AACUGUUCCUUCAAUAUGACCACCGAGCUGAGAGA
CAAGAAGCAGAAGGUGUAUUCUCUGUUUUACCGGC
UGGACGUGGUGCAGAUCAACGAGAAUCAGGGCAAU
CGGUCUAACAACUCCAAUAAGGAGUAUAGACUGAU
CAACUGCAACACCUCUGCCAUCACCCAGGCCUGUC
CUAAGGUGUCCUUUGAGCCAAUCCCAAUCCACUAU
UGCGCCCCUGCCGGCUUUGCCAUCCUGAAGUGCAA
GGACAAGAAGUUUAACGGCACAGGCCCCUGCCCAU
CCGUGAGCACAGUGCAGUGUACCCACGGCAUCAAG
CCUGUGGUGUCCACCCAGCUGCUGCUGAACGGCUC
CCUGGCCGAGGAGGAGGUAAUCAUCAGGUCUGAGA
ACAUCACAAAUAACGCCAAGAACAUCCUGGUGCAG
CUGAACACCCCAGUGCAGAUCAACUGUACCCGGCC
UAACAAUAAUACCGUGAAGUCUAUCCGGAUCGGCC
CAGGCCAGGCCUUCUACUAUACCGGCGAUAUCAUC
GGCGAUAUCAGACAGGCCCACUGCAACGUGUCCAA
GGCCACAUGGAACGAGACACUGGGCAAGGUGGUGA
AGCAGCUGCGGAAGCACUUUGGCAAUAACACCAUC
AUCAGAUUCGCCCAGUCUUCCGGCGGCGACCUGGA
GGUGACAACCCACUCCUUCAAUUGCGGCGGCGAGU
UCUUUUACUGUAAUACAAGCGGCCUGUUUAAUAGC
ACCUGGAUCUCUAACACCUCCGUGCAGGGCUCCAA
CAGCACAGGCUCUAAUGAUUCCAUCACCCUGCCUU
GCCGGAUCAAGCAGAUCAUCAAUAUGUGGCAGAGA
AUCGGCCAGGCCAUGUAUGCCCCUCCAAUCCAGGG
CGUGAUCCGCUGCGUGUCCAACAUCACAGGCCUGA
UCCUGACAAGAGAUGGCGGCUCCACCAACAGCACC
ACAGAGACCUUCAGACCCGGCGGCGGCGACAUGCG
CGACAACUGGAGAUCCGAGCUGUAUAAGUACAAGG
UGGUGAAGAUCGAGCCCCUGGGCGUGGCCCCAACC
CGGUGUAAGCGCAGAGUGGUGGGCAGCCACAGCGG
CAGCGGCGGCAGCGGCUCCGGCGGCCACGCCGCCG
UGGGCAUCGGCGCCGUGUCCCUGGGCUUCCUGGGC
GCCGCCGGCUCCACCAUGGGCGCCGCCUCCAUGAC
ACUGACAGUGCAGGCCAGAAAUCUGCUGUCCGGCA
UCGUGCAGCAGCAGUCCAAUCUGCUGCGGGCCCCU
GAGCCACAGCAGCACCUGCUGAAGGAUACCCACUG
GGGCAUCAAGCAGCUGCAGGCCCGGGUGCUGGCCG
UGGAGCACUACCUGAGGGAUCAGCAGCUGCUGGGC
AUCUGGGGCUGUUCCGGCAAGCUGAUCUGCUGUAC
AAACGUGCCCUGGAACAGCUCCUGGUCCAAUAGGA
ACCUGUCCGAGAUCUGGGAUAACAUGACCUGGCUG
CAGUGGGAUAAGGAGAUCAGCAACUACACACAGAU
CAUCUACGGCCUGCUGGAGGAGAGCCAGAAUCAGC
AGGAGAAGAACGAGCAGGACCUGCUGGCCCUGGAU
UGAUAACUCGAG
(22) BG505_MD39_link14_gp140-PDGFR
(SEQ ID NO: 243)
AUGGACUGGACAUGGAUUCUGUUCCUGGUCGCUGC
CGCUACAAGAGUGCAUUCCGCCGAAAACCUGUGGG
UCACCGUCUACUAUGGAGUGCCCGUGUGGAAGGAC
GCCGAGACUACGCUGUUCUGCGCCAGCGAUGCCAA
GGCCUACGAGACAGAGAAGCACAACGUGUGGGCAA
CCCACGCAUGCGUGCCUACAGACCCAAACCCCCAG
GAGAUCCACCUGGAGAAUGUGACAGAGGAGUUUAA
CAUGUGGAAGAACAAUAUGGUGGAGCAGAUGCACG
AGGACAUCAUCUCCCUGUGGGAUCAGUCUCUGAAG
CCCUGCGUGAAGCUGACCCCUCUGUGCGUGACACU
GCAGUGUACCAACGUGACAAACAAUAUCACCGACG
AUAUGCGGGGCGAGCUGAAGAAUUGUAGCUUCAAC
AUGACCACAGAGCUGAGGGACAAGAAGCAGAAGGU
GUACUCCCUGUUUUAUAGACUGGAUGUGGUGCAGA
UCAAUGAGAACCAGGGCAAUCGGUCUAACAAUAGC
AACAAGGAGUACCGCCUGAUCAAUUGCAACACCUC
CGCCAUCACACAGGCCUGUCCUAAGGUGUCUUUCG
AGCCUAUCCCAAUCCACUAUUGCGCCCCAGCCGGC
UUCGCCAUCCUGAAGUGUAAGGAUAAGAAGUUUAA
CGGAACCGGACCAUGCCCUUCCGUGUCUACCGUGC
AGUGUACACACGGCAUCAAGCCUGUGGUGUCUACA
CAGCUGCUGCUGAAUGGCAGCCUGGCCGAGGAGGA
AGUGAUCAUCAGGUCUGAGAACAUCACCAACAAUG
CCAAGAAUAUCCUGGUGCAGCUGAACACACCAGUG
CAGAUCAAUUGCACCCGGCCCAACAAUAACACAGU
GAAGUCUAUCCGCAUCGGCCCAGGCCAGGCCUUUU
ACUAUACCGGCGACAUCAUCGGCGAUAUCAGACAG
GCCCACUGUAAUGUGAGCAAGGCCACCUGGAACGA
GACACUGGGCAAGGUGGUGAAGCAGCUGAGGAAGC
ACUUCGGCAAUAACACCAUCAUCAGAUUUGCACAG
AGCUCCGGCGGCGACCUGGAGGUGACCACACACUC
CUUCAAUUGCGGCGGCGAGUUCUUUUACUGUAACA
CAAGCGGCCUGUUUAAUUCCACCUGGAUCUCCAAC
ACAUCUGUGCAGGGCAGCAAUUCCACCGGCAGCAA
CGAUUCCAUCACACUGCCAUGCCGGAUCAAGCAGA
UCAUCAACAUGUGGCAGCGCAUCGGCCAGGCCAUG
UAUGCCCCUCCCAUCCAGGGCGUGAUCAGAUGCGU
GAGCAAUAUCACCGGCCUGAUCCUGACACGCGACG
GCGGCUCUACCAACAGCACCACAGAGACAUUCCGG
CCCGGCGGCGGCGACAUGAGGGAUAACUGGAGAUC
UGAGCUGUACAAGUAUAAGGUGGUGAAGAUCGAGC
CUCUGGGAGUGGCACCAACCAGGUGCAAGAGGAGA
GUGGUGGGCUCUCACAGCGGCUCCGGCGGCUCUGG
CAGCGGCGGCCACGCCGCAGUGGGCAUCGGAGCCG
UGUCCCUGGGCUUUCUGGGAGCAGCAGGCUCCACA
AUGGGAGCAGCCUCUAUGACCCUGACAGUGCAGGC
CAGGAAUCUGCUGAGCGGCAUCGUGCAGCAGCAGU
CCAACCUGCUGAGAGCCCCAGAGCCCCAGCAGCAC
CUGCUGAAGGACACCCACUGGGGCAUCAAGCAGCU
GCAGGCCAGGGUGCUGGCAGUGGAGCACUAUCUGA
GAGAUCAGCAGCUGCUGGGCAUCUGGGGCUGUAGC
GGCAAGCUGAUCUGCUGUACCAAUGUGCCCUGGAA
CUCUAGCUGGUCUAAUCGCAACCUGAGCGAGAUCU
GGGACAAUAUGACCUGGCUGCAGUGGGAUAAGGAG
AUCUCCAACUACACACAGAUCAUCUAUGGCCUGCU
GGAAGAAUCUCAGAAUCAGCAGGAAAAGAAUGAAC
AGGAUCUGCUGGCACUGGAUGGAGGAGGAAGCGGG
GGAAGCGGGGGAAGCGGAGGAAGCGGGGGAAGCGG
GGGAAGCAACGCCGUGGGCCAGGACACCCAGGAAG
UGAUCGUGGUGCCCCACAGCCUGCCUUUCAAGGUG
GUGGUCAUCUCCGCCAUCCUGGCCCUGGUCGUGCU
GACUAUUAUUUCCCUGAUUAUCCUGAUUAUGCUGU
GGCAGAAGAAGCCCAGA
BG505_MD39_gp140_foldon-PDGFR
(SEQ ID NO: 244)
AUGGACUGGACAUGGAUUCUGUUCCUGGUCGCUGC
CGCUACAAGAGUGCAUUCCGCCGAAAACCUGUGGG
UCACCGUCUACUAUGGAGUGCCCGUGUGGAAGGAC
GCCGAGACUACGCUGUUCUGCGCCAGCGAUGCCAA
GGCCUACGAGACAGAGAAGCACAACGUGUGGGCAA
CCCACGCAUGCGUGCCUACAGACCCAAACCCCCAG
GAGAUCCACCUGGAGAAUGUGACAGAGGAGUUUAA
CAUGUGGAAGAACAAUAUGGUGGAGCAGAUGCACG
AGGACAUCAUCUCCCUGUGGGAUCAGUCUCUGAAG
CCCUGCGUGAAGCUGACCCCUCUGUGCGUGACACU
GCAGUGUACCAACGUGACAAACAAUAUCACCGACG
AUAUGCGGGGCGAGCUGAAGAAUUGUAGCUUCAAC
AUGACCACAGAGCUGAGGGACAAGAAGCAGAAGGU
GUACUCCCUGUUUUAUAGACUGGAUGUGGUGCAGA
UCAAUGAGAACCAGGGCAAUCGGUCUAACAAUAGC
AACAAGGAGUACCGCCUGAUCAAUUGCAACACCUC
CGCCAUCACACAGGCCUGUCCUAAGGUGUCUUUCG
AGCCUAUCCCAAUCCACUAUUGCGCCCCAGCCGGC
UUCGCCAUCCUGAAGUGUAAGGAUAAGAAGUUUAA
CGGAACCGGACCAUGCCCUUCCGUGUCUACCGUGC
AGUGUACACACGGCAUCAAGCCUGUGGUGUCUACA
CAGCUGCUGCUGAAUGGCAGCCUGGCCGAGGAGGA
AGUGAUCAUCAGGUCUGAGAACAUCACCAACAAUG
CCAAGAAUAUCCUGGUGCAGCUGAACACACCAGUG
CAGAUCAAUUGCACCCGGCCCAACAAUAACACAGU
GAAGUCUAUCCGCAUCGGCCCAGGCCAGGCCUUUU
ACUAUACCGGCGACAUCAUCGGCGAUAUCAGACAG
GCCCACUGUAAUGUGAGCAAGGCCACCUGGAACGA
GACACUGGGCAAGGUGGUGAAGCAGCUGAGGAAGC
ACUUCGGCAAUAACACCAUCAUCAGAUUUGCACAG
AGCUCCGGCGGCGACCUGGAGGUGACCACACACUC
CUUCAAUUGCGGCGGCGAGUUCUUUUACUGUAACA
CAAGCGGCCUGUUUAAUUCCACCUGGAUCUCCAAC
ACAUCUGUGCAGGGCAGCAAUUCCACCGGCAGCAA
CGAUUCCAUCACACUGCCAUGCCGGAUCAAGCAGA
UCAUCAACAUGUGGCAGCGCAUCGGCCAGGCCAUG
UAUGCCCCUCCCAUCCAGGGCGUGAUCAGAUGCGU
GAGCAAUAUCACCGGCCUGAUCCUGACACGCGACG
GCGGCUCUACCAACAGCACCACAGAGACAUUCCGG
CCCGGCGGCGGCGACAUGAGGGAUAACUGGAGAUC
UGAGCUGUACAAGUAUAAGGUGGUGAAGAUCGAGC
CUCUGGGAGUGGCACCAACCAGGUGCAAGAGGAGA
GUGGUGGGCUCUCACAGCGGCUCCGGCGGCUCUGG
CAGCGGCGGCCACGCCGCAGUGGGCAUCGGAGCCG
UGUCCCUGGGCUUUCUGGGAGCAGCAGGCUCCACA
AUGGGAGCAGCCUCUAUGACCCUGACAGUGCAGGC
CAGGAAUCUGCUGAGCGGCAUCGUGCAGCAGCAGU
CCAACCUGCUGAGAGCCCCAGAGCCCCAGCAGCAC
CUGCUGAAGGACACCCACUGGGGCAUCAAGCAGCU
GCAGGCCAGGGUGCUGGCAGUGGAGCACUAUCUGA
GAGAUCAGCAGCUGCUGGGCAUCUGGGGCUGUAGC
GGCAAGCUGAUCUGCUGUACCAAUGUGCCCUGGAA
CUCUAGCUGGUCUAAUCGCAACCUGAGCGAGAUCU
GGGACAAUAUGACCUGGCUGCAGUGGGAUAAGGAG
AUCUCCAACUACACACAGAUCAUCUAUGGCCUGCU
GGAAGAAUCUCAGAAUCAGCAGGAAAAGAAUGAAC
AGGAUCUGCUGGCACUGGAUGGAGGAGGAAGCGGG
GGAAGCGGCGGCGGCUACAUCCCUGAGGCCCCAAG
GGACGGACAGGCCUAUGUGAGAAAGGAUGGCGAGU
GGGUGCUGCUGUCCACCUUCCUGGGGGGAAGCGGA
GGAAGCGGGGGAAGCGGGGGAAGCAACGCCGUGGG
CCAGGACACCCAGGAAGUGAUCGUGGUGCCCCACA
GCCUGCCUUUCAAGGUGGUGGUCAUCUCCGCCAUC
CUGGCCCUGGUCGUGCUGACUAUUAUUUCCCUGAU
UAUCCUGAUUAUGCUGUGGCAGAAGAAGCCCAGA
BG505_MD39_TS1_gp140-PDGFR
(SEQ ID NO: 245)
GGAUCCGCCACCAUGGACUGGACAUGGAUUCUGUU
CCUGGUCGCUGCCGCUACAAGAGUGCAUUCCGCCG
AAAACCUGUGGGUCACCGUCUACUAUGGAGUGCCC
GUGUGGAAGGACGCCGAGACUACGCUGUUCUGCGC
CAGCGAUGCCAAGGCCUACGAGACAGAGAAGCACA
ACGUGUGGGCAACCCACGCAUGCGUGCCUACAGAC
CCAAACCCCCAGGAGAUCCACCUGGAGAAUGUGAC
AGAGGAGUUUAACAUGUGGAAGAACAAUAUGGUGG
AGCAGAUGCACGAGGACAUCAUCUCCCUGUGGGAU
CAGUCUCUGAAGCCCUGCGUGAAGCUGACCCCUCU
GUGCGUGACACUGCAGUGUACCAACGUGACAAACA
AUAUCACCGACGAUAUGCGGGGCGAGCUGAAGAAU
UGUAGCUUCAACAUGACCACAGAGCUGAGGGACAA
GAAGCAGAAGGUGUACUCCCUGUUUUAUAGACUGG
AUGUGGUGCAGAUCAAUGAGAACCAGGGCAAUCGG
UCUAACAAUAGCAACAAGGAGUACCGCCUGAUCAA
UUGCAACACCUCCGCCAUCACACAGGCCUGUCCUA
AGGUGUCUUUCGAGCCUAUCCCAAUCCACUAUUGC
GCCCCAGCCGGCUUCGCCAUCCUGAAGUGUAAGGA
UAAGAAGUUUAACGGAACCGGACCAUGCCCUUCCG
UGUCUACCGUGCAGUGUACACACGGCAUCAAGCCU
GUGGUGUCUACACAGCUGCUGCUGAAUGGCAGCCU
GGCCGAGGAGGAAGUGAUCAUCAGGUCUGAGAACA
UCACCAACAAUGCCAAGAAUAUCCUGGUGCAGCUG
AACACACCAGUGCAGAUCAAUUGCACCCGGCCCAA
CAAUAACACAGUGAAGUCUAUCCGCAUCGGCCCAG
GCCAGGCCUUUUACUAUACCGGCGACAUCAUCGGC
GAUAUCAGACAGGCCCACUGUAAUGUGAGCAAGGC
CACCUGGAACGAGACACUGGGCAAGGUGGUGAAGC
AGCUGAGGAAGCACUUCGGCAAUAACACCAUCAUC
AGAUUUGCACAGAGCUCCGGCGGCGACCUGGAGGU
GACCACACACUCCUUCAAUUGCGGCGGCGAGUUCU
UUUACUGUAACACAAGCGGCCUGUUUAAUUCCACC
UGGAUCUCCAACACAUCUGUGCAGGGCAGCAAUUC
CACCGGCAGCAACGAUUCCAUCACACUGCCAUGCC
GGAUCAAGCAGAUCAUCAACAUGUGGCAGCGCAUC
GGCCAGGCCAUGUAUGCCCCUCCCAUCCAGGGCGU
GAUCAGAUGCGUGAGCAAUAUCACCGGCCUGAUCC
UGACACGCGACGGCGGCUCUACCAACAGCACCACA
GAGACAUUCCGGCCCGGCGGCGGCGACAUGAGGGA
UAACUGGAGAUCUGAGCUGUACAAGUAUAAGGUGG
UGAAGAUCGAGCCUCUGGGAGUGGCACCAACCAGG
UGCAAGAGGAGAGUGGUGGGCUCUCACAGCGGCUC
CGGCGGCUCUGGCAGCGGCGGCCACGCCGCAGUGG
GCAUCGGAGCCGUGUCCCUGGGCUUUCUGGGAGCA
GCAGGCUCCACAAUGGGAGCAGCCUCUAUGACCCU
GACAGUGCAGGCCAGGAAUCUGCUGAGCGGCAUCG
UGCAGCAGCAGUCCAACCUGCUGAGAGCCCCAGAG
CCCCAGCAGCACCUGCUGAAGGACACCCACUGGGG
CAUCAAGCAGCUGCAGGCCAGGGUGCUGGCAGUGG
AGCACUAUCUGAGAGAUCAGCAGCUGCUGGGCAUC
UGGGGCUGUAGCGGCAAGCUGAUCUGCUGUACCAA
UGUGCCCUGGAACUCUAGCUGGUCUAAUCGCAACC
UGAGCGAGAUCUGGGACAAUAUGACCUGGCUGCAG
UGGGAUAAGGAGAUCUCCAACUACACACAGAUCAU
CUAUGGCCUGCUGGAAGAAUCUCAGAAUCAGCAGG
AAAAGAAUGAACAGGAUCUGCUGGCACUGGAUGGC
GGCGCCGAAAACCUGUGGGUCACCGUGUACUACGG
AGUCCCCGUGUGGAAAGAUGCAGAGACAACCCUGU
UCUGCGCUUCCGACGCUAAAGCUUACGAGACAGAA
AAACACAACGUGUGGGCCACUCAUGCCUGCGUGCC
UACAGACCCUAACCCACAGGAAAUCCACCUGGAGA
AUGUGACGGAGGAGUUUAACAUGUGGAAGAAUAAC
AUGGUCGAGCAGAUGCAUGAAGAUAUCAUUUCCUU
AUGGGACCAAUCCCUGAAGCCUUGCGUGAAGCUGA
CCCCACUGUGCGUGACACUGCAAUGCACUAACGUG
ACCAAUAACAUUACCGACGAUAUGCGCGGCGAGCU
GAAGAACUGCUCUUUCAACAUGACUACCGAGCUGA
GAGAUAAGAAACAGAAAGUGUACAGCCUGUUUUAU
CGGUUAGAUGUGGUGCAGAUCAAUGAAAACCAGGG
CAAUCGGUCCAACAAUUCUAACAAGGAAUAUCGCC
UGAUCAAUUGUAACACCUCCGCCAUUACCCAGGCU
UGCCCUAAGGUGUCUUUCGAGCCCAUCCCUAUCCA
CUAUUGCGCCCCAGCUGGAUUUGCUAUCCUGAAGU
GUAAGGACAAAAAGUUUAACGGGACCGGACCAUGU
CCUAGCGUGUCCACUGUGCAGUGCACCCAUGGCAU
CAAGCCUGUGGUGUCCACCCAACUUCUGCUGAAUG
GCUCUCUGGCUGAAGAAGAAGUGAUCAUUAGGUCC
GAAAAUAUUACUAAUAACGCUAAAAAUAUCCUGGU
CCAGCUGAACACGCCUGUCCAGAUCAAUUGUACCC
GGCCAAAUAACAACACAGUGAAGUCUAUCAGAAUC
GGCCCAGGCCAGGCCUUCUACUACACAGGCGACAU
UAUCGGCGAUAUUCGCCAGGCCCACUGUAAUGUGA
GCAAAGCUACAUGGAAUGAGACACUGGGCAAGGUA
GUCAAACAGCUGAGAAAACAUUUUGGAAACAACAC
CAUCAUCCGCUUUGCACAGUCUAGCGGCGGCGACC
UGGAGGUAACUACCCACAGCUUCAAUUGUGGCGGC
GAGUUCUUUUACUGUAAUACCAGCGGCCUGUUUAA
UAGUACUUGGAUCAGCAACACAUCUGUGCAGGGCU
CUAACUCCACUGGCUCUAACGAUAGCAUCACACUG
CCUUGUCGGAUCAAGCAAAUCAUCAACAUGUGGCA
AAGGAUUGGGCAGGCUAUGUAUGCCCCUCCAAUCC
AGGGCGUGAUCCGGUGCGUGAGCAACAUUACAGGC
CUGAUCCUGACAAGAGACGGCGGCUCCACCAACUC
UACUACCGAGACAUUCCGGCCCGGCGGCGGCGACA
UGCGUGAUAACUGGCGCAGCGAACUGUAUAAAUAU
AAAGUGGUGAAGAUCGAGCCUCUGGGCGUGGCCCC
AACUAGGUGUAAAAGAAGGGUCGUCGGCUCCCACA
GCGGCAGCGGCGGCUCCGGCUCUGGCGGCCACGCG
GCUGUCGGCAUCGGCGCCGUGAGCCUGGGCUUUCU
GGGCGCCGCCGGCUCCACUAUGGGCGCAGCCUCUA
UGACCCUGACUGUCCAGGCUAGAAAUCUGCUGUCU
GGAAUCGUGCAGCAGCAGUCUAACCUGCUGAGGGC
ACCUGAGCCACAACAGCACCUGCUGAAGGAUACAC
AUUGGGGCAUCAAGCAGUUACAAGCCAGGGUGCUG
GCCGUGGAACACUACCUGCGCGAUCAGCAAUUACU
GGGCAUUUGGGGAUGCUCUGGCAAGCUGAUUUGUU
GCACCAAUGUGCCCUGGAACUCCUCUUGGAGCAAC
AGAAACCUGUCCGAAAUCUGGGAUAACAUGACAUG
GCUGCAGUGGGACAAGGAAAUUUCCAAUUAUACCC
AGAUCAUCUAUGGACUGCUGGAAGAAAGUCAGAAU
CAGCAGGAGAAGAAUGAACAGGAUCUGCUGGCACU
GGAUGGCGGCGCCGAAAACCUGUGGGUCACCGUGU
AUUAUGGAGUGCCAGUGUGGAAGGACGCCGAGACC
ACACUGUUUUGUGCCUCUGAUGCCAAGGCCUACGA
GACCGAGAAGCACAACGUGUGGGCCACCCACGCCU
GCGUGCCCACAGACCCAAAUCCUCAGGAGAUCCAC
CUGGAGAACGUGACCGAGGAGUUUAACAUGUGGAA
GAACAAUAUGGUGGAGCAGAUGCACGAGGAUAUCA
UCUCUCUGUGGGAUCAGUCUCUGAAGCCAUGUGUG
AAGCUGACCCCACUGUGCGUGACCCUGCAGUGUAC
AAAUGUGACAAACAACAUCACAGAUGACAUGAGAG
GCGAGCUGAAGAACUGUUCCUUCAAUAUGACCACC
GAGCUGAGAGACAAGAAGCAGAAGGUGUAUUCUCU
GUUUUACCGGCUGGACGUGGUGCAGAUCAACGAGA
AUCAGGGCAAUCGGUCUAACAACUCCAAUAAGGAG
UAUAGACUGAUCAACUGCAACACCUCUGCCAUCAC
CCAGGCCUGUCCUAAGGUGUCCUUUGAGCCAAUCC
CAAUCCACUAUUGCGCCCCUGCCGGCUUUGCCAUC
CUGAAGUGCAAGGACAAGAAGUUUAACGGCACAGG
CCCCUGCCCAUCCGUGAGCACAGUGCAGUGUACCC
ACGGCAUCAAGCCUGUGGUGUCCACCCAGCUGCUG
CUGAACGGCUCCCUGGCCGAGGAGGAGGUAAUCAU
CAGGUCUGAGAACAUCACAAAUAACGCCAAGAACA
UCCUGGUGCAGCUGAACACCCCAGUGCAGAUCAAC
UGUACCCGGCCUAACAAUAAUACCGUGAAGUCUAU
CCGGAUCGGCCCAGGCCAGGCCUUCUACUAUACCG
GCGAUAUCAUCGGCGAUAUCAGACAGGCCCACUGC
AACGUGUCCAAGGCCACAUGGAACGAGACACUGGG
CAAGGUGGUGAAGCAGCUGCGGAAGCACUUUGGCA
AUAACACCAUCAUCAGAUUCGCCCAGUCUUCCGGC
GGCGACCUGGAGGUGACAACCCACUCCUUCAAUUG
CGGCGGCGAGUUCUUUUACUGUAAUACAAGCGGCC
UGUUUAAUAGCACCUGGAUCUCUAACACCUCCGUG
CAGGGCUCCAACAGCACAGGCUCUAAUGAUUCCAU
CACCCUGCCUUGCCGGAUCAAGCAGAUCAUCAAUA
UGUGGCAGAGAAUCGGCCAGGCCAUGUAUGCCCCU
CCAAUCCAGGGCGUGAUCCGCUGCGUGUCCAACAU
CACAGGCCUGAUCCUGACAAGAGAUGGCGGCUCCA
CCAACAGCACCACAGAGACCUUCAGACCCGGCGGC
GGCGACAUGCGCGACAACUGGAGAUCCGAGCUGUA
UAAGUACAAGGUGGUGAAGAUCGAGCCCCUGGGCG
UGGCCCCAACCCGGUGUAAGCGCAGAGUGGUGGGC
AGCCACAGCGGCAGCGGCGGCAGCGGCUCCGGCGG
CCACGCCGCCGUGGGCAUCGGCGCCGUGUCCCUGG
GCUUCCUGGGCGCCGCCGGCUCCACCAUGGGCGCC
GCCUCCAUGACACUGACAGUGCAGGCCAGAAAUCU
GCUGUCCGGCAUCGUGCAGCAGCAGUCCAAUCUGC
UGCGGGCCCCUGAGCCACAGCAGCACCUGCUGAAG
GAUACCCACUGGGGCAUCAAGCAGCUGCAGGCCCG
GGUGCUGGCCGUGGAGCACUACCUGAGGGAUCAGC
AGCUGCUGGGCAUCUGGGGCUGUUCCGGCAAGCUG
AUCUGCUGUACAAACGUGCCCUGGAACAGCUCCUG
GUCCAAUAGGAACCUGUCCGAGAUCUGGGAUAACA
UGACCUGGCUGCAGUGGGAUAAGGAGAUCAGCAAC
UACACACAGAUCAUCUACGGCCUGCUGGAGGAGAG
CCAGAAUCAGCAGGAGAAGAACGAGCAGGACCUGC
UGGCCCUGGAUGGAGGAGGAAGCGGGGGAAGCGGG
GGAAGCGGAGGAAGCGGGGGAAGCGGGGGAAGCAA
CGCCGUGGGCCAGGACACCCAGGAAGUGAUCGUGG
UGCCCCACAGCCUGCCUUUCAAGGUGGUGGUCAUC
UCCGCCAUCCUGGCCCUGGUCGUGCUGACUAUUAU
UUCCCUGAUUAUCCUGAUUAUGCUGUGGCAGAAGA
AGCCCAGA
BG505_MD39_TS1_gp140-PDGFR
(SEQ ID NO: 246)
GGAUCCGCCACCAUGGACUGGACAUGGAUUCUGUU
CCUGGUCGCUGCCGCUACAAGAGUGCAUUCCGCCG
AAAACCUGUGGGUCACCGUCUACUAUGGAGUGCCC
GUGUGGAAGGACGCCGAGACUACGCUGUUCUGCGC
CAGCGAUGCCAAGGCCUACGAGACAGAGAAGCACA
ACGUGUGGGCAACCCACGCAUGCGUGCCUACAGAC
CCAAACCCCCAGGAGAUCCACCUGGAGAAUGUGAC
AGAGGAGUUUAACAUGUGGAAGAACAAUAUGGUGG
AGCAGAUGCACGAGGACAUCAUCUCCCUGUGGGAU
CAGUCUCUGAAGCCCUGCGUGAAGCUGACCCCUCU
GUGCGUGACACUGCAGUGUACCAACGUGACAAACA
AUAUCACCGACGAUAUGCGGGGCGAGCUGAAGAAU
UGUAGCUUCAACAUGACCACAGAGCUGAGGGACAA
GAAGCAGAAGGUGUACUCCCUGUUUUAUAGACUGG
AUGUGGUGCAGAUCAAUGAGAACCAGGGCAAUCGG
UCUAACAAUAGCAACAAGGAGUACCGCCUGAUCAA
UUGCAACACCUCCGCCAUCACACAGGCCUGUCCUA
AGGUGUCUUUCGAGCCUAUCCCAAUCCACUAUUGC
GCCCCAGCCGGCUUCGCCAUCCUGAAGUGUAAGGA
UAAGAAGUUUAACGGAACCGGACCAUGCCCUUCCG
UGUCUACCGUGCAGUGUACACACGGCAUCAAGCCU
GUGGUGUCUACACAGCUGCUGCUGAAUGGCAGCCU
GGCCGAGGAGGAAGUGAUCAUCAGGUCUGAGAACA
UCACCAACAAUGCCAAGAAUAUCCUGGUGCAGCUG
AACACACCAGUGCAGAUCAAUUGCACCCGGCCCAA
CAAUAACACAGUGAAGUCUAUCCGCAUCGGCCCAG
GCCAGGCCUUUUACUAUACCGGCGACAUCAUCGGC
GAUAUCAGACAGGCCCACUGUAAUGUGAGCAAGGC
CACCUGGAACGAGACACUGGGCAAGGUGGUGAAGC
AGCUGAGGAAGCACUUCGGCAAUAACACCAUCAUC
AGAUUUGCACAGAGCUCCGGCGGCGACCUGGAGGU
GACCACACACUCCUUCAAUUGCGGCGGCGAGUUCU
UUUACUGUAACACAAGCGGCCUGUUUAAUUCCACC
UGGAUCUCCAACACAUCUGUGCAGGGCAGCAAUUC
CACCGGCAGCAACGAUUCCAUCACACUGCCAUGCC
GGAUCAAGCAGAUCAUCAACAUGUGGCAGCGCAUC
GGCCAGGCCAUGUAUGCCCCUCCCAUCCAGGGCGU
GAUCAGAUGCGUGAGCAAUAUCACCGGCCUGAUCC
UGACACGCGACGGCGGCUCUACCAACAGCACCACA
GAGACAUUCCGGCCCGGCGGCGGCGACAUGAGGGA
UAACUGGAGAUCUGAGCUGUACAAGUAUAAGGUGG
UGAAGAUCGAGCCUCUGGGAGUGGCACCAACCAGG
UGCAAGAGGAGAGUGGUGGGCUCUCACAGCGGCUC
CGGCGGCUCUGGCAGCGGCGGCCACGCCGCAGUGG
GCAUCGGAGCCGUGUCCCUGGGCUUUCUGGGAGCA
GCAGGCUCCACAAUGGGAGCAGCCUCUAUGACCCU
GACAGUGCAGGCCAGGAAUCUGCUGAGCGGCAUCG
UGCAGCAGCAGUCCAACCUGCUGAGAGCCCCAGAG
CCCCAGCAGCACCUGCUGAAGGACACCCACUGGGG
CAUCAAGCAGCUGCAGGCCAGGGUGCUGGCAGUGG
AGCACUAUCUGAGAGAUCAGCAGCUGCUGGGCAUC
UGGGGCUGUAGCGGCAAGCUGAUCUGCUGUACCAA
UGUGCCCUGGAACUCUAGCUGGUCUAAUCGCAACC
UGAGCGAGAUCUGGGACAAUAUGACCUGGCUGCAG
UGGGAUAAGGAGAUCUCCAACUACACACAGAUCAU
CUAUGGCCUGCUGGAAGAAUCUCAGAAUCAGCAGG
AAAAGAAUGAACAGGAUCUGCUGGCACUGGAUGGC
GGCGCCGAAAACCUGUGGGUCACCGUGUACUACGG
AGUCCCCGUGUGGAAAGAUGCAGAGACAACCCUGU
UCUGCGCUUCCGACGCUAAAGCUUACGAGACAGAA
AAACACAACGUGUGGGCCACUCAUGCCUGCGUGCC
UACAGACCCUAACCCACAGGAAAUCCACCUGGAGA
AUGUGACGGAGGAGUUUAACAUGUGGAAGAAUAAC
AUGGUCGAGCAGAUGCAUGAAGAUAUCAUUUCCUU
AUGGGACCAAUCCCUGAAGCCUUGCGUGAAGCUGA
CCCCACUGUGCGUGACACUGCAAUGCACUAACGUG
ACCAAUAACAUUACCGACGAUAUGCGCGGCGAGCU
GAAGAACUGCUCUUUCAACAUGACUACCGAGCUGA
GAGAUAAGAAACAGAAAGUGUACAGCCUGUUUUAU
CGGUUAGAUGUGGUGCAGAUCAAUGAAAACCAGGG
CAAUCGGUCCAACAAUUCUAACAAGGAAUAUCGCC
UGAUCAAUUGUAACACCUCCGCCAUUACCCAGGCU
UGCCCUAAGGUGUCUUUCGAGCCCAUCCCUAUCCA
CUAUUGCGCCCCAGCUGGAUUUGCUAUCCUGAAGU
GUAAGGACAAAAAGUUUAACGGGACCGGACCAUGU
CCUAGCGUGUCCACUGUGCAGUGCACCCAUGGCAU
CAAGCCUGUGGUGUCCACCCAACUUCUGCUGAAUG
GCUCUCUGGCUGAAGAAGAAGUGAUCAUUAGGUCC
GAAAAUAUUACUAAUAACGCUAAAAAUAUCCUGGU
CCAGCUGAACACGCCUGUCCAGAUCAAUUGUACCC
GGCCAAAUAACAACACAGUGAAGUCUAUCAGAAUC
GGCCCAGGCCAGGCCUUCUACUACACAGGCGACAU
UAUCGGCGAUAUUCGCCAGGCCCACUGUAAUGUGA
GCAAAGCUACAUGGAAUGAGACACUGGGCAAGGUA
GUCAAACAGCUGAGAAAACAUUUUGGAAACAACAC
CAUCAUCCGCUUUGCACAGUCUAGCGGCGGCGACC
UGGAGGUAACUACCCACAGCUUCAAUUGUGGCGGC
GAGUUCUUUUACUGUAAUACCAGCGGCCUGUUUAA
UAGUACUUGGAUCAGCAACACAUCUGUGCAGGGCU
CUAACUCCACUGGCUCUAACGAUAGCAUCACACUG
CCUUGUCGGAUCAAGCAAAUCAUCAACAUGUGGCA
AAGGAUUGGGCAGGCUAUGUAUGCCCCUCCAAUCC
AGGGCGUGAUCCGGUGCGUGAGCAACAUUACAGGC
CUGAUCCUGACAAGAGACGGCGGCUCCACCAACUC
UACUACCGAGACAUUCCGGCCCGGCGGCGGCGACA
UGCGUGAUAACUGGCGCAGCGAACUGUAUAAAUAU
AAAGUGGUGAAGAUCGAGCCUCUGGGCGUGGCCCC
AACUAGGUGUAAAAGAAGGGUCGUCGGCUCCCACA
GCGGCAGCGGCGGCUCCGGCUCUGGCGGCCACGCG
GCUGUCGGCAUCGGCGCCGUGAGCCUGGGCUUUCU
GGGCGCCGCCGGCUCCACUAUGGGCGCAGCCUCUA
UGACCCUGACUGUCCAGGCUAGAAAUCUGCUGUCU
GGAAUCGUGCAGCAGCAGUCUAACCUGCUGAGGGC
ACCUGAGCCACAACAGCACCUGCUGAAGGAUACAC
AUUGGGGCAUCAAGCAGUUACAAGCCAGGGUGCUG
GCCGUGGAACACUACCUGCGCGAUCAGCAAUUACU
GGGCAUUUGGGGAUGCUCUGGCAAGCUGAUUUGUU
GCACCAAUGUGCCCUGGAACUCCUCUUGGAGCAAC
AGAAACCUGUCCGAAAUCUGGGAUAACAUGACAUG
GCUGCAGUGGGACAAGGAAAUUUCCAAUUAUACCC
AGAUCAUCUAUGGACUGCUGGAAGAAAGUCAGAAU
CAGCAGGAGAAGAAUGAACAGGAUCUGCUGGCACU
GGAUGGCGGCGCCGAAAACCUGUGGGUCACCGUGU
AUUAUGGAGUGCCAGUGUGGAAGGACGCCGAGACC
ACACUGUUUUGUGCCUCUGAUGCCAAGGCCUACGA
GACCGAGAAGCACAACGUGUGGGCCACCCACGCCU
GCGUGCCCACAGACCCAAAUCCUCAGGAGAUCCAC
CUGGAGAACGUGACCGAGGAGUUUAACAUGUGGAA
GAACAAUAUGGUGGAGCAGAUGCACGAGGAUAUCA
UCUCUCUGUGGGAUCAGUCUCUGAAGCCAUGUGUG
AAGCUGACCCCACUGUGCGUGACCCUGCAGUGUAC
AAAUGUGACAAACAACAUCACAGAUGACAUGAGAG
GCGAGCUGAAGAACUGUUCCUUCAAUAUGACCACC
GAGCUGAGAGACAAGAAGCAGAAGGUGUAUUCUCU
GUUUUACCGGCUGGACGUGGUGCAGAUCAACGAGA
AUCAGGGCAAUCGGUCUAACAACUCCAAUAAGGAG
UAUAGACUGAUCAACUGCAACACCUCUGCCAUCAC
CCAGGCCUGUCCUAAGGUGUCCUUUGAGCCAAUCC
CAAUCCACUAUUGCGCCCCUGCCGGCUUUGCCAUC
CUGAAGUGCAAGGACAAGAAGUUUAACGGCACAGG
CCCCUGCCCAUCCGUGAGCACAGUGCAGUGUACCC
ACGGCAUCAAGCCUGUGGUGUCCACCCAGCUGCUG
CUGAACGGCUCCCUGGCCGAGGAGGAGGUAAUCAU
CAGGUCUGAGAACAUCACAAAUAACGCCAAGAACA
UCCUGGUGCAGCUGAACACCCCAGUGCAGAUCAAC
UGUACCCGGCCUAACAAUAAUACCGUGAAGUCUAU
CCGGAUCGGCCCAGGCCAGGCCUUCUACUAUACCG
GCGAUAUCAUCGGCGAUAUCAGACAGGCCCACUGC
AACGUGUCCAAGGCCACAUGGAACGAGACACUGGG
CAAGGUGGUGAAGCAGCUGCGGAAGCACUUUGGCA
AUAACACCAUCAUCAGAUUCGCCCAGUCUUCCGGC
GGCGACCUGGAGGUGACAACCCACUCCUUCAAUUG
CGGCGGCGAGUUCUUUUACUGUAAUACAAGCGGCC
UGUUUAAUAGCACCUGGAUCUCUAACACCUCCGUG
CAGGGCUCCAACAGCACAGGCUCUAAUGAUUCCAU
CACCCUGCCUUGCCGGAUCAAGCAGAUCAUCAAUA
UGUGGCAGAGAAUCGGCCAGGCCAUGUAUGCCCCU
CCAAUCCAGGGCGUGAUCCGCUGCGUGUCCAACAU
CACAGGCCUGAUCCUGACAAGAGAUGGCGGCUCCA
CCAACAGCACCACAGAGACCUUCAGACCCGGCGGC
GGCGACAUGCGCGACAACUGGAGAUCCGAGCUGUA
UAAGUACAAGGUGGUGAAGAUCGAGCCCCUGGGCG
UGGCCCCAACCCGGUGUAAGCGCAGAGUGGUGGGC
AGCCACAGCGGCAGCGGCGGCAGCGGCUCCGGCGG
CCACGCCGCCGUGGGCAUCGGCGCCGUGUCCCUGG
GCUUCCUGGGCGCCGCCGGCUCCACCAUGGGCGCC
GCCUCCAUGACACUGACAGUGCAGGCCAGAAAUCU
GCUGUCCGGCAUCGUGCAGCAGCAGUCCAAUCUGC
UGCGGGCCCCUGAGCCACAGCAGCACCUGCUGAAG
GAUACCCACUGGGGCAUCAAGCAGCUGCAGGCCCG
GGUGCUGGCCGUGGAGCACUACCUGAGGGAUCAGC
AGCUGCUGGGCAUCUGGGGCUGUUCCGGCAAGCUG
AUCUGCUGUACAAACGUGCCCUGGAACAGCUCCUG
GUCCAAUAGGAACCUGUCCGAGAUCUGGGAUAACA
UGACCUGGCUGCAGUGGGAUAAGGAGAUCAGCAAC
UACACACAGAUCAUCUACGGCCUGCUGGAGGAGAG
CCAGAAUCAGCAGGAGAAGAACGAGCAGGACCUGC
UGGCCCUGGAUGGAGGAGGAAGCGGGGGAAGCGGG
GGAAGCGGAGGAAGCGGGGGAAGCGGGGGAAGCAA
CGCCGUGGGCCAGGACACCCAGGAAGUGAUCGUGG
UGCCCCACAGCCUGCCUUUCAAGGUGGUGGUCAUC
UCCGCCAUCCUGGCCCUGGUCGUGCUGACUAUUAU
UUCCCUGAUUAUCCUGAUUAUGCUGUGGCAGAAGA
AGCCCAGA
TRO11_AY835445_MD39_L14G8-RNA
(SEQ ID NO: 247)
AUGGAUUGGACUUGGAUUCUGUUUCUGGUCGCUGC
UGCUACUCGGGUGCAUUCUCAGGGCCAGCUGUGGG
UCACUGUCUACUACGGCGUGCCAGUGUGGAAGGAC
GCCUCUACCACACUGUUUUGCGCCAGCGACGCCAA
GGCCUACGAUACAGAGGUGCACAACGUGUGGGCAA
CACACGCAUGCGUGCCAACCGAUCCAAAUCCCCAG
GAGGUGGUGCUGGGCAACGUGACCGAGAACUUCAA
UAUGUGGAAGAACAAUAUGGUGGACCAGAUGCACG
AGGAUAUCAUCUCUCUGUGGGACCAGAGCCUGAAG
CCCUGCGUGAAGCUGACCCCUCUGUGCGUGACACU
GAAUUGUACCGAUAACAUCACCAACACAAAUACCA
ACAGCUCCAAGAACUCUAGCACACACUCCUAUAAC
AAUUCUCUGGAGGGCGAGAUGAAGAAUUGUUCCUU
UAACAUCACCGCCGGCAUCCGGGACAAGGUGAAGA
AGGAGUACGCCCUGUUCUAUAAGCUGGAUGUGGUG
CCCAUCGAGGAGGACAAGGAUACAAAUAAGACCAC
AUACCGGCUGCGCAGCUGCAACACAUCCGUGAUCA
CCCAGGCCUGUCCUAAGGUGACCUUUGAGCCUAUC
CCAAUCCACUAUUGCGCCCCAGCCGGCUUCGCCAU
CCUGAAGUGUAAUGACAAGAAGUUUAACGGCACAG
GCCCCUGCACCAACGUGUCUACAGUGCAGUGUACC
CACGGCAUCAGGCCUGUGGUGUCCACCCAGCUGCU
GCUGAAUGGCUCUCUGGCCGAGGAGGAAGUGAUCA
UCAGAAGCGAGAACUUUACAAACAAUGCCAAGACC
AUCAUCGUGCAGCUGAAUGAGUCUAUCGCCAUCAA
CUGCACAAGGCCAAACAAUAACACCGUGAGAAGCA
UCCACAUCGGACCAGGAAGGGCCUUCUACUAUACC
GGCGACAUCAUCGGCGAUAUCAGGCAGGCCCACUG
UAAUAUCUCCAGAACAGAGUGGAACUCUACCCUGC
GGCAGAUCGUGACAAAGCUGCGCGAGCAGCUGGGC
GACCCUAACAAGACCAUCAUCUUCGCCCAGUCCUC
UGGCGGCGAUACAGAGAUCACCAUGCACUCCUUUA
AUUGCGGCGGCGAGUUCUUUUACUGUAACACCACA
AAGCUGUUCAAUUCUACCUGGAACGGCAAUAACAC
CACAGAGUCCGACUCUACAGGCGAGAAUAUCACCC
UGCCAUGCCGGAUCAAGCAGAUCAUCAACCUGUGG
CAGGAAGUGGGCAAGGCCAUGUAUGCCCCUCCCAU
CAAGGGCCAGAUCUCCUGUAGCUCCAACAUCACAG
GCCUGCUGCUGACCCGCGACGGCGGAAAUAACAAU
UCUAGCGGACCAGAGACAUUCAGGCCUGGCGGCGG
CAAUAUGAAGGAUAACUGGAGAAGCGAGCUGUACA
AGUAUAAAGUGAUCAAGAUCGAGCCUCUGGGAGUG
GCACCAACCAGGUGCAAGAGGAGAGUGGUGGGCAG
CCACUCCGGCUCUGGCGGCAGCGGCUCCGGCGGCC
ACGCAGCAGUGGGCACACUGGGCGCCAUGAGCCUG
GGCUUCCUGGGAGCAGCAGGCAGCACCAUGGGAGC
AGCAUCCGUGACACUGACCGUGCAGGCAAGGCUGC
UGCUGUCCGGCAUCGUGCAGCAGCAGAACAAUCUG
CUGAGGGCACCAGAGCCUCAGCAGCACAUGCUGCA
GGACACACACUGGGGCAUCAAGCAGCUGCAGGCCC
GGGUGCUGGCAGUGGAGCACUACCUGCGCGAUCAG
CAGCUGCUGGGCAUCUGGGGCUGUAGCGGCAAGCU
GAUCUGCUGUACCAAUGUGCCUUGGAACGCCUCUU
GGAGCAAUAAGAGCCUGAACAAUAUCUGGGAGAAU
AUGACAUGGAUGAACUGGUCCAGAGAGAUCGACAA
CUACACCGAUCUGAUCUAUAUCCUGCUGGAGAAGU
CACAGAUUCAGCAGGAGAAGAACAAUCAGAGCCUG
CUGGAACUGGAU
X2278_FJ817366_MD39_L14G8-RNA
(SEQ ID NO: 248)
AUGGACUGGACCUGGAUUCUGUUCCUGGUCGCCGC
UGCUACAAGAGUGCAUUCUACAAAUAACCUGUGGG
UGACUGUCUACUAUGGAGUGCCCGUGUGGAAGGAG
GCCACCACAACCCUGUUCUGCGCCAGCGAGGCCAA
GGCCUACGACACAGAGGUGCACAACAUCUGGGCCA
CCCACGCCUGCGUGCCUACAGAUCCAAACCCCCAG
GAGAUGGAGCUGAAGAAUGUGACCGAGAACUUCAA
CAUGUGGAAGAACAAUAUGGUGGAGCAGAUGCACG
AGGACAUCAUCAGCCUGUGGGAUCAGUCCCUGAAG
CCCUGCGUGAAGCUGACACCUCUGUGCGUGACCCU
GGAUUGUACAAAUAUCAACAGCACAAACUCCACCA
ACAAUACAAGCUCCAAUUCUAAGAUGGAGGAGACA
AUCGGCGUGAUCAAGAAUUGUAGCUUCAACGUGAC
AACCAAUAUCCGGGACAAGGUGAAGAAGGAGAACG
CCCUGUUUUACUCUCUGGAUCUGGUGAGCAUCGGC
AAUUCUAACACCAGCUAUCGCCUGAUCUCCUGCAA
UACCUCUAUCAUCACACAGGCCUGUCCAAAGGUGA
GCUUCGACCCUAUCCCAAUCCACUACUGCGCACCA
GCAGGAUUCGCAAUCCUGAAGUGUAGGGAUAAGAA
GUUUAACGGCACCGGCCCUUGCAGAAACGUGAGCA
GCGUGCAGUGUACACACGGCAUCAGGCCAGUGGUG
AGCACCCAGCUGCUGCUGAACGGCUCCCUGGCAGA
GGAGGAGAUCAUCAUCAGAUCCGCCAACCUGACCG
ACAAUGCCAAGACAAUCAUCAUCCAGCUGAACGAG
ACAAUCCAGAUCAAUUGCACAAGGCCCAACAAUAA
CACCGUGAGAAGCAUCCCAAUCGGCCCCGGCCGGA
CCUUUUACUAUACAGGCGACAUCAUCGGCGAUAUC
CGCAAGGCCUACUGUAACAUCUCCGCCACCAAGUG
GAAUAACACACUGCGGCAGAUCGCCGAGAAGCUGC
GCGAGAAGUUCAACAAGACAAUCAUCUUUGCCCAG
UCCUCUGGCGGCGAUCCAGAGGUGGUGAGGCACAC
CUUCAAUUGCGGCGGCGAGUUCUUUUACUGUAACA
GCUCCCAGCUGUUUAAUAGCACAUGGUAUUCCAAC
GGCACCUCUAAUGGCGGCCUGAAUAACAGCGCCAA
CAUCACCCUGCCCUGCAGAAUCAAGCAGAUCAUCA
AUCUGUGGCAGGAAGUGGGCAAGGCCAUGUAUGCC
CCUCCCAUCAAGGGCGUGAUCAACUGUCUGUCCAA
UAUCACCGGCAUCAUCCUGACAAGGGACGGCGGCG
AGAAUAACGGCACAACCGAGACAUUCAGACCCGGC
GGCGGCGACAUGAGGGAUAACUGGCGCUCUGAGCU
GUACAAGUAUAAGGUGGUGAAGAUCGAGCCUCUGG
GCAUCGCCCCAACCAAGUGCAAGAGGAGAGUGGUG
GGCUCUCACAGCGGCUCCGGCGGCUCUGGCAGCGG
CGGCCACGCAGCAGUGGGCCUGGGAGCCGUGUCUC
UGGGCUUUCUGGGCCUGGCAGGCUCCACAAUGGGA
GCAGCCUCUGUGACACUGACCGUGCAGGCAAGGCU
GCUGCUGAGCGGCAUCGUGCAGCAGCAGAAUAACC
UGCUGAGGGCACCAGAGCCUCAGCAGCAGCUGCUG
CAGGACACCCACUGGGGCAUCAAGCAGCUGCAGGC
CCGGGUGCUGGCCCUGGAGCACUACCUGAAGGAUC
AGCAGCUGCUGGGCAUCUGGGGCUGUUCCGGCAAG
CUGAUCUGCUGUACAACCGUGCCAUGGAACGCCUC
CUGGUCUAACAAGUCCUAUAAUCAGAUCUGGAAUA
ACAUGACAUGGAUGAACUGGAGCAGGGAGAUCGAC
AAUUACACCAACCUGAUCUAUAAUCUGAUUGAAGA
GUCACAGUCACAGCAGGAAAAGAACAACCUGAGCC
UGCUGCAGCUGGAC
398F1_HM215312_MD39_L14G8-nucleic acid
(SEQ ID NO: 249)
AUGGACUGGACUUGGAUUCUGUUUCUGGUCGCAGC
CGCAACUAGAGUGCAUAGCAUGGGCAACCUGUGGG
UCACCGUGUAUUACGGGGUGCCAGUGUGGAAGGAC
GCCGAGACUACGCUGUUCUGCGCCUCCGAUGCCAA
GGCCUACCACACAGAGGUGCACAACGUGUGGGCAA
CCCACGCAUGCGUGCCAACAGACCCAAAUCCCCAG
GAGAUCAACCUGGAGAAUGUGACCGAGGAGUUUAA
CAUGUGGAAGAAUAAGAUGGUGGAGCAGAUGCACG
AGGACAUCAUCUCCCUGUGGGAUCAGUCUCUGAAG
CCUUGCGUGCAGCUGACCCCACUGUGCGUGACACU
GGACUGUCAGUACAACGUGACCAACAUCAAUAGCA
CAUCCGAUAUGGCCAGGGAGAUCAACAAUUGUAGC
UAUAAUAUCACCACAGAGCUGCGGGAUCGCGAGCA
GAAAGUGUACAGCCUGUUCUAUAGGUCCGACAUCG
UGCAGAUGAACUCCGAUAAUAGCUCCAAGUACAGA
CUGAUCAACUGCAAUACCUCUGCCAUCAAGCAGGC
CUGUCCAAAGGUGACAUUUGAGCCUAUCCCAAUCC
ACUAUUGCGCACCAGCAGGAUUCGCAAUCCUGAAG
UGUAAGGACAAGGAGUUUAACGGCACCGGCCCUUG
CAAGAACGUGAGCACCGUGCAGUGUACACACGGCA
UCAAGCCAGUGGUGAGCACACAGCUGCUGCUGAAC
GGCUCCCUGGCCGAGGAGAAAGUGAUCAUCCGGUC
UGAGAAUAUCACCGAUAACGCCAAGAAUAUCAUCG
UGCAGCUGAAGGAGCCCGUGAAGAUCAACUGCACC
CGGCCUAACAAUAACACAGUGAAGUCCGUGCGCAU
CGGCCCUGGCCAGACCUUCUACUAUACAGGCGAGA
UCAUCGGCGACAUCCGCCAGGCCCACUGUAACGUG
UCUAAGGCCCACUGGGAGAACACCCUGCAGGAGGU
GGCCAAUCAGCUGAAGCUGAUGAUCCACAGCAACA
AGACAAUCAUCUUCGCCAAUUCUAGCGGCGGCGAU
CUGGAGAUCACCACACACUCUUUUAACUGCGGCGG
CGAGUUCUUUUACUGUUAUACCAGCGGCCUGUUCA
ACUACACCUUCAACGACACCAGCACAAACUCCACC
GAGUCUAAGAGCAAUGAUACCAUCACACUGCAGUG
CAGGAUCAAGCAGAUCAUCAACAUGUGGCAGAGAG
CAGGACAGGCCGUGUAUGCCCCUCCCAUCCCCGGC
AUCAUCCGGUGUGAGAGCAAUAUCACCGGCCUGAU
CCUGACACGCGACGGCGGAAAUAACAAUUCCAACA
CCAAUGAGACAUUCAGGCCCGGCGGCGGCGACAUG
AGGGAUAACUGGAGAUCUGAGCUGUACAGAUAUAA
GGUGGUGAAGAUCGAGCCAAUCGGCGUGGCCCCCA
CCACAUGCAAGAGGAGAGUGGUGGGCUCCCACUCU
GGCAGCGGCGGCUCCGGCUCUGGCGGCCACGCAGC
CGUGGGCAUCGGAGCCGUGAGCCUGGGCUUUCUGG
GAGCAGCAGGCUCUACCAUGGGAGCAGCCAGCAUC
ACCCUGACAGUGCAGGCAAGGCAGCUGCUGUCCGG
AAUCGUGCAGCAGCAGUCUAACCUGCUGAGGGCAC
CAGAGCCUCAGCAGCACCUGCUGAAGGACACCCAC
UGGGGCAUCAAGCAGCUGAAGGCCAGGGUGCUGGC
CGUGGAGCACUACCUGAAGGAUCAGCAGCUGCUGG
GCAUCUGGGGCUGUAGCGGCAAGCUGAUCUGCUGU
ACCAACGUGCCCUGGAAUUCCUCUUGGUCUAACAA
GAGCCUGGGCGAGAUCUGGGACAACAUGACCUGGC
UGAAUUGGUCCAAGGAGAUCGAGAAUUACACACAG
AUCAUCUAUGAGCUGAUUGAAGAGUCACAGAACCA
GCAGGAGAAAAACAACCAGAGCCUGCUGGCACUGG
AU
246F3_HM215279_MD39_L14G8-nucleic acid
(SEQ ID NO: 250)
AUGGACUGGACUUGGAUUCUGUUUCUGGUCGCAGC
CGCUACUCGGGUGCACUCUAUGCAGGACCUGUGGG
UGACCGUCUAUUAUGGGGUGCCAGUGUGGAAGGAC
GCCAAGACCACACUGUUCUGCGCCUCCGAUGCCAA
GGCCUACGAGAAGGAGGUGCACAACGUGUGGGCAA
CCCACGCAUGCGUGCCAACAGACCCAAACCCCCAG
GAGAUCGUGAUGGCCAAUGUGACCGAGGAGUUUAA
CAUGUGGAAGAACAAUAUGGUGGAGCAGAUGCACG
AGGACAUCAUCUCUCUGUGGGAUCAGAGCCUGAAG
CCUUGCGUGAAGCUGACCCCACUGUGCGUGACACU
GGACUGUAAGGAUUACAACUAUUCCAUCACCAACA
AUUCUACAGGCAUGGAGGGCGAGAUCAAGAAUUGU
UCUUAUAACAUCACCACAGAGCUGCGCGACAAGAG
GCAGAAAGUGUACAGCCUGUUCUAUCGCCUGGAUG
UGGUGCAGAUCAAUGACUCUAACGAUCGCAACAAU
AGCCAGUACAGGCUGAUCAAUUGCAACACCACAAC
CAUGACCCAGGCCUGUCCUAAGGUGACAUUUGACC
CUAUCCCAAUCCACUAUUGCGCCCCAGCCGGCUUC
GCCAUCCUGAAGUGUAACAAUAAGACCUUUAAUGG
CAAGGGCCCCUGCAACAAUGUGAGCUCCGUGCAGU
GUACCCACGGCAUCAAGCCUGUGGUGUCUACACAG
CUGCUGCUGAACGGCAGCCUGGCCGAGAAGGAGAU
CAUCAUCAGGAGCGAGAAUCUGACCGACAACGUGA
AGACAAUCAUCGUGCACCUGAAUGAGAGCGUGGAG
AUCAACUGCACCAGACCAAACAAUAACACAGUGAA
GUCCGUGCGGAUCGGACCAGGACAGACCUUCUACU
AUACAGGCGAUAUCAUCGGCAAUAUCCGCCAGGCC
CACUGUACCGUGAAUAAGACAGAGUGGAACACAGC
CCUGACCAGGGUGAGCAAGAAGCUGAAGGAGUACU
UCCCCAACAAGACCAUCGCCUUUCAGCCUUCUAGC
GGCGGCGACCUGGAGAUCACAACCUUCUCCUUUAA
UUGCAGAGGCGAGUUCUUUUAUUGUAACACAUCCG
AUCUGUUCAAUGGCACCUUUAACGAGACAUCUGGC
CAGUUCAAUUCCACCUUUAACUCUACACUGCAGUG
CCGGAUCAAGCAGAUCAUCAAUAUGUGGCAGGAAG
UGGGACAGGCAAUGUACGCCCCUCCCAUCGCAGGC
AGCAUCACCUGUAUCUCCAACAUCACCGGCCUGAU
CCUGACACGCGACGGCGGAAAUACAAACUCCACCA
AGGAGACAUUCAGGCCUGGCGGCGGCAAUAUGAGA
GAUAACUGGCGGUCUGAGCUGUACAAGUAUAAGGU
GGUGAAGAUCGAGCCACUGGGAGUGGCACCAACCA
AGUGCAGGAGACGGGUGGUGGGCAGCCACUCCGGC
UCUGGCGGCAGCGGCUCCGGCGGCCACGCAGCAGU
GGGCAUCGGCGCCGUGUCUAUCGGCUUUCUGGGAG
CAGCAGGCUCCACCAUGGGAGCAGCCUCUAUCACA
CUGACCGUGCAGGCCAGACAGCUGCUGAGCGGCAU
CGUGCAGCAGCAGUCCAACCUGCUGAGGGCACCAG
AGCCUCAGCAGCACCUGCUGAAGGACACCCACUGG
GGCAUCAAGCAGCUGCAGGCCAGGGUGCUGGCAGU
GGAGCACUACCUGAAGGAUCAGCAGCUGCUGGGCA
UCUGGGGCUGUAGCGGCAAGCUGAUCUGCUGUACA
AAUGUGCCCUGGAACUCCUCUUGGUCUAACAAGAG
CCAGGACGAGAUCUGGGAUAAUAUGACCUGGCUGA
ACUGGAGCAAGGAGAUCUCCAAUUACACACAGAUC
AUCUAUAACCUGAUUGAAGAAUCACAGACUCAGCA
GGAACUGAAUAAUAGGUCACUGCUGGCACUGGAU
CE0217_FJ443575_MD39_L14G8
(SEQ ID NO: 251)
AUGGACUGGACUUGGAUUCUGUUUCUGGUCGCCGC
CGCAACUCGCGUGCAUUCAGCAAAAGAUAUGUGGG
UCACCGUCUAUUAUGGAGUGCCCGUGUGGCGGGAG
GCCAAGACCACACUGUUUUGCGCAAGCGACGCAAA
GGCAUACGAGAGGGAGGUGCACAACGUGUGGGCCA
CACACGCCUGCGUGCCAACCGAUCCAAAUCCCCAG
GAGAGAGUGCUGGAGAACGUGACCGAGAAUUUCAA
CAUGUGGAAGAACAAUAUGGUGGACCAGAUGCACG
AGGAUAUCAUCUCUCUGUGGGACGAGAGCCUGAAG
CCCUGCAUCAAGCUGACACCUCUGUGCGUGACCCU
GAAUUGUGGCAACGCCAUCGUGAAUGAGUCCACCA
UCGAGGGCAUGAAGAAUUGUUCUUUUAACGUGACC
ACAGAGCUGAAGGACAAGAAGAAGAAGGAGUACGC
CCUGUUCUAUAAGCUGGAUGUGGUGCCCCUGAACG
GCGAGAACAACAACUCUAACAGCAAGAACUUUAGC
GAGUACAGGCUGAUCAAUUGCAACACCUCCACAAU
CACCCAGGCCUGUCCCAAGGUGUCUUUCGAUCCUA
UCCCAAUCCACUAUUGCGCCCCUGCCGGCUUCGCC
AUCCUGAAGUGUAAUAACGAGACAUUCAACGGCAC
CGGCCCAUGCAAUAACGUGUCCACAGUGCAGUGUA
CCCACGGCAUCAAGCCCGUGGUGUCUACACAGCUG
CUGCUGAAUGGCAGCCUGGCCGAGAAGGAGAUCAU
CAUCAGGUCUGAGAACCUGACCAAUAACGCCAAGA
UCAUCAUCGUGCACCUGAAUAACCCAGUGAAGAUC
AUCUGCACAAGGCCCGGCAAUAACACCGUGAAGAG
CAUGAGAAUCGGCCCUGGCCAGACAUUCUACUAUA
CCGGCGACAUCAUCGGCGAUAUCAGGAGAGCCUAC
UGUAACAUCUCUGAGAAGACAUGGUAUGACACCCU
GAAGAAUGUGAGCGAUAAGUUCCAGGAGCACUUUC
CUAACGCCUCCAUCGAGUUCAAGCCAUCUGCCGGC
GGCGACCUGGAGAUCACCACACACUCCUUUAAUUG
CAGGGGCGAGUUCUUUUACUGUGAUACAAGCGAGC
UGUUCAAUGGCACAUACAAUAACUCCACCUAUAAC
AGCUCCAAUAACAUCACCCUGCAGUGCAAGAUCAA
GCAGAUCAUCAACAUGUGGCAGGGCGUGGGCAGAG
CCAUGUAUGCCCCUCCCAUCGCCGGCAAUAUCACC
UGUGAGAGCAACAUCACAGGCCUGCUGCUGACCCG
GGACGGCGGAAAUAACAAGUCCACACCAGAGACAU
UCAGGCCCGGCGGCGGCGACAUGAGGGAUAACUGG
AGAAGCGAGCUGUACAAGUAUAAGGUGGUGGAGAU
CAAGCCUCUGGGCAUCGCCCCAACAAAGUGCAAGA
GGAGGGUGGUGGGCUCCCACUCUGGCAGCGGCGGC
UCCGGCUCUGGCGGCCACGCAGCCGUGGGCAUGGG
CGCCGUGUCUCUGGGCUUCCUGGGAGCAGCAGGCA
GCACCAUGGGAGCAGCAUCCCUGACACUGACCGUG
CAGGCAAGGCAGCUGCUGAGCGGCAUCGUGCAGCA
GCAGAAUAACCUGCUGAGAGCCCCCGAGCCUCAGC
AGCACAUGCUGCAGGACACACACUGGGGCAUCAAG
CAGCUGCAGGCCCGGGUGCUGGCAAUCGAGCACUA
CCUGACAGAUCAGCAGCUGCUGGGCAUCUGGGGCU
GUUCCGGCAAGCUGAUCUGCUGUACCAAUGUGCCC
UGGAAUAACAGCUGGUCCAACAAGUCCUAUGAGGA
UAUCUGGGGCCGGAAUAUGACCUGGAUGAACUGGA
GCAGGGAGAUCAACAACUACACAAACACCAUCUAU
CGCCUGCUGGAAAAGUCACAGAAUCAGCAGGAGAA
GAAUAAUAAGUCACUGCUGGAACUGGAC
CE1176_FJ444437_MD39_L14G8-RNA
(SEQ ID NO: 252)
AUGGAUUGGACUUGGAUUCUGUUUCUGGUCGCCGC
CGCUACUCGCGUGCAUUCAGUGGGCAACCUGUGGG
UCACCGUCUACUAUGGGGUGCCCGUGUGGAAGGAG
GCCAAGACCACACUGUUCUGCGCCUCCGACGCCAA
GGCCUACGAGAAGGAGGUGCACAACGUGUGGGCCA
CACACGCCUGCGUGCCUACCGAUCCAAAUCCCCAG
GAGAUGGUGCUGGAGAACGUGACAGAGAACUUUAA
UAUGUGGAAGAACGACAUGGUGGAUCAGAUGCACG
AGGACGUGAUCUCUCUGUGGGAUCAGAGCCUGAAG
CCUUGCGUGAAGCUGACCCCACUGUGCGUGACCCU
GACAUGUACCAAUACCACAGUGUCCAACGGCAGCU
CCAACUCUAAUGCCAACUUCGAGGAGAUGAAGAAU
UGUUCUUUUAACGCCACCACAGAGAUCAAGGACAA
GAAGAAGAACGAGUACGCCCUGUUCUAUAAGCUGG
AUAUCGUGCCCCUGAACAAUUCUAGCGGCAAGUAU
AGGCUGAUCAAUUGCAACACAAGCGCCAUCGCCCA
GGCCUGUCCAAAGGUGACCUUCGAGCCUAUCCCAA
UCCACUACUGCGCCCCCGCCGGCUAUGCCAUCCUG
AAGUGUAACAACAAGACCUUCAACGGCACCGGCCC
UUGCAACAACGUGAGCACAGUGCAGUGUACCCACG
GCAUCAAGCCAGUGGUGAGCACCCAGCUGCUGCUG
AACGGCUCCCUGGCAGAGAAGGAGAUCAUCAUCCG
GAGCGAGAAUCUGACAAACAAUGCCAAGACCAUCA
UCAUCCACCUGAACGAGUCCGUGGGCAUCGUGUGC
ACACGGCCCAGCAACAAUACCGUGAAGUCCAUCCG
CAUCGGCCCUGGCCAGACCUUCUACUAUACCGGCG
ACAUCAUCGGCGAUAUCCGCCAGGCCCACUGUAAU
GUGAGCAAGCAGAAUUGGAACAGGACACUGCAGCA
AGUGGGCAGAAAGCUGGCCGAGCACUUCCCAAAUA
GGAACAUCACCUUUGCCCACUCCUCUGGCGGCGAC
CUGGAGAUCACCACACACUCCUUCAACUGCAGAGG
CGAGUUCUUUUACUGUAAUACAUCUGGCCUGUUUA
ACGGCACCUACCACCCCAAUGGCACAUAUAACGAG
ACAGCCGUGAAUAGCUCCGAUACAAUCACCCUGCA
GUGCAGGAUCAAGCAGAUCAUCAACAUGUGGCAGG
AAGUGGGCAGAGCCAUGUAUGCCCCUCCCAUCGCC
GGCAAUAUCACCUGUAACAGCACAAUCACCGGCCU
GCUGCUGACACGGGACGGCGGCAUCAACCAGACCG
GAGAGGAGAUCUUCCGCCCCGGCGGCGGCGACAUG
CGGGAUAAUUGGCGCAACGAGCUGUACAAGUAUAA
GGUGGUGGAGAUCAAGCCACUGGGCAUCGCCCCCA
CAAAGUGCAAGAGGAGAGUGGUGGGCUCCCACUCU
GGCAGCGGCGGCUCCGGCUCUGGCGGCCACGCAGC
CGUGGGCAUCGGAGCCGUGUCCCUGGGCUUUCUGG
GAGCAGCAGGCUCUACCAUGGGAGCAGCCAGCAUC
ACACUGACCGUGCAGGCAAGGCAGCUGCUGUCCGG
CAUCGUGCAGCAGCAGUCUAACCUGCUGAGAGCCC
CCGAGCCUCAGCAGCACAUGCUGCAGGACACCCAC
UGGGGCAUCAAGCAGCUGCAGGCCAGGGUGCUGGC
CAUCGAGCACUACCUGAAGGAUCAGCAGCUGCUGG
GCAUCUGGGGCUGUUCUGGCAAGCUGAUCUGCUGU
ACAAAUGUGCCAUGGAACUCUAGCUGGAGCAACCG
GUCCCAGGAGGACAUCUGGAACAAUAUGACCUGGA
UGAAUUGGAGCAGGGAGAUCGAUAACUACACACAC
ACCAUCUAUAGCCUGCUGGAGGAGUCACAGAUUCA
GCAGGAGAAAAAUAAUAAGUCACUGCUGGCACUGG
AC
25710_EF117271_MD39_L14G8-RNA
(SEQ ID NO: 253)
AUGGACUGGACUUGGAUUCUGUUCCUGGUCGCCGC
CGCUACUCGCGUGCAUUCUGGGGGCAACCUGUGGG
UCACCGUGUAUUAUGGAGUGCCCGUGUGGAAGGAG
GCCACCACAACCCUGUUCUGCGCCAGCGACGCCAA
GGCCUACGAUAAGGAGGUGCACAACGUGUGGGCAA
CCCACGCAUGCGUGCCAACAGACCCAAACCCCCAG
GAGAUGGUGCUGGGCAAUGUGACCGAGAACUUUAA
UAUGUGGAAGAACGAGAUGGUGAAUCAGAUGCACG
AGGACGUGAUCUCCCUGUGGGAUCAGUCUCUGAAG
CCUUGCGUGAAGCUGACCCCACUGUGCGUGACACU
GGAGUGUUCCAACGUGACCUAUAAUGAGUCUAUGA
AGGAGGUGAAGAACUGUUCCUUCAAUCUGACAACC
GAGCUGAGGGAUAAGAAGCAGAAGGUGCACGCCCU
GUUUUACAGACUGGACAUCGUGCCCCUGAACGAUA
CCGAGAAGAAGAAUAGCUCCCGGCCUUAUCGCCUG
AUCAACUGCAAUACAAGCGCCAUCACCCAGGCCUG
UCCUAAGGUGACCUUCGACCCUAUCCCAAUCCACU
ACUGCACACCAGCCGGCUAUGCCAUCCUGAAGUGU
AACGAUAAGAAGUUUAAUGGCACCGGCCCAUGCCA
CAAGGUGUCCACAGUGCAGUGUACCCACGGCAUCA
AGCCCGUGGUGUCUACACAGCUGCUGCUGAACGGC
AGCCUGGCAGAGGGCGAGAUCAUCAUCAGGAGCGA
GAACCUGACCAACAAUGCCAAGACAAUCAUCGUGC
ACCUGAAUCAGUCCGUGGAGAUCGUGUGCGCCCGG
CCAAGCAACAAUACAGUGACCUCCAUCAGGAUCGG
ACCAGGACAGACAUUCUACUAUACCGGCGCCAUCA
CAGGCGACAUCAGGCAGGCCCACUGUAACAUCAGC
AAGGAUAAGUGGAAUGAGACACUGCAGAGAGUGGG
CGAGAAGCUGGCCGAGCACUUCCCCAACAAGACAA
UCAAGUUUGCCUCUAGCUCCGGCGGCGACCUGGAG
AUCACAACCCACUCCUUUAACUGCAGGGGCGAGUU
CUUUUACUGUAAUACCUCUGGCCUGUUCAACGGCA
CCUUUAAUGGCACAUACGUGAGCCCCAACAGCACC
GAUUCCAAUUCUAGCUCCAUCAUCACAAUCCCUUG
CCGGAUCAAGCAGAUCAUCAAUAUGUGGCAGGAAG
UGGGAAGGGCAAUGUACGCCCCUCCCAUCGCCGGC
AACAUCACCUGUAAGUCCAAUAUCACAGGCCUGCU
GCUGGUGAGGGACGGCGGAACCGGCUCUGAGAGCA
ACAAGACAGAGAUCUUCAGACCCGGCGGCGGCGAC
AUGAGGGAUAAUUGGAGAUCUGAGCUGUACAAGUA
UAAGGUGGUGGAGAUCAAGCCACUGGGCGUGGCCC
CCACCAAGUGCAAGAGGAGAGUGGUGGGCUCCCAC
UCUGGCAGCGGCGGCUCCGGCUCUGGCGGCCACGC
AGCCGUGGGCAUCGGAGCCGUGUCCCUGGGCUUUC
UGGGAGCAGCAGGCUCUACAAUGGGAGCAGCCAGC
AUCACACUGACCGUGCAGGCAAGGCAGCUGCUGAG
CGGCAUCGUGCAGCAGCAGUCCAACCUGCUGAGGG
CACCAGAGCCUCAGCAGCACCUGCUGCAGGACACC
CACUGGGGCAUCAAGCAGCUGCAGACACGGGUGCU
GGCCAUCGAGCACUACCUGAAGGAUCAGCAGCUGC
UGGGCAUCUGGGGCUGUUCUGGCAAGCUGAUCUGC
UGUACCGCCGUGCCCUGGAACUAUAGCUGGUCCAA
UCGCAGCCAGGACGAUAUCUGGGACAACAUGACAU
GGAUGAAUUGGUCUAAGGAGAUCAGCAACUACACA
AAUACCAUCUAUAAGCUGCUGGAAGAUAGUCAGAU
UCAGCAGGAAAAGAACAAUAAGUCACUGCUGGCAC
UGGAU
BJOX2000_HM215364_MD39_L14G8-RNA
(SEQ ID NO: 254)
AUGGACUGGACUUGGAUUCUGUUUCUGGUCGCAGC
AGCAACUCGGGUGCAUAGCGUCGGCAACCUGUGGG
UCACUGUCUACUACGGGGUGCCCGUGUGGAAGGAG
GCCACCACAACCCUGUUCUGCGCCAGCGACGCCAA
GGCCUACGAUACCGAGGUGCACAACGUGUGGGCAA
CCCACGCAUGCGUGCCUACAGACCCAGAUCCCCAG
GAGAUGUUCCUGGAGAACGUGACAGAGAACUUCAA
CAUGUGGAAGAACAAUAUGGUGGACCAGAUGCACG
AGGAUGUGAUCAGCCUGUGGGACCAGUCCCUGAAG
CCUUGCGUGAAGCUGACCCCACUGUGCGUGACACU
GGAGUGUAAGAAUGUGAACAGCUCCUCUAGCGACA
CCAAGAACGGCACAGAUCCUGAGAUGAAGAAUUGU
UCUUUCAACGCCACAACCGAGCUGCGGGACCGCAA
GCAGAAGGUGUACGCCCUGUUUUAUAAGCUGGAUA
UCGUGCCACUGAAUGAGAAGAACUCCUCUGAGUAU
CGGCUGAUCAAUUGCAACACAAGCACCAUCACACA
GGCCUGUCCCAAGGUGACCUUCGACCCUAUCCCAA
UCCACUACUGCACACCUGCCGGCUAUGCCAUCCUG
AAGUGUAAUGAUGAGAAGUUUAACGGCACCGGCCC
AUGCUCCAACGUGAGCACCGUGCAGUGUACACACG
GCAUCAAGCCCGUGGUGAGCACACAGCUGCUGCUG
AACGGCUCCCUGGCCGAGAAGGGCAUCAUCAUCCG
CUCCGAGAAUCUGACCAACAAUGUGAAGACAAUCA
UCGUGCACCUGAACCAGUCCGUGGAGAUCCUGUGC
AUCCGGCCAAACAAUAACACCGUGAAGUCUAUCCG
CAUCGGCCCCGGCCAGACCUUCUACUAUACAGGCG
AGAUCAUCGGCGACAUCCGGCAGGCCCACUGUAAU
AUCUCUGGCAAGGUCUGGAACGAGACACUGCAGAG
GGUGGGAGAGAAGCUGGCAGAGUACUUCCCAAACA
AGACAAUCAAGUUUGCCAGCUCCUCUGGCGGCGAU
CUGGAGAUCACAACCCACUCUUUUAAUUGCGGCGG
CGAGUUCUUUUACUGUAACACCAGCAAGCUGUUCA
AUGGCACCUUUAACGGCACAUAUAUGCCUAAUGUG
ACCGAGGGCAACAGCACAAUCUCCAUCCCAUGCCG
GAUCAAGCAGAUCAUCAAUAUGUGGCAGAAAGUGG
GCCGCGCCAUGUAUGCCCCUCCCAUCGAGGGCAAC
AUCACCUGUAAGAGCAAGAUCACAGGCCUGCUGCU
GGAGAGGGACGGCGGACCAGAGAACGAUACCGAGA
UCUUCAGACCCGGCGGCGGCGACAUGAGGAAUAAC
UGGAGAUCCGAGCUGUACAAGUAUAAGGUGGUGGA
GAUCAAGCCACUGGGAGUGGCACCAACCGAGUGCA
AGAGGAGAGUGGUGGGCUCUCACAGCGGCUCCGGC
GGCUCUGGCAGCGGCGGCCACGCCGCCGUGGGCAU
CGGAGCCGUGAGCCUGGGCUUUCUGGGAGUGGCAG
GCUCUACCAUGGGAGCAGCAAGCAUGGCACUGACA
GUGCAGGCCAGGCAGCUGCUGUCCGGCAUCGUGCA
GCAGCAGUCUAAUCUGCUGAGAGCACCAGAGCCUC
AGCAGCACCUGCUGCAGGACACCCACUGGGGCAUC
AAGCAGCUGCAGACAAGGGUGCUGGCCAUCGAGCA
CUACCUGAAGGAUCAGCAGCUGCUGGGCAUCUGGG
GCUGUUCCGGCAAGCUGAUCUGCUGUACCGCCGUG
CCUUGGAAUAGCUCCUGGUCUAACAAGAGCCAGGA
GGAGAUCUGGGAGAAUAUGACAUGGAUGAACUGGU
CCAAGGAGAUCUCUAACUACACCGAUACAAUCUAU
AGACUGCUGGAAGAUAGUCAGAAUCAGCAGGAGAG
AAAUAAUAAGUCACUGCUGGCACUGGAU
CH119_EF117261_MD39_L14G8-RNA
(SEQ ID NO: 255)
AUGGACUGGACUUGGAUUCUGUUUCUGGUCGCAGC
CGCAACUCGCGUGCAUUCCGUGGGCAACCUGUGGG
UCACCGUCUACUAUGGGGUGCCAGUGUGGAAGGAG
GCCACCACAACCCUGUUCUGCGCCUCCGACGCCAA
GGCCUACGAUACCGAGGUGCACAACGUGUGGGCAA
CACACGCAUGCGUGCCAACCGACCCAUCUCCCCAG
GAGCUGGUGCUGGAGAAUGUGACAGAGAACUUCAA
CAUGUGGAAGAAUGAGAUGGUGAACCAGAUGCACG
AGGACGUGAUCUCCCUGUGGGAUCAGUCUCUGAAG
CCUUGCGUGAAGCUGACACCACUGUGCGUGACCCU
GGAGUGUUCCAAGGUGUCUAACAAUGAGACAGACA
AGUAUAACGGCACCGAGGAGAUGAAGAAUUGUAGC
UUCAACGCAACAACCGUGGUGCGGGACCGCCAGCA
GAAGGUGUACGCCCUGUUUUAUAGGCUGGAUAUCG
UGCCCCUGACCGAGAAGAAUAGCUCCGAGAACUCU
AGCAAGUACUAUAGACUGAUCAAUUGCAACACAUC
UGCCAUCACCCAGGCCUGUCCAAAGGUGAGCUUCG
AGCCUAUCCCAAUCCACUACUGCACCCCCGCCGGC
UAUGCCAUCCUGAAGUGUAAUGACAAGACCUUCAA
CGGCACCGGCCCUUGCCACAACGUGAGCACAGUGC
AGUGUACCCACGGCAUCAAGCCAGUGGUGAGCACA
CAGCUGCUGCUGAAUGGCUCCCUGGCCGAGGGCGA
GAUCAUCAUCCGGUCCGAGAACCUGACAAACAAUG
UGAAGACCAUCCUGGUGCACCUGAAUCAGAGCGUG
GAGAUCGUGUGCACACGGCCCAACAAUAACACCGU
GAAGUCCAUCCGCAUCGGCCCUGGCCAGACAUUCU
ACUAUACCGGCGACAUCAUCGGCGAUAUCCGGCAG
GCCCACUGUAACAUCUCCAAGUGGCACGAGACACU
GAAGCGCGUGUCUGAGAAGCUGGCCGAGCACUUCC
CUAAUAAGACAAUCAACUUUACCUCCUCUAGCGGC
GGCGACCUGGAGAUCACAACCCACUCUUUCACCUG
CCGCGGCGAGUUCUUUUACUGUAAUACAAGCGGCC
UGUUUAACUCCACAUACAUGCCCAAUGGCACCUAU
CUGCACGGCGAUACAAAUUCCAACUCCUCUAUCAC
CAUCCCUUGCAGGAUCAAGCAGAUCAUCAACAUGU
GGCAGGAAGUGGGCAGAGCCAUGUAUGCCCCUCCC
AUCGAGGGCAACAUCACCUGUAAGUCUAAUAUCAC
AGGCCUGCUGCUGGUGCGGGACGGCGGAACCGAGA
GCAAUAACACAGAGACAAAUAACACAGAGAUCUUC
CGCCCCGGCGGCGGCGACAUGAGGGAUAACUGGAG
AAGCGAGCUGUACAAGUAUAAGGUGGUGGAGAUCA
AGCCACUGGGAGUGGCACCAACCGCAUGCAAGAGG
AGAGUGGUGGGCUCUCACAGCGGCUCCGGCGGCUC
UGGCAGCGGCGGCCACGCCGCCGUGGGCAUCGGAG
CCGUGUCCCUGGGCUUUCUGGGAGUGGCAGGCUCU
ACCAUGGGAGCAGCCAGCAUGACACUGACCGUGCA
GGCAAGGCAGCUGCUGUCCGGCAUCGUGCAGCAGC
AGUCUAACCUGCUGAGAGCACCAGAGCCUCAGCAG
CACCUGCUGCAGGACACCCACUGGGGCAUCAAGCA
GCUGCAGACACGGGUGCUGGCCAUCGAGCACUACC
UGAAGGAUCAGCAGCUGCUGGGCAUCUGGGGCUGU
AGCGGCAAGCUGAUCUGCUGUACCGCCGUGCCUUG
GAAUAGCUCCUGGAGCAACAAGUCCCAGAAGGAGA
UCUGGGAUAAUAUGACAUGGAUGAACUGGUCUAAG
GAGAUCAGCAAUUACACAAACACCAUCUAUAAGCU
GCUGGAGGACUCACAGAAUCAGCAGGAAUCAAACA
ACAAAUCCCUGCUGGCACUGGAC
X1632_FJ817370_MD39_L14G8-RNA
(SEQ ID NO: 256)
AUGGACUGGACUUGGAUUCUGUUCCUGGUCGCCGC
CGCUACACGGGUGCAUUCAUCAAAUAACCUGUGGG
UCACUGUCUACUAUGGGGUGCCCGUGUGGGAGGAC
GCCGAUACCACACUGUUCUGCGCAUCCGACGCAAA
GGCAUACUCCACCGAGUCUCACAACGUGUGGGCAA
CCCACGCAUGCGUGCCAACAGACCCAAACCCCCAG
GAGAUCUAUCUGGAGAACGUGACAGAGGACUUCAA
CAUGUGGGAGAACAAUAUGGUGGAGCAGAUGCAGG
AGGACAUCAUCAGCCUGUGGGAUGAGUCCCUGAAG
CCUUGCGUGAAGCUGACCCCACUGUGCGUGACACU
GACCUGUACAAAUGUGACCAACGUGACAGACUCUG
UGGGCACAAAUAGCCGCCUGAAGGGCUACAAGGAG
GAGCUGAAGAACUGUAGCUUCAAUACCACAACCGA
GAUCAGGGAUAAGAAGAAGCAGGAGUACGCCCUGU
UUUAUAAGCUGGACAUCGUGCCAAUCAAUGAUAAC
AGCAACAAUUCCAACGGCUACAGACUGAUCAAUUG
CAACGUGUCCACCAUCAAGCAGGCCUGUCCAAAGG
UGUCUUUCGACCCUAUCCCAAUCCACUAUUGCGCA
CCAGCAGGAUUCGCAAUCCUGAAGUGUCGCGAUAA
GGAGUUUAAUGGCACCGGCACAUGCAGGAACGUGA
GCACCGUGCAGUGUACACACGGCAUCAAGCCCGUG
GUGUCUACCCAGCUGCUGCUGAAUGGCAGCCUGGC
CGAGGGCGACAUCAUCAUCAGAUCCGAGAACAUCA
CCGAUAAUGCCAAGACAAUCAUCGUGCACCUGAAC
AAGACCGUGAGCAUCACCUGCACACGCCCCAACAA
UAACACAGUGAAGUCCAUCAGGAUCGGCCCUGGCC
AGGCCCUGUACUAUACCGGAGCAAUCAUCGGCGAC
ACAAGGCAGGCCCACUGUAAUAUCAACGGCUCCGA
GUGGUACGAGAUGAUCCAGAAUGUGAAGAACAAGC
UGAAUGAGACAUUCAAGAAGAACAUCACAUUUGCC
CCCAGCUCCGGCGGCGAUCUGGAGAUCACAACCCA
CUCUUUUAACUGCCGCGGCGAGUUCUUUUAUUGUA
ACACCAGCGAGCUGUUCAAUUCUAGCCACCUGUUU
AACGGCUCUACCCUGAGCACAAACGGCACCAUCAC
ACUGCCUUGCAGGAUCAAGCAGAUCGUGCGCAUGU
GGCAGAGGGUGGGACAGGCAAUGUACGCCCCUCCC
AUCGCCGGCAAUAUCACCUGUAGAUCUAACAUCAC
CGGCCUGCUGCUGACACGGGACGGCGGAACCAACA
AGGAUACAAAUGAGGCAGAGACAUUCAGACCCGGC
GGCGGCGACAUGAGAGAUAACUGGCGGAGCGAGCU
GUACAAGUAUAAGGUGGUGAAGAUCAAGCCACUGG
GAGUGGCACCAACCAGGUGCAGGAGACGGGUGGUG
GGCAGCCACUCCGGCUCUGGCGGCAGCGGCUCCGG
CGGCCACGCAGCAAUCGGCCUGGGCACCGUGAGCC
UGGGCUUUCUGGGAACCGCAGGCUCCACAAUGGGA
GCAGCCUCUAUCACCCUGACAGUGCAGGUGAGACA
GCUGCUGAGCGGCAUCGUGCAGCAGCAGUCCAACC
UGCUGAGGGCACCAGAGCCUCAGCAGCACCUGCUG
CAGGACACCCACUGGGGCAUCAAGCAGCUGCAGGC
CCGCGUGCUGGCAGUGGAGCACUACCUGAAGGAUC
AGCAGAUCCUGGGCAUCUGGGGCUGUUCCGGCAAG
CUGAUCUGCUGUACCAACGUGCCCUGGAAUUCCUC
UUGGUCUAAUAAGUCUUAUAGCGACAUCUGGGAUA
ACCUGACAUGGAUCAAUUGGUCCAGGGAGAUCUCU
AACUACACCCAGCAGAUCUAUACACUGCUGGAAGA
AAGUCAGAAUCAGCAGGAGAAGAAUAAUCAGAGCC
UGCUGGCACUGGAU
CNE8_HM215427_MD39_L14G8-RNA
(SEQ ID NO: 257)
AUGGACUGGACUUGGAUUCUGUUCCUGGUCGCCGC
UGCUACACGAGUGCAUUCAUCUGAUAACCUGUGGG
UCACCGUCUACUAUGGCGUGCCAGUGUGGCGGGAC
GCCGAUACCACACUGUUCUGCGCCAGCGACGCCAA
GGCCUACGAUACCGAGGUGCACAACGUGUGGGCAA
CCCACGCAUGCGUGCCAACAGACCCUAAUCCACAG
GAGAUCCACCUGGAGAACGUGACAGAGAACUUCAA
CAUGUGGAAGAACAAGAUGGCCGAGCAGAUGCAGG
AGGACGUGAUCUCCCUGUGGGAUGAGUCUCUGAAG
CCCUGCGUGCAGCUGACCCCUCUGUGCGUGACACU
GAAUUGUACCAAUGCCAACCUGAAUGCCACCGUGA
AUGCCUCCACCACAAUCGGCAACAUCACAGAUGAG
GUGCGGAACUGUUCUUUCAAUACCACAACCGAGCU
GCGCGACAAGAAGCAGAACGUGUACGCCCUGUUUU
AUAAGCUGGAUAUCGUGCCCAUCAACAAUAACUCC
GAGUAUCGGCUGAUCAACUGCAAUACCUCUGUGAU
CAAGCAGGCCUGUCCUAAGGUGAGCUUCGACCCCA
UCCCUAUCCACUACUGCGCACCAGCAGGAUAUGCA
AUCCUGCGCUGUAAUGAUAAGAACUUUAAUGGCAC
AGGCCCCUGCAAGAACGUGAGCUCCGUGCAGUGUA
CCCACGGCAUCAAGCCUGUGGUGUCUACACAGCUG
CUGCUGAACGGCAGCCUGGCCGAGGACGAGAUCAU
CAUCAGGAGCGAGAACCUGACAGAUAAUGUGAAGA
CCAUCAUCGUGCACCUGAACAAGUCCGUGGAGAUC
AAUUGCACCAGGCCAUCUAAUAACACAGUGACCAG
CGUGAGAAUCGGCCCCGGCCAGGUGUUCUACUAUA
CAGGCGACAUCAUCGGCGAUAUCCGGAAGGCCUAC
UGUGAGAUCAAUCGCACAAAGUGGCACGAGACACU
GAAGCAGGUGGCCACCAAGCUGAGGGAGCACUUCA
ACAAGACAAUCAUCUUUCAGCCCCCUUCCGGCGGC
GACAUCGAGAUCACCAUGCACCACUUCAACUGCAG
AGGCGAGUUCUUUUACUGUAACACAACCAAGCUGU
UUAAUUCUACCUGGGGCGAGAACACAACCAUGGAG
GGCCACAAUGAUACAAUCGUGCUGCCUUGCAGAAU
CAAGCAGAUCGUGAACAUGUGGCAGGGAGUGGGAC
AGGCAAUGUAUGCCCCACCCAUCAGGGGCAGCAUC
AACUGCGUGAGCAAUAUCACAGGCAUCCUGCUGAC
CAGAGACGGCGGAACAAACAUGUCUAAUGAGACAU
UCAGGCCUGGCGGCGGCAACAUCAAGGAUAAUUGG
AGAAGCGAGCUGUACAAGUAUAAGGUGGUGGAGAU
CGAGCCUCUGGGCAUCGCCCCAACAAAGUGCAAGA
GGAGAGUGGUGGGCUCUCACAGCGGCUCCGGCGGC
UCUGGCAGCGGCGGCCACGCCGCCGUGGGCAUCGG
CGCCAUGAGCUUCGGCUUUCUGGGAGCAGCAGGCU
CCACCAUGGGAGCAGCCUCUAUCACACUGACCGUG
CAGGCAAGGCAGCUGCUGAGCGGCAUCGUGCAGCA
GCAGUCCAACCUGCUGAGGGCACCAGAGCCACAGC
AGCACCUGCUGCAGGACACCCACUGGGGCAUCAAG
CAGCUGCAGGCCCGCGUGCUGGCAGUGGAGCACUA
CCUGAAGGAUCAGAAGUUUCUGGGCCUGUGGGGCU
GUUCCGGCAAGAUCAUCUGCUGUACCGCCGUGCCU
UGGAACUCCACAUGGUCUAAUCGGAGCUAUGAGGA
GAUCUGGGACAACAUGACCUGGAUCAAUUGGUCCC
GCGAGAUCUCUAACUACACAAGCCAGAUCUAUGAG
AUCCUGACCGAAUCACAGAAUCAGCAGGACAGAAA
CAACAAAUCACUGCUGGAACUGGAC
CNE55_HM215418_MD39_L14G8-RNA
(SEQ ID NO: 258)
AUGGACUGGACUUGGAUUCUGUUCCUGGUCGCUGC
CGCUACACGAGUGCAUUCCUCUGAUAAACUGUGGG
UGACCGUCUACUAUGGAGUGCCAGUGUGGCGGGAC
GCCGAUACCACACUGUUCUGCGCCUCUGACGCCAA
GGCCCACGAGACAGAGGUGCACAACGUGUGGGCAA
CCCACGCAUGCGUGCCAACAGAUCCUAACCCACAG
GAGAUCCACCUGGUGAAUGUGACAGAGAACUUUAA
UAUGUGGAAGAACAAGAUGGUGGAGCAGAUGCAGG
AGGACGUGAUCAGCCUGUGGGAUGAGUCCCUGAAG
CCCUGCGUGAAGCUGACCCCUCUGUGCGUGACACU
GAACUGUACCACAGCCAACACCAAUGAGACAAAGA
ACAAUACCACAGACGAUAAUAUCAAGGACGAGAUG
AAGAACUGUACCUUCAAUAUGACCACAGAGAUCCG
GGACAAGAAGCAGCGCGUGAGCGCCCUGUUUUACA
AGCUGGAUAUCGUGCCCAUCGACGAUAGCAAGAAC
AAUUCCGAGUAUCGCCUGAUCAACUGCAAUACCAG
CGUGAUCAAGCAGGCCUGUCCUAAGGUGUCCUUCG
ACCCCAUCCCUAUCCACUACUGCACCCCAGCCGGC
UAUGUGAUCCUGAAGUGUAACGAUAAGAACUUUAA
UGGCACAGGCCCCUGCAAGAAUGUGAGCUCCGUGC
AGUGUACCCACGGCAUCAAGCCUGUGGUGUCCACA
CAGCUGCUGCUGAACGGCUCUCUGGCCGAGGAGGA
GAUCAUCAUCAGGUCUGAGAAUCUGACCGAUAACG
CCAAGAAUAUCAUCGUGCACCUGAACAAGAGCGUG
GAGAUCAAUUGCACACGGCCAUCUAACAAUACCGU
GACAAGCGUGCGCAUCGGACCAGGACAGGUGUUCU
ACUAUACCGGCGACAUCACAGGCGAUAUCAGAAAG
GCCUACUGUGAGAUCGACGGCACCGAGUGGAACAA
GACCCUGACACAGGUGGCCGAGAAGCUGAAGGAGC
ACUUUAAUAAGACCAUCGUGUACCAGCCCCCUUCC
GGCGGCGAUCUGGAGAUCACAAUGCACCACUUCAA
CUGCCGGGGCGAGUUCUUUUAUUGUAAUACCACAC
AGCUGUUUAACAAUUCUGUGGGCAACAGCACCAUC
AAGCUGCCUUGCCGCAUCAAGCAGAUCAUCAAUAU
GUGGCAGGGAGUGGGACAGGCAAUGUACGCCCCAC
CCAUCAGCGGAGCCAUCAACUGUCUGUCCAAUAUC
ACCGGCAUCCUGCUGACAAGGGACGGCGGCGGAAA
CAAUAGGUCCAAUGAGACAUUCAGGCCUGGCGGCG
GCAACAUCAAGGAUAAUUGGAGAUCUGAGCUGUAC
AAGUAUAAGGUGGUGGAGAUCGAGCCUCUGGGCAU
CGCCCCAACAAAGUGCAAGAGGAGAGUGGUGGGCU
CUCACAGCGGCUCCGGCGGCUCUGGCAGCGGCGGC
CACGCCGCCGUGGGCAUCGGCGCCAUGAGCUUCGG
CUUUCUGGGAGCAGCAGGCUCCACCAUGGGAGCAG
CCUCUAUCACCCUGACAGUGCAGGCCCGGCAGCUG
CUGUCUGGCAUCGUGCAGCAGCAGAGCAACCUGCU
GAGGGCACCAGAGCCACAGCAGCACAUGCUGCAGG
ACACACACUGGGGCAUCAAGCAGCUGCAGGCCAGG
GUGCUGGCAGUGGAGCACUACCUGAAGGAUCAGAG
AUUUCUGGGCCUGUGGGGCUGUAGCGGCAAGACCA
UCUGCUGUACAGCCGUGCCUUGGAACUCCACCUGG
UCUAAUAAGACAUAUGAGGAGAUCUGGGACAACAU
GACCUGGACAAAUUGGUCCCGGGAGAUCUCUAACU
ACACCAAUCAGAUCUAUUCCAUUCUGACCGAAUCA
CAGUCACAGCAGGAUAAAAAUAACAAAAGUCUGCU
GGAACUGGAU
AD8_MD64_link14_TS1-RNA
(SEQ ID NO: 259)
GGAUCCGCCACCAUGGACUGGACUUGGAUUCUGUU
CCUGGUCGCCGCCGCUACUCGGGUGCAUUCUGUCG
AAAACCUGUGGGUGACUGUCUAUUAUGGAGUGCCC
GUGUGGAAGGAGGCCACCACAACCCUGUUCUGCGC
CUCCGACGCCAAGGCCUACGAUACCGAGGUGCACA
ACGUGUGGGCCACCCACGAGUGCGUGCCUACAGAC
CCAAACCCCCAGGAGGUGGUGCUGGAGAAUGUGAC
AGAGAACUUCAACAUGUGGAAGAACAAUAUGGUGG
AGCAGAUGCACGAGGACAUCAUCGAGCUGUGGGAU
CAGAGCCUGAAGCCUUGCGUGAAGCUGACCCCACU
GUGCGUGACCCUGAAUUGUACAGACCUGCGGAAUG
UGACAAACAUCAACAAUAGCUCCGAGGGCAUGAGA
GGCGAGAUCAAGAAUUGUAGCUUCAACAUCACAAC
CUCCAUCAGGGACAAGGUGAAGAAGGAUUACGCCC
UGUUUUAUCGCCUGGAUGUGGUGCCCAUCGACAAU
GAUAACACCUCUUACCGGCUGAUCAAUUGCAACAC
AAGCACCAUCACACAGGCCUGUCCAAAGGUGUCCU
UCGAGCCUAUCCCAAUCCACUAUUGCACCCCCGCC
GGCUUCGCCAUCCUGAAGUGUAAGGACAAGAAGUU
UAACGGCACAGGCCCUUGCAAGAACGUGAGCACCG
UGCAGUGUACACACGGCAUCCGGCCAGUGGUGAGC
ACCCAGCUGCUGCUGAACGGCUCCCUGGCAGAGGA
GGAAGUGAUCAUCAGAUCUAGCAAUUUCACAGAUA
AUGCCAAGAACAUCAUCGUGCAGCUGAAGGAGUCC
GUGGAGAUCAACUGCACCCGGCCCAACAAUAACAC
AGUGAAGUCUAUCCACAUCGGCCCUGGCAGAGCCU
UUUACUAUACCGGCGACAUCAUCGGCGAUAUCAGG
CAGGCCCACUGUAACAUCAGCCGCACCAAGUGGAA
UAACACACUGAAUCAGAUCGCCACCAAGCUGAAGG
AGCAGUUCGGCAAUAACAAGACAAUCGUGUUUAAC
CAGUCCUCUGGCGGCGACCCAGAGAUCGUGAUGCA
CUCUUUUAAUUGCGGCGGCGAGUUCUUUUACUGUA
ACUCUACCCAGCUGUUCAAUAGCACAUGGAACUUC
AACGGCACCUGGAAUCUGACACAGAGCAACGGCAC
CGAGGGCAAUGAUACCAUCACACUGCCCUGCAGGA
UCAAGCAGAUCAUCAACAUGUGGCAGGAAGUGGGC
AAGGCCAUGUAUGCCCCUCCCAUCAGGGGCCAGAU
CCGCUGUAGCUCCAAUAUCACCGGCCUGAUCCUGA
CAAGGGACGGCGGAAAUAACCACAAUAACGAUACC
GAGACAUUCCGCCCCGGCGGCGGCGACAUGAGGGA
UAACUGGAGAUCCGAGCUGUACAAGUAUAAGGUGG
UGAAGAUCGAGCCACUGGGAGUGGCACCAACCAAG
UGCAAGAGGAGAGUGGUGCAGUCUCACAGCGGCUC
CGGCGGCUCUGGCAGCGGCGGCCACGCCGCCGUGG
GCACCAUCGGCGCCAUGAGCCUGGGCUUUCUGGGA
GCAGCAGGCUCCACAAUGGGAGCAGCCUCUAUCAC
CCUGACAGUGCAGGCCAGGCUGCUGCUGUCCGGCA
UCGUGCAGCAGCAGAAUAACCUGCUGAGGGCACCA
GAGCCUCAGCAGCACCUGCUGCAGCUGACCGUGUG
GGGCAUCAAGCAGCUGCAGGCCCGGGUGCUGGCAG
UGGAGCACUAUCUGAGAGAUCAGCAGCUGCUGGGA
AUCUGGGGAUGCAGCGGCAAGCUGAUCUGCUGUAC
CGCCGUGCCAUGGAACGCCUCCUGGUCUAAUAAGA
CCCUGGACAUGAUCUGGAAUAACAUGACAUGGAUG
GAGUGGGAGCGCGAGAUCGAUAACUACACCGGCCU
GAUCUAUACACUGAUCGAGGAAUCACAGAAUCAGC
AGGAGAAAAACGAACAGGAACUGCUGGAACUGGAU
GGCGGCGUCGAAAAUCUCUGGGUCACCGUCUAUUA
UGGGGUCCCUGUCUGGAAGGAAGCAACUACUACUC
UGUUCUGUGCCUCCGAUGCCAAGGCCUACGACACA
GAGGUGCACAACGUGUGGGCUACACACGAGUGCGU
GCCAACCGAUCCAAACCCCCAGGAGGUGGUGCUGG
AGAACGUGACCGAGAACUUCAACAUGUGGAAGAAC
AACAUGGUGGAGCAGAUGCACGAGGACAUCAUCGA
GCUGUGGGAUCAGUCCCUGAAGCCUUGCGUGAAGC
UGACACCACUGUGCGUGACACUGAACUGUACCGAC
CUGAGGAACGUGACCAACAUCAACAACAGCUCCGA
GGGAAUGAGAGGCGAGAUCAAGAACUGUAGCUUCA
ACAUCACCACAUCCAUCCGGGACAAGGUGAAGAAG
GAUUACGCCCUGUUUUACCGCCUGGAUGUGGUGCC
CAUCGACAACGAUAACACCUCUUACAGGCUGAUCA
ACUGCAACACCAGCACAAUCACCCAGGCUUGUCCA
AAGGUGUCCUUUGAGCCUAUCCCAAUCCACUACUG
CACACCCGCCGGCUUCGCUAUCCUGAAGUGUAAGG
ACAAGAAGUUUAACGGAACCGGCCCUUGCAAGAAC
GUGUCUACAGUGCAGUGUACCCACGGCAUCAGGCC
AGUGGUGAGCACACAGCUGCUGCUGAACGGCAGCC
UGGCCGAGGAGGAAGUGAUCAUCAGAUCUAGCAAC
UUCACCGAUAACGCUAAGAACAUCAUCGUGCAGCU
GAAGGAGUCCGUGGAGAUCAACUGCACAAGGCCCA
ACAACAACACCGUGAAGUCUAUCCACAUCGGACCU
GGCAGAGCCUUUUACUACACAGGAGACAUCAUCGG
CGAUAUCCGGCAGGCUCACUGUAACAUCAGCCGCA
CAAAGUGGAACAACACCCUGAACCAGAUCGCCACA
AAGCUGAAGGAGCAGUUCGGCAACAACAAGACCAU
CGUGUUUAACCAGUCCAGCGGCGGCGACCCCGAGA
UCGUGAUGCACUCUUUCAACUGCGGCGGAGAGUUC
UUUUACUGUAACUCUACACAGCUGUUCAACAGCAC
CUGGAACUUUAACGGAACAUGGAACCUGACCCAGA
GCAACGGAACCGAGGGCAACGAUACAAUCACCCUG
CCUUGCCGGAUCAAGCAGAUCAUCAACAUGUGGCA
GGAAGUGGGAAAGGCCAUGUACGCUCCCCCUAUCA
GGGGACAGAUCAGGUGUAGCUCCAACAUCACAGGA
CUGAUCCUGACCCGGGACGGCGGAAACAACCACAA
CAACGAUACAGAGACAUUCAGGCCUGGCGGAGGCG
ACAUGAGGGAUAACUGGAGAUCCGAGCUGUACAAG
UACAAGGUGGUGAAGAUCGAGCCACUGGGAGUGGC
UCCAACCAAGUGCAAGAGGAGAGUGGUGCAGUCUC
ACAGCGGCAGCGGCGGCAGCGGCAGCGGAGGCCAC
GCUGCUGUGGGAACAAUCGGAGCUAUGAGCCUGGG
AUUUCUGGGAGCUGCUGGCAGCACCAUGGGAGCUG
CUUCUAUCACACUGACCGUGCAGGCUAGGCUGCUG
CUGUCCGGAAUCGUGCAGCAGCAGAACAACCUGCU
GAGGGCUCCAGAGCCUCAGCAGCACCUGCUGCAGC
UGACAGUGUGGGGCAUCAAGCAGCUGCAGGCCAGG
GUGCUGGCUGUGGAGCACUACCUGAGGGACCAGCA
GCUGCUGGGCAUCUGGGGAUGUAGCGGCAAGCUGA
UCUGCUGUACCGCCGUGCCAUGGAACGCUUCCUGG
UCUAACAAGACACUGGACAUGAUCUGGAACAACAU
GACCUGGAUGGAGUGGGAGCGCGAGAUCGAUAACU
ACACAGGCCUGAUCUACACCCUGAUCGAAGAAAGU
CAGAAUCAGCAGGAAAAGAACGAACAGGAACUGCU
GGAACUGGACGGUGGCGUCGAGAAUCUGUGGGUCA
CCGUCUAUUAUGGAGUCCCCGUCUGGAAAGAGGCU
ACUACUACACUGUUUUGUGCAAGCGAUGCCAAGGC
CUACGACACAGAGGUGCACAACGUGUGGGCCACAC
ACGAGUGCGUGCCAACCGAUCCAAACCCCCAGGAG
GUGGUGCUGGAGAAUGUGACCGAGAAUUUCAACAU
GUGGAAGAACAAUAUGGUGGAGCAGAUGCACGAGG
ACAUCAUCGAGCUGUGGGAUCAGUCCCUGAAGCCU
UGCGUGAAGCUGACACCACUGUGCGUGACACUGAA
CUGUACCGACCUGAGGAAUGUGACCAACAUCAACA
AUAGCUCCGAGGGCAUGAGAGGCGAGAUCAAGAAU
UGUAGCUUCAACAUCACCACAUCCAUCCGGGACAA
GGUGAAGAAGGAUUACGCCCUGUUUUAUCGCCUGG
AUGUGGUGCCCAUCGACAAUGAUAACACCUCUUAC
AGGCUGAUCAAUUGCAACACCAGCACAAUCACCCA
GGCCUGUCCAAAGGUGUCCUUUGAGCCUAUCCCAA
UCCACUAUUGCACACCCGCCGGCUUCGCCAUCCUG
AAGUGUAAGGACAAGAAGUUUAACGGCACCGGCCC
UUGCAAGAACGUGAGCACAGUGCAGUGUACCCACG
GCAUCAGGCCAGUGGUGAGCACACAGCUGCUGCUG
AACGGCUCCCUGGCCGAGGAGGAAGUGAUCAUCAG
AUCUAGCAAUUUCACCGAUAAUGCCAAGAACAUCA
UCGUGCAGCUGAAGGAGUCCGUGGAGAUCAACUGC
ACAAGGCCCAACAAUAACACCGUGAAGUCUAUCCA
CAUCGGCCCUGGCAGAGCCUUUUACUAUACCGGCG
ACAUCAUCGGCGAUAUCCGGCAGGCCCACUGUAAC
AUCAGCCGCACAAAGUGGAAUAACACCCUGAAUCA
GAUCGCCACAAAGCUGAAGGAGCAGUUCGGCAAUA
ACAAGACCAUCGUGUUUAACCAGUCCUCUGGCGGC
GACCCCGAGAUCGUGAUGCACUCUUUCAAUUGCGG
CGGCGAGUUCUUUUACUGUAACUCUACACAGCUGU
UCAAUAGCACCUGGAACUUCAACGGCACAUGGAAU
CUGACCCAGAGCAACGGCACCGAGGGCAAUGAUAC
AAUCACCCUGCCUUGCCGGAUCAAGCAGAUCAUCA
ACAUGUGGCAGGAAGUGGGCAAGGCCAUGUAUGCC
CCUCCCAUCAGGGGACAGAUCAGGUGUAGCUCCAA
UAUCACAGGCCUGAUCCUGACCCGGGACGGCGGAA
AUAACCACAAUAACGAUACAGAGACAUUCAGGCCC
GGCGGCGGCGACAUGAGGGAUAACUGGAGAUCCGA
GCUGUACAAGUAUAAGGUGGUGAAGAUCGAGCCAC
UGGGAGUGGCACCAACCAAGUGCAAGAGGAGAGUG
GUGCAGUCUCACAGCGGCUCCGGCGGCUCUGGCAG
CGGCGGCCACGCAGCAGUGGGAACAAUCGGAGCAA
UGAGCCUGGGCUUUCUGGGAGCAGCAGGCUCCACC
AUGGGAGCAGCCUCUAUCACACUGACCGUGCAGGC
AAGGCUGCUGCUGUCCGGCAUCGUGCAGCAGCAGA
AUAACCUGCUGAGGGCACCAGAGCCUCAGCAGCAC
CUGCUGCAGCUGACAGUGUGGGGCAUCAAGCAGCU
GCAGGCCAGGGUGCUGGCAGUGGAGCACUAUCUGA
GGGACCAGCAGCUGCUGGGCAUCUGGGGCUGUAGC
GGCAAGCUGAUCUGCUGUACCGCCGUGCCCUGGAA
CGCCUCCUGGUCUAAUAAGACACUGGACAUGAUCU
GGAAUAACAUGACCUGGAUGGAGUGGGAGCGCGAG
AUCGAUAACUACACAGGCCUGAUCUAUACCCUGAU
UGAGGAGUCACAGAACCAGCAGGAAAAGAACGAAC
AGGAACUGCUGGAACUGGAUUGAUAACUCGAG
AD8_MD64_link14-RNA
(SEQ ID NO: 260)
GUGUGGGGCAUCAAGCAGCUGCAGGCCCGGGUGCU
GGCAGUGGAGCACUAUCUGAGAGAUCAGCAGCUGC
UGGGAAUCUGGGGAUGCAGCGGCAAGCUGAUCUGC
UGUACCGCCGUGCCAUGGAACGCCUCCUGGUCUAA
UAAGACCCUGGACAUGAUCUGGAAUAACAUGACAU
GGAUGGAGUGGGAGCGCGAGAUCGAUAACUACACC
GGCCUGAUCUAUACACUGAUCGAGGAAUCACAGAA
UCAGCAGGAGAAAAACGAACAGGAACUGCUGGAAC
UGGAUUGAUAACUCGAGCAUUCUGUCGAAAACCUG
UGGGUGACUGUCUAUUAUGGAGUGCCCGUGUGGAA
GGAGGCCACCACAACCCUGUUCUGCGCCUCCGACG
CCAAGGCCUACGAUACCGAGGUGCACAACGUGUGG
GCCACCCACGAGUGCGUGCCUACAGACCCAAACCC
CCAGGAGGUGGUGCUGGAGAAUGUGACAGAGAACU
UCAACAUGUGGAAGAACAAUAUGGUGGAGCAGAUG
CACGAGGACAUCAUCGAGCUGUGGGAUCAGAGCCU
GAAGCCUUGCGUGAAGCUGACCCCACUGUGCGUGA
CCCUGAAUUGUACAGACCUGCGGAAUGUGACAAAC
AUCAACAAUAGCUCCGAGGGCAUGAGAGGCGAGAU
CAAGAAUUGUAGCUUCAACAUCACAACCUCCAUCA
GGGACAAGGUGAAGAAGGAUUACGCCCUGUUUUAU
CGCCUGGAUGUGGUGCCCAUCGACAAUGAUAACAC
CUCUUACCGGCUGAUCAAUUGCAACACAAGCACCA
UCACACAGGCCUGUCCAAAGGUGUCCUUCGAGCCU
AUCCCAAUCCACUAUUGCACCCCCGCCGGCUUCGC
CAUCCUGAAGUGUAAGGACAAGAAGUUUAACGGCA
CAGGCCCUUGCAAGAACGUGAGCACCGUGCAGUGU
ACACACGGCAUCCGGCCAGUGGUGAGCACCCAGCU
GCUGCUGAACGGCUCCCUGGCAGAGGAGGAAGUGA
UCAUCAGAUCUAGCAAUUUCACAGAUAAUGCCAAG
AACAUCAUCGUGCAGCUGAAGGAGUCCGUGGAGAU
CAACUGCACCCGGCCCAACAAUAACACAGUGAAGU
CUAUCCACAUCGGCCCUGGCAGAGCCUUUUACUAU
ACCGGCGACAUCAUCGGCGAUAUCAGGCAGGCCCA
CUGUAACAUCAGCCGCACCAAGUGGAAUAACACAC
UGAAUCAGAUCGCCACCAAGCUGAAGGAGCAGUUC
GGCAAUAACAAGACAAUCGUGUUUAACCAGUCCUC
UGGCGGCGACCCAGAGAUCGUGAUGCACUCUUUUA
AUUGCGGCGGCGAGUUCUUUUACUGUAACUCUACC
CAGCUGUUCAAUAGCACAUGGAACUUCAACGGCAC
CUGGAAUCUGACACAGAGCAACGGCACCGAGGGCA
AUGAUACCAUCACACUGCCCUGCAGGAUCAAGCAG
AUCAUCAACAUGUGGCAGGAAGUGGGCAAGGCCAU
GUAUGCCCCUCCCAUCAGGGGCCAGAUCCGCUGUA
GCUCCAAUAUCACCGGCCUGAUCCUGACAAGGGAC
GGCGGAAAUAACCACAAUAACGAUACCGAGACAUU
CCGCCCCGGCGGCGGCGACAUGAGGGAUAACUGGA
GAUCCGAGCUGUACAAGUAUAAGGUGGUGAAGAUC
GAGCCACUGGGAGUGGCACCAACCAAGUGCAAGAG
GAGAGUGGUGCAGUCUCACAGCGGCUCCGGCGGCU
CUGGCAGCGGCGGCCACGCCGCCGUGGGCACCAUC
GGCGCCAUGAGCCUGGGCUUUCUGGGAGCAGCAGG
CUCCACAAUGGGAGCAGCCUCUAUCACCCUGACAG
UGCAGGCCAGGCUGCUGCUGUCCGGCAUCGUGCAG
CAGCAGAAUAACCUGCUGAGGGCACCAGAGCCUCA
GCAGCACCUGCUGCAGCUGACC
001428_MD39_link14_TS1-RNA
(SEQ ID NO: 261)
GGAUCCGCCACCAUGGACUGGACUUGGAUUCUGUU
CCUGGUGGCAGCAGCAACUAGAGUGCAUUCCGUCG
AAAACCUGUGGGUGACCGUGUAUUAUGGAGUGCCC
GUGUGGAAGGAGGCCCGGACCACACUGUUCUGCGC
CUCCGACGCCAAGGCCUACGAGACAGAGGUGCACA
ACGUGUGGGCCACACACGCCUGCGUGCCUACCGAU
CCAAAUCCCCAGGAGAUGGUGCUGGGCAACGUGAC
CGAGAACUUUAAUAUGUGGAAGAACGACAUGGUGG
AUCAGAUGCACGAGGACGUGAUCUCUCUGUGGGCC
CAGAGCCUGAAGCCUUGCGUGAAGCUGACCCCACU
GUGCGUGACACUGGAGUGUACCCAGGUGAACGCCA
CACAGGGCAAUACCACACAGGUGAACGUGACCCAA
GUGAAUGGCGACGAGAUGAAGAACUGUUCCUUCAA
UACCACAACCGAGAUCCGGGAUAAGAAGCAGAAGG
CCUACGCCCUGUUUUAUAGACUGGACCUGGUGCCU
CUGGAGCGGGAGAACAGAGGCGAUUCUAAUAGCGC
CUCCAAGUAUAUCCUGAUCAACUGCAAUACAUCUG
CCAUCACCCAGGCCUGUCCUAAAGUGAAUUUCGAU
CCUAUCCCAAUCCACUACUGCACCCCAGCCGGCUA
UGCCAUCCUGAAGUGUAACAACAAGACCUUCAACG
GCACCGGCUCCUGCAACAACGUGAGCACAGUGCAG
UGUACCCACGGCAUCAAGCCAGUGGUGAGCACCCA
GCUGCUGCUGAACGGCUCCCUGGCAGAGGAGGAGA
UCAUCAUCAGGUCCGAGAACCUGACAGACAAUGUG
AAGACCAUCAUCGUGCACCUGGAUCAGUCCGUGGA
GAUCGUGUGCACACGGCCAAACAAUAACACCGUGA
AGUCUAUCAGAAUCGGCCCCGGCCAGACAUUCUAC
UAUACCGGCGACAUCAUCGGCAAUAUCCGGGAGGC
CCACUGUAACAUCUCUGAGAAGAAGUGGCACGAGA
UGCUGCGGAGAGUGAGCGAGAAGCUGGCCGAGCAC
UUCCCCAAUAAGACAAUCAAGUUUACCAGCUCCUC
UGGCGGCGAUCUGGAGAUCACAACCCACAGCUUCA
ACUGCAGAGGCGAGUUCUUUUACUGUAACACCAGC
GGCCUGUUUAAUUCCACAUACAUGCCCAACGGCAC
CUAUAUGCCUAAUGGCACAAAUAACUCUAACAGCA
CCAUCAUCCUGCCAUGCCGGAUCAAGCAGAUCAUC
AAUAUGUGGCAGGAAGUGGGCAGAGCCAUGUAUGC
CCCUCCCAUCGCCGGCAACAUCACAUGUAACAGCA
AUAUCACCGGCCUGCUGCUGGUGAGGGACGGCGGC
AAGAAUAACAAUACAGAGAUCUUCCGCCCCGGCGG
CGGCGACAUGAGGGAUAACUGGCGCUCCGAGCUGU
ACAAGUAUAAGGUGGUGGAGAUCAAGCCACUGGGA
GUGGCACCAACCAGGUGCAAGAGGCGCGUGGUGGG
CUCCCACUCUGGCAGCGGCGGCUCCGGCUCUGGCG
GCCACGCAGCAGUGGGCCUGGGAGCCGUGAGCCUG
GGCUUUCUGGGAGCAGCAGGCUCUACCAUGGGAGC
AGCCAGCAUCACACUGACCGUGCAGGCAAGGCAGC
UGCUGUCCGGCAUCGUGCAGCAGCAGUCUAACCUG
CUGCAGGCACCAGAGCCUCAGCAGCACCUGCUGCA
GGACACACACUGGGGCAUCAAGCAGCUGCAGACCC
GCGUGCUGGCCAUCGAGCACUACCUGAAGGAUCAG
CAGCUGCUGGGCAUCUGGGGCUGCUCUGGCAAGCU
GAUCUGCUGUACAGCCGUGCCUUGGAACAGCUCCU
GGAGCAAUAAGUCCCUGACAGACAUCUGGGAUAAU
AUGACCUGGAUGCAGUGGGAUAGGGAGGUGAGCAA
CUACACCGGCAUCAUCUAUCGCCUGCUGGAAGACU
CACAGAAUCAGCAGGAAAGGAAUGAACAGGAUCUG
CUGGCACUGGACGGGGGAGUCGAGAACCUCUGGGU
CACCGUGUAUUAUGGAGUCCCCGUCUGGAAAGAAG
CCCGAACCACCCUGUUUUGUGCCUCUGAUGCUAAA
GCCUACGAGACAGAGGUGCACAACGUGUGGGCUAC
ACACGCUUGCGUGCCAACCGACCCAAACCCCCAGG
AGAUGGUGCUGGGCAACGUGACCGAGAACUUCAAC
AUGUGGAAGAACGACAUGGUGGAUCAGAUGCACGA
GGAUGUGAUCUCUCUGUGGGCCCAGAGCCUGAAGC
CUUGCGUGAAGCUGACCCCACUGUGCGUGACACUG
GAGUGUACCCAGGUGAACGCUACACAGGGCAACAC
CACACAGGUGAACGUGACCCAGGUGAACGGAGACG
AGAUGAAGAACUGUUCCUUCAACACCACAACCGAG
AUCAGGGAUAAGAAGCAGAAGGCCUACGCUCUGUU
UUACAGACUGGACCUGGUGCCACUGGAGAGGGAGA
ACAGAGGCGAUUCUAACAGCGCCUCCAAGUACAUC
CUGAUCAACUGCAACACAUCUGCCAUCACCCAGGC
UUGUCCUAAGGUGAACUUCGACCCUAUCCCAAUCC
ACUACUGCACACCAGCCGGCUACGCUAUCCUGAAG
UGUAACAACAAGACCUUCAACGGAACCGGCUCCUG
CAACAACGUGUCUACAGUGCAGUGUACCCACGGCA
UCAAGCCCGUGGUGAGCACCCAGCUGCUGCUGAAC
GGCAGCCUGGCUGAGGAGGAGAUCAUCAUCCGGUC
CGAGAACCUGACAGACAACGUGAAGACCAUCAUCG
UGCACCUGGAUCAGUCCGUGGAGAUCGUGUGCACA
AGGCCAAACAACAACACCGUGAAGUCUAUCAGAAU
CGGACCCGGCCAGACCUUCUACUACACCGGAGACA
UCAUCGGCAACAUCAGGGAGGCCCACUGUAACAUC
UCUGAGAAGAAGUGGCACGAGAUGCUGAGGAGAGU
GAGCGAGAAGCUGGCUGAGCACUUCCCUAACAAGA
CAAUCAAGUUUACCAGCUCCUCUGGCGGAGAUCUG
GAGAUCACAACCCACAGCUUCAACUGCAGAGGAGA
GUUCUUUUACUGUAACACCAGCGGCCUGUUUAACU
CCACAUACAUGCCCAACGGAACCUACAUGCCUAAC
GGCACAAACAACUCUAACAGCACCAUCAUCCUGCC
CUGCAGGAUCAAGCAGAUCAUCAACAUGUGGCAGG
AAGUGGGAAGAGCCAUGUACGCUCCCCCUAUCGCC
GGCAACAUCACAUGUAACAGCAACAUCACCGGACU
GCUGCUGGUGCGGGACGGCGGAAAGAACAACAACA
CAGAGAUCUUCCGCCCUGGCGGAGGCGACAUGAGG
GAUAACUGGCGCUCCGAGCUGUACAAGUACAAGGU
GGUGGAGAUCAAGCCACUGGGAGUGGCUCCAACCA
GGUGCAAGAGGAGGGUGGUGGGCAGCCACUCUGGC
AGCGGAGGCUCCGGAUCUGGAGGCCACGCUGCUGU
GGGACUGGGAGCCGUGAGCCUGGGAUUUCUGGGAG
CUGCUGGAUCUACCAUGGGAGCUGCUAGCAUCACA
CUGACCGUGCAGGCUAGGCAGCUGCUGUCCGGAAU
CGUGCAGCAGCAGUCUAACCUGCUGCAGGCUCCCG
AGCCUCAGCAGCACCUGCUGCAGGACACACACUGG
GGCAUCAAGCAGCUGCAGACCCGCGUGCUGGCCAU
CGAGCACUACCUGAAGGAUCAGCAGCUGCUGGGCA
UCUGGGGAUGUUCUGGCAAGCUGAUCUGCUGUACA
GCUGUGCCAUGGAACAGCUCCUGGAGCAACAAGUC
CCUGACAGACAUCUGGGAUAACAUGACCUGGAUGC
AGUGGGAUCGGGAGGUGAGCAACUACACCGGCAUC
AUCUACCGCCUGCUGGAAGACUCACAGAAUCAGCA
GGAACGGAAUGAACAGGACCUCCUCGCACUGGAUG
GCGGAGUCGAAAACCUGUGGGUCACCGUCUACUAU
GGAGUGCCAGUGUGGAAAGAGGCUAGGACUACCCU
GUUCUGUGCCAGCGAUGCCAAAGCCUACGAGACAG
AGGUGCACAACGUGUGGGCAACACACGCAUGCGUG
CCAACCGACCCAAAUCCCCAGGAGAUGGUGCUGGG
CAACGUGACCGAGAACUUCAAUAUGUGGAAGAACG
ACAUGGUGGAUCAGAUGCACGAGGAUGUGAUCUCU
CUGUGGGCCCAGAGCCUGAAGCCUUGCGUGAAGCU
GACCCCACUGUGCGUGACACUGGAGUGUACCCAGG
UGAACGCCACACAGGGCAAUACCACACAGGUGAAC
GUGACCCAAGUGAAUGGCGACGAGAUGAAGAACUG
UUCCUUCAAUACCACAACCGAGAUCAGGGAUAAGA
AGCAGAAGGCCUACGCCCUGUUUUAUAGACUGGAC
CUGGUGCCACUGGAGAGGGAGAACAGAGGCGAUUC
UAAUAGCGCCUCCAAGUAUAUCCUGAUCAACUGCA
AUACAUCUGCCAUCACCCAGGCCUGUCCUAAAGUG
AAUUUCGACCCUAUCCCAAUCCACUACUGCACACC
AGCCGGCUAUGCCAUCCUGAAGUGUAACAACAAGA
CCUUCAACGGCACCGGCUCCUGCAACAACGUGAGC
ACAGUGCAGUGUACCCACGGCAUCAAGCCCGUGGU
GAGCACCCAGCUGCUGCUGAACGGCUCCCUGGCAG
AGGAGGAGAUCAUCAUCCGGUCCGAGAACCUGACA
GACAAUGUGAAGACCAUCAUCGUGCACCUGGAUCA
GUCCGUGGAGAUCGUGUGCACAAGGCCAAACAAUA
ACACCGUGAAGUCUAUCAGAAUCGGCCCCGGCCAG
ACCUUCUACUAUACCGGCGACAUCAUCGGCAAUAU
CAGGGAGGCCCACUGUAACAUCUCUGAGAAGAAGU
GGCACGAGAUGCUGAGGAGAGUGAGCGAGAAGCUG
GCCGAGCACUUCCCUAAUAAGACAAUCAAGUUUAC
CAGCUCCUCUGGCGGCGAUCUGGAGAUCACAACCC
ACAGCUUCAACUGCAGAGGCGAGUUCUUUUACUGU
AACACCAGCGGCCUGUUUAAUUCCACAUACAUGCC
CAACGGCACCUAUAUGCCUAAUGGCACAAAUAACU
CUAACAGCACCAUCAUCCUGCCCUGCAGGAUCAAG
CAGAUCAUCAAUAUGUGGCAGGAAGUGGGCAGAGC
CAUGUAUGCCCCUCCCAUCGCCGGCAACAUCACAU
GUAACAGCAAUAUCACCGGCCUGCUGCUGGUGCGG
GACGGCGGCAAGAAUAACAAUACAGAGAUCUUCCG
CCCCGGCGGCGGCGACAUGAGGGAUAACUGGCGCU
CCGAGCUGUACAAGUAUAAGGUGGUGGAGAUCAAG
CCACUGGGAGUGGCACCAACCAGGUGCAAGAGGCG
CGUGGUGGGCUCCCACUCUGGCAGCGGCGGCUCCG
GCUCUGGCGGCCACGCAGCAGUGGGCCUGGGAGCC
GUGUCCCUGGGCUUUCUGGGAGCAGCAGGCUCUAC
CAUGGGAGCAGCCAGCAUCACACUGACCGUGCAGG
CAAGGCAGCUGCUGUCCGGCAUCGUGCAGCAGCAG
UCUAACCUGCUGCAGGCACCAGAGCCUCAGCAGCA
CCUGCUGCAGGACACACACUGGGGCAUCAAGCAGC
UGCAGACCCGCGUGCUGGCCAUCGAGCACUACCUG
AAGGAUCAGCAGCUGCUGGGCAUCUGGGGCUGUUC
UGGCAAGCUGAUCUGCUGUACAGCCGUGCCAUGGA
ACAGCUCCUGGAGCAAUAAGUCCCUGACAGACAUC
UGGGAUAAUAUGACCUGGAUGCAGUGGGAUCGGGA
GGUGAGCAACUACACCGGCAUCAUCUAUCGCCUGC
UGGAGGACUCACAGAAUCAGCAGGAGCGGAACGAA
CAGGAUCUGCUGGCACUGGAUUGAUAACUCGAG
001428_MD39_link14-RNA
(SEQ ID NO: 262)
GGAUCCGCCACCAUGGACUGGACUUGGAUUCUGUU
CCUGGUGGCAGCAGCAACUAGAGUGCAUUCCGUCG
AAAACCUGUGGGUGACCGUGUAUUAUGGAGUGCCC
GUGUGGAAGGAGGCCCGGACCACACUGUUCUGCGC
CUCCGACGCCAAGGCCUACGAGACAGAGGUGCACA
ACGUGUGGGCCACACACGCCUGCGUGCCUACCGAU
CCAAAUCCCCAGGAGAUGGUGCUGGGCAACGUGAC
CGAGAACUUUAAUAUGUGGAAGAACGACAUGGUGG
AUCAGAUGCACGAGGACGUGAUCUCUCUGUGGGCC
CAGAGCCUGAAGCCUUGCGUGAAGCUGACCCCACU
GUGCGUGACACUGGAGUGUACCCAGGUGAACGCCA
CACAGGGCAAUACCACACAGGUGAACGUGACCCAA
GUGAAUGGCGACGAGAUGAAGAACUGUUCCUUCAA
UACCACAACCGAGAUCCGGGAUAAGAAGCAGAAGG
CCUACGCCCUGUUUUAUAGACUGGACCUGGUGCCU
CUGGAGCGGGAGAACAGAGGCGAUUCUAAUAGCGC
CUCCAAGUAUAUCCUGAUCAACUGCAAUACAUCUG
CCAUCACCCAGGCCUGUCCUAAAGUGAAUUUCGAU
CCUAUCCCAAUCCACUACUGCACCCCAGCCGGCUA
UGCCAUCCUGAAGUGUAACAACAAGACCUUCAACG
GCACCGGCUCCUGCAACAACGUGAGCACAGUGCAG
UGUACCCACGGCAUCAAGCCAGUGGUGAGCACCCA
GCUGCUGCUGAACGGCUCCCUGGCAGAGGAGGAGA
UCAUCAUCAGGUCCGAGAACCUGACAGACAAUGUG
AAGACCAUCAUCGUGCACCUGGAUCAGUCCGUGGA
GAUCGUGUGCACACGGCCAAACAAUAACACCGUGA
AGUCUAUCAGAAUCGGCCCCGGCCAGACAUUCUAC
UAUACCGGCGACAUCAUCGGCAAUAUCCGGGAGGC
CCACUGUAACAUCUCUGAGAAGAAGUGGCACGAGA
UGCUGCGGAGAGUGAGCGAGAAGCUGGCCGAGCAC
UUCCCCAAUAAGACAAUCAAGUUUACCAGCUCCUC
UGGCGGCGAUCUGGAGAUCACAACCCACAGCUUCA
ACUGCAGAGGCGAGUUCUUUUACUGUAACACCAGC
GGCCUGUUUAAUUCCACAUACAUGCCCAACGGCAC
CUAUAUGCCUAAUGGCACAAAUAACUCUAACAGCA
CCAUCAUCCUGCCAUGCCGGAUCAAGCAGAUCAUC
AAUAUGUGGCAGGAAGUGGGCAGAGCCAUGUAUGC
CCCUCCCAUCGCCGGCAACAUCACAUGUAACAGCA
AUAUCACCGGCCUGCUGCUGGUGAGGGACGGCGGC
AAGAAUAACAAUACAGAGAUCUUCCGCCCCGGCGG
CGGCGACAUGAGGGAUAACUGGCGCUCCGAGCUGU
ACAAGUAUAAGGUGGUGGAGAUCAAGCCACUGGGA
GUGGCACCAACCAGGUGCAAGAGGCGCGUGGUGGG
CUCCCACUCUGGCAGCGGCGGCUCCGGCUCUGGCG
GCCACGCAGCAGUGGGCCUGGGAGCCGUGAGCCUG
GGCUUUCUGGGAGCAGCAGGCUCUACCAUGGGAGC
AGCCAGCAUCACACUGACCGUGCAGGCAAGGCAGC
UGCUGUCCGGCAUCGUGCAGCAGCAGUCUAACCUG
CUGCAGGCACCAGAGCCUCAGCAGCACCUGCUGCA
GGACACACACUGGGGCAUCAAGCAGCUGCAGACCC
GCGUGCUGGCCAUCGAGCACUACCUGAAGGAUCAG
CAGCUGCUGGGCAUCUGGGGCUGCUCUGGCAAGCU
GAUCUGCUGUACAGCCGUGCCUUGGAACAGCUCCU
GGAGCAAUAAGUCCCUGACAGACAUCUGGGAUAAU
AUGACCUGGAUGCAGUGGGAUAGGGAGGUGAGCAA
CUACACCGGCAUCAUCUAUCGCCUGCUGGAAGACU
CACAGAAUCAGCAGGAAAGGAAUGAACAGGAUCUG
CUGGCACUGGACUGAUAACUCGAG
BG505_SOSIP_MD39_link14 RNA
GGAUCCGCCACCAUGGACUGGACAUGGAUUCUGUU
CCUGGUCGCUGCCGCUACAAGAGUGCAUUCCGCCG
AAAACCUGUGGGUCACCGUCUACUAUGGAGUGCCC
GUGUGGAAGGACGCCGAGACUACGCUGUUCUGCGC
CAGCGAUGCCAAGGCCUACGAGACAGAGAAGCACA
ACGUGUGGGCAACCCACGCAUGCGUGCCUACAGAC
CCAAACCCCCAGGAGAUCCACCUGGAGAAUGUGAC
AGAGGAGUUUAACAUGUGGAAGAACAAUAUGGUGG
AGCAGAUGCACGAGGACAUCAUCUCCCUGUGGGAU
CAGUCUCUGAAGCCCUGCGUGAAGCUGACCCCUCU
GUGCGUGACACUGCAGUGUACCAACGUGACAAACA
AUAUCACCGACGAUAUGCGGGGCGAGCUGAAGAAU
UGUAGCUUCAACAUGACCACAGAGCUGAGGGACAA
GAAGCAGAAGGUGUACUCCCUGUUUUAUAGACUGG
AUGUGGUGCAGAUCAAUGAGAACCAGGGCAAUCGG
UCUAACAAUAGCAACAAGGAGUACCGCCUGAUCAA
UUGCAACACCUCCGCCAUCACACAGGCCUGUCCUA
AGGUGUCUUUCGAGCCUAUCCCAAUCCACUAUUGC
GCCCCAGCCGGCUUCGCCAUCCUGAAGUGUAAGGA
UAAGAAGUUUAACGGAACCGGACCAUGCCCUUCCG
UGUCUACCGUGCAGUGUACACACGGCAUCAAGCCU
GUGGUGUCUACACAGCUGCUGCUGAAUGGCAGCCU
GGCCGAGGAGGAAGUGAUCAUCAGGUCUGAGAACA
UCACCAACAAUGCCAAGAAUAUCCUGGUGCAGCUG
AACACACCAGUGCAGAUCAAUUGCACCCGGCCCAA
CAAUAACACAGUGAAGUCUAUCCGCAUCGGCCCAG
GCCAGGCCUUUUACUAUACCGGCGACAUCAUCGGC
GAUAUCAGACAGGCCCACUGUAAUGUGAGCAAGGC
CACCUGGAACGAGACACUGGGCAAGGUGGUGAAGC
AGCUGAGGAAGCACUUCGGCAAUAACACCAUCAUC
AGAUUUGCACAGAGCUCCGGCGGCGACCUGGAGGU
GACCACACACUCCUUCAAUUGCGGCGGCGAGUUCU
UUUACUGUAACACAAGCGGCCUGUUUAAUUCCACC
UGGAUCUCCAACACAUCUGUGCAGGGCAGCAAUUC
CACCGGCAGCAACGAUUCCAUCACACUGCCAUGCC
GGAUCAAGCAGAUCAUCAACAUGUGGCAGCGCAUC
GGCCAGGCCAUGUAUGCCCCUCCCAUCCAGGGCGU
GAUCAGAUGCGUGAGCAAUAUCACCGGCCUGAUCC
UGACACGCGACGGCGGCUCUACCAACAGCACCACA
GAGACAUUCCGGCCCGGCGGCGGCGACAUGAGGGA
UAACUGGAGAUCUGAGCUGUACAAGUAUAAGGUGG
UGAAGAUCGAGCCUCUGGGAGUGGCACCAACCAGG
UGCAAGAGGAGAGUGGUGGGCUCUCACAGCGGCUC
CGGCGGCUCUGGCAGCGGCGGCCACGCCGCAGUGG
GCAUCGGAGCCGUGUCCCUGGGCUUUCUGGGAGCA
GCAGGCUCCACAAUGGGAGCAGCCUCUAUGACCCU
GACAGUGCAGGCCAGGAAUCUGCUGAGCGGCAUCG
UGCAGCAGCAGUCCAACCUGCUGAGAGCCCCAGAG
CCCCAGCAGCACCUGCUGAAGGACACCCACUGGGG
CAUCAAGCAGCUGCAGGCCAGGGUGCUGGCAGUGG
AGCACUAUCUGAGAGAUCAGCAGCUGCUGGGCAUC
UGGGGCUGUAGCGGCAAGCUGAUCUGCUGUACCAA
UGUGCCCUGGAACUCUAGCUGGUCUAAUCGCAACC
UGAGCGAGAUCUGGGACAAUAUGACCUGGCUGCAG
UGGGAUAAGGAGAUCUCCAACUACACACAGAUCAU
CUAUGGCCUGCUGGAAGAAUCUCAGAAUCAGCAGG
AAAAGAAUGAACAGGAUCUGCUGGCACUGGAUUGA
UAACUCGAG
BG505_SOSIP_MD39_CPG9.2_
circular permutation)-RNA
(SEQ ID NO: 263)
GGAUCCGCCACCAUGGAUUGGACUUGGAUUCUGUU
CCUGGUCGCAGCAGCCACACGAGUGCAUAGCGGGG
GAAAUAGUAGCGGCAGCCUGGGGUUCCUGGGAGCA
GCAGGCUCCACCAUGGGAGCAGCAUCUAUGACCCU
GACAGUGCAGGCCAGGAAUCUGCUGUCUGGCAUCG
UGCAGCAGCAGAGCAACCUGCUGAGAGCCCCAGAG
CCCCAGCAGCACCUGCUGAAGGACACCCACUGGGG
CAUCAAGCAGCUGCAGGCCCGGGUGCUGGCAGUGG
AGCACUACCUGCGCGAUCAGCAGCUGCUGGGAAUC
UGGGGAUGCAGCGGCAAGCUGAUCUGCUGUACAAA
UGUGCCUUGGAACAGCUCCUGGUCCAAUAGGAACC
UGUCUGAGAUCUGGGACAAUAUGACCUGGCUGAAC
UGGUCUAAGGAGAUCAGCAAUUACACACAGAUCAU
CUAUGGCCUGCUGGAGGAGAGCCAGAAUCAGAACG
AGUCCAAUGAGCAGGAUCUGGGCGGCAACGGCAGC
GGCGGCGGCAGCGGCUCCGGCGGCAACGGCUCUAG
CGGCCUGUGGGUGACCGUGUACUAUGGCGUGCCCG
UGUGGAAGGACGCCGAGACUACGCUGUUCUGCGCC
UCCGAUGCCAAGGCCUAUGAGACAGAGAAGCACAA
CGUGUGGGCAACCCACGCAUGCGUGCCAACAGACC
CUAACCCACAGGAGAUCCACCUGGAGAAUGUGACC
GAGGAGUUUAACAUGUGGAAGAACAAUAUGGUGGA
GCAGAUGCACGAGGACAUCAUCAGCCUGUGGGAUC
AGUCCCUGAAGCCUUGCGUGAAGCUGACCCCACUG
UGCGUGACACUGCAGUGUACCAACGUGACAAACAA
UAUCACCGACGAUAUGAGGGGCGAGCUGAAGAAUU
GUUCUUUCAACAUGACCACAGAGCUGAGGGACAAG
AAGCAGAAAGUGUACAGCCUGUUUUAUAGACUGGA
UGUGGUGCAGAUCAAUGAGAACCAGGGCAAUAGGA
GCAACAAUUCCAACAAGGAGUACAGACUGAUCAAU
UGCAACACCAGCGCCAUCACACAGGCCUGUCCAAA
GGUGUCCUUCGAGCCCAUCCCUAUCCACUAUUGCG
CACCAGCAGGAUUCGCAAUCCUGAAGUGUAAGGAU
AAGAAGUUUAACGGAACCGGACCAUGCCCAUCUGU
GAGCACCGUGCAGUGUACACACGGCAUCAAGCCAG
UGGUGUCCACACAGCUGCUGCUGAAUGGCUCUCUG
GCCGAGGAGGAAGUGAUCAUCCGGAGCGAGAACAU
CACCAACAAUGCCAAGAAUAUCCUGGUGCAGCUGA
ACACACCCGUGCAGAUCAAUUGCACCCGGCCUAAC
AAUAACACAGUGAAGUCCAUCAGGAUCGGACCAGG
ACAGGCCUUUUACUAUACCGGCGACAUCAUCGGCG
AUAUCCGCCAGGCCCACUGUAACGUGAGCAAGGCC
ACCUGGAACGAGACACUGGGCAAGGUGGUGAAGCA
GCUGAGGAAGCACUUCGGCAAUAACACCAUCAUCA
GAUUUGCACAGUCCUCUGGCGGCGACCUGGAGGUG
ACCACACACUCCUUCAACUGCGGCGGCGAGUUCUU
UUACUGUAACACAUCUGGCCUGUUUAAUAGCACCU
GGAUCUCUAACACAAGCGUGCAGGGCUCCAAUUCU
ACCGGCUCCAACGAUUCUAUCACACUGCCCUGCCG
GAUCAAGCAGAUCAUCAACAUGUGGCAGAGGAUCG
GACAGGCAAUGUACGCCCCUCCCAUCCAGGGCGUG
AUCAGAUGCGUGAGCAAUAUCACCGGCCUGAUCCU
GACACGCGACGGCGGCAGCACCAACUCCACCACAG
AGACAUUCAGACCCGGCGGCGGCGACAUGAGGGAU
AACUGGAGAUCCGAGCUGUAUAAGUAUAAAGUCGU
GAAGAUUGAGCCACUGGGCGUCGCACCAACAAGAU
GUAAUAGAAGCUGAUAACUCGAG
BG505_MD39_GRSF (Glycan)-RNA
(SEQ ID NO: 264)
GGAUCCGCCACCAUGGACUGGACAUGGAUUCUGUU
CCUGGUCGCUGCCGCUACAAGAGUGCAUUCCGCCG
AAAACCUGUGGGUCACCGUCUACUAUGGAGUGCCC
GUGUGGAAGGACGCCGAGACUACGCUGUUCUGCGC
CAGCGAUGCCAAGGCCUACGAGACAGAGAAGCACA
ACGUGUGGGCAACCCACGCAUGCGUGCCUACAGAC
CCAAACCCCCAGGAGAUCCACCUGGAGAAUGUGAC
AGAGGAGUUUAACAUGUGGAAGAACAAUAUGGUGG
AGCAGAUGCACGAGGACAUCAUCUCCCUGUGGGAU
CAGUCUCUGAAGCCCUGCGUGAAGCUGACCCCUCU
GUGCGUGACACUGCAGUGUACCAACGUGACAAACA
AUAUCACCGACGAUAUGCGGGGCGAGCUGAAGAAU
UGUAGCUUCAACAUGACCACAGAGCUGAGGGACAA
GAAGCAGAAGGUGUACUCCCUGUUUUAUAGACUGG
AUGUGGUGCAGAUCAAUGAGAACCAGGGCAAUCGG
UCUAACAAUAGCAACAAGGAGUACCGCCUGAUCAA
UUGCAACACCUCCGCCAUCACACAGGCCUGUCCUA
AGGUGUCUUUCGAGCCUAUCCCAAUCCACUAUUGC
GCCCCAGCCGGCUUCGCCAUCCUGAAGUGUAAGGA
UAAGAAGUUUAACGGAACCGGACCAUGCCCUUCCG
UGUCUACCGUGCAGUGUACACACGGCAUCAAGCCU
GUGGUGUCUACACAGCUGCUGCUGAAUGGCAGCCU
GGCCGAGGAGGAAGUGAUCAUCAGGUCUGAGAACA
UCACCAACAAUGCCAAGAAUAUCCUGGUGCAGCUG
AACACACCAGUGCAGAUCAAUUGCACCCGGCCCAA
CAAUAACACAGUGAAGUCUAUCCGCAUCGGCCCAG
GCCAGGCCUUUUACUAUACCGGCGACAUCAUCGGC
GAUAUCAGACAGGCCCACUGUAAUGUGAGCAAGGC
CACCUGGAACGAGACACUGGGCAAGGUGGUGAAGC
AGCUGAGGAAGCACUUCGGCAAUAACACCAUCAUC
AGAUUUGCACAGAGCUCCGGCGGCGACCUGGAGGU
GACCACACACUCCUUCAAUUGCGGCGGCGAGUUCU
UUUACUGUAACACAAGCGGCCUGUUUAAUUCCACC
UGGAUCUCCAACACAUCUGUGCAGGGCAGCAAUUC
CACCGGCAGCAACGAUUCCAUCACACUGCCAUGCC
GGAUCAAGCAGAUCAUCAACAUGUGGCAGCGCAUC
GGCCAGGCCAUGUAUGCCCCUCCCAUCCAGGGCGU
GAUCAGAUGCGUGAGCAAUAUCACCGGCCUGAUCC
UGACACGCGACGGCGGCUCUACCAACAGCACCACA
GAGACAUUCCGGCCCGGCGGCGGCGACAUGAGGGA
UAACUGGAGAUCUGAGCUGUACAAGUAUAAGGUGG
UGAAGAUCGAGCCUCUGGGAGUGGCACCAACCAGG
UGCAAGAGGAGAGUGGUGGGCCGGCGCAGGAGACG
GCGCGCAGUGGGCAUCGGAGCCGUGUCCCUGGGCU
UUCUGGGAGCAGCAGGCUCCACAAUGGGAGCAGCC
UCUAUGACCCUGACAGUGCAGGCCAGGAAUCUGCU
GAGCGGCAUCGUGCAGCAGCAGUCCAACCUGCUGA
GAGCCCCAGAGCCCCAGCAGCACCUGCUGAAGGAC
ACCCACUGGGGCAUCAAGCAGCUGCAGGCCAGGGU
GCUGGCAGUGGAGCACUAUCUGAGAGAUCAGCAGC
UGCUGGGCAUCUGGGGCUGUAGCGGCAAGCUGAUC
UGCUGUACCAAUGUGCCCUGGAACUCUAGCUGGUC
UAAUCGCAACCUGAGCGAGAUCUGGGACAAUAUGA
CCUGGCUGAACUGGAGCAAGGAGAUCUCCAACUAC
ACACAGAUCAUCUAUGGCCUGCUGGAAGAAUCUCA
GAAUCAGCAGGAAAAGAAUAACCAGAGCCUGCUGG
CACUGGAUUGAUAACUCGAG
BG505_SOSIP_MD39-RNA
(SEQ ID NO: 265)
GGAUCCGCCACCAUGGACUGGACAUGGAUUCUGUU
CCUGGUCGCUGCCGCUACAAGAGUGCAUUCCGCCG
AAAACCUGUGGGUCACCGUCUACUAUGGAGUGCCC
GUGUGGAAGGACGCCGAGACUACGCUGUUCUGCGC
CAGCGAUGCCAAGGCCUACGAGACAGAGAAGCACA
ACGUGUGGGCAACCCACGCAUGCGUGCCUACAGAC
CCAAACCCCCAGGAGAUCCACCUGGAGAAUGUGAC
AGAGGAGUUUAACAUGUGGAAGAACAAUAUGGUGG
AGCAGAUGCACGAGGACAUCAUCUCCCUGUGGGAU
CAGUCUCUGAAGCCCUGCGUGAAGCUGACCCCUCU
GUGCGUGACACUGCAGUGUACCAACGUGACAAACA
AUAUCACCGACGAUAUGCGGGGCGAGCUGAAGAAU
UGUAGCUUCAACAUGACCACAGAGCUGAGGGACAA
GAAGCAGAAGGUGUACUCCCUGUUUUAUAGACUGG
AUGUGGUGCAGAUCAAUGAGAACCAGGGCAAUCGG
UCUAACAAUAGCAACAAGGAGUACCGCCUGAUCAA
UUGCAACACCUCCGCCAUCACACAGGCCUGUCCUA
AGGUGUCUUUCGAGCCUAUCCCAAUCCACUAUUGC
GCCCCAGCCGGCUUCGCCAUCCUGAAGUGUAAGGA
UAAGAAGUUUAACGGAACCGGACCAUGCCCUUCCG
UGUCUACCGUGCAGUGUACACACGGCAUCAAGCCU
GUGGUGUCUACACAGCUGCUGCUGAAUGGCAGCCU
GGCCGAGGAGGAAGUGAUCAUCAGGUCUGAGAACA
UCACCAACAAUGCCAAGAAUAUCCUGGUGCAGCUG
AACACACCAGUGCAGAUCAAUUGCACCCGGCCCAA
CAAUAACACAGUGAAGUCUAUCCGCAUCGGCCCAG
GCCAGGCCUUUUACUAUACCGGCGACAUCAUCGGC
GAUAUCAGACAGGCCCACUGUAAUGUGAGCAAGGC
CACCUGGAACGAGACACUGGGCAAGGUGGUGAAGC
AGCUGAGGAAGCACUUCGGCAAUAACACCAUCAUC
AGAUUUGCACAGAGCUCCGGCGGCGACCUGGAGGU
GACCACACACUCCUUCAAUUGCGGCGGCGAGUUCU
UUUACUGUAACACAAGCGGCCUGUUUAAUUCCACC
UGGAUCUCCAACACAUCUGUGCAGGGCAGCAAUUC
CACCGGCAGCAACGAUUCCAUCACACUGCCAUGCC
GGAUCAAGCAGAUCAUCAACAUGUGGCAGCGCAUC
GGCCAGGCCAUGUAUGCCCCUCCCAUCCAGGGCGU
GAUCAGAUGCGUGAGCAAUAUCACCGGCCUGAUCC
UGACACGCGACGGCGGCUCUACCAACAGCACCACA
GAGACAUUCCGGCCCGGCGGCGGCGACAUGAGGGA
UAACUGGAGAUCUGAGCUGUACAAGUAUAAGGUGG
UGAAGAUCGAGCCUCUGGGAGUGGCACCAACCAGG
UGCAAGAGGAGAGUGGUGGGCCGGCGCAGGAGACG
GCGCGCAGUGGGCAUCGGAGCCGUGUCCCUGGGCU
UUCUGGGAGCAGCAGGCUCCACAAUGGGAGCAGCC
UCUAUGACCCUGACAGUGCAGGCCAGGAAUCUGCU
GAGCGGCAUCGUGCAGCAGCAGUCCAACCUGCUGA
GAGCCCCAGAGCCCCAGCAGCACCUGCUGAAGGAC
ACCCACUGGGGCAUCAAGCAGCUGCAGGCCAGGGU
GCUGGCAGUGGAGCACUAUCUGAGAGAUCAGCAGC
UGCUGGGCAUCUGGGGCUGUAGCGGCAAGCUGAUC
UGCUGUACCAAUGUGCCCUGGAACUCUAGCUGGUC
UAAUCGCAACCUGAGCGAGAUCUGGGACAAUAUGA
CCUGGCUGCAGUGGGAUAAGGAGAUCUCCAACUAC
ACACAGAUCAUCUAUGGCCUGCUGGAAGAAUCUCA
GAAUCAGCAGGAAAAGAAUGAACAGGAUCUGCUGG
CACUGGAUUGAUAACUCGAG

### B. Polypeptide Sequences

Disclosed are the polypeptide sequences encoded by the disclosed nucleic acid sequences. Thus, disclosed are the polypeptide sequences encoded by the leader sequence, self-assembling polypeptide encoded by a nucleotide sequence, polypeptide sequences encoded by the linker, and viral antigens encoded by a nucleotide sequence. The disclosure also relates to cells expressing one or more polypeptides disclosed in the application.

In some embodiments, the polypeptide encoded by the leader sequence can be the IgE amino acid sequence MDWTWILFLVAAATRVHS encoded by SEQ ID NO:1-6.

MQIYEGKLTAEGLRFGIVASRANHALVDRLVEGAIDAIVRH
GGREEDITLVRVCGSWEIPVAAGELARKEDIDAVIAIGVLC
RGATPSFDYIASEVSKGLADLSLELRKPITFGVITADTLEQ
AIEAAGTCHGNKGWEAALCAIEMANLFKSLRGGS
encoded by SEQ ID NO:.

In some embodiments, the polypeptide sequences encoded by a portion of the expressible nucleic acid sequence can be GGSGGSGGSGGG.

Also disclosed is the polypeptide comprising the IgE leader sequence and a gp120 variant viral antigen comprising the sequence MDWTWILFLVAAATRVHSDTITLPCRPAPPPHCSSNITGLILTRQGGYSNDNTVIFRPS GGDWRDIARCQIAGTVVSTQLFLNGSLAEEEVVIRSEDWRDNAKSICVQLNTSVEIN CTGAGHCNISRAKWNNTLKQIASKLREQYGNKTIIFKPSSGGDPEFVNHSFNCGGEFF YCDSTQLFNSTWFNST. In some embodiments, the composition comprises at least one expressible nucleic acid sequence disclosed herein or any nucleic acid sequence at least about 70%, 75%, 80%, 85%, 86%, 87% 88%, 89%, 90%, 91%, 92%, 93%, 94%, 95%, 96%, 97%, 98%, 99%, or 100% sequence identity to SEQ ID NO: 53, SEQ ID NO: 54, SEQ ID NO: 56, SEQ ID NO: 57, SEQ ID NO: 59 SEQ ID NO: 60, SEQ ID NO: 62 SEQ ID NO: 63, SEQ ID NO: 78, SEQ ID NO: 79, SEQ ID NO: 81, SEQ ID NO: 82, SEQ ID NO: 84, SEQ ID NO: 85, SEQ ID NO: 87, SEQ ID NO: 88, SEQ ID NO: 90, SEQ ID NO: 91 or a pharmaceutically acceptable salt of any of the foregoing. In some embodiments, the composition comprises at least one expressible nucleic acid sequence disclosed herein or any nucleic acid sequence at least about 70%, 75%, 80%, 85%, 86%, 87% 88%, 89%, 90%, 91%, 92%. 93%, 94%, 95%, 96%, 97%, 98%, 99%, or 100% sequence identity to SEQ ID NO: 106 SEQ ID NO: 107, SEQ ID NO: 109, SEQ ID NO: 110, SEQ ID NO: 112, SEQ ID NO: 113, SEQ ID NO: 115, SEQ ID NO: 116, SEQ ID NO: 118, SEQ ID NO: 119, SEQ ID NO: 121, SEQ ID NO: 122, SEQ ID NO: 124, SEQ ID NO: 125, SEQ ID NO: 127, SEQ ID NO: 128, SEQ ID NO: 130, SEQ ID NO: 131 or a pharmaceutically acceptable salt of any of the foregoing.

In some embodiments, the composition, nucleic acid molecule or nucleic acid sequence of the disclosure relates to any a plasmid comprising any nucleic acid or combination of nucleic acid sequences chosen from those that are at least about 70%, 75%, 80%, 85%, 86%, 87% 88%, 89%, 90%, 91%, 92%, 93%, 94%, 95%, 96%, 97%, 98%, 99%, or 100% sequence identity to those nucleic acid sequences disclosed from SEQ ID NO: 154 through SEQ ID NO: 238. In some embodiments, the composition, nucleic acid molecule or nucleic acid sequence of the disclosure relates to any a plasmid comprising any nucleic acid or combination of nucleic acid sequences that encode an amino acid sequence that comprises at least about 70%, 75%, 80%, 85%, 86%, 87% 88%, 89%, 90%, 91%, 92%, 93%, 94%, 95%, 96%, 97%, 98%, 99%, or 100% sequence identity to any amino acid sequence within or between from SEQ ID NO: 154 through SEQ ID NO: 238.

### C. Pharmaceutical Compositions

Disclosed are pharmaceutical compositions comprising any one or more of the disclosed compositions and a pharmaceutically acceptable carrier.

In some embodiments, any of the disclosed compositions is from about 1 to about 30 micrograms of the disclosed DNA and/or RNA vaccine. For example, any of the disclosed compositions can be from about 1 to about 5 micrograms the disclosed DNA and/or RNA vaccine. In some preferred embodiments, the pharmaceutical compositions contain from about 5 nanograms to about 800 micrograms of the disclosed DNA and/or RNA vaccine. In some preferred embodiments, the pharmaceutical compositions contain about 25 to about 250 micrograms, from about 100 to about 200 micrograms, from about 1 nanogram to 100 milligrams; from about 1 microgram to about 10 milligrams; from about 0.1 microgram to about 10 milligrams; from about 1 milligram to about 2 milligrams, from about 5 nanograms to about 1000 micrograms, from about 10 nanograms to about 800 micrograms, from about 0.1 to about 500 micrograms, from about 1 to about 350 micrograms, from about 25 to about 250 micrograms, from about 100 to about 200 micrograms of the DNA and/or RNA vaccine or plasmid thereof. The pharmaceutical compositions can comprise from about 5 nanograms to about 10 mg of the disclosed DNA and/or RNA vaccine. In some embodiments, pharmaceutical compositions according to the present invention comprise from about 25 nanograms to about 5 mg of the disclosed DNA and/or RNA vaccine. In some embodiments, the pharmaceutical compositions contain from about 50 nanograms to about 1 mg of the disclosed DNA and/or RNA vaccine. In some embodiments, the pharmaceutical compositions contain about from about 0.1 to about 500 micrograms of the disclosed DNA and/or RNA vaccine. In some embodiments, the pharmaceutical compositions contain from about 1 to about 350 micrograms of the disclosed DNA and/or RNA vaccine. In some embodiments, the pharmaceutical compositions contain from about 5 to about 250 micrograms of the disclosed DNA and/or RNA vaccine. In some embodiments, the pharmaceutical compositions contain from about 10 to about 200 micrograms of the disclosed DNA and/or RNA vaccine. In some embodiments, the pharmaceutical compositions contain from about 15 to about 150 micrograms of the disclosed DNA and/or RNA vaccine. In some embodiments, the pharmaceutical compositions contain about 20 to about 100 micrograms of the disclosed DNA and/or RNA vaccine. In some embodiments, the pharmaceutical compositions contain about 25 to about 75 micrograms of the disclosed DNA and/or RNA vaccine. In some embodiments, the pharmaceutical compositions contain about 30 to about 50 micrograms of the disclosed DNA and/or RNA vaccine. In some embodiments, the pharmaceutical compositions contain about 35 to about 40 micrograms of the disclosed DNA and/or RNA vaccine. In some embodiments, the pharmaceutical compositions contain about 100 to about 200 micrograms the disclosed DNA and/or RNA vaccine. In some embodiments, the pharmaceutical compositions comprise about 10 micrograms to about 100 micrograms of the disclosed DNA and/or RNA vaccine. In some embodiments, the pharmaceutical compositions comprise about 20 micrograms to about 80 micrograms of the disclosed DNA and/or RNA vaccine. In some embodiments, the pharmaceutical compositions comprise about 25 micrograms to about 60 micrograms of the disclosed DNA and/or RNA vaccine. In some embodiments, the pharmaceutical compositions comprise about 30 nanograms to about 50 micrograms of the disclosed DNA and/or RNA vaccine. In some embodiments, the pharmaceutical compositions comprise about 35 nanograms to about 45 micrograms of the disclosed DNA and/or RNA vaccine. In some preferred embodiments, the pharmaceutical compositions contain about 0.1 to about 500 micrograms of the disclosed DNA and/or RNA vaccine. In some preferred embodiments, the pharmaceutical compositions contain about 1 to about 350 micrograms of the disclosed DNA and/or RNA vaccine. In some preferred embodiments, the pharmaceutical compositions contain about 1 to about 250 micrograms of the disclosed DNA and/or RNA vaccine. In some preferred embodiments, the pharmaceutical compositions contain about 2 to about 200 micrograms the disclosed DNA and/or RNA vaccine.

In some embodiments, pharmaceutical compositions according to the present invention comprise at least about 10, 15, 20, 25, 30, 35, 40, 45, 50, 55, 60, 65, 70, 75, 80, 85, 90, 95 or 100 nanograms of the disclosed DNA and/or RNA vaccine. In some embodiments, the pharmaceutical compositions can comprise at least about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830,835, 840,845, 850, 855, 860, 865,870, 875, 880, 885, 890, 895, 900, 905, 910, 915, 920, 925, 930, 935, 940, 945, 950, 955, 960, 965, 970, 975, 980, 985, 990, 995 or 1000 micrograms of the disclosed DNA and/or RNA vaccine. In some embodiments, the pharmaceutical composition can comprise at least 1.5, 2, 2.5, 3, 3.5, 4, 4.5, 5, 5.5, 6, 6.5, 7, 7.5, 8, 8.5, 9, 9.5 or 10 mg or more of the disclosed DNA and/or RNA vaccine.

In other embodiments, the pharmaceutical composition can comprise up to and including about 10, 15, 20, 25, 30, 35, 40, 45, 50, 55, 60, 65, 70, 75, 80, 85, 90, 95 or 100 nanograms of the disclosed DNA and/or RNA vaccine. In some embodiments, the pharmaceutical composition can comprise up to and including about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 micrograms of the disclosed DNA and/or RNA vaccine. In some embodiments, the pharmaceutical composition can comprise up to and including about 1.5, 2, 2.5, 3, 3.5, 4, 4.5, 5, 5.5, 6, 6.5, 7, 7.5, 8, 8.5, 9, 9.5 or about 10 mg of the disclosed DNA and/or RNA vaccine. The pharmaceutical composition can further comprise other agents for formulation purposes according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The vaccine can further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or other known transfection facilitating agents. In some embodiments, the vaccine is a composition comprising a plasmid DNA molecule, RNA molecule or DNA/RNA hybrid molecule encoding an expressible nucleic acid sequence, the expressible nucleic acid sequence comprising a first nucleic acid encoding a self-assembling nanoparticle polypeptide and a second nucleic acid sequence comprising one, two, or three or more contiguous or non-contiguous retroviral envelope antigens, optionally encoding a leader sequence disclosed herein.

The transfection facilitating agent is a polyanion, polycation, including poly-L-glutamate (LGS), or lipid. The transfection facilitating agent is poly-L-glutamate, and more preferably, the poly-L-glutamate is present in the vaccine at a concentration less than 6 mg/ml. The transfection facilitating agent can also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid can also be used administered in conjunction with the genetic construct. In some embodiments, the DNA vector vaccines can also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant. The adjuvant can be other genes that are expressed in alternative plasmid or are deneurological systemed as proteins in combination with the plasmid above in the vaccine. The adjuvant can be selected from the group consisting of α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof. In an exemplary embodiment, the adjuvant is IL-12.

Other genes which can be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof or a combination thereof.

In some embodiments adjuvant may be one or more proteins and/or nucleic acid molecules that encode proteins selected from the group consisting of: CCL-20, IL-12, IL-15, IL-28, CTACK, TECK, MEC or RANTES. Examples of IL-12 constructs and sequences are disclosed in PCT application No. PCT/US1997/019502 (published as WO98/017799) and corresponding U.S. application Ser. No. 08/956,865, and U.S. Provisional Application No. 61/569,600 filed Dec. 12, 2011, which are each incorporated herein by reference in their entireties. Examples of IL-15 constructs and sequences are disclosed in PCT application No. PCT/US04/18962 (published as WO2005/000235) and corresponding U.S. application Ser. No. 10/560,650, and in PCT application No. PCT/US07/00886 (published as WO2007/087178) and corresponding U.S. application Ser. No. 12/160,766, and in PCT Application Serial No. PCT/US10/048827 (published as WO2011/032179), which are each incorporated herein by reference in their entireties. Examples of IL-28 constructs and sequences are disclosed in PCT application no. PCT/US09/039648 (published as WO2009/124309) and corresponding U.S. application Ser. No. 12/936,192, which are each incorporated herein by reference in their entireties. Examples of RANTES and other constructs and sequences are disclosed in PCT application No. PCT/US 1999/004332 (published as WO99/043839) and corresponding U.S. Application Serial No. and 09/622,452, which are each incorporated herein by reference in their entireties. Other examples of RANTES constructs and sequences are disclosed in PCT Application No. PCT/US Serial No. 11/024098 (published as WO2011/097640), which is incorporated herein by reference. Examples of RANTES and other constructs and sequences are disclosed in PCT Application No. PCT/US 1999/004332 and corresponding U.S. application Ser. No. 09/622,452, which are each incorporated herein by reference. Other examples of RANTES constructs and sequences are disclosed in PCT application No. PCT/US11/024098 (published as WO2011/097640), which is incorporated herein by reference in its entirety. Examples of chemokines CTACK, TECK and MEC constructs and sequences are disclosed in PCT Application No. PCT/US2005/042231 (published as WO2007/050095) and corresponding U.S. application Ser. No. 11/719,646, which are each incorporated herein by reference in their entireties. Examples of OX40 and other immunomodulators are disclosed in U.S. application Ser. No. 10/560,653, which is incorporated herein by reference in its entirety. Examples of DR5 and other immunomodulators are disclosed in U.S. application Ser. No. 09/622,452, which is incorporated herein by reference in its entirety.

The pharmaceutical composition may be formulated according to the mode of administration to be used. An injectable vaccine pharmaceutical composition may be sterile, pyrogen free and particulate free. An isotonic formulation or solution may be used. Additives for isotonicity may include sodium chloride, dextrose, mannitol, sorbitol, and lactose. The vaccine may comprise a vasoconstriction agent. The isotonic solutions may include phosphate buffered saline. Vaccine may further comprise stabilizers including gelatin and albumin. The stabilizing may allow the formulation to be stable at room or ambient temperature for extended periods of time such as LGS or polycations or polyanions to the vaccine formulation.

The vaccine can be a DNA vaccine. DNA vaccines are disclosed in U.S. Pat. Nos. 5,593,972, 5,739,118, 5,817,637, 5,830,876, 5,962,428, 5,981,505, 5,580,859, 5,703,055, and 5,676,594, which are incorporated herein fully by reference. The DNA vaccine can further comprise elements or reagents that inhibit it from integrating into the chromosome. Examples of attenuated live vaccines, those using recombinant vectors to foreign antigens, subunit vaccines and glycoprotein vaccines are described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference in their entireties.

The genetic construct can also be part of a genome of a recombinant viral vector, including recombinant adenovirus, recombinant adenovirus associated virus and recombinant vaccinia. The genetic construct can be part of the genetic material in attenuated live microorganisms or recombinant microbial vectors which live in cells

### D. Methods

Disclosed are methods of vaccinating a subject comprising administering a therapeutically effective amount of any of the disclosed pharmaceutical compositions to the subject. Disclosed are methods of inducing an immune response in a subject comprising administering to the subject any of the disclosed pharmaceutical compositions.

Disclosed are methods of neutralizing one or a plurality of viruses in a subject comprising administering to the subject any of the disclosed pharmaceutical compositions.

Disclosed are methods of stimulating a therapeutically effective antigen-specific immune response against a virus in a mammal infected with the virus comprising administering any of the disclosed pharmaceutical compositions. Disclosed are methods of inducing expression of a self-assembling vaccine in a subject comprising administering any of the disclosed pharmaceutical compositions. Also disclosed are methods of treating a subject having a viral infection or susceptible to becoming infected with a virus comprising administering to the subject any of the disclosed pharmaceutical compositions.

In some embodiments, the administering can be accomplished by oral administration, parenteral administration, sublingual administration, transdermal administration, rectal administration, transmucosal administration, topical administration, inhalation, buccal administration, intrapleural administration, intravenous administration, intraarterial administration, intraperitoneal administration, subcutaneous administration, intramuscular administration, intranasal administration, intrathecal administration, and intraarticular administration, or combinations thereof. In some embodiments, the above modes of action are accomplished by injection of the pharmaceutical compositions disclosed herein. In some embodiments, the therapeutically effective dose can be from about 1 to about 30 micrograms of expressible nucleic acid sequence. In some embodiments, the therapeutically effective dose can be from about 0.001 micrograms of composition per kilogram of subject to about 0.050 micrograms per kilogram of subject.

In some embodiments, any of the disclosed methods can be free of activating any mannose-binding lectin or complement process.

In some embodiments, the subject can be a human. In some embodiments, the subject is diagnosed with or suspected of having a viral infection. For example, the subject can be diagnosed with or suspected of having an HIV-1 infection.

In some embodiments of the methods of inducing an immune response, the immune response can be an antigen-specific immune response. For example, the antigen-specific immune response can be an HIV-1 antigen immune response.

In some embodiments, any of the disclosed methods can further comprise administering to the subject a pharmaceutical composition comprising one or more pharmaceutically active agents, such as antiviral drugs, among many others. In some embodiments, the one or more pharmaceutically active agents include other antiretroviral medications used to inhibit HIV, for example nucleoside analog reverse transcriptase inhibitors, non-nucleoside reverse transcriptase inhibitors, and protease inhibitors. Among the available drugs that may be used as a pharmaceutically active agent are zidovudine or AZT (or Retrovir®), didanosine or DDI (or Videx®), stavudine or D4T (or Zerit®), lamivudine or 3TC (or EpivirR), zalcitabine or DDC (or Hivid®), abacavir succinate (or Ziagen”), tenofovir disoproxil fumarate salt (or Viread®), emtricitabine (or Emtriva®), Combivir® (contains 3TC and AZT). Trizivir® (contains abacavir, 3TC and AZT); three non-nucleoside reverse transcriptase inhibitors: nevirapine (or Viramune®), delavirdine (or Rescriptor®) and efavirenz (or Sustiva®), eight peptidomimetic protease inhibitors or approved formulations: saquinavir (or InviraseR or Fortovase”), indinavir (or Crixivan®), ritonavir (or Norvir®), nelfinavir (or Viracept”), amprenavir (or Agenerase®), atazanavir (Reyataz), fosamprenavir (or Lexiva), Kaletra® (contains lopinavir and ritonavir), and one fusion inhibitor enfuvirtide (or T-20 or FuzeonR).

In some embodiments, the methods are free of administering any polypeptide directly to the subject. In some embodiments, methods of inducing an immune response can include inducing a humoral or cellular immune response. A humoral immune response can include induction of CD4+ cells and antibody production. A cellular immune response can include activating CD8+ cells and cytotoxic activity. In one aspect, the present disclosure features a method of inducing an immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein. In one aspect, the present disclosure features a method of inducing a CD8+ T cell immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein.

In one aspect, the present disclosure features a method of enhancing an immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein.

In one aspect, the present disclosure features a method of enhancing a CD8+ T cell immune response in a subject against a virus, the method comprising administering to the subject in need thereof a pharmaceutically effective amount of any of the nucleic acid molecules of any one of the aspects or embodiments herein, or any one of the pharmaceutical compositions of any one of the aspects and embodiments herein. In another embodiment, the subject has previously been treated, and not responded to anti-viral therapy. In some embodiments, the nucleic acid molecule and/or expressible sequence is administered to the subject by electroporation.

The nucleic acid sequence or vaccine may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The vaccine may be administered by traditional syringes, needleless injection devices, “microprojectile bombardment gone guns”, or other physical methods such as electroporation (“EP”), “hydrodynamic method”, or ultrasound.

The plasmid comprising one, two three or more expressible nucleic acid sequences may be delivered to the mammal by several well-known technologies including DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. The consensus antigen may be delivered via DNA injection and, optionally, with in vivo electroporation. In some embodiments, the vaccine or pharmaceutical composition can be administered by electroporation. Administration of the vaccine via electroporation of the plasmids of the vaccine may be accomplished using electroporation devices that can be configured to deliver to a desired tissue of a mammal a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation can be accomplished using an in vivo electroporation device, for example CELLECTRA® EP system (Inovio Pharmaceuticals, Inc., Blue Bell, Pa.) or Elgen electroporator (Inovio Pharmaceuticals, Inc.) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance. The feedback mechanism may be performed by either hardware or software. The feedback mechanism may be performed by an analog closed-loop circuit. The feedback occurs every 50 μs, 20 s, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the DNA vaccines of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the DNA vaccines include those provided in co-pending and co-owned U.S. patent application Ser. No. 11/874,072, filed Oct. 17, 2007, which claims the benefit under 35 USC 119(e) to U.S. Provisional Applications Nos. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference in its entirety.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference. The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to a method of injecting DNA are contemplated herein. The above-patents are incorporated by reference in their entirety.

Methods of preparing the nucleic acid sequences are disclosed. In some embodiments, plasmid sequences with one or more multiple cloning sites may be purchased from commercially available vendors and the expressible nucleic acid sequences disclosed herein may be ligated into the plasmids after a digestion with a known restriction enzyme needed to cute the plasmid DNA. In some embodiments, the nucleic acid molecule comprises at least one expressible nucleic acid sequence encoding a first, second and third monomeric HIV-1 ENV polypeptide or variant thereof. In some embodiments, at least one of the first, second or third monomeric HIV-1 ENV polypeptides comprises one or a plurality of mouse codons. In another alternative embodiment, membrane-based purification methods disclosed herein offer reduced cost, high binding capacity, and high flow rates, resulting in a superior purification process. The purification process is further demonstrated to produce plasmid products substantially free of genomic DNA, RNA, protein, and endotoxin.

In some embodiments, all of the described aspects of the current disclosure are advantageously combined to provide an integrated process for preparing substantially purified cellular components of interest from cells in bioreactors. Again, the cells are most preferably plasmid-containing cells, and the cellular components of interest are most preferably plasmids. The substantially purified plasmids are suitable for various uses, including, but not limited to, gene therapy, plasmid-mediated therapy, as DNA vaccines for human, veterinary, or agricultural use, or for any other application that requires large quantities of purified plasmid. In this aspect, all of the advantages described for individual aspects of the present invention accrue to the complete, integrated process, providing a highly advantageous method that is rapid, scalable, and inexpensive. Enzymes and other animal-derived or biologically sourced products are avoided, as are carcinogenic, mutagenic, or otherwise toxic substances. Potentially flammable, explosive, or toxic organic solvents are similarly avoided.

One aspect of the present disclosure is an apparatus for isolating plasmid DNA from a suspension of cells having both plasmid DNA and genomic DNA. An embodiment of the apparatus comprises a first tank and second tank in fluid communication with a mixer. The first tank is used for holding the suspension cells and the second tank is used for holding a lysis solution. The suspension of cells from the first tank and the lysis solution from the second tank are both allowed to flow into the mixer forming a lysate mixture or lysate fluid. The mixer comprises a high shear, low residence-time mixing device with a residence time of equal to or less than about 1 second. In a preferred embodiment, the mixing device comprises a flow through, rotor/stator mixer or emulsifier having linear flow rates from about 0.1 L/min to about 20 L/min. The lysate-mixture flows from the mixer into a holding coil for a period of time sufficient to lyse the cells and forming a cell lysate suspension, wherein the lysate-mixture has resident time in the holding coil in a range of about 2-8 minutes with a continuous linear flow rate. The cell lysate suspension is then allowed to flow into a bubble-mixer chamber for precipitation of cellular components from the plasmid DNA. In the bubble mixer chamber, the cell lysate suspension and a precipitation solution or a neutralization solution from a third tank are mixed together using gas bubbles, which forms a mixed gas suspension comprising a precipitate and an unclarified lysate or plasmid containing fluid. The precipitate of the mixed gas suspension is less dense than the plasmid containing fluid, which facilitates the separation of the precipitate from the plasmid containing fluid. The precipitate is removed from the mixed gas suspension to give a clarified lysate having the plasmid DNA, and the precipitate having cellular debris and genomic DNA.

In some embodiments, the bubble mixer-chamber comprises a closed vertical column with a top, a bottom, a first, and a second side with a vent proximal to the top of the column. A first inlet port of the bubble mixer-chamber is on the first side proximal to the bottom of the column and in fluid communication with the holding coil. A second inlet port of the bubble mixer-chamber is proximal to the bottom on a second side opposite of the first inlet port and in fluid communication with a third tank, wherein the third tank is used for holding a precipitation or a neutralization solution. A third inlet port of the bubble mixer-chamber is proximal to the bottom of the column and about in the middle of the first and second inlets and is in fluid communication with a gas source the third inlet entering the bubble-mixer-chamber. A preferred embodiment utilizes a sintered sparger inside the closed vertical column of the third inlet port. The outlet port exiting the bubble mixing chamber is proximal to the top of the closed vertical column. The outlet port is in fluid communication with a fourth tank, wherein the mixed gas suspension containing the plasmid DNA is allowed to flow from the bubble-mixer-chamber into the fourth tank. The fourth tank is used for separating the precipitate of the mixed gas suspension having a plasmid containing fluid, and can also include an impeller mixer sufficient to provide uniform mixing of fluid without disturbing the precipitate. A fifth tank is used for a holding the clarified lysate or clarified plasmid containing fluid. The clarified lysate is then filtered at least once. A first filter has a particle size limit of about 5-10 m and the second filter has a cut of about 0.2 m. Although gravity, pressure, vacuum, or a mixture thereof can be used for transporting: suspension of cells; lysis solutions; precipitation solutions; neutralization solutions; or mixed gas suspensions from any of the tanks to mixers, holding coils or different tanks, pumps are utilized in a preferred embodiments. In a more preferred embodiment, at least one pump having a linear flow rate from about 0.1 to about 1 ft/second is used.

In another specific embodiment, a Y-connector having a having a first bifurcated branch, a second bifurcated branch and an exit branch is used to contact the cell suspension and the lysis solutions before they enter the high shear, low residence-time mixing device. The first tank holding the cell suspension is in fluid communication with the first bifurcated branch of the Y-connector through the first pump and the second tank holding the lysis solution is in fluid communication with the second bifurcated branch of the Y-connector through the second pump. The high shear, low residence-time mixing device is in fluid communication with an exit branch of the Y-connector, wherein the first and second pumps provide a linear flow rate of about 0.1 to about 2 ft/second for a contacted fluid exiting the Y-connector.

Another specific aspect of the present invention is a method of substantially separating plasmid DNA and genomic DNA from a bacterial cell lysate. The method comprises: delivering a cell lysate into a chamber; delivering a precipitation fluid or a neutralization fluid into the chamber; mixing the cell lysate and the precipitation fluid or a neutralization fluid in the chamber with gas bubbles forming a gas mixed suspension, wherein the gas mixed suspension comprises the plasmid DNA in a fluid portion (i.e. an unclarified lysate) and the genomic DNA is in a precipitate that is less dense than the fluid portion; floating the precipitate on top of the fluid portion; removing the fluid portion from the precipitate forming a clarified lysate, whereby the plasmid DNA in the clarified lysate is substantially separated from genomic DNA in the precipitate. In preferred embodiments: the chamber is the bubble mixing chamber as described above; the lysing solution comprises an alkali, an acid, a detergent, an organic solvent, an enzyme, a chaotrope, or a denaturant; the precipitation fluid or the neutralization fluid comprises potassium acetate, ammonium acetate, or a mixture thereof; and the gas bubbles comprise compressed air or an inert gas. Additionally, the decanted-fluid portion containing the plasmid DNA is preferably further purified with one or more purification steps selected from a group consisting of ion exchange, hydrophobic interaction, size exclusion, reverse phase purification, endotoxin depletion, affinity purification, adsorption to silica, glass, or polymeric materials, expanded bed chromatography, mixed mode chromatography, displacement chromatography, hydroxyapatite purification, selective precipitation, aqueous two-phase purification, DNA condensation, thiophilic purification, ion-pair purification, metal chelate purification, filtration through nitrocellulose, or ultrafiltration.

In some embodiments, a method for isolating a plasmid DNA from cells comprising: mixing a suspension of cells having the plasmid DNA and genomic DNA with a lysis solution in a high-shear-low-residence-time-mixing-device for a first period of time forming a cell lysate fluid; incubating the cell lysate fluid for a second period of time in a holding coil forming a cell lysate suspension; delivering the cell lysate suspension into a chamber; delivering a precipitation/neutralization fluid into the chamber; mixing the cell lysate suspension and the a precipitation/neutralization fluid in the chamber with gas bubbles forming a gas mixed suspension, wherein the gas mixed suspension comprises an unclarified lysate containing the plasmid DNA and a precipitate containing the genomic DNA, wherein the precipitate is less dense than the unclarified lysate; floating the precipitate on top of the unclarified lysate; removing the precipitate from the unclarified lysate forming a clarified lysate, whereby the plasmid DNA is substantially separated from genomic DNA; precipitating the plasmid DNA from the clarified lysate forming a precipitated plasmid DNA; and resuspending the precipitated plasmid DNA in an aqueous solution.

The disclosure also relates to a method of producing a polypeptide of interest in a mammalian cell, the method comprising contacting the cell with a composition comprising a nanoparticle or the nucleic acid sequences that are RNA in the attached document. In some embodiments, the therapeutic and/or prophylactic agent is an mRNA, and wherein the mRNA encodes the polypeptide of interest, whereby the mRNA is capable of being translated in the cell to produce the polypeptide of interest. Compositions comprising RNA nucleic acid sequences of the disclosure can be delivered via lipid-containing nanoparticles and/or modification of the RNA nucleic acid sequence encoding the one or more viral polypeptides.

In some embodiments, the composition includes at least one RNA polynucleotide having an open reading frame encoding at least one HIV antigenic polypeptide having at least one modification, at least one 5′ terminal cap, and is formulated within a lipid nanoparticle.

In some embodiments, a 5′ terminal cap is 7mG(5′)ppp(5′)NlmpNp. In some embodiments, at least one chemical modification is selected from the group consisting of pseudouridine, N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine.

In some embodiments, a lipid nanoparticle comprises a cationic lipid, a PEG-modified lipid, a sterol, and a non-cationic lipid. In some embodiments, a cationic lipid is an ionizable cationic lipid and the non-cationic lipid is a neutral lipid, and the sterol is a cholesterol. In some embodiments, a cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), (12Z,15Z)—N,N-dimethyl-2-nonylhenicosa-12,15-dien-1-amine (L608), and N,N-dimethyl-1-[(1S,2R)-2-octylcyclopropyl]heptadecan-8-amine (L530).

In some embodiments, HIV RNA (e.g. mRNA) vaccines are formulated in a lipid nanoparticle. In some embodiments, HIV RNA (e.g. mRNA) vaccines are formulated in a lipid-polycation complex, referred to as a cationic lipid nanoparticle. The formation of the lipid nanoparticle may be accomplished by methods known in the art and/or as described in U.S. Publication No. 20120178702, herein incorporated by reference in its entirety. As a non-limiting example, the polycation may include a cationic peptide or a polypeptide such as, but not limited to, polylysine, polyorithine and/or polyarginine and the cationic peptides described in International Publication No. WO2012013326 or U.S. Publication No. US20130142818; each of which is herein incorporated by reference in its entirety. In some embodiments, HIV RNA (e.g. mRNA) vaccines are formulated in a lipid nanoparticle that includes a non-cationic lipid such as, but not limited to, cholesterol or dioleoyl phosphatidylethanolamine (DOPE).

A lipid nanoparticle formulation may be influenced by, but not limited to, the selection of the cationic lipid component, the degree of cationic lipid saturation, the nature of the PEGylation, ratio of all components, and biophysical parameters such as size. In one example by Semple et al. (Nature Biotech. 2010 28:172-176; herein incorporated by reference in its entirety), the lipid nanoparticle formulation is composed of 57.1% cationic lipid, 7.1% dipalmitoylphosphatidylcholine, 34.3% cholesterol, and 1.4% PEG-c-DMA. As another example, changing the composition of the cationic lipid was shown to more effectively deliver siRNA to various antigen presenting cells (Basha et al. Mol Ther. 2011 19:2186-2200; herein incorporated by reference in its entirety).

In some embodiments, lipid nanoparticle formulations may comprise 35% to 45% cationic lipid, 40% to 50% cationic lipid, 50% to 60% cationic lipid and/or 55% to 65% cationic lipid. In some embodiments, the ratio of lipid to RNA (e.g., mRNA) in lipid nanoparticles may be 5:1 to 20:1, 10:1 to 25:1, 15:1 to 30:1, and/or at least 30:1.

In some embodiments, the ratio of PEG in the lipid nanoparticle formulations may be increased or decreased and/or the carbon chain length of the PEG lipid may be modified from C14 to C18 to alter the pharmacokinetics and/or biodistribution of the lipid nanoparticle formulations. As a non-limiting example, lipid nanoparticle formulations may contain 0.5% to 3.0%, 1.0% to 3.5%, 1.5% to 4.0%, 2.0% to 4.5%, 2.5% to 5.0%, and/or 3.0% to 6.0% of the lipid molar ratio of PEG-c-DOMG (R-3-[(co-methoxy-poly(ethyleneglycol)2000) carbamoyl)]-1,2-dimyristyloxypropyl-3-amine) (also referred to herein as PEG-DOMG) as compared to the cationic lipid, DSPC, and cholesterol. In some embodiments, the PEG-c-DOMG may be replaced with a PEG lipid such as, but not limited to, PEG-DSG (1,2-Distearoyl-sn-glycerol, methoxypolyethylene glycol), PEG-DMG (1,2-Dimyristoyl-sn-glycerol) and/or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA. C12-200, and DLin-KC2-DMA.

In some embodiments, a HIV RNA (e.g., mRNA) vaccine formulation is a nanoparticle that comprises at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG, (12Z,15Z)—N,N-dimethyl-2-nonylhenicosa-12,15-dien-1-amine (L608), N,N-dimethyl-1-[(1S,2R)-2-octylcyclopropyl]heptadecan-8-amine (L530), PEGylated lipids, and amino alcohol lipids.

In some embodiments, a lipid nanoparticle formulation includes 25% to 75% on a molar basis of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), e.g., 35% to 65%, 45% to 65%, 60%, 57.5%, 50% or 40% on a molar basis.

In some embodiments, a lipid nanoparticle formulation includes 0.5% to 15% on a molar basis of the neutral lipid, e.g., 3% to 12%, 5% to 10% or 15%, 10%, or 7.5% on a molar basis. Examples of neutral lipids include, without limitation, DSPC, POPC, DPPC, DOPE, and SM. In some embodiments, the formulation includes 5% to 50% on a molar basis of the sterol (e.g., 15% to 45%, 20% to 40%, 40%, 38.5%, 35%, or 31% on a molar basis. A non-limiting example of a sterol is cholesterol. In some embodiments, a lipid nanoparticle formulation includes 0.5% to 20% on a molar basis of the PEG or PEG-modified lipid (e.g., 0.5% to 10%, 0.5% to 5%, 1.5%, 0.5%, 1.5%, 3.5%, or 5% on a molar basis. In some embodiments, a PEG or PEG modified lipid comprises a PEG molecule of an average molecular weight of 2,000 Da. In some embodiments, a PEG or PEG modified lipid comprises a PEG molecule of an average molecular weight of less than 2,000, for example around 1,500 Da, around 1,000 Da, or around 500 Da. Non-limiting examples of PEG-modified lipids include PEG-distearoyl glycerol (PEG-DMG) (also referred herein as PEG-C14 or C14-PEG), and PEG-cDMA (further discussed in Reyes et al. J. Controlled Release, 107, 276-287 (2005) the content of which is herein incorporated by reference in its entirety).

In some embodiments, lipid nanoparticle formulations include 25-75% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 0.5-15% of the neutral lipid, 5-50% of the sterol, and 0.5-20% of the PEG or PEG-modified lipid on a molar basis.

In some embodiments, lipid nanoparticle formulations include 35-65% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 3-12% of the neutral lipid, 15-45% of the sterol, and 0.5-10% of the PEG or PEG-modified lipid on a molar basis.

In some embodiments, lipid nanoparticle formulations include 45-65% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 5-10% of the neutral lipid, 25-40% of the sterol, and 0.5-10% of the PEG or PEG-modified lipid on a molar basis.

In some embodiments, lipid nanoparticle formulations include 60% of a cationic lipid selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319), 7.5% of the neutral lipid, 31% of the sterol, and 1.5% of the PEG or PEG-modified lipid on a molar basis.

Some embodiments of the present disclosure provide a HIV vaccine that includes at least one ribonucleic acid (RNA) polynucleotide having an open reading frame encoding at least one HIV antigenic polypeptide, wherein at least about 80% of the uracil in the open reading frame have a chemical modification, optionally wherein the HIV vaccine is formulated in a lipid nanoparticle. In some embodiments, the RNA vaccine pharmaceutical compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12)1708-1713); herein incorporated by reference in its entirety) and hyaluronan-coated liposomes (Quiet Therapeutics, Israel). In some embodiments, the RNA vaccines may be formulated in a lyophilized gel-phase liposomal composition as described in U.S. Publication No. US2012060293, herein incorporated by reference in its entirety.

The nanoparticle formulations may comprise a phosphate conjugate. The phosphate conjugate may increase in vivo circulation times and/or increase the targeted delivery of the nanoparticle. Phosphate conjugates for use with the present invention may be made by the methods described in International Publication No. WO2013033438 or U.S. Publication No. US20130196948, the content of each of which is herein incorporated by reference in its entirety. As a non-limiting example, the phosphate conjugates may include a compound of any one of the formulas described in International Publication No. WO2013033438, herein incorporated by reference in its entirety. In particular, the present invention relates to a pharmaceutical composition comprising nanoparticles which comprise RNA encoding at least one antigen, wherein:

(i) the number of positive charges in the nanoparticles does not exceed the number of negative charges in the nanoparticles and/or

(ii) the nanoparticles have a neutral or net negative charge and/or

(iii) the charge ratio of positive charges to negative charges in the nanoparticles is 1.4:1 or less and/or

(iv) the zeta potential of the nanoparticles is 0 or less.

In some embodiments, the nanoparticles described herein are colloidally stable for at least 2 hours in the sense that no aggregation, precipitation or increase of size and polydispersity index by more than 30% as measured by dynamic light scattering takes place. In some embodiments, the charge ratio of positive charges to negative charges in the nanoparticles is between 1.4:1 and 1:8, preferably between 1.2:1 and 1:4, e.g. between 1:1 and 1:3 such as between 1:1.2 and 1:2, 1:1.2 and 1:1.8, 1:1.3 and 1:1.7, in particular between 1:1.4 and 1:1.6, such as about 1:1.5. In some embodiments, the zeta potential of the nanoparticles is −5 or less, −10 or less, −15 or less, −20 or less or −25 or less. In various embodiments, the zeta potential of the nanoparticles is −35 or higher, −30 or higher or −25 or higher. In some embodiments, the nanoparticles have a zeta potential from 0 mV to −50 mV, preferably 0 mV to −40 mV or −10 mV to −30 mV.

In some embodiments pharmaceutical compositions of the disclosure comprise a nanoparticle or a liposome that encapsulates a DNA, RNA or DNA/RNA hybrid comprising at least one expressible nucleic acid sequence. Liposomes are microscopic lipidic vesicles often having one or more bilayers of a vesicle-forming lipid, such as a phospholipid, and are capable of encapsulating a drug. Different types of liposomes may be employed in the context of the present invention, including, without being limited thereto, multilamellar vesicles (MLV), small unilamellar vesicles (SUV), large unilamellar vesicles (LUV), sterically stabilized liposomes (SSL), multivesicular vesicles (MV), and large multivesicular vesicles (LMV) as well as other bilayered forms known in the art. The size and lamellarity of the liposome will depend on the manner of preparation and the selection of the type of vesicles to be used will depend on the preferred mode of administration. There are several other forms of supramolecular organization in which lipids may be present in an aqueous medium, comprising lamellar phases, hexagonal and inverse hexagonal phases, cubic phases, micelles, reverse micelles composed of monolayers. These phases may also be obtained in the combination with DNA or RNA, and the interaction with RNA and DNA may substantially affect the phase state. The described phases may be present in the nanoparticulate RNA formulations of the present invention.

For formation of RNA lipoplexes from RNA and liposomes, any suitable method of forming liposomes can be used so long as it provides the envisaged RNA lipoplexes. Liposomes may be formed using standard methods such as the reverse evaporation method (REV), the ethanol injection method, the dehydration-rehydration method (DRV), sonication or other suitable methods.

After liposome formation, the liposomes can be sized to obtain a population of liposomes having a substantially homogeneous size range.

Bilayer-forming lipids have typically two hydrocarbon chains, particularly acyl chains, and a head group, either polar or nonpolar. Bilayer-forming lipids are either composed of naturally-occurring lipids or of synthetic origin, including the phospholipids, such as phosphatidylcholine, phosphatidylethanolamine, phosphatide acid, phosphatidylinositol, and sphingomyelin, where the two hydrocarbon chains are typically between about 14-22 carbon atoms in length, and have varying degrees of unsaturation. Other suitable lipids for use in the composition of the present invention include glycolipids and sterols such as cholesterol and its various analogs which can also be used in the liposomes.

Cationic lipids typically have a lipophilic moiety, such as a sterol, an acyl or diacyl chain, and have an overall net positive charge. The head group of the lipid typically carries the positive charge. The cationic lipid preferably has a positive charge of 1 to 10 valences, more preferably a positive charge of 1 to 3 valences, and more preferably a positive charge of 1 valence. Examples of cationic lipids include, but are not limited to 1,2-di-O-octadecenyl-3-trimethylammonium propane (DOTMA); dimethyldioctadecylammonium (DDAB); 1,2-dioleoyl-3-trimethylammonium-propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-dimyristoyloxypropyl-1,3-dimethylhydroxyethyl ammonium (DMRIE), and 2,3-dioleoyloxy-N-[2(spermine carboxamide)ethyl]-N,N-dimethyl-1-propanamium trifluoroacetate (DOSPA). Preferred are DOTMA, DOTAP, DODAC, and DOSPA. Most preferred is DOTMA.

In addition, the nanoparticles described herein preferably further include a neutral lipid in view of structural stability and the like. The neutral lipid can be appropriately selected in view of the delivery efficiency of the RNA-lipid complex. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, sterol, and cerebroside. Preferred is DOPE and/or DOPC. Most preferred is DOPE. In the case where a cationic liposome includes both a cationic lipid and a neutral lipid, the molar ratio of the cationic lipid to the neutral lipid can be appropriately determined in view of stability of the liposome and the like.

According to one embodiment, the nanoparticles described herein may comprise phospholipids. The phospholipids may be a glycerophospholipid. Examples of glycerophospholipid include, without being limited thereto, three types of lipids: (i) zwitterionic phospholipids, which include, for example, phosphatidylcholine (PC), egg yolk phosphatidylcholine, soybean-derived PC in natural, partially hydrogenated or fully hydrogenated form, dimyristoyl phosphatidylcholine (DMPC) sphingomyelin (SM); (ii) negatively charged phospholipids: which include, for example, phosphatidylserine (PS), phosphatidylinositol (PI), phosphatidic acid (PA), phosphatidylglycerol (PG) dipalmipoyl PG, dimyristoyl phosphatidylglycerol (DMPG); synthetic derivatives in which the conjugate renders a zwitterionic phospholipid negatively charged such is the case of methoxy-polyethylene,glycol-distearoyl phosphatidylethanolamine (mPEG-DSPE); and (iii) cationic phospholipids, which include, for example, phosphatidylcholine or sphingomyelin of which the phosphomonoester was O-methylated to form the cationic lipids.

Association of RNA to the lipid carrier can occur, for example, by the RNA filling interstitial spaces of the carrier, such that the carrier physically entraps the RNA, or by covalent, ionic, or hydrogen bonding, or by means of adsorption by non-specific bonds. Whatever the mode of association, the RNA must retain its therapeutic, i.e. antigen-encoding, properties.

In some embodiments, the nanoparticles comprise at least one lipid. In some embodiments, the nanoparticles comprise at least one cationic lipid. The cationic lipid can be monocationic or polycationic. Any cationic amphiphilic molecule, e.g., a molecule which comprises at least one hydrophilic and lipophilic moiety is a cationic lipid within the meaning of the present invention. In some embodiments, the positive charges are contributed by the at least one cationic lipid and the negative charges are contributed by the RNA. In some embodiments, the nanoparticles comprises at least one helper lipid. The helper lipid may be a neutral or an anionic lipid. The helper lipid may be a natural lipid, such as a phospholipid or an analogue of a natural lipid, or a fully synthetic lipid, or lipid-like molecule, with no similarities with natural lipids. In some embodiments, the cationic lipid and/or the helper lipid is a bilayer forming lipid.

In some embodiments, the at least one cationic lipid comprises 1,2-di-O-octadecenyl-3-trimethylammonium propane (DOTMA) or analogs or derivatives thereof and/or 1,2-dioleoyl-3-trimethylammonium-propane (DOTAP) or analogs or derivatives thereof. In some embodiments, the at least one helper lipid comprises 1,2-di-(9Z-octadecenoyl)-sn-glycero-3-phosphoethanolamine (DOPE) or analogs or derivatives thereof, cholesterol (Chol) or analogs or derivatives thereof and/or 1,2-dioleoyl-sn-glycero-3-phosphocholine (DOPC) or analogs or derivatives thereof. In some embodiments, the molar ratio of the at least one cationic lipid to the at least one helper lipid is from 10:0 to 3:7, preferably 9:1 to 3:7, 4:1 to 1:2, 4:1 to 2:3, 7:3 to 1:1, or 2:1 to 1:1, preferably about 1:1. In some embodiments, in this ratio, the molar amount of the cationic lipid results from the molar amount of the cationic lipid multiplied by the number of positive charges in the cationic lipid. In various embodiments, the lipids are not functionalized such as functionalized by mannose, histidine and/or imidazole, the nanoparticles do not comprise a targeting ligand such as mannose functionalized lipids and/or the nanoparticles do not comprise one or more of the following: pH dependent compounds, cationic polymers such as polymers containing histidine and/or polylysine, wherein the polymers may optionally be PEGylated and/or histidylated, or divalent ions such as Ca 2+.

In various embodiments, the RNA nanoparticles may comprise peptides, preferentially with a molecular weight of up to 2500 Da.

In the nanoparticles described herein the lipid may form a complex with and/or may encapsulate the RNA. In some embodiments, the nanoparticles comprise a lipoplex or liposome. In some embodiments, the lipid is comprised in a vesicle encapsulating said RNA. The vesicle may be a multilamellar vesicle, an unilamellar vesicle, or a mixture thereof. The vesicle may be a liposome. In some embodiments, the nanoparticles are lipoplexes comprising DOTMA and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2.

In some embodiments, the nanoparticles are lipoplexes comprising DOTMA and Cholesterol in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In some embodiments, the nanoparticles are lipoplexes comprising DOTAP and DOPE in a molar ratio of 10:0 to 1:9, preferably 8:2 to 3:7, and more preferably of 7:3 to 5:5 and wherein the charge ratio of positive charges in DOTMA to negative charges in the RNA is 1.8:2 to 0.8:2, more preferably 1.6:2 to 1:2, even more preferably 1.4:2 to 1.1:2 and even more preferably about 1.2:2. In some embodiments, the nanoparticles are lipoplexes comprising DOTMA and DOPE in a molar ratio of 2:1 to 1:2, preferably 2:1 to 1:1, and wherein the charge ratio of positive charges in DOTMA to negative charges in the RNA is 1.4:1 or less. In some embodiments, the nanoparticles are lipoplexes comprising DOTMA and cholesterol in a molar ratio of 2:1 to 1:2, preferably 2:1 to 1:1, and wherein the charge ratio of positive charges in DOTMA to negative charges in the RNA is 1.4:1 or less. In some embodiments, the nanoparticles are lipoplexes comprising DOTAP and DOPE in a molar ratio of 2:1 to 1:2, preferably 2:1 to 1:1, and wherein the charge ratio of positive charges in DOTAP to negative charges in the RNA is 1.4:1 or less. In some embodiments, the nanoparticles have an average diameter in the range of from about 50 nm to about 1000 nm, preferably from about 50 nm to about 400 nm, preferably about 100 nm to about 300 nm such as about 150 nm to about 200 nm. In some embodiments, the nanoparticles have a diameter in the range of about 200 to about 700 nm, about 200 to about 600 nm, preferably about 250 to about 550 nm, in particular about 300 to about 500 nm or about 200 to about 400 nm.

In some embodiments, the polydispersity index of the nanoparticles described herein as measured by dynamic light scattering is 0.5 or less, preferably 0.4 or less or even more preferably 0.3 or less. In some embodiments, the nanoparticles described herein are obtainable by one or more of the following: (i) incubation of liposomes in an aqueous phase with the RNA in an aqueous phase, (ii) incubation of the lipid dissolved in an organic, water miscible solvent, such as ethanol, with the RNA in aqueous solution, (iii) reverse phase evaporation technique, (iv) freezing and thawing of the product, (v) dehydration and rehydration of the product, (vi) lyophilization and rehydration of the of the product, or (vii) spray drying and rehydration of the product.

The nanoparticle formulation may comprise a polymer conjugate. The polymer conjugate may be a water-soluble conjugate. The polymer conjugate may have a structure as described in U.S. Publication No. 20130059360, the content of which is herein incorporated by reference in its entirety. In some aspects, polymer conjugates with the polynucleotides of the present invention may be made using the methods and/or segmented polymeric reagents described in U.S. Publication No. 20130072709, herein incorporated by reference in its entirety. In other aspects, the polymer conjugate may have pendant side groups comprising ring moieties such as, but not limited to, the polymer conjugates described in U.S. Publication No. US20130196948, the contents of which is herein incorporated by reference in its entirety.

The nanoparticle formulations may comprise a conjugate to enhance the delivery of nanoparticles of the present invention in a subject. Further, the conjugate may inhibit phagocytic clearance of the nanoparticles in a subject. In some aspects, the conjugate may be a “self” peptide designed from the human membrane protein CD47 (e.g., the “self” particles described by Rodriguez et al. (Science 2013, 339, 971-975), herein incorporated by reference in its entirety). As shown by Rodriguez et al., the self peptides delayed macrophage-mediated clearance of nanoparticles which enhanced delivery of the nanoparticles. In other aspects, the conjugate may be the membrane protein CD47 (e.g., see Rodriguez et al. Science 2013, 339, 971-975, herein incorporated by reference in its entirety). Rodriguez et al. showed that, similarly to “self” peptides, CD47 can increase the circulating particle ratio in a subject as compared to scrambled peptides and PEG coated nanoparticles.

In some embodiments, 100% of the uracil in the open reading frame have a chemical modification. In some embodiments, a chemical modification is in the 5-position of the uracil. In some embodiments, a chemical modification is a N1-methyl pseudouridine. In some embodiments, 100% of the uracil in the open reading frame have a N1-methyl pseudouridine in the 5-position of the uracil.

In some embodiments, efficacy of RNA vaccines RNA (e.g., mRNA) can be significantly enhanced when combined with a flagellin adjuvant, in particular, when one or more antigen-encoding mRNAs is combined with an mRNA encoding flagellin.

RNA (e.g., mRNA) vaccines combined with the flagellin adjuvant (e.g., mRNA-encoded flagellin adjuvant) have superior properties in that they may produce much larger antibody titers and produce responses earlier than commercially available vaccine formulations. While not wishing to be bound by theory, it is believed that the RNA vaccines, for example, as mRNA polynucleotides, are better designed to produce the appropriate protein conformation upon translation, for both the antigen and the adjuvant, as the RNA (e.g., mRNA) vaccines co-opt natural cellular machinery. Unlike traditional vaccines, which are manufactured ex vivo and may trigger unwanted cellular responses, RNA (e.g., mRNA) vaccines are presented to the cellular system in a more native fashion.

Some embodiments of the present disclosure provide RNA (e.g., mRNA) vaccines that include at least one RNA (e.g., mRNA) polynucleotide having an open reading frame encoding at least one antigenic polypeptide or an immunogenic fragment thereof (e.g., an immunogenic fragment capable of inducing an immune response to the antigenic polypeptide) and at least one RNA (e.g., mRNA polynucleotide) having an open reading frame encoding a flagellin adjuvant.

In some embodiments, at least one flagellin polypeptide (e.g., encoded flagellin polypeptide) is a flagellin protein. In some embodiments, at least one flagellin polypeptide (e.g., encoded flagellin polypeptide) is an immunogenic flagellin fragment. In some embodiments, at least one flagellin polypeptide and at least one antigenic polypeptide are encoded by a single RNA (e.g., mRNA) polynucleotide. In other embodiments, at least one flagellin polypeptide and at least one antigenic polypeptide are each encoded by a different RNA polynucleotide.

Some embodiments of the present disclosure provide methods of inducing an antigen specific immune response in a subject, comprising administering to the subject a HIV vaccine in an amount effective to produce an antigen specific immune response.

In some aspects, vaccines of the invention (e.g., LNP-encapsulated mRNA vaccines) produce prophylactically- and/or therapeutically-efficacious levels, concentrations and/or titers of antigen-specific antibodies in the blood or serum of a vaccinated subject. As defined herein, the term antibody titer refers to the amount of antigen-specific antibody produces in s subject, e.g., a human subject. In exemplary embodiments, antibody titer is expressed as the inverse of the greatest dilution (in a serial dilution) that still gives a positive result. In exemplary embodiments, antibody titer is determined or measured by enzyme-linked immunosorbent assay (ELISA). In exemplary embodiments, antibody titer is determined or measured by neutralization assay, e.g., by microneutralization assay. In certain aspects, antibody titer measurement is expressed as a ratio, such as 1:40, 1:100, etc.

In exemplary embodiments of the invention, an efficacious vaccine produces an antibody titer of greater than 1:40, greater that 1:100, greater than 1:400, greater than 1:1000, greater than 1:2000, greater than 1:3000, greater than 1:4000, greater than 1:500, greater than 1:6000, greater than 1:7500, greater than 1:10000. In exemplary embodiments, the antibody titer is produced or reached by 10 days following vaccination, by 20 days following vaccination, by 30 days following vaccination, by 40 days following vaccination, or by 50 or more days following vaccination. In exemplary embodiments, the titer is produced or reached following a single dose of vaccine administered to the subject. In other embodiments, the titer is produced or reached following multiple doses, e.g., following a first and a second dose (e.g., a booster dose.)

In exemplary aspects of the invention, antigen-specific antibodies are measured in units of g/ml or are measured in units of IU/L (International Units per liter) or mIU/ml (milli International Units per ml). In exemplary embodiments of the invention, an efficacious vaccine produces >0.5 μg/ml, >0.1 μg/ml, >0.2 μg/ml, >0.35 μg/ml, >0.5 μg/ml, >1 μg/ml, >2 μg/ml, >5 μg/ml or >10 μg/ml. In exemplary embodiments of the invention, an efficacious vaccine produces >10 mIU/ml, >20 mIU/ml, >50 mIU/ml, >100 mIU/ml, >200 mIU/ml, >500 mIU/ml or >1000 mIU/ml. In exemplary embodiments, the antibody level or concentration is produced or reached by 10 days following vaccination, by 20 days following vaccination, by 30 days following vaccination, by 40 days following vaccination, or by 50 or more days following vaccination. In exemplary embodiments, the level or concentration is produced or reached following a single dose of vaccine administered to the subject. In other embodiments, the level or concentration is produced or reached following multiple doses, e.g., following a first and a second dose (e.g., a booster dose.) In exemplary embodiments, antibody level or concentration is determined or measured by enzyme-linked immunosorbent assay (ELISA). In exemplary embodiments, antibody level or concentration is determined or measured by neutralization assay, e.g., by microneutralization assay.

In some embodiments, the HIV vaccine includes at least one RNA polynucleotide having an open reading frame encoding at least one HIV antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap]; G(5′)ppp(5′)A; G(5′)ppp(5′)G; m7G(5′)ppp(5′)A; m7G(5′)ppp(5′)G (New England BioLabs, Ipswich, Mass.). 5′-capping of modified RNA may be completed post-transcriptionally using a Vaccinia Virus Capping Enzyme to generate the “Cap 0” structure: m7G(5′)ppp(5′)G (New England BioLabs, Ipswich, Mass.). Cap 1 structure may be generated using both Vaccinia Virus Capping Enzyme and a 2′-O methyl-transferase to generate m7G(5′)ppp(5′)G-2′-O-methyl. Cap 2 structure may be generated from the Cap 1 structure followed by the 2′-O-methylation of the 5′-antepenultimate nucleotide using a 2′-0 methyl-transferase. Cap 3 structure may be generated from the Cap 2 structure followed by the 2′-O-methylation of the 5′-preantepenultimate nucleotide using a 2′-0 methyl-transferase. Enzymes are preferably derived from a recombinant source.

When transfected into mammalian cells, the modified mRNAs have a stability of from about 12 to about 18 hours or more than about 18 hours, e.g., 24, 36, 48, 60, 72, or greater than about 72 hours.

In some embodiments, a codon optimized RNA may, for instance, be one in which the levels of G/C are enhanced. The G/C-content of nucleic acid molecules may influence the stability of the RNA. RNA having an increased amount of guanine (G) and/or cytosine (C) residues may be functionally more stable than nucleic acids containing a large amount of adenine (A) and thymine (T) or uracil (U) nucleotides. WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

Modifications of polynucleotides (e.g., RNA polynucleotides, such as mRNA polynucleotides), including but not limited to chemical modification, that are useful in the compositions, vaccines, methods and synthetic processes of the present disclosure include, but are not limited to the following: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; a-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-lodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcvtidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; c-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methyiwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; a-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-ethyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methyluridine,), 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uracil; NT-ethyl-pseudo-uracil; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; a-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 1-Ethyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′azido, 2′fluoro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (+) 1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl} pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cvcloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2(2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}] propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluoro-cytidine; 2′ methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostvrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-aminopyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (p), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (mly), 1-ethyl-pseudouridine (elyi), 5-methoxy-uridine (mo5U), 5-methyl-cytidine (m5C), a-thio-guanosine, a-thio-adenosine, 5-cyano uridine, 4′-thio uridine 7-deaza-adenine, 1-methyl-adenosine (mlA), 2-methyl-adenine (m2A), N6-methyl-adenosine (m6A), and 2,6-Diaminopurine, (I), 1-methyl-inosine (mlI), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (mlG), 8-oxo-guanosine, 7-methyl-8-oxo-guanosine, 2,8-dimethyladenosine, 2-geranylthiouridine, 2-lysidine, 2-selenouridine, 3-(3-amino-3-carboxypropyl)-5,6-dihydrouridine, 3-(3-amino-3-carboxypropyl)pseudouridine, 3-methylpseudouridine, 5-(carboxyhydroxymethyl)-2′-O-methyluridine methyl ester, 5-aminomethyl-2-geranylthiouridine, 5-aminomethyl-2-selenouridine, 5-aminomethyluridine, 5-carbamoylhydroxymethyluridine, 5-carbamoylmethyl-2-thiouridine, 5-carboxymethyl-2-thiouridine, 5-carboxymethylaminomethyl-2-geranylthiouridine, 5-carboxymethylaminomethyl-2-selenouridine, 5-cyanomethyluridine, 5-hydroxycytidine, 5-methylaminomethyl-2-geranylthiouridine, 7-aminocarboxypropyl-demethylwyosine, 7-aminocarboxypropylwyosine, 7-aminocarboxypropylwyosine methyl ester, 8-methyladenosine, N4,N4-dimethylcytidine, N6-formyladenosine, N6-hydroxymethyladenosine, agmatidine, cyclic N6-threonylcarbamoyladenosine, glutamyl-queuosine, methylated undermodified hydroxywybutosine, N4,N4,2′-O-trimethylcytidine, geranylated 5-methylaminomethyl-2-thiouridine, geranylated 5-carboxymethylaminomethyl-2-thiouridine, Qbase, preQObase, preQIbase, and combinations of two or more thereof. In some embodiments, the at least one chemically modified nucleoside is selected from the group consisting of pseudouridine, 1-methyl-pseudouridine, 1-ethyl-pseudouridine, 5-methylcytosine, 5-methoxyuridine, and a combination thereof. In some embodiments, the polyribonucleotide (e.g., RNA polyribonucleotide, such as mRNA polyribonucleotide) includes a combination of at least two (e.g., 2, 3, 4 or more) of the aforementioned modified nucleobases. In some embodiments, polynucleotides (e.g., RNA polynucleotides, such as mRNA polynucleotides) include a combination of at least two (e.g., 2, 3, 4 or more) of the aforementioned modified nucleobases.

The expressible nucleic acid sequence of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the invention, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may be any one of nucleotides A, G, U, C, or any one of the combinations A+G, A+U, A+C, G+U, G+C, U+C, A+G+U, A+G+C, G+U+C, or A+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 252%, from 1T % to 50%, from about 1T % to about 60%, from 1% to 70%, from 1% to 80%, from 1% to 90%, from 10% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acid sequences may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4, or more unique structures). In some embodiments, at least 5%, at least 10%, at least 25%, at least 50%, at least 80%, at least 90%, or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4, or more unique structures).

Thus, in some embodiments, the RNA vaccines and/or RNA nucleic acid sequences comprise a 5′UTR element, an optionally codon optimized open reading frame, and a 3′UTR element, a poly(A) sequence and/or a polyadenylation signal wherein the RNA is not chemically modified.

Viral vaccines of the present disclosure comprise at least one RNA polynucleotide, such as a mRNA (e.g., modified mRNA). mRNA, for example, is transcribed in vitro from template DNA, referred to as an “in vitro transcription template.” In some embodiments, the at least one RNA polynucleotide has at least one chemical modification. The at least one chemical modification may include, but is expressly not limited to, any modification described herein.

In vitro transcription of RNA is known in the art and is described in WO/2014/152027, which is incorporated by reference herein in its entirety. For example, in some embodiments, the RNA transcript is generated using a non-amplified, linearized DNA template in an in vitro transcription reaction to generate the RNA transcript. In some embodiments, the RNA transcript is capped via enzymatic capping. In some embodiments, the RNA transcript is purified via chromatographic methods, e.g., use of an oligo dT substrate. Some embodiments exclude the use of DNase. In some embodiments, the RNA transcript is synthesized from a non-amplified, linear DNA template coding for the gene of interest via an enzymatic in vitro transcription reaction utilizing a T7 phage RNA polymerase and nucleotide triphosphates of the desired chemistry. Any number of RNA polymerases or variants may be used in the method of the present invention. The polymerase may be selected from, but is not limited to, a phage RNA polymerase, e.g., a T7 RNA polymerase, a T3 RNA polymerase, a SP6 RNa polymerase, and/or mutant polymerases such as, but not limited to, polymerases able to incorporate modified nucleic acids and/or modified nucleotides, including chemically modified nucleic acids and/or nucleotides.

In some embodiments, a non-amplified, linearized plasmid DNA is utilized as the template DNA for in vitro transcription. In some embodiments, the template DNA is isolated DNA. In some embodiments, the template DNA is cDNA. In some embodiments, the cDNA is formed by reverse transcription of a RNA polynucleotide, for example, but not limited to HIV RNA, e.g. HIV mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

### E. Vaccines

Disclosed are vaccines comprising a first amino acid sequence comprising at least about 70%, 75%, 80%, 85%, 90%, 95%, 96%, 97%, 98%, 99% or 100% sequence identity to any linker sequence provided herein; and/or a second amino acid sequence comprising at least about 70%, 75%, 80%, 85%, 90%, 95%, 96%, 97%, 98%, 99% or 100% sequence identity to any one or combination of viral antigens (such as any one or combination of gp41 or gp120 nucleic acid sequences) disclosed herein. In some embodiments, the vaccines are free of a nucleic acid sequence that encodes an HIV transmembrane domain (gp41). In some cases the vaccine is a DNA or RNA vaccine that, upon administration to a subject and upon contact with a cell, encodes for a soluble retorviral trimer molecule. In some cases the vaccine is a DNA or RNA vaccine that, upon administration to a subject and upon contact with a cell, encodes for a soluble HIV ENV trimer molecule.

In some embodiments, the vaccines further comprise a linker fusing a first and a second nucleic acid sequence that encodes an amino acid sequence that is a fusion protein. For example, the linker can be an amino acid sequence comprising at least about 70%, 75%, 80%, 85%, 90%, 95%, 96%, 97%, 98%, 99% or 100% sequence identity to SEQ ID NO:8.

### F. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising any of the elements of the disclosed nucleic acid compositions. For example, disclosed are kits comprising nucleic acid sequences comprising a leader sequence, a linker sequence, a nucleic acid sequence encoding a soluble retorviral envelope polypeptide. In some embodiments, the kits can further comprise a plasmid backbone.

### EXAMPLES

### 1. Methods

i. DNA Design and Plasmid Synthesis

Amino acid sequences for BG505_MD39 based stabilized trimers were obtained from Kulp et al[8]. These sequences were then RNA and codon optimized as well as optimizing for GC content and secondary structure. Additionally, an optimized IgE leader sequence was added to the C term of the protein to provide efficient processing and secretion. All plasmid inserts were cloned into our modified pVAX1 backbone. Additional mutations were made to the BG505_MD39 base trimer to explore cleavage dependence, circular permutations, adding glycosylation to the bottom of the trimer, creating strings of trimers as well as linking the trimers to the membrane by including a transmembrane (PDGFR) domain.

Plasmids that encode the HIV Envelope BG505 WT was obtained from GenBank and produced. Point mutations were made for BG505 T332N, BG505 T332N S241N, BG505 T332N T456N. MG505, HIV backbone delta Env and MLV plasmids were obtained from NIH AIDS reagents resources. Plasmids for 11A and 12N antibodies were synthesized by Genscript and cloned into the modified pVAX1 backbone.

ii. Cell Lines, Transfection and Recombinant Antibody Purification.

HEK 293T cells and TZM-bl cells were maintained in DMEM supplemented with 10% of heat inactivated fetal bovine serum. Expi293F cells were maintained in Expi293 expression medium.

To produce recombinant HIV monoclonal antibodies for assay and controls, Expi293F cells were transfected following manufactures protocol for Expifectamin. Transfection enhancers were added 18 hours after transfection and supernatants were harvested 6 days after transfection. Protein G agarose was then used following manufactures protocol to purify out the IgG. Purity was confirmed with commassie staining of SDS-page gels and quantified using the quantification ELISA described below.

Pseudotype viruses were produced by transfecting HEK 293T cells with plasmid expressing the Env of interest with the plasmid expressing the HIV-1 backbone delta Env using GeneJammer. Forty-eight hours after transfection, cell supernatant was harvested and filtered through a 45 um filter.

iii. Production of Trimer

BG505_MD39-based trimers were expressed in FreeStyle 293F Cells and are derived from a low-passage Master Cell Bank and certified mycoplasma free. The trimer-containing supernants were obtained by centrifuging (4000×g, 25 mins) and filtering (0.2 um Nalgene Rapid-Flow Filter) the 293F cultures. Trimers were purified from supernants by lectin purification using lectin beads (7.5 ml beads/1 L culture) and lectin elution buffer (1M Methyl alpha-D-mannopyranoside). The elution was dialyzed overnight into PBS. The trimers were then purified over a size-exclusion chromatography column (GE S200 Increase) in PBS. The molecular weight and homogeneity of the trimers were confirmed by protein conjugated analysis from ASTRA with data collected from a size-exclusion chromatography-multi-angle light scattering (SEC-MALS) experiment run in PBS using a GE S6 Increase column followed by DAWN HELEOS II and Optilab T-rEX detectors. The trimers were aliquoted at 1 mg/ml and flash frozen in thin-walled PCR tubes prior to use.

iv. Immunization of Mice

All mice were housed in compliance with the NIH and Wistar's Institutional Animal Care and Use Committee guidelines. To test for immunogenicity, 6-8 week old BalbC mice were immunized with 25 ug of each plasmid followed by in vivo electroporation using the CELLECTA® 3P adaptive constant current electroporation device. Mice were immunized at either 0, 3, 6 or 0, 3, 16 and sacrificed one week after final immunization to assess vaccine induced immune responses. A subset of mice were given recombinant protein trimer formulated in RIBI adjuvant at a 25 ug dose delivered to two sites subq at weeks 0, 3, 6.

v. Immunization of Rabbits

All rabbits were housed and handled according to the standards of the Institutional Animal Care and Use Committee (IACUC) at BioTox Sciences (San Diego, Calif.). Female New Zealand white rabbits (1900 grams) were immunized using 1-2 mg plasmid of DNA intradermal at weeks 0, 4, 12, 20 with in vivo EP as described above. All rabbits received two injection sites. Blood was collected for analysis at weeks −2, 2, 6, 12, 14, 17, 20, 22, and 28.

vi. Immunization of Non-Human Primates

Ten rhesus macaques were housed at Bioqual (Rockville Md.) according to the standards of the American Association for Accreditation of Laboratory Animal Care, and all animal protocols were IACUC approved. All animals received four vaccinations all via the intradermal route. Half of the NHPs received 2 mg of pMD39_Gly_opt and the other half received 2 mg of the pMD39_TS1 delivered to two sites. The animals were vaccinated on weeks 0, 4, 12, 20. All DNA deliveries were followed by in vivo EP with the constant current CELLECTRA® device with three pulses at 0.5 A constant current, a 52 ms pulse length and is rest between pulses.

vii. Blood Collection

NHPs were bled at weeks −2, 2, 6, 12, 14, 20, 22, and 28. Blood (15 ml at each time point) was collected in EDTA tubes, and peripheral blood mononuclear cells (PBMCs) were isolated using the standard Ficoll-Hypaque procedure with Accuspin tubes (Sigma-Aldrich). An additional 10 ml was collected into clot tubes for serum collection.

viii. Mouse IFN-Gamma Enzyme-Linked Immunospot Assay (ELISpot)

Ninety-six well filter plates were pre-coated with anti-IFN-γ capture antibody.

Spleens were isolated from mice two week after final immunization. After processing the spleens to obtain a single cell suspension, 2×105 cells were added to the blocked plates. Cells were stimulated with overlapping 15mer peptide pools for WT BG505 gp160 (5 ug/ml per peptide). Media alone and concanavalin A were used as negative and positive controls respectively. After 18 hrs of stimulation, the plates were washed, and detection antibody (R4-6A2-biotin) was added for 2 hrs at RT. Plates were then washed and the Streptavidin-ALP antibody was added for 1 hour at RT. Plates were then developed using the BCIP/NBT-plus for 10 minutes. Plates were then scanned and counted using CTL-ImmunoSpot® S6 FluoroSpot plate reader.

ix. Intracellular Cytokine Staining

For intracellular cytokine staining, 2×106 splenocytes were stimulated in the presence of protein transport inhibitor, GolgiStop™ GolgiPlug™ with the same peptide pools as the ELISpots. Media alone and phorbol 12-myristate 13-acetate (PMA) and ionomycin stimulations were used as negative and positive controls respectively. To test for degranulation of cells, anti-CD107a antibody was also added during stimulation. After 6 hrs, cells were washed and stained with LIVE/DEAD violet. Surface staining was then added containing anti-CD4, anti-CD8, anti-CD62L and anti-CD44. After 30 minute incubation, cells were spun, washed, and fixed using the CytoPerm CytoWash kit following manufacturer's protocol. Intracellular staining was then prepared using anti-IFNγ, anti-TNFα, anti-IL2, and anti-CD3.

All data was collected on a modified LSRII flow cytometer followed by analysis with FlowJo software.

x. ELISA

Binding titers to gp120 were determined by coating plates with 1 ug/ml of BG505 gp120 overnight in PBS. After washing, plates were blocked with 5% skim milk in PBS with 1% newborn calf serum (NBS) and 0.2% Tween for 1 hour at RT. Serum was serially diluted, added to plates and incubated at 37o for 1 hour. Antigen and species specific IgG was then detected with secondary anti-mouse, rabbit or NHP HRP antibody. Plate were developed for 5 minutes with TMB and stopped with 2N H2SO4.

Binding titers to trimer were determined by coating plates with 2 ug/ml of recombinant PGT128 antibody overnight in PBS. After washing, plates were blocked with 5% skim milk in PBS with 1% newborn calf serum (NBS) and 0.2% Tween for 1 hour at RT. Recombinant trimer was added at 4 ug/ml for 2 hours at RT. Serum was serially diluted, added to plates and incubated at 37o for 1 hour. Antigen and species specific IgG was then detected with secondary anti-mouse, rabbit or NHP HRP antibody. Plate were developed for 5 minutes with TMB and stopped with 2N H2SO4.

Competition ELISAs were performed using a similar protocol for trimer specific antibodies. Serum was diluted at a 1:60 concentration and added to plates for 1 hour at 37°. Recombinant 1 TA or 12N were then added at a set concentration to yield the EC70 binding. Competition was then determined by detecting with a secondary anti-human HRP antibody. Plate were developed for 5 minutes with TMB and stopped with 2N H2SO4. Percent competition was determined using the following equation ((1−(OD450 EC70−sample OD))*100.

xi. Neutralization Assay

Pseudotype viruses were titered to yield 1500, 000 RLU after 48 h of infection with Tzm-Bl cells. Mouse serum was heat inactivated for 15 minutes at 56° and NHP serum was inactivate for 30 minutes. Serum or monoclonal antibody controls were serially diluted and incubated with virus before adding 10,000 Tzm-Bl cells per well with dextran. Forty-eight hours after incubation, media was removed and cells were lysed using BriteLite luciferase reagent. Serum concentration/titer was determined for 50% virus neutralization (IC50).

xii. Statistics

All statistics and calculations were performed using GraphPad Prism 7.0. EC50 and EC70 concentrations were calculated using a non-linear regression model. IC50 values were computed with a non-linear regression model of percentage neutralization vs log reciprocal serum dilution. All statistical test were calculated in GraphPad using p<0.05 as significant. In most cases a modified one-way ANOVA was performed and corrected for multiple comparisons.

### 2. Results/Discussion

i. Protein vs DNA immunization of Trimer immunogens.

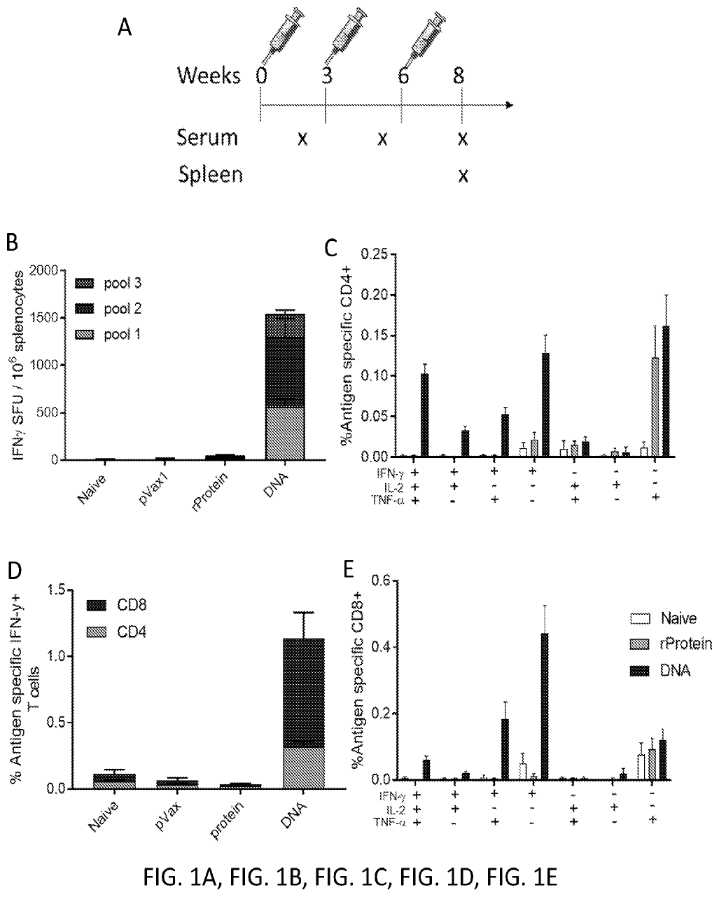

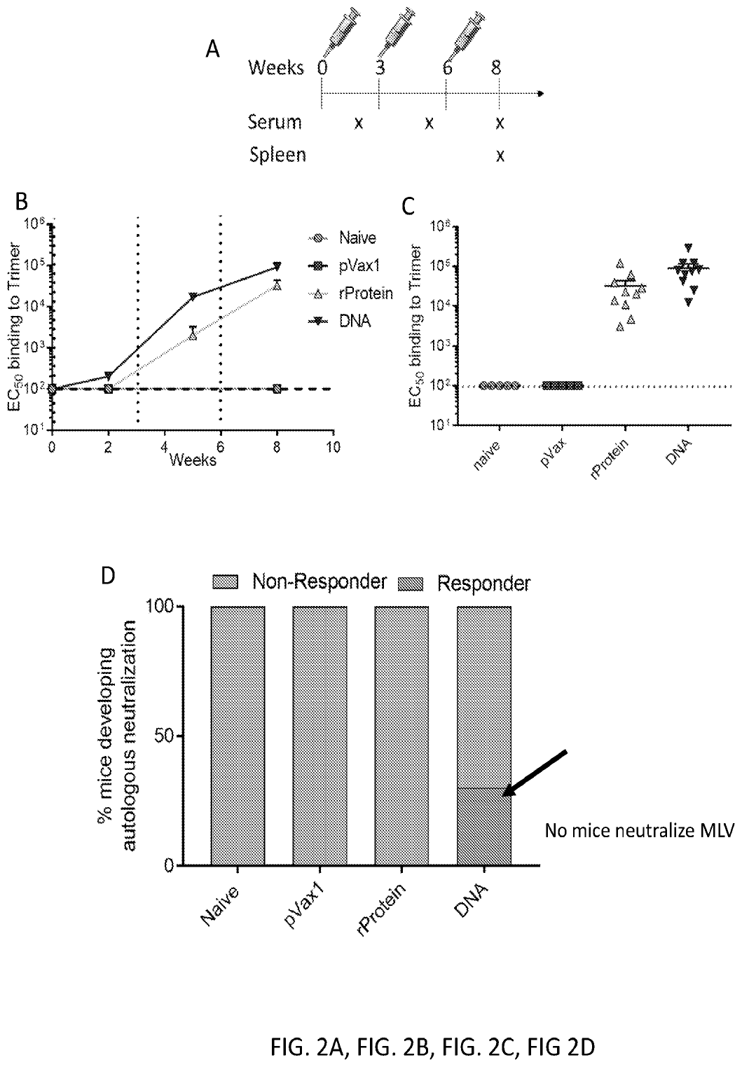

In order to first explore the ability of DNA encoded native like HIV-1 Envelope trimers, immune responses were compared in mice receiving either recombinant trimer or EP-DNA. Mice were delivered the same dose (25 ug) of either DNA or protein delivered at weeks 0, 3, and 6 (FIG. 1A). Two weeks after final immunization, mice were sacrificed and cellular responses were determined using overlapping peptides for WT BG505 Env sequence. The mice immunized with DNA alone were able to induce strong T cell responses especially compared to the recombinant protein immunized animals. These antigen specific T cells were able to recognize peptides from across the antigen (FIG. 1B) and were both CD4+ and CD8+ T cells (FIG. 1D). Additionally, both CD4+ and CD8+ antigen specific T cells were able to express multiple cytokines including triple positive cells (expressing IFN-γ, TNFα, and IL-2) (FIGS. 1C and 1E). The ability of these mice to induce antibodies which recognize the HIV-1 native like trimer were also investigated. Humoral responses were determined post dose 1, 2, and 3 and at all time points, DNA was able to induce higher binding antibodies (FIG. 2B, 2C). Two weeks after the final immunization, there was still a trend to higher binding antibodies to trimer in the DNA group, but this difference was not significant.

Previously, groups have demonstrated that through recombinant stabilized native like trimer protein can induce autologous Tier 2 neutralizing antibody titers in larger animals (rabbits and NHPS), these responses have not been observed in mice. The dogma was that though mice could induce strong binding antibodies, develop good Tfh responses and germinal centers, they did not have the BCRs to induce an autologous neutralizing antibody response. In light of this, we decided to investigate if our DNA encoded trimer was able to induce autologous neutralizing antibodies to BG505. In the naïve, pVax backbone control and protein only immunize mice, neutralizing titers were not observed against BG505 pseudotype virus. However, in 3 out of 10 (or 30%) of mice immunized with DNA no neutralizing antibody titers were observed (FIG. 2D). In all cases, no mice were able to neutralize the MLV control virus to prevent any non-specific neutralization effects.

ii. Improving the Antibody Immune Response by Increasing the Interval Between Boost.

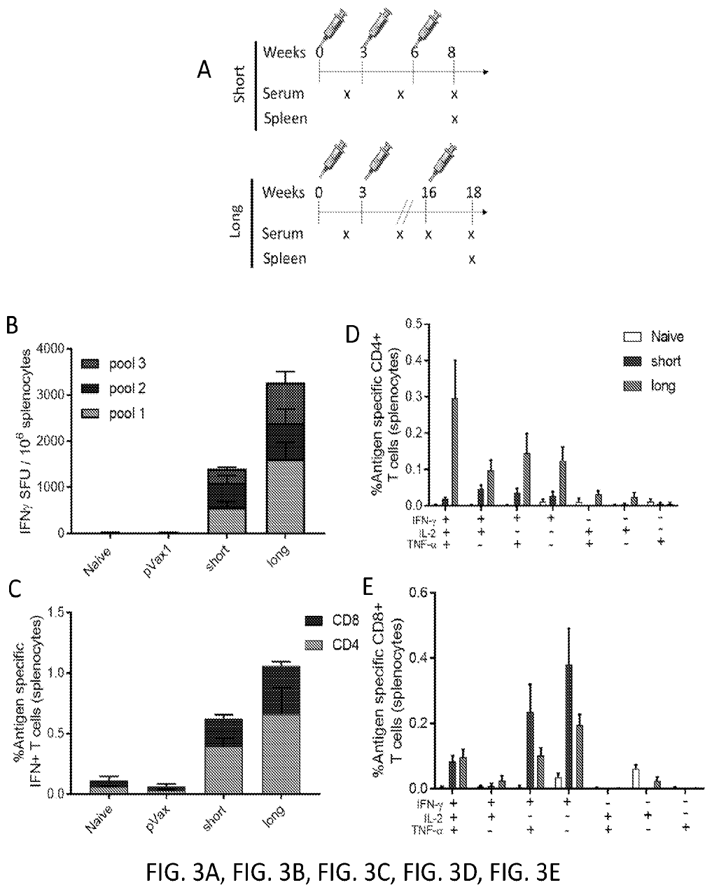

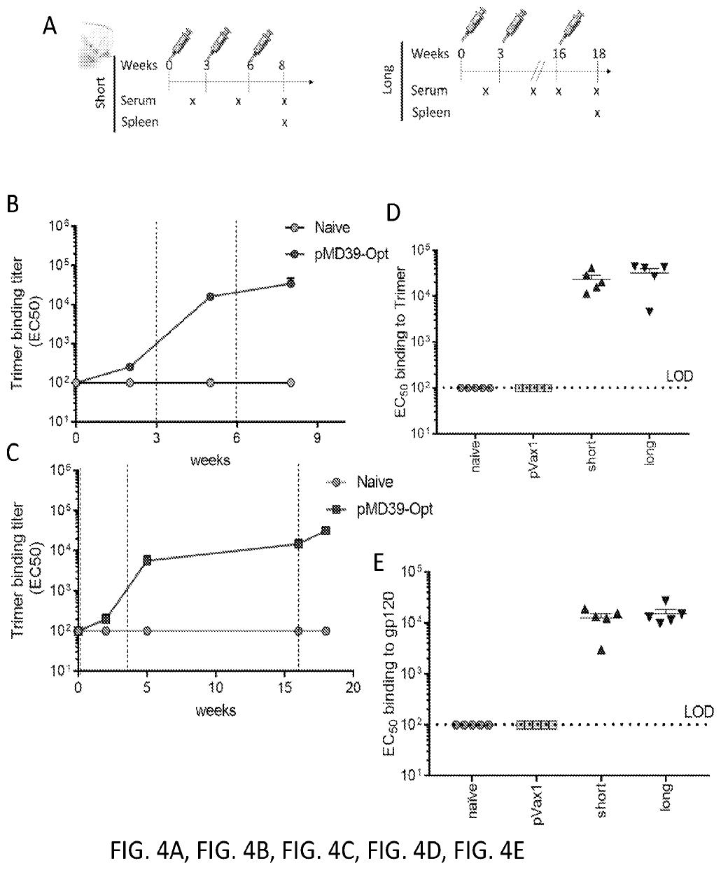

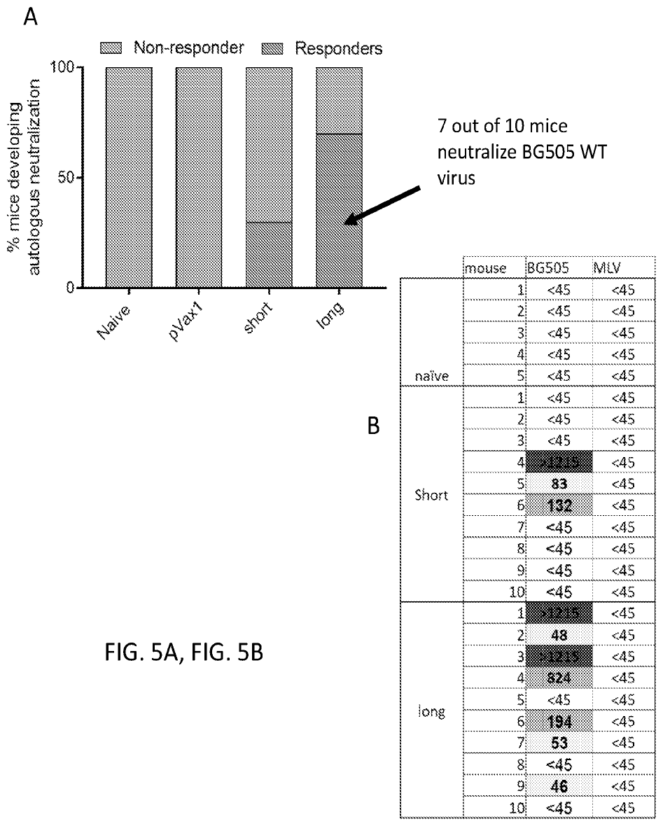

It has been previously demonstrated that longer intervals between vaccinations can yield a superior antibody responses by allow time for somatic hypermutation and affinity maturation to occur. Lengthening the interval between the second and third immunization could increase our antibody responses. Mice were immunized with 25 ug of DNA encoding the native like trimer followed by EP at weeks 0, 3, 6 or weeks 0, 3, 16. In both regimens, mice were euthanized two weeks after final immunization. Mice immunized with the longer interval induced higher T cell responses compared to the shorter immunization schedule (FIG. 3A). In both cases, these T cells were both CD4+ and CD8+ in specificity. Interesting, the longer interval induced stronger CD4 poly-functionally (FIG. 3B) whereas the shorter immunization induce more CD8 poly-functionality (FIG. 3E). Both schedules were able to induce strong antibody responses which recognized by recombinant trimer and gp120 monomer (FIG. 4). It is important to note that there is not much of a decline in antibody titers between week 5 (2 weeks post dose 2) and week 16 (pre dose 3) for the longer interval (FIG. 4C). Extending the interval improves the neutralization responses. In the longer interval group, 7 out of 10 mice developed neutralization titers compared to 3 out of 10 for the short immunization (FIG. 5). This indicates that a longer interval improves the antibody neutralization capacity.

iii. Exploring Additional Constructs—Making Improvements

Though the pMD39-Opt construct was able to induce autologous tier 2 neutralizing antibody titers, making improvements on this construct as well as further defining which type of construct worked best for DNA plasmid delivery was investigated. Currently, MD39 relies on furin for cleavage. By including different linkers, a trimer can be encoded which is no longer dependent on furin. Additionally, immunogens can be encoded which have the bottom of the trimer masked to prevent off target bottom binding antibodies by including mutations to add in a glycan. A string of monomers (trimer strings) can also be encoded which could allow for better folding and proper assembly when multiple Envs are expressed in the same cell. Adding a transmembrane domain and physically linking the trimer to the membrane could change the immune responses.

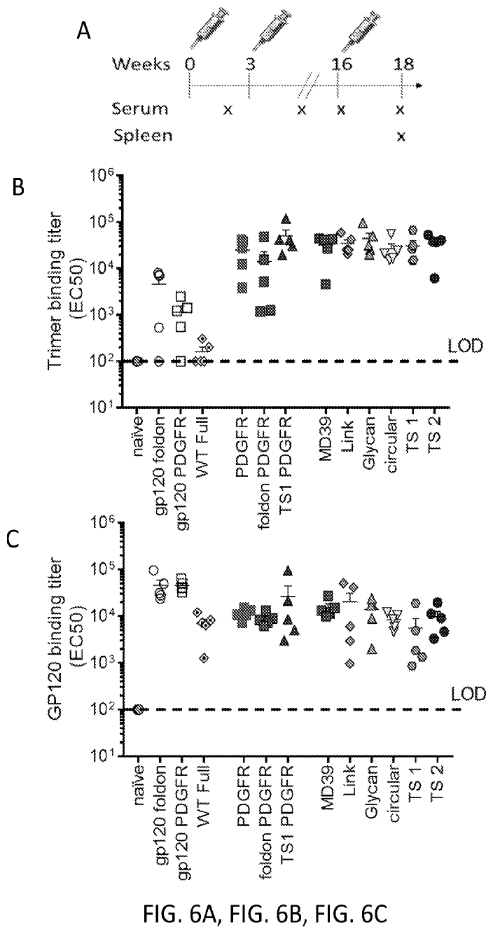

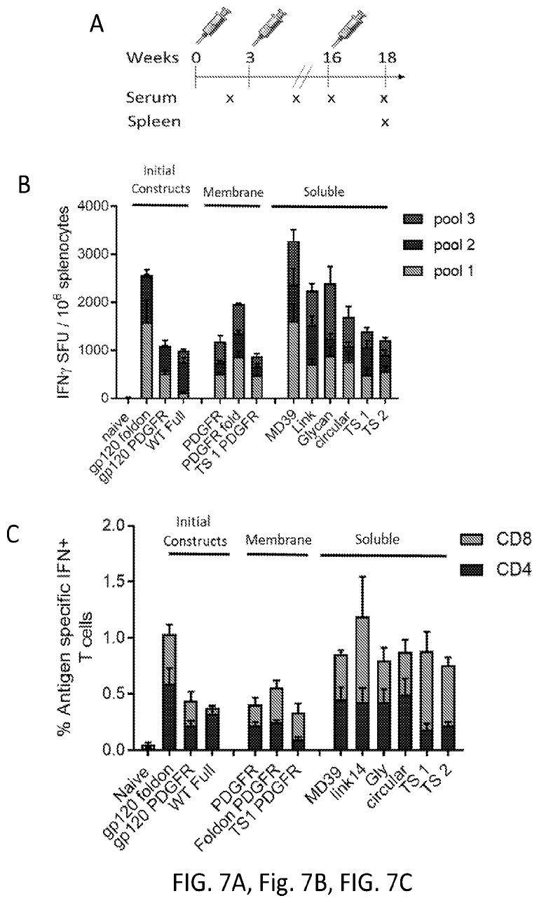

Binding titers to gp120 and HIV-1 Env trimer were explored. Previous iterations of DNA encoded Envs, WT and gp120 foldons, were able to induce good binding titers to gp120 monomer but weak and spotty responses to trimer. When the disclosed DNA encoded trimers were used, higher binding titers to trimer and slight lower binding responses to gp120 monomer was observed (FIG. 6). There was no difference in terms of binding between any of the trimer constructs. Cellular responses of these immunogen were also explored. All mice were able to induce significantly higher antigen specific T cell responses compared to naïve mice (FIG. 7). There was a decrease in cellular responses for the trimer string antigens and the membrane bound antigens. However, all constructs were able to induce both CD4+ and CD8+ T cells.

iv. V3 Responses Induced by DNA Encoded Trimer Immunogens.

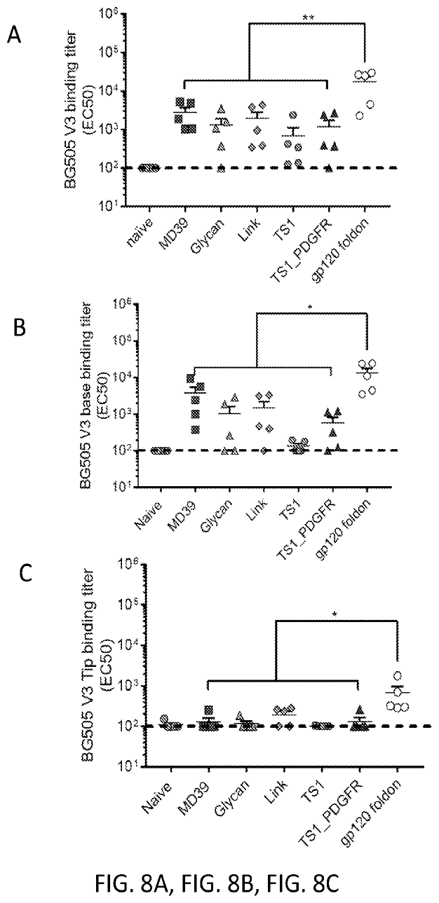

In gp120, the V3 loop is exposed and folded out. In native like trimers, this loop is buried and is not exposed to the immune system. Thus, antibodies binding to V3 can be an indirect measure of proper antigen folding. The reactivity of a subset of serum from the DNA immunized mice were explored. Compared to control gp120 foldon immunized mice, a significant decrease in the V3 binding antibodies was seen (FIG. 8). As a control, ELISAs were performed on scrambled peptides to ensure this binding was specific. Thus, the DNA encoded trimers are folding properly.

v. DNA Encoded Modifications Limit Bottom Binding Antibodies

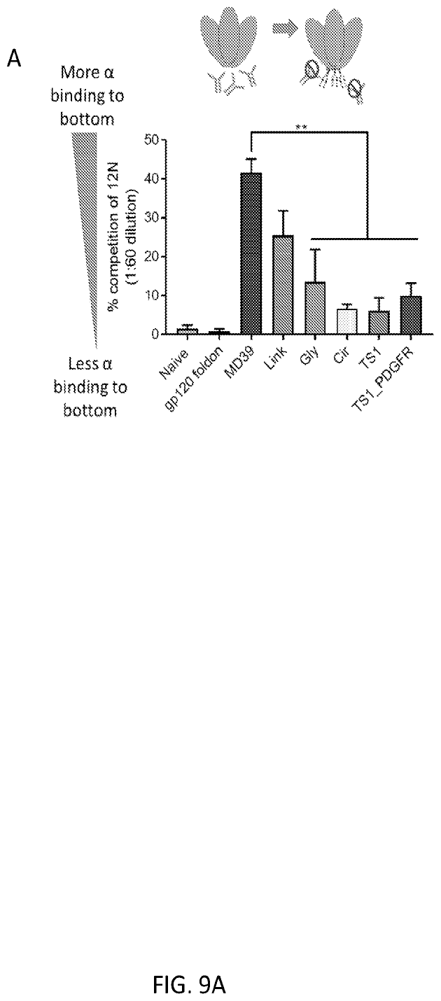

In the pMD39-OPT construct, the base of the trimer is exposed due to secretion. Normally, in the context of infection, this region is hidden by the transmembrane region of the Env. However, this immunodominant region is exposed when it is expressed as a soluble trimer. This region can be “hidden” from the immune system by adding in different glycans, creating different linker locations or attaching it to the membrane. In order to explore if these modifications were able to prevent reactivity, a competition ELISA was performed using a known monoclonal that binds to the bottom of the trimer. Compared to base pMD39_opt, the addition of glycans, linkers or linking it to the membrane, significantly decreased the amount of antibodies that competed for binding with 12N (FIG. 9). In other words, these mice induces less bottom binding antibodies. This is an important demonstration of how different modifications encoded in DNA can translate to in vivo immune responses. Additionally it is an indirect demonstration that glycan sites can be encoded and obtain those glycosylation events.

vi. Neutralization of Autologous Tier 2 BG505 Virus

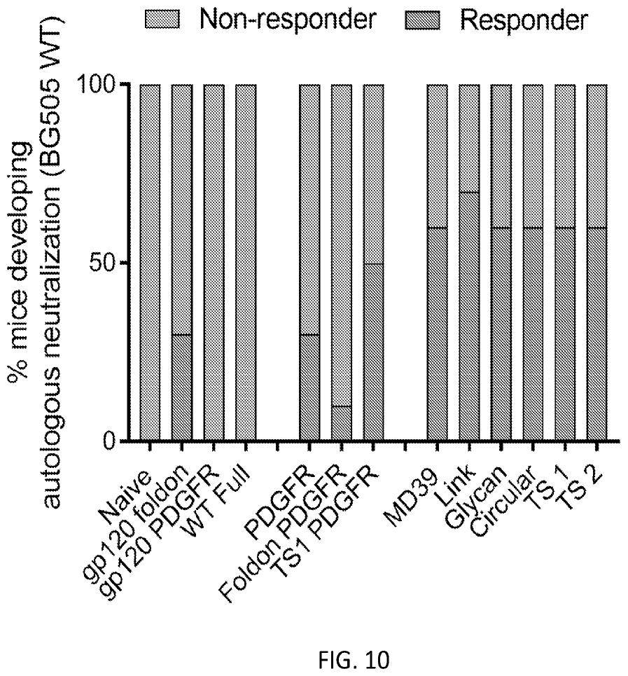

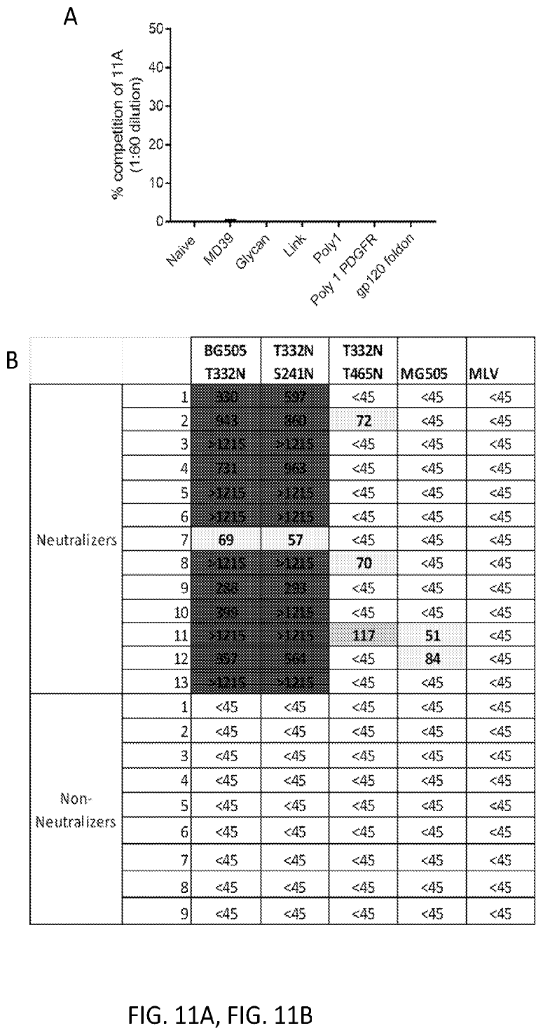

If autologous neutralizing antibody titers were induced with the different forms of DNA encoded structural immunogens was investigated. The best membrane bound immunogen was the trimer string_PDGFR that induced 50% of mice inducing autologous neutralizing titers. The soluble antigens induce between 60-70% of autologous neutralizing antibody titers. There was no neutralization with MLV control virus. Thus, across multiple antigens and different iteration we are able to get tier 2 autologous neutralizing antibody titers in mice (FIG. 10). Where these antibodies bound and neutralize the virus was determined. There is a monoclonal antibody which binds to the epitope which is dominant in rabbits immunized with a similar protein antigens. This antibody binds to a hole in the glycans on HIV Env at the 241 position. It is called 11A. A competition ELISA can be used to determine if the serum is binding to this epitope. Serum from mice immunized at wk 0, 3, 16 (wk 18 serum was used) for the competition with 1 TA. There was no competition with 11 A from the mouse serum (FIG. 11A). Mutations were made to the BG505 virus to add in a glycan at this site (S241N mutation). By adding in this mutation, 11A was prevented from neutralizing the pseudotype virus thus demonstrating that the virus is in fact glycosylated at this position. The control in this experiment is PDGM1400 which is a broadly neutralizing antibody and is able to neutralize both the parent and mutated virus to the similar extent. When using serum from mice that are able to induce neutralization titers to BG505 T332N vs those which did not, no decrease in neutralization capacity with the S241N mutation was observed, indicating that the mouse neutralizing response is not targeting this region (FIG. 11).

The next epitope tested was the C3/465 region of the Envelop. This is the dominant neutralizing epitope response in NHPs and is in 25% of rabbits. A virus was produced which encodes the T465N (adding a glycan at this position). The majority of antibody responses are removed and all are decreased in titers (FIG. 11B). Furthermore, the maternal strain (MG505) which was the transmitting virus into the baby girl (BG505) for which this initial Env sequence was isolated, is closely related (17AA differences) (FIG. 11B). One of these is in the region previously observed in NHPs (I396N). This could explain why MG505 is not neutralized by the mouse serum.

vii. Rabbits Immunized with DNA Encoded Trimers Induce Trimer Specific Binding Antibodies and Some Autologous Tier 2 Neutralizing Titers

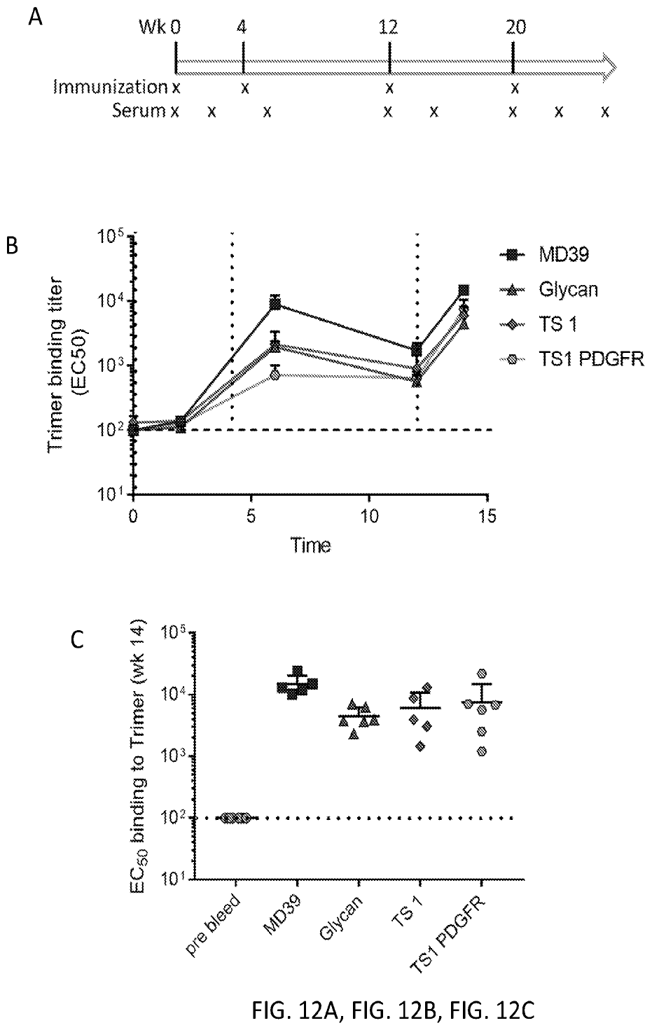

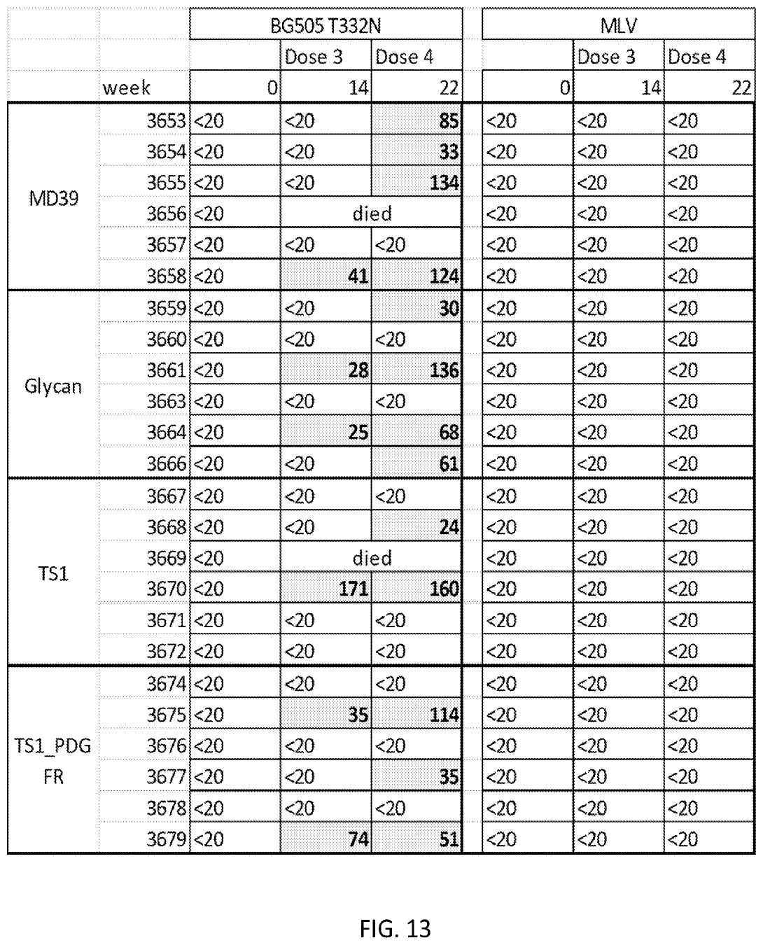

After downselection in mice, four different DNA encoded trimers were moved into larger animal models—the rabbit. Rabbits were immunized with either 1-2 mg of DNA based on the molar amount delivered to two sites ID with CELLECTRA 3P at wk 0, 4, 12, 20 (FIG. 12). Trimer specific antibody responses were detected with complete seroconverstion post second immunization. These responses were slightly higher with pOpt-MD39 compared to the other DNA encoded immunogens. This could be due to increased bottom binding antibodies. There are some neutralization titers post third immunization against autologous virus (BG505 T332N) which are further boosted after forth immunization (FIG. 13). There was limited to no non-specific (MLV) neutralizing titers (FIG. 13).

viii. NHPs Immunized with DNA Encoded Trimers Induce Trimer Specific Binding Antibodies and Antigen Specific T Cell Responses

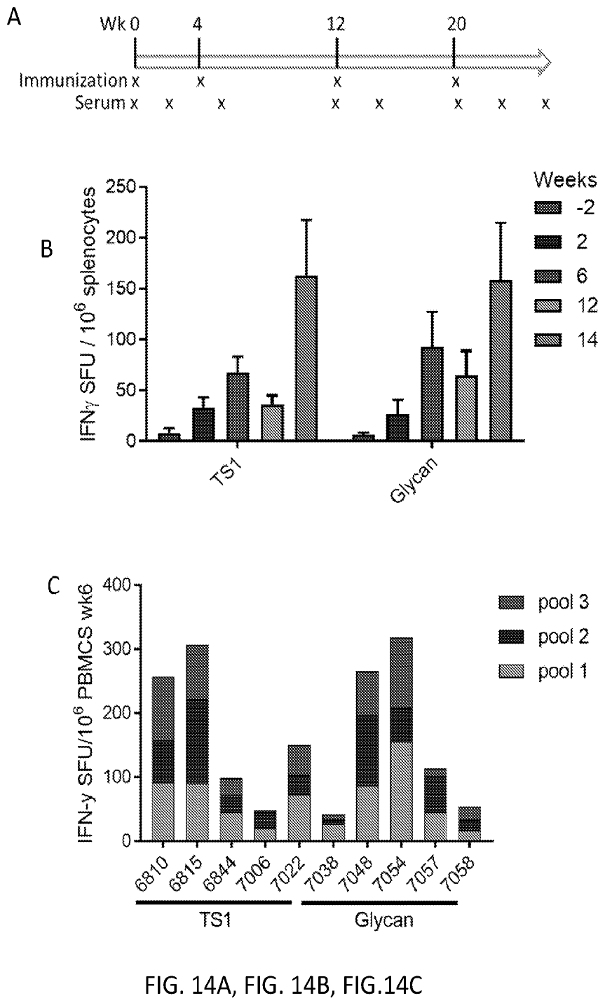

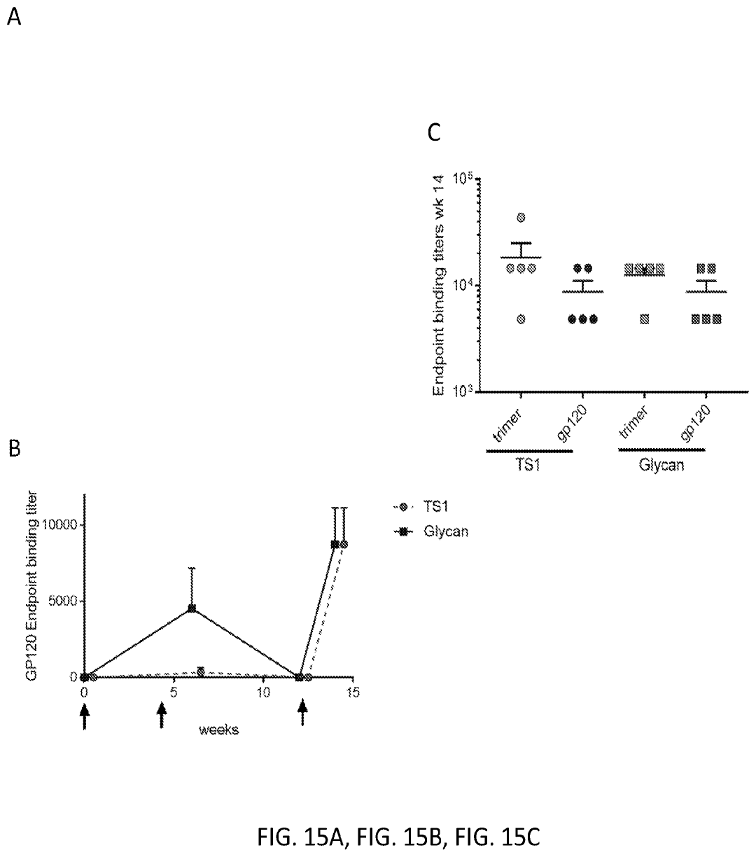

The ability for DNA encoded native like trimers to induce responses was also studied in NHPs. NHPs were immunized with 2 mgs of DNA delivered to two sites ID with CELLECTRA 3Pat weeks 0, 4, 12, and 20 (FIG. 14). Antigen specific T cells were observed as early as post first dose and subsequentially boosted after each immunization (FIG. 14B). Additionally, antigen specific T cells recognized the entire length of the protein as seen in responses to every peptide pool (FIG. 14C). These NHPS are able to induce stronger trimer specific antibody titers compared to gp120 specific responses post dose 2 (FIG. 15). It is too early to determine if these NHPS will develop autologous neutralizing antibody titers.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Cohen K W, Frahm N. Current views on the potential for development of a HIV vaccine. Expert opinion on biological therapy. 2017; 17(3):295-303. doi: 10.1080/14712598.2017.1282457. PubMed PMID: 28095712; PubMed Central PMCID: PMCPMC5538888.
2. Pancera M, Changela A, Kwong PD. How HIV-1 entry mechanism and broadly neutralizing antibodies guide structure-based vaccine design. Current opinion in HIV and AIDS. 2017; 12(3):229-40. doi: 10.1097/COH.0000000000000360. PubMed PMID: 28422787; PubMed Central PMCID: PMCPMC5557343.
3. Rubens M, Ramamoorthy V, Saxena A, Shehadeh N, Appunni S. HIV Vaccine: Recent Advances, Current Roadblocks, and Future Directions. J Immunol Res. 2015; 2015:560347. doi: 10.1155/2015/560347. PubMed PMID: 26579546; PubMed Central PMCID: PMCPMC4633685.
4. Pollara J, Easterhoff D, Fouda G G. Lessons learned from human HIV vaccine trials. Current opinion in HIV and AIDS. 2017; 12(3):216-21. doi: 10.1097/COH.0000000000000362. PubMed PMID: 28230655; PubMed Central PMCID: PMCPMC5389590.
5. Torrents de la Pena A, Julien J P, de Taeye S W. Garces F, Guttman M, Ozorowski G, et al. Improving the Immunogenicity of Native-like HIV-1 Envelope Trimers by Hyperstabilization. Cell Rep. 2017; 20(8):1805-17. doi: 10.1016/j.celrep.2017.07.077. PubMed PMID: 28834745; PubMed Central PMCID: PMCPMC5590011.
6. Sanders R W, Moore J P. Native-like Env trimers as a platform for HIV-1 vaccine design. Immunol Rev. 2017; 275(1):161-82. doi: 10.1111/imr.12481. PubMed PMID: 28133806; PubMed Central PMCID: PMCPMC5299501.
7. Medina-Ramirez M, Garces F, Escolano A, Skog P, de Taeye S W, Del Moral-Sanchez I. et al. Design and crystal structure of a native-like HIV-1 envelope trimer that engages multiple broadly neutralizing antibody precursors in vivo. The Journal of experimental medicine. 2017; 214(9):2573-90. doi: 10.1084/jem.20161160. PubMed PMID: 28847869; PubMed Central PMCID: PMCPMC5584115.
8. Kulp D W, Steichen J M, Pauthner M, Hu X, Schiffner T, Liguori A, et al. Structure-based design of native-like HIV-1 envelope trimers to silence non-neutralizing epitopes and eliminate CD4 binding. Nature communications. 2017; 8(1):1655. doi: 10.1038/s41467-017-01549-6. PubMed PMID: 29162799; PubMed Central PMCID: PMCPMC5698488.
9. Pauthner M G, Nkolola J P, Havenar-Daughton C, Murrell B, Reiss S M, Bastidas R, et al. Vaccine-Induced Protection from Homologous Tier 2 SHIV Challenge in Nonhuman Primates Depends on Serum-Neutralizing Antibody Titers. Immunity. 2019; 50(1):241-52 e6. doi: 10.1016/j.immuni.2018.11.011. PubMed PMID: 30552025; PubMed Central PMCID: PMCPMC6335502.
10. Bianchi M, Turner H L, Nogal B, Cottrell C A, Oyen D, Pauthner M, et al. Electron-Microscopy-Based Epitope Mapping Defines Specificities of Polyclonal Antibodies Elicited during HIV-1 BG505 Envelope Trimer Immunization. Immunity. 2018; 49(2):288-300 e8. doi: 10.1016/j.immuni.2018.07.009. PubMed PMID: 30097292; PubMed Central PMCID: PMCPMC6104742.
11. Pauthner M, Havenar-Daughton C, Sok D, Nkolola J P, Bastidas R, Boopathy A V, et al. Elicitation of Robust Tier 2 Neutralizing Antibody Responses in Nonhuman Primates by HIV Envelope Trimer Immunization Using Optimized Approaches. Immunity. 2017; 46(6):1073-88 e6. doi: 10.1016/j.immuni.2017.05.007. PubMed PMID: 28636956; PubMed Central PMCID: PMCPMC5483234.
12. Dey A K, Cupo A, Ozorowski G, Sharma V K, Behrens A J, Go E P, et al. cGMP production and analysis of BG505 SOSIP.664, an extensively glycosylated, trimeric HIV-1 envelope glycoprotein vaccine candidate. Biotechnol Bioeng. 2018; 115(4):885-99. doi: 10.1002/bit.26498. PubMed PMID: 29150937; PubMed Central PMCID: PMCPMC5852640.
13. Ringe R P, Ozorowski G, Yasmeen A, Cupo A, Cruz Portillo V M, Pugach P, et al. Improving the Expression and Purification of Soluble, Recombinant Native-Like HIV-1 Envelope Glycoprotein Trimers by Targeted Sequence Changes. Journal of virology. 2017; 91(12). doi: 10.1128/JVI.00264-17. PubMed PMID: 28381572; PubMed Central PMCID: PMCPMC5446630.
14. Patel A, Reuschel E L, Kraynyak K A, Racine T, Park D H, Scott V L, et al. Protective Efficacy and Long-Term Immunogenicity in Cynomolgus Macaques by Ebola Virus Glycoprotein Synthetic DNA Vaccines. The Journal of infectious diseases. 2018. doi: 10.1093/infdis/jiy537. PubMed PMID: 30304515.
15. Morrow M P, Kraynyak K A, Sylvester A J, Dallas M, Knoblock D, Boyer J D, et al. Clinical and Immunologic Biomarkers for Histologic Regression of High-Grade Cervical Dysplasia and Clearance of HPV16 and HPV18 after Immunotherapy. Clinical cancer research: an official journal of the American Association for Cancer Research. 2018; 24(2):276-94. doi: 10.1158/1078-0432.CCR-17-2335. PubMed PMID: 29084917.
16. Tebas P, Roberts C C, Muthumani K, Reuschel E L, Kudchodkar S B, Zaidi F I, et al. Safety and Immunogenicity of an Anti-Zika Virus DNA Vaccine-Preliminary Report. The New England journal of medicine. 2017. doi: 10.1056/NEJMoa1708120. PubMed PMID: 28976850.
17. Morrow M P, Kraynyak K A, Sylvester A J, Shen X, Amante D, Sakata L, et al. Augmentation of cellular and humoral immune responses to HPV16 and HPV18 E6 and E7 antigens by VGX-3100. Mol Ther Oncolytics. 2016; 3:16025. doi: 10.1038/mto.2016.25. PubMed PMID: 28054033; PubMed Central PMCID: PMCPMC5147865.
18. Trimble C L, Morrow M P, Kraynyak K A, Shen X, Dallas M, Yan J, et al. Safety, efficacy, and immunogenicity of VGX-3100, a therapeutic synthetic DNA vaccine targeting human papillomavirus 16 and 18 E6 and E7 proteins for cervical intraepithelial neoplasia 2/3: a randomised, double-blind, placebo-controlled phase 2b trial. Lancet. 2015; 386(10008):2078-88. doi: 10.1016/S0140-6736(15)00239-1. PubMed PMID: 26386540; PubMed Central PMCID: PMCPMC4888059.
19. Khoshnejad M, Patel A, Wojtak K, Kudchodkar S B, Humeau L, Lyssenko N N, et al. Development of Novel DNA-Encoded PCSK9 Monoclonal Antibodies as Lipid-Lowering Therapeutics. Molecular therapy: the journal of the American Society of Gene Therapy. 2019; 27(1):188-99. doi: 10.1016/j.ymthe.2018.10.016. PubMed PMID: 30449662; PubMed Central PMCID: PMCPMC6319316.
20. Xu Z, Wise M C, Choi H, Perales-Puchalt A, Patel A, Tello-Ruiz E, et al. Synthetic DNA delivery by electroporation promotes robust in vivo sulfation of broadly neutralizing anti-HIV immunoadhesin eCD4-Ig. EBioMedicine. 2018; 35:97-105. doi: 10.1016/j.ebiom.2018.08.027. PubMed PMID: 30174283; PubMed Central PMCID: PMCPMC6161476.
21. Wang Y, Esquivel R, Flingai S, Schiller Z A, Kern A, Agarwal S, et al. Anti-OspA DNA-Encoded Monoclonal Antibody Prevents Transmission of Spirochetes in Tick Challenge Providing Sterilizing Immunity in Mice. The Journal of infectious diseases. 2018. doi: 10.1093/infdis/jiy627. PubMed PMID: 30476132.
22. Patel A, Park D H, Davis C W, Smith T R F, Leung A, Tiemey K, et al. In Vivo Delivery of Synthetic Human DNA-Encoded Monoclonal Antibodies Protect against Ebolavirus Infection in a Mouse Model. Cell Rep. 2018; 25(7):1982-93 e4. doi: 10.1016/j.celrep.2018.10.062. PubMed PMID: 30428362; PubMed Central PMCID: PMCPMC6319964.
23. Patel A, DiGiandomenico A, Keller A E, Smith T R F, Park D H, Ramos S, et al. An engineered bispecific DNA-encoded IgG antibody protects against Pseudomonas aeruginosa in a pneumonia challenge model. Nature communications. 2017; 8(1):637. doi: 10.1038/s41467-017-00576-7. PubMed PMID: 28935938; PubMed Central PMCID: PMCPMC5608701.
24. Elliott S T C, Kallewaard N L, Benjamin E, Wachter-Rosati L, McAuliffe J M, Patel A, et al. DMAb inoculation of synthetic cross reactive antibodies protects against lethal influenza A and B infections. NPJ Vaccines. 2017; 2:18. doi: 10.1038/s41541-017-0020-x. PubMed PMID: 29263874; PubMed Central PMCID: PMCPMC5627301.

The disclosure relates to compositions, pharmaceutical compositions, and cells comprising nucleic acid molecules such as plasmids comprising at least a first expressible nucleic acid sequence that comprises any one or combination of sequences in Table Y or any one or combination of nucleic acid sequences that encode an amino acid sequence from Table Y. The disclosure relates to compositions, pharmaceutical compositions, and cells comprising fragments of those sequences or mutants of those sequences that comprise at least 70%, 80%, 81%, 82%, 83%, 84%, 85%, 86%, 87%, 88%, 89%, 90%, 91%, 92%, 93%, 94%, 95%, 96%, 97%, 98%, or 99% sequence identity to nucleic acid sequence fragments at least 20, 30, 40, 50, 60, 70, 80, 90, 100, 125, 150, 175, 200, 250, 275, 300, 350, 400, 450, 500 or more nucleic acids of the sequence of Table Y.

In some embodiments, the disclosure relates to pharmaceutical compositions or cells comprising such pharmaceutical compositions comprising a plasmid disclosed herein with at least one expressible nucleic acid that is any one or combination of sequences in Table Y or any one or combination of nucleic acid sequences that encode an amino acid sequence from Table Y, or pharmaceutically salts thereof, or any sequence comprising at least 70%, 80%, 81%, 82%, 83%, 84%, 85%, 86%, 87%, 88%, 89%, 90%, 91%, 92%, 93%, 94%, 95%, 96%, 97%, 98%, or 99% sequence identity to those sequences identified in Table Y.

### TABLE Y of SEQUENCES

BG505 MD39 based sequences

Parts of sequences
Leader sequences
IgE
MDWTWILFLVAAATRVHS (SEQ ID NO: 7)
MD39 atggactggacatggattctgttcctggtcgctgccgctacaagagtgcattcc (SEQ ID NO: 2)
CPG9.2 atggattggacttggattctgttcctggtcgcagcagccacacgagtgcatagc (SEQ ID NO: 3)
Cleavage sites
Furin
RRRRRR (SEQ. ID NO: 236)
Cggcgcaggagacggcgc (SEQ ID NO: 237)
Linkers
Link 14
SHSGSGGSGSGGHA (SEQ ID NO: 14)
tctcacagcggctccggcggctctggcagcggcggccacgcc
GS linkers
(SEQ ID NO: 17)
(SEQ ID N0: 15)
CPG9.2
GGNSSG (SEQ ID NO: 20)
Gggggaaatagtagcggc (SEQ ID NO: 18)
GGNGSGGGSGSGGNGSSG (SEQ ID NO: 23)
Ggcggcaacggcagcggcggcggcagcggctccggcggcaacggctctagcggc (SEQ ID NO: 21)
PDGFR linker between trimer or TS1 and PDGFR
GGGSGGSGGSGGSGGSGGS (SEQ ID NO: 26)
Ggaggaggaagcgggggaagcgggggaagcggaggaagcgggggaagcgggggaagc (SEQ ID NO: 24)
Foldon PDGFR linkers
GGGSGGSGGG (SEQ ID NO: 29)
Ggaggaggaagcgggggaagcggcggcggc (SEQ ID NO: 27)
GGSGGSGGSGGS (SEQ ID NO: 32)
Gggggaagcggaggaagcgggggaagcgggggaagc (SEQ ID NO: 29)
3BVE
GSG
ggaagcggc
I3_1
GGSGSGGSGG (SEQ ID NO: 35)
Ggcggcagcggcagcggcgggagcggagga (SEQ ID NO: 33)
I3_2
GGSDMRKDAERRFDKFVEAAKNKFDKFKAALRKGDIKEERRKDMKKLARKEAEQARRAVRNRLSELLSKINDMPIT
NDQKKLMSNDVLKFAAEAEKKIEALAADAEGGSGS (SEQ ID NO: 38)
Ggagggagcgatatgagaaaggacgccgagagacggtttgataagttcgtggaggctgctaagaataagtttgacaagtttaaggctgccctg
cggaagggcgacatcaaggaggagaggagaaaggatatgaagaagctggcaaggaaggaggcagagcaggcaaggagggccgtgaggaa
cagactgagcgagctgctgtccaagatcaacgacatgcccatcaccaatgatcagaagaagctgatgtctaatgacgtgctgaagttcgccgca
gaagccgaaaagaagattgaagccctggcagcagacgccgaaggaggaagcgggagc (SEQ ID NO: 36)
LS_1
GGSSGKSLVDTVYALKDEVQELRQDNKKMKKSLEEEQRARKDLEKLVRKVLKNMNDGGSSG (SEQ ID NO: 41)
Gggggctctagcgggaaaagtctggtggataccgtctatgctctgaaagatgaggtgcaggaactgaggcaggacaacaaaaagatgaagaa
gagcctggaggaggagcagagggccagaaaggacctggaaaaactggtgcggaaagtgctgaaaaacatgaatgacggagggagtagcgg
g (SEQ ID NO: 39)
LS_2
GGSSGADPKKVLDKAKDQAENRVRELKQKLEELYKEARKLDLTQEMRRKLELRYIAAMLMAIGDIYNAIRQAKQEA
DKLKKAGLVNSQQLDELKRRLEELKEEASRKARDYGREFQLKLEYGGGSGSGSG (SEQ ID NO: 44)
Gggggctctagcggggcagacccaaagaaagtgctggataaggcaaaggatcaggcagagaatagagtgagagaactgaaacagaaactg
gaggaactgtataaggaggcccggaagctggacctgacccaggagatgaggagaaagctggagctgcgctacatcgccgccatgctgatggc
catcggcgacatctataacgccatcaggcaggccaagcaggaggccgataagctgaagaaggccggcctggtgaatagccagcagctggacg
agctgaagcggcgcctggaggagctgaaggaggaggcctccaggaaggccagagattatgggcgggaatttcagctgaaactggagtatggc
ggcggaagcggaagcgggagcggg (SEQ ID NO: 42)
QB_1
GGSSGGTDVGAIAGKANEAGQGAYDAQVKNDEQDVELADHEARIKQLRIDVDDHESRITANTKAITALNVRVTTA
EGEIASLQTNVSALDGRVTTAENNISALQADYVSGGSSGSG (SEQ ID NO: 47)
Ggaggctcttcaggcggcacagacgtgggggcaatcgctggaaaggctaacgaggctggacagggggcttatgatgctcaggtcaaaaacga
cgagcaggatgtggagctggccgaccacgaggccaggatcaagcagctgagaatcgatgtggacgatcacgagtctcggatcaccgccaaca
caaaggccatcacagccctgaatgtgcgcgtgaccacagcagagggagagatcgcatccctgcagaccaacgtgagcgccctggacggaagg
gtgaccacagcagagaacaatatctccgccctgcaggcagattacgtgagcggcggcagctccggctccgga (SEQ ID NO: 45)
QB_2
GGSGSGGSSGPHMIAPGHRDEFDPKLPTGEKEEVPGKPGIKNPETGDVVRPPVDSVTKYGPVKGDSIVEKEEIPFEK
ERKFNPDLAPGTEKVTREGQKGEKTITTPTLKNPLTGEIISKGESKEEITKDPINELTEWGPETGGSGSGGSS
ggaggctctggaagcgggggaagtagcggacctcacatgattgctccaggacatcgggacgagtttgaccctaagctgccaacaggcgagaaa
gaagaggtgccaggcaagcccggcatcaagaaccctgagacaggcgacgtggtgaggccccctgtggattctgtgacaaagtacggcccagtg
aagggcgacagcatcgtggagaaggaggagatccccttcgagaaggagaggaagtttaaccctgatctggccccaggcaccgagaaggtgac
aagagagggccagaagggcgagaagaccatcaccacacccacactgaagaatcctctgaccggcgagatcatcagcaagggcgagtccaag
gaggagatcacaaaggaccccatcaacgaactgaccgaatggggaccagagacaggaggaagcggcagcggcggaagcagc
IC1/IC2
GGSGSGSG (SEQ ID NO: 50)
Ggaggcagcggcagcggcagcggg (SEQ ID NO: 48)
Membrane bound domains
PDGFR
NAVGQDTQEVIVVPHSLPFKVVVISAILALVVLTIISLIILIMLWQKKPR (SEQ ID NO: 232)
Aacgccgtgggccaggacacccaggaagtgatcgtggtgccccacagcctgcctttcaaggtggtggtcatctccgccatcctggccctggtcgt
gctgactattatttccctgattatcctgattatgctgtggcagaagaagcccaga (SEQ ID NO: 230)
Foldon
YIPEAPRDGQAYVRKDGEWVLLSTFL (SEQ ID NO: 235)
Tacatccctgaggccccaagggacggacaggcctatgtgagaaaggatggcgagtgggtgctgctgtccaccttcctg (SEQ ID
NO: 233)
Nanoparticle domains
3BVE (amino acid, dna, rna)
GLSKDIIKLLNEQVNKEMQSSNLYMSMSSWCYTHSLDGAGLFLFDHAAEEYEHAKKLIIFLNENNVPVQLTSISAPE
HKFEGLTQIFQKAYEHEQHISESINNIVDHAIKSKDHATFNFLQWYVAEQHEEEVLFKDILDKIELIGNENHGLYLAD
QYVKGIAKSRKS (SEQ ID NO: 135)
Gggctgagtaaggacattatcaagctgctgaacgaacaggtgaacaaagagatgcagtctagcaacctgtacatgtccatgagctcctggtgct
atacccactctctggacggagcaggcctgttcctgtttgatcacgccgccgaggagtacgagcacgccaagaagctgatcatcttcctgaatgag
aacaatgtgcccgtgcagctgacctctatcagcgcccctgagcacaagttcgagggcctgacacagatctttcagaaggcctacgagcacgagc
agcacatctccgagtctatcaacaatatcgtggaccacgccatcaagtccaaggatcacgccacattcaactttctgcagtggtacgtggccgag
cagcacgaggaggaggtgctgtttaaggacatcctggataagatcgagctgatcggcaatgagaaccacgggctgtacctggcagatcagtat
gtcaagggcatcgctaagtcaaggaaaagc (SEQ ID NO: 133)
GGGCUGAGUAAGGACAUUAUCAAGCUGCUGAACGAACAGGUGAACAAAGAGAUGCAGUCUAGCAACCU
GUACAUGUCCAUGAGCUCCUGGUGCUAUACCCACUCUCUGGACGGAGCAGGCCUGUUCCUGUUUGAUC
ACGCCGCCGAGGAGUACGAGCACGCCAAGAAGCUGAUCAUCUUCCUGAAUGAGAACAAUGUGCCCGUGC
AGCUGACCUCUAUCAGCGCCCCUGAGCACAAGUUCGAGGGCCUGACACAGAUCUUUCAGAAGGCCUACG
AGCACGAGCAGCACAUCUCCGAGUCUAUCAACAAUAUCGUGGACCACGCCAUCAAGUCCAAGGAUCACGC
CACAUUCAACUUUCUGCAGUGGUACGUGGCCGAGCAGCACGAGGAGGAGGUGCUGUUUAAGGACAUCC
UGGAUAAGAUCGAGCUGAUCGGCAAUGAGAACCACGGGCUGUACCUGGCAGAUCAGUAUGUCAAGGGC
AUCGCUAAGUCAAGGAAAAGC (SEQ ID NO: 134)
I3 (amino acid, dna, rna)
MKMEELFKKHKIVAVLRANSVEEAKKKALAVFLGGVHLIEITFTVPDADTVIKELSFLKEMGAIIGAGTVTSVEQCRK
AVESGAEFIVSPHLDEEISQFCKEKGVFYMPGVMTPTELVKAMKLGHTILKLFPGEVVGPQFVKAMKGPFPNVKFV
PTGGVNLDNVCEWFKAGVLAVGVGSALVKGTPVEVAEKAKAFVEKIRGCTE (SEQ ID NO: 138)
Atgaagatggaagaactgttcaagaagcacaagatcgtggccgtgctgagggccaactccgtggaggaggccaagaagaaggccctggccgt
gttcctgggcggcgtgcacctgatcgagatcacctttacagtgcccgacgccgataccgtgatcaaggagctgtctttcctgaaggagatgggag
caatcatcggagcaggaaccgtgacaagcgtggagcagtgcagaaaggccgtggagagcggcgccgagtttatcgtgtcccctcacctggacg
aggagatctctcagttctgtaaggagaagggcgtgttttacatgccaggcgtgatgacccccacagagctggtgaaggccatgaagctgggcca
cacaatcctgaagctgttccctggcgaggtggtgggcccacagtttgtgaaggccatgaagggccccttccctaatgtgaagtttgtgcccaccg
gcggcgtgaacctggataacgtgtgcgagtggttcaaggcaggcgtgctggcagtgggcgtgggcagcgccctggtgaagggcacacccgtgg
aagtcgctgagaaggcaaaggcattcgtggaaaagattagggggtgtactgag (SEQ ID NO: 136)
AUGAAGAUGGAAGAACUGUUCAAGAAGCACAAGAUCGUGGCCGUGCUGAGGGCCAACUCCGUGGAGGA
GGCCAAGAAGAAGGCCCUGGCCGUGUUCCUGGGCGGCGUGCACCUGAUCGAGAUCACCUUUACAGUGCC
CGACGCCGAUACCGUGAUCAAGGAGCUGUCUUUCCUGAAGGAGAUGGGAGCAAUCAUCGGAGCAGGAA
CCGUGACAAGCGUGGAGCAGUGCAGAAAGGCCGUGGAGAGCGGCGCCGAGUUUAUCGUGUCCCCUCACC
UGGACGAGGAGAUCUCUCAGUUCUGUAAGGAGAAGGGCGUGUUUUACAUGCCAGGCGUGAUGACCCCC
ACAGAGCUGGUGAAGGCCAUGAAGCUGGGCCACACAAUCCUGAAGCUGUUCCCUGGCGAGGUGGUGGG
CCCACAGUUUGUGAAGGCCAUGAAGGGCCCCUUCCCUAAUGUGAAGUUUGUGCCCACCGGCGGCGUGAA
CCUGGAUAACGUGUGCGAGUGGUUCAAGGCAGGCGUGCUGGCAGUGGGCGUGGGCAGCGCCCUGGUG
AAGGGCACACCCGUGGAAGUCGCUGAGAAGGCAAAGGCAUUCGUGGAAAAGAUUAGGGGGUGUACUGA
G (SEQ ID NO: 137)
LS (amino acid, dna, rna)

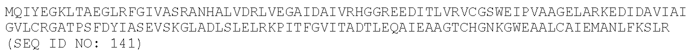

Atgcagatctacgaaggaaaactgaccgctgagggactgaggttcggaattgtcgcaagccgcgcgaatcacgcactggtggataggctggtg
gaaggcgctatcgacgcaattgtccggcacggcgggagagaggaagacatcacactggtgagagtctgcggcagctgggagattcccgtggca
gctggagaactggctcgaaaggaggacatcgatgccgtgatcgctattggggtcctgtgccgaggagcaactcccagcttcgactacatcgcctc
agaagtgagcaaggggctggctgatctgtccctggagctgaggaaacctatcacttttggcgtgattactgccgacaccctggaacaggcaatc
gaggcggccggcacctgccatggaaacaaaggctgggaagcagccctgtgcgctattgagatggcaaatctgttcaaatctctgcga (SEQ
ID NO: 139)

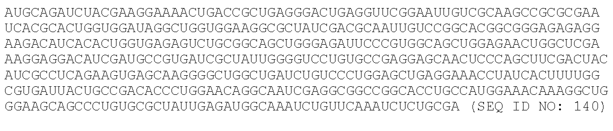

QB (amino acid, dna, rna)
AKLETVTLGNIGKDGKQTLVLNPRGVNPTNGVASLSQAGAVPALEKRVTVSVSQPSRNRKNYKVQVKIQNPTACT
ANGSCDPSVTRQAYADVTFSFTQYSTDEERAFVRTELAALLASPLLIDAIDQLNPAY (SEQ ID NO: 144)
Gcaaagctggagacagtgacactgggcaacatcggcaaggacggcaagcagacactggtgctgaatcccaggggcgtgaaccctaccaatg
gagtggcatctctgagccaggcaggagcagtgcctgccctggagaagagagtgaccgtgtccgtgtctcagcccagcaggaacagaaagaatt
ataaggtgcaggtgaagatccagaacccaaccgcctgcacagccaatggcagctgtgacccatccgtgacaaggcaggcatacgcagatgtga
ccttctcttttacacagtatagcaccgatgaggagagggccttcgtgcgcaccgagctggccgccctgctggcatcccctctgctgattgacg
ctattgaccagctgaaccctgcttac (SEQ ID NO: 142)
GCAAAGCUGGAGACAGUGACACUGGGCAACAUCGGCAAGGACGGCAAGCAGACACUGGUGCUGAAUCCC
AGGGGCGUGAACCCUACCAAUGGAGUGGCAUCUCUGAGCCAGGCAGGAGCAGUGCCUGCCCUGGAGAA
GAGAGUGACCGUGUCCGUGUCUCAGCCCAGCAGGAACAGAAAGAAUUAUAAGGUGCAGGUGAAGAUCC
AGAACCCAACCGCCUGCACAGCCAAUGGCAGCUGUGACCCAUCCGUGACAAGGCAGGCAUACGCAGAUGU
GACCUUCUCUUUUACACAGUAUAGCACCGAUGAGGAGAGGGCCUUCGUGCGCACCGAGCUGGCCGCCCU
GCUGGCAUCCCCUCUGCUGAUUGACGCUAUUGACCAGCUGAACCCUGCUUAC (SEQ ID NO: 143)
IC1 (amino acid, dna, rna)
DPEFTKNALNVVKNDLIAKVDQLSGEQEVLRGELEAAKQAKVKLENRIKELEEELKRV (SEQ ID NO: 147)
Gaccctgagtttaccaaaaatgctctgaatgtcgtcaaaaatgatctgattgctaaggtggaccagctgagcggagagcaggaggtgctgagg
ggcgagctggaggccgccaagcaggcaaaggtgaaactggaaaaccgaatcaaggaactggaagaagaactgaaaagagtc (SEQ ID
NO: 145)
GACCCUGAGUUUACCAAAAAUGCUCUGAAUGUCGUCAAAAAUGAUCUGAUUGCUAAGGUGGACCAGCU
GAGCGGAGAGCAGGAGGUGCUGAGGGGCGAGCUGGAGGCCGCCAAGCAGGCAAAGGUGAAACUGGAAA
ACCGAAUCAAGGAACUGGAAGAAGAACUGAAAAGAGUC (SEQ ID NO: 146)
IC2 (amino acid, dna, rna)
ADPKKVLDKAKDQAENRVRELKQKLEELYKEARKLDLTQEMRRKLELRYIAAMLMAIGDIYNAIRQAKQEADKLKK
AGLVNSQQLDELKRRLEELKEEASRKARDYGREFQLKLEYGGGSGSGSGGKIEQILQKIEKILQKIEWILQKIEQILQG
(SEQ ID NO: 150)
Gccgaccccaagaaggtgctggataaagccaaagatcaggcagaaaatagagtcagggaactgaagcagaagctggaggagctgtacaag
gaggcccggaagctggacctgacccaggagatgaggagaaagctggagctgcgctacatcgccgccatgctgatggccatcggcgacatctat
aacgccatcaggcaggccaagcaggaggccgataagctgaagaaggccggcctggtgaatagccagcagctggacgagctgaagcggcgcc
tggaggagctgaaggaggaggccagcaggaaggccagagattacggcagggagttccagctgaagctggagtatggcggcggcagcggctc
cggctctggcggcaagatcgagcagatcctgcagaagatcgaaaagatcctgcagaagattgagtggattctgcagaagattgaacagatcct
gcagggg (SEQ ID NO: 148)
GCCGACCCCAAGAAGGUGCUGGAUAAAGCCAAAGAUCAGGCAGAAAAUAGAGUCAGGGAACUGAAGCAG
AAGCUGGAGGAGCUGUACAAGGAGGCCCGGAAGCUGGACCUGACCCAGGAGAUGAGGAGAAAGCUGGA
GCUGCGCUACAUCGCCGCCAUGCUGAUGGCCAUCGGCGACAUCUAUAACGCCAUCAGGCAGGCCAAGCA
GGAGGCCGAUAAGCUGAAGAAGGCCGGCCUGGUGAAUAGCCAGCAGCUGGACGAGCUGAAGCGGCGCC
UGGAGGAGCUGAAGGAGGAGGCCAGCAGGAAGGCCAGAGAUUACGGCAGGGAGUUCCAGCUGAAGCUG
GAGUAUGGCGGCGGCAGCGGCUCCGGCUCUGGCGGCAAGAUCGAGCAGAUCCUGCAGAAGAUCGAAAA
GAUCCUGCAGAAGAUUGAGUGGAUUCUGCAGAAGAUUGAACAGAUCCUGCAGGGG (SEQ ID NO: 149)
Env sections
MD39 gp120 (amino acid, dna, rna)
same for MD39, GRSF, link14, Trimer strings 1, Trimer strings 2, MD39_link14_PDGFR,
MD39_link14_gp140_Foldon_PDGFR
AENLWVTVYYGVPVWKDAETTLFCASDAKAYETEKHNVWATHACVPTDPNPQEIHLENVTEEFNMWKNNMVE
QMHEDIISLWDQSLKPCVKLTPLCVTLQCTNVTNNITDDMRGELKNCSFNMTTELRDKKQKVYSLFYRLDVVQINE
NQGNRSNNSNKEYRLINCNTSAITQACPKVSFEPIPIHYCAPAGFAILKCKDKKFNGTGPCPSVSTVQCTHGIKPVVS
TQLLLNGSLAEEEVIIRSENITNNAKNILVQLNTPVQINCTRPNNNTVKSIRIGPGQAFYYTGDIIGDIRQAHCNVSKA
TWNETLGKVVKQLRKHFGNNTIIRFAQSSGGDLEVTTHSFNCGGEFFYCNTSGLFNSTWISNTSVQGSNSTGSNDS
ITLPCRIKQIINMWQRIGQAMYAPPIQGVIRCVSNITGLILTRDGGSTNSTTETFRPGGGDMRDNWRSELYKYKVV
KIEPLGVAPTRCKRRVVG (SEQ ID NO: 55)
gccgaaaacctgtgggtcaccgtctactatggagtgcccgtgtggaaggacgccgagactacgctgttctgcgccagcgatgccaaggcctacg
agacagagaagcacaacgtgtgggcaacccacgcatgcgtgcctacagacccaaacccccaggagatccacctggagaatgtgacagaggag
tttaacatgtggaagaacaatatggtggagcagatgcacgaggacatcatctccctgtgggatcagtctctgaagccctgcgtgaagctgacccc
tctgtgcgtgacactgcagtgtaccaacgtgacaaacaatatcaccgacgatatgcggggcgagctgaagaattgtagcttcaacatgaccaca
gagctgagggacaagaagcagaaggtgtactccctgttttatagactggatgtggtgcagatcaatgagaaccagggcaatcggtctaacaata
gcaacaaggagtaccgcctgatcaattgcaacacctccgccatcacacaggcctgtcctaaggtgtctttcgagcctatcccaatccactattgcg
ccccagccggcttcgccatcctgaagtgtaaggataagaagtttaacggaaccggaccatgcccttccgtgtctaccgtgcagtgtacacacggc
atcaagcctgtggtgtctacacagctgctgctgaatggcagcctggccgaggaggaagtgatcatcaggtctgagaacatcaccaacaatgcca
agaatatcctggtgcagctgaacacaccagtgcagatcaattgcacccggcccaacaataacacagtgaagtctatccgcatcggcccaggcca
ggccttttactataccggcgacatcatcggcgatatcagacaggcccactgtaatgtgagcaaggccacctggaacgagacactgggcaaggtg
gtgaagcagctgaggaagcacttcggcaataacaccatcatcagatttgcacagagctccggcggcgacctggaggtgaccacacactccttca
attgcggcggcgagttcttttactgtaacacaagcggcctgtttaattccacctggatctccaacacatctgtgcagggcagcaattccaccggca
gcaacgattccatcacactgccatgccggatcaagcagatcatcaacatgtggcagcgcatcggccaggccatgtatgcccctcccatccaggg
cgtgatcagatgcgtgagcaatatcaccggcctgatcctgacacgcgacggcggctctaccaacagcaccacagagacattccggcccggcgg
cggcgacatgagggataactggagatctgagctgtacaagtataaggtggtgaagatcgagcctctgggagtggcaccaaccaggtgcaagag
gagagtggtgggc (SEQ ID NO: 53)
GCCGAAAACCUGUGGGUCACCGUCUACUAUGGAGUGCCCGUGUGGAAGGACGCCGAGACUACGCUGUU
CUGCGCCAGCGAUGCCAAGGCCUACGAGACAGAGAAGCACAACGUGUGGGCAACCCACGCAUGCGUGCC
UACAGACCCAAACCCCCAGGAGAUCCACCUGGAGAAUGUGACAGAGGAGUUUAACAUGUGGAAGAACAA
UAUGGUGGAGCAGAUGCACGAGGACAUCAUCUCCCUGUGGGAUCAGUCUCUGAAGCCCUGCGUGAAGC
UGACCCCUCUGUGCGUGACACUGCAGUGUACCAACGUGACAAACAAUAUCACCGACGAUAUGCGGGGCG
AGCUGAAGAAUUGUAGCUUCAACAUGACCACAGAGCUGAGGGACAAGAAGCAGAAGGUGUACUCCCUG
UUUUAUAGACUGGAUGUGGUGCAGAUCAAUGAGAACCAGGGCAAUCGGUCUAACAAUAGCAACAAGGA
GUACCGCCUGAUCAAUUGCAACACCUCCGCCAUCACACAGGCCUGUCCUAAGGUGUCUUUCGAGCCUAU
CCCAAUCCACUAUUGCGCCCCAGCCGGCUUCGCCAUCCUGAAGUGUAAGGAUAAGAAGUUUAACGGAAC
CGGACCAUGCCCUUCCGUGUCUACCGUGCAGUGUACACACGGCAUCAAGCCUGUGGUGUCUACACAGCU
GCUGCUGAAUGGCAGCCUGGCCGAGGAGGAAGUGAUCAUCAGGUCUGAGAACAUCACCAACAAUGCCAA
GAAUAUCCUGGUGCAGCUGAACACACCAGUGCAGAUCAAUUGCACCCGGCCCAACAAUAACACAGUGAA
GUCUAUCCGCAUCGGCCCAGGCCAGGCCUUUUACUAUACCGGCGACAUCAUCGGCGAUAUCAGACAGGC
CCACUGUAAUGUGAGCAAGGCCACCUGGAACGAGACACUGGGCAAGGUGGUGAAGCAGCUGAGGAAGC
ACUUCGGCAAUAACACCAUCAUCAGAUUUGCACAGAGCUCCGGCGGCGACCUGGAGGUGACCACACACU
CCUUCAAUUGCGGCGGCGAGUUCUUUUACUGUAACACAAGCGGCCUGUUUAAUUCCACCUGGAUCUCC
AACACAUCUGUGCAGGGCAGCAAUUCCACCGGCAGCAACGAUUCCAUCACACUGCCAUGCCGGAUCAAGC
AGAUCAUCAACAUGUGGCAGCGCAUCGGCCAGGCCAUGUAUGCCCCUCCCAUCCAGGGCGUGAUCAGAU
GCGUGAGCAAUAUCACCGGCCUGAUCCUGACACGCGACGGCGGCUCUACCAACAGCACCACAGAGACAUU
CCGGCCCGGCGGCGGCGACAUGAGGGAUAACUGGAGAUCUGAGCUGUACAAGUAUAAGGUGGUGAAGA
UCGAGCCUCUGGGAGUGGCACCAACCAGGUGCAAGAGGAGAGUGGUGGGC (SEQ ID NO: 54)
gp120 for CPG9.2 (amino acid, dna, rna)
LWVTVYYGVPVWKDAETTLFCASDAKAYETEKHNVWATHACVPTDPNPQEIHLENVTEEFNMWKNNMVEQM
HEDIISLWDQSLKPCVKLTPLCVTLQCTNVTNNITDDMRGELKNCSFNMTTELRDKKQKVYSLFYRLDVVQINENQ
GNRSNNSNKEYRLINCNTSAITQACPKVSFEPIPIHYCAPAGFAILKCKDKKFNGTGPCPSVSTVQCTHGIKPVVSTQ
LLLNGSLAEEEVIIRSENITNNAKNILVQLNTPVQINCTRPNNNTVKSIRIGPGQAFYYTGDIIGDIRQAHCNVSKATW
NETLGKVVKQLRKHFGNNTIIRFAQSSGGDLEVTTHSFNCGGEFFYCNTSGLFNSTWISNTSVQGSNSTGSNDSITL
PCRIKQIINMWQRIGQAMYAPPIQGVIRCVSNITGLILTRDGGSTNSTTETFRPGGGDMRDNWRSELYKYKVVKIE
PLGVAPTRCNRS (SEQ ID NO: 58)
ctgtgggtgaccgtgtactatggcgtgcccgtgtggaaggacgccgagactacgctgttctgcgcctccgatgccaaggcctatgagacagaga
agcacaacgtgtgggcaacccacgcatgcgtgccaacagaccctaacccacaggagatccacctggagaatgtgaccgaggagtttaacatgt
ggaagaacaatatggtggagcagatgcacgaggacatcatcagcctgtgggatcagtccctgaagccttgcgtgaagctgaccccactgtgcgt
gacactgcagtgtaccaacgtgacaaacaatatcaccgacgatatgaggggcgagctgaagaattgttctttcaacatgaccacagagctgag
ggacaagaagcagaaagtgtacagcctgttttatagactggatgtggtgcagatcaatgagaaccagggcaataggagcaacaattccaacaa
ggagtacagactgatcaattgcaacaccagcgccatcacacaggcctgtccaaaggtgtccttcgagcccatccctatccactattgcgcaccag
caggattcgcaatcctgaagtgtaaggataagaagtttaacggaaccggaccatgcccatctgtgagcaccgtgcagtgtacacacggcatcaa
gccagtggtgtccacacagctgctgctgaatggctctctggccgaggaggaagtgatcatccggagcgagaacatcaccaacaatgccaagaa
tatcctggtgcagctgaacacacccgtgcagatcaattgcacccggcctaacaataacacagtgaagtccatcaggatcggaccaggacaggc
cttttactataccggcgacatcatcggcgatatccgccaggcccactgtaacgtgagcaaggccacctggaacgagacactgggcaaggtggtg
aagcagctgaggaagcacttcggcaataacaccatcatcagatttgcacagtcctctggcggcgacctggaggtgaccacacactccttcaact
gcggcggcgagttcttttactgtaacacatctggcctgtttaatagcacctggatctctaacacaagcgtgcagggctccaattctaccggctcca
acgattctatcacactgccctgccggatcaagcagatcatcaacatgtggcagaggatcggacaggcaatgtacgcccctcccatccagggcgt
gatcagatgcgtgagcaatatcaccggcctgatcctgacacgcgacggcggcagcaccaactccaccacagagacattcagacccggcggcgg
cgacatgagggataactggagatccgagctgtataagtataaagtcgtgaagattgagccactgggcgtcgcaccaacaagatgtaatagaag
c (SEQ ID NO: 56)
CUGUGGGUGACCGUGUACUAUGGCGUGCCCGUGUGGAAGGACGCCGAGACUACGCUGUUCUGCGCCUC
CGAUGCCAAGGCCUAUGAGACAGAGAAGCACAACGUGUGGGCAACCCACGCAUGCGUGCCAACAGACCCU
AACCCACAGGAGAUCCACCUGGAGAAUGUGACCGAGGAGUUUAACAUGUGGAAGAACAAUAUGGUGGA
GCAGAUGCACGAGGACAUCAUCAGCCUGUGGGAUCAGUCCCUGAAGCCUUGCGUGAAGCUGACCCCACU
GUGCGUGACACUGCAGUGUACCAACGUGACAAACAAUAUCACCGACGAUAUGAGGGGCGAGCUGAAGAA
UUGUUCUUUCAACAUGACCACAGAGCUGAGGGACAAGAAGCAGAAAGUGUACAGCCUGUUUUAUAGAC
UGGAUGUGGUGCAGAUCAAUGAGAACCAGGGCAAUAGGAGCAACAAUUCCAACAAGGAGUACAGACUG
AUCAAUUGCAACACCAGCGCCAUCACACAGGCCUGUCCAAAGGUGUCCUUCGAGCCCAUCCCUAUCCACU
AUUGCGCACCAGCAGGAUUCGCAAUCCUGAAGUGUAAGGAUAAGAAGUUUAACGGAACCGGACCAUGCC
CAUCUGUGAGCACCGUGCAGUGUACACACGGCAUCAAGCCAGUGGUGUCCACACAGCUGCUGCUGAAUG
GCUCUCUGGCCGAGGAGGAAGUGAUCAUCCGGAGCGAGAACAUCACCAACAAUGCCAAGAAUAUCCUGG
UGCAGCUGAACACACCCGUGCAGAUCAAUUGCACCCGGCCUAACAAUAACACAGUGAAGUCCAUCAGGAU
CGGACCAGGACAGGCCUUUUACUAUACCGGCGACAUCAUCGGCGAUAUCCGCCAGGCCCACUGUAACGU
GAGCAAGGCCACCUGGAACGAGACACUGGGCAAGGUGGUGAAGCAGCUGAGGAAGCACUUCGGCAAUAA
CACCAUCAUCAGAUUUGCACAGUCCUCUGGCGGCGACCUGGAGGUGACCACACACUCCUUCAACUGCGG
CGGCGAGUUCUUUUACUGUAACACAUCUGGCCUGUUUAAUAGCACCUGGAUCUCUAACACAAGCGUGC
AGGGCUCCAAUUCUACCGGCUCCAACGAUUCUAUCACACUGCCCUGCCGGAUCAAGCAGAUCAUCAACAU
GUGGCAGAGGAUCGGACAGGCAAUGUACGCCCCUCCCAUCCAGGGCGUGAUCAGAUGCGUGAGCAAUAU
CACCGGCCUGAUCCUGACACGCGACGGCGGCAGCACCAACUCCACCACAGAGACAUUCAGACCCGGCGGC
GGCGACAUGAGGGAUAACUGGAGAUCCGAGCUGUAUAAGUAUAAAGUCGUGAAGAUUGAGCCACUGGG
CGUCGCACCAACAAGAUGUAAUAGAAGC (SEQ ID NO: 57)
MD39 gp41 ecto (amino acid, dna, rna)
same for BG505 MD39 link 14, MD39_PDGFR
AVGIGAVSLGFLGAAGSTMGAASMTLTVQARNLLSGIVQQQSNLLRAPEPQQHLLKDTHWGIKQLQARVLAVEH
YLRDQQLLGIWGCSGKLICCTNVPWNSSWSNRNLSEIWDNMTWLQWDKEISNYTQIIYGLLEESQNQQEKNEQD
LLALD (SEQ ID NO: 80)
Gcagtgggcatcggagccgtgtccctgggctttctgggagcagcaggctccacaatgggagcagcctctatgaccctgacagtgcaggccagg
aatctgctgagcggcatcgtgcagcagcagtccaacctgctgagagccccagagccccagcagcacctgctgaaggacacccactggggcatc
aagcagctgcaggccagggtgctggcagtggagcactatctgagagatcagcagctgctgggcatctggggctgtagcggcaagctgatctgct
gtaccaatgtgccctggaactctagctggtctaatcgcaacctgagcgagatctgggacaatatgacctggctgcagtgggataaggagatctcc
aactacacacagatcatctatggcctgctggaagaatctcagaatcagcaggaaaagaatgaacaggatctgctggcactggat (SEQ ID
NO: 78)
GCAGUGGGCAUCGGAGCCGUGUCCCUGGGCUUUCUGGGAGCAGCAGGCUCCACAAUGGGAGCAGCCUC
UAUGACCCUGACAGUGCAGGCCAGGAAUCUGCUGAGCGGCAUCGUGCAGCAGCAGUCCAACCUGCUGAG
AGCCCCAGAGCCCCAGCAGCACCUGCUGAAGGACACCCACUGGGGCAUCAAGCAGCUGCAGGCCAGGGUG
CUGGCAGUGGAGCACUAUCUGAGAGAUCAGCAGCUGCUGGGCAUCUGGGGCUGUAGCGGCAAGCUGAU
CUGCUGUACCAAUGUGCCCUGGAACUCUAGCUGGUCUAAUCGCAACCUGAGCGAGAUCUGGGACAAUA
UGACCUGGCUGCAGUGGGAUAAGGAGAUCUCCAACUACACACAGAUCAUCUAUGGCCUGCUGGAAGAA
UCUCAGAAUCAGCAGGAAAAGAAUGAACAGGAUCUGCUGGCACUGGAU (SEQ ID NO: 79)
BG505_MD39 GRSF gp41 ecto (amino acid, dna, rna)
glycan sites added (underline)
AVGIGAVSLGFLGAAGSTMGAASMTLTVQARNLLSGIVQQQSNLLRAPEPQQHLLKDTHWGIKQLQARVLAVEH
YLRDQQLLGIWGCSGKLICCTNVPWNSSWSNRNLSEIWDNMTWLNWSKEISNYTQIIYGLLEESQNQQEKNNQS
LLALD (SEQ ID NO: 83)
Gcagtgggcatcggagccgtgtccctgggctttctgggagcagcaggctccacaatgggagcagcctctatgaccctgacagtgcaggccagg
aatctgctgagcggcatcgtgcagcagcagtccaacctgctgagagccccagagccccagcagcacctgctgaaggacacccactggggcatc
aagcagctgcaggccagggtgctggcagtggagcactatctgagagatcagcagctgctgggcatctggggctgtagcggcaagctgatctgct
gtaccaatgtgccctggaactctagctggtctaatcgcaacctgagcgagatctgggacaatatgacctggctgaactggagcaaggagatctc
caactacacacagatcatctatggcctgctggaagaatctcagaatcagcaggaaaagaataaccagagcctgctggcactggat (SEQ ID
NO: 81)
GCAGUGGGCAUCGGAGCCGUGUCCCUGGGCUUUCUGGGAGCAGCAGGCUCCACAAUGGGAGCAGCCUC
UAUGACCCUGACAGUGCAGGCCAGGAAUCUGCUGAGCGGCAUCGUGCAGCAGCAGUCCAACCUGCUGAG
AGCCCCAGAGCCCCAGCAGCACCUGCUGAAGGACACCCACUGGGGCAUCAAGCAGCUGCAGGCCAGGGUG
CUGGCAGUGGAGCACUAUCUGAGAGAUCAGCAGCUGCUGGGCAUCUGGGGCUGUAGCGGCAAGCUGAU
CUGCUGUACCAAUGUGCCCUGGAACUCUAGCUGGUCUAAUCGCAACCUGAGCGAGAUCUGGGACAAUA
UGACCUGGCUGAACUGGAGCAAGGAGAUCUCCAACUACACACAGAUCAUCUAUGGCCUGCUGGAAGAAU
CUCAGAAUCAGCAGGAAAAGAAUAACCAGAGCCUGCUGGCACUGGAU (SEQ ID NO: 82)
BG505_MD39 CPG9.2 gp41 ecto (amino acid, dna, rna)
SLGFLGAAGSTMGAASMTLTVQARNLLSGIVQQQSNLLRAPEPQQHLLKDTHWGIKQLQARVLAVEHYLRDQQL
LGIWGCSGKLICCTNVPWNSSWSNRNLSEIWDNMTWLNWSKEISNYTQIIYGLLEESQNQNESNEQDL (SEQ ID
NO: 86)
Agcctggggttcctgggagcagcaggctccaccatgggagcagcatctatgaccctgacagtgcaggccaggaatctgctgtctggcatcgtgc
agcagcagagcaacctgctgagagccccagagccccagcagcacctgctgaaggacacccactggggcatcaagcagctgcaggcccgggtg
ctggcagtggagcactacctgcgcgatcagcagctgctgggaatctggggatgcagcggcaagctgatctgctgtacaaatgtgccttggaaca
gctcctggtccaataggaacctgtctgagatctgggacaatatgacctggctgaactggtctaaggagatcagcaattacacacagatcatctat
ggcctgctggaggagagccagaatcagaacgagtccaatgagcaggatctg (SEQ ID NO: 84)
AGCCUGGGGUUCCUGGGAGCAGCAGGCUCCACCAUGGGAGCAGCAUCUAUGACCCUGACAGUGCAGGCC
AGGAAUCUGCUGUCUGGCAUCGUGCAGCAGCAGAGCAACCUGCUGAGAGCCCCAGAGCCCCAGCAGCACC
UGCUGAAGGACACCCACUGGGGCAUCAAGCAGCUGCAGGCCCGGGUGCUGGCAGUGGAGCACUACCUGC
GCGAUCAGCAGCUGCUGGGAAUCUGGGGAUGCAGCGGCAAGCUGAUCUGCUGUACAAAUGUGCCUUGG
AACAGCUCCUGGUCCAAUAGGAACCUGUCUGAGAUCUGGGACAAUAUGACCUGGCUGAACUGGUCUAA
GGAGAUCAGCAAUUACACACAGAUCAUCUAUGGCCUGCUGGAGGAGAGCCAGAAUCAGAACGAGUCCAA
UGAGCAGGAUCUG (SEQ ID NO: 85)
BG505 full length sequences
Soluble
BG505 MD39 (amino acid, dna, rna)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGRRRRRRAVGIGAVSLGFLGAAGSTMGAASMTLTVQAR
NLLSGIVQQQSNLLRAPEPQQHLLKDTHWGIKQLQARVLAVEHYLRDQQLLGIWGCSGKLICCTNVPWNSSWSN
RNLSEIWDNMTWLQWDKEISNYTQIIYGLLEESQNQQEKNEQDLLALD** (SEQ ID NO: 108)
atggactggacatggattctgttcctggtcgctgccgctacaagagtgcattccgccgaaaacctgtgggtcaccgtctactatggagtgcccgtg
tggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccg
ccatcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggca
gcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggccggcgcaggagacggcgcgcagtg
ggcatcggagccgtgtccctgggctttctgggagcagcaggctccacaatgggagcagcctctatgaccctgacagtgcaggccaggaatctgc
tgagcggcatcgtgcagcagcagtccaacctgctgagagccccagagccccagcagcacctgctgaaggacacccactggggcatcaagcagc
tgcaggccagggtgctggcagtggagcactatctgagagatcagcagctgctgggcatctggggctgtagcggcaagctgatctgctgtaccaa
tgtgccctggaactctagctggtctaatcgcaacctgagcgagatctgggacaatatgacctggctgcagtgggataaggagatctccaactaca
cacagatcatctatggcctgctggaagaatctcagaatcagcaggaaaagaatgaacaggatctgctggcactggattgataa (SEQ ID
NO: 106)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCCGGCGCAGGAGACGGCGCGCAGUGGGCAUCGGAGCC
GUGUCCCUGGGCUUUCUGGGAGCAGCAGGCUCCACAAUGGGAGCAGCCUCUAUGACCCUGACAGUGCA
GGCCAGGAAUCUGCUGAGCGGCAUCGUGCAGCAGCAGUCCAACCUGCUGAGAGCCCCAGAGCCCCAGCA
GCACCUGCUGAAGGACACCCACUGGGGCAUCAAGCAGCUGCAGGCCAGGGUGCUGGCAGUGGAGCACUA
UCUGAGAGAUCAGCAGCUGCUGGGCAUCUGGGGCUGUAGCGGCAAGCUGAUCUGCUGUACCAAUGUGC
CCUGGAACUCUAGCUGGUCUAAUCGCAACCUGAGCGAGAUCUGGGACAAUAUGACCUGGCUGCAGUGG
GAUAAGGAGAUCUCCAACUACACACAGAUCAUCUAUGGCCUGCUGGAAGAAUCUCAGAAUCAGCAGGAA
AAGAAUGAACAGGAUCUGCUGGCACUGGAUUGAUAA (SEQ ID NO: 105)
BG505 MD39 GRSF (amino acid, dna, rna)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKWKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGRRRRRRAVGIGAVSLGFLGAAGSTMGAASMTLTVQAR
NLLSGIVQQQSNLLRAPEPQQHLLKDTHWGIKQLQARVLAVEHYLRDQQLLGIWGCSGKLICCTNVPWNSSWSN
RNLSEIWDNMTWLNWSKEISNYTQIIYGLLEESQNQQEKNNQSLLALD** (SEQ ID NO: 111)
(bold underline are mutations for glycosylation sites added)
atggactggacatggattctgttcctggtcgctgccgctacaagagtgcattccgccgaaaacctgtgggtcaccgtctactatggagtgcccgtg
tggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccg
ccatcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggca
gcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggccggcgcaggagacggcgcgcagtg
ggcatcggagccgtgtccctgggctttctgggagcagcaggctccacaatgggagcagcctctatgaccctgacagtgcaggccaggaatctgc
tgagcggcatcgtgcagcagcagtccaacctgctgagagccccagagccccagcagcacctgctgaaggacacccactggggcatcaagcagc
tgcaggccagggtgctggcagtggagcactatctgagagatcagcagctgctgggcatctggggctgtagcggcaagctgatctgctgtaccaa
tgtgccctggaactctagctggtctaatcgcaacctgagcgagatctgggacaatatgacctggctgaactggagcaaggagatctccaactac
acacagatcatctatggcctgctggaagaatctcagaatcagcaggaaaagaataaccagagcctgctggcactggattgataa (SEQ ID
NO: 109)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCCGGCGCAGGAGACGGCGCGCAGUGGGCAUCGGAGCC
GUGUCCCUGGGCUUUCUGGGAGCAGCAGGCUCCACAAUGGGAGCAGCCUCUAUGACCCUGACAGUGCA
GGCCAGGAAUCUGCUGAGCGGCAUCGUGCAGCAGCAGUCCAACCUGCUGAGAGCCCCAGAGCCCCAGCA
GCACCUGCUGAAGGACACCCACUGGGGCAUCAAGCAGCUGCAGGCCAGGGUGCUGGCAGUGGAGCACUA
UCUGAGAGAUCAGCAGCUGCUGGGCAUCUGGGGCUGUAGCGGCAAGCUGAUCUGCUGUACCAAUGUGC
CCUGGAACUCUAGCUGGUCUAAUCGCAACCUGAGCGAGAUCUGGGACAAUAUGACCUGGCUGAACUGG
AGCAAGGAGAUCUCCAACUACACACAGAUCAUCUAUGGCCUGCUGGAAGAAUCUCAGAAUCAGCAGGAA
AAGAAUAACCAGAGCCUGCUGGCACUGGAUUGAUAA (SEQ ID NO: 110)
BG505 MD39 Link 14 (amino acid, dna, rna)
(cleavage independent)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGSHSGSGGSGSGGHAAVGIGAVSLGFLGAAGSTMGAAS
MTLTVQARNLLSGIVQQQSNLLRAPEPQQHLLKDTHWGIKQLQARVLAVEHYLRDQQLLGIWGCSGKLICCTNVP
WNSSWSNRNLSEIWDNMTWLQWDKEISNYTQIIYGLLEESQNQQEKNEQDLLALD** (SEQ ID NO: 114)
atggactggacatggattctgttcctggtcgctgccgctacaagagtgcattccgccgaaaacctgtgggtcaccgtctactatggagtgcccgtg
tggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccg
ccatcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggca
gcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggctctcacagcggctccggcggctctg
gcagcggcggccacgccgcagtgggcatcggagccgtgtccctgggctttctgggagcagcaggctccacaatgggagcagcctctatgaccct
gacagtgcaggccaggaatctgctgagcggcatcgtgcagcagcagtccaacctgctgagagccccagagccccagcagcacctgctgaagga
cacccactggggcatcaagcagctgcaggccagggtgctggcagtggagcactatctgagagatcagcagctgctgggcatctggggctgtagc
ggcaagctgatctgctgtaccaatgtgccctggaactctagctggtctaatcgcaacctgagcgagatctgggacaatatgacctggctgcagtg
ggataaggagatctccaactacacacagatcatctatggcctgctggaagaatctcagaatcagcaggaaaagaatgaacaggatctgctggc
actggattgataa (SEQ ID NO: 112)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCUCUCACAGCGGCUCCGGCGGCUCUGGCAGCGGCGGCC
ACGCCGCAGUGGGCAUCGGAGCCGUGUCCCUGGGCUUUCUGGGAGCAGCAGGCUCCACAAUGGGAGCA
GCCUCUAUGACCCUGACAGUGCAGGCCAGGAAUCUGCUGAGCGGCAUCGUGCAGCAGCAGUCCAACCUG
CUGAGAGCCCCAGAGCCCCAGCAGCACCUGCUGAAGGACACCCACUGGGGCAUCAAGCAGCUGCAGGCCA
GGGUGCUGGCAGUGGAGCACUAUCUGAGAGAUCAGCAGCUGCUGGGCAUCUGGGGCUGUAGCGGCAAG
CUGAUCUGCUGUACCAAUGUGCCCUGGAACUCUAGCUGGUCUAAUCGCAACCUGAGCGAGAUCUGGGA
CAAUAUGACCUGGCUGCAGUGGGAUAAGGAGAUCUCCAACUACACACAGAUCAUCUAUGGCCUGCUGGA
AGAAUCUCAGAAUCAGCAGGAAAAGAAUGAACAGGAUCUGCUGGCACUGGAUUGAUAA (SEQ ID
NO: 113)
BG505 MD39_CPG9.2 (amino acid, dna, rna)
MDWTWILFLVAAATRVHSGGNSSGSLGFLGAAGSTMGAASMTLTVQARNLLSGIVQQQSNLLRAPEPQQHLLK
DTHWGIKQLQARVLAVEHYLRDQQLLGIWGCSGKLICCTNVPWNSSWSNRNLSEIWDNMTWLNWSKEISNYTQ
IIYGLLEESQNQNESNEQDLGGNGSGGGSGSGGNGSSGLWVTVYYGVPVWKDAETTLFCASDAKAYETEKHNVW
ATHACVPTDPNPQEIHLENVTEEFNMWKNNMVEQMHEDIISLWDQSLKPCVKLTPLCVTLQCTNVTNNITDDM
RGELKNCSFNMTTELRDKKQKVYSLFYRLDVVQINENQGNRSNNSNKEYRLINCNTSAITQACPKVSFEPIPIHYCAP
AGFAILKCKDKKFNGTGPCPSVSTVQCTHGIKPVVSTQLLLNGSLAEEEVIIRSENITNNAKNILVQLNTPVQINCTRP
NNNTVKSIRIGPGQAFYYTGDIIGDIRQAHCNVSKATWNETLGKVVKQLRKHFGNNTIIRFAQSSGGDLEVTTHSFN
CGGEFFYCNTSGLFNSTWISNTSVQGSNSTGSNDSITLPCRIKQIINMWQRIGQAMYAPPIQGVIRCVSNITGLILTR
DGGSTNSTTETFRPGGGDMRDNWRSELYKYKVVKIEPLGVAPTRCNRS** (SEQ ID NO: 117)
atggattggacttggattctgttcctggtcgcagcagccacacgagtgcatagcgggggaaatagtagcggcagcctggggttcctgggagcag
caggctccaccatgggagcagcatctatgaccctgacagtgcaggccaggaatctgctgtctggcatcgtgcagcagcagagcaacctgctgag
agccccagagccccagcagcacctgctgaaggacacccactggggcatcaagcagctgcaggcccgggtgctggcagtggagcactacctgcg
cgatcagcagctgctgggaatctggggatgcagcggcaagctgatctgctgtacaaatgtgccttggaacagctcctggtccaataggaacctgt
ctgagatctgggacaatatgacctggctgaactggtctaaggagatcagcaattacacacagatcatctatggcctgctggaggagagccagaa
tcagaacgagtccaatgagcaggatctgggcggcaacggcagcggcggcggcagcggctccggcggcaacggctctagcggcctgtgggtga
ccgtgtactatggcgtgcccgtgtggaaggacgccgagactacgctgttctgcgcctccgatgccaaggcctatgagacagagaagcacaacgt
gtgggcaacccacgcatgcgtgccaacagaccctaacccacaggagatccacctggagaatgtgaccgaggagtttaacatgtggaagaaca
atatggtggagcagatgcacgaggacatcatcagcctgtgggatcagtccctgaagccttgcgtgaagctgaccccactgtgcgtgacactgca
gtgtaccaacgtgacaaacaatatcaccgacgatatgaggggcgagctgaagaattgttctttcaacatgaccacagagctgagggacaagaa
gcagaaagtgtacagcctgttttatagactggatgtggtgcagatcaatgagaaccagggcaataggagcaacaattccaacaaggagtacag
actgatcaattgcaacaccagcgccatcacacaggcctgtccaaaggtgtccttcgagcccatccctatccactattgcgcaccagcaggattcg
caatcctgaagtgtaaggataagaagtttaacggaaccggaccatgcccatctgtgagcaccgtgcagtgtacacacggcatcaagccagtggt
gtccacacagctgctgctgaatggctctctggccgaggaggaagtgatcatccggagcgagaacatcaccaacaatgccaagaatatcctggtg
cagctgaacacacccgtgcagatcaattgcacccggcctaacaataacacagtgaagtccatcaggatcggaccaggacaggccttttactata
ccggcgacatcatcggcgatatccgccaggcccactgtaacgtgagcaaggccacctggaacgagacactgggcaaggtggtgaagcagctg
aggaagcacttcggcaataacaccatcatcagatttgcacagtcctctggcggcgacctggaggtgaccacacactccttcaactgcggcggcg
agttcttttactgtaacacatctggcctgtttaatagcacctggatctctaacacaagcgtgcagggctccaattctaccggctccaacgattcta
tcacactgccctgccggatcaagcagatcatcaacatgtggcagaggatcggacaggcaatgtacgcccctcccatccagggcgtgatcagatgc
gtgagcaatatcaccggcctgatcctgacacgcgacggcggcagcaccaactccaccacagagacattcagacccggcggcggcgacatgag
ggataactggagatccgagctgtataagtataaagtcgtgaagattgagccactgggcgtcgcaccaacaagatgtaatagaagctgataa
(SEQ ID NO: 115)
AUGGAUUGGACUUGGAUUCUGUUCCUGGUCGCAGCAGCCACACGAGUGCAUAGCGGGGGAAAUAGUAG
CGGCAGCCUGGGGUUCCUGGGAGCAGCAGGCUCCACCAUGGGAGCAGCAUCUAUGACCCUGACAGUGCA
GGCCAGGAAUCUGCUGUCUGGCAUCGUGCAGCAGCAGAGCAACCUGCUGAGAGCCCCAGAGCCCCAGCA
GCACCUGCUGAAGGACACCCACUGGGGCAUCAAGCAGCUGCAGGCCCGGGUGCUGGCAGUGGAGCACUA
CCUGCGCGAUCAGCAGCUGCUGGGAAUCUGGGGAUGCAGCGGCAAGCUGAUCUGCUGUACAAAUGUGC
CUUGGAACAGCUCCUGGUCCAAUAGGAACCUGUCUGAGAUCUGGGACAAUAUGACCUGGCUGAACUGG
UCUAAGGAGAUCAGCAAUUACACACAGAUCAUCUAUGGCCUGCUGGAGGAGAGCCAGAAUCAGAACGAG
UCCAAUGAGCAGGAUCUGGGCGGCAACGGCAGCGGCGGCGGCAGCGGCUCCGGCGGCAACGGCUCUAGC
GGCCUGUGGGUGACCGUGUACUAUGGCGUGCCCGUGUGGAAGGACGCCGAGACUACGCUGUUCUGCGC
CUCCGAUGCCAAGGCCUAUGAGACAGAGAAGCACAACGUGUGGGCAACCCACGCAUGCGUGCCAACAGAC
CCUAACCCACAGGAGAUCCACCUGGAGAAUGUGACCGAGGAGUUUAACAUGUGGAAGAACAAUAUGGU
GGAGCAGAUGCACGAGGACAUCAUCAGCCUGUGGGAUCAGUCCCUGAAGCCUUGCGUGAAGCUGACCCC
ACUGUGCGUGACACUGCAGUGUACCAACGUGACAAACAAUAUCACCGACGAUAUGAGGGGCGAGCUGAA
GAAUUGUUCUUUCAACAUGACCACAGAGCUGAGGGACAAGAAGCAGAAAGUGUACAGCCUGUUUUAUA
GACUGGAUGUGGUGCAGAUCAAUGAGAACCAGGGCAAUAGGAGCAACAAUUCCAACAAGGAGUACAGAC
UGAUCAAUUGCAACACCAGCGCCAUCACACAGGCCUGUCCAAAGGUGUCCUUCGAGCCCAUCCCUAUCCA
CUAUUGCGCACCAGCAGGAUUCGCAAUCCUGAAGUGUAAGGAUAAGAAGUUUAACGGAACCGGACCAU
GCCCAUCUGUGAGCACCGUGCAGUGUACACACGGCAUCAAGCCAGUGGUGUCCACACAGCUGCUGCUGA
AUGGCUCUCUGGCCGAGGAGGAAGUGAUCAUCCGGAGCGAGAACAUCACCAACAAUGCCAAGAAUAUCC
UGGUGCAGCUGAACACACCCGUGCAGAUCAAUUGCACCCGGCCUAACAAUAACACAGUGAAGUCCAUCA
GGAUCGGACCAGGACAGGCCUUUUACUAUACCGGCGACAUCAUCGGCGAUAUCCGCCAGGCCCACUGUA
ACGUGAGCAAGGCCACCUGGAACGAGACACUGGGCAAGGUGGUGAAGCAGCUGAGGAAGCACUUCGGCA
AUAACACCAUCAUCAGAUUUGCACAGUCCUCUGGCGGCGACCUGGAGGUGACCACACACUCCUUCAACU
GCGGCGGCGAGUUCUUUUACUGUAACACAUCUGGCCUGUUUAAUAGCACCUGGAUCUCUAACACAAGC
GUGCAGGGCUCCAAUUCUACCGGCUCCAACGAUUCUAUCACACUGCCCUGCCGGAUCAAGCAGAUCAUC
AACAUGUGGCAGAGGAUCGGACAGGCAAUGUACGCCCCUCCCAUCCAGGGCGUGAUCAGAUGCGUGAGC
AAUAUCACCGGCCUGAUCCUGACACGCGACGGCGGCAGCACCAACUCCACCACAGAGACAUUCAGACCCG
GCGGCGGCGACAUGAGGGAUAACUGGAGAUCCGAGCUGUAUAAGUAUAAAGUCGUGAAGAUUGAGCCA
CUGGGCGUCGCACCAACAAGAUGUAAUAGAAGCUGAUAA (SEQ ID NO: 116)
BG505 MD39_TS 1 (amino acid, dna, rna)
There were different codon optimizations for each of the repeats
Repeat 1: human
Repeat 2: human/mouse
Repeat 3: mouse
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGSHSGSGGSGSGGHAAVGIGAVSLGFLGAAGSTMGAAS
MTLTVQARNLLSGIVQQQSNLLRAPEPQQHLLKDTHWGIKQLQARVLAVEHYLRDQQLLGIWGCSGKLICCTNVP
WNSSWSNRNLSEIWDNMTWLQWDKEISNYTQIIYGLLEESQNQQEKNEQDLLALDGGAENLWVTVYYGVPVW
KDAETTLFCASDAKAYETEKHNVWATHACVPTDPNPQEIHLENVTEEFNMWKNNMVEQMHEDIISLWDQSLKP
CVKLTPLCVTLQCTNVTNNITDDMRGELKNCSFNMTTELRDKKQKVYSLFYRLDVVQINENQGNRSNNSNKEYRLI
NCNTSAITQACPKVSFEPIPIHYCAPAGFAILKCKDKKFNGTGPCPSVSTVQCTHGIKPVVSTQLLLNGSLAEEEVIIRS
ENITNNAKNILVQLNTPVQINCTRPNNNTVKSIRIGPGQAFYYTGDIIGDIRQAHCNVSKATWNETLGKVVKQLRK
HFGNNTIIRFAQSSGGDLEVTTHSFNCGGEFFYCNTSGLFNSTWISNTSVQGSNSTGSNDSITLPCRIKQIINMWQR
IGQAMYAPPIQGVIRCVSNITGLILTRDGGSTNSTTETFRPGGGDMRDNWRSELYKYKVVKIEPLGVAPTRCKRRVV
GSHSGSGGSGSGGHAAVGIGAVSLGFLGAAGSTMGAASMTLTVQARNLLSGIVQQQSNLLRAPEPQQHLLKDTH
WGIKQLQARVLAVEHYLRDQQLLGIWGCSGKLICCTNVPWNSSWSNRNLSEIWDNMTWLQWDKEISNYTQIIY
GLLEESQNQQEKNEQDLLALDGGAENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQCTNVTNNITDDMRGELKNCSFNM
TTELRDKKQKVYSLFYRLDVVQINENQGNRSNNSNKEYRLINCNTSAITQACPKVSFEPIPIHYCAPAGFAILKCKDKK
FNGTGPCPSVSTVQCTHGIKPVVSTQLLLNGSLAEEEVIIRSENITNNAKNILVQLNTPVQINCTRPNNNTVKSIRIGP
GQAFYYTGDIIGDIRQAHCNVSKATWNETLGKVVKQLRKHFGNNTIIRFAQSSGGDLEVTTHSFNCGGEFFYCNTS
GLFNSTWISNTSVQGSNSTGSNDSITLPCRIKQIINMWQRIGQAMYAPPIQGVIRCVSNITGLILTRDGGSTNSTTET
FRPGGGDMRDNWRSELYKYKVVKIEPLGVAPTRCKRRVVGSHSGSGGSGSGGHAAVGIGAVSLGFLGAAGSTMG
AASMTLTVQARNLLSGIVQQQSNLLRAPEPQQHLLKDTHWGIKQLQARVLAVEHYLRDQQLLGIWGCSGKLICCT
NVPWNSSWSNRNLSEIWDNMTWLQWDKEISNYTQIIYGLLEESQNQQEKNEQDLLALD** (SEQ ID NO: 120)
atggactggacatggattctgttcctggtcgctgccgctacaagagtgcattccgccgaaaacctgtgggtcaccgtctactatggagtgcccgtg
tggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccg
ccatcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggca
gcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggctctcacagcggctccggcggctctg
gcagcggcggccacgccgcagtgggcatcggagccgtgtccctgggctttctgggagcagcaggctccacaatgggagcagcctctatgaccct
gacagtgcaggccaggaatctgctgagcggcatcgtgcagcagcagtccaacctgctgagagccccagagccccagcagcacctgctgaagga
cacccactggggcatcaagcagctgcaggccagggtgctggcagtggagcactatctgagagatcagcagctgctgggcatctggggctgtagc
ggcaagctgatctgctgtaccaatgtgccctggaactctagctggtctaatcgcaacctgagcgagatctgggacaatatgacctggctgcagtg
ggataaggagatctccaactacacacagatcatctatggcctgctggaagaatctcagaatcagcaggaaaagaatgaacaggatctgctggc
actggatggcggcgccgaaaacctgtgggtcaccgtgtactacggagtccccgtgtggaaagatgcagagacaaccctgttctgcgcttccgac
gctaaagcttacgagacagaaaaacacaacgtgtgggccactcatgcctgcgtgcctacagaccctaacccacaggaaatccacctggagaat
gtgacggaggagtttaacatgtggaagaataacatggtcgagcagatgcatgaagatatcatttccttatgggaccaatccctgaagccttgcgt
gaagctgaccccactgtgcgtgacactgcaatgcactaacgtgaccaataacattaccgacgatatgcgcggcgagctgaagaactgctctttc
aacatgactaccgagctgagagataagaaacagaaagtgtacagcctgttttatcggttagatgtggtgcagatcaatgaaaaccagggcaat
cggtccaacaattctaacaaggaatatcgcctgatcaattgtaacacctccgccattacccaggcttgccctaaggtgtctttcgagcccatccct
atccactattgcgccccagctggatttgctatcctgaagtgtaaggacaaaaagtttaacgggaccggaccatgtcctagcgtgtccactgtgca
gtgcacccatggcatcaagcctgtggtgtccacccaacttctgctgaatggctctctggctgaagaagaagtgatcattaggtccgaaaatattac
taataacgctaaaaatatcctggtccagctgaacacgcctgtccagatcaattgtacccggccaaataacaacacagtgaagtctatcagaatc
ggcccaggccaggccttctactacacaggcgacattatcggcgatattcgccaggcccactgtaatgtgagcaaagctacatggaatgagacac
tgggcaaggtagtcaaacagctgagaaaacattttggaaacaacaccatcatccgctttgcacagtctagcggcggcgacctggaggtaactac
ccacagcttcaattgtggcggcgagttcttttactgtaataccagcggcctgtttaatagtacttggatcagcaacacatctgtgcagggctctaa
ctccactggctctaacgatagcatcacactgccttgtcggatcaagcaaatcatcaacatgtggcaaaggattgggcaggctatgtatgcccctcc
aatccagggcgtgatccggtgcgtgagcaacattacaggcctgatcctgacaagagacggcggctccaccaactctactaccgagacattccgg
cccggcggcggcgacatgcgtgataactggcgcagcgaactgtataaatataaagtggtgaagatcgagcctctgggcgtggccccaactaggt
gtaaaagaagggtcgtcggctcccacagcggcagcggcggctccggctctggcggccacgcggctgtcggcatcggcgccgtgagcctgggctt
tctgggcgccgccggctccactatgggcgcagcctctatgaccctgactgtccaggctagaaatctgctgtctggaatcgtgcagcagcagtcta
acctgctgagggcacctgagccacaacagcacctgctgaaggatacacattggggcatcaagcagttacaagccagggtgctggccgtggaac
actacctgcgcgatcagcaattactgggcatttggggatgctctggcaagctgatttgttgcaccaatgtgccctggaactcctcttggagcaaca
gaaacctgtccgaaatctgggataacatgacatggctgcagtgggacaaggaaatttccaattatacccagatcatctatggactgctggaaga
aagtcagaatcagcaggagaagaatgaacaggatctgctggcactggatggcggcgccgaaaacctgtgggtcaccgtgtattatggagtgcc
agtgtggaaggacgccgagaccacactgttttgtgcctctgatgccaaggcctacgagaccgagaagcacaacgtgtgggccacccacgcctgc
gtgcccacagacccaaatcctcaggagatccacctggagaacgtgaccgaggagtttaacatgtggaagaacaatatggtggagcagatgcac
gaggatatcatctctctgtgggatcagtctctgaagccatgtgtgaagctgaccccactgtgcgtgaccctgcagtgtacaaatgtgacaaacaa
catcacagatgacatgagaggcgagctgaagaactgttccttcaatatgaccaccgagctgagagacaagaagcagaaggtgtattctctgttt
taccggctggacgtggtgcagatcaacgagaatcagggcaatcggtctaacaactccaataaggagtatagactgatcaactgcaacacctctg
ccatcacccaggcctgtcctaaggtgtcctttgagccaatcccaatccactattgcgcccctgccggctttgccatcctgaagtgcaaggacaaga
agtttaacggcacaggcccctgcccatccgtgagcacagtgcagtgtacccacggcatcaagcctgtggtgtccacccagctgctgctgaacggc
tccctggccgaggaggaggtaatcatcaggtctgagaacatcacaaataacgccaagaacatcctggtgcagctgaacaccccagtgcagatc
aactgtacccggcctaacaataataccgtgaagtctatccggatcggcccaggccaggccttctactataccggcgatatcatcggcgatatcag
acaggcccactgcaacgtgtccaaggccacatggaacgagacactgggcaaggtggtgaagcagctgcggaagcactttggcaataacacca
tcatcagattcgcccagtcttccggcggcgacctggaggtgacaacccactccttcaattgcggcggcgagttcttttactgtaatacaagcggcc
tgtttaatagcacctggatctctaacacctccgtgcagggctccaacagcacaggctctaatgattccatcaccctgccttgccggatcaagcaga
tcatcaatatgtggcagagaatcggccaggccatgtatgcccctccaatccagggcgtgatccgctgcgtgtccaacatcacaggcctgatcctg
acaagagatggcggctccaccaacagcaccacagagaccttcagacccggcggcggcgacatgcgcgacaactggagatccgagctgtataa
gtacaaggtggtgaagatcgagcccctgggcgtggccccaacccggtgtaagcgcagagtggtgggcagccacagcggcagcggcggcagcg
gctccggcggccacgccgccgtgggcatcggcgccgtgtccctgggcttcctgggcgccgccggctccaccatgggcgccgcctccatgacactg
acagtgcaggccagaaatctgctgtccggcatcgtgcagcagcagtccaatctgctgcgggcccctgagccacagcagcacctgctgaaggata
cccactggggcatcaagcagctgcaggcccgggtgctggccgtggagcactacctgagggatcagcagctgctgggcatctggggctgttccgg
caagctgatctgctgtacaaacgtgccctggaacagctcctggtccaataggaacctgtccgagatctgggataacatgacctggctgcagtggg
ataaggagatcagcaactacacacagatcatctacggcctgctggaggagagccagaatcagcaggagaagaacgagcaggacctgctggcc
ctggattgataa (SEQ ID NO: 118)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCUCUCACAGCGGCUCCGGCGGCUCUGGCAGCGGCGGCC
ACGCCGCAGUGGGCAUCGGAGCCGUGUCCCUGGGCUUUCUGGGAGCAGCAGGCUCCACAAUGGGAGCA
GCCUCUAUGACCCUGACAGUGCAGGCCAGGAAUCUGCUGAGCGGCAUCGUGCAGCAGCAGUCCAACCUG
CUGAGAGCCCCAGAGCCCCAGCAGCACCUGCUGAAGGACACCCACUGGGGCAUCAAGCAGCUGCAGGCCA
GGGUGCUGGCAGUGGAGCACUAUCUGAGAGAUCAGCAGCUGCUGGGCAUCUGGGGCUGUAGCGGCAAG
CUGAUCUGCUGUACCAAUGUGCCCUGGAACUCUAGCUGGUCUAAUCGCAACCUGAGCGAGAUCUGGGA
CAAUAUGACCUGGCUGCAGUGGGAUAAGGAGAUCUCCAACUACACACAGAUCAUCUAUGGCCUGCUGGA
AGAAUCUCAGAAUCAGCAGGAAAAGAAUGAACAGGAUCUGCUGGCACUGGAUGGCGGCGCCGAAAACCU
GUGGGUCACCGUGUACUACGGAGUCCCCGUGUGGAAAGAUGCAGAGACAACCCUGUUCUGCGCUUCCG
ACGCUAAAGCUUACGAGACAGAAAAACACAACGUGUGGGCCACUCAUGCCUGCGUGCCUACAGACCCUAA
CCCACAGGAAAUCCACCUGGAGAAUGUGACGGAGGAGUUUAACAUGUGGAAGAAUAACAUGGUCGAGC
AGAUGCAUGAAGAUAUCAUUUCCUUAUGGGACCAAUCCCUGAAGCCUUGCGUGAAGCUGACCCCACUGU
GCGUGACACUGCAAUGCACUAACGUGACCAAUAACAUUACCGACGAUAUGCGCGGCGAGCUGAAGAACU
GCUCUUUCAACAUGACUACCGAGCUGAGAGAUAAGAAACAGAAAGUGUACAGCCUGUUUUAUCGGUUA
GAUGUGGUGCAGAUCAAUGAAAACCAGGGCAAUCGGUCCAACAAUUCUAACAAGGAAUAUCGCCUGAUC
AAUUGUAACACCUCCGCCAUUACCCAGGCUUGCCCUAAGGUGUCUUUCGAGCCCAUCCCUAUCCACUAU
UGCGCCCCAGCUGGAUUUGCUAUCCUGAAGUGUAAGGACAAAAAGUUUAACGGGACCGGACCAUGUCC
UAGCGUGUCCACUGUGCAGUGCACCCAUGGCAUCAAGCCUGUGGUGUCCACCCAACUUCUGCUGAAUGG
CUCUCUGGCUGAAGAAGAAGUGAUCAUUAGGUCCGAAAAUAUUACUAAUAACGCUAAAAAUAUCCUGG
UCCAGCUGAACACGCCUGUCCAGAUCAAUUGUACCCGGCCAAAUAACAACACAGUGAAGUCUAUCAGAA
UCGGCCCAGGCCAGGCCUUCUACUACACAGGCGACAUUAUCGGCGAUAUUCGCCAGGCCCACUGUAAUG
UGAGCAAAGCUACAUGGAAUGAGACACUGGGCAAGGUAGUCAAACAGCUGAGAAAACAUUUUGGAAAC
AACACCAUCAUCCGCUUUGCACAGUCUAGCGGCGGCGACCUGGAGGUAACUACCCACAGCUUCAAUUGU
GGCGGCGAGUUCUUUUACUGUAAUACCAGCGGCCUGUUUAAUAGUACUUGGAUCAGCAACACAUCUGU
GCAGGGCUCUAACUCCACUGGCUCUAACGAUAGCAUCACACUGCCUUGUCGGAUCAAGCAAAUCAUCAA
CAUGUGGCAAAGGAUUGGGCAGGCUAUGUAUGCCCCUCCAAUCCAGGGCGUGAUCCGGUGCGUGAGCA
ACAUUACAGGCCUGAUCCUGACAAGAGACGGCGGCUCCACCAACUCUACUACCGAGACAUUCCGGCCCGG
CGGCGGCGACAUGCGUGAUAACUGGCGCAGCGAACUGUAUAAAUAUAAAGUGGUGAAGAUCGAGCCUC
UGGGCGUGGCCCCAACUAGGUGUAAAAGAAGGGUCGUCGGCUCCCACAGCGGCAGCGGCGGCUCCGGCU
CUGGCGGCCACGCGGCUGUCGGCAUCGGCGCCGUGAGCCUGGGCUUUCUGGGCGCCGCCGGCUCCACUA
UGGGCGCAGCCUCUAUGACCCUGACUGUCCAGGCUAGAAAUCUGCUGUCUGGAAUCGUGCAGCAGCAG
UCUAACCUGCUGAGGGCACCUGAGCCACAACAGCACCUGCUGAAGGAUACACAUUGGGGCAUCAAGCAG
UUACAAGCCAGGGUGCUGGCCGUGGAACACUACCUGCGCGAUCAGCAAUUACUGGGCAUUUGGGGAUG
CUCUGGCAAGCUGAUUUGUUGCACCAAUGUGCCCUGGAACUCCUCUUGGAGCAACAGAAACCUGUCCGA
AAUCUGGGAUAACAUGACAUGGCUGCAGUGGGACAAGGAAAUUUCCAAUUAUACCCAGAUCAUCUAUG
GACUGCUGGAAGAAAGUCAGAAUCAGCAGGAGAAGAAUGAACAGGAUCUGCUGGCACUGGAUGGCGGC
GCCGAAAACCUGUGGGUCACCGUGUAUUAUGGAGUGCCAGUGUGGAAGGACGCCGAGACCACACUGUU
UUGUGCCUCUGAUGCCAAGGCCUACGAGACCGAGAAGCACAACGUGUGGGCCACCCACGCCUGCGUGCC
CACAGACCCAAAUCCUCAGGAGAUCCACCUGGAGAACGUGACCGAGGAGUUUAACAUGUGGAAGAACAA
UAUGGUGGAGCAGAUGCACGAGGAUAUCAUCUCUCUGUGGGAUCAGUCUCUGAAGCCAUGUGUGAAGC
UGACCCCACUGUGCGUGACCCUGCAGUGUACAAAUGUGACAAACAACAUCACAGAUGACAUGAGAGGCG
AGCUGAAGAACUGUUCCUUCAAUAUGACCACCGAGCUGAGAGACAAGAAGCAGAAGGUGUAUUCUCUG
UUUUACCGGCUGGACGUGGUGCAGAUCAACGAGAAUCAGGGCAAUCGGUCUAACAACUCCAAUAAGGA
GUAUAGACUGAUCAACUGCAACACCUCUGCCAUCACCCAGGCCUGUCCUAAGGUGUCCUUUGAGCCAAU
CCCAAUCCACUAUUGCGCCCCUGCCGGCUUUGCCAUCCUGAAGUGCAAGGACAAGAAGUUUAACGGCAC
AGGCCCCUGCCCAUCCGUGAGCACAGUGCAGUGUACCCACGGCAUCAAGCCUGUGGUGUCCACCCAGCU
GCUGCUGAACGGCUCCCUGGCCGAGGAGGAGGUAAUCAUCAGGUCUGAGAACAUCACAAAUAACGCCAA
GAACAUCCUGGUGCAGCUGAACACCCCAGUGCAGAUCAACUGUACCCGGCCUAACAAUAAUACCGUGAA
GUCUAUCCGGAUCGGCCCAGGCCAGGCCUUCUACUAUACCGGCGAUAUCAUCGGCGAUAUCAGACAGGC
CCACUGCAACGUGUCCAAGGCCACAUGGAACGAGACACUGGGCAAGGUGGUGAAGCAGCUGCGGAAGCA
CUUUGGCAAUAACACCAUCAUCAGAUUCGCCCAGUCUUCCGGCGGCGACCUGGAGGUGACAACCCACUCC
UUCAAUUGCGGCGGCGAGUUCUUUUACUGUAAUACAAGCGGCCUGUUUAAUAGCACCUGGAUCUCUAA
CACCUCCGUGCAGGGCUCCAACAGCACAGGCUCUAAUGAUUCCAUCACCCUGCCUUGCCGGAUCAAGCAG
AUCAUCAAUAUGUGGCAGAGAAUCGGCCAGGCCAUGUAUGCCCCUCCAAUCCAGGGCGUGAUCCGCUGC
GUGUCCAACAUCACAGGCCUGAUCCUGACAAGAGAUGGCGGCUCCACCAACAGCACCACAGAGACCUUCA
GACCCGGCGGCGGCGACAUGCGCGACAACUGGAGAUCCGAGCUGUAUAAGUACAAGGUGGUGAAGAUC
GAGCCCCUGGGCGUGGCCCCAACCCGGUGUAAGCGCAGAGUGGUGGGCAGCCACAGCGGCAGCGGCGGC
AGCGGCUCCGGCGGCCACGCCGCCGUGGGCAUCGGCGCCGUGUCCCUGGGCUUCCUGGGCGCCGCCGGC
UCCACCAUGGGCGCCGCCUCCAUGACACUGACAGUGCAGGCCAGAAAUCUGCUGUCCGGCAUCGUGCAG
CAGCAGUCCAAUCUGCUGCGGGCCCCUGAGCCACAGCAGCACCUGCUGAAGGAUACCCACUGGGGCAUCA
AGCAGCUGCAGGCCCGGGUGCUGGCCGUGGAGCACUACCUGAGGGAUCAGCAGCUGCUGGGCAUCUGG
GGCUGUUCCGGCAAGCUGAUCUGCUGUACAAACGUGCCCUGGAACAGCUCCUGGUCCAAUAGGAACCUG
UCCGAGAUCUGGGAUAACAUGACCUGGCUGCAGUGGGAUAAGGAGAUCAGCAACUACACACAGAUCAUC
UACGGCCUGCUGGAGGAGAGCCAGAAUCAGCAGGAGAAGAACGAGCAGGACCUGCUGGCCCUGGAUUG
AUAA (SEQ ID NO: 119)
BG505 MD39_TS 2 (amino acid, dna, rna)
(longer linker)
There were different codon optimizations for each of the repeats
Repeat 1: human
Repeat 2: human/mouse
Repeat 3: mouse
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGSHSGSGGSGSGGHAAVGIGAVSLGFLGAAGSTMGAAS
MTLTVQARNLLSGIVQQQSNLLRAPEPQQHLLKDTHWGIKQLQARVLAVEHYLRDQQLLGIWGCSGKLICCTNVP
WNSSWSNRNLSEIWDNMTWLQWDKEISNYTQIIYGLLEESQNQQEKNEQDLLALDGGSGSGAENLWVTVYYGV
PVWKDAETTLFCASDAKAYETEKHNVWATHACVPTDPNPQEIHLENVTEEFNMWKNNMVEQMHEDIISLWDQ
SLKPCVKLTPLCVTLQCTNVTNNITDDMRGELKNCSFNMTTELRDKKQKVYSLFYRLDVVQINENQGNRSNNSNKE
YRLINCNTSAITQACPKVSFEPIPIHYCAPAGFAILKCKDKKFNGTGPCPSVSTVQCTHGIKPVVSTQLLLNGSLAEEEV
IIRSENITNNAKNILVQLNTPVQINCTRPNNNTVKSIRIGPGQAFYYTGDIIGDIRQAHCNVSKATWNETLGKVVKQL
RKHFGNNTIIRFAQSSGGDLEVTTHSFNCGGEFFYCNTSGLFNSTWISNTSVQGSNSTGSNDSITLPCRIKQIINMW
QRIGQAMYAPPIQGVIRCVSNITGLILTRDGGSTNSTTETFRPGGGDMRDNWRSELYKYKVVKIEPLGVAPTRCKR
RVVGSHSGSGGSGSGGHAAVGIGAVSLGFLGAAGSTMGAASMTLTVQARNLLSGIVQQQSNLLRAPEPQQHLLK
DTHWGIKQLQARVLAVEHYLRDQQLLGIWGCSGKLICCTNVPWNSSWSNRNLSEIWDNMTWLQWDKEISNYT
QIIYGLLEESQNQQEKNEQDLLALDGGSGSGAENLWVTVYYGVPVWKDAETTLFCASDAKAYETEKHNVWATHA
CVPTDPNPQEIHLENVTEEFNMWKNNMVEQMHEDIISLWDQSLKPCVKLTPLCVTLQCTNVTNNITDDMRGELK
NCSFNMTTELRDKKQKVYSLFYRLDVVQINENQGNRSNNSNKEYRLINCNTSAITQACPKVSFEPIPIHYCAPAGFAI
LKCKDKKFNGTGPCPSVSTVQCTHGIKPVVSTQLLLNGSLAEEEVIIRSENITNNAKNILVQLNTPVQINCTRPNNNT
VKSIRIGPGQAFYYTGDIIGDIRQAHCNVSKATWNETLGKVVKQLRKHFGNNTIIRFAQSSGGDLEVTTHSFNCGGE
FFYCNTSGLFNSTWISNTSVQGSNSTGSNDSITLPCRIKQIINMWQRIGQAMYAPPIQGVIRCVSNITGLILTRDGGS
TNSTTETFRPGGGDMRDNWRSELYKYKVVKIEPLGVAPTRCKRRVVGSHSGSGGSGSGGHAAVGIGAVSLGFLGA
AGSTMGAASMTLTVQARNLLSGIVQQQSNLLRAPEPQQHLLKDTHWGIKQLQARVLAVEHYLRDQQLLGIWGCS
GKLICCTNVPWNSSWSNRNLSEIWDNMTWLQWDKEISNYTQIIYGLLEESQNQQEKNEQDLLALD** (SEQ ID
NO: 123)
atggactggacatggattctgttcctggtcgctgccgctacaagagtgcattccgccgaaaacctgtgggtcaccgtctactatggagtgcccgtg
tggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccg
ccatcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggca
gcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggctctcacagcggctccggcggctctg
gcagcggcggccacgccgcagtgggcatcggagccgtgtccctgggctttctgggagcagcaggctccacaatgggagcagcctctatgaccct
gacagtgcaggccaggaatctgctgagcggcatcgtgcagcagcagtccaacctgctgagagccccagagccccagcagcacctgctgaagga
cacccactggggcatcaagcagctgcaggccagggtgctggcagtggagcactatctgagagatcagcagctgctgggcatctggggctgtagc
ggcaagctgatctgctgtaccaatgtgccctggaactctagctggtctaatcgcaacctgagcgagatctgggacaatatgacctggctgcagtg
ggataaggagatctccaactacacacagatcatctatggcctgctggaagaatctcagaatcagcaggaaaagaatgaacaggatctgctggc
actggatggcggcagcggcagcggcgccgaaaacctgtgggtcaccgtgtactacggagtccccgtgtggaaagatgcagagacaaccctgtt
ctgcgcttccgacgctaaagcttacgagacagaaaaacacaacgtgtgggccactcatgcctgcgtgcctacagaccctaacccacaggaaatc
cacctggagaatgtgacggaggagtttaacatgtggaagaataacatggtcgagcagatgcatgaagatatcatttccttatgggaccaatccct
gaagccttgcgtgaagctgaccccactgtgcgtgacactgcaatgcactaacgtgaccaataacattaccgacgatatgcgcggcgagctgaag
aactgctctttcaacatgactaccgagctgagagataagaaacagaaagtgtacagcctgttttatcggttagatgtggtgcagatcaatgaaaa
ccagggcaatcggtccaacaattctaacaaggaatatcgcctgatcaattgtaacacctccgccattacccaggcttgccctaaggtgtctttcga
gcccatccctatccactattgcgccccagctggatttgctatcctgaagtgtaaggacaaaaagtttaacgggaccggaccatgtcctagcgtgtc
cactgtgcagtgcacccatggcatcaagcctgtggtgtccacccaacttctgctgaatggctctctggctgaagaagaagtgatcattaggtccga
aaatattactaataacgctaaaaatatcctggtccagctgaacacgcctgtccagatcaattgtacccggccaaataacaacacagtgaagtcta
tcagaatcggcccaggccaggccttctactacacaggcgacattatcggcgatattcgccaggcccactgtaatgtgagcaaagctacatggaa
tgagacactgggcaaggtagtcaaacagctgagaaaacattttggaaacaacaccatcatccgctttgcacagtctagcggcggcgacctggagg
taactacccacagcttcaattgtggcggcgagttcttttactgtaataccagcggcctgtttaatagtacttggatcagcaacacatctgtgcag
ggctctaactccactggctctaacgatagcatcacactgccttgtcggatcaagcaaatcatcaacatgtggcaaaggattgggcaggctatgta
tgcccctccaatccagggcgtgatccggtgcgtgagcaacattacaggcctgatcctgacaagagacggcggctccaccaactctactaccgag
acattccggcccggcggcggcgacatgcgtgataactggcgcagcgaactgtataaatataaagtggtgaagatcgagcctctgggcgtggccc
caactaggtgtaaaagaagggtcgtcggctcccacagcggcagcggcggctccggctctggcggccacgcggctgtcggcatcggcgccgtgagc
ctgggctttctgggcgccgccggctccactatgggcgcagcctctatgaccctgactgtccaggctagaaatctgctgtctggaatcgtgcagc
agcagtctaacctgctgagggcacctgagccacaacagcacctgctgaaggatacacattggggcatcaagcagttacaagccagggtgctggcc
gtggaacactacctgcgcgatcagcaattactgggcatttggggatgctctggcaagctgatttgttgcaccaatgtgccctggaactcctctt
ggagcaacagaaacctgtccgaaatctgggataacatgacatggctgcagtgggacaaggaaatttccaattatacccagatcatctatggact
gctggaagaaagtcagaatcagcaggagaagaatgaacaggatctgctggcactggatggcggcagcggcagcggcgccgaaaacctgtgg
gtcaccgtgtattatggagtgccagtgtggaaggacgccgagaccacactgttttgtgcctctgatgccaaggcctacgagaccgagaagcaca
acgtgtgggccacccacgcctgcgtgcccacagacccaaatcctcaggagatccacctggagaacgtgaccgaggagtttaacatgtggaaga
acaatatggtggagcagatgcacgaggatatcatctctctgtgggatcagtctctgaagccatgtgtgaagctgaccccactgtgcgtgaccctg
cagtgtacaaatgtgacaaacaacatcacagatgacatgagaggcgagctgaagaactgttccttcaatatgaccaccgagctgagagacaag
aagcagaaggtgtattctctgttttaccggctggacgtggtgcagatcaacgagaatcagggcaatcggtctaacaactccaataaggagtataga
ctgatcaactgcaacacctctgccatcacccaggcctgtcctaaggtgtcctttgagccaatcccaatccactattgcgcccctgccggctttgc
catcctgaagtgcaaggacaagaagtttaacggcacaggcccctgcccatccgtgagcacagtgcagtgtacccacggcatcaagcctgtggtg
tccacccagctgctgctgaacggctccctggccgaggaggaggtaatcatcaggtctgagaacatcacaaataacgccaagaacatcctggtgc
agctgaacaccccagtgcagatcaactgtacccggcctaacaataataccgtgaagtctatccggatcggcccaggccaggccttctactatacc
ggcgatatcatcggcgatatcagacaggcccactgcaacgtgtccaaggccacatggaacgagacactgggcaaggtggtgaagcagctgcg
gaagcactttggcaataacaccatcatcagattcgcccagtcttccggcggcgacctggaggtgacaacccactccttcaattgcggcggcgagtt
cttttactgtaatacaagcggcctgtttaatagcacctggatctctaacacctccgtgcagggctccaacagcacaggctctaatgattccatcac
cctgccttgccggatcaagcagatcatcaatatgtggcagagaatcggccaggccatgtatgcccctccaatccagggcgtgatccgctgcgtgt
ccaacatcacaggcctgatcctgacaagagatggcggctccaccaacagcaccacagagaccttcagacccggcggcggcgacatgcgcgac
aactggagatccgagctgtataagtacaaggtggtgaagatcgagcccctgggcgtggccccaacccggtgtaagcgcagagtggtgggcagc
cacagcggcagcggcggcagcggctccggcggccacgccgccgtgggcatcggcgccgtgtccctgggcttcctgggcgccgccggctccacc
atgggcgccgcctccatgacactgacagtgcaggccagaaatctgctgtccggcatcgtgcagcagcagtccaatctgctgcgggcccctgagc
cacagcagcacctgctgaaggatacccactggggcatcaagcagctgcaggcccgggtgctggccgtggagcactacctgagggatcagcagc
tgctgggcatctggggctgttccggcaagctgatctgctgtacaaacgtgccctggaacagctcctggtccaataggaacctgtccgagatctgg
gataacatgacctggctgcagtgggataaggagatcagcaactacacacagatcatctacggcctgctggaggagagccagaatcagcagga
gaagaacgagcaggacctgctggccctggattgataa (SEQ ID NO: 121)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCUCUCACAGCGGCUCCGGCGGCUCUGGCAGCGGCGGCC
ACGCCGCAGUGGGCAUCGGAGCCGUGUCCCUGGGCUUUCUGGGAGCAGCAGGCUCCACAAUGGGAGCA
GCCUCUAUGACCCUGACAGUGCAGGCCAGGAAUCUGCUGAGCGGCAUCGUGCAGCAGCAGUCCAACCUG
CUGAGAGCCCCAGAGCCCCAGCAGCACCUGCUGAAGGACACCCACUGGGGCAUCAAGCAGCUGCAGGCCA
GGGUGCUGGCAGUGGAGCACUAUCUGAGAGAUCAGCAGCUGCUGGGCAUCUGGGGCUGUAGCGGCAAG
CUGAUCUGCUGUACCAAUGUGCCCUGGAACUCUAGCUGGUCUAAUCGCAACCUGAGCGAGAUCUGGGA
CAAUAUGACCUGGCUGCAGUGGGAUAAGGAGAUCUCCAACUACACACAGAUCAUCUAUGGCCUGCUGGA
AGAAUCUCAGAAUCAGCAGGAAAAGAAUGAACAGGAUCUGCUGGCACUGGAUGGCGGCAGCGGCAGCG
GCGCCGAAAACCUGUGGGUCACCGUGUACUACGGAGUCCCCGUGUGGAAAGAUGCAGAGACAACCCUGU
UCUGCGCUUCCGACGCUAAAGCUUACGAGACAGAAAAACACAACGUGUGGGCCACUCAUGCCUGCGUGC
CUACAGACCCUAACCCACAGGAAAUCCACCUGGAGAAUGUGACGGAGGAGUUUAACAUGUGGAAGAAUA
ACAUGGUCGAGCAGAUGCAUGAAGAUAUCAUUUCCUUAUGGGACCAAUCCCUGAAGCCUUGCGUGAAG
CUGACCCCACUGUGCGUGACACUGCAAUGCACUAACGUGACCAAUAACAUUACCGACGAUAUGCGCGGC
GAGCUGAAGAACUGCUCUUUCAACAUGACUACCGAGCUGAGAGAUAAGAAACAGAAAGUGUACAGCCUG
UUUUAUCGGUUAGAUGUGGUGCAGAUCAAUGAAAACCAGGGCAAUCGGUCCAACAAUUCUAACAAGGA
AUAUCGCCUGAUCAAUUGUAACACCUCCGCCAUUACCCAGGCUUGCCCUAAGGUGUCUUUCGAGCCCAU
CCCUAUCCACUAUUGCGCCCCAGCUGGAUUUGCUAUCCUGAAGUGUAAGGACAAAAAGUUUAACGGGAC
CGGACCAUGUCCUAGCGUGUCCACUGUGCAGUGCACCCAUGGCAUCAAGCCUGUGGUGUCCACCCAACU
UCUGCUGAAUGGCUCUCUGGCUGAAGAAGAAGUGAUCAUUAGGUCCGAAAAUAUUACUAAUAACGCUA
AAAAUAUCCUGGUCCAGCUGAACACGCCUGUCCAGAUCAAUUGUACCCGGCCAAAUAACAACACAGUGAA
GUCUAUCAGAAUCGGCCCAGGCCAGGCCUUCUACUACACAGGCGACAUUAUCGGCGAUAUUCGCCAGGC
CCACUGUAAUGUGAGCAAAGCUACAUGGAAUGAGACACUGGGCAAGGUAGUCAAACAGCUGAGAAAACA
UUUUGGAAACAACACCAUCAUCCGCUUUGCACAGUCUAGCGGCGGCGACCUGGAGGUAACUACCCACAG
CUUCAAUUGUGGCGGCGAGUUCUUUUACUGUAAUACCAGCGGCCUGUUUAAUAGUACUUGGAUCAGCA
ACACAUCUGUGCAGGGCUCUAACUCCACUGGCUCUAACGAUAGCAUCACACUGCCUUGUCGGAUCAAGC
AAAUCAUCAACAUGUGGCAAAGGAUUGGGCAGGCUAUGUAUGCCCCUCCAAUCCAGGGCGUGAUCCGG
UGCGUGAGCAACAUUACAGGCCUGAUCCUGACAAGAGACGGCGGCUCCACCAACUCUACUACCGAGACA
UUCCGGCCCGGCGGCGGCGACAUGCGUGAUAACUGGCGCAGCGAACUGUAUAAAUAUAAAGUGGUGAA
GAUCGAGCCUCUGGGCGUGGCCCCAACUAGGUGUAAAAGAAGGGUCGUCGGCUCCCACAGCGGCAGCGG
CGGCUCCGGCUCUGGCGGCCACGCGGCUGUCGGCAUCGGCGCCGUGAGCCUGGGCUUUCUGGGCGCCGC
CGGCUCCACUAUGGGCGCAGCCUCUAUGACCCUGACUGUCCAGGCUAGAAAUCUGCUGUCUGGAAUCGU
GCAGCAGCAGUCUAACCUGCUGAGGGCACCUGAGCCACAACAGCACCUGCUGAAGGAUACACAUUGGGG
CAUCAAGCAGUUACAAGCCAGGGUGCUGGCCGUGGAACACUACCUGCGCGAUCAGCAAUUACUGGGCAU
UUGGGGAUGCUCUGGCAAGCUGAUUUGUUGCACCAAUGUGCCCUGGAACUCCUCUUGGAGCAACAGAA
ACCUGUCCGAAAUCUGGGAUAACAUGACAUGGCUGCAGUGGGACAAGGAAAUUUCCAAUUAUACCCAGA
UCAUCUAUGGACUGCUGGAAGAAAGUCAGAAUCAGCAGGAGAAGAAUGAACAGGAUCUGCUGGCACUG
GAUGGCGGCAGCGGCAGCGGCGCCGAAAACCUGUGGGUCACCGUGUAUUAUGGAGUGCCAGUGUGGAA
GGACGCCGAGACCACACUGUUUUGUGCCUCUGAUGCCAAGGCCUACGAGACCGAGAAGCACAACGUGUG
GGCCACCCACGCCUGCGUGCCCACAGACCCAAAUCCUCAGGAGAUCCACCUGGAGAACGUGACCGAGGAG
UUUAACAUGUGGAAGAACAAUAUGGUGGAGCAGAUGCACGAGGAUAUCAUCUCUCUGUGGGAUCAGUC
UCUGAAGCCAUGUGUGAAGCUGACCCCACUGUGCGUGACCCUGCAGUGUACAAAUGUGACAAACAACAU
CACAGAUGACAUGAGAGGCGAGCUGAAGAACUGUUCCUUCAAUAUGACCACCGAGCUGAGAGACAAGAA
GCAGAAGGUGUAUUCUCUGUUUUACCGGCUGGACGUGGUGCAGAUCAACGAGAAUCAGGGCAAUCGGU
CUAACAACUCCAAUAAGGAGUAUAGACUGAUCAACUGCAACACCUCUGCCAUCACCCAGGCCUGUCCUAA
GGUGUCCUUUGAGCCAAUCCCAAUCCACUAUUGCGCCCCUGCCGGCUUUGCCAUCCUGAAGUGCAAGGA
CAAGAAGUUUAACGGCACAGGCCCCUGCCCAUCCGUGAGCACAGUGCAGUGUACCCACGGCAUCAAGCCU
GUGGUGUCCACCCAGCUGCUGCUGAACGGCUCCCUGGCCGAGGAGGAGGUAAUCAUCAGGUCUGAGAA
CAUCACAAAUAACGCCAAGAACAUCCUGGUGCAGCUGAACACCCCAGUGCAGAUCAACUGUACCCGGCCU
AACAAUAAUACCGUGAAGUCUAUCCGGAUCGGCCCAGGCCAGGCCUUCUACUAUACCGGCGAUAUCAUC
GGCGAUAUCAGACAGGCCCACUGCAACGUGUCCAAGGCCACAUGGAACGAGACACUGGGCAAGGUGGUG
AAGCAGCUGCGGAAGCACUUUGGCAAUAACACCAUCAUCAGAUUCGCCCAGUCUUCCGGCGGCGACCUG
GAGGUGACAACCCACUCCUUCAAUUGCGGCGGCGAGUUCUUUUACUGUAAUACAAGCGGCCUGUUUAA
UAGCACCUGGAUCUCUAACACCUCCGUGCAGGGCUCCAACAGCACAGGCUCUAAUGAUUCCAUCACCCUG
CCUUGCCGGAUCAAGCAGAUCAUCAAUAUGUGGCAGAGAAUCGGCCAGGCCAUGUAUGCCCCUCCAAUC
CAGGGCGUGAUCCGCUGCGUGUCCAACAUCACAGGCCUGAUCCUGACAAGAGAUGGCGGCUCCACCAAC
AGCACCACAGAGACCUUCAGACCCGGCGGCGGCGACAUGCGCGACAACUGGAGAUCCGAGCUGUAUAAG
UACAAGGUGGUGAAGAUCGAGCCCCUGGGCGUGGCCCCAACCCGGUGUAAGCGCAGAGUGGUGGGCAG
CCACAGCGGCAGCGGCGGCAGCGGCUCCGGCGGCCACGCCGCCGUGGGCAUCGGCGCCGUGUCCCUGGG
CUUCCUGGGCGCCGCCGGCUCCACCAUGGGCGCCGCCUCCAUGACACUGACAGUGCAGGCCAGAAAUCUG
CUGUCCGGCAUCGUGCAGCAGCAGUCCAAUCUGCUGCGGGCCCCUGAGCCACAGCAGCACCUGCUGAAG
GAUACCCACUGGGGCAUCAAGCAGCUGCAGGCCCGGGUGCUGGCCGUGGAGCACUACCUGAGGGAUCAG
CAGCUGCUGGGCAUCUGGGGCUGUUCCGGCAAGCUGAUCUGCUGUACAAACGUGCCCUGGAACAGCUCC
UGGUCCAAUAGGAACCUGUCCGAGAUCUGGGAUAACAUGACCUGGCUGCAGUGGGAUAAGGAGAUCAG
CAACUACACACAGAUCAUCUACGGCCUGCUGGAGGAGAGCCAGAAUCAGCAGGAGAAGAACGAGCAGGA
CCUGCUGGCCCUGGAUUGAUAA (SEQ ID NO: 122)
Membrane bound
BG505_MD39_Link14_gp140_PDGFR (amino acid, dna, rna)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGSHSGSGGSGSGGHAAVGIGAVSLGFLGAAGSTMGAAS
MTLTVQARNLLSGIVQQQSNLLRAPEPQQHLLKDTHWGIKQLQARVLAVEHYLRDQQLLGIWGCSGKLICCTNVP
WNSSWSNRNLSEIWDNMTWLQWDKEISNYTQIIYGLLEESQNQQEKNEQDLLALDGGGSGGSGGSGGSGGSG
GSNAVGQDTQEVIVVPHSLPFKVVVISAILALVVLTIISLIILIMLWQKKPR** (SEQ ID NO: 126)
atggactggacatggattctgttcctggtcgctgccgctacaagagtgcattccgccgaaaacctgtgggtcaccgtctactatggagtgcccg
tgtggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccg
ccatcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggca
gcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggctctcacagcggctccggcggctctg
gcagcggcggccacgccgcagtgggcatcggagccgtgtccctgggctttctgggagcagcaggctccacaatgggagcagcctctatgaccct
gacagtgcaggccaggaatctgctgagcggcatcgtgcagcagcagtccaacctgctgagagccccagagccccagcagcacctgctgaagga
cacccactggggcatcaagcagctgcaggccagggtgctggcagtggagcactatctgagagatcagcagctgctgggcatctggggctgtagc
ggcaagctgatctgctgtaccaatgtgccctggaactctagctggtctaatcgcaacctgagcgagatctgggacaatatgacctggctgcagtg
ggataaggagatctccaactacacacagatcatctatggcctgctggaagaatctcagaatcagcaggaaaagaatgaacaggatctgctggc
actggatggaggaggaagcgggggaagcgggggaagcggaggaagcgggggaagcgggggaagcaacgccgtgggccaggacacccagg
aagtgatcgtggtgccccacagcctgcctttcaaggtggtggtcatctccgccatcctggccctggtcgtgctgactattatttccctgatta
tcctgattatgctgtggcagaagaagcccagatgataa (SEQ ID NO: 124)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCUCUCACAGCGGCUCCGGCGGCUCUGGCAGCGGCGGCC
ACGCCGCAGUGGGCAUCGGAGCCGUGUCCCUGGGCUUUCUGGGAGCAGCAGGCUCCACAAUGGGAGCA
GCCUCUAUGACCCUGACAGUGCAGGCCAGGAAUCUGCUGAGCGGCAUCGUGCAGCAGCAGUCCAACCUG
CUGAGAGCCCCAGAGCCCCAGCAGCACCUGCUGAAGGACACCCACUGGGGCAUCAAGCAGCUGCAGGCCA
GGGUGCUGGCAGUGGAGCACUAUCUGAGAGAUCAGCAGCUGCUGGGCAUCUGGGGCUGUAGCGGCAAG
CUGAUCUGCUGUACCAAUGUGCCCUGGAACUCUAGCUGGUCUAAUCGCAACCUGAGCGAGAUCUGGGA
CAAUAUGACCUGGCUGCAGUGGGAUAAGGAGAUCUCCAACUACACACAGAUCAUCUAUGGCCUGCUGGA
AGAAUCUCAGAAUCAGCAGGAAAAGAAUGAACAGGAUCUGCUGGCACUGGAUGGAGGAGGAAGCGGGG
GAAGCGGGGGAAGCGGAGGAAGCGGGGGAAGCGGGGGAAGCAACGCCGUGGGCCAGGACACCCAGGAA
GUGAUCGUGGUGCCCCACAGCCUGCCUUUCAAGGUGGUGGUCAUCUCCGCCAUCCUGGCCCUGGUCGU
GCUGACUAUUAUUUCCCUGAUUAUCCUGAUUAUGCUGUGGCAGAAGAAGCCCAGAUGAUAA (SEQ ID
NO: 125)
BG505_MD39_Link14_gp140_Foldon-PDGFR (amino acid, dna, rna)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGSHSGSGGSGSGGHAAVGIGAVSLGFLGAAGSTMGAAS
MTLTVQARNLLSGIVQQQSNLLRAPEPQQHLLKDTHWGIKQLQARVLAVEHYLRDQQLLGIWGCSGKLICCTNVP
WNSSWSNRNLSEIWDNMTWLQWDKEISNYTQIIYGLLEESQNQQEKNEQDLLALDGGGSGGSGGGYIPEAPRD
GQAYVRKDGEWVLLSTFLGGSGGSGGSGGSNAVGQDTQEVIVVPHSLPFKVVVISAILALVVLTIISLIILIMLWQKK
PR** (SEQ ID NO: 129)
atggactggacatggattctgttcctggtcgctgccgctacaagagtgcattccgccgaaaacctgtgggtcaccgtctactatggagtgccc
gtgtggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccg
ccatcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggca
gcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggctctcacagcggctccggcggctctg
gcagcggcggccacgccgcagtgggcatcggagccgtgtccctgggctttctgggagcagcaggctccacaatgggagcagcctctatgaccct
gacagtgcaggccaggaatctgctgagcggcatcgtgcagcagcagtccaacctgctgagagccccagagccccagcagcacctgctgaagga
cacccactggggcatcaagcagctgcaggccagggtgctggcagtggagcactatctgagagatcagcagctgctgggcatctggggctgtagc
ggcaagctgatctgctgtaccaatgtgccctggaactctagctggtctaatcgcaacctgagcgagatctgggacaatatgacctggctgcagtg
ggataaggagatctccaactacacacagatcatctatggcctgctggaagaatctcagaatcagcaggaaaagaatgaacaggatctgctggc
actggatggaggaggaagcgggggaagcggcggcggctacatccctgaggccccaagggacggacaggcctatgtgagaaaggatggcgag
tgggtgctgctgtccaccttcctggggggaagcggaggaagcgggggaagcgggggaagcaacgccgtgggccaggacacccaggaagtgat
cgtggtgccccacagcctgcctttcaaggtggtggtcatctccgccatcctggccctggtcgtgctgactattatttccctgattatcctgatt
atgctgtggcagaagaagcccagatgataa (SEQ ID NO: 127)
BG505_MD39_trimer string 1 gp140_PDGFR (amino acid, dna, rna)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGSHSGSGGSGSGGHAAVGIGAVSLGFLGAAGSTMGAAS
MTLTVQARNLLSGIVQQQSNLLRAPEPQQHLLKDTHWGIKQLQARVLAVEHYLRDQQLLGIWGCSGKLICCTNVP
WNSSWSNRNLSEIWDNMTWLQWDKEISNYTQIIYGLLEESQNQQEKNEQDLLALDGGAENLWVTVYYGVPVW
KDAETTLFCASDAKAYETEKHNVWATHACVPTDPNPQEIHLENVTEEFNMWKNNMVEQMHEDIISLWDQSLKP
CVKLTPLCVTLQCTNVTNNITDDMRGELKNCSFNMTTELRDKKQKVYSLFYRLDVVQINENQGNRSNNSNKEYRLI
NCNTSAITQACPKVSFEPIPIHYCAPAGFAILKCKDKKFNGTGPCPSVSTVQCTHGIKPVVSTQLLLNGSLAEEEVIIRS
ENITNNAKNILVQLNTPVQINCTRPNNNTVKSIRIGPGQAFYYTGDIIGDIRQAHCNVSKATWNETLGKVVKQLRK
HFGNNTIIRFAQSSGGDLEVTTHSFNCGGEFFYCNTSGLFNSTWISNTSVQGSNSTGSNDSITLPCRIKQIINMWQR
IGQAMYAPPIQGVIRCVSNITGLILTRDGGSTNSTTETFRPGGGDMRDNWRSELYKYKVVKIEPLGVAPTRCKRRVV
GSHSGSGGSGSGGHAAVGIGAVSLGFLGAAGSTMGAASMTLTVQARNLLSGIVQQQSNLLRAPEPQQHLLKDTH
WGIKQLQARVLAVEHYLRDQQLLGIWGCSGKLICCTNVPWNSSWSNRNLSEIW/DNMTWLQWDKEISNYTQIIY
GLLEESQNQQEKNEQDLLALDGGAENLWVTVYYGVPVWKDAETTLFCASDAKAYETEKHNVWATHACVPTDPN
PQEIHLENVTEEFNMWKNNMVEQMHEDIISLWDQSLKPCVKLTPLCVTLQCTNVTNNITDDMRGELKNCSFNM
TTELRDKKQKVYSLFYRLDVVQINENQGNRSNNSNKEYRLINCNTSAITQACPKVSFEPIPIHYCAPAGFAILKCKDKK
FNGTGPCPSVSTVQCTHGIKPVVSTQLLLNGSLAEEEVIIRSENITNNAKNILVQLNTPVQINCTRPNNNTVKSIRIGP
GQAFYYTGDIIGDIRQAHCNVSKATWNETLGKVVKQLRKHFGNNTIIRFAQSSGGDLEVTTHSFNCGGEFFYCNTS
GLFNSTWISNTSVQGSNSTGSNDSITLPCRIKQIINMWQRIGQAMYAPPIQGVIRCVSNITGLILTRDGGSTNSTTET
FRPGGGDMRDNWRSELYKYKVVKIEPLGVAPTRCKRRVVGSHSGSGGSGSGGHAAVGIGAVSLGFLGAAGSTMG
AASMTLTVQARNLLSGIVQQQSNLLRAPEPQQHLLKDTHWGIKQLQARVLAVEHYLRDQQLLGIWGCSGKLICCT
NVPWNSSWSNRNLSEIWDNMTWLQWDKEISNYTQIIYGLLEESQNQQEKNEQDLLALDGGGSGGSGGSGGSG
GSGGSNAVGQDTQEVIVVPHSLPFKVVVISAILALVVLTIISLIILIMLWQKKPR** (SEQ ID NO: 132)
atggactggacatggattctgttcctggtcgctgccgctacaagagtgcattccgccgaaaacctgtgggtcaccgtctactatggagtgccc
gtgtggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccg
ccatcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggca
gcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggctctcacagcggctccggcggctctg
gcagcggcggccacgccgcagtgggcatcggagccgtgtccctgggctttctgggagcagcaggctccacaatgggagcagcctctatgaccct
gacagtgcaggccaggaatctgctgagcggcatcgtgcagcagcagtccaacctgctgagagccccagagccccagcagcacctgctgaagga
cacccactggggcatcaagcagctgcaggccagggtgctggcagtggagcactatctgagagatcagcagctgctgggcatctggggctgtagc
ggcaagctgatctgctgtaccaatgtgccctggaactctagctggtctaatcgcaacctgagcgagatctgggacaatatgacctggctgcagtg
ggataaggagatctccaactacacacagatcatctatggcctgctggaagaatctcagaatcagcaggaaaagaatgaacaggatctgctggc
actggatggcggcgccgaaaacctgtgggtcaccgtgtactacggagtccccgtgtggaaagatgcagagacaaccctgttctgcgcttccgac
gctaaagcttacgagacagaaaaacacaacgtgtgggccactcatgcctgcgtgcctacagaccctaacccacaggaaatccacctggagaat
gtgacggaggagtttaacatgtggaagaataacatggtcgagcagatgcatgaagatatcatttccttatgggaccaatccctgaagccttgcgt
gaagctgaccccactgtgcgtgacactgcaatgcactaacgtgaccaataacattaccgacgatatgcgcggcgagctgaagaactgctctttc
aacatgactaccgagctgagagataagaaacagaaagtgtacagcctgttttatcggttagatgtggtgcagatcaatgaaaaccagggcaat
cggtccaacaattctaacaaggaatatcgcctgatcaattgtaacacctccgccattacccaggcttgccctaaggtgtctttcgagcccatccct
atccactattgcgccccagctggatttgctatcctgaagtgtaaggacaaaaagtttaacgggaccggaccatgtcctagcgtgtccactgtgca
gtgcacccatggcatcaagcctgtggtgtccacccaacttctgctgaatggctctctggctgaagaagaagtgatcattaggtccgaaaatattac
taataacgctaaaaatatcctggtccagctgaacacgcctgtccagatcaattgtacccggccaaataacaacacagtgaagtctatcagaatc
ggcccaggccaggccttctactacacaggcgacattatcggcgatattcgccaggcccactgtaatgtgagcaaagctacatggaatgagacac
tgggcaaggtagtcaaacagctgagaaaacattttggaaacaacaccatcatccgctttgcacagtctagcggcggcgacctggaggtaactac
ccacagcttcaattgtggcggcgagttcttttactgtaataccagcggcctgtttaatagtacttggatcagcaacacatctgtgcagggctctaa
ctccactggctctaacgatagcatcacactgccttgtcggatcaagcaaatcatcaacatgtggcaaaggattgggcaggctatgtatgcccctcc
aatccagggcgtgatccggtgcgtgagcaacattacaggcctgatcctgacaagagacggcggctccaccaactctactaccgagacattccgg
cccggcggcggcgacatgcgtgataactggcgcagcgaactgtataaatataaagtggtgaagatcgagcctctgggcgtggccccaactaggt
gtaaaagaagggtcgtcggctcccacagcggcagcggcggctccggctctggcggccacgcggctgtcggcatcggcgccgtgagcctgggctt
tctgggcgccgccggctccactatgggcgcagcctctatgaccctgactgtccaggctagaaatctgctgtctggaatcgtgcagcagcagtcta
acctgctgagggcacctgagccacaacagcacctgctgaaggatacacattggggcatcaagcagttacaagccagggtgctggccgtggaac
actacctgcgcgatcagcaattactgggcatttggggatgctctggcaagctgatttgttgcaccaatgtgccctggaactcctcttggagcaac
agaaacctgtccgaaatctgggataacatgacatggctgcagtgggacaaggaaatttccaattatacccagatcatctatggactgctggaaga
aagtcagaatcagcaggagaagaatgaacaggatctgctggcactggatggcggcgccgaaaacctgtgggtcaccgtgtattatggagtgcc
agtgtggaaggacgccgagaccacactgttttgtgcctctgatgccaaggcctacgagaccgagaagcacaacgtgtgggccacccacgcctgc
gtgcccacagacccaaatcctcaggagatccacctggagaacgtgaccgaggagtttaacatgtggaagaacaatatggtggagcagatgcac
gaggatatcatctctctgtgggatcagtctctgaagccatgtgtgaagctgaccccactgtgcgtgaccctgcagtgtacaaatgtgacaaacaa
catcacagatgacatgagaggcgagctgaagaactgttccttcaatatgaccaccgagctgagagacaagaagcagaaggtgtattctctgttt
taccggctggacgtggtgcagatcaacgagaatcagggcaatcggtctaacaactccaataaggagtatagactgatcaactgcaacacctctg
ccatcacccaggcctgtcctaaggtgtcctttgagccaatcccaatccactattgcgcccctgccggctttgccatcctgaagtgcaaggacaaga
agtttaacggcacaggcccctgcccatccgtgagcacagtgcagtgtacccacggcatcaagcctgtggtgtccacccagctgctgctgaacggc
tccctggccgaggaggaggtaatcatcaggtctgagaacatcacaaataacgccaagaacatcctggtgcagctgaacaccccagtgcagatc
aactgtacccggcctaacaataataccgtgaagtctatccggatcggcccaggccaggccttctactataccggcgatatcatcggcgatatcag
acaggcccactgcaacgtgtccaaggccacatggaacgagacactgggcaaggtggtgaagcagctgcggaagcactttggcaataacacca
tcatcagattcgcccagtcttccggcggcgacctggaggtgacaacccactccttcaattgcggcggcgagttcttttactgtaatacaagcggcc
tgtttaatagcacctggatctctaacacctccgtgcagggctccaacagcacaggctctaatgattccatcaccctgccttgccggatcaagcaga
tcatcaatatgtggcagagaatcggccaggccatgtatgcccctccaatccagggcgtgatccgctgcgtgtccaacatcacaggcctgatcctg
acaagagatggcggctccaccaacagcaccacagagaccttcagacccggcggcggcgacatgcgcgacaactggagatccgagctgtataa
gtacaaggtggtgaagatcgagcccctgggcgtggccccaacccggtgtaagcgcagagtggtgggcagccacagcggcagcggcggcagcg
gctccggcggccacgccgccgtgggcatcggcgccgtgtccctgggcttcctgggcgccgccggctccaccatgggcgccgcctccatgacactg
acagtgcaggccagaaatctgctgtccggcatcgtgcagcagcagtccaatctgctgcgggcccctgagccacagcagcacctgctgaaggata
cccactggggcatcaagcagctgcaggcccgggtgctggccgtggagcactacctgagggatcagcagctgctgggcatctggggctgttccgg
caagctgatctgctgtacaaacgtgccctggaacagctcctggtccaataggaacctgtccgagatctgggataacatgacctggctgcagtggg
ataaggagatcagcaactacacacagatcatctacggcctgctggaggagagccagaatcagcaggagaagaacgagcaggacctgctggcc
ctggatggaggaggaagcgggggaagcgggggaagcggaggaagcgggggaagcgggggaagcaacgccgtgggccaggacacccagga
agtgatcgtggtgccccacagcctgcctttcaaggtggtggtcatctccgccatcctggccctggtcgtgctgactattatttccctgattat
cctgattatgctgtggcagaagaagcccagatgataa (SEQ ID NO: 130)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCUCUCACAGCGGCUCCGGCGGCUCUGGCAGCGGCGGCC
ACGCCGCAGUGGGCAUCGGAGCCGUGUCCCUGGGCUUUCUGGGAGCAGCAGGCUCCACAAUGGGAGCA
GCCUCUAUGACCCUGACAGUGCAGGCCAGGAAUCUGCUGAGCGGCAUCGUGCAGCAGCAGUCCAACCUG
CUGAGAGCCCCAGAGCCCCAGCAGCACCUGCUGAAGGACACCCACUGGGGCAUCAAGCAGCUGCAGGCCA
GGGUGCUGGCAGUGGAGCACUAUCUGAGAGAUCAGCAGCUGCUGGGCAUCUGGGGCUGUAGCGGCAAG
CUGAUCUGCUGUACCAAUGUGCCCUGGAACUCUAGCUGGUCUAAUCGCAACCUGAGCGAGAUCUGGGA
CAAUAUGACCUGGCUGCAGUGGGAUAAGGAGAUCUCCAACUACACACAGAUCAUCUAUGGCCUGCUGGA
AGAAUCUCAGAAUCAGCAGGAAAAGAAUGAACAGGAUCUGCUGGCACUGGAUGGCGGCGCCGAAAACCU
GUGGGUCACCGUGUACUACGGAGUCCCCGUGUGGAAAGAUGCAGAGACAACCCUGUUCUGCGCUUCCG
ACGCUAAAGCUUACGAGACAGAAAAACACAACGUGUGGGCCACUCAUGCCUGCGUGCCUACAGACCCUAA
CCCACAGGAAAUCCACCUGGAGAAUGUGACGGAGGAGUUUAACAUGUGGAAGAAUAACAUGGUCGAGC
AGAUGCAUGAAGAUAUCAUUUCCUUAUGGGACCAAUCCCUGAAGCCUUGCGUGAAGCUGACCCCACUGU
GCGUGACACUGCAAUGCACUAACGUGACCAAUAACAUUACCGACGAUAUGCGCGGCGAGCUGAAGAACU
GCUCUUUCAACAUGACUACCGAGCUGAGAGAUAAGAAACAGAAAGUGUACAGCCUGUUUUAUCGGUUA
GAUGUGGUGCAGAUCAAUGAAAACCAGGGCAAUCGGUCCAACAAUUCUAACAAGGAAUAUCGCCUGAUC
AAUUGUAACACCUCCGCCAUUACCCAGGCUUGCCCUAAGGUGUCUUUCGAGCCCAUCCCUAUCCACUAU
UGCGCCCCAGCUGGAUUUGCUAUCCUGAAGUGUAAGGACAAAAAGUUUAACGGGACCGGACCAUGUCC
UAGCGUGUCCACUGUGCAGUGCACCCAUGGCAUCAAGCCUGUGGUGUCCACCCAACUUCUGCUGAAUGG
CUCUCUGGCUGAAGAAGAAGUGAUCAUUAGGUCCGAAAAUAUUACUAAUAACGCUAAAAAUAUCCUGG
UCCAGCUGAACACGCCUGUCCAGAUCAAUUGUACCCGGCCAAAUAACAACACAGUGAAGUCUAUCAGAA
UCGGCCCAGGCCAGGCCUUCUACUACACAGGCGACAUUAUCGGCGAUAUUCGCCAGGCCCACUGUAAUG
UGAGCAAAGCUACAUGGAAUGAGACACUGGGCAAGGUAGUCAAACAGCUGAGAAAACAUUUUGGAAAC
AACACCAUCAUCCGCUUUGCACAGUCUAGCGGCGGCGACCUGGAGGUAACUACCCACAGCUUCAAUUGU
GGCGGCGAGUUCUUUUACUGUAAUACCAGCGGCCUGUUUAAUAGUACUUGGAUCAGCAACACAUCUGU
GCAGGGCUCUAACUCCACUGGCUCUAACGAUAGCAUCACACUGCCUUGUCGGAUCAAGCAAAUCAUCAA
CAUGUGGCAAAGGAUUGGGCAGGCUAUGUAUGCCCCUCCAAUCCAGGGCGUGAUCCGGUGCGUGAGCA
ACAUUACAGGCCUGAUCCUGACAAGAGACGGCGGCUCCACCAACUCUACUACCGAGACAUUCCGGCCCGG
CGGCGGCGACAUGCGUGAUAACUGGCGCAGCGAACUGUAUAAAUAUAAAGUGGUGAAGAUCGAGCCUC
UGGGCGUGGCCCCAACUAGGUGUAAAAGAAGGGUCGUCGGCUCCCACAGCGGCAGCGGCGGCUCCGGCU
CUGGCGGCCACGCGGCUGUCGGCAUCGGCGCCGUGAGCCUGGGCUUUCUGGGCGCCGCCGGCUCCACUA
UGGGCGCAGCCUCUAUGACCCUGACUGUCCAGGCUAGAAAUCUGCUGUCUGGAAUCGUGCAGCAGCAG
UCUAACCUGCUGAGGGCACCUGAGCCACAACAGCACCUGCUGAAGGAUACACAUUGGGGCAUCAAGCAG
UUACAAGCCAGGGUGCUGGCCGUGGAACACUACCUGCGCGAUCAGCAAUUACUGGGCAUUUGGGGAUG
CUCUGGCAAGCUGAUUUGUUGCACCAAUGUGCCCUGGAACUCCUCUUGGAGCAACAGAAACCUGUCCGA
AAUCUGGGAUAACAUGACAUGGCUGCAGUGGGACAAGGAAAUUUCCAAUUAUACCCAGAUCAUCUAUG
GACUGCUGGAAGAAAGUCAGAAUCAGCAGGAGAAGAAUGAACAGGAUCUGCUGGCACUGGAUGGCGGC
GCCGAAAACCUGUGGGUCACCGUGUAUUAUGGAGUGCCAGUGUGGAAGGACGCCGAGACCACACUGUU
UUGUGCCUCUGAUGCCAAGGCCUACGAGACCGAGAAGCACAACGUGUGGGCCACCCACGCCUGCGUGCC
CACAGACCCAAAUCCUCAGGAGAUCCACCUGGAGAACGUGACCGAGGAGUUUAACAUGUGGAAGAACAA
UAUGGUGGAGCAGAUGCACGAGGAUAUCAUCUCUCUGUGGGAUCAGUCUCUGAAGCCAUGUGUGAAGC
UGACCCCACUGUGCGUGACCCUGCAGUGUACAAAUGUGACAAACAACAUCACAGAUGACAUGAGAGGCG
AGCUGAAGAACUGUUCCUUCAAUAUGACCACCGAGCUGAGAGACAAGAAGCAGAAGGUGUAUUCUCUG
UUUUACCGGCUGGACGUGGUGCAGAUCAACGAGAAUCAGGGCAAUCGGUCUAACAACUCCAAUAAGGA
GUAUAGACUGAUCAACUGCAACACCUCUGCCAUCACCCAGGCCUGUCCUAAGGUGUCCUUUGAGCCAAU
CCCAAUCCACUAUUGCGCCCCUGCCGGCUUUGCCAUCCUGAAGUGCAAGGACAAGAAGUUUAACGGCAC
AGGCCCCUGCCCAUCCGUGAGCACAGUGCAGUGUACCCACGGCAUCAAGCCUGUGGUGUCCACCCAGCU
GCUGCUGAACGGCUCCCUGGCCGAGGAGGAGGUAAUCAUCAGGUCUGAGAACAUCACAAAUAACGCCAA
GAACAUCCUGGUGCAGCUGAACACCCCAGUGCAGAUCAACUGUACCCGGCCUAACAAUAAUACCGUGAA
GUCUAUCCGGAUCGGCCCAGGCCAGGCCUUCUACUAUACCGGCGAUAUCAUCGGCGAUAUCAGACAGGC
CCACUGCAACGUGUCCAAGGCCACAUGGAACGAGACACUGGGCAAGGUGGUGAAGCAGCUGCGGAAGCA
CUUUGGCAAUAACACCAUCAUCAGAUUCGCCCAGUCUUCCGGCGGCGACCUGGAGGUGACAACCCACUCC
UUCAAUUGCGGCGGCGAGUUCUUUUACUGUAAUACAAGCGGCCUGUUUAAUAGCACCUGGAUCUCUAA
CACCUCCGUGCAGGGCUCCAACAGCACAGGCUCUAAUGAUUCCAUCACCCUGCCUUGCCGGAUCAAGCAG
AUCAUCAAUAUGUGGCAGAGAAUCGGCCAGGCCAUGUAUGCCCCUCCAAUCCAGGGCGUGAUCCGCUGC
GUGUCCAACAUCACAGGCCUGAUCCUGACAAGAGAUGGCGGCUCCACCAACAGCACCACAGAGACCUUCA
GACCCGGCGGCGGCGACAUGCGCGACAACUGGAGAUCCGAGCUGUAUAAGUACAAGGUGGUGAAGAUC
GAGCCCCUGGGCGUGGCCCCAACCCGGUGUAAGCGCAGAGUGGUGGGCAGCCACAGCGGCAGCGGCGGC
AGCGGCUCCGGCGGCCACGCCGCCGUGGGCAUCGGCGCCGUGUCCCUGGGCUUCCUGGGCGCCGCCGGC
UCCACCAUGGGCGCCGCCUCCAUGACACUGACAGUGCAGGCCAGAAAUCUGCUGUCCGGCAUCGUGCAG
CAGCAGUCCAAUCUGCUGCGGGCCCCUGAGCCACAGCAGCACCUGCUGAAGGAUACCCACUGGGGCAUCA
AGCAGCUGCAGGCCCGGGUGCUGGCCGUGGAGCACUACCUGAGGGAUCAGCAGCUGCUGGGCAUCUGG
GGCUGUUCCGGCAAGCUGAUCUGCUGUACAAACGUGCCCUGGAACAGCUCCUGGUCCAAUAGGAACCUG
UCCGAGAUCUGGGAUAACAUGACCUGGCUGCAGUGGGAUAAGGAGAUCAGCAACUACACACAGAUCAUC
UACGGCCUGCUGGAGGAGAGCCAGAAUCAGCAGGAGAAGAACGAGCAGGACCUGCUGGCCCUGGAUGG
AGGAGGAAGCGGGGGAAGCGGGGGAAGCGGAGGAAGCGGGGGAAGCGGGGGAAGCAACGCCGUGGGCC
AGGACACCCAGGAAGUGAUCGUGGUGCCCCACAGCCUGCCUUUCAAGGUGGUGGUCAUCUCCGCCAUCC
UGGCCCUGGUCGUGCUGACUAUUAUUUCCCUGAUUAUCCUGAUUAUGCUGUGGCAGAAGAAGCCCAGA
UGAUAA (SEQ ID NO: 131)
Nanoparticles
BG505_MD39_3BVE (amino acid, dna, rna)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGRRRRRRAVGIGAVSLGFLGAAGSTMGAASMTLTVQAR
NLLSGIVQQQSNLLRAPEPQQHLLKDTHWGIKQLQARVLAVEHYLRDQQLLGIWGCSGKLICCTNVPWNSSWSN
RNLSEIWDNMTWLQWDKEISNYTQIIYGLLEESQNQQEKNEQDLLALDGSGGLSKDIIKLLNEQVNKEMQSSNLY
MSMSSWCYTHSLDGAGLFLFDHAAEEYEHAKKLIIFLNENNVPVQLTSISAPEHKFEGLTQIFQKAYEHEQHISESIN
NIVDHAIKSKDHATFNFLQWYVAEQHEEEVLFKDILDKIELIGNENHGLYLADQYVKGIAKSRKS** (SEQ ID
NO: 156)
atggactggacatggattctgttcctggtcgctgccgctacaagagtgcattccgccgaaaacctgtgggtcaccgtctactatggagtgccc
gtgtggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccgc
catcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggc
agcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggccggcgcaggagacggcgcgcagtg
ggcatcggagccgtgtccctgggctttctgggagcagcaggctccacaatgggagcagcctctatgaccctgacagtgcaggccaggaatctgc
tgagcggcatcgtgcagcagcagtccaacctgctgagagccccagagccccagcagcacctgctgaaggacacccactggggcatcaagcagc
tgcaggccagggtgctggcagtggagcactatctgagagatcagcagctgctgggcatctggggctgtagcggcaagctgatctgctgtaccaa
tgtgccctggaactctagctggtctaatcgcaacctgagcgagatctgggacaatatgacctggctgcagtgggataaggagatctccaactaca
cacagatcatctatggcctgctggaagaatctcagaatcagcaggaaaagaatgaacaggatctgctggcactggatggaagcggcgggctga
gtaaggacattatcaagctgctgaacgaacaggtgaacaaagagatgcagtctagcaacctgtacatgtccatgagctcctggtgctataccca
ctctctggacggagcaggcctgttcctgtttgatcacgccgccgaggagtacgagcacgccaagaagctgatcatcttcctgaatgagaacaatg
tgcccgtgcagctgacctctatcagcgcccctgagcacaagttcgagggcctgacacagatctttcagaaggcctacgagcacgagcagcacat
ctccgagtctatcaacaatatcgtggaccacgccatcaagtccaaggatcacgccacattcaactttctgcagtggtacgtggccgagcagcac
gaggaggaggtgctgtttaaggacatcctggataagatcgagctgatcggcaatgagaaccacgggctgtacctggcagatcagtatgtcaagg
gcatcgctaagtcaaggaaaagctgataa (SEQ ID NO: 154)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCCGGCGCAGGAGACGGCGCGCAGUGGGCAUCGGAGCC
GUGUCCCUGGGCUUUCUGGGAGCAGCAGGCUCCACAAUGGGAGCAGCCUCUAUGACCCUGACAGUGCA
GGCCAGGAAUCUGCUGAGCGGCAUCGUGCAGCAGCAGUCCAACCUGCUGAGAGCCCCAGAGCCCCAGCA
GCACCUGCUGAAGGACACCCACUGGGGCAUCAAGCAGCUGCAGGCCAGGGUGCUGGCAGUGGAGCACUA
UCUGAGAGAUCAGCAGCUGCUGGGCAUCUGGGGCUGUAGCGGCAAGCUGAUCUGCUGUACCAAUGUGC
CCUGGAACUCUAGCUGGUCUAAUCGCAACCUGAGCGAGAUCUGGGACAAUAUGACCUGGCUGCAGUGG
GAUAAGGAGAUCUCCAACUACACACAGAUCAUCUAUGGCCUGCUGGAAGAAUCUCAGAAUCAGCAGGAA
AAGAAUGAACAGGAUCUGCUGGCACUGGAUGGAAGCGGCGGGCUGAGUAAGGACAUUAUCAAGCUGCU
GAACGAACAGGUGAACAAAGAGAUGCAGUCUAGCAACCUGUACAUGUCCAUGAGCUCCUGGUGCUAUAC
CCACUCUCUGGACGGAGCAGGCCUGUUCCUGUUUGAUCACGCCGCCGAGGAGUACGAGCACGCCAAGAA
GCUGAUCAUCUUCCUGAAUGAGAACAAUGUGCCCGUGCAGCUGACCUCUAUCAGCGCCCCUGAGCACAA
GUUCGAGGGCCUGACACAGAUCUUUCAGAAGGCCUACGAGCACGAGCAGCACAUCUCCGAGUCUAUCAA
CAAUAUCGUGGACCACGCCAUCAAGUCCAAGGAUCACGCCACAUUCAACUUUCUGCAGUGGUACGUGGC
CGAGCAGCACGAGGAGGAGGUGCUGUUUAAGGACAUCCUGGAUAAGAUCGAGCUGAUCGGCAAUGAGA
ACCACGGGCUGUACCUGGCAGAUCAGUAUGUCAAGGGCAUCGCUAAGUCAAGGAAAAGCUGAUAA
(SEQ ID NO: 155)
BG505_MD39_I3_1 (amino acid, dna, rna)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGRRRRRRAVGIGAVSLGFLGAAGSTMGAASMTLTVQAR
NLLSGIVQQQSNLLRAPEPQQHLLKDTHWGIKQLQARVLAVEHYLRDQQLLGIWGCSGKLICCTNVPWNSSWSN
RNLSEIWDNMTWLQWDKEISNYTQIIYGLLEESQNQQEKNEQDLLALDGGSGSGGSGGMKMEELFKKHKIVAVL
RANSVEEAKKKALAVFLGGVHLIEITFTVPDADTVIKELSFLKEMGAIIGAGTVTSVEQCRKAVESGAEFIVSPHLDEEI
SQFCKEKGVFYMPGVMTPTELVKAMKLGHTILKLFPGEVVGPQFVKAMKGPFPNVKFVPTGGVNLDNVCEWFK
AGVLAVGVGSALVKGTPVEVAEKAKAFVEKIRGCTE** (SEQ ID NO: 159)
atggactggacatggattctgttcctggtcgctgccgctacaagagtgcattccgccgaaaacctgtgggtcaccgtctactatggagtgcccg
tgtggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccgcc
atcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatgg
cagcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggccggcgcaggagacggcgcgcagtg
ggcatcggagccgtgtccctgggctttctgggagcagcaggctccacaatgggagcagcctctatgaccctgacagtgcaggccaggaatctgc
tgagcggcatcgtgcagcagcagtccaacctgctgagagccccagagccccagcagcacctgctgaaggacacccactggggcatcaagcagc
tgcaggccagggtgctggcagtggagcactatctgagagatcagcagctgctgggcatctggggctgtagcggcaagctgatctgctgtaccaa
tgtgccctggaactctagctggtctaatcgcaacctgagcgagatctgggacaatatgacctggctgcagtgggataaggagatctccaactaca
cacagatcatctatggcctgctggaagaatctcagaatcagcaggaaaagaatgaacaggatctgctggcactggatggcggcagcggcagcg
gcgggagcggaggaatgaagatggaagaactgttcaagaagcacaagatcgtggccgtgctgagggccaactccgtggaggaggccaagaagaa
ggccctggccgtgttcctgggcggcgtgcacctgatcgagatcacctttacagtgcccgacgccgataccgtgatcaaggagctgtctttcct
gaaggagatgggagcaatcatcggagcaggaaccgtgacaagcgtggagcagtgcagaaaggccgtggagagcggcgccgagtttatcgtgt
cccctcacctggacgaggagatctctcagttctgtaaggagaagggcgtgttttacatgccaggcgtgatgacccccacagagctggtgaaggc
catgaagctgggccacacaatcctgaagctgttccctggcgaggtggtgggcccacagtttgtgaaggccatgaagggccccttccctaatgtga
agtttgtgcccaccggcggcgtgaacctggataacgtgtgcgagtggttcaaggcaggcgtgctggcagtgggcgtgggcagcgccctggtgaa
gggcacacccgtggaagtcgctgagaaggcaaaggcattcgtggaaaagattagggggtgtactgagtgataa (SEQ ID NO: 157)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCCGGCGCAGGAGACGGCGCGCAGUGGGCAUCGGAGCC
GUGUCCCUGGGCUUUCUGGGAGCAGCAGGCUCCACAAUGGGAGCAGCCUCUAUGACCCUGACAGUGCA
GGCCAGGAAUCUGCUGAGCGGCAUCGUGCAGCAGCAGUCCAACCUGCUGAGAGCCCCAGAGCCCCAGCA
GCACCUGCUGAAGGACACCCACUGGGGCAUCAAGCAGCUGCAGGCCAGGGUGCUGGCAGUGGAGCACUA
UCUGAGAGAUCAGCAGCUGCUGGGCAUCUGGGGCUGUAGCGGCAAGCUGAUCUGCUGUACCAAUGUGC
CCUGGAACUCUAGCUGGUCUAAUCGCAACCUGAGCGAGAUCUGGGACAAUAUGACCUGGCUGCAGUGG
GAUAAGGAGAUCUCCAACUACACACAGAUCAUCUAUGGCCUGCUGGAAGAAUCUCAGAAUCAGCAGGAA
AAGAAUGAACAGGAUCUGCUGGCACUGGAUGGCGGCAGCGGCAGCGGCGGGAGCGGAGGAAUGAAGAU
GGAAGAACUGUUCAAGAAGCACAAGAUCGUGGCCGUGCUGAGGGCCAACUCCGUGGAGGAGGCCAAGA
AGAAGGCCCUGGCCGUGUUCCUGGGCGGCGUGCACCUGAUCGAGAUCACCUUUACAGUGCCCGACGCCG
AUACCGUGAUCAAGGAGCUGUCUUUCCUGAAGGAGAUGGGAGCAAUCAUCGGAGCAGGAACCGUGACA
AGCGUGGAGCAGUGCAGAAAGGCCGUGGAGAGCGGCGCCGAGUUUAUCGUGUCCCCUCACCUGGACGA
GGAGAUCUCUCAGUUCUGUAAGGAGAAGGGCGUGUUUUACAUGCCAGGCGUGAUGACCCCCACAGAGC
UGGUGAAGGCCAUGAAGCUGGGCCACACAAUCCUGAAGCUGUUCCCUGGCGAGGUGGUGGGCCCACAG
UUUGUGAAGGCCAUGAAGGGCCCCUUCCCUAAUGUGAAGUUUGUGCCCACCGGCGGCGUGAACCUGGA
UAACGUGUGCGAGUGGUUCAAGGCAGGCGUGCUGGCAGUGGGCGUGGGCAGCGCCCUGGUGAAGGGC
ACACCCGUGGAAGUCGCUGAGAAGGCAAAGGCAUUCGUGGAAAAGAUUAGGGGGUGUACUGAGUGAUA
A (SEQ ID NO: 158)
BG505_MD39_I3_2 (amino acid, dna, rna)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGRRRRRRAVGIGAVSLGFLGAAGSTMGAASMTLTVQAR
NLLSGIVQQQSNLLRAPEPQQHLLKDTHWGIKQLQARVLAVEHYLRDQQLLGIWGCSGKLICCTNVPWNSSWSN
RNLSEIWDNMTWLQWDKEISNYTQIIYGLLEESQNQQEKNEQDLLALDGGSDMRKDAERRFDKFVEAAKNKFDK
FKAALRKGDIKEERRKDMKKLARKEAEQARRAVRNRLSELLSKINDMPITNDQKKLMSNDVLKFAAEAEKKIEALAA
DAEGGSGSMKMEELFKKHKIVAVLRANSVEEAKKKALAVFLGGVHLIEITFTVPDADTVIKELSFLKEMGAIIGAGTV
TSVEQCRKAVESGAEFIVSPHLDEEISQFCKEKGVFYMPGVMTPTELVKAMKLGHTILKLFPGEVVGPQFVKAMKG
PFPNVKFVPTGGVNLDNVCEWFKAGVLAVGVGSALVKGTPVEVAEKAKAFVEKIRGCTE** (SEQ ID NO: 162)
atggactggacatggattctgttcctggtcgctgccgctacaagagtgcattccgccgaaaacctgtgggtcaccgtctactatggagtgcccg
tgtggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccgc
catcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggc
agcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggccggcgcaggagacggcgcgcagtg
ggcatcggagccgtgtccctgggctttctgggagcagcaggctccacaatgggagcagcctctatgaccctgacagtgcaggccaggaatctgc
tgagcggcatcgtgcagcagcagtccaacctgctgagagccccagagccccagcagcacctgctgaaggacacccactggggcatcaagcagc
tgcaggccagggtgctggcagtggagcactatctgagagatcagcagctgctgggcatctggggctgtagcggcaagctgatctgctgtaccaa
tgtgccctggaactctagctggtctaatcgcaacctgagcgagatctgggacaatatgacctggctgcagtgggataaggagatctccaactaca
cacagatcatctatggcctgctggaagaatctcagaatcagcaggaaaagaatgaacaggatctgctggcactggatggagggagcgatatga
gaaaggacgccgagagacggtttgataagttcgtggaggctgctaagaataagtttgacaagtttaaggctgccctgcggaagggcgacatca
aggaggagaggagaaaggatatgaagaagctggcaaggaaggaggcagagcaggcaaggagggccgtgaggaacagactgagcgagctg
ctgtccaagatcaacgacatgcccatcaccaatgatcagaagaagctgatgtctaatgacgtgctgaagttcgccgcagaagccgaaaagaag
attgaagccctggcagcagacgccgaaggaggaagcgggagcatgaagatggaagaactgttcaagaagcacaagatcgtggccgtgctga
gggccaactccgtggaggaggccaagaagaaggccctggccgtgttcctgggcggcgtgcacctgatcgagatcacctttacagtgcccgacgc
cgataccgtgatcaaggagctgtctttcctgaaggagatgggagcaatcatcggagcaggaaccgtgacaagcgtggagcagtgcagaaagg
ccgtggagagcggcgccgagtttatcgtgtcccctcacctggacgaggagatctctcagttctgtaaggagaagggcgtgttttacatgccaggc
gtgatgacccccacagagctggtgaaggccatgaagctgggccacacaatcctgaagctgttccctggcgaggtggtgggcccacagtttgtga
aggccatgaagggccccttccctaatgtgaagtttgtgcccaccggcggcgtgaacctggataacgtgtgcgagtggttcaaggcaggcgtgctg
gcagtgggcgtgggcagcgccctggtgaagggcacacccgtggaagtcgctgagaaggcaaaggcattcgtggaaaagattagggggtgtac
tgagtgataa (SEQ ID NO: 160)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCCGGCGCAGGAGACGGCGCGCAGUGGGCAUCGGAGCC
GUGUCCCUGGGCUUUCUGGGAGCAGCAGGCUCCACAAUGGGAGCAGCCUCUAUGACCCUGACAGUGCA
GGCCAGGAAUCUGCUGAGCGGCAUCGUGCAGCAGCAGUCCAACCUGCUGAGAGCCCCAGAGCCCCAGCA
GCACCUGCUGAAGGACACCCACUGGGGCAUCAAGCAGCUGCAGGCCAGGGUGCUGGCAGUGGAGCACUA
UCUGAGAGAUCAGCAGCUGCUGGGCAUCUGGGGCUGUAGCGGCAAGCUGAUCUGCUGUACCAAUGUGC
CCUGGAACUCUAGCUGGUCUAAUCGCAACCUGAGCGAGAUCUGGGACAAUAUGACCUGGCUGCAGUGG
GAUAAGGAGAUCUCCAACUACACACAGAUCAUCUAUGGCCUGCUGGAAGAAUCUCAGAAUCAGCAGGAA
AAGAAUGAACAGGAUCUGCUGGCACUGGAUGGAGGGAGCGAUAUGAGAAAGGACGCCGAGAGACGGUU
UGAUAAGUUCGUGGAGGCUGCUAAGAAUAAGUUUGACAAGUUUAAGGCUGCCCUGCGGAAGGGCGACA
UCAAGGAGGAGAGGAGAAAGGAUAUGAAGAAGCUGGCAAGGAAGGAGGCAGAGCAGGCAAGGAGGGCC
GUGAGGAACAGACUGAGCGAGCUGCUGUCCAAGAUCAACGACAUGCCCAUCACCAAUGAUCAGAAGAAG
CUGAUGUCUAAUGACGUGCUGAAGUUCGCCGCAGAAGCCGAAAAGAAGAUUGAAGCCCUGGCAGCAGAC
GCCGAAGGAGGAAGCGGGAGCAUGAAGAUGGAAGAACUGUUCAAGAAGCACAAGAUCGUGGCCGUGCU
GAGGGCCAACUCCGUGGAGGAGGCCAAGAAGAAGGCCCUGGCCGUGUUCCUGGGCGGCGUGCACCUGA
UCGAGAUCACCUUUACAGUGCCCGACGCCGAUACCGUGAUCAAGGAGCUGUCUUUCCUGAAGGAGAUG
GGAGCAAUCAUCGGAGCAGGAACCGUGACAAGCGUGGAGCAGUGCAGAAAGGCCGUGGAGAGCGGCGCC
GAGUUUAUCGUGUCCCCUCACCUGGACGAGGAGAUCUCUCAGUUCUGUAAGGAGAAGGGCGUGUUUUA
CAUGCCAGGCGUGAUGACCCCCACAGAGCUGGUGAAGGCCAUGAAGCUGGGCCACACAAUCCUGAAGCU
GUUCCCUGGCGAGGUGGUGGGCCCACAGUUUGUGAAGGCCAUGAAGGGCCCCUUCCCUAAUGUGAAGU
UUGUGCCCACCGGCGGCGUGAACCUGGAUAACGUGUGCGAGUGGUUCAAGGCAGGCGUGCUGGCAGUG
GGCGUGGGCAGCGCCCUGGUGAAGGGCACACCCGUGGAAGUCGCUGAGAAGGCAAAGGCAUUCGUGGA
AAAGAUUAGGGGGUGUACUGAGUGAUAA (SEQ ID NO: 161)
BG505_MD39_LS_3CBPIX_1 (amino acid, dna, rna)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGRRRRRRAVGIGAVSLGFLGAAGSTMGAASMTLTVQAR
NLLSGIVQQQSNLLRAPEPQQHLLKDTHWGIKQLQARVLAVEHYLRDQQLLGIWGCSGKLICCTNVPWNSSWSN
RNLSEIWDNMTWLQWDKEISNYTQIIYGLLEESQNQQEKNEQDLLALDGGSSGKSLVDTVYALKDEVQELRQDNK

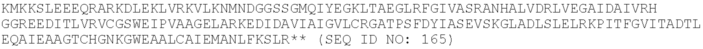

atggactggacatggattctgttcctggtcgctgccgctacaagagtgcattccgccgaaaacctgtgggtcaccgtctactatggagtgcccg
tgtggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccgcc
atcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatgg
cagcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggccggcgcaggagacggcgcgcagtg
ggcatcggagccgtgtccctgggctttctgggagcagcaggctccacaatgggagcagcctctatgaccctgacagtgcaggccaggaatctgc
tgagcggcatcgtgcagcagcagtccaacctgctgagagccccagagccccagcagcacctgctgaaggacacccactggggcatcaagcagc
tgcaggccagggtgctggcagtggagcactatctgagagatcagcagctgctgggcatctggggctgtagcggcaagctgatctgctgtaccaa
tgtgccctggaactctagctggtctaatcgcaacctgagcgagatctgggacaatatgacctggctgcagtgggataaggagatctccaactaca
cacagatcatctatggcctgctggaagaatctcagaatcagcaggaaaagaatgaacaggatctgctggcactggatgggggctctagcggga
aaagtctggtggataccgtctatgctctgaaagatgaggtgcaggaactgaggcaggacaacaaaaagatgaagaagagcctggaggagga
gcagagggccagaaaggacctggaaaaactggtgcggaaagtgctgaaaaacatgaatgacggagggagtagcgggatgcagatctacgaa
ggaaaactgaccgctgagggactgaggttcggaattgtcgcaagccgcgcgaatcacgcactggtggataggctggtggaaggcgctatcgac
gcaattgtccggcacggcgggagagaggaagacatcacactggtgagagtctgcggcagctgggagattcccgtggcagctggagaactggct
cgaaaggaggacatcgatgccgtgatcgctattggggtcctgtgccgaggagcaactcccagcttcgactacatcgcctcagaagtgagcaagg
ggctggctgatctgtccctggagctgaggaaacctatcacttttggcgtgattactgccgacaccctggaacaggcaatcgaggcggccggcacc
tgccatggaaacaaaggctgggaagcagccctgtgcgctattgagatggcaaatctgttcaaatctctgcgatgataa (SEQ ID NO: 163)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCCGGCGCAGGAGACGGCGCGCAGUGGGCAUCGGAGCC
GUGUCCCUGGGCUUUCUGGGAGCAGCAGGCUCCACAAUGGGAGCAGCCUCUAUGACCCUGACAGUGCA
GGCCAGGAAUCUGCUGAGCGGCAUCGUGCAGCAGCAGUCCAACCUGCUGAGAGCCCCAGAGCCCCAGCA
GCACCUGCUGAAGGACACCCACUGGGGCAUCAAGCAGCUGCAGGCCAGGGUGCUGGCAGUGGAGCACUA
UCUGAGAGAUCAGCAGCUGCUGGGCAUCUGGGGCUGUAGCGGCAAGCUGAUCUGCUGUACCAAUGUGC
CCUGGAACUCUAGCUGGUCUAAUCGCAACCUGAGCGAGAUCUGGGACAAUAUGACCUGGCUGCAGUGG
GAUAAGGAGAUCUCCAACUACACACAGAUCAUCUAUGGCCUGCUGGAAGAAUCUCAGAAUCAGCAGGAA
AAGAAUGAACAGGAUCUGCUGGCACUGGAUGGGGGCUCUAGCGGGAAAAGUCUGGUGGAUACCGUCUA
UGCUCUGAAAGAUGAGGUGCAGGAACUGAGGCAGGACAACAAAAAGAUGAAGAAGAGCCUGGAGGAGG
AGCAGAGGGCCAGAAAGGACCUGGAAAAACUGGUGCGGAAAGUGCUGAAAAACAUGAAUGACGGAGGG
AGUAGCGGGAUGCAGAUCUACGAAGGAAAACUGACCGCUGAGGGACUGAGGUUCGGAAUUGUCGCAAG
CCGCGCGAAUCACGCACUGGUGGAUAGGCUGGUGGAAGGCGCUAUCGACGCAAUUGUCCGGCACGGCG
GGAGAGAGGAAGACAUCACACUGGUGAGAGUCUGCGGCAGCUGGGAGAUUCCCGUGGCAGCUGGAGAA
CUGGCUCGAAAGGAGGACAUCGAUGCCGUGAUCGCUAUUGGGGUCCUGUGCCGAGGAGCAACUCCCAG
CUUCGACUACAUCGCCUCAGAAGUGAGCAAGGGGCUGGCUGAUCUGUCCCUGGAGCUGAGGAAACCUA
UCACUUUUGGCGUGAUUACUGCCGACACCCUGGAACAGGCAAUCGAGGCGGCCGGCACCUGCCAUGGAA
ACAAAGGCUGGGAAGCAGCCCUGUGCGCUAUUGAGAUGGCAAAUCUGUUCAAAUCUCUGCGAUGAUAA
(SEQ ID NO: 164)
BG505_MD39_LS_3CBPIX_2 (amino acid, dna, rna)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGRRRRRRAVGIGAVSLGFLGAAGSTMGAASMTLTVQAR
NLLSGIVQQQSNLLRAPEPQQHLLKDTHWGIKQLQARVLAVEHYLRDQQLLGIWGCSGKLICCTNVPWNSSWSN
RNLSEIWDNMTWLQWDKEISNYTQIIYGLLEESQNQQEKNEQDLLALDGGSSGADPKKVLDKAKDQAENRVREL
KQKLEELYKEARKLDLTQEMRRKLELRYIAAMLMAIGDIYNAIRQAKQEADKLKKAGLVNSQQLDELKRRLEELKEE
ASRKARDYGREFQLKLEYGGGSGSGSGMQIYEGKLTAEGLRFGIVASRANHALVDRLVEGAIDAIVRHGGREEDITL
VRVCGSWEIPVAAGELARKEDIDAVIAIGVLCRGATPSFDYIASEVSKGLADLSLELRKPITFGVITADTLEQAIEAAGT
CHGNKGWEAALCAIEMANLFKSLR** (SEQ ID NO: 168)
atggactggacatggattctgttcctggtcgctgccgctacaagagtgcattccgccgaaaacctgtgggtcaccgtctactatggagtgccc
gtgtggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccgc
catcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggc
agcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggccggcgcaggagacggcgcgcagtg
ggcatcggagccgtgtccctgggctttctgggagcagcaggctccacaatgggagcagcctctatgaccctgacagtgcaggccaggaatctgc
tgagcggcatcgtgcagcagcagtccaacctgctgagagccccagagccccagcagcacctgctgaaggacacccactggggcatcaagcagc
tgcaggccagggtgctggcagtggagcactatctgagagatcagcagctgctgggcatctggggctgtagcggcaagctgatctgctgtaccaa
tgtgccctggaactctagctggtctaatcgcaacctgagcgagatctgggacaatatgacctggctgcagtgggataaggagatctccaactaca
cacagatcatctatggcctgctggaagaatctcagaatcagcaggaaaagaatgaacaggatctgctggcactggatgggggctctagcgggg
cagacccaaagaaagtgctggataaggcaaaggatcaggcagagaatagagtgagagaactgaaacagaaactggaggaactgtataagg
aggcccggaagctggacctgacccaggagatgaggagaaagctggagctgcgctacatcgccgccatgctgatggccatcggcgacatctata
acgccatcaggcaggccaagcaggaggccgataagctgaagaaggccggcctggtgaatagccagcagctggacgagctgaagcggcgcct
ggaggagctgaaggaggaggcctccaggaaggccagagattatgggcgggaatttcagctgaaactggagtatggcggcggaagcggaagc
gggagcgggatgcagatctacgaaggaaaactgaccgctgagggactgaggttcggaattgtcgcaagccgcgcgaatcacgcactggtggat
aggctggtggaaggcgctatcgacgcaattgtccggcacggcgggagagaggaagacatcacactggtgagagtctgcggcagctgggagatt
cccgtggcagctggagaactggctcgaaaggaggacatcgatgccgtgatcgctattggggtcctgtgccgaggagcaactcccagcttcgact
acatcgcctcagaagtgagcaaggggctggctgatctgtccctggagctgaggaaacctatcacttttggcgtgattactgccgacaccctggaa
caggcaatcgaggcggccggcacctgccatggaaacaaaggctgggaagcagccctgtgcgctattgagatggcaaatctgttcaaatctctgc
gatgataa (SEQ ID NO: 166)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCCGGCGCAGGAGACGGCGCGCAGUGGGCAUCGGAGCC
GUGUCCCUGGGCUUUCUGGGAGCAGCAGGCUCCACAAUGGGAGCAGCCUCUAUGACCCUGACAGUGCA
GGCCAGGAAUCUGCUGAGCGGCAUCGUGCAGCAGCAGUCCAACCUGCUGAGAGCCCCAGAGCCCCAGCA
GCACCUGCUGAAGGACACCCACUGGGGCAUCAAGCAGCUGCAGGCCAGGGUGCUGGCAGUGGAGCACUA
UCUGAGAGAUCAGCAGCUGCUGGGCAUCUGGGGCUGUAGCGGCAAGCUGAUCUGCUGUACCAAUGUGC
CCUGGAACUCUAGCUGGUCUAAUCGCAACCUGAGCGAGAUCUGGGACAAUAUGACCUGGCUGCAGUGG
GAUAAGGAGAUCUCCAACUACACACAGAUCAUCUAUGGCCUGCUGGAAGAAUCUCAGAAUCAGCAGGAA
AAGAAUGAACAGGAUCUGCUGGCACUGGAUGGGGGCUCUAGCGGGGCAGACCCAAAGAAAGUGCUGGA
UAAGGCAAAGGAUCAGGCAGAGAAUAGAGUGAGAGAACUGAAACAGAAACUGGAGGAACUGUAUAAGG
AGGCCCGGAAGCUGGACCUGACCCAGGAGAUGAGGAGAAAGCUGGAGCUGCGCUACAUCGCCGCCAUGC
UGAUGGCCAUCGGCGACAUCUAUAACGCCAUCAGGCAGGCCAAGCAGGAGGCCGAUAAGCUGAAGAAGG
CCGGCCUGGUGAAUAGCCAGCAGCUGGACGAGCUGAAGCGGCGCCUGGAGGAGCUGAAGGAGGAGGCC
UCCAGGAAGGCCAGAGAUUAUGGGCGGGAAUUUCAGCUGAAACUGGAGUAUGGCGGCGGAAGCGGAAG
CGGGAGCGGGAUGCAGAUCUACGAAGGAAAACUGACCGCUGAGGGACUGAGGUUCGGAAUUGUCGCAA
GCCGCGCGAAUCACGCACUGGUGGAUAGGCUGGUGGAAGGCGCUAUCGACGCAAUUGUCCGGCACGGC
GGGAGAGAGGAAGACAUCACACUGGUGAGAGUCUGCGGCAGCUGGGAGAUUCCCGUGGCAGCUGGAGA
ACUGGCUCGAAAGGAGGACAUCGAUGCCGUGAUCGCUAUUGGGGUCCUGUGCCGAGGAGCAACUCCCA
GCUUCGACUACAUCGCCUCAGAAGUGAGCAAGGGGCUGGCUGAUCUGUCCCUGGAGCUGAGGAAACCU
AUCACUUUUGGCGUGAUUACUGCCGACACCCUGGAACAGGCAAUCGAGGCGGCCGGCACCUGCCAUGGA
AACAAAGGCUGGGAAGCAGCCCUGUGCGCUAUUGAGAUGGCAAAUCUGUUCAAAUCUCUGCGAUGAUA
A (SEQ ID NO: 167)
BG505_MD39_QB_1 (amino acid, dna, rna)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGRRRRRRAVGIGAVSLGFLGAAGSTMGAASMTLTVQAR
NLLSGIVQQQSNLLRAPEPQQHLLKDTHWGIKQLQARVLAVEHYLRDQQLLGIWGCSGKLICCTNVPWNSSWSN
RNLSEIWDNMTWLQWDKEISNYTQIIYGLLEESQNQQEKNEQDLLALDGGSSGGTDVGAIAGKANEAGQGAYDA
QVKNDEQDVELADHEARIKQLRIDVDDHESRITANTKAITALNVRVTTAEGEIASLQTNVSALDGRVTTAENNISAL
QADYVSGGSSGSGAKLETVTLGNIGKDGKQTLVLNPRGVNPTNGVASLSQAGAVPALEKRVTVSVSQPSRNRKNY
KVQVKIQNPTACTANGSCDPSVTRQAYADVTFSFTQYSTDEERAFVRTELAALLASPLLIDAIDQLNPAY**
(SEQ ID NO: 171)
atggactggacatggattctgttcctggtcgctgccgctacaagagtgcattccgccgaaaacctgtgggtcaccgtctactatggagtgcccg
tgtggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccgc
catcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggc
agcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggccggcgcaggagacggcgcgcagtg
ggcatcggagccgtgtccctgggctttctgggagcagcaggctccacaatgggagcagcctctatgaccctgacagtgcaggccaggaatctgc
tgagcggcatcgtgcagcagcagtccaacctgctgagagccccagagccccagcagcacctgctgaaggacacccactggggcatcaagcagc
tgcaggccagggtgctggcagtggagcactatctgagagatcagcagctgctgggcatctggggctgtagcggcaagctgatctgctgtaccaa
tgtgccctggaactctagctggtctaatcgcaacctgagcgagatctgggacaatatgacctggctgcagtgggataaggagatctccaactaca
cacagatcatctatggcctgctggaagaatctcagaatcagcaggaaaagaatgaacaggatctgctggcactggatggaggctcttcaggcg
gcacagacgtgggggcaatcgctggaaaggctaacgaggctggacagggggcttatgatgctcaggtcaaaaacgacgagcaggatgtggag
ctggccgaccacgaggccaggatcaagcagctgagaatcgatgtggacgatcacgagtctcggatcaccgccaacacaaaggccatcacagc
cctgaatgtgcgcgtgaccacagcagagggagagatcgcatccctgcagaccaacgtgagcgccctggacggaagggtgaccacagcagaga
acaatatctccgccctgcaggcagattacgtgagcggcggcagctccggctccggagcaaagctggagacagtgacactgggcaacatcggca
aggacggcaagcagacactggtgctgaatcccaggggcgtgaaccctaccaatggagtggcatctctgagccaggcaggagcagtgcctgccc
tggagaagagagtgaccgtgtccgtgtctcagcccagcaggaacagaaagaattataaggtgcaggtgaagatccagaacccaaccgcctgc
acagccaatggcagctgtgacccatccgtgacaaggcaggcatacgcagatgtgaccttctcttttacacagtatagcaccgatgaggagaggg
ccttcgtgcgcaccgagctggccgccctgctggcatcccctctgctgattgacgctattgaccagctgaaccctgcttactgataa (SEQ ID
NO: 169)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCCGGCGCAGGAGACGGCGCGCAGUGGGCAUCGGAGCC
GUGUCCCUGGGCUUUCUGGGAGCAGCAGGCUCCACAAUGGGAGCAGCCUCUAUGACCCUGACAGUGCA
GGCCAGGAAUCUGCUGAGCGGCAUCGUGCAGCAGCAGUCCAACCUGCUGAGAGCCCCAGAGCCCCAGCA
GCACCUGCUGAAGGACACCCACUGGGGCAUCAAGCAGCUGCAGGCCAGGGUGCUGGCAGUGGAGCACUA
UCUGAGAGAUCAGCAGCUGCUGGGCAUCUGGGGCUGUAGCGGCAAGCUGAUCUGCUGUACCAAUGUGC
CCUGGAACUCUAGCUGGUCUAAUCGCAACCUGAGCGAGAUCUGGGACAAUAUGACCUGGCUGCAGUGG
GAUAAGGAGAUCUCCAACUACACACAGAUCAUCUAUGGCCUGCUGGAAGAAUCUCAGAAUCAGCAGGAA
AAGAAUGAACAGGAUCUGCUGGCACUGGAUGGAGGCUCUUCAGGCGGCACAGACGUGGGGGCAAUCGC
UGGAAAGGCUAACGAGGCUGGACAGGGGGCUUAUGAUGCUCAGGUCAAAAACGACGAGCAGGAUGUGG
AGCUGGCCGACCACGAGGCCAGGAUCAAGCAGCUGAGAAUCGAUGUGGACGAUCACGAGUCUCGGAUCA
CCGCCAACACAAAGGCCAUCACAGCCCUGAAUGUGCGCGUGACCACAGCAGAGGGAGAGAUCGCAUCCCU
GCAGACCAACGUGAGCGCCCUGGACGGAAGGGUGACCACAGCAGAGAACAAUAUCUCCGCCCUGCAGGC
AGAUUACGUGAGCGGCGGCAGCUCCGGCUCCGGAGCAAAGCUGGAGACAGUGACACUGGGCAACAUCGG
CAAGGACGGCAAGCAGACACUGGUGCUGAAUCCCAGGGGCGUGAACCCUACCAAUGGAGUGGCAUCUCU
GAGCCAGGCAGGAGCAGUGCCUGCCCUGGAGAAGAGAGUGACCGUGUCCGUGUCUCAGCCCAGCAGGAA
CAGAAAGAAUUAUAAGGUGCAGGUGAAGAUCCAGAACCCAACCGCCUGCACAGCCAAUGGCAGCUGUGA
CCCAUCCGUGACAAGGCAGGCAUACGCAGAUGUGACCUUCUCUUUUACACAGUAUAGCACCGAUGAGGA
GAGGGCCUUCGUGCGCACCGAGCUGGCCGCCCUGCUGGCAUCCCCUCUGCUGAUUGACGCUAUUGACCA
GCUGAACCCUGCUUACUGAUAA (SEQ ID NO: 170)
BG505_MD39_QB_2 (amino acid, dna, rna)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGRRRRRRAVGIGAVSLGFLGAAGSTMGAASMTLTVQAR
NLLSGIVQQQSNLLRAPEPQQHLLKDTHWGIKQLQARVLAVEHYLRDQQLLGIWGCSGKLICCTNVPWNSSWSN
RNLSEIWDNMTWLQWDKEISNYTQIIYGLLEESQNQQEKNEQDLLALDGGSGSGGSSGPHMIAPGHRDEFDPKL
PTGEKEEVPGKPGIKNPETGDVVRPPVDSVTKYGPVKGDSIVEKEEIPFEKERKFNPDLAPGTEKVTREGQKGEKTIT
TPTLKNPLTGEIISKGESKEEITKDPINELTEWGPETGGSGSGGSSAKLETVTLGNIGKDGKQTLVLNPRGVNPTNGV
ASLSQAGAVPALEKRVTVSVSQPSRNRKNYKVQVKIQNPTACTANGSCDPSVTRQAYADVTFSFTQYSTDEERAFV
RTELAALLASPLLIDAIDQLNPAY** (SEQ ID NO: 174)
atggactggacatggattctgttcctggtcgctgccgctacaagagtgcattccgccgaaaacctgtgggtcaccgtctactatggagtgccc
gtgtggaaggacgccgagactacgctgttctgcgccagcgatgccaaggcctacgagacagagaagcacaacgtgtgggcaacccacgcatgcg
tgcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccgc
catcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggc
agcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggccggcgcaggagacggcgcgcagtg
ggcatcggagccgtgtccctgggctttctgggagcagcaggctccacaatgggagcagcctctatgaccctgacagtgcaggccaggaatctgc
tgagcggcatcgtgcagcagcagtccaacctgctgagagccccagagccccagcagcacctgctgaaggacacccactggggcatcaagcagc
tgcaggccagggtgctggcagtggagcactatctgagagatcagcagctgctgggcatctggggctgtagcggcaagctgatctgctgtaccaa
tgtgccctggaactctagctggtctaatcgcaacctgagcgagatctgggacaatatgacctggctgcagtgggataaggagatctccaactaca
cacagatcatctatggcctgctggaagaatctcagaatcagcaggaaaagaatgaacaggatctgctggcactggatggaggctctggaagcg
ggggaagtagcggacctcacatgattgctccaggacatcgggacgagtttgaccctaagctgccaacaggcgagaaagaagaggtgccaggc
aagcccggcatcaagaaccctgagacaggcgacgtggtgaggccccctgtggattctgtgacaaagtacggcccagtgaagggcgacagcatc
gtggagaaggaggagatccccttcgagaaggagaggaagtttaaccctgatctggccccaggcaccgagaaggtgacaagagagggccaga
agggcgagaagaccatcaccacacccacactgaagaatcctctgaccggcgagatcatcagcaagggcgagtccaaggaggagatcacaaa
ggaccccatcaacgaactgaccgaatggggaccagagacaggaggaagcggcagcggcggaagcagcgcaaagctggagacagtgacact
gggcaacatcggcaaggacggcaagcagacactggtgctgaatcccaggggcgtgaaccctaccaatggagtggcatctctgagccaggcag
gagcagtgcctgccctggagaagagagtgaccgtgtccgtgtctcagcccagcaggaacagaaagaattataaggtgcaggtgaagatccaga
acccaaccgcctgcacagccaatggcagctgtgacccatccgtgacaaggcaggcatacgcagatgtgaccttctcttttacacagtatagcacc
gatgaggagagggccttcgtgcgcaccgagctggccgccctgctggcatcccctctgctgattgacgctattgaccagctgaaccctgcttac
tgataa (SEQ ID NO: 172)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCCGGCGCAGGAGACGGCGCGCAGUGGGCAUCGGAGCC
GUGUCCCUGGGCUUUCUGGGAGCAGCAGGCUCCACAAUGGGAGCAGCCUCUAUGACCCUGACAGUGCA
GGCCAGGAAUCUGCUGAGCGGCAUCGUGCAGCAGCAGUCCAACCUGCUGAGAGCCCCAGAGCCCCAGCA
GCACCUGCUGAAGGACACCCACUGGGGCAUCAAGCAGCUGCAGGCCAGGGUGCUGGCAGUGGAGCACUA
UCUGAGAGAUCAGCAGCUGCUGGGCAUCUGGGGCUGUAGCGGCAAGCUGAUCUGCUGUACCAAUGUGC
CCUGGAACUCUAGCUGGUCUAAUCGCAACCUGAGCGAGAUCUGGGACAAUAUGACCUGGCUGCAGUGG
GAUAAGGAGAUCUCCAACUACACACAGAUCAUCUAUGGCCUGCUGGAAGAAUCUCAGAAUCAGCAGGAA
AAGAAUGAACAGGAUCUGCUGGCACUGGAUGGAGGCUCUGGAAGCGGGGGAAGUAGCGGACCUCACAU
GAUUGCUCCAGGACAUCGGGACGAGUUUGACCCUAAGCUGCCAACAGGCGAGAAAGAAGAGGUGCCAGG
CAAGCCCGGCAUCAAGAACCCUGAGACAGGCGACGUGGUGAGGCCCCCUGUGGAUUCUGUGACAAAGUA
CGGCCCAGUGAAGGGCGACAGCAUCGUGGAGAAGGAGGAGAUCCCCUUCGAGAAGGAGAGGAAGUUUA
ACCCUGAUCUGGCCCCAGGCACCGAGAAGGUGACAAGAGAGGGCCAGAAGGGCGAGAAGACCAUCACCAC
ACCCACACUGAAGAAUCCUCUGACCGGCGAGAUCAUCAGCAAGGGCGAGUCCAAGGAGGAGAUCACAAA
GGACCCCAUCAACGAACUGACCGAAUGGGGACCAGAGACAGGAGGAAGCGGCAGCGGCGGAAGCAGCGC
AAAGCUGGAGACAGUGACACUGGGCAACAUCGGCAAGGACGGCAAGCAGACACUGGUGCUGAAUCCCAG
GGGCGUGAACCCUACCAAUGGAGUGGCAUCUCUGAGCCAGGCAGGAGCAGUGCCUGCCCUGGAGAAGA
GAGUGACCGUGUCCGUGUCUCAGCCCAGCAGGAACAGAAAGAAUUAUAAGGUGCAGGUGAAGAUCCAG
AACCCAACCGCCUGCACAGCCAAUGGCAGCUGUGACCCAUCCGUGACAAGGCAGGCAUACGCAGAUGUGA
CCUUCUCUUUUACACAGUAUAGCACCGAUGAGGAGAGGGCCUUCGUGCGCACCGAGCUGGCCGCCCUGC
UGGCAUCCCCUCUGCUGAUUGACGCUAUUGACCAGCUGAACCCUGCUUACUGAUAA (SEQ ID NO: 173)
BG505_MD39_IC1 (amino acid, dna, rna)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGRRRRRRAVGIGAVSLGFLGAAGSTMGAASMTLTVQAR
NLLSGIVQQQSNLLRAPEPQQHLLKDTHWGIKQLQARVLAVEHYLRDQQLLGIWGCSGKLICCTNVPWNSSWSN
RNLSEIWDNMTWLQWDKEISNYTQIIYGLLEESQNQQEKNEQDLLALDGGSGSGSGDPEFTKNALNVVKNDLIAK
VDQLSGEQEVLRGELEAAKQAKVKLENRIKELEEELKRV** (SEQ ID NO: 177)
atggactggacatggattctgttcctggtcgctgccgctacaagagtgcattccgccgaaaacctgtgggtcaccgtctactatggagtgcccg
tgtggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacga
ggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccgcc
atcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggc
agcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggccggcgcaggagacggcgcgcagtg
ggcatcggagccgtgtccctgggctttctgggagcagcaggctccacaatgggagcagcctctatgaccctgacagtgcaggccaggaatctgc
tgagcggcatcgtgcagcagcagtccaacctgctgagagccccagagccccagcagcacctgctgaaggacacccactggggcatcaagcagc
tgcaggccagggtgctggcagtggagcactatctgagagatcagcagctgctgggcatctggggctgtagcggcaagctgatctgctgtaccaa
tgtgccctggaactctagctggtctaatcgcaacctgagcgagatctgggacaatatgacctggctgcagtgggataaggagatctccaactaca
cacagatcatctatggcctgctggaagaatctcagaatcagcaggaaaagaatgaacaggatctgctggcactggatggaggcagcggcagcg
gcagcggggaccctgagtttaccaaaaatgctctgaatgtcgtcaaaaatgatctgattgctaaggtggaccagctgagcggagagcaggaggt
gctgaggggcgagctggaggccgccaagcaggcaaaggtgaaactggaaaaccgaatcaaggaactggaagaagaactgaaaagagtctg
ataa (SEQ ID NO: 175)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCCGGCGCAGGAGACGGCGCGCAGUGGGCAUCGGAGCC
GUGUCCCUGGGCUUUCUGGGAGCAGCAGGCUCCACAAUGGGAGCAGCCUCUAUGACCCUGACAGUGCA
GGCCAGGAAUCUGCUGAGCGGCAUCGUGCAGCAGCAGUCCAACCUGCUGAGAGCCCCAGAGCCCCAGCA
GCACCUGCUGAAGGACACCCACUGGGGCAUCAAGCAGCUGCAGGCCAGGGUGCUGGCAGUGGAGCACUA
UCUGAGAGAUCAGCAGCUGCUGGGCAUCUGGGGCUGUAGCGGCAAGCUGAUCUGCUGUACCAAUGUGC
CCUGGAACUCUAGCUGGUCUAAUCGCAACCUGAGCGAGAUCUGGGACAAUAUGACCUGGCUGCAGUGG
GAUAAGGAGAUCUCCAACUACACACAGAUCAUCUAUGGCCUGCUGGAAGAAUCUCAGAAUCAGCAGGAA
AAGAAUGAACAGGAUCUGCUGGCACUGGAUGGAGGCAGCGGCAGCGGCAGCGGGGACCCUGAGUUUAC
CAAAAAUGCUCUGAAUGUCGUCAAAAAUGAUCUGAUUGCUAAGGUGGACCAGCUGAGCGGAGAGCAGG
AGGUGCUGAGGGGCGAGCUGGAGGCCGCCAAGCAGGCAAAGGUGAAACUGGAAAACCGAAUCAAGGAAC
UGGAAGAAGAACUGAAAAGAGUCUGAUAA (SEQ ID NO: 176)
BG505_MD39_IC2 (amino acid, dna, rna)
MDWTWILFLVAAATRVHSAENLWVTVYYGVPVWKDAETTLFCASDAKAYETEKHNVWATHACVPTDPNPQEIH
LENVTEEFNMWKNNMVEQMHEDIISLWDQSLKPCVKLTPLCVTLQCTNVTNNITDDMRGELKNCSFNMTTELR
DKKQKVYSLFYRLDVVQINENQGNRSNNSNKEYRLINCNTSAITQACPKVSFEPIPIHYCAPAGFAILKCKDKKFNGT
GPCPSVSTVQCTHGIKPVVSTQLLLNGSLAEEEVIIRSENITNNAKNILVQLNTPVQINCTRPNNNTVKSIRIGPGQAF
YYTGDIIGDIRQAHCNVSKATWNETLGKVVKQLRKHFGNNTIIRFAQSSGGDLEVTTHSFNCGGEFFYCNTSGLFNS
TWISNTSVQGSNSTGSNDSITLPCRIKQIINMWQRIGQAMYAPPIQGVIRCVSNITGLILTRDGGSTNSTTETFRPG
GGDMRDNWRSELYKYKVVKIEPLGVAPTRCKRRVVGRRRRRRAVGIGAVSLGFLGAAGSTMGAASMTLTVQAR
NLLSGIVQQQSNLLRAPEPQQHLLKDTHWGIKQLQARVLAVEHYLRDQQLLGIWGCSGKLICCTNVPWNSSWSN
RNLSEIWDNMTWLQWDKEISNYTQIIYGLLEESQNQQEKNEQDLLALDGGSGSGSGADPKKVLDKAKDQAENRV
RELKQKLEELYKEARKLDLTQEMRRKLELRYIAAMLMAIGDIYNAIRQAKQEADKLKKAGLVNSQQLDELKRRLEEL
KEEASRKARDYGREFQLKLEYGGGSGSGSGGKIEQILQKIEKILQKIEWILQKIEQILQG** (SEQ ID NO: 180)
atggactggacatggattctgttcctggtcgctgccgctacaagagtgcattccgccgaaaacctgtgggtcaccgtctactatggagtgcccg
tgtggaaggacgccgagactacgctgttctgcgccagcgatgccaaggcctacgagacagagaagcacaacgtgtgggcaacccacgcatgcgt
gcctacagacccaaacccccaggagatccacctggagaatgtgacagaggagtttaacatgtggaagaacaatatggtggagcagatgcacg
aggacatcatctccctgtgggatcagtctctgaagccctgcgtgaagctgacccctctgtgcgtgacactgcagtgtaccaacgtgacaaacaat
atcaccgacgatatgcggggcgagctgaagaattgtagcttcaacatgaccacagagctgagggacaagaagcagaaggtgtactccctgtttt
atagactggatgtggtgcagatcaatgagaaccagggcaatcggtctaacaatagcaacaaggagtaccgcctgatcaattgcaacacctccgcc
atcacacaggcctgtcctaaggtgtctttcgagcctatcccaatccactattgcgccccagccggcttcgccatcctgaagtgtaaggataaga
agtttaacggaaccggaccatgcccttccgtgtctaccgtgcagtgtacacacggcatcaagcctgtggtgtctacacagctgctgctgaatggc
agcctggccgaggaggaagtgatcatcaggtctgagaacatcaccaacaatgccaagaatatcctggtgcagctgaacacaccagtgcagatca
attgcacccggcccaacaataacacagtgaagtctatccgcatcggcccaggccaggccttttactataccggcgacatcatcggcgatatcaga
caggcccactgtaatgtgagcaaggccacctggaacgagacactgggcaaggtggtgaagcagctgaggaagcacttcggcaataacaccat
catcagatttgcacagagctccggcggcgacctggaggtgaccacacactccttcaattgcggcggcgagttcttttactgtaacacaagcggcc
tgtttaattccacctggatctccaacacatctgtgcagggcagcaattccaccggcagcaacgattccatcacactgccatgccggatcaagcag
atcatcaacatgtggcagcgcatcggccaggccatgtatgcccctcccatccagggcgtgatcagatgcgtgagcaatatcaccggcctgatcct
gacacgcgacggcggctctaccaacagcaccacagagacattccggcccggcggcggcgacatgagggataactggagatctgagctgtaca
agtataaggtggtgaagatcgagcctctgggagtggcaccaaccaggtgcaagaggagagtggtgggccggcgcaggagacggcgcgcagtg
ggcatcggagccgtgtccctgggctttctgggagcagcaggctccacaatgggagcagcctctatgaccctgacagtgcaggccaggaatctgc
tgagcggcatcgtgcagcagcagtccaacctgctgagagccccagagccccagcagcacctgctgaaggacacccactggggcatcaagcagc
tgcaggccagggtgctggcagtggagcactatctgagagatcagcagctgctgggcatctggggctgtagcggcaagctgatctgctgtaccaa
tgtgccctggaactctagctggtctaatcgcaacctgagcgagatctgggacaatatgacctggctgcagtgggataaggagatctccaactaca
cacagatcatctatggcctgctggaagaatctcagaatcagcaggaaaagaatgaacaggatctgctggcactggatggcggaagcggaagtg
gaagcggagccgaccccaagaaggtgctggataaagccaaagatcaggcagaaaatagagtcagggaactgaagcagaagctggaggagc
tgtacaaggaggcccggaagctggacctgacccaggagatgaggagaaagctggagctgcgctacatcgccgccatgctgatggccatcggcg
acatctataacgccatcaggcaggccaagcaggaggccgataagctgaagaaggccggcctggtgaatagccagcagctggacgagctgaag
cggcgcctggaggagctgaaggaggaggccagcaggaaggccagagattacggcagggagttccagctgaagctggagtatggcggcggca
gcggctccggctctggcggcaagatcgagcagatcctgcagaagatcgaaaagatcctgcagaagattgagtggattctgcagaagattgaac
agatcctgcaggggtgataa (SEQ ID NO: 178)
AUGGACUGGACAUGGAUUCUGUUCCUGGUCGCUGCCGCUACAAGAGUGCAUUCCGCCGAAAACCUGUG
GGUCACCGUCUACUAUGGAGUGCCCGUGUGGAAGGACGCCGAGACUACGCUGUUCUGCGCCAGCGAUG
CCAAGGCCUACGAGACAGAGAAGCACAACGUGUGGGCAACCCACGCAUGCGUGCCUACAGACCCAAACCC
CCAGGAGAUCCACCUGGAGAAUGUGACAGAGGAGUUUAACAUGUGGAAGAACAAUAUGGUGGAGCAGA
UGCACGAGGACAUCAUCUCCCUGUGGGAUCAGUCUCUGAAGCCCUGCGUGAAGCUGACCCCUCUGUGCG
UGACACUGCAGUGUACCAACGUGACAAACAAUAUCACCGACGAUAUGCGGGGCGAGCUGAAGAAUUGUA
GCUUCAACAUGACCACAGAGCUGAGGGACAAGAAGCAGAAGGUGUACUCCCUGUUUUAUAGACUGGAU
GUGGUGCAGAUCAAUGAGAACCAGGGCAAUCGGUCUAACAAUAGCAACAAGGAGUACCGCCUGAUCAAU
UGCAACACCUCCGCCAUCACACAGGCCUGUCCUAAGGUGUCUUUCGAGCCUAUCCCAAUCCACUAUUGCG
CCCCAGCCGGCUUCGCCAUCCUGAAGUGUAAGGAUAAGAAGUUUAACGGAACCGGACCAUGCCCUUCCG
UGUCUACCGUGCAGUGUACACACGGCAUCAAGCCUGUGGUGUCUACACAGCUGCUGCUGAAUGGCAGCC
UGGCCGAGGAGGAAGUGAUCAUCAGGUCUGAGAACAUCACCAACAAUGCCAAGAAUAUCCUGGUGCAGC
UGAACACACCAGUGCAGAUCAAUUGCACCCGGCCCAACAAUAACACAGUGAAGUCUAUCCGCAUCGGCCC
AGGCCAGGCCUUUUACUAUACCGGCGACAUCAUCGGCGAUAUCAGACAGGCCCACUGUAAUGUGAGCAA
GGCCACCUGGAACGAGACACUGGGCAAGGUGGUGAAGCAGCUGAGGAAGCACUUCGGCAAUAACACCAU
CAUCAGAUUUGCACAGAGCUCCGGCGGCGACCUGGAGGUGACCACACACUCCUUCAAUUGCGGCGGCGA
GUUCUUUUACUGUAACACAAGCGGCCUGUUUAAUUCCACCUGGAUCUCCAACACAUCUGUGCAGGGCA
GCAAUUCCACCGGCAGCAACGAUUCCAUCACACUGCCAUGCCGGAUCAAGCAGAUCAUCAACAUGUGGCA
GCGCAUCGGCCAGGCCAUGUAUGCCCCUCCCAUCCAGGGCGUGAUCAGAUGCGUGAGCAAUAUCACCGG
CCUGAUCCUGACACGCGACGGCGGCUCUACCAACAGCACCACAGAGACAUUCCGGCCCGGCGGCGGCGAC
AUGAGGGAUAACUGGAGAUCUGAGCUGUACAAGUAUAAGGUGGUGAAGAUCGAGCCUCUGGGAGUGG
CACCAACCAGGUGCAAGAGGAGAGUGGUGGGCCGGCGCAGGAGACGGCGCGCAGUGGGCAUCGGAGCC
GUGUCCCUGGGCUUUCUGGGAGCAGCAGGCUCCACAAUGGGAGCAGCCUCUAUGACCCUGACAGUGCA
GGCCAGGAAUCUGCUGAGCGGCAUCGUGCAGCAGCAGUCCAACCUGCUGAGAGCCCCAGAGCCCCAGCA
GCACCUGCUGAAGGACACCCACUGGGGCAUCAAGCAGCUGCAGGCCAGGGUGCUGGCAGUGGAGCACUA
UCUGAGAGAUCAGCAGCUGCUGGGCAUCUGGGGCUGUAGCGGCAAGCUGAUCUGCUGUACCAAUGUGC
CCUGGAACUCUAGCUGGUCUAAUCGCAACCUGAGCGAGAUCUGGGACAAUAUGACCUGGCUGCAGUGG
GAUAAGGAGAUCUCCAACUACACACAGAUCAUCUAUGGCCUGCUGGAAGAAUCUCAGAAUCAGCAGGAA
AAGAAUGAACAGGAUCUGCUGGCACUGGAUGGCGGAAGCGGAAGUGGAAGCGGAGCCGACCCCAAGAA
GGUGCUGGAUAAAGCCAAAGAUCAGGCAGAAAAUAGAGUCAGGGAACUGAAGCAGAAGCUGGAGGAGC
UGUACAAGGAGGCCCGGAAGCUGGACCUGACCCAGGAGAUGAGGAGAAAGCUGGAGCUGCGCUACAUC
GCCGCCAUGCUGAUGGCCAUCGGCGACAUCUAUAACGCCAUCAGGCAGGCCAAGCAGGAGGCCGAUAAG
CUGAAGAAGGCCGGCCUGGUGAAUAGCCAGCAGCUGGACGAGCUGAAGCGGCGCCUGGAGGAGCUGAA
GGAGGAGGCCAGCAGGAAGGCCAGAGAUUACGGCAGGGAGUUCCAGCUGAAGCUGGAGUAUGGCGGCG
GCAGCGGCUCCGGCUCUGGCGGCAAGAUCGAGCAGAUCCUGCAGAAGAUCGAAAAGAUCCUGCAGAAGA
UUGAGUGGAUUCUGCAGAAGAUUGAACAGAUCCUGCAGGGGUGAUAA (SEQ ID NO: 179)
Global panel of trimers
Parts of sequences
Leader sequences
IgE
MDWTWILFLVAAATRVHS (SEQ ID NO: 7)
Linkers
Link 14 (same as MD3)
GSHSGSGGSGSGGHA (SEQ ID NO: 13)
There are no repeats in the gp120 or gp41 ecto between these sequences.
I have highlighted them the same as the BG505_MD39. All of these are soluble
IgE-gp120-linker-gp41 ecto (bold are glycan mutations)
Full length sequences
TRO11_AY835445_MD39_L14G8 (amino acid, dna, rna)
MDWTWILFLVAAATRVHSQGQLWVTVYYGVPVWKDASTTLFCASDAKAYDTEVHNVWATHACVPTDP
NPQEVVLGNVTENFNMWKNNMVDQMHEDIISLWDQSLKPCVKLTPLCVTLNCTDNITNTNTNSSKNSS
THSYNNSLEGEMKNCSFNITAGIRDKVKKEYALFYKLDVVPIEEDKDTNKTTYRLRSCNTSVITQACPKVTFE
PIPIHYCAPAGFAILKCNDKKFNGTGPCTNVSTVQCTHGIRPVVSTQLLLNGSLAEEEVIIRSENFTNNAKTII
VQLNESIAINCTRPNNNTVRSIHIGPGRAFYYTGDIIGDIRQAHCNISRTEWNSTLRQIVTKLREQLGDPNKT
IIFAQSSGGDTEITMHSFNCGGEFFYCNTTKLFNSTWNGNNTTESDSTGENITLPCRIKQIINLWQEVGKA
MYAPPIKGQISCSSNITGLLLTRDGGNNNSSGPETFRPGGGNMKDNWRSELYKYKVIKIEPLGVAPTRCKR
RVVGSHSGSGGSGSGGHAAVGTLGAMSLGFLGAAGSTMGAASVTLTVQARLLLSGIVQQQNNLLRAPE
PQQHMLQDTHWGIKQLQARVLAVEHYLRDQQLLGIWGCSGKLICCTNVPWNASWSNKSLNNIWENM
TWMNWSREIDNYTDLIYILLEKSQIQQEKNNQSLLELD** (SEQ ID NO: 183)
atggattggacttggattctgtttctggtcgctgctgctactcgggtgcattctcagggccagctgtgggtcactgtctactacggcgtg
ccagtgtggaaggacgcctctaccacactgttttgcgccagcgacgccaaggcctacgatacagaggtgcacaacgtgtgggcaac
acacgcatgcgtgccaaccgatccaaatccccaggaggtggtgctgggcaacgtgaccgagaacttcaatatgtggaagaacaata
tggtggaccagatgcacgaggatatcatctctctgtgggaccagagcctgaagccctgcgtgaagctgacccctctgtgcgtgacact
gaattgtaccgataacatcaccaacacaaataccaacagctccaagaactctagcacacactcctataacaattctctggagggcga
gatgaagaattgttcctttaacatcaccgccggcatccgggacaaggtgaagaaggagtacgccctgttctataagctggatgtggtg
cccatcgaggaggacaaggatacaaataagaccacataccggctgcgcagctgcaacacatccgtgatcacccaggcctgtcctaa
ggtgacctttgagcctatcccaatccactattgcgccccagccggcttcgccatcctgaagtgtaatgacaagaagtttaacggcaca
ggcccctgcaccaacgtgtctacagtgcagtgtacccacggcatcaggcctgtggtgtccacccagctgctgctgaatggctctctgg
ccgaggaggaagtgatcatcagaagcgagaactttacaaacaatgccaagaccatcatcgtgcagctgaatgagtctatcgccatc
aactgcacaaggccaaacaataacaccgtgagaagcatccacatcggaccaggaagggccttctactataccggcgacatcatcgg
cgatatcaggcaggcccactgtaatatctccagaacagagtggaactctaccctgcggcagatcgtgacaaagctgcgcgagcagc
tgggcgaccctaacaagaccatcatcttcgcccagtcctctggcggcgatacagagatcaccatgcactcctttaattgcggcggcga
gttcttttactgtaacaccacaaagctgttcaattctacctggaacggcaataacaccacagagtccgactctacaggcgagaatatc
accctgccatgccggatcaagcagatcatcaacctgtggcaggaagtgggcaaggccatgtatgcccctcccatcaagggccagat
ctcctgtagctccaacatcacaggcctgctgctgacccgcgacggcggaaataacaattctagcggaccagagacattcaggcctgg
cggcggcaatatgaaggataactggagaagcgagctgtacaagtataaagtgatcaagatcgagcctctgggagtggcaccaacc
aggtgcaagaggagagtggtgggcagccactccggctctggcggcagcggctccggcggccacgcagcagtgggcacactgggcg
ccatgagcctgggcttcctgggagcagcaggcagcaccatgggagcagcatccgtgacactgaccgtgcaggcaaggctgctgctg
tccggcatcgtgcagcagcagaacaatctgctgagggcaccagagcctcagcagcacatgctgcaggacacacactggggcatca
agcagctgcaggcccgggtgctggcagtggagcactacctgcgcgatcagcagctgctgggcatctggggctgtagcggcaagctg
atctgctgtaccaatgtgccttggaacgcctcttggagcaataagagcctgaacaatatctgggagaatatgacatggatgaactggt
ccagagagatcgacaactacaccgatctgatctatatcctgctggagaagtcacagattcagcaggagaagaacaatcagagcctg
ctggaactggattgataa (SEQ ID NO: 181)
AUGGAUUGGACUUGGAUUCUGUUUCUGGUCGCUGCUGCUACUCGGGUGCAUUCUCAGGGCCA
GCUGUGGGUCACUGUCUACUACGGCGUGCCAGUGUGGAAGGACGCCUCUACCACACUGUUUUG
CGCCAGCGACGCCAAGGCCUACGAUACAGAGGUGCACAACGUGUGGGCAACACACGCAUGCGUG
CCAACCGAUCCAAAUCCCCAGGAGGUGGUGCUGGGCAACGUGACCGAGAACUUCAAUAUGUGGA
AGAACAAUAUGGUGGACCAGAUGCACGAGGAUAUCAUCUCUCUGUGGGACCAGAGCCUGAAGC
CCUGCGUGAAGCUGACCCCUCUGUGCGUGACACUGAAUUGUACCGAUAACAUCACCAACACAAA
UACCAACAGCUCCAAGAACUCUAGCACACACUCCUAUAACAAUUCUCUGGAGGGCGAGAUGAAG
AAUUGUUCCUUUAACAUCACCGCCGGCAUCCGGGACAAGGUGAAGAAGGAGUACGCCCUGUUC
UAUAAGCUGGAUGUGGUGCCCAUCGAGGAGGACAAGGAUACAAAUAAGACCACAUACCGGCUGC
GCAGCUGCAACACAUCCGUGAUCACCCAGGCCUGUCCUAAGGUGACCUUUGAGCCUAUCCCAAU
CCACUAUUGCGCCCCAGCCGGCUUCGCCAUCCUGAAGUGUAAUGACAAGAAGUUUAACGGCACA
GGCCCCUGCACCAACGUGUCUACAGUGCAGUGUACCCACGGCAUCAGGCCUGUGGUGUCCACCC
AGCUGCUGCUGAAUGGCUCUCUGGCCGAGGAGGAAGUGAUCAUCAGAAGCGAGAACUUUACAA
ACAAUGCCAAGACCAUCAUCGUGCAGCUGAAUGAGUCUAUCGCCAUCAACUGCACAAGGCCAAA
CAAUAACACCGUGAGAAGCAUCCACAUCGGACCAGGAAGGGCCUUCUACUAUACCGGCGACAUC
AUCGGCGAUAUCAGGCAGGCCCACUGUAAUAUCUCCAGAACAGAGUGGAACUCUACCCUGCGGC
AGAUCGUGACAAAGCUGCGCGAGCAGCUGGGCGACCCUAACAAGACCAUCAUCUUCGCCCAGUC
CUCUGGCGGCGAUACAGAGAUCACCAUGCACUCCUUUAAUUGCGGCGGCGAGUUCUUUUACUG
UAACACCACAAAGCUGUUCAAUUCUACCUGGAACGGCAAUAACACCACAGAGUCCGACUCUACAG
GCGAGAAUAUCACCCUGCCAUGCCGGAUCAAGCAGAUCAUCAACCUGUGGCAGGAAGUGGGCAA
GGCCAUGUAUGCCCCUCCCAUCAAGGGCCAGAUCUCCUGUAGCUCCAACAUCACAGGCCUGCUG
CUGACCCGCGACGGCGGAAAUAACAAUUCUAGCGGACCAGAGACAUUCAGGCCUGGCGGCGGCA
AUAUGAAGGAUAACUGGAGAAGCGAGCUGUACAAGUAUAAAGUGAUCAAGAUCGAGCCUCUGG
GAGUGGCACCAACCAGGUGCAAGAGGAGAGUGGUGGGCAGCCACUCCGGCUCUGGCGGCAGCG
GCUCCGGCGGCCACGCAGCAGUGGGCACACUGGGCGCCAUGAGCCUGGGCUUCCUGGGAGCAGC
AGGCAGCACCAUGGGAGCAGCAUCCGUGACACUGACCGUGCAGGCAAGGCUGCUGCUGUCCGGC
AUCGUGCAGCAGCAGAACAAUCUGCUGAGGGCACCAGAGCCUCAGCAGCACAUGCUGCAGGACA
CACACUGGGGCAUCAAGCAGCUGCAGGCCCGGGUGCUGGCAGUGGAGCACUACCUGCGCGAUCA
GCAGCUGCUGGGCAUCUGGGGCUGUAGCGGCAAGCUGAUCUGCUGUACCAAUGUGCCUUGGAA
CGCCUCUUGGAGCAAUAAGAGCCUGAACAAUAUCUGGGAGAAUAUGACAUGGAUGAACUGGUC
CAGAGAGAUCGACAACUACACCGAUCUGAUCUAUAUCCUGCUGGAGAAGUCACAGAUUCAGCAG
GAGAAGAACAAUCAGAGCCUGCUGGAACUGGAUUGAUAA (SEQ ID NO: 182)
X2278_FJ817366_MD39_L14G8 (amino acid, dna, rna)
MDWTWILFLVAAATRVHSTNNLWVTVYYGVPVWKEATTTLFCASEAKAYDTEVHNIWATHACVPTDPN
PQEMELKNVTENFNMWKNNMVEQMHEDIISLWDQSLKPCVKLTPLCVTLDCTNINSTNSTNNTSSNSK
MEETIGVIKNCSFNVTTNIRDKVKKENALFYSLDLVSIGNSNTSYRLISCNTSIITQACPKVSFDPIPIHYCAPA
GFAILKCRDKKFNGTGPCRNVSSVQCTHGIRPVVSTQLLLNGSLAEEEIIIRSANLTDNAKTIIIQLNETIQINC
TRPNNNTVRSIPIGPGRTFYYTGDIIGDIRKAYCNISATKWNNTLRQIAEKLREKFNKTIIFAQSSGGDPEVVR
HTFNCGGEFFYCNSSQLFNSTWYSNGTSNGGLNNSANITLPCRIKQIINLWQEVGKAMYAPPIKGVINCLS
NITGIILTRDGGENNGTTETFRPGGGDMRDNWRSELYKYKVVKIEPLGIAPTKCKRRVVGSHSGSGGSGSG
GHAAVGLGAVSLGFLGLAGSTMGAASVTLTVQARLLLSGIVQQQNNLLRAPEPQQQLLQDTHWGIKQL
QARVLALEHYLKDQQLLGIWGCSGKLICCTTVPWNASWSNKSYNQIWNNMTWMNWSREIDNYTNLIY
NLIEESQSQQEKNNLSLLQLD** (SEQ ID NO: 186)
atggactggacctggattctgttcctggtcgccgctgctacaagagtgcattctacaaataacctgtgggtgactgtctactatggagt
gcccgtgtggaaggaggccaccacaaccctgttctgcgccagcgaggccaaggcctacgacacagaggtgcacaacatctgggcc
acccacgcctgcgtgcctacagatccaaacccccaggagatggagctgaagaatgtgaccgagaacttcaacatgtggaagaaca
atatggtggagcagatgcacgaggacatcatcagcctgtgggatcagtccctgaagccctgcgtgaagctgacacctctgtgcgtga
ccctggattgtacaaatatcaacagcacaaactccaccaacaatacaagctccaattctaagatggaggagacaatcggcgtgatca
agaattgtagcttcaacgtgacaaccaatatccgggacaaggtgaagaaggagaacgccctgttttactctctggatctggtgagcat
cggcaattctaacaccagctatcgcctgatctcctgcaatacctctatcatcacacaggcctgtccaaaggtgagcttcgaccctatcc
caatccactactgcgcaccagcaggattcgcaatcctgaagtgtagggataagaagtttaacggcaccggcccttgcagaaacgtga
gcagcgtgcagtgtacacacggcatcaggccagtggtgagcacccagctgctgctgaacggctccctggcagaggaggagatcatc
atcagatccgccaacctgaccgacaatgccaagacaatcatcatccagctgaacgagacaatccagatcaattgcacaaggcccaa
caataacaccgtgagaagcatcccaatcggccccggccggaccttttactatacaggcgacatcatcggcgatatccgcaaggccta
ctgtaacatctccgccaccaagtggaataacacactgcggcagatcgccgagaagctgcgcgagaagttcaacaagacaatcatct
ttgcccagtcctctggcggcgatccagaggtggtgaggcacaccttcaattgcggcggcgagttcttttactgtaacagctcccagctg
tttaatagcacatggtattccaacggcacctctaatggcggcctgaataacagcgccaacatcaccctgccctgcagaatcaagcag
atcatcaatctgtggcaggaagtgggcaaggccatgtatgcccctcccatcaagggcgtgatcaactgtctgtccaatatcaccggca
tcatcctgacaagggacggcggcgagaataacggcacaaccgagacattcagacccggcggcggcgacatgagggataactggc
gctctgagctgtacaagtataaggtggtgaagatcgagcctctgggcatcgccccaaccaagtgcaagaggagagtggtgggctctc
acagcggctccggcggctctggcagcggcggccacgcagcagtgggcctgggagccgtgtctctgggctttctgggcctggcaggct
ccacaatgggagcagcctctgtgacactgaccgtgcaggcaaggctgctgctgagcggcatcgtgcagcagcagaataacctgctg
agggcaccagagcctcagcagcagctgctgcaggacacccactggggcatcaagcagctgcaggcccgggtgctggccctggagc
actacctgaaggatcagcagctgctgggcatctggggctgttccggcaagctgatctgctgtacaaccgtgccatggaacgcctcctg
gtctaacaagtcctataatcagatctggaataacatgacatggatgaactggagcagggagatcgacaattacaccaacctgatcta
taatctgattgaagagtcacagtcacagcaggaaaagaacaacctgagcctgctgcagctggactgataa (SEQ ID NO: 188)
AUGGACUGGACCUGGAUUCUGUUCCUGGUCGCCGCUGCUACAAGAGUGCAUUCUACAAAUAAC
CUGUGGGUGACUGUCUACUAUGGAGUGCCCGUGUGGAAGGAGGCCACCACAACCCUGUUCUGC
GCCAGCGAGGCCAAGGCCUACGACACAGAGGUGCACAACAUCUGGGCCACCCACGCCUGCGUGCC
UACAGAUCCAAACCCCCAGGAGAUGGAGCUGAAGAAUGUGACCGAGAACUUCAACAUGUGGAAG
AACAAUAUGGUGGAGCAGAUGCACGAGGACAUCAUCAGCCUGUGGGAUCAGUCCCUGAAGCCC
UGCGUGAAGCUGACACCUCUGUGCGUGACCCUGGAUUGUACAAAUAUCAACAGCACAAACUCCA
CCAACAAUACAAGCUCCAAUUCUAAGAUGGAGGAGACAAUCGGCGUGAUCAAGAAUUGUAGCUU
CAACGUGACAACCAAUAUCCGGGACAAGGUGAAGAAGGAGAACGCCCUGUUUUACUCUCUGGAU
CUGGUGAGCAUCGGCAAUUCUAACACCAGCUAUCGCCUGAUCUCCUGCAAUACCUCUAUCAUCA
CACAGGCCUGUCCAAAGGUGAGCUUCGACCCUAUCCCAAUCCACUACUGCGCACCAGCAGGAUU
CGCAAUCCUGAAGUGUAGGGAUAAGAAGUUUAACGGCACCGGCCCUUGCAGAAACGUGAGCAG
CGUGCAGUGUACACACGGCAUCAGGCCAGUGGUGAGCACCCAGCUGCUGCUGAACGGCUCCCUG
GCAGAGGAGGAGAUCAUCAUCAGAUCCGCCAACCUGACCGACAAUGCCAAGACAAUCAUCAUCCA
GCUGAACGAGACAAUCCAGAUCAAUUGCACAAGGCCCAACAAUAACACCGUGAGAAGCAUCCCAA
UCGGCCCCGGCCGGACCUUUUACUAUACAGGCGACAUCAUCGGCGAUAUCCGCAAGGCCUACUG
UAACAUCUCCGCCACCAAGUGGAAUAACACACUGCGGCAGAUCGCCGAGAAGCUGCGCGAGAAG
UUCAACAAGACAAUCAUCUUUGCCCAGUCCUCUGGCGGCGAUCCAGAGGUGGUGAGGCACACCU
UCAAUUGCGGCGGCGAGUUCUUUUACUGUAACAGCUCCCAGCUGUUUAAUAGCACAUGGUAUU
CCAACGGCACCUCUAAUGGCGGCCUGAAUAACAGCGCCAACAUCACCCUGCCCUGCAGAAUCAAG
CAGAUCAUCAAUCUGUGGCAGGAAGUGGGCAAGGCCAUGUAUGCCCCUCCCAUCAAGGGCGUG
AUCAACUGUCUGUCCAAUAUCACCGGCAUCAUCCUGACAAGGGACGGCGGCGAGAAUAACGGCA
CAACCGAGACAUUCAGACCCGGCGGCGGCGACAUGAGGGAUAACUGGCGCUCUGAGCUGUACAA
GUAUAAGGUGGUGAAGAUCGAGCCUCUGGGCAUCGCCCCAACCAAGUGCAAGAGGAGAGUGGU
GGGCUCUCACAGCGGCUCCGGCGGCUCUGGCAGCGGCGGCCACGCAGCAGUGGGCCUGGGAGC
CGUGUCUCUGGGCUUUCUGGGCCUGGCAGGCUCCACAAUGGGAGCAGCCUCUGUGACACUGAC
CGUGCAGGCAAGGCUGCUGCUGAGCGGCAUCGUGCAGCAGCAGAAUAACCUGCUGAGGGCACCA
GAGCCUCAGCAGCAGCUGCUGCAGGACACCCACUGGGGCAUCAAGCAGCUGCAGGCCCGGGUGC
UGGCCCUGGAGCACUACCUGAAGGAUCAGCAGCUGCUGGGCAUCUGGGGCUGUUCCGGCAAGC
UGAUCUGCUGUACAACCGUGCCAUGGAACGCCUCCUGGUCUAACAAGUCCUAUAAUCAGAUCUG
GAAUAACAUGACAUGGAUGAACUGGAGCAGGGAGAUCGACAAUUACACCAACCUGAUCUAUAAU
CUGAUUGAAGAGUCACAGUCACAGCAGGAAAAGAACAACCUGAGCCUGCUGCAGCUGGACUGAU
AA (SEQ ID NO: 189)
398F1_HM215312_MD39_L14G8 (amino acid, dna, rna)
MDWTWILFLVAAATRVHSMGNLWVTVYYGVPVWKDAETTLFCASDAKAYHTEVHNVWATHACVPTD
PNPQEINLENVTEEFNMWKNKMVEQMHEDIISLWDQSLKPCVQLTPLCVTLDCQYNVTNINSTSDMAR
EINNCSYNITTELRDREQKVYSLFYRSDIVQMNSDNSSKYRLINCNTSAIKQACPKVTFEPIPIHYCAPAGFAIL
KCKDKEFNGTGPCKNVSTVQCTHGIKPVVSTQLLLNGSLAEEKVIIRSENITDNAKNIIVQLKEPVKINCTRP
NNNTVKSVRIGPGQTFYYTGEIIGDIRQAHCNVSKAHWENTLQEVANQLKLMIHSNKTIIFANSSGGDLEIT
THSFNCGGEFFYCYTSGLFNYTFNDTSTNSTESKSNDTITLQCRIKQIINMWQRAGQAVYAPPIPGIIRCESN
ITGLILTRDGGNNNSNTNETFRPGGGDMRDNWRSELYRYKVVKIEPIGVAPTTCKRRVVGSHSGSGGSGS
GGHAAVGIGAVSLGFLGAAGSTMGAASITLTVQARQLLSGIVQQQSNLLRAPEPQQHLLKDTHWGIKQL
KARVLAVEHYLKDQQLLGIWGCSGKLICCTNVPWNSSWSNKSLGEIWDNMTWLNWSKEIENYTQIIYELI
EESQNQQEKNNQSLLALD** (SEQ ID NO: 189)
atggactggacttggattctgtttctggtcgcagccgcaactagagtgcatagcatgggcaacctgtgggtcaccgtgtattacggggt
gccagtgtggaaggacgccgagactacgctgttctgcgcctccgatgccaaggcctaccacacagaggtgcacaacgtgtgggcaa
cccacgcatgcgtgccaacagacccaaatccccaggagatcaacctggagaatgtgaccgaggagtttaacatgtggaagaataag
atggtggagcagatgcacgaggacatcatctccctgtgggatcagtctctgaagccttgcgtgcagctgaccccactgtgcgtgacac
tggactgtcagtacaacgtgaccaacatcaatagcacatccgatatggccagggagatcaacaattgtagctataatatcaccacag
agctgcgggatcgcgagcagaaagtgtacagcctgttctataggtccgacatcgtgcagatgaactccgataatagctccaagtaca
gactgatcaactgcaatacctctgccatcaagcaggcctgtccaaaggtgacatttgagcctatcccaatccactattgcgcaccagc
aggattcgcaatcctgaagtgtaaggacaaggagtttaacggcaccggcccttgcaagaacgtgagcaccgtgcagtgtacacacg
gcatcaagccagtggtgagcacacagctgctgctgaacggctccctggccgaggagaaagtgatcatccggtctgagaatatcacc
gataacgccaagaatatcatcgtgcagctgaaggagcccgtgaagatcaactgcacccggcctaacaataacacagtgaagtccgt
gcgcatcggccctggccagaccttctactatacaggcgagatcatcggcgacatccgccaggcccactgtaacgtgtctaaggccca
ctgggagaacaccctgcaggaggtggccaatcagctgaagctgatgatccacagcaacaagacaatcatcttcgccaattctagcg
gcggcgatctggagatcaccacacactcttttaactgcggcggcgagttcttttactgttataccagcggcctgttcaactacaccttca
acgacaccagcacaaactccaccgagtctaagagcaatgataccatcacactgcagtgcaggatcaagcagatcatcaacatgtgg
cagagagcaggacaggccgtgtatgcccctcccatccccggcatcatccggtgtgagagcaatatcaccggcctgatcctgacacgc
gacggcggaaataacaattccaacaccaatgagacattcaggcccggcggcggcgacatgagggataactggagatctgagctgt
acagatataaggtggtgaagatcgagccaatcggcgtggcccccaccacatgcaagaggagagtggtgggctcccactctggcagc
ggcggctccggctctggcggccacgcagccgtgggcatcggagccgtgagcctgggctttctgggagcagcaggctctaccatggg
agcagccagcatcaccctgacagtgcaggcaaggcagctgctgtccggaatcgtgcagcagcagtctaacctgctgagggcaccag
agcctcagcagcacctgctgaaggacacccactggggcatcaagcagctgaaggccagggtgctggccgtggagcactacctgaa
ggatcagcagctgctgggcatctggggctgtagcggcaagctgatctgctgtaccaacgtgccctggaattcctcttggtctaacaag
agcctgggcgagatctgggacaacatgacctggctgaattggtccaaggagatcgagaattacacacagatcatctatgagctgatt
gaagagtcacagaaccagcaggagaaaaacaaccagagcctgctggcactggattgataa (SEQ ID NO: 187)
AUGGACUGGACUUGGAUUCUGUUUCUGGUCGCAGCCGCAACUAGAGUGCAUAGCAUGGGCAAC
CUGUGGGUCACCGUGUAUUACGGGGUGCCAGUGUGGAAGGACGCCGAGACUACGCUGUUCUG
CGCCUCCGAUGCCAAGGCCUACCACACAGAGGUGCACAACGUGUGGGCAACCCACGCAUGCGUG
CCAACAGACCCAAAUCCCCAGGAGAUCAACCUGGAGAAUGUGACCGAGGAGUUUAACAUGUGGA
AGAAUAAGAUGGUGGAGCAGAUGCACGAGGACAUCAUCUCCCUGUGGGAUCAGUCUCUGAAGC
CUUGCGUGCAGCUGACCCCACUGUGCGUGACACUGGACUGUCAGUACAACGUGACCAACAUCAA
UAGCACAUCCGAUAUGGCCAGGGAGAUCAACAAUUGUAGCUAUAAUAUCACCACAGAGCUGCGG
GAUCGCGAGCAGAAAGUGUACAGCCUGUUCUAUAGGUCCGACAUCGUGCAGAUGAACUCCGAU
AAUAGCUCCAAGUACAGACUGAUCAACUGCAAUACCUCUGCCAUCAAGCAGGCCUGUCCAAAGG
UGACAUUUGAGCCUAUCCCAAUCCACUAUUGCGCACCAGCAGGAUUCGCAAUCCUGAAGUGUAA
GGACAAGGAGUUUAACGGCACCGGCCCUUGCAAGAACGUGAGCACCGUGCAGUGUACACACGGC
AUCAAGCCAGUGGUGAGCACACAGCUGCUGCUGAACGGCUCCCUGGCCGAGGAGAAAGUGAUCA
UCCGGUCUGAGAAUAUCACCGAUAACGCCAAGAAUAUCAUCGUGCAGCUGAAGGAGCCCGUGAA
GAUCAACUGCACCCGGCCUAACAAUAACACAGUGAAGUCCGUGCGCAUCGGCCCUGGCCAGACC
UUCUACUAUACAGGCGAGAUCAUCGGCGACAUCCGCCAGGCCCACUGUAACGUGUCUAAGGCCC
ACUGGGAGAACACCCUGCAGGAGGUGGCCAAUCAGCUGAAGCUGAUGAUCCACAGCAACAAGAC
AAUCAUCUUCGCCAAUUCUAGCGGCGGCGAUCUGGAGAUCACCACACACUCUUUUAACUGCGGC
GGCGAGUUCUUUUACUGUUAUACCAGCGGCCUGUUCAACUACACCUUCAACGACACCAGCACAA
ACUCCACCGAGUCUAAGAGCAAUGAUACCAUCACACUGCAGUGCAGGAUCAAGCAGAUCAUCAA
CAUGUGGCAGAGAGCAGGACAGGCCGUGUAUGCCCCUCCCAUCCCCGGCAUCAUCCGGUGUGAG
AGCAAUAUCACCGGCCUGAUCCUGACACGCGACGGCGGAAAUAACAAUUCCAACACCAAUGAGAC
AUUCAGGCCCGGCGGCGGCGACAUGAGGGAUAACUGGAGAUCUGAGCUGUACAGAUAUAAGGU
GGUGAAGAUCGAGCCAAUCGGCGUGGCCCCCACCACAUGCAAGAGGAGAGUGGUGGGCUCCCAC
UCUGGCAGCGGCGGCUCCGGCUCUGGCGGCCACGCAGCCGUGGGCAUCGGAGCCGUGAGCCUG
GGCUUUCUGGGAGCAGCAGGCUCUACCAUGGGAGCAGCCAGCAUCACCCUGACAGUGCAGGCAA
GGCAGCUGCUGUCCGGAAUCGUGCAGCAGCAGUCUAACCUGCUGAGGGCACCAGAGCCUCAGCA
GCACCUGCUGAAGGACACCCACUGGGGCAUCAAGCAGCUGAAGGCCAGGGUGCUGGCCGUGGA
GCACUACCUGAAGGAUCAGCAGCUGCUGGGCAUCUGGGGCUGUAGCGGCAAGCUGAUCUGCUG
UACCAACGUGCCCUGGAAUUCCUCUUGGUCUAACAAGAGCCUGGGCGAGAUCUGGGACAACAU
GACCUGGCUGAAUUGGUCCAAGGAGAUCGAGAAUUACACACAGAUCAUCUAUGAGCUGAUUGA
AGAGUCACAGAACCAGCAGGAGAAAAACAACCAGAGCCUGCUGGCACUGGAUUGAUAA (SEQ ID
NO: 188)
246F3_HM215279_MD39_L14G8 (amino acid, dna, rna)
MDWTWILFLVAAATRVHSMQDLWVTVYYGVPVWKDAKTTLFCASDAKAYEKEVHNVWATHACVPTD
PNPQEIVMANVTEEFNMWKNNMVEQMHEDIISLWDQSLKPCVKLTPLCVTLDCKDYNYSITNNSTGME
GEIKNCSYNITTELRDKRQKVYSLFYRLDVVQINDSNDRNNSQYRLINCNTTTMTQACPKVTFDPIPIHYCA
PAGFAILKCNNKTFNGKGPCNNVSSVQCTHGIKPVVSTQLLLNGSLAEKEIIIRSENLTDNVKTIIVHLNESVE
INCTRPNNNTVKSVRIGPGQTFYYTGDIIGNIRQAHCTVNKTEWNTALTRVSKKLKEYFPNKTIAFQPSSGG
DLEITTFSFNCRGEFFYCNTSDLFNGTFNETSGQFNSTFNSTLQCRIKQIINMWQEVGQAMYAPPIAGSITC
ISNITGLILTRDGGNTNSTKETFRPGGGNMRDNWRSELYKYKVVKIEPLGVAPTKCRRRVVGSHSGSGGSG
SGGHAAVGIGAVSIGFLGAAGSTMGAASITLTVQARQLLSGIVQQQSNLLRAPEPQQHLLKDTHWGIKQL
QARVLAVEHYLKDQQLLGIWGCSGKLICCTNVPWNSSWSNKSQDEIWDNMTWLNWSKEISNYTQIIYNL
IEESQTQQELNNRSLLALD** (SEQ ID NO: 192)
atggactggacttggattctgtttctggtcgcagccgctactcgggtgcactctatgcaggacctgtgggtgaccgtctattatggggtg
ccagtgtggaaggacgccaagaccacactgttctgcgcctccgatgccaaggcctacgagaaggaggtgcacaacgtgtgggcaac
ccacgcatgcgtgccaacagacccaaacccccaggagatcgtgatggccaatgtgaccgaggagtttaacatgtggaagaacaata
tggtggagcagatgcacgaggacatcatctctctgtgggatcagagcctgaagccttgcgtgaagctgaccccactgtgcgtgacac
tggactgtaaggattacaactattccatcaccaacaattctacaggcatggagggcgagatcaagaattgttcttataacatcaccac
agagctgcgcgacaagaggcagaaagtgtacagcctgttctatcgcctggatgtggtgcagatcaatgactctaacgatcgcaacaa
tagccagtacaggctgatcaattgcaacaccacaaccatgacccaggcctgtcctaaggtgacatttgaccctatcccaatccactat
tgcgccccagccggcttcgccatcctgaagtgtaacaataagacctttaatggcaagggcccctgcaacaatgtgagctccgtgcagt
gtacccacggcatcaagcctgtggtgtctacacagctgctgctgaacggcagcctggccgagaaggagatcatcatcaggagcgag
aatctgaccgacaacgtgaagacaatcatcgtgcacctgaatgagagcgtggagatcaactgcaccagaccaaacaataacacagt
gaagtccgtgcggatcggaccaggacagaccttctactatacaggcgatatcatcggcaatatccgccaggcccactgtaccgtgaa
taagacagagtggaacacagccctgaccagggtgagcaagaagctgaaggagtacttccccaacaagaccatcgcctttcagcctt
ctagcggcggcgacctggagatcacaaccttctcctttaattgcagaggcgagttcttttattgtaacacatccgatctgttcaatggca
cctttaacgagacatctggccagttcaattccacctttaactctacactgcagtgccggatcaagcagatcatcaatatgtggcagga
agtgggacaggcaatgtacgcccctcccatcgcaggcagcatcacctgtatctccaacatcaccggcctgatcctgacacgcgacgg
cggaaatacaaactccaccaaggagacattcaggcctggcggcggcaatatgagagataactggcggtctgagctgtacaagtata
aggtggtgaagatcgagccactgggagtggcaccaaccaagtgcaggagacgggtggtgggcagccactccggctctggcggcag
cggctccggcggccacgcagcagtgggcatcggcgccgtgtctatcggctttctgggagcagcaggctccaccatgggagcagcctc
tatcacactgaccgtgcaggccagacagctgctgagcggcatcgtgcagcagcagtccaacctgctgagggcaccagagcctcagc
agcacctgctgaaggacacccactggggcatcaagcagctgcaggccagggtgctggcagtggagcactacctgaaggatcagca
gctgctgggcatctggggctgtagcggcaagctgatctgctgtacaaatgtgccctggaactcctcttggtctaacaagagccaggac
gagatctgggataatatgacctggctgaactggagcaaggagatctccaattacacacagatcatctataacctgattgaagaatca
cagactcagcaggaactgaataataggtcactgctggcactggattgataa (SEQ ID NO: 190)
AUGGACUGGACUUGGAUUCUGUUUCUGGUCGCAGCCGCUACUCGGGUGCACUCUAUGCAGGAC
CUGUGGGUGACCGUCUAUUAUGGGGUGCCAGUGUGGAAGGACGCCAAGACCACACUGUUCUGC
GCCUCCGAUGCCAAGGCCUACGAGAAGGAGGUGCACAACGUGUGGGCAACCCACGCAUGCGUGC
CAACAGACCCAAACCCCCAGGAGAUCGUGAUGGCCAAUGUGACCGAGGAGUUUAACAUGUGGAA
GAACAAUAUGGUGGAGCAGAUGCACGAGGACAUCAUCUCUCUGUGGGAUCAGAGCCUGAAGCC
UUGCGUGAAGCUGACCCCACUGUGCGUGACACUGGACUGUAAGGAUUACAACUAUUCCAUCACC
AACAAUUCUACAGGCAUGGAGGGCGAGAUCAAGAAUUGUUCUUAUAACAUCACCACAGAGCUGC
GCGACAAGAGGCAGAAAGUGUACAGCCUGUUCUAUCGCCUGGAUGUGGUGCAGAUCAAUGACU
CUAACGAUCGCAACAAUAGCCAGUACAGGCUGAUCAAUUGCAACACCACAACCAUGACCCAGGCC
UGUCCUAAGGUGACAUUUGACCCUAUCCCAAUCCACUAUUGCGCCCCAGCCGGCUUCGCCAUCC
UGAAGUGUAACAAUAAGACCUUUAAUGGCAAGGGCCCCUGCAACAAUGUGAGCUCCGUGCAGU
GUACCCACGGCAUCAAGCCUGUGGUGUCUACACAGCUGCUGCUGAACGGCAGCCUGGCCGAGAA
GGAGAUCAUCAUCAGGAGCGAGAAUCUGACCGACAACGUGAAGACAAUCAUCGUGCACCUGAAU
GAGAGCGUGGAGAUCAACUGCACCAGACCAAACAAUAACACAGUGAAGUCCGUGCGGAUCGGAC
CAGGACAGACCUUCUACUAUACAGGCGAUAUCAUCGGCAAUAUCCGCCAGGCCCACUGUACCGU
GAAUAAGACAGAGUGGAACACAGCCCUGACCAGGGUGAGCAAGAAGCUGAAGGAGUACUUCCCC
AACAAGACCAUCGCCUUUCAGCCUUCUAGCGGCGGCGACCUGGAGAUCACAACCUUCUCCUUUA
AUUGCAGAGGCGAGUUCUUUUAUUGUAACACAUCCGAUCUGUUCAAUGGCACCUUUAACGAGA
CAUCUGGCCAGUUCAAUUCCACCUUUAACUCUACACUGCAGUGCCGGAUCAAGCAGAUCAUCAA
UAUGUGGCAGGAAGUGGGACAGGCAAUGUACGCCCCUCCCAUCGCAGGCAGCAUCACCUGUAUC
UCCAACAUCACCGGCCUGAUCCUGACACGCGACGGCGGAAAUACAAACUCCACCAAGGAGACAUU
CAGGCCUGGCGGCGGCAAUAUGAGAGAUAACUGGCGGUCUGAGCUGUACAAGUAUAAGGUGGU
GAAGAUCGAGCCACUGGGAGUGGCACCAACCAAGUGCAGGAGACGGGUGGUGGGCAGCCACUC
CGGCUCUGGCGGCAGCGGCUCCGGCGGCCACGCAGCAGUGGGCAUCGGCGCCGUGUCUAUCGG
CUUUCUGGGAGCAGCAGGCUCCACCAUGGGAGCAGCCUCUAUCACACUGACCGUGCAGGCCAGA
CAGCUGCUGAGCGGCAUCGUGCAGCAGCAGUCCAACCUGCUGAGGGCACCAGAGCCUCAGCAGC
ACCUGCUGAAGGACACCCACUGGGGCAUCAAGCAGCUGCAGGCCAGGGUGCUGGCAGUGGAGCA
CUACCUGAAGGAUCAGCAGCUGCUGGGCAUCUGGGGCUGUAGCGGCAAGCUGAUCUGCUGUAC
AAAUGUGCCCUGGAACUCCUCUUGGUCUAACAAGAGCCAGGACGAGAUCUGGGAUAAUAUGAC
CUGGCUGAACUGGAGCAAGGAGAUCUCCAAUUACACACAGAUCAUCUAUAACCUGAUUGAAGAA
UCACAGACUCAGCAGGAACUGAAUAAUAGGUCACUGCUGGCACUGGAUUGAUAA (SEQ ID
NO: 191)
CE0217_FJ443575_MD39_L14G8 (amino acid, dna, rna)
MDWTWILFLVAAATRVHSAKDMWVTVYYGVPVWREAKTTLFCASDAKAYEREVHNVWATHACVPTDP
NPQERVLENVTENFNMWKNNMVDQMHEDIISLWDESLKPCIKLTPLCVTLNCGNAIVNESTIEGMKNCS
FNVTTELKDKKKKEYALFYKLDVVPLNGENNNSNSKNFSEYRLINCNTSTITQACPKVSFDPIPIHYCAPAGF
AILKCNNETFNGTGPCNNVSTVQCTHGIKPVVSTQLLLNGSLAEKEIIIRSENLTNNAKIIIVHLNNPVKIICTR
PGNNTVKSMRIGPGQTFYYTGDIIGDIRRAYCNISEKTWYDTLKNVSDKFQEHFPNASIEFKPSAGGDLEIT
THSFNCRGEFFYCDTSELFNGTYNNSTYNSSNNITLQCKIKQIINMWQGVGRAMYAPPIAGNITCESNITG
LLLTRDGGNNKSTPETFRPGGGDMRDNWRSELYKYKVVEIKPLGIAPTKCKRRVVGSHSGSGGSGSGGHA
AVGMGAVSLGFLGAAGSTMGAASLTLTVQARQLLSGIVQQQNNLLRAPEPQQHMLQDTHWGIKQLQA
RVLAIEHYLTDQQLLGIWGCSGKLICCTNVPWNNSWSNKSYEDIWGRNMTWMNWSREINNYTNTIYRL
LEKSQNQQEKNNKSLLELD** (SEQ ID NO: 195)
atggactggacttggattctgtttctggtcgccgccgcaactcgcgtgcattcagcaaaagatatgtgggtcaccgtctattatggagt
gcccgtgtggcgggaggccaagaccacactgttttgcgcaagcgacgcaaaggcatacgagagggaggtgcacaacgtgtgggcc
acacacgcctgcgtgccaaccgatccaaatccccaggagagagtgctggagaacgtgaccgagaatttcaacatgtggaagaaca
atatggtggaccagatgcacgaggatatcatctctctgtgggacgagagcctgaagccctgcatcaagctgacacctctgtgcgtga
ccctgaattgtggcaacgccatcgtgaatgagtccaccatcgagggcatgaagaattgttcttttaacgtgaccacagagctgaagg
acaagaagaagaaggagtacgccctgttctataagctggatgtggtgcccctgaacggcgagaacaacaactctaacagcaagaa
ctttagcgagtacaggctgatcaattgcaacacctccacaatcacccaggcctgtcccaaggtgtctttcgatcctatcccaatccact
attgcgcccctgccggcttcgccatcctgaagtgtaataacgagacattcaacggcaccggcccatgcaataacgtgtccacagtgc
agtgtacccacggcatcaagcccgtggtgtctacacagctgctgctgaatggcagcctggccgagaaggagatcatcatcaggtctg
agaacctgaccaataacgccaagatcatcatcgtgcacctgaataacccagtgaagatcatctgcacaaggcccggcaataacacc
gtgaagagcatgagaatcggccctggccagacattctactataccggcgacatcatcggcgatatcaggagagcctactgtaacatc
tctgagaagacatggtatgacaccctgaagaatgtgagcgataagttccaggagcactttcctaacgcctccatcgagttcaagccat
ctgccggcggcgacctggagatcaccacacactcctttaattgcaggggcgagttcttttactgtgatacaagcgagctgttcaatgg
cacatacaataactccacctataacagctccaataacatcaccctgcagtgcaagatcaagcagatcatcaacatgtggcagggcgt
gggcagagccatgtatgcccctcccatcgccggcaatatcacctgtgagagcaacatcacaggcctgctgctgacccgggacggcg
gaaataacaagtccacaccagagacattcaggcccggcggcggcgacatgagggataactggagaagcgagctgtacaagtataa
ggtggtggagatcaagcctctgggcatcgccccaacaaagtgcaagaggagggtggtgggctcccactctggcagcggcggctccg
gctctggcggccacgcagccgtgggcatgggcgccgtgtctctgggcttcctgggagcagcaggcagcaccatgggagcagcatcc
ctgacactgaccgtgcaggcaaggcagctgctgagcggcatcgtgcagcagcagaataacctgctgagagcccccgagcctcagca
gcacatgctgcaggacacacactggggcatcaagcagctgcaggcccgggtgctggcaatcgagcactacctgacagatcagcag
ctgctgggcatctggggctgttccggcaagctgatctgctgtaccaatgtgccctggaataacagctggtccaacaagtcctatgagg
atatctggggccggaatatgacctggatgaactggagcagggagatcaacaactacacaaacaccatctatcgcctgctggaaaag
tcacagaatcagcaggagaagaataataagtcactgctggaactggactgataa (SEQ ID NO: 193)
AUGGACUGGACUUGGAUUCUGUUUCUGGUCGCCGCCGCAACUCGCGUGCAUUCAGCAAAAGAU
AUGUGGGUCACCGUCUAUUAUGGAGUGCCCGUGUGGCGGGAGGCCAAGACCACACUGUUUUGC
GCAAGCGACGCAAAGGCAUACGAGAGGGAGGUGCACAACGUGUGGGCCACACACGCCUGCGUGC
CAACCGAUCCAAAUCCCCAGGAGAGAGUGCUGGAGAACGUGACCGAGAAUUUCAACAUGUGGAA
GAACAAUAUGGUGGACCAGAUGCACGAGGAUAUCAUCUCUCUGUGGGACGAGAGCCUGAAGCC
CUGCAUCAAGCUGACACCUCUGUGCGUGACCCUGAAUUGUGGCAACGCCAUCGUGAAUGAGUC
CACCAUCGAGGGCAUGAAGAAUUGUUCUUUUAACGUGACCACAGAGCUGAAGGACAAGAAGAA
GAAGGAGUACGCCCUGUUCUAUAAGCUGGAUGUGGUGCCCCUGAACGGCGAGAACAACAACUC
UAACAGCAAGAACUUUAGCGAGUACAGGCUGAUCAAUUGCAACACCUCCACAAUCACCCAGGCC
UGUCCCAAGGUGUCUUUCGAUCCUAUCCCAAUCCACUAUUGCGCCCCUGCCGGCUUCGCCAUCC
UGAAGUGUAAUAACGAGACAUUCAACGGCACCGGCCCAUGCAAUAACGUGUCCACAGUGCAGUG
UACCCACGGCAUCAAGCCCGUGGUGUCUACACAGCUGCUGCUGAAUGGCAGCCUGGCCGAGAAG
GAGAUCAUCAUCAGGUCUGAGAACCUGACCAAUAACGCCAAGAUCAUCAUCGUGCACCUGAAUA
ACCCAGUGAAGAUCAUCUGCACAAGGCCCGGCAAUAACACCGUGAAGAGCAUGAGAAUCGGCCC
UGGCCAGACAUUCUACUAUACCGGCGACAUCAUCGGCGAUAUCAGGAGAGCCUACUGUAACAUC
UCUGAGAAGACAUGGUAUGACACCCUGAAGAAUGUGAGCGAUAAGUUCCAGGAGCACUUUCCU
AACGCCUCCAUCGAGUUCAAGCCAUCUGCCGGCGGCGACCUGGAGAUCACCACACACUCCUUUA
AUUGCAGGGGCGAGUUCUUUUACUGUGAUACAAGCGAGCUGUUCAAUGGCACAUACAAUAACU
CCACCUAUAACAGCUCCAAUAACAUCACCCUGCAGUGCAAGAUCAAGCAGAUCAUCAACAUGUGG
CAGGGCGUGGGCAGAGCCAUGUAUGCCCCUCCCAUCGCCGGCAAUAUCACCUGUGAGAGCAACA
UCACAGGCCUGCUGCUGACCCGGGACGGCGGAAAUAACAAGUCCACACCAGAGACAUUCAGGCC
CGGCGGCGGCGACAUGAGGGAUAACUGGAGAAGCGAGCUGUACAAGUAUAAGGUGGUGGAGA
UCAAGCCUCUGGGCAUCGCCCCAACAAAGUGCAAGAGGAGGGUGGUGGGCUCCCACUCUGGCAG
CGGCGGCUCCGGCUCUGGCGGCCACGCAGCCGUGGGCAUGGGCGCCGUGUCUCUGGGCUUCCU
GGGAGCAGCAGGCAGCACCAUGGGAGCAGCAUCCCUGACACUGACCGUGCAGGCAAGGCAGCUG
CUGAGCGGCAUCGUGCAGCAGCAGAAUAACCUGCUGAGAGCCCCCGAGCCUCAGCAGCACAUGC
UGCAGGACACACACUGGGGCAUCAAGCAGCUGCAGGCCCGGGUGCUGGCAAUCGAGCACUACCU
GACAGAUCAGCAGCUGCUGGGCAUCUGGGGCUGUUCCGGCAAGCUGAUCUGCUGUACCAAUGU
GCCCUGGAAUAACAGCUGGUCCAACAAGUCCUAUGAGGAUAUCUGGGGCCGGAAUAUGACCUG
GAUGAACUGGAGCAGGGAGAUCAACAACUACACAAACACCAUCUAUCGCCUGCUGGAAAAGUCA
CAGAAUCAGCAGGAGAAGAAUAAUAAGUCACUGCUGGAACUGGACUGAUAA (SEQ ID NO: 194)
CE1176_FJ444437_MD39_L14G8 (amino acid, dna, rna)
MDWTWILFLVAAATRVHSVGNLWVTVYYGVPVWKEAKTTLFCASDAKAYEKEVHNVWATHACVPTDP
NPQEMVLENVTENFNMWKNDMVDQMHEDVISLWDQSLKPCVKLTPLCVTLTCTNTTVSNGSSNSNAN
FEEMKNCSFNATTEIKDKKKNEYALFYKLDIVPLNNSSGKYRLINCNTSAIAQACPKVTFEPIPIHYCAPAGYA
ILKCNNKTFNGTGPCNNVSTVQCTHGIKPVVSTQLLLNGSLAEKEIIIRSENLTNNAKTIIIHLNESVGIVCTRP
SNNTVKSIRIGPGQTFYYTGDIIGDIRQAHCNVSKQNWNRTLQQVGRKLAEHFPNRNITFAHSSGGDLEIT
THSFNCRGEFFYCNTSGLFNGTYHPNGTYNETAVNSSDTITLQCRIKQIINMWQEVGRAMYAPPIAGNITC
NSTITGLLLTRDGGINQTGEEIFRPGGGDMRDNWRNELYKYKVVEIKPLGIAPTKCKRRVVGSHSGSGGSG
SGGHAAVGIGAVSLGFLGAAGSTMGAASITLTVQARQLLSGIVQQQSNLLRAPEPQQHMLQDTHWGIK
QLQARVLAIEHYLKDQQLLGIWGCSGKLICCTNVPWNSSWSNRSQEDIWNNMTWMNWSREIDNYTHT
IYSLLEESQIQQEKNNKSLLALD** (SEQ ID NO: 198)
atggattggacttggattctgtttctggtcgccgccgctactcgcgtgcattcagtgggcaacctgtgggtcaccgtctactatggggtg
cccgtgtggaaggaggccaagaccacactgttctgcgcctccgacgccaaggcctacgagaaggaggtgcacaacgtgtgggcca
cacacgcctgcgtgcctaccgatccaaatccccaggagatggtgctggagaacgtgacagagaactttaatatgtggaagaacgac
atggtggatcagatgcacgaggacgtgatctctctgtgggatcagagcctgaagccttgcgtgaagctgaccccactgtgcgtgacc
ctgacatgtaccaataccacagtgtccaacggcagctccaactctaatgccaacttcgaggagatgaagaattgttcttttaacgcca
ccacagagatcaaggacaagaagaagaacgagtacgccctgttctataagctggatatcgtgcccctgaacaattctagcggcaag
tataggctgatcaattgcaacacaagcgccatcgcccaggcctgtccaaaggtgaccttcgagcctatcccaatccactactgcgccc
ccgccggctatgccatcctgaagtgtaacaacaagaccttcaacggcaccggcccttgcaacaacgtgagcacagtgcagtgtaccc
acggcatcaagccagtggtgagcacccagctgctgctgaacggctccctggcagagaaggagatcatcatccggagcgagaatctg
acaaacaatgccaagaccatcatcatccacctgaacgagtccgtgggcatcgtgtgcacacggcccagcaacaataccgtgaagtc
catccgcatcggccctggccagaccttctactataccggcgacatcatcggcgatatccgccaggcccactgtaatgtgagcaagca
gaattggaacaggacactgcagcaagtgggcagaaagctggccgagcacttcccaaataggaacatcacctttgcccactcctctg
gcggcgacctggagatcaccacacactccttcaactgcagaggcgagttcttttactgtaatacatctggcctgtttaacggcacctac
caccccaatggcacatataacgagacagccgtgaatagctccgatacaatcaccctgcagtgcaggatcaagcagatcatcaacat
gtggcaggaagtgggcagagccatgtatgcccctcccatcgccggcaatatcacctgtaacagcacaatcaccggcctgctgctgac
acgggacggcggcatcaaccagaccggagaggagatcttccgccccggcggcggcgacatgcgggataattggcgcaacgagctg
tacaagtataaggtggtggagatcaagccactgggcatcgcccccacaaagtgcaagaggagagtggtgggctcccactctggcag
cggcggctccggctctggcggccacgcagccgtgggcatcggagccgtgtccctgggctttctgggagcagcaggctctaccatggg
agcagccagcatcacactgaccgtgcaggcaaggcagctgctgtccggcatcgtgcagcagcagtctaacctgctgagagcccccg
agcctcagcagcacatgctgcaggacacccactggggcatcaagcagctgcaggccagggtgctggccatcgagcactacctgaag
gatcagcagctgctgggcatctggggctgttctggcaagctgatctgctgtacaaatgtgccatggaactctagctggagcaaccggt
cccaggaggacatctggaacaatatgacctggatgaattggagcagggagatcgataactacacacacaccatctatagcctgctg
gaggagtcacagattcagcaggagaaaaataataagtcactgctggcactggactgataa (SEQ ID NO: 196)
AUGGAUUGGACUUGGAUUCUGUUUCUGGUCGCCGCCGCUACUCGCGUGCAUUCAGUGGGCAA
CCUGUGGGUCACCGUCUACUAUGGGGUGCCCGUGUGGAAGGAGGCCAAGACCACACUGUUCUG
CGCCUCCGACGCCAAGGCCUACGAGAAGGAGGUGCACAACGUGUGGGCCACACACGCCUGCGUG
CCUACCGAUCCAAAUCCCCAGGAGAUGGUGCUGGAGAACGUGACAGAGAACUUUAAUAUGUGG
AAGAACGACAUGGUGGAUCAGAUGCACGAGGACGUGAUCUCUCUGUGGGAUCAGAGCCUGAAG
CCUUGCGUGAAGCUGACCCCACUGUGCGUGACCCUGACAUGUACCAAUACCACAGUGUCCAACG
GCAGCUCCAACUCUAAUGCCAACUUCGAGGAGAUGAAGAAUUGUUCUUUUAACGCCACCACAGA
GAUCAAGGACAAGAAGAAGAACGAGUACGCCCUGUUCUAUAAGCUGGAUAUCGUGCCCCUGAAC
AAUUCUAGCGGCAAGUAUAGGCUGAUCAAUUGCAACACAAGCGCCAUCGCCCAGGCCUGUCCAA
AGGUGACCUUCGAGCCUAUCCCAAUCCACUACUGCGCCCCCGCCGGCUAUGCCAUCCUGAAGUG
UAACAACAAGACCUUCAACGGCACCGGCCCUUGCAACAACGUGAGCACAGUGCAGUGUACCCACG
GCAUCAAGCCAGUGGUGAGCACCCAGCUGCUGCUGAACGGCUCCCUGGCAGAGAAGGAGAUCAU
CAUCCGGAGCGAGAAUCUGACAAACAAUGCCAAGACCAUCAUCAUCCACCUGAACGAGUCCGUG
GGCAUCGUGUGCACACGGCCCAGCAACAAUACCGUGAAGUCCAUCCGCAUCGGCCCUGGCCAGA
CCUUCUACUAUACCGGCGACAUCAUCGGCGAUAUCCGCCAGGCCCACUGUAAUGUGAGCAAGCA
GAAUUGGAACAGGACACUGCAGCAAGUGGGCAGAAAGCUGGCCGAGCACUUCCCAAAUAGGAAC
AUCACCUUUGCCCACUCCUCUGGCGGCGACCUGGAGAUCACCACACACUCCUUCAACUGCAGAG
GCGAGUUCUUUUACUGUAAUACAUCUGGCCUGUUUAACGGCACCUACCACCCCAAUGGCACAUA
UAACGAGACAGCCGUGAAUAGCUCCGAUACAAUCACCCUGCAGUGCAGGAUCAAGCAGAUCAUC
AACAUGUGGCAGGAAGUGGGCAGAGCCAUGUAUGCCCCUCCCAUCGCCGGCAAUAUCACCUGUA
ACAGCACAAUCACCGGCCUGCUGCUGACACGGGACGGCGGCAUCAACCAGACCGGAGAGGAGAU
CUUCCGCCCCGGCGGCGGCGACAUGCGGGAUAAUUGGCGCAACGAGCUGUACAAGUAUAAGGU
GGUGGAGAUCAAGCCACUGGGCAUCGCCCCCACAAAGUGCAAGAGGAGAGUGGUGGGCUCCCAC
UCUGGCAGCGGCGGCUCCGGCUCUGGCGGCCACGCAGCCGUGGGCAUCGGAGCCGUGUCCCUG
GGCUUUCUGGGAGCAGCAGGCUCUACCAUGGGAGCAGCCAGCAUCACACUGACCGUGCAGGCAA
GGCAGCUGCUGUCCGGCAUCGUGCAGCAGCAGUCUAACCUGCUGAGAGCCCCCGAGCCUCAGCA
GCACAUGCUGCAGGACACCCACUGGGGCAUCAAGCAGCUGCAGGCCAGGGUGCUGGCCAUCGAG
CACUACCUGAAGGAUCAGCAGCUGCUGGGCAUCUGGGGCUGUUCUGGCAAGCUGAUCUGCUGU
ACAAAUGUGCCAUGGAACUCUAGCUGGAGCAACCGGUCCCAGGAGGACAUCUGGAACAAUAUGA
CCUGGAUGAAUUGGAGCAGGGAGAUCGAUAACUACACACACACCAUCUAUAGCCUGCUGGAGGA
GUCACAGAUUCAGCAGGAGAAAAAUAAUAAGUCACUGCUGGCACUGGACUGAUAA (SEQ ID
NO: 197)
25710_EF117271_MD39_L14G8 (amino acid, dna, rna)
MDWTWILFLVAAATRVHSGGNLWVTVYYGVPVWKEATTTLFCASDAKAYDKEVHNVWATHACVPTDP
NPQEMVLGNVTENFNMWKNEMVNQMHEDVISLWDQSLKPCVKLTPLCVTLECSNVTYNESMKEVKN
CSFNLTTELRDKKQKVHALFYRLDIVPLNDTEKKNSSRPYRLINCNTSAITQACPKVTFDPIPIHYCTPAGYAIL
KCNDKKFNGTGPCHKVSTVQCTHGIKPVVSTQLLLNGSLAEGEIIIRSENLTNNAKTIIVHLNQSVEIVCARP
SNNTVTSIRIGPGQTFYYTGAITGDIRQAHCNISKDKWNETLQRVGEKLAEHFPNKTIKFASSSGGDLEITTH
SFNCRGEFFYCNTSGLFNGTFNGTYVSPNSTDSNSSSIITIPCRIKQIINMWQEVGRAMYAPPIAGNITCKS
NITGLLLVRDGGTGSESNKTEIFRPGGGDMRDNWRSELYKYKVVEIKPLGVAPTKCKRRVVGSHSGSGGS
GSGGHAAVGIGAVSLGFLGAAGSTMGAASITLTVQARQLLSGIVQQQSNLLRAPEPQQHLLQDTHWGIK
QLQTRVLAIEHYLKDQQLLGIWGCSGKLICCTAVPWNYSWSNRSQDDIWDNMTWMNWSKEISNYTNTI
YKLLEDSQIQQEKNNKSLLALD** (SEQ ID NO: 201)
atggactggacttggattctgttcctggtcgccgccgctactcgcgtgcattctgggggcaacctgtgggtcaccgtgtattatggagtg
cccgtgtggaaggaggccaccacaaccctgttctgcgccagcgacgccaaggcctacgataaggaggtgcacaacgtgtgggcaa
cccacgcatgcgtgccaacagacccaaacccccaggagatggtgctgggcaatgtgaccgagaactttaatatgtggaagaacgag
atggtgaatcagatgcacgaggacgtgatctccctgtgggatcagtctctgaagccttgcgtgaagctgaccccactgtgcgtgacac
tggagtgttccaacgtgacctataatgagtctatgaaggaggtgaagaactgttccttcaatctgacaaccgagctgagggataaga
agcagaaggtgcacgccctgttttacagactggacatcgtgcccctgaacgataccgagaagaagaatagctcccggccttatcgcc
tgatcaactgcaatacaagcgccatcacccaggcctgtcctaaggtgaccttcgaccctatcccaatccactactgcacaccagccgg
ctatgccatcctgaagtgtaacgataagaagtttaatggcaccggcccatgccacaaggtgtccacagtgcagtgtacccacggcat
caagcccgtggtgtctacacagctgctgctgaacggcagcctggcagagggcgagatcatcatcaggagcgagaacctgaccaaca
atgccaagacaatcatcgtgcacctgaatcagtccgtggagatcgtgtgcgcccggccaagcaacaatacagtgacctccatcagga
tcggaccaggacagacattctactataccggcgccatcacaggcgacatcaggcaggcccactgtaacatcagcaaggataagtgg
aatgagacactgcagagagtgggcgagaagctggccgagcacttccccaacaagacaatcaagtttgcctctagctccggcggcga
cctggagatcacaacccactcctttaactgcaggggcgagttcttttactgtaatacctctggcctgttcaacggcacctttaatggcac
atacgtgagccccaacagcaccgattccaattctagctccatcatcacaatcccttgccggatcaagcagatcatcaatatgtggcag
gaagtgggaagggcaatgtacgcccctcccatcgccggcaacatcacctgtaagtccaatatcacaggcctgctgctggtgagggac
ggcggaaccggctctgagagcaacaagacagagatcttcagacccggcggcggcgacatgagggataattggagatctgagctgt
acaagtataaggtggtggagatcaagccactgggcgtggcccccaccaagtgcaagaggagagtggtgggctcccactctggcagc
ggcggctccggctctggcggccacgcagccgtgggcatcggagccgtgtccctgggctttctgggagcagcaggctctacaatggga
gcagccagcatcacactgaccgtgcaggcaaggcagctgctgagcggcatcgtgcagcagcagtccaacctgctgagggcaccag
agcctcagcagcacctgctgcaggacacccactggggcatcaagcagctgcagacacgggtgctggccatcgagcactacctgaag
gatcagcagctgctgggcatctggggctgttctggcaagctgatctgctgtaccgccgtgccctggaactatagctggtccaatcgca
gccaggacgatatctgggacaacatgacatggatgaattggtctaaggagatcagcaactacacaaataccatctataagctgctgg
aagatagtcagattcagcaggaaaagaacaataagtcactgctggcactggattgataa (SEQ ID NO: 199)
AUGGACUGGACUUGGAUUCUGUUCCUGGUCGCCGCCGCUACUCGCGUGCAUUCUGGGGGCAAC
CUGUGGGUCACCGUGUAUUAUGGAGUGCCCGUGUGGAAGGAGGCCACCACAACCCUGUUCUGC
GCCAGCGACGCCAAGGCCUACGAUAAGGAGGUGCACAACGUGUGGGCAACCCACGCAUGCGUGC
CAACAGACCCAAACCCCCAGGAGAUGGUGCUGGGCAAUGUGACCGAGAACUUUAAUAUGUGGAA
GAACGAGAUGGUGAAUCAGAUGCACGAGGACGUGAUCUCCCUGUGGGAUCAGUCUCUGAAGCC
UUGCGUGAAGCUGACCCCACUGUGCGUGACACUGGAGUGUUCCAACGUGACCUAUAAUGAGUC
UAUGAAGGAGGUGAAGAACUGUUCCUUCAAUCUGACAACCGAGCUGAGGGAUAAGAAGCAGAA
GGUGCACGCCCUGUUUUACAGACUGGACAUCGUGCCCCUGAACGAUACCGAGAAGAAGAAUAGC
UCCCGGCCUUAUCGCCUGAUCAACUGCAAUACAAGCGCCAUCACCCAGGCCUGUCCUAAGGUGA
CCUUCGACCCUAUCCCAAUCCACUACUGCACACCAGCCGGCUAUGCCAUCCUGAAGUGUAACGAU
AAGAAGUUUAAUGGCACCGGCCCAUGCCACAAGGUGUCCACAGUGCAGUGUACCCACGGCAUCA
AGCCCGUGGUGUCUACACAGCUGCUGCUGAACGGCAGCCUGGCAGAGGGCGAGAUCAUCAUCA
GGAGCGAGAACCUGACCAACAAUGCCAAGACAAUCAUCGUGCACCUGAAUCAGUCCGUGGAGAU
CGUGUGCGCCCGGCCAAGCAACAAUACAGUGACCUCCAUCAGGAUCGGACCAGGACAGACAUUC
UACUAUACCGGCGCCAUCACAGGCGACAUCAGGCAGGCCCACUGUAACAUCAGCAAGGAUAAGU
GGAAUGAGACACUGCAGAGAGUGGGCGAGAAGCUGGCCGAGCACUUCCCCAACAAGACAAUCAA
GUUUGCCUCUAGCUCCGGCGGCGACCUGGAGAUCACAACCCACUCCUUUAACUGCAGGGGCGAG
UUCUUUUACUGUAAUACCUCUGGCCUGUUCAACGGCACCUUUAAUGGCACAUACGUGAGCCCC
AACAGCACCGAUUCCAAUUCUAGCUCCAUCAUCACAAUCCCUUGCCGGAUCAAGCAGAUCAUCAA
UAUGUGGCAGGAAGUGGGAAGGGCAAUGUACGCCCCUCCCAUCGCCGGCAACAUCACCUGUAAG
UCCAAUAUCACAGGCCUGCUGCUGGUGAGGGACGGCGGAACCGGCUCUGAGAGCAACAAGACAG
AGAUCUUCAGACCCGGCGGCGGCGACAUGAGGGAUAAUUGGAGAUCUGAGCUGUACAAGUAUA
AGGUGGUGGAGAUCAAGCCACUGGGCGUGGCCCCCACCAAGUGCAAGAGGAGAGUGGUGGGCU
CCCACUCUGGCAGCGGCGGCUCCGGCUCUGGCGGCCACGCAGCCGUGGGCAUCGGAGCCGUGUC
CCUGGGCUUUCUGGGAGCAGCAGGCUCUACAAUGGGAGCAGCCAGCAUCACACUGACCGUGCAG
GCAAGGCAGCUGCUGAGCGGCAUCGUGCAGCAGCAGUCCAACCUGCUGAGGGCACCAGAGCCUC
AGCAGCACCUGCUGCAGGACACCCACUGGGGCAUCAAGCAGCUGCAGACACGGGUGCUGGCCAU
CGAGCACUACCUGAAGGAUCAGCAGCUGCUGGGCAUCUGGGGCUGUUCUGGCAAGCUGAUCUG
CUGUACCGCCGUGCCCUGGAACUAUAGCUGGUCCAAUCGCAGCCAGGACGAUAUCUGGGACAAC
AUGACAUGGAUGAAUUGGUCUAAGGAGAUCAGCAACUACACAAAUACCAUCUAUAAGCUGCUG
GAAGAUAGUCAGAUUCAGCAGGAAAAGAACAAUAAGUCACUGCUGGCACUGGAUUGAUAA (SEQ
ID NO: 200)
BJOX2000_HM215364_MD39_L14G8 (amino acid, dna, rna)
MDWTWILFLVAAATRVHSVGNLWVTVYYGVPVWKEATTTLFCASDAKAYDTEVHNVWATHACVPTDP
DPQEMFLENVTENFNMWKNNMVDQMHEDVISLWDQSLKPCVKLTPLCVTLECKNVNSSSSDTKNGTD
PEMKNCSFNATTELRDRKQKVYALFYKLDIVPLNEKNSSEYRLINCNTSTITQACPKVTFDPIPIHYCTPAGYA
ILKCNDEKFNGTGPCSNVSTVQCTHGIKPVVSTQLLLNGSLAEKGIIIRSENLTNNVKTIIVHLNQSVEILCIRP
NNNTVKSIRIGPGQTFYYTGEIIGDIRQAHCNISGKVWNETLQRVGEKLAEYFPNKTIKFASSSGGDLEITTH
SFNCGGEFFYCNTSKLFNGTFNGTYMPNVTEGNSTISIPCRIKQIINMWQKVGRAMYAPPIEGNITCKSKIT
GLLLERDGGPENDTEIFRPGGGDMRNNWRSELYKYKVVEIKPLGVAPTECKRRVVGSHSGSGGSGSGGH
AAVGIGAVSLGFLGVAGSTMGAASMALTVQARQLLSGIVQQQSNLLRAPEPQQHLLQDTHWGIKQLQT
RVLAIEHYLKDQQLLGIWGCSGKLICCTAVPWNSSWSNKSQEEIWENMTWMNWSKEISNYTDTIYRLLE
DSQNQQERNNKSLLALD** (SEQ ID NO: 204)
atggactggacttggattctgtttctggtcgcagcagcaactcgggtgcatagcgtcggcaacctgtgggtcactgtctactacggggt
gcccgtgtggaaggaggccaccacaaccctgttctgcgccagcgacgccaaggcctacgataccgaggtgcacaacgtgtgggcaa
cccacgcatgcgtgcctacagacccagatccccaggagatgttcctggagaacgtgacagagaacttcaacatgtggaagaacaat
atggtggaccagatgcacgaggatgtgatcagcctgtgggaccagtccctgaagccttgcgtgaagctgaccccactgtgcgtgaca
ctggagtgtaagaatgtgaacagctcctctagcgacaccaagaacggcacagatcctgagatgaagaattgttctttcaacgccaca
accgagctgcgggaccgcaagcagaaggtgtacgccctgttttataagctggatatcgtgccactgaatgagaagaactcctctgag
tatcggctgatcaattgcaacacaagcaccatcacacaggcctgtcccaaggtgaccttcgaccctatcccaatccactactgcacac
ctgccggctatgccatcctgaagtgtaatgatgagaagtttaacggcaccggcccatgctccaacgtgagcaccgtgcagtgtacac
acggcatcaagcccgtggtgagcacacagctgctgctgaacggctccctggccgagaagggcatcatcatccgctccgagaatctg
accaacaatgtgaagacaatcatcgtgcacctgaaccagtccgtggagatcctgtgcatccggccaaacaataacaccgtgaagtct
atccgcatcggccccggccagaccttctactatacaggcgagatcatcggcgacatccggcaggcccactgtaatatctctggcaag
gtctggaacgagacactgcagagggtgggagagaagctggcagagtacttcccaaacaagacaatcaagtttgccagctcctctgg
cggcgatctggagatcacaacccactcttttaattgcggcggcgagttcttttactgtaacaccagcaagctgttcaatggcaccttta
acggcacatatatgcctaatgtgaccgagggcaacagcacaatctccatcccatgccggatcaagcagatcatcaatatgtggcaga
aagtgggccgcgccatgtatgcccctcccatcgagggcaacatcacctgtaagagcaagatcacaggcctgctgctggagagggac
ggcggaccagagaacgataccgagatcttcagacccggcggcggcgacatgaggaataactggagatccgagctgtacaagtata
aggtggtggagatcaagccactgggagtggcaccaaccgagtgcaagaggagagtggtgggctctcacagcggctccggcggctct
ggcagcggcggccacgccgccgtgggcatcggagccgtgagcctgggctttctgggagtggcaggctctaccatgggagcagcaag
catggcactgacagtgcaggccaggcagctgctgtccggcatcgtgcagcagcagtctaatctgctgagagcaccagagcctcagc
agcacctgctgcaggacacccactggggcatcaagcagctgcagacaagggtgctggccatcgagcactacctgaaggatcagca
gctgctgggcatctggggctgttccggcaagctgatctgctgtaccgccgtgccttggaatagctcctggtctaacaagagccaggag
gagatctgggagaatatgacatggatgaactggtccaaggagatctctaactacaccgatacaatctatagactgctggaagatagt
cagaatcagcaggagagaaataataagtcactgctggcactggattgataa (SEQ ID NO: 202)
AUGGACUGGACUUGGAUUCUGUUUCUGGUCGCAGCAGCAACUCGGGUGCAUAGCGUCGGCAAC
CUGUGGGUCACUGUCUACUACGGGGUGCCCGUGUGGAAGGAGGCCACCACAACCCUGUUCUGC
GCCAGCGACGCCAAGGCCUACGAUACCGAGGUGCACAACGUGUGGGCAACCCACGCAUGCGUGC
CUACAGACCCAGAUCCCCAGGAGAUGUUCCUGGAGAACGUGACAGAGAACUUCAACAUGUGGAA
GAACAAUAUGGUGGACCAGAUGCACGAGGAUGUGAUCAGCCUGUGGGACCAGUCCCUGAAGCC
UUGCGUGAAGCUGACCCCACUGUGCGUGACACUGGAGUGUAAGAAUGUGAACAGCUCCUCUAG
CGACACCAAGAACGGCACAGAUCCUGAGAUGAAGAAUUGUUCUUUCAACGCCACAACCGAGCUG
CGGGACCGCAAGCAGAAGGUGUACGCCCUGUUUUAUAAGCUGGAUAUCGUGCCACUGAAUGAG
AAGAACUCCUCUGAGUAUCGGCUGAUCAAUUGCAACACAAGCACCAUCACACAGGCCUGUCCCAA
GGUGACCUUCGACCCUAUCCCAAUCCACUACUGCACACCUGCCGGCUAUGCCAUCCUGAAGUGU
AAUGAUGAGAAGUUUAACGGCACCGGCCCAUGCUCCAACGUGAGCACCGUGCAGUGUACACACG
GCAUCAAGCCCGUGGUGAGCACACAGCUGCUGCUGAACGGCUCCCUGGCCGAGAAGGGCAUCAU
CAUCCGCUCCGAGAAUCUGACCAACAAUGUGAAGACAAUCAUCGUGCACCUGAACCAGUCCGUG
GAGAUCCUGUGCAUCCGGCCAAACAAUAACACCGUGAAGUCUAUCCGCAUCGGCCCCGGCCAGA
CCUUCUACUAUACAGGCGAGAUCAUCGGCGACAUCCGGCAGGCCCACUGUAAUAUCUCUGGCAA
GGUCUGGAACGAGACACUGCAGAGGGUGGGAGAGAAGCUGGCAGAGUACUUCCCAAACAAGAC
AAUCAAGUUUGCCAGCUCCUCUGGCGGCGAUCUGGAGAUCACAACCCACUCUUUUAAUUGCGG
CGGCGAGUUCUUUUACUGUAACACCAGCAAGCUGUUCAAUGGCACCUUUAACGGCACAUAUAU
GCCUAAUGUGACCGAGGGCAACAGCACAAUCUCCAUCCCAUGCCGGAUCAAGCAGAUCAUCAAU
AUGUGGCAGAAAGUGGGCCGCGCCAUGUAUGCCCCUCCCAUCGAGGGCAACAUCACCUGUAAGA
GCAAGAUCACAGGCCUGCUGCUGGAGAGGGACGGCGGACCAGAGAACGAUACCGAGAUCUUCAG
ACCCGGCGGCGGCGACAUGAGGAAUAACUGGAGAUCCGAGCUGUACAAGUAUAAGGUGGUGGA
GAUCAAGCCACUGGGAGUGGCACCAACCGAGUGCAAGAGGAGAGUGGUGGGCUCUCACAGCGG
CUCCGGCGGCUCUGGCAGCGGCGGCCACGCCGCCGUGGGCAUCGGAGCCGUGAGCCUGGGCUU
UCUGGGAGUGGCAGGCUCUACCAUGGGAGCAGCAAGCAUGGCACUGACAGUGCAGGCCAGGCA
GCUGCUGUCCGGCAUCGUGCAGCAGCAGUCUAAUCUGCUGAGAGCACCAGAGCCUCAGCAGCAC
CUGCUGCAGGACACCCACUGGGGCAUCAAGCAGCUGCAGACAAGGGUGCUGGCCAUCGAGCACU
ACCUGAAGGAUCAGCAGCUGCUGGGCAUCUGGGGCUGUUCCGGCAAGCUGAUCUGCUGUACCG
CCGUGCCUUGGAAUAGCUCCUGGUCUAACAAGAGCCAGGAGGAGAUCUGGGAGAAUAUGACAU
GGAUGAACUGGUCCAAGGAGAUCUCUAACUACACCGAUACAAUCUAUAGACUGCUGGAAGAUA
GUCAGAAUCAGCAGGAGAGAAAUAAUAAGUCACUGCUGGCACUGGAUUGAUAA (SEQ ID
NO: 203)
CH119_EF117261_MD39_L14G8 (amino acid, dna, rna)
MDWTWILFLVAAATRVHSVGNLWVTVYYGVPVWKEATTTLFCASDAKAYDTEVHNVWATHACVPTDP
SPQELVLENVTENFNMWKNEMVNQMHEDVISLWDQSLKPCVKLTPLCVTLECSKVSNNETDKYNGTEE
MKNCSFNATTVVRDRQQKVYALFYRLDIVPLTEKNSSENSSKYYRLINCNTSAITQACPKVSFEPIPIHYCTPA
GYAILKCNDKTFNGTGPCHNVSTVQCTHGIKPVVSTQLLLNGSLAEGEIIIRSENLTNNVKTILVHLNQSVEI
VCTRPNNNTVKSIRIGPGQTFYYTGDIIGDIRQAHCNISKWHETLKRVSEKLAEHFPNKTINFTSSSGGDLEIT
THSFTCRGEFFYCNTSGLFNSTYMPNGTYLHGDTNSNSSITIPCRIKQIINMWQEVGRAMYAPPIEGNITCK
SNITGLLLVRDGGTESNNTETNNTEIFRPGGGDMRDNWRSELYKYKVVEIKPLGVAPTACKRRVVGSHSG
SGGSGSGGHAAVGIGAVSLGFLGVAGSTMGAASMTLTVQARQLLSGIVQQQSNLLRAPEPQQHLLQDT
HWGIKQLQTRVLAIEHYLKDQQLLGIWGCSGKLICCTAVPWNSSWSNKSQKEIWDNMTWMNWSKEIS
NYTNTIYKLLEDSQNQQESNNKSLLALD** (SEQ ID NO: 207)
atggactggacttggattctgtttctggtcgcagccgcaactcgcgtgcattccgtgggcaacctgtgggtcaccgtctactatggggt
gccagtgtggaaggaggccaccacaaccctgttctgcgcctccgacgccaaggcctacgataccgaggtgcacaacgtgtgggcaa
cacacgcatgcgtgccaaccgacccatctccccaggagctggtgctggagaatgtgacagagaacttcaacatgtggaagaatgag
atggtgaaccagatgcacgaggacgtgatctccctgtgggatcagtctctgaagccttgcgtgaagctgacaccactgtgcgtgaccc
tggagtgttccaaggtgtctaacaatgagacagacaagtataacggcaccgaggagatgaagaattgtagcttcaacgcaacaacc
gtggtgcgggaccgccagcagaaggtgtacgccctgttttataggctggatatcgtgcccctgaccgagaagaatagctccgagaac
tctagcaagtactatagactgatcaattgcaacacatctgccatcacccaggcctgtccaaaggtgagcttcgagcctatcccaatcc
actactgcacccccgccggctatgccatcctgaagtgtaatgacaagaccttcaacggcaccggcccttgccacaacgtgagcacag
tgcagtgtacccacggcatcaagccagtggtgagcacacagctgctgctgaatggctccctggccgagggcgagatcatcatccggt
ccgagaacctgacaaacaatgtgaagaccatcctggtgcacctgaatcagagcgtggagatcgtgtgcacacggcccaacaataac
accgtgaagtccatccgcatcggccctggccagacattctactataccggcgacatcatcggcgatatccggcaggcccactgtaac
atctccaagtggcacgagacactgaagcgcgtgtctgagaagctggccgagcacttccctaataagacaatcaactttacctcctcta
gcggcggcgacctggagatcacaacccactctttcacctgccgcggcgagttcttttactgtaatacaagcggcctgtttaactccaca
tacatgcccaatggcacctatctgcacggcgatacaaattccaactcctctatcaccatcccttgcaggatcaagcagatcatcaaca
tgtggcaggaagtgggcagagccatgtatgcccctcccatcgagggcaacatcacctgtaagtctaatatcacaggcctgctgctggt
gcgggacggcggaaccgagagcaataacacagagacaaataacacagagatcttccgccccggcggcggcgacatgagggataa
ctggagaagcgagctgtacaagtataaggtggtggagatcaagccactgggagtggcaccaaccgcatgcaagaggagagtggtg
ggctctcacagcggctccggcggctctggcagcggcggccacgccgccgtgggcatcggagccgtgtccctgggctttctgggagtg
gcaggctctaccatgggagcagccagcatgacactgaccgtgcaggcaaggcagctgctgtccggcatcgtgcagcagcagtctaa
cctgctgagagcaccagagcctcagcagcacctgctgcaggacacccactggggcatcaagcagctgcagacacgggtgctggcc
atcgagcactacctgaaggatcagcagctgctgggcatctggggctgtagcggcaagctgatctgctgtaccgccgtgccttggaat
agctcctggagcaacaagtcccagaaggagatctgggataatatgacatggatgaactggtctaaggagatcagcaattacacaaa
caccatctataagctgctggaggactcacagaatcagcaggaatcaaacaacaaatccctgctggcactggactgataa (SEQ
ID NO: 205)
AUGGACUGGACUUGGAUUCUGUUUCUGGUCGCAGCCGCAACUCGCGUGCAUUCCGUGGGCAAC
CUGUGGGUCACCGUCUACUAUGGGGUGCCAGUGUGGAAGGAGGCCACCACAACCCUGUUCUGC
GCCUCCGACGCCAAGGCCUACGAUACCGAGGUGCACAACGUGUGGGCAACACACGCAUGCGUGC
CAACCGACCCAUCUCCCCAGGAGCUGGUGCUGGAGAAUGUGACAGAGAACUUCAACAUGUGGAA
GAAUGAGAUGGUGAACCAGAUGCACGAGGACGUGAUCUCCCUGUGGGAUCAGUCUCUGAAGCC
UUGCGUGAAGCUGACACCACUGUGCGUGACCCUGGAGUGUUCCAAGGUGUCUAACAAUGAGAC
AGACAAGUAUAACGGCACCGAGGAGAUGAAGAAUUGUAGCUUCAACGCAACAACCGUGGUGCG
GGACCGCCAGCAGAAGGUGUACGCCCUGUUUUAUAGGCUGGAUAUCGUGCCCCUGACCGAGAA
GAAUAGCUCCGAGAACUCUAGCAAGUACUAUAGACUGAUCAAUUGCAACACAUCUGCCAUCACC
CAGGCCUGUCCAAAGGUGAGCUUCGAGCCUAUCCCAAUCCACUACUGCACCCCCGCCGGCUAUG
CCAUCCUGAAGUGUAAUGACAAGACCUUCAACGGCACCGGCCCUUGCCACAACGUGAGCACAGU
GCAGUGUACCCACGGCAUCAAGCCAGUGGUGAGCACACAGCUGCUGCUGAAUGGCUCCCUGGCC
GAGGGCGAGAUCAUCAUCCGGUCCGAGAACCUGACAAACAAUGUGAAGACCAUCCUGGUGCACC
UGAAUCAGAGCGUGGAGAUCGUGUGCACACGGCCCAACAAUAACACCGUGAAGUCCAUCCGCAU
CGGCCCUGGCCAGACAUUCUACUAUACCGGCGACAUCAUCGGCGAUAUCCGGCAGGCCCACUGU
AACAUCUCCAAGUGGCACGAGACACUGAAGCGCGUGUCUGAGAAGCUGGCCGAGCACUUCCCUA
AUAAGACAAUCAACUUUACCUCCUCUAGCGGCGGCGACCUGGAGAUCACAACCCACUCUUUCAC
CUGCCGCGGCGAGUUCUUUUACUGUAAUACAAGCGGCCUGUUUAACUCCACAUACAUGCCCAAU
GGCACCUAUCUGCACGGCGAUACAAAUUCCAACUCCUCUAUCACCAUCCCUUGCAGGAUCAAGC
AGAUCAUCAACAUGUGGCAGGAAGUGGGCAGAGCCAUGUAUGCCCCUCCCAUCGAGGGCAACAU
CACCUGUAAGUCUAAUAUCACAGGCCUGCUGCUGGUGCGGGACGGCGGAACCGAGAGCAAUAAC
ACAGAGACAAAUAACACAGAGAUCUUCCGCCCCGGCGGCGGCGACAUGAGGGAUAACUGGAGAA
GCGAGCUGUACAAGUAUAAGGUGGUGGAGAUCAAGCCACUGGGAGUGGCACCAACCGCAUGCA
AGAGGAGAGUGGUGGGCUCUCACAGCGGCUCCGGCGGCUCUGGCAGCGGCGGCCACGCCGCCG
UGGGCAUCGGAGCCGUGUCCCUGGGCUUUCUGGGAGUGGCAGGCUCUACCAUGGGAGCAGCCA
GCAUGACACUGACCGUGCAGGCAAGGCAGCUGCUGUCCGGCAUCGUGCAGCAGCAGUCUAACCU
GCUGAGAGCACCAGAGCCUCAGCAGCACCUGCUGCAGGACACCCACUGGGGCAUCAAGCAGCUG
CAGACACGGGUGCUGGCCAUCGAGCACUACCUGAAGGAUCAGCAGCUGCUGGGCAUCUGGGGC
UGUAGCGGCAAGCUGAUCUGCUGUACCGCCGUGCCUUGGAAUAGCUCCUGGAGCAACAAGUCC
CAGAAGGAGAUCUGGGAUAAUAUGACAUGGAUGAACUGGUCUAAGGAGAUCAGCAAUUACACA
AACACCAUCUAUAAGCUGCUGGAGGACUCACAGAAUCAGCAGGAAUCAAACAACAAAUCCCUGCU
GGCACUGGACUGAUAA (SEQ ID NO: 206)
X1632_FJ817370_MD39_L14G8 (amino acid, dna, rna)
MDWTWILFLVAAATRVHSSNNLWVTVYYGVPVWEDADTTLFCASDAKAYSTESHNVWATHACVPTDP
NPQEIYLENVTEDFNMWENNMVEQMQEDIISLWDESLKPCVKLTPLCVTLTCTNVTNVTDSVGTNSRLK
GYKEELKNCSFNTTTEIRDKKKQEYALFYKLDIVPINDNSNNSNGYRLINCNVSTIKQACPKVSFDPIPIHYCA
PAGFAILKCRDKEFNGTGTCRNVSTVQCTHGIKPVVSTQLLLNGSLAEGDIIIRSENITDNAKTIIVHLNKTVSI
TCTRPNNNTVKSIRIGPGQALYYTGAIIGDTRQAHCNINGSEWYEMIQNVKNKLNETFKKNITFAPSSGGD
LEITTHSFNCRGEFFYCNTSELFNSSHLFNGSTLSTNGTITLPCRIKQIVRMWQRVGQAMYAPPIAGNITCR
SNITGLLLTRDGGTNKDTNEAETFRPGGGDMRDNWRSELYKYKVVKIKPLGVAPTRCRRRVVGSHSGSGG
SGSGGHAAIGLGTVSLGFLGTAGSTMGAASITLTVQVRQLLSGIVQQQSNLLRAPEPQQHLLQDTHWGIK
QLQARVLAVEHYLKDQQJLGIWGCSGKLICCTNVPWNSSWSNKSYSDIWDNLTWINWSREISNYTQQIYT
LLEESQNQQEKNNQSLLALD** (SEQ ID NO: 210)
atggactggacttggattctgttcctggtcgccgccgctacacgggtgcattcatcaaataacctgtgggtcactgtctactatggggtg
cccgtgtgggaggacgccgataccacactgttctgcgcatccgacgcaaaggcatactccaccgagtctcacaacgtgtgggcaacc
cacgcatgcgtgccaacagacccaaacccccaggagatctatctggagaacgtgacagaggacttcaacatgtgggagaacaatat
ggtggagcagatgcaggaggacatcatcagcctgtgggatgagtccctgaagccttgcgtgaagctgaccccactgtgcgtgacact
gacctgtacaaatgtgaccaacgtgacagactctgtgggcacaaatagccgcctgaagggctacaaggaggagctgaagaactgta
gcttcaataccacaaccgagatcagggataagaagaagcaggagtacgccctgttttataagctggacatcgtgccaatcaatgata
acagcaacaattccaacggctacagactgatcaattgcaacgtgtccaccatcaagcaggcctgtccaaaggtgtctttcgaccctat
cccaatccactattgcgcaccagcaggattcgcaatcctgaagtgtcgcgataaggagtttaatggcaccggcacatgcaggaacgt
gagcaccgtgcagtgtacacacggcatcaagcccgtggtgtctacccagctgctgctgaatggcagcctggccgagggcgacatcat
catcagatccgagaacatcaccgataatgccaagacaatcatcgtgcacctgaacaagaccgtgagcatcacctgcacacgcccca
acaataacacagtgaagtccatcaggatcggccctggccaggccctgtactataccggagcaatcatcggcgacacaaggcaggcc
cactgtaatatcaacggctccgagtggtacgagatgatccagaatgtgaagaacaagctgaatgagacattcaagaagaacatcac
atttgcccccagctccggcggcgatctggagatcacaacccactcttttaactgccgcggcgagttcttttattgtaacaccagcgagc
tgttcaattctagccacctgtttaacggctctaccctgagcacaaacggcaccatcacactgccttgcaggatcaagcagatcgtgcg
catgtggcagagggtgggacaggcaatgtacgcccctcccatcgccggcaatatcacctgtagatctaacatcaccggcctgctgct
gacacgggacggcggaaccaacaaggatacaaatgaggcagagacattcagacccggcggcggcgacatgagagataactggcg
gagcgagctgtacaagtataaggtggtgaagatcaagccactgggagtggcaccaaccaggtgcaggagacgggtggtgggcagc
cactccggctctggcggcagcggctccggcggccacgcagcaatcggcctgggcaccgtgagcctgggctttctgggaaccgcagg
ctccacaatgggagcagcctctatcaccctgacagtgcaggtgagacagctgctgagcggcatcgtgcagcagcagtccaacctgct
gagggcaccagagcctcagcagcacctgctgcaggacacccactggggcatcaagcagctgcaggcccgcgtgctggcagtggag
cactacctgaaggatcagcagatcctgggcatctggggctgttccggcaagctgatctgctgtaccaacgtgccctggaattcctcttg
gtctaataagtcttatagcgacatctgggataacctgacatggatcaattggtccagggagatctctaactacacccagcagatctat
acactgctggaagaaagtcagaatcagcaggagaagaataatcagagcctgctggcactggattgataa (SEQ ID NO: 208)
AUGGACUGGACUUGGAUUCUGUUCCUGGUCGCCGCCGCUACACGGGUGCAUUCAUCAAAUAAC
CUGUGGGUCACUGUCUACUAUGGGGUGCCCGUGUGGGAGGACGCCGAUACCACACUGUUCUGC
GCAUCCGACGCAAAGGCAUACUCCACCGAGUCUCACAACGUGUGGGCAACCCACGCAUGCGUGCC
AACAGACCCAAACCCCCAGGAGAUCUAUCUGGAGAACGUGACAGAGGACUUCAACAUGUGGGAG
AACAAUAUGGUGGAGCAGAUGCAGGAGGACAUCAUCAGCCUGUGGGAUGAGUCCCUGAAGCCU
UGCGUGAAGCUGACCCCACUGUGCGUGACACUGACCUGUACAAAUGUGACCAACGUGACAGACU
CUGUGGGCACAAAUAGCCGCCUGAAGGGCUACAAGGAGGAGCUGAAGAACUGUAGCUUCAAUA
CCACAACCGAGAUCAGGGAUAAGAAGAAGCAGGAGUACGCCCUGUUUUAUAAGCUGGACAUCGU
GCCAAUCAAUGAUAACAGCAACAAUUCCAACGGCUACAGACUGAUCAAUUGCAACGUGUCCACCA
UCAAGCAGGCCUGUCCAAAGGUGUCUUUCGACCCUAUCCCAAUCCACUAUUGCGCACCAGCAGG
AUUCGCAAUCCUGAAGUGUCGCGAUAAGGAGUUUAAUGGCACCGGCACAUGCAGGAACGUGAG
CACCGUGCAGUGUACACACGGCAUCAAGCCCGUGGUGUCUACCCAGCUGCUGCUGAAUGGCAGC
CUGGCCGAGGGCGACAUCAUCAUCAGAUCCGAGAACAUCACCGAUAAUGCCAAGACAAUCAUCG
UGCACCUGAACAAGACCGUGAGCAUCACCUGCACACGCCCCAACAAUAACACAGUGAAGUCCAUC
AGGAUCGGCCCUGGCCAGGCCCUGUACUAUACCGGAGCAAUCAUCGGCGACACAAGGCAGGCCC
ACUGUAAUAUCAACGGCUCCGAGUGGUACGAGAUGAUCCAGAAUGUGAAGAACAAGCUGAAUG
AGACAUUCAAGAAGAACAUCACAUUUGCCCCCAGCUCCGGCGGCGAUCUGGAGAUCACAACCCAC
UCUUUUAACUGCCGCGGCGAGUUCUUUUAUUGUAACACCAGCGAGCUGUUCAAUUCUAGCCAC
CUGUUUAACGGCUCUACCCUGAGCACAAACGGCACCAUCACACUGCCUUGCAGGAUCAAGCAGA
UCGUGCGCAUGUGGCAGAGGGUGGGACAGGCAAUGUACGCCCCUCCCAUCGCCGGCAAUAUCAC
CUGUAGAUCUAACAUCACCGGCCUGCUGCUGACACGGGACGGCGGAACCAACAAGGAUACAAAU
GAGGCAGAGACAUUCAGACCCGGCGGCGGCGACAUGAGAGAUAACUGGCGGAGCGAGCUGUAC
AAGUAUAAGGUGGUGAAGAUCAAGCCACUGGGAGUGGCACCAACCAGGUGCAGGAGACGGGUG
GUGGGCAGCCACUCCGGCUCUGGCGGCAGCGGCUCCGGCGGCCACGCAGCAAUCGGCCUGGGCA
CCGUGAGCCUGGGCUUUCUGGGAACCGCAGGCUCCACAAUGGGAGCAGCCUCUAUCACCCUGAC
AGUGCAGGUGAGACAGCUGCUGAGCGGCAUCGUGCAGCAGCAGUCCAACCUGCUGAGGGCACCA
GAGCCUCAGCAGCACCUGCUGCAGGACACCCACUGGGGCAUCAAGCAGCUGCAGGCCCGCGUGC
UGGCAGUGGAGCACUACCUGAAGGAUCAGCAGAUCCUGGGCAUCUGGGGCUGUUCCGGCAAGC
UGAUCUGCUGUACCAACGUGCCCUGGAAUUCCUCUUGGUCUAAUAAGUCUUAUAGCGACAUCU
GGGAUAACCUGACAUGGAUCAAUUGGUCCAGGGAGAUCUCUAACUACACCCAGCAGAUCUAUAC
ACUGCUGGAAGAAAGUCAGAAUCAGCAGGAGAAGAAUAAUCAGAGCCUGCUGGCACUGGAUUG
AUAA (SEQ ID NO: 209)
CNE8_HM215427_MD39_L14G8 (amino acid, dna, rna)
MDWTWILFLVAAATRVHSSDNLWVTVYYGVPVWRDADTTLFCASDAKAYDTEVHNVWATHACVPTDP
NPQEIHLENVTENFNMWKNKMAEQMQEDVISLWDESLKPCVQLTPLCVTLNCTNANLNATVNASTTIG
NITDEVRNCSFNTTTELRDKKQNVYALFYKLDIVPINNNSEYRLINCNTSVIKQACPKVSFDPIPIHYCAPAGY
AILRCNDKNFNGTGPCKNVSSVQCTHGIKPVVSTQLLLNGSLAEDEIIIRSENLTDNVKTIIVHLNKSVEINCT
RPSNNTVTSVRIGPGQVFYYTGDIIGDIRKAYCEINRTKWHETLKQVATKLREHFNKTIIFQPPSGGDIEITM
HHFNCRGEFFYCNTTKLFNSTWGENTTMEGHNDTIVLPCRIKQIVNMWQGVGQAMYAPPIRGSINCVS
NITGILLTRDGGTNMSNETFRPGGGNIKDNWRSELYKYKVVEIEPLGIAPTKCKRRVVGSHSGSGGSGSGG
HAAVGIGAMSFGFLGAAGSTMGAASITLTVQARQLLSGIVQQQSNLLRAPEPQQHLLQDTHWGIKQLQA
RVLAVEHYLKDQKFLGLWGCSGKIICCTAVPWNSTWSNRSYEEIWDNMTWINWSREISNYTSQIYEILTES
QNQQDRNNKSLLELD** (SEQ ID NO: 213)
atggactggacttggattctgttcctggtcgccgctgctacacgagtgcattcatctgataacctgtgggtcaccgtctactatggcgtg
ccagtgtggcgggacgccgataccacactgttctgcgccagcgacgccaaggcctacgataccgaggtgcacaacgtgtgggcaac
ccacgcatgcgtgccaacagaccctaatccacaggagatccacctggagaacgtgacagagaacttcaacatgtggaagaacaag
atggccgagcagatgcaggaggacgtgatctccctgtgggatgagtctctgaagccctgcgtgcagctgacccctctgtgcgtgaca
ctgaattgtaccaatgccaacctgaatgccaccgtgaatgcctccaccacaatcggcaacatcacagatgaggtgcggaactgttctt
tcaataccacaaccgagctgcgcgacaagaagcagaacgtgtacgccctgttttataagctggatatcgtgcccatcaacaataact
ccgagtatcggctgatcaactgcaatacctctgtgatcaagcaggcctgtcctaaggtgagcttcgaccccatccctatccactactgc
gcaccagcaggatatgcaatcctgcgctgtaatgataagaactttaatggcacaggcccctgcaagaacgtgagctccgtgcagtgt
acccacggcatcaagcctgtggtgtctacacagctgctgctgaacggcagcctggccgaggacgagatcatcatcaggagcgagaa
cctgacagataatgtgaagaccatcatcgtgcacctgaacaagtccgtggagatcaattgcaccaggccatctaataacacagtgac
cagcgtgagaatcggccccggccaggtgttctactatacaggcgacatcatcggcgatatccggaaggcctactgtgagatcaatcg
cacaaagtggcacgagacactgaagcaggtggccaccaagctgagggagcacttcaacaagacaatcatctttcagcccccttccg
gcggcgacatcgagatcaccatgcaccacttcaactgcagaggcgagttcttttactgtaacacaaccaagctgtttaattctacctgg
ggcgagaacacaaccatggagggccacaatgatacaatcgtgctgccttgcagaatcaagcagatcgtgaacatgtggcagggagt
gggacaggcaatgtatgccccacccatcaggggcagcatcaactgcgtgagcaatatcacaggcatcctgctgaccagagacggcg
gaacaaacatgtctaatgagacattcaggcctggcggcggcaacatcaaggataattggagaagcgagctgtacaagtataaggtg
gtggagatcgagcctctgggcatcgccccaacaaagtgcaagaggagagtggtgggctctcacagcggctccggcggctctggcag
cggcggccacgccgccgtgggcatcggcgccatgagcttcggctttctgggagcagcaggctccaccatgggagcagcctctatcac
actgaccgtgcaggcaaggcagctgctgagcggcatcgtgcagcagcagtccaacctgctgagggcaccagagccacagcagcac
ctgctgcaggacacccactggggcatcaagcagctgcaggcccgcgtgctggcagtggagcactacctgaaggatcagaagtttct
gggcctgtggggctgttccggcaagatcatctgctgtaccgccgtgccttggaactccacatggtctaatcggagctatgaggagatc
tgggacaacatgacctggatcaattggtcccgcgagatctctaactacacaagccagatctatgagatcctgaccgaatcacagaat
cagcaggacagaaacaacaaatcactgctggaactggactgataa (SEQ ID NO: 211)
AUGGACUGGACUUGGAUUCUGUUCCUGGUCGCCGCUGCUACACGAGUGCAUUCAUCUGAUAAC
CUGUGGGUCACCGUCUACUAUGGCGUGCCAGUGUGGCGGGACGCCGAUACCACACUGUUCUGC
GCCAGCGACGCCAAGGCCUACGAUACCGAGGUGCACAACGUGUGGGCAACCCACGCAUGCGUGC
CAACAGACCCUAAUCCACAGGAGAUCCACCUGGAGAACGUGACAGAGAACUUCAACAUGUGGAA
GAACAAGAUGGCCGAGCAGAUGCAGGAGGACGUGAUCUCCCUGUGGGAUGAGUCUCUGAAGCC
CUGCGUGCAGCUGACCCCUCUGUGCGUGACACUGAAUUGUACCAAUGCCAACCUGAAUGCCACC
GUGAAUGCCUCCACCACAAUCGGCAACAUCACAGAUGAGGUGCGGAACUGUUCUUUCAAUACCA
CAACCGAGCUGCGCGACAAGAAGCAGAACGUGUACGCCCUGUUUUAUAAGCUGGAUAUCGUGCC
CAUCAACAAUAACUCCGAGUAUCGGCUGAUCAACUGCAAUACCUCUGUGAUCAAGCAGGCCUGU
CCUAAGGUGAGCUUCGACCCCAUCCCUAUCCACUACUGCGCACCAGCAGGAUAUGCAAUCCUGC
GCUGUAAUGAUAAGAACUUUAAUGGCACAGGCCCCUGCAAGAACGUGAGCUCCGUGCAGUGUA
CCCACGGCAUCAAGCCUGUGGUGUCUACACAGCUGCUGCUGAACGGCAGCCUGGCCGAGGACGA
GAUCAUCAUCAGGAGCGAGAACCUGACAGAUAAUGUGAAGACCAUCAUCGUGCACCUGAACAAG
UCCGUGGAGAUCAAUUGCACCAGGCCAUCUAAUAACACAGUGACCAGCGUGAGAAUCGGCCCCG
GCCAGGUGUUCUACUAUACAGGCGACAUCAUCGGCGAUAUCCGGAAGGCCUACUGUGAGAUCA
AUCGCACAAAGUGGCACGAGACACUGAAGCAGGUGGCCACCAAGCUGAGGGAGCACUUCAACAA
GACAAUCAUCUUUCAGCCCCCUUCCGGCGGCGACAUCGAGAUCACCAUGCACCACUUCAACUGCA
GAGGCGAGUUCUUUUACUGUAACACAACCAAGCUGUUUAAUUCUACCUGGGGCGAGAACACAA
CCAUGGAGGGCCACAAUGAUACAAUCGUGCUGCCUUGCAGAAUCAAGCAGAUCGUGAACAUGU
GGCAGGGAGUGGGACAGGCAAUGUAUGCCCCACCCAUCAGGGGCAGCAUCAACUGCGUGAGCAA
UAUCACAGGCAUCCUGCUGACCAGAGACGGCGGAACAAACAUGUCUAAUGAGACAUUCAGGCCU
GGCGGCGGCAACAUCAAGGAUAAUUGGAGAAGCGAGCUGUACAAGUAUAAGGUGGUGGAGAUC
GAGCCUCUGGGCAUCGCCCCAACAAAGUGCAAGAGGAGAGUGGUGGGCUCUCACAGCGGCUCCG
GCGGCUCUGGCAGCGGCGGCCACGCCGCCGUGGGCAUCGGCGCCAUGAGCUUCGGCUUUCUGG
GAGCAGCAGGCUCCACCAUGGGAGCAGCCUCUAUCACACUGACCGUGCAGGCAAGGCAGCUGCU
GAGCGGCAUCGUGCAGCAGCAGUCCAACCUGCUGAGGGCACCAGAGCCACAGCAGCACCUGCUG
CAGGACACCCACUGGGGCAUCAAGCAGCUGCAGGCCCGCGUGCUGGCAGUGGAGCACUACCUGA
AGGAUCAGAAGUUUCUGGGCCUGUGGGGCUGUUCCGGCAAGAUCAUCUGCUGUACCGCCGUGC
CUUGGAACUCCACAUGGUCUAAUCGGAGCUAUGAGGAGAUCUGGGACAACAUGACCUGGAUCA
AUUGGUCCCGCGAGAUCUCUAACUACACAAGCCAGAUCUAUGAGAUCCUGACCGAAUCACAGAA
UCAGCAGGACAGAAACAACAAAUCACUGCUGGAACUGGACUGAUAA (SEQ ID NO: 212)
CNE55_HM215418_MD39_L14G8 (amino acid, dna, rna)
MDWTWILFLVAAATRVHSSDKLWVTVYYGVPVWRDADTTLFCASDAKAHETEVHNVWATHACVPTDP
NPQEIHLVNVTENFNMWKNKMVEQMQEDVISLWDESLKPCVKLTPLCVTLNCTTANTNETKNNTTDDN
IKDEMKNCTFNMTTEIRDKKQRVSALFYKLDIVPIDDSKNNSEYRLINCNTSVIKQACPKVSFDPIPIHYCTPA
GYVILKCNDKNFNGTGPCKNVSSVQCTHGIKPVVSTQLLLNGSLAEEEIIIRSENLTDNAKNIIVHLNKSVEIN
CTRPSNNTVTSVRIGPGQVFYYTGDITGDIRKAYCEIDGTEWNKTLTQVAEKLKEHFNKTIVYQPPSGGDLE
ITMHHFNCRGEFFYCNTTQLFNNSVGNSTIKLPCRIKQIINMWQGVGQAMYAPPISGAINCLSNITGILLTR
DGGGNNRSNETFRPGGGNIKDNWRSELYKYKVVEIEPLGIAPTKCKRRVVGSHSGSGGSGSGGHAAVGIG
AMSFGFLGAAGSTMGAASITLTVQARQLLSGIVQQQSNLLRAPEPQQHMLQDTHWGIKQLQARVLAVE
HYLKDQRFLGLWGCSGKTICCTAVPWNSTWSNKTYEEIWDNMTWTNWSREISNYTNQIYSILTESQSQQ
DKNNKSLLELD** (SEQ ID NO: 216)
atggactggacttggattctgttcctggtcgctgccgctacacgagtgcattcctctgataaactgtgggtgaccgtctactatggagtg
ccagtgtggcgggacgccgataccacactgttctgcgcctctgacgccaaggcccacgagacagaggtgcacaacgtgtgggcaac
ccacgcatgcgtgccaacagatcctaacccacaggagatccacctggtgaatgtgacagagaactttaatatgtggaagaacaaga
tggtggagcagatgcaggaggacgtgatcagcctgtgggatgagtccctgaagccctgcgtgaagctgacccctctgtgcgtgacac
tgaactgtaccacagccaacaccaatgagacaaagaacaataccacagacgataatatcaaggacgagatgaagaactgtacctt
caatatgaccacagagatccgggacaagaagcagcgcgtgagcgccctgttttacaagctggatatcgtgcccatcgacgatagca
agaacaattccgagtatcgcctgatcaactgcaataccagcgtgatcaagcaggcctgtcctaaggtgtccttcgaccccatccctat
ccactactgcaccccagccggctatgtgatcctgaagtgtaacgataagaactttaatggcacaggcccctgcaagaatgtgagctc
cgtgcagtgtacccacggcatcaagcctgtggtgtccacacagctgctgctgaacggctctctggccgaggaggagatcatcatcag
gtctgagaatctgaccgataacgccaagaatatcatcgtgcacctgaacaagagcgtggagatcaattgcacacggccatctaaca
ataccgtgacaagcgtgcgcatcggaccaggacaggtgttctactataccggcgacatcacaggcgatatcagaaaggcctactgtg
agatcgacggcaccgagtggaacaagaccctgacacaggtggccgagaagctgaaggagcactttaataagaccatcgtgtacca
gcccccttccggcggcgatctggagatcacaatgcaccacttcaactgccggggcgagttcttttattgtaataccacacagctgttta
acaattctgtgggcaacagcaccatcaagctgccttgccgcatcaagcagatcatcaatatgtggcagggagtgggacaggcaatgt
acgccccacccatcagcggagccatcaactgtctgtccaatatcaccggcatcctgctgacaagggacggcggcggaaacaatagg
tccaatgagacattcaggcctggcggcggcaacatcaaggataattggagatctgagctgtacaagtataaggtggtggagatcga
gcctctgggcatcgccccaacaaagtgcaagaggagagtggtgggctctcacagcggctccggcggctctggcagcggcggccacg
ccgccgtgggcatcggcgccatgagcttcggctttctgggagcagcaggctccaccatgggagcagcctctatcaccctgacagtgc
aggcccggcagctgctgtctggcatcgtgcagcagcagagcaacctgctgagggcaccagagccacagcagcacatgctgcagga
cacacactggggcatcaagcagctgcaggccagggtgctggcagtggagcactacctgaaggatcagagatttctgggcctgtggg
gctgtagcggcaagaccatctgctgtacagccgtgccttggaactccacctggtctaataagacatatgaggagatctgggacaaca
tgacctggacaaattggtcccgggagatctctaactacaccaatcagatctattccattctgaccgaatcacagtcacagcaggataa
aaataacaaaagtctgctggaactggattgataa (SEQ ID NO: 214)
AUGGACUGGACUUGGAUUCUGUUCCUGGUCGCUGCCGCUACACGAGUGCAUUCCUCUGAUAAA
CUGUGGGUGACCGUCUACUAUGGAGUGCCAGUGUGGCGGGACGCCGAUACCACACUGUUCUGC
GCCUCUGACGCCAAGGCCCACGAGACAGAGGUGCACAACGUGUGGGCAACCCACGCAUGCGUGC
CAACAGAUCCUAACCCACAGGAGAUCCACCUGGUGAAUGUGACAGAGAACUUUAAUAUGUGGAA
GAACAAGAUGGUGGAGCAGAUGCAGGAGGACGUGAUCAGCCUGUGGGAUGAGUCCCUGAAGCC
CUGCGUGAAGCUGACCCCUCUGUGCGUGACACUGAACUGUACCACAGCCAACACCAAUGAGACA
AAGAACAAUACCACAGACGAUAAUAUCAAGGACGAGAUGAAGAACUGUACCUUCAAUAUGACCA
CAGAGAUCCGGGACAAGAAGCAGCGCGUGAGCGCCCUGUUUUACAAGCUGGAUAUCGUGCCCA
UCGACGAUAGCAAGAACAAUUCCGAGUAUCGCCUGAUCAACUGCAAUACCAGCGUGAUCAAGCA
GGCCUGUCCUAAGGUGUCCUUCGACCCCAUCCCUAUCCACUACUGCACCCCAGCCGGCUAUGUG
AUCCUGAAGUGUAACGAUAAGAACUUUAAUGGCACAGGCCCCUGCAAGAAUGUGAGCUCCGUG
CAGUGUACCCACGGCAUCAAGCCUGUGGUGUCCACACAGCUGCUGCUGAACGGCUCUCUGGCCG
AGGAGGAGAUCAUCAUCAGGUCUGAGAAUCUGACCGAUAACGCCAAGAAUAUCAUCGUGCACCU
GAACAAGAGCGUGGAGAUCAAUUGCACACGGCCAUCUAACAAUACCGUGACAAGCGUGCGCAUC
GGACCAGGACAGGUGUUCUACUAUACCGGCGACAUCACAGGCGAUAUCAGAAAGGCCUACUGU
GAGAUCGACGGCACCGAGUGGAACAAGACCCUGACACAGGUGGCCGAGAAGCUGAAGGAGCACU
UUAAUAAGACCAUCGUGUACCAGCCCCCUUCCGGCGGCGAUCUGGAGAUCACAAUGCACCACUU
CAACUGCCGGGGCGAGUUCUUUUAUUGUAAUACCACACAGCUGUUUAACAAUUCUGUGGGCAA
CAGCACCAUCAAGCUGCCUUGCCGCAUCAAGCAGAUCAUCAAUAUGUGGCAGGGAGUGGGACAG
GCAAUGUACGCCCCACCCAUCAGCGGAGCCAUCAACUGUCUGUCCAAUAUCACCGGCAUCCUGC
UGACAAGGGACGGCGGCGGAAACAAUAGGUCCAAUGAGACAUUCAGGCCUGGCGGCGGCAACA
UCAAGGAUAAUUGGAGAUCUGAGCUGUACAAGUAUAAGGUGGUGGAGAUCGAGCCUCUGGGC
AUCGCCCCAACAAAGUGCAAGAGGAGAGUGGUGGGCUCUCACAGCGGCUCCGGCGGCUCUGGCA
GCGGCGGCCACGCCGCCGUGGGCAUCGGCGCCAUGAGCUUCGGCUUUCUGGGAGCAGCAGGCU
CCACCAUGGGAGCAGCCUCUAUCACCCUGACAGUGCAGGCCCGGCAGCUGCUGUCUGGCAUCGU
GCAGCAGCAGAGCAACCUGCUGAGGGCACCAGAGCCACAGCAGCACAUGCUGCAGGACACACACU
GGGGCAUCAAGCAGCUGCAGGCCAGGGUGCUGGCAGUGGAGCACUACCUGAAGGAUCAGAGAU
UUCUGGGCCUGUGGGGCUGUAGCGGCAAGACCAUCUGCUGUACAGCCGUGCCUUGGAACUCCA
CCUGGUCUAAUAAGACAUAUGAGGAGAUCUGGGACAACAUGACCUGGACAAAUUGGUCCCGGG
AGAUCUCUAACUACACCAAUCAGAUCUAUUCCAUUCUGACCGAAUCACAGUCACAGCAGGAUAA
AAAUAACAAAAGUCUGCUGGAACUGGAUUGAUAA (SEQ ID NO: 215)
Other Env sequences
Parts of sequences
Leader sequences
IgE
MDWTWILFLVAAATRVHS (SEQ ID NO: 7)
AD8: atggactggacttggattctgttcctggtcgccgccgctactcgggtgcattct (SEQ ID NO: 2)
001428: atggactggacttggattctgttcctggtggcagcagcaactagagtgcattcc (SEQ ID NO: 3)
Linkers
Link 14 (same as MD3)
GSHSGSGGSGSGGHA (SEQ ID NO: 13)
AD8: tctcacagcggctccggcggctctggcagcggcggccacgcc
001428: ggctcccactctggcagcggcggctccggctctggcggccacgca
GS linkers (same as MD39_TS1)
AD8:&
001428:&
Env parts
AD8 gp120 (AD8_MD64_link14 and AD8_MD64_link14_TS1) amino acid, dna, rna
VENLWVTVYYGVPVWKEATTTLFCASDAKAYDTEVHNVWATHECVPTDPNPQEVVLENVTENFNMWK
NNMVEQMHEDIIELWDQSLKPCVKLTPLCVTLNCTDLRNVTNINNSSEGMRGEIKNCSFNITTSIRDKVKK
DYALFYRLDVVPIDNDNTSYRLINCNTSTITQACPKVSFEPIPIHYCTPAGFAILKCKDKKFNGTGPCKNVSTV
QCTHGIRPVVSTQLLLNGSLAEEEVIIRSSNFTDNAKNIIVQLKESVEINCTRPNNNTVKSIHIGPGRAFYYTG
DIIGDIRQAHCNISRTKWNNTLNQIATKLKEQFGNNKTIVFNQSSGGDPEIVMHSFNCGGEFFYCNSTQLF
NSTWNFNGTWNLTQSNGTEGNDTITLPCRIKQIINMWQEVGKAMYAPPIRGQIRCSSNITGLILTRDGGN
NHNNDTETFRPGGGDMRDNWRSELYKYKVVKIEPLGVAPTKCKRRVVQ (SEQ ID NO: 61)
gtcgaaaacctgtgggtgactgtctattatggagtgcccgtgtggaaggaggccaccacaaccctgttctgcgcctccgacgccaag
gcctacgataccgaggtgcacaacgtgtgggccacccacgagtgcgtgcctacagacccaaacccccaggaggtggtgctggaga
atgtgacagagaacttcaacatgtggaagaacaatatggtggagcagatgcacgaggacatcatcgagctgtgggatcagagcctg
aagccttgcgtgaagctgaccccactgtgcgtgaccctgaattgtacagacctgcggaatgtgacaaacatcaacaatagctccgag
ggcatgagaggcgagatcaagaattgtagcttcaacatcacaacctccatcagggacaaggtgaagaaggattacgccctgttttat
cgcctggatgtggtgcccatcgacaatgataacacctcttaccggctgatcaattgcaacacaagcaccatcacacaggcctgtcca
aaggtgtccttcgagcctatcccaatccactattgcacccccgccggcttcgccatcctgaagtgtaaggacaagaagtttaacggca
caggcccttgcaagaacgtgagcaccgtgcagtgtacacacggcatccggccagtggtgagcacccagctgctgctgaacggctcc
ctggcagaggaggaagtgatcatcagatctagcaatttcacagataatgccaagaacatcatcgtgcagctgaaggagtccgtgga
gatcaactgcacccggcccaacaataacacagtgaagtctatccacatcggccctggcagagccttttactataccggcgacatcatc
ggcgatatcaggcaggcccactgtaacatcagccgcaccaagtggaataacacactgaatcagatcgccaccaagctgaaggagc
agttcggcaataacaagacaatcgtgtttaaccagtcctctggcggcgacccagagatcgtgatgcactcttttaattgcggcggcga
gttcttttactgtaactctacccagctgttcaatagcacatggaacttcaacggcacctggaatctgacacagagcaacggcaccgag
ggcaatgataccatcacactgccctgcaggatcaagcagatcatcaacatgtggcaggaagtgggcaaggccatgtatgcccctccc
atcaggggccagatccgctgtagctccaatatcaccggcctgatcctgacaagggacggcggaaataaccacaataacgataccga
gacattccgccccggcggcggcgacatgagggataactggagatccgagctgtacaagtataaggtggtgaagatcgagccactgg
gagtggcaccaaccaagtgcaagaggagagtggtgcag (SEQ ID NO: 59)
GUCGAAAACCUGUGGGUGACUGUCUAUUAUGGAGUGCCCGUGUGGAAGGAGGCCACCACAACC
CUGUUCUGCGCCUCCGACGCCAAGGCCUACGAUACCGAGGUGCACAACGUGUGGGCCACCCACG
AGUGCGUGCCUACAGACCCAAACCCCCAGGAGGUGGUGCUGGAGAAUGUGACAGAGAACUUCAA
CAUGUGGAAGAACAAUAUGGUGGAGCAGAUGCACGAGGACAUCAUCGAGCUGUGGGAUCAGAG
CCUGAAGCCUUGCGUGAAGCUGACCCCACUGUGCGUGACCCUGAAUUGUACAGACCUGCGGAA
UGUGACAAACAUCAACAAUAGCUCCGAGGGCAUGAGAGGCGAGAUCAAGAAUUGUAGCUUCAAC
AUCACAACCUCCAUCAGGGACAAGGUGAAGAAGGAUUACGCCCUGUUUUAUCGCCUGGAUGUG
GUGCCCAUCGACAAUGAUAACACCUCUUACCGGCUGAUCAAUUGCAACACAAGCACCAUCACACA
GGCCUGUCCAAAGGUGUCCUUCGAGCCUAUCCCAAUCCACUAUUGCACCCCCGCCGGCUUCGCC
AUCCUGAAGUGUAAGGACAAGAAGUUUAACGGCACAGGCCCUUGCAAGAACGUGAGCACCGUGC
AGUGUACACACGGCAUCCGGCCAGUGGUGAGCACCCAGCUGCUGCUGAACGGCUCCCUGGCAGA
GGAGGAAGUGAUCAUCAGAUCUAGCAAUUUCACAGAUAAUGCCAAGAACAUCAUCGUGCAGCU
GAAGGAGUCCGUGGAGAUCAACUGCACCCGGCCCAACAAUAACACAGUGAAGUCUAUCCACAUC
GGCCCUGGCAGAGCCUUUUACUAUACCGGCGACAUCAUCGGCGAUAUCAGGCAGGCCCACUGUA
ACAUCAGCCGCACCAAGUGGAAUAACACACUGAAUCAGAUCGCCACCAAGCUGAAGGAGCAGUU
CGGCAAUAACAAGACAAUCGUGUUUAACCAGUCCUCUGGCGGCGACCCAGAGAUCGUGAUGCAC
UCUUUUAAUUGCGGCGGCGAGUUCUUUUACUGUAACUCUACCCAGCUGUUCAAUAGCACAUGG
AACUUCAACGGCACCUGGAAUCUGACACAGAGCAACGGCACCGAGGGCAAUGAUACCAUCACACU
GCCCUGCAGGAUCAAGCAGAUCAUCAACAUGUGGCAGGAAGUGGGCAAGGCCAUGUAUGCCCCU
CCCAUCAGGGGCCAGAUCCGCUGUAGCUCCAAUAUCACCGGCCUGAUCCUGACAAGGGACGGCG
GAAAUAACCACAAUAACGAUACCGAGACAUUCCGCCCCGGCGGCGGCGACAUGAGGGAUAACUG
GAGAUCCGAGCUGUACAAGUAUAAGGUGGUGAAGAUCGAGCCACUGGGAGUGGCACCAACCAA
GUGCAAGAGGAGAGUGGUGCAG (SEQ ID NO: 60)
AD8gp41 ecto (AD8_MD64_link14 and AD8_MD64_link14_TS1) (amino acid, dna, rna)
AVGTIGAMSLGFLGAAGSTMGAASITLTVQARLLLSGIVQQQNNLLRAPEPQQHLLQLTVWGIKQLQARV
LAVEHYLRDQQLLGIWGCSGKLICCTAVPWNASWSNKTLDMIWNNMTWMEWEREIDNYTGLIYTLIEE
SQNQQEKNEQELLELD (SEQ ID NO: 89)
Gccgtgggcaccatcggcgccatgagcctgggctttctgggagcagcaggctccacaatgggagcagcctctatcaccctgacagt
gcaggccaggctgctgctgtccggcatcgtgcagcagcagaataacctgctgagggcaccagagcctcagcagcacctgctgcagc
tgaccgtgtggggcatcaagcagctgcaggcccgggtgctggcagtggagcactatctgagagatcagcagctgctgggaatctgg
ggatgcagcggcaagctgatctgctgtaccgccgtgccatggaacgcctcctggtctaataagaccctggacatgatctggaataac
atgacatggatggagtgggagcgcgagatcgataactacaccggcctgatctatacactgatcgaggaatcacagaatcagcagga
gaaaaacgaacaggaactgctggaactggat (SEQ ID NO: 87)
GCCGUGGGCACCAUCGGCGCCAUGAGCCUGGGCUUUCUGGGAGCAGCAGGCUCCACAAUGGGA
GCAGCCUCUAUCACCCUGACAGUGCAGGCCAGGCUGCUGCUGUCCGGCAUCGUGCAGCAGCAGA
AUAACCUGCUGAGGGCACCAGAGCCUCAGCAGCACCUGCUGCAGCUGACCGUGUGGGGCAUCAA
GCAGCUGCAGGCCCGGGUGCUGGCAGUGGAGCACUAUCUGAGAGAUCAGCAGCUGCUGGGAAU
CUGGGGAUGCAGCGGCAAGCUGAUCUGCUGUACCGCCGUGCCAUGGAACGCCUCCUGGUCUAA
UAAGACCCUGGACAUGAUCUGGAAUAACAUGACAUGGAUGGAGUGGGAGCGCGAGAUCGAUAA
CUACACCGGCCUGAUCUAUACACUGAUCGAGGAAUCACAGAAUCAGCAGGAGAAAAACGAACAG
GAACUGCUGGAACUGGAU (SEQ ID NO: 88)
001428 gp120 (001428_MD39_link14, 001428_MD39_link14_TS1) (amino acid, dna, rna)
VENLWVTVYYGVPVWKEARTTLFCASDAKAYETEVHNVWATHACVPTDPNPQEMVLGNVTENFNMW
KNDMVDQMHEDVISLWAQSLKPCVKLTPLCVTLECTQVNATQGNTTQVNVTQVNGDEMKNCSFNTTT
EIRDKKQKAYALFYRLDLVPLERENRGDSNSASKYILINCNTSAITQACPKVNFDPIPIHYCTPAGYAILKCNN
KTFNGTGSCNNVSTVQCTHGIKPVVSTQLLLNGSLAEEEIIIRSENLTDNVKTIIVHLDQSVEIVCTRPNNNT
VKSIRIGPGQTFYYTGDIIGNIREAHCNISEKKWHEMLRRVSEKLAEHFPNKTIKFTSSSGGDLEITTHSFNCR
GEFFYCNTSGLFNSTYMPNGTYMPNGTNNSNSTIILPCRIKQIINMWQEVGRAMYAPPIAGNITCNSNIT
GLLLVRDGGKNNNTEIFRPGGGDMRDNWRSELYKYKVVEIKPLGVAPTRCKRRVV (SEQ ID NO: 64)
gtcgaaaacctgtgggtgaccgtgtattatggagtgcccgtgtggaaggaggcccggaccacactgttctgcgcctccgacgccaag
gcctacgagacagaggtgcacaacgtgtgggccacacacgcctgcgtgcctaccgatccaaatccccaggagatggtgctgggcaa
cgtgaccgagaactttaatatgtggaagaacgacatggtggatcagatgcacgaggacgtgatctctctgtgggcccagagcctgaa
gccttgcgtgaagctgaccccactgtgcgtgacactggagtgtacccaggtgaacgccacacagggcaataccacacaggtgaacg
tgacccaagtgaatggcgacgagatgaagaactgttccttcaataccacaaccgagatccgggataagaagcagaaggcctacgcc
ctgttttatagactggacctggtgcctctggagcgggagaacagaggcgattctaatagcgcctccaagtatatcctgatcaactgca
atacatctgccatcacccaggcctgtcctaaagtgaatttcgatcctatcccaatccactactgcaccccagccggctatgccatcctg
aagtgtaacaacaagaccttcaacggcaccggctcctgcaacaacgtgagcacagtgcagtgtacccacggcatcaagccagtggt
gagcacccagctgctgctgaacggctccctggcagaggaggagatcatcatcaggtccgagaacctgacagacaatgtgaagacc
atcatcgtgcacctggatcagtccgtggagatcgtgtgcacacggccaaacaataacaccgtgaagtctatcagaatcggccccggc
cagacattctactataccggcgacatcatcggcaatatccgggaggcccactgtaacatctctgagaagaagtggcacgagatgctg
cggagagtgagcgagaagctggccgagcacttccccaataagacaatcaagtttaccagctcctctggcggcgatctggagatcac
aacccacagcttcaactgcagaggcgagttcttttactgtaacaccagcggcctgtttaattccacatacatgcccaacggcacctata
tgcctaatggcacaaataactctaacagcaccatcatcctgccatgccggatcaagcagatcatcaatatgtggcaggaagtgggca
gagccatgtatgcccctcccatcgccggcaacatcacatgtaacagcaatatcaccggcctgctgctggtgagggacggcggcaag
aataacaatacagagatcttccgccccggcggcggcgacatgagggataactggcgctccgagctgtacaagtataaggtggtgga
gatcaagccactgggagtggcaccaaccaggtgcaagaggcgcgtggtg (SEQ ID NO: 62)
GUCGAAAACCUGUGGGUGACCGUGUAUUAUGGAGUGCCCGUGUGGAAGGAGGCCCGGACCACA
CUGUUCUGCGCCUCCGACGCCAAGGCCUACGAGACAGAGGUGCACAACGUGUGGGCCACACACG
CCUGCGUGCCUACCGAUCCAAAUCCCCAGGAGAUGGUGCUGGGCAACGUGACCGAGAACUUUAA
UAUGUGGAAGAACGACAUGGUGGAUCAGAUGCACGAGGACGUGAUCUCUCUGUGGGCCCAGAG
CCUGAAGCCUUGCGUGAAGCUGACCCCACUGUGCGUGACACUGGAGUGUACCCAGGUGAACGCC
ACACAGGGCAAUACCACACAGGUGAACGUGACCCAAGUGAAUGGCGACGAGAUGAAGAACUGUU
CCUUCAAUACCACAACCGAGAUCCGGGAUAAGAAGCAGAAGGCCUACGCCCUGUUUUAUAGACU
GGACCUGGUGCCUCUGGAGCGGGAGAACAGAGGCGAUUCUAAUAGCGCCUCCAAGUAUAUCCU
GAUCAACUGCAAUACAUCUGCCAUCACCCAGGCCUGUCCUAAAGUGAAUUUCGAUCCUAUCCCA
AUCCACUACUGCACCCCAGCCGGCUAUGCCAUCCUGAAGUGUAACAACAAGACCUUCAACGGCAC
CGGCUCCUGCAACAACGUGAGCACAGUGCAGUGUACCCACGGCAUCAAGCCAGUGGUGAGCACC
CAGCUGCUGCUGAACGGCUCCCUGGCAGAGGAGGAGAUCAUCAUCAGGUCCGAGAACCUGACAG
ACAAUGUGAAGACCAUCAUCGUGCACCUGGAUCAGUCCGUGGAGAUCGUGUGCACACGGCCAAA
CAAUAACACCGUGAAGUCUAUCAGAAUCGGCCCCGGCCAGACAUUCUACUAUACCGGCGACAUC
AUCGGCAAUAUCCGGGAGGCCCACUGUAACAUCUCUGAGAAGAAGUGGCACGAGAUGCUGCGG
AGAGUGAGCGAGAAGCUGGCCGAGCACUUCCCCAAUAAGACAAUCAAGUUUACCAGCUCCUCUG
GCGGCGAUCUGGAGAUCACAACCCACAGCUUCAACUGCAGAGGCGAGUUCUUUUACUGUAACAC
CAGCGGCCUGUUUAAUUCCACAUACAUGCCCAACGGCACCUAUAUGCCUAAUGGCACAAAUAAC
UCUAACAGCACCAUCAUCCUGCCAUGCCGGAUCAAGCAGAUCAUCAAUAUGUGGCAGGAAGUGG
GCAGAGCCAUGUAUGCCCCUCCCAUCGCCGGCAACAUCACAUGUAACAGCAAUAUCACCGGCCUG
CUGCUGGUGAGGGACGGCGGCAAGAAUAACAAUACAGAGAUCUUCCGCCCCGGCGGCGGCGACA
UGAGGGAUAACUGGCGCUCCGAGCUGUACAAGUAUAAGGUGGUGGAGAUCAAGCCACUGGGAG
UGGCACCAACCAGGUGCAAGAGGCGCGUGGUG (SEQ ID NO: 63)
001428 gp41 ecto (001428_MD39_link14_{,} 001428_MD39_link14_TS1) (amino acid, dna, rna)
AVGLGAVSLGFLGAAGSTMGAASITLTVQARQLLSGIVQQQSNLLQAPEPQQHLLQDTHWGIKQLQTRV
LAIEHYLKDQQLLGIWGCSGKLICCTAVPWNSSWSNKSLTDIWDNMTWMQWDREVSNYTGIIYRLLEDS
QNQQERNEQDLLALD (SEQ ID NO: 92)
Gcagtgggcctgggagccgtgagcctgggctttctgggagcagcaggctctaccatgggagcagccagcatcacactgaccgtgca
ggcaaggcagctgctgtccggcatcgtgcagcagcagtctaacctgctgcaggcaccagagcctcagcagcacctgctgcaggaca
cacactggggcatcaagcagctgcagacccgcgtgctggccatcgagcactacctgaaggatcagcagctgctgggcatctggggc
tgctctggcaagctgatctgctgtacagccgtgccttggaacagctcctggagcaataagtccctgacagacatctgggataatatga
cctggatgcagtgggatagggaggtgagcaactacaccggcatcatctatcgcctgctggaagactcacagaatcagcaggaaagg
aatgaacaggatctgctggcactggac (SEQ ID NO: 90)
GCAGUGGGCCUGGGAGCCGUGAGCCUGGGCUUUCUGGGAGCAGCAGGCUCUACCAUGGGAGCA
GCCAGCAUCACACUGACCGUGCAGGCAAGGCAGCUGCUGUCCGGCAUCGUGCAGCAGCAGUCUA
ACCUGCUGCAGGCACCAGAGCCUCAGCAGCACCUGCUGCAGGACACACACUGGGGCAUCAAGCA
GCUGCAGACCCGCGUGCUGGCCAUCGAGCACUACCUGAAGGAUCAGCAGCUGCUGGGCAUCUG
GGGCUGCUCUGGCAAGCUGAUCUGCUGUACAGCCGUGCCUUGGAACAGCUCCUGGAGCAAUAA
GUCCCUGACAGACAUCUGGGAUAAUAUGACCUGGAUGCAGUGGGAUAGGGAGGUGAGCAACUA
CACCGGCAUCAUCUAUCGCCUGCUGGAAGACUCACAGAAUCAGCAGGAAAGGAAUGAACAGGAU
CUGCUGGCACUGGAC (SEQ ID NO: 91)
Full length sequences
AD8_MD64_link14 (amino acid, dna, rna)
MDWTWILFLVAAATRVHSVENLWVTVYYGVPVWKEATTTLFCASDAKAYDTEVHNVWATHECVPTDP
NPQEVVLENVTENFNMWKNNMVEQMHEDIIELWDQSLKPCVKLTPLCVTLNCTDLRNVTNINNSSEGM
RGEIKNCSFNITTSIRDKVKKDYALFYRLDVVPIDNDNTSYRLINCNTSTITQACPKVSFEPIPIHYCTPAGFAIL
KCKDKKFNGTGPCKNVSTVQCTHGIRPVVSTQLLLNGSLAEEEVIIRSSNFTDNAKNIIVQLKESVEINCTRP
NNNTVKSIHIGPGRAFYYTGDIIGDIRQAHCNISRTKWNNTLNQIATKLKEQFGNNKTIVFNQSSGGDPEIV
MHSFNCGGEFFYCNSTQLFNSTWNFNGTWNLTQSNGTEGNDTITLPCRIKQIINMWQEVGKAMYAPPI
RGQIRCSSNITGLILTRDGGNNHNNDTETFRPGGGDMRDNWRSELYKYKVVKIEPLGVAPTKCKRVVQS
HSGSGGSGSGGHAAVGTIGAMSLGFLGAAGSTMGAASITLTVQARLLLSGIVQQQNNLLRAPEPQQHLL
QLTVWGIKQLQARVLAVEHYLRDQQLLGIWGCSGKLICCTAVPWNASWSNKTLDMIWNNMTWMEW
EREIDNYTGLIYTLIEESQNQQEKNEQELLELD** (SEQ ID NO: 219)
atggactggacttggattctgttcctggtcgccgccgctactcgggtgcattctgtcgaaaacctgtgggtgactgtctattatggagtg
cccgtgtggaaggaggccaccacaaccctgttctgcgcctccgacgccaaggcctacgataccgaggtgcacaacgtgtgggccac
ccacgagtgcgtgcctacagacccaaacccccaggaggtggtgctggagaatgtgacagagaacttcaacatgtggaagaacaat
atggtggagcagatgcacgaggacatcatcgagctgtgggatcagagcctgaagccttgcgtgaagctgaccccactgtgcgtgac
cctgaattgtacagacctgcggaatgtgacaaacatcaacaatagctccgagggcatgagaggcgagatcaagaattgtagcttca
acatcacaacctccatcagggacaaggtgaagaaggattacgccctgttttatcgcctggatgtggtgcccatcgacaatgataaca
cctcttaccggctgatcaattgcaacacaagcaccatcacacaggcctgtccaaaggtgtccttcgagcctatcccaatccactattgc
acccccgccggcttcgccatcctgaagtgtaaggacaagaagtttaacggcacaggcccttgcaagaacgtgagcaccgtgcagtgt
acacacggcatccggccagtggtgagcacccagctgctgctgaacggctccctggcagaggaggaagtgatcatcagatctagcaa
tttcacagataatgccaagaacatcatcgtgcagctgaaggagtccgtggagatcaactgcacccggcccaacaataacacagtga
agtctatccacatcggccctggcagagccttttactataccggcgacatcatcggcgatatcaggcaggcccactgtaacatcagccg
caccaagtggaataacacactgaatcagatcgccaccaagctgaaggagcagttcggcaataacaagacaatcgtgtttaaccagt
cctctggcggcgacccagagatcgtgatgcactcttttaattgcggcggcgagttcttttactgtaactctacccagctgttcaatagca
catggaacttcaacggcacctggaatctgacacagagcaacggcaccgagggcaatgataccatcacactgccctgcaggatcaag
cagatcatcaacatgtggcaggaagtgggcaaggccatgtatgcccctcccatcaggggccagatccgctgtagctccaatatcacc
ggcctgatcctgacaagggacggcggaaataaccacaataacgataccgagacattccgccccggcggcggcgacatgagggata
actggagatccgagctgtacaagtataaggtggtgaagatcgagccactgggagtggcaccaaccaagtgcaagaggagagtggt
gcagtctcacagcggctccggcggctctggcagcggcggccacgccgccgtgggcaccatcggcgccatgagcctgggctttctggg
agcagcaggctccacaatgggagcagcctctatcaccctgacagtgcaggccaggctgctgctgtccggcatcgtgcagcagcaga
ataacctgctgagggcaccagagcctcagcagcacctgctgcagctgaccgtgtggggcatcaagcagctgcaggcccgggtgctg
gcagtggagcactatctgagagatcagcagctgctgggaatctggggatgcagcggcaagctgatctgctgtaccgccgtgccatgg
aacgcctcctggtctaataagaccctggacatgatctggaataacatgacatggatggagtgggagcgcgagatcgataactacacc
ggcctgatctatacactgatcgaggaatcacagaatcagcaggagaaaaacgaacaggaactgctggaactggattgataa
(SEQ ID NO: 217)
AUGGACUGGACUUGGAUUCUGUUCCUGGUCGCCGCCGCUACUCGGGUGCAUUCUGUCGAAAAC
CUGUGGGUGACUGUCUAUUAUGGAGUGCCCGUGUGGAAGGAGGCCACCACAACCCUGUUCUGC
GCCUCCGACGCCAAGGCCUACGAUACCGAGGUGCACAACGUGUGGGCCACCCACGAGUGCGUGC
CUACAGACCCAAACCCCCAGGAGGUGGUGCUGGAGAAUGUGACAGAGAACUUCAACAUGUGGAA
GAACAAUAUGGUGGAGCAGAUGCACGAGGACAUCAUCGAGCUGUGGGAUCAGAGCCUGAAGCC
UUGCGUGAAGCUGACCCCACUGUGCGUGACCCUGAAUUGUACAGACCUGCGGAAUGUGACAAA
CAUCAACAAUAGCUCCGAGGGCAUGAGAGGCGAGAUCAAGAAUUGUAGCUUCAACAUCACAACC
UCCAUCAGGGACAAGGUGAAGAAGGAUUACGCCCUGUUUUAUCGCCUGGAUGUGGUGCCCAUC
GACAAUGAUAACACCUCUUACCGGCUGAUCAAUUGCAACACAAGCACCAUCACACAGGCCUGUCC
AAAGGUGUCCUUCGAGCCUAUCCCAAUCCACUAUUGCACCCCCGCCGGCUUCGCCAUCCUGAAG
UGUAAGGACAAGAAGUUUAACGGCACAGGCCCUUGCAAGAACGUGAGCACCGUGCAGUGUACAC
ACGGCAUCCGGCCAGUGGUGAGCACCCAGCUGCUGCUGAACGGCUCCCUGGCAGAGGAGGAAG
UGAUCAUCAGAUCUAGCAAUUUCACAGAUAAUGCCAAGAACAUCAUCGUGCAGCUGAAGGAGUC
CGUGGAGAUCAACUGCACCCGGCCCAACAAUAACACAGUGAAGUCUAUCCACAUCGGCCCUGGC
AGAGCCUUUUACUAUACCGGCGACAUCAUCGGCGAUAUCAGGCAGGCCCACUGUAACAUCAGCC
GCACCAAGUGGAAUAACACACUGAAUCAGAUCGCCACCAAGCUGAAGGAGCAGUUCGGCAAUAA
CAAGACAAUCGUGUUUAACCAGUCCUCUGGCGGCGACCCAGAGAUCGUGAUGCACUCUUUUAA
UUGCGGCGGCGAGUUCUUUUACUGUAACUCUACCCAGCUGUUCAAUAGCACAUGGAACUUCAA
CGGCACCUGGAAUCUGACACAGAGCAACGGCACCGAGGGCAAUGAUACCAUCACACUGCCCUGCA
GGAUCAAGCAGAUCAUCAACAUGUGGCAGGAAGUGGGCAAGGCCAUGUAUGCCCCUCCCAUCAG
GGGCCAGAUCCGCUGUAGCUCCAAUAUCACCGGCCUGAUCCUGACAAGGGACGGCGGAAAUAAC
CACAAUAACGAUACCGAGACAUUCCGCCCCGGCGGCGGCGACAUGAGGGAUAACUGGAGAUCCG
AGCUGUACAAGUAUAAGGUGGUGAAGAUCGAGCCACUGGGAGUGGCACCAACCAAGUGCAAGA
GGAGAGUGGUGCAGUCUCACAGCGGCUCCGGCGGCUCUGGCAGCGGCGGCCACGCCGCCGUGG
GCACCAUCGGCGCCAUGAGCCUGGGCUUUCUGGGAGCAGCAGGCUCCACAAUGGGAGCAGCCUC
UAUCACCCUGACAGUGCAGGCCAGGCUGCUGCUGUCCGGCAUCGUGCAGCAGCAGAAUAACCUG
CUGAGGGCACCAGAGCCUCAGCAGCACCUGCUGCAGCUGACCGUGUGGGGCAUCAAGCAGCUGC
AGGCCCGGGUGCUGGCAGUGGAGCACUAUCUGAGAGAUCAGCAGCUGCUGGGAAUCUGGGGAU
GCAGCGGCAAGCUGAUCUGCUGUACCGCCGUGCCAUGGAACGCCUCCUGGUCUAAUAAGACCCU
GGACAUGAUCUGGAAUAACAUGACAUGGAUGGAGUGGGAGCGCGAGAUCGAUAACUACACCGG
CCUGAUCUAUACACUGAUCGAGGAAUCACAGAAUCAGCAGGAGAAAAACGAACAGGAACUGCUG
GAACUGGAUUGAUAA (SEQ ID NO: 218)
AD8_MD64_link14_TS1 (amino acid, dna, rna)
Repeat 1 optimized for human
Repeat 2 optimized for human/mouse
Repeat 3 optimized for mouse to prevent recombination and large repeats on the nucleic
acid level
MDWTWILFLVAAATRVHSVENLWVTVYYGVPVWKEATTTLFCASDAKAYDTEVHNVWATHECVPTDP
NPQEVVLENVTENFNMWKNNMVEQMHEDIIELWDQSLKPCVKLTPLCVTLNCTDLRNVTNINNSSEGM
RGEIKNCSFNITTSIRDKVKKDYALFYRLDVVPIDNDNTSYRLINCNTSTITQACPKVSFEPIPIHYCTPAGFAIL
KCKDKKFNGTGPCKNVSTVQCTHGIRPVVSTQLLLNGSLAEEEVIIRSSNFTDNAKNIIVQLKESVEINCTRP
NNNTVKSIHIGPGRAFYYTGDIIGDIRQAHCNISRTKWNNTLNQIATKLKEQFGNNKTIVFNQSSGGDPEIV
MHSFNCGGEFFYCNSTQLFNSTWNFNGTWNLTQSNGTEGNDTITLPCRIKQIINMWQEVGKAMYAPPI
RGQIRCSSNITGLILTRDGGNNHNNDTETFRPGGGDMRDNWRSELYKYKVVKIEPLGVAPTKCKRRVVQS
HSGSGGSGSGGHAAVGTIGAMSLGFLGAAGSTMGAASITLTVQARLLLSGIVQQQNNLLRAPEPQQHLL
QLTVWGIKQLQARVLAVEHYLRDQQLLGIWGCSGKLICCTAVPWNASWSNKTLDMIWNNMTWMEW
EREIDNYTGLIYTLIEESQNQQEKNEQELLELDGGVENLWVTVYYGVPVWKEATTTLFCASDAKAYDTEVH
NVWATHECVPTDPNPQEVVLENVTENFNMWKNNMVEQMHEDIIELWDQSLKPCVKLTPLCVTLNCTD
LRNVTNINNSSEGMRGEIKNCSFNITTSIRDKVKKDYALFYRLDVVPIDNDNTSYRLINCNTSTITQACPKVSF
EPIPIHYCTPAGFAILKCKDKKFNGTGPCKNVSTVQCTHGIRPVVSTQLLLNGSLAEEEVIIRSSNFTDNAKNII
VQLKESVEINCTRPNNNTVKSIHIGPGRAFYYTGDIIGDIRQAHCNISRTKWNNTLNQIATKLKEQFGNNKT
IVFNQSSGGDPEIVMHSFNCGGEFFYCNSTQLFNSTWNFNGTWNLTQSNGTEGNDTITLPCRIKQIINM
WQEVGKAMYAPPIRGQIRCSSNITGLILTRDGGNNHNNDTETFRPGGGDMRDNWRSELYKYKVVKIEPL
GVAPTKCKRRVVQSHSGSGGSGSGGHAAVGTIGAMSLGFLGAAGSTMGAASITLTVQARLLLSGIVQQQ
NNLLRAPEPQQHLLQLTVWGIKQLQARVLAVEHYLRDQQLLGIWGCSGKLICCTAVPWNASWSNKTLD
MIWNNMTWMEWEREIDNYTGLIYTLIEESQNQQEKNEQELLELDGGVENLWVTVYYGVPVWKEATTTL
FCASDAKAYDTEVHNVWATHECVPTDPNPQEVVLENVTENFNMWKNNMVEQMHEDIIELWDQSLKP
CVKLTPLCVTLNCTDLRNVTNINNSSEGMRGEIKNCSFNITTSIRDKVKKDYALFYRLDVVPIDNDNTSYRLI
NCNTSTITQACPKVSFEPIPIHYCTPAGFAILKCKDKKFNGTGPCKNVSTVQCTHGIRPVVSTQLLLNGSLAE
EEVIIRSSNFTDNAKNIIVQLKESVEINCTRPNNNTVKSIHIGPGRAFYYTGDIIGDIRQAHCNISRTKWNNTL
NQIATKLKEQFGNNKTIVFNQSSGGDPEIVMHSFNCGGEFFYCNSTQLFNSTWNFNGTWNLTQSNGTE
GNDTITLPCRIKQIINMWQEVGKAMYAPPIRGQIRCSSNITGLILTRDGGNNHNNDTETFRPGGGDMRD
NWRSELYKYKVVKIEPLGVAPTKCKRRVVQSHSGSGGSGSGGHAAVGTIGAMSLGFLGAAGSTMGAASIT
LTVQARLLLSGIVQQQNNLLRAPEPQQHLLQLTVWGIKQLQARVLAVEHYLRDQQLLGIWGCSGKLICCT
AVPWNASWSNKTLDMIWNNMTWMEWEREIDNYTGLIYTLIEESQNQQEKNEQELLELD** (SEQ ID
NO: 222)
atggactggacttggattctgttcctggtcgccgccgctactcgggtgcattctgtcgaaaacctgtgggtgactgtctattatggagtg
cccgtgtggaaggaggccaccacaaccctgttctgcgcctccgacgccaaggcctacgataccgaggtgcacaacgtgtgggccac
ccacgagtgcgtgcctacagacccaaacccccaggaggtggtgctggagaatgtgacagagaacttcaacatgtggaagaacaat
atggtggagcagatgcacgaggacatcatcgagctgtgggatcagagcctgaagccttgcgtgaagctgaccccactgtgcgtgac
cctgaattgtacagacctgcggaatgtgacaaacatcaacaatagctccgagggcatgagaggcgagatcaagaattgtagcttca
acatcacaacctccatcagggacaaggtgaagaaggattacgccctgttttatcgcctggatgtggtgcccatcgacaatgataaca
cctcttaccggctgatcaattgcaacacaagcaccatcacacaggcctgtccaaaggtgtccttcgagcctatcccaatccactattgc
acccccgccggcttcgccatcctgaagtgtaaggacaagaagtttaacggcacaggcccttgcaagaacgtgagcaccgtgcagtgt
acacacggcatccggccagtggtgagcacccagctgctgctgaacggctccctggcagaggaggaagtgatcatcagatctagcaa
tttcacagataatgccaagaacatcatcgtgcagctgaaggagtccgtggagatcaactgcacccggcccaacaataacacagtga
agtctatccacatcggccctggcagagccttttactataccggcgacatcatcggcgatatcaggcaggcccactgtaacatcagccg
caccaagtggaataacacactgaatcagatcgccaccaagctgaaggagcagttcggcaataacaagacaatcgtgtttaaccagt
cctctggcggcgacccagagatcgtgatgcactcttttaattgcggcggcgagttcttttactgtaactctacccagctgttcaatagca
catggaacttcaacggcacctggaatctgacacagagcaacggcaccgagggcaatgataccatcacactgccctgcaggatcaag
cagatcatcaacatgtggcaggaagtgggcaaggccatgtatgcccctcccatcaggggccagatccgctgtagctccaatatcacc
ggcctgatcctgacaagggacggcggaaataaccacaataacgataccgagacattccgccccggcggcggcgacatgagggata
actggagatccgagctgtacaagtataaggtggtgaagatcgagccactgggagtggcaccaaccaagtgcaagaggagagtggt
gcagtctcacagcggctccggcggctctggcagcggcggccacgccgccgtgggcaccatcggcgccatgagcctgggctttctggg
agcagcaggctccacaatgggagcagcctctatcaccctgacagtgcaggccaggctgctgctgtccggcatcgtgcagcagcaga
ataacctgctgagggcaccagagcctcagcagcacctgctgcagctgaccgtgtggggcatcaagcagctgcaggcccgggtgctg
gcagtggagcactatctgagagatcagcagctgctgggaatctggggatgcagcggcaagctgatctgctgtaccgccgtgccatgg
aacgcctcctggtctaataagaccctggacatgatctggaataacatgacatggatggagtgggagcgcgagatcgataactacacc
ggcctgatctatacactgatcgaggaatcacagaatcagcaggagaaaaacgaacaggaactgctggaactggatgtcgaaaatct
ctgggtcaccgtctattatggggtccctgtctggaaggaagcaactactactctgttctgtgcctccgatgccaaggcctacgacacag
aggtgcacaacgtgtgggctacacacgagtgcgtgccaaccgatccaaacccccaggaggtggtgctggagaacgtgaccgagaa
cttcaacatgtggaagaacaacatggtggagcagatgcacgaggacatcatcgagctgtgggatcagtccctgaagccttgcgtga
agctgacaccactgtgcgtgacactgaactgtaccgacctgaggaacgtgaccaacatcaacaacagctccgagggaatgagagg
cgagatcaagaactgtagcttcaacatcaccacatccatccgggacaaggtgaagaaggattacgccctgttttaccgcctggatgt
ggtgcccatcgacaacgataacacctcttacaggctgatcaactgcaacaccagcacaatcacccaggcttgtccaaaggtgtccttt
gagcctatcccaatccactactgcacacccgccggcttcgctatcctgaagtgtaaggacaagaagtttaacggaaccggcccttgc
aagaacgtgtctacagtgcagtgtacccacggcatcaggccagtggtgagcacacagctgctgctgaacggcagcctggccgagga
ggaagtgatcatcagatctagcaacttcaccgataacgctaagaacatcatcgtgcagctgaaggagtccgtggagatcaactgcac
aaggcccaacaacaacaccgtgaagtctatccacatcggacctggcagagccttttactacacaggagacatcatcggcgatatccg
gcaggctcactgtaacatcagccgcacaaagtggaacaacaccctgaaccagatcgccacaaagctgaaggagcagttcggcaac
aacaagaccatcgtgtttaaccagtccagcggcggcgaccccgagatcgtgatgcactctttcaactgcggcggagagttcttttact
gtaactctacacagctgttcaacagcacctggaactttaacggaacatggaacctgacccagagcaacggaaccgagggcaacgat
acaatcaccctgccttgccggatcaagcagatcatcaacatgtggcaggaagtgggaaaggccatgtacgctccccctatcagggg
acagatcaggtgtagctccaacatcacaggactgatcctgacccgggacggcggaaacaaccacaacaacgatacagagacattc
aggcctggcggaggcgacatgagggataactggagatccgagctgtacaagtacaaggtggtgaagatcgagccactgggagtgg
ctccaaccaagtgcaagaggagagtggtgcagtctcacagcggcagcggcggcagcggcagcggaggccacgctgctgtgggaac
aatcggagctatgagcctgggatttctgggagctgctggcagcaccatgggagctgcttctatcacactgaccgtgcaggctaggctg
ctgctgtccggaatcgtgcagcagcagaacaacctgctgagggctccagagcctcagcagcacctgctgcagctgacagtgtgggg
catcaagcagctgcaggccagggtgctggctgtggagcactacctgagggaccagcagctgctgggcatctggggatgtagcggca
agctgatctgctgtaccgccgtgccatggaacgcttcctggtctaacaagacactggacatgatctggaacaacatgacctggatgg
agtgggagcgcgagatcgataactacacaggcctgatctacaccctgatcgaagaaagtcagaatcagcaggaaaagaacgaaca
ggaactgctggaactggacgtcgagaatctgtgggtcaccgtctattatggagtccccgtctggaaagaggctactactacactgtttt
gtgcaagcgatgccaaggcctacgacacagaggtgcacaacgtgtgggccacacacgagtgcgtgccaaccgatccaaaccccca
ggaggtggtgctggagaatgtgaccgagaatttcaacatgtggaagaacaatatggtggagcagatgcacgaggacatcatcgagc
tgtgggatcagtccctgaagccttgcgtgaagctgacaccactgtgcgtgacactgaactgtaccgacctgaggaatgtgaccaaca
tcaacaatagctccgagggcatgagaggcgagatcaagaattgtagcttcaacatcaccacatccatccgggacaaggtgaagaag
gattacgccctgttttatcgcctggatgtggtgcccatcgacaatgataacacctcttacaggctgatcaattgcaacaccagcacaat
cacccaggcctgtccaaaggtgtcctttgagcctatcccaatccactattgcacacccgccggcttcgccatcctgaagtgtaaggac
aagaagtttaacggcaccggcccttgcaagaacgtgagcacagtgcagtgtacccacggcatcaggccagtggtgagcacacagct
gctgctgaacggctccctggccgaggaggaagtgatcatcagatctagcaatttcaccgataatgccaagaacatcatcgtgcagct
gaaggagtccgtggagatcaactgcacaaggcccaacaataacaccgtgaagtctatccacatcggccctggcagagccttttacta
taccggcgacatcatcggcgatatccggcaggcccactgtaacatcagccgcacaaagtggaataacaccctgaatcagatcgcca
caaagctgaaggagcagttcggcaataacaagaccatcgtgtttaaccagtcctctggcggcgaccccgagatcgtgatgcactcttt
caattgcggcggcgagttcttttactgtaactctacacagctgttcaatagcacctggaacttcaacggcacatggaatctgacccag
agcaacggcaccgagggcaatgatacaatcaccctgccttgccggatcaagcagatcatcaacatgtggcaggaagtgggcaagg
ccatgtatgcccctcccatcaggggacagatcaggtgtagctccaatatcacaggcctgatcctgacccgggacggcggaaataacc
acaataacgatacagagacattcaggcccggcggcggcgacatgagggataactggagatccgagctgtacaagtataaggtggt
gaagatcgagccactgggagtggcaccaaccaagtgcaagaggagagtggtgcagtctcacagcggctccggcggctctggcagc
ggcggccacgcagcagtgggaacaatcggagcaatgagcctgggctttctgggagcagcaggctccaccatgggagcagcctctat
cacactgaccgtgcaggcaaggctgctgctgtccggcatcgtgcagcagcagaataacctgctgagggcaccagagcctcagcagc
acctgctgcagctgacagtgtggggcatcaagcagctgcaggccagggtgctggcagtggagcactatctgagggaccagcagctg
ctgggcatctggggctgtagcggcaagctgatctgctgtaccgccgtgccctggaacgcctcctggtctaataagacactggacatga
tctggaataacatgacctggatggagtgggagcgcgagatcgataactacacaggcctgatctataccctgattgaggagtcacaga
accagcaggaaaagaacgaacaggaactgctggaactggattgataa (SEQ ID NO: 220)
AUGGACUGGACUUGGAUUCUGUUCCUGGUCGCCGCCGCUACUCGGGUGCAUUCUGUCGAAAAC
CUGUGGGUGACUGUCUAUUAUGGAGUGCCCGUGUGGAAGGAGGCCACCACAACCCUGUUCUGC
GCCUCCGACGCCAAGGCCUACGAUACCGAGGUGCACAACGUGUGGGCCACCCACGAGUGCGUGC
CUACAGACCCAAACCCCCAGGAGGUGGUGCUGGAGAAUGUGACAGAGAACUUCAACAUGUGGAA
GAACAAUAUGGUGGAGCAGAUGCACGAGGACAUCAUCGAGCUGUGGGAUCAGAGCCUGAAGCC
UUGCGUGAAGCUGACCCCACUGUGCGUGACCCUGAAUUGUACAGACCUGCGGAAUGUGACAAA
CAUCAACAAUAGCUCCGAGGGCAUGAGAGGCGAGAUCAAGAAUUGUAGCUUCAACAUCACAACC
UCCAUCAGGGACAAGGUGAAGAAGGAUUACGCCCUGUUUUAUCGCCUGGAUGUGGUGCCCAUC
GACAAUGAUAACACCUCUUACCGGCUGAUCAAUUGCAACACAAGCACCAUCACACAGGCCUGUCC
AAAGGUGUCCUUCGAGCCUAUCCCAAUCCACUAUUGCACCCCCGCCGGCUUCGCCAUCCUGAAG
UGUAAGGACAAGAAGUUUAACGGCACAGGCCCUUGCAAGAACGUGAGCACCGUGCAGUGUACAC
ACGGCAUCCGGCCAGUGGUGAGCACCCAGCUGCUGCUGAACGGCUCCCUGGCAGAGGAGGAAG
UGAUCAUCAGAUCUAGCAAUUUCACAGAUAAUGCCAAGAACAUCAUCGUGCAGCUGAAGGAGUC
CGUGGAGAUCAACUGCACCCGGCCCAACAAUAACACAGUGAAGUCUAUCCACAUCGGCCCUGGC
AGAGCCUUUUACUAUACCGGCGACAUCAUCGGCGAUAUCAGGCAGGCCCACUGUAACAUCAGCC
GCACCAAGUGGAAUAACACACUGAAUCAGAUCGCCACCAAGCUGAAGGAGCAGUUCGGCAAUAA
CAAGACAAUCGUGUUUAACCAGUCCUCUGGCGGCGACCCAGAGAUCGUGAUGCACUCUUUUAA
UUGCGGCGGCGAGUUCUUUUACUGUAACUCUACCCAGCUGUUCAAUAGCACAUGGAACUUCAA
CGGCACCUGGAAUCUGACACAGAGCAACGGCACCGAGGGCAAUGAUACCAUCACACUGCCCUGCA
GGAUCAAGCAGAUCAUCAACAUGUGGCAGGAAGUGGGCAAGGCCAUGUAUGCCCCUCCCAUCAG
GGGCCAGAUCCGCUGUAGCUCCAAUAUCACCGGCCUGAUCCUGACAAGGGACGGCGGAAAUAAC
CACAAUAACGAUACCGAGACAUUCCGCCCCGGCGGCGGCGACAUGAGGGAUAACUGGAGAUCCG
AGCUGUACAAGUAUAAGGUGGUGAAGAUCGAGCCACUGGGAGUGGCACCAACCAAGUGCAAGA
GGAGAGUGGUGCAGUCUCACAGCGGCUCCGGCGGCUCUGGCAGCGGCGGCCACGCCGCCGUGG
GCACCAUCGGCGCCAUGAGCCUGGGCUUUCUGGGAGCAGCAGGCUCCACAAUGGGAGCAGCCUC
UAUCACCCUGACAGUGCAGGCCAGGCUGCUGCUGUCCGGCAUCGUGCAGCAGCAGAAUAACCUG
CUGAGGGCACCAGAGCCUCAGCAGCACCUGCUGCAGCUGACCGUGUGGGGCAUCAAGCAGCUGC
AGGCCCGGGUGCUGGCAGUGGAGCACUAUCUGAGAGAUCAGCAGCUGCUGGGAAUCUGGGGAU
GCAGCGGCAAGCUGAUCUGCUGUACCGCCGUGCCAUGGAACGCCUCCUGGUCUAAUAAGACCCU
GGACAUGAUCUGGAAUAACAUGACAUGGAUGGAGUGGGAGCGCGAGAUCGAUAACUACACCGG
CCUGAUCUAUACACUGAUCGAGGAAUCACAGAAUCAGCAGGAGAAAAACGAACAGGAACUGCUG
GAACUGGAUGUCGAAAAUCUCUGGGUCACCGUCUAUUAUGGGGUCCCUGUCUGGAAGGAAGCA
ACUACUACUCUGUUCUGUGCCUCCGAUGCCAAGGCCUACGACACAGAGGUGCACAACGUGUGG
GCUACACACGAGUGCGUGCCAACCGAUCCAAACCCCCAGGAGGUGGUGCUGGAGAACGUGACCG
AGAACUUCAACAUGUGGAAGAACAACAUGGUGGAGCAGAUGCACGAGGACAUCAUCGAGCUGU
GGGAUCAGUCCCUGAAGCCUUGCGUGAAGCUGACACCACUGUGCGUGACACUGAACUGUACCG
ACCUGAGGAACGUGACCAACAUCAACAACAGCUCCGAGGGAAUGAGAGGCGAGAUCAAGAACUG
UAGCUUCAACAUCACCACAUCCAUCCGGGACAAGGUGAAGAAGGAUUACGCCCUGUUUUACCGC
CUGGAUGUGGUGCCCAUCGACAACGAUAACACCUCUUACAGGCUGAUCAACUGCAACACCAGCA
CAAUCACCCAGGCUUGUCCAAAGGUGUCCUUUGAGCCUAUCCCAAUCCACUACUGCACACCCGCC
GGCUUCGCUAUCCUGAAGUGUAAGGACAAGAAGUUUAACGGAACCGGCCCUUGCAAGAACGUG
UCUACAGUGCAGUGUACCCACGGCAUCAGGCCAGUGGUGAGCACACAGCUGCUGCUGAACGGCA
GCCUGGCCGAGGAGGAAGUGAUCAUCAGAUCUAGCAACUUCACCGAUAACGCUAAGAACAUCAU
CGUGCAGCUGAAGGAGUCCGUGGAGAUCAACUGCACAAGGCCCAACAACAACACCGUGAAGUCU
AUCCACAUCGGACCUGGCAGAGCCUUUUACUACACAGGAGACAUCAUCGGCGAUAUCCGGCAGG
CUCACUGUAACAUCAGCCGCACAAAGUGGAACAACACCCUGAACCAGAUCGCCACAAAGCUGAAG
GAGCAGUUCGGCAACAACAAGACCAUCGUGUUUAACCAGUCCAGCGGCGGCGACCCCGAGAUCG
UGAUGCACUCUUUCAACUGCGGCGGAGAGUUCUUUUACUGUAACUCUACACAGCUGUUCAACA
GCACCUGGAACUUUAACGGAACAUGGAACCUGACCCAGAGCAACGGAACCGAGGGCAACGAUAC
AAUCACCCUGCCUUGCCGGAUCAAGCAGAUCAUCAACAUGUGGCAGGAAGUGGGAAAGGCCAUG
UACGCUCCCCCUAUCAGGGGACAGAUCAGGUGUAGCUCCAACAUCACAGGACUGAUCCUGACCC
GGGACGGCGGAAACAACCACAACAACGAUACAGAGACAUUCAGGCCUGGCGGAGGCGACAUGAG
GGAUAACUGGAGAUCCGAGCUGUACAAGUACAAGGUGGUGAAGAUCGAGCCACUGGGAGUGGC
UCCAACCAAGUGCAAGAGGAGAGUGGUGCAGUCUCACAGCGGCAGCGGCGGCAGCGGCAGCGGA
GGCCACGCUGCUGUGGGAACAAUCGGAGCUAUGAGCCUGGGAUUUCUGGGAGCUGCUGGCAGC
ACCAUGGGAGCUGCUUCUAUCACACUGACCGUGCAGGCUAGGCUGCUGCUGUCCGGAAUCGUG
CAGCAGCAGAACAACCUGCUGAGGGCUCCAGAGCCUCAGCAGCACCUGCUGCAGCUGACAGUGU
GGGGCAUCAAGCAGCUGCAGGCCAGGGUGCUGGCUGUGGAGCACUACCUGAGGGACCAGCAGC
UGCUGGGCAUCUGGGGAUGUAGCGGCAAGCUGAUCUGCUGUACCGCCGUGCCAUGGAACGCUU
CCUGGUCUAACAAGACACUGGACAUGAUCUGGAACAACAUGACCUGGAUGGAGUGGGAGCGCG
AGAUCGAUAACUACACAGGCCUGAUCUACACCCUGAUCGAAGAAAGUCAGAAUCAGCAGGAAAA
GAACGAACAGGAACUGCUGGAACUGGACGUCGAGAAUCUGUGGGUCACCGUCUAUUAUGGAGU
CCCCGUCUGGAAAGAGGCUACUACUACACUGUUUUGUGCAAGCGAUGCCAAGGCCUACGACACA
GAGGUGCACAACGUGUGGGCCACACACGAGUGCGUGCCAACCGAUCCAAACCCCCAGGAGGUGG
UGCUGGAGAAUGUGACCGAGAAUUUCAACAUGUGGAAGAACAAUAUGGUGGAGCAGAUGCACG
AGGACAUCAUCGAGCUGUGGGAUCAGUCCCUGAAGCCUUGCGUGAAGCUGACACCACUGUGCG
UGACACUGAACUGUACCGACCUGAGGAAUGUGACCAACAUCAACAAUAGCUCCGAGGGCAUGAG
AGGCGAGAUCAAGAAUUGUAGCUUCAACAUCACCACAUCCAUCCGGGACAAGGUGAAGAAGGAU
UACGCCCUGUUUUAUCGCCUGGAUGUGGUGCCCAUCGACAAUGAUAACACCUCUUACAGGCUG
AUCAAUUGCAACACCAGCACAAUCACCCAGGCCUGUCCAAAGGUGUCCUUUGAGCCUAUCCCAA
UCCACUAUUGCACACCCGCCGGCUUCGCCAUCCUGAAGUGUAAGGACAAGAAGUUUAACGGCAC
CGGCCCUUGCAAGAACGUGAGCACAGUGCAGUGUACCCACGGCAUCAGGCCAGUGGUGAGCACA
CAGCUGCUGCUGAACGGCUCCCUGGCCGAGGAGGAAGUGAUCAUCAGAUCUAGCAAUUUCACC
GAUAAUGCCAAGAACAUCAUCGUGCAGCUGAAGGAGUCCGUGGAGAUCAACUGCACAAGGCCCA
ACAAUAACACCGUGAAGUCUAUCCACAUCGGCCCUGGCAGAGCCUUUUACUAUACCGGCGACAU
CAUCGGCGAUAUCCGGCAGGCCCACUGUAACAUCAGCCGCACAAAGUGGAAUAACACCCUGAAU
CAGAUCGCCACAAAGCUGAAGGAGCAGUUCGGCAAUAACAAGACCAUCGUGUUUAACCAGUCCU
CUGGCGGCGACCCCGAGAUCGUGAUGCACUCUUUCAAUUGCGGCGGCGAGUUCUUUUACUGUA
ACUCUACACAGCUGUUCAAUAGCACCUGGAACUUCAACGGCACAUGGAAUCUGACCCAGAGCAA
CGGCACCGAGGGCAAUGAUACAAUCACCCUGCCUUGCCGGAUCAAGCAGAUCAUCAACAUGUGG
CAGGAAGUGGGCAAGGCCAUGUAUGCCCCUCCCAUCAGGGGACAGAUCAGGUGUAGCUCCAAUA
UCACAGGCCUGAUCCUGACCCGGGACGGCGGAAAUAACCACAAUAACGAUACAGAGACAUUCAG
GCCCGGCGGCGGCGACAUGAGGGAUAACUGGAGAUCCGAGCUGUACAAGUAUAAGGUGGUGAA
GAUCGAGCCACUGGGAGUGGCACCAACCAAGUGCAAGAGGAGAGUGGUGCAGUCUCACAGCGG
CUCCGGCGGCUCUGGCAGCGGCGGCCACGCAGCAGUGGGAACAAUCGGAGCAAUGAGCCUGGGC
UUUCUGGGAGCAGCAGGCUCCACCAUGGGAGCAGCCUCUAUCACACUGACCGUGCAGGCAAGGC
UGCUGCUGUCCGGCAUCGUGCAGCAGCAGAAUAACCUGCUGAGGGCACCAGAGCCUCAGCAGCA
CCUGCUGCAGCUGACAGUGUGGGGCAUCAAGCAGCUGCAGGCCAGGGUGCUGGCAGUGGAGCA
CUAUCUGAGGGACCAGCAGCUGCUGGGCAUCUGGGGCUGUAGCGGCAAGCUGAUCUGCUGUAC
CGCCGUGCCCUGGAACGCCUCCUGGUCUAAUAAGACACUGGACAUGAUCUGGAAUAACAUGACC
UGGAUGGAGUGGGAGCGCGAGAUCGAUAACUACACAGGCCUGAUCUAUACCCUGAUUGAGGAG
UCACAGAACCAGCAGGAAAAGAACGAACAGGAACUGCUGGAACUGGAUUGAUAA (SEQ ID
NO: 221)
001428_MD39_link14 (amino acid, dna, rna)
MDWTWILFLVAAATRVHSVENLWVTVYYGVPVWKEARTTLFCASDAKAYETEVHNVWATHACVPTDP
NPQEMVLGNVTENFNMWKNDMVDQMHEDVISLWAQSLKPCVKLTPLCVTLECTQVNATQGNTTQVN
VTQVNGDEMKNCSFNTTTEIRDKKQKAYALFYRLDLVPLERENRGDSNSASKYILINCNTSAITQACPKVNF
DPIPIHYCTPAGYAILKCNNKTFNGTGSCNNVSTVQCTHGIKPVVSTQLLLNGSLAEEEIIIRSENLTDNVKTII
VHLDQSVEIVCTRPNNNTVKSIRIGPGQTFYYTGDIIGNIREAHCNISEKKWHEMLRRVSEKLAEHFPNKTIK
FTSSSGGDLEITTHSFNCRGEFFYCNTSGLFNSTYMPNGTYMPNGTNNSNSTIILPCRIKQIINMWQEVGR
AMYAPPIAGNITCNSNITGLLLVRDGGKNNNTEIFRPGGGDMRDNWRSELYKYKVVEIKPLGVAPTRCKR
RVVGSHSGSGGSGSGGHAAVGLGAVSLGFLGAAGSTMGAASITLTVQARQLLSGIVQQQSNLLQAPEPQ
QHLLQDTHWGIKQLQTRVLAIEHYLKDQQLLGIWGCSGKLICCTAVPWNSSWSNKSLTDIWDNMTWM
QWDREVSNYTGIIYRLLEDSQNQQERNEQDLLALD** (SEQ ID NO: 225)
atggactggacttggattctgttcctggtggcagcagcaactagagtgcattccgtcgaaaacctgtgggtgaccgtgtattatggagt
gcccgtgtggaaggaggcccggaccacactgttctgcgcctccgacgccaaggcctacgagacagaggtgcacaacgtgtgggcca
cacacgcctgcgtgcctaccgatccaaatccccaggagatggtgctgggcaacgtgaccgagaactttaatatgtggaagaacgac
atggtggatcagatgcacgaggacgtgatctctctgtgggcccagagcctgaagccttgcgtgaagctgaccccactgtgcgtgaca
ctggagtgtacccaggtgaacgccacacagggcaataccacacaggtgaacgtgacccaagtgaatggcgacgagatgaagaact
gttccttcaataccacaaccgagatccgggataagaagcagaaggcctacgccctgttttatagactggacctggtgcctctggagcg
ggagaacagaggcgattctaatagcgcctccaagtatatcctgatcaactgcaatacatctgccatcacccaggcctgtcctaaagtg
aatttcgatcctatcccaatccactactgcaccccagccggctatgccatcctgaagtgtaacaacaagaccttcaacggcaccggct
cctgcaacaacgtgagcacagtgcagtgtacccacggcatcaagccagtggtgagcacccagctgctgctgaacggctccctggca
gaggaggagatcatcatcaggtccgagaacctgacagacaatgtgaagaccatcatcgtgcacctggatcagtccgtggagatcgt
gtgcacacggccaaacaataacaccgtgaagtctatcagaatcggccccggccagacattctactataccggcgacatcatcggca
atatccgggaggcccactgtaacatctctgagaagaagtggcacgagatgctgcggagagtgagcgagaagctggccgagcacttc
cccaataagacaatcaagtttaccagctcctctggcggcgatctggagatcacaacccacagcttcaactgcagaggcgagttctttt
actgtaacaccagcggcctgtttaattccacatacatgcccaacggcacctatatgcctaatggcacaaataactctaacagcaccat
catcctgccatgccggatcaagcagatcatcaatatgtggcaggaagtgggcagagccatgtatgcccctcccatcgccggcaacat
cacatgtaacagcaatatcaccggcctgctgctggtgagggacggcggcaagaataacaatacagagatcttccgccccggcggcg
gcgacatgagggataactggcgctccgagctgtacaagtataaggtggtggagatcaagccactgggagtggcaccaaccaggtgc
aagaggcgcgtggtgggctcccactctggcagcggcggctccggctctggcggccacgcagcagtgggcctgggagccgtgagcct
gggctttctgggagcagcaggctctaccatgggagcagccagcatcacactgaccgtgcaggcaaggcagctgctgtccggcatcgt
gcagcagcagtctaacctgctgcaggcaccagagcctcagcagcacctgctgcaggacacacactggggcatcaagcagctgcag
acccgcgtgctggccatcgagcactacctgaaggatcagcagctgctgggcatctggggctgctctggcaagctgatctgctgtaca
gccgtgccttggaacagctcctggagcaataagtccctgacagacatctgggataatatgacctggatgcagtgggatagggaggtg
agcaactacaccggcatcatctatcgcctgctggaagactcacagaatcagcaggaaaggaatgaacaggatctgctggcactgga
ctgataa (SEQ ID NO: 226)
AUGGACUGGACUUGGAUUCUGUUCCUGGUGGCAGCAGCAACUAGAGUGCAUUCCGUCGAAAAC
CUGUGGGUGACCGUGUAUUAUGGAGUGCCCGUGUGGAAGGAGGCCCGGACCACACUGUUCUG
CGCCUCCGACGCCAAGGCCUACGAGACAGAGGUGCACAACGUGUGGGCCACACACGCCUGCGUG
CCUACCGAUCCAAAUCCCCAGGAGAUGGUGCUGGGCAACGUGACCGAGAACUUUAAUAUGUGG
AAGAACGACAUGGUGGAUCAGAUGCACGAGGACGUGAUCUCUCUGUGGGCCCAGAGCCUGAAG
CCUUGCGUGAAGCUGACCCCACUGUGCGUGACACUGGAGUGUACCCAGGUGAACGCCACACAGG
GCAAUACCACACAGGUGAACGUGACCCAAGUGAAUGGCGACGAGAUGAAGAACUGUUCCUUCAA
UACCACAACCGAGAUCCGGGAUAAGAAGCAGAAGGCCUACGCCCUGUUUUAUAGACUGGACCUG
GUGCCUCUGGAGCGGGAGAACAGAGGCGAUUCUAAUAGCGCCUCCAAGUAUAUCCUGAUCAAC
UGCAAUACAUCUGCCAUCACCCAGGCCUGUCCUAAAGUGAAUUUCGAUCCUAUCCCAAUCCACU
ACUGCACCCCAGCCGGCUAUGCCAUCCUGAAGUGUAACAACAAGACCUUCAACGGCACCGGCUCC
UGCAACAACGUGAGCACAGUGCAGUGUACCCACGGCAUCAAGCCAGUGGUGAGCACCCAGCUGC
UGCUGAACGGCUCCCUGGCAGAGGAGGAGAUCAUCAUCAGGUCCGAGAACCUGACAGACAAUGU
GAAGACCAUCAUCGUGCACCUGGAUCAGUCCGUGGAGAUCGUGUGCACACGGCCAAACAAUAAC
ACCGUGAAGUCUAUCAGAAUCGGCCCCGGCCAGACAUUCUACUAUACCGGCGACAUCAUCGGCA
AUAUCCGGGAGGCCCACUGUAACAUCUCUGAGAAGAAGUGGCACGAGAUGCUGCGGAGAGUGA
GCGAGAAGCUGGCCGAGCACUUCCCCAAUAAGACAAUCAAGUUUACCAGCUCCUCUGGCGGCGA
UCUGGAGAUCACAACCCACAGCUUCAACUGCAGAGGCGAGUUCUUUUACUGUAACACCAGCGGC
CUGUUUAAUUCCACAUACAUGCCCAACGGCACCUAUAUGCCUAAUGGCACAAAUAACUCUAACA
GCACCAUCAUCCUGCCAUGCCGGAUCAAGCAGAUCAUCAAUAUGUGGCAGGAAGUGGGCAGAGC
CAUGUAUGCCCCUCCCAUCGCCGGCAACAUCACAUGUAACAGCAAUAUCACCGGCCUGCUGCUG
GUGAGGGACGGCGGCAAGAAUAACAAUACAGAGAUCUUCCGCCCCGGCGGCGGCGACAUGAGG
GAUAACUGGCGCUCCGAGCUGUACAAGUAUAAGGUGGUGGAGAUCAAGCCACUGGGAGUGGCA
CCAACCAGGUGCAAGAGGCGCGUGGUGGGCUCCCACUCUGGCAGCGGCGGCUCCGGCUCUGGC
GGCCACGCAGCAGUGGGCCUGGGAGCCGUGAGCCUGGGCUUUCUGGGAGCAGCAGGCUCUACC
AUGGGAGCAGCCAGCAUCACACUGACCGUGCAGGCAAGGCAGCUGCUGUCCGGCAUCGUGCAGC
AGCAGUCUAACCUGCUGCAGGCACCAGAGCCUCAGCAGCACCUGCUGCAGGACACACACUGGGG
CAUCAAGCAGCUGCAGACCCGCGUGCUGGCCAUCGAGCACUACCUGAAGGAUCAGCAGCUGCUG
GGCAUCUGGGGCUGCUCUGGCAAGCUGAUCUGCUGUACAGCCGUGCCUUGGAACAGCUCCUGG
AGCAAUAAGUCCCUGACAGACAUCUGGGAUAAUAUGACCUGGAUGCAGUGGGAUAGGGAGGUG
AGCAACUACACCGGCAUCAUCUAUCGCCUGCUGGAAGACUCACAGAAUCAGCAGGAAAGGAAUG
AACAGGAUCUGCUGGCACUGGACUGAUAA (SEQ ID NO: 227)
001428_MD39_link14_TS1 (amino acid, dna, rna)
Repeat 1 optimized for human
Repeat 2 optimized for human/mouse
Repeat 3 optimized for mouse to prevent recombination and large repeats on the nucleic
acid level
MDWTWILFLVAAATRVHSVENLWVTVYYGVPVWKEARTTLFCASDAKAYETEVHNVWATHACVPTDP
NPQEMVLGNVTENFNMWKNDMVDQMHEDVISLWAQSLKPCVKLTPLCVTLECTQVNATQGNTTQVN
VTQVNGDEMKNCSFNTTTEIRDKKQKAYALFYRLDLVPLERENRGDSNSASKYILINCNTSAITQACPKVNF
DPIPIHYCTPAGYAILKCNNKTFNGTGSCNNVSTVQCTHGIKPVVSTQLLLNGSLAEEEIIIRSENLTDNVKTII
VHLDQSVEIVCTRPNNNTVKSIRIGPGQTFYYTGDIIGNIREAHCNISEKKWHEMLRRVSEKLAEHFPNKTIK
FTSSSGGDLEITTHSFNCRGEFFYCNTSGLFNSTYMPNGTYMPNGTNNSNSTIILPCRIKQIINMWQEVGR
AMYAPPIAGNITCNSNITGLLLVRDGGKNNNTEIFRPGGGDMRDNWRSELYKYKVVEIKPLGVAPTRCKR
RVVGSHSGSGGSGSGGHAAVGLGAVSLGFLGAAGSTMGAASITLTVQARQLLSGIVQQQSNLLQAPEPQ
QHLLQDTHWGIKQLQTRVLAIEHYLKDQQLLGIWGCSGKLICCTAVPWNSSWSNKSLTDIWDNMTWM
QWDREVSNYTGIIYRLLEDSQNQQERNEQDLLALDGGVENLWVTVYYGVPVWKEARTTLFCASDAKAYE
TEVHNVWATHACVPTDPNPQEMVLGNVTENFNMWKNDMVDQMHEDVISLWAQSLKPCVKLTPLCV
TLECTQVNATQGNTTQVNVTQVNGDEMKNCSFNTTTEIRDKKQKAYALFYRLDLVPLERENRGDSNSAS
KYILINCNTSAITQACPKVNFDPIPIHYCTPAGYAILKCNNKTFNGTGSCNNVSTVQCTHGIKPVVSTQLLLN
GSLAEEEIIIRSENLTDNVKTIIVHLDQSVEIVCTRPNNNTVKSIRIGPGQTFYYTGDIIGNIREAHCNISEKKW
HEMLRRVSEKLAEHFPNKTIKFTSSSGGDLEITTHSFNCRGEFFYCNTSGLFNSTYMPNGTYMPNGTNNSN
STIILPCRIKQIINMWQEVGRAMYAPPIAGNITCNSNITGLLLVRDGGKNNNTEIFRPGGGDMRDNWRSE
LYKYKVVEIKPLGVAPTRCKRRVVGSHSGSGGSGSGGHAAVGLGAVSLGFLGAAGSTMGAASITLTVQAR
QLLSGIVQQQSNLLQAPEPQQHLLQDTHWGIKQLQTRVLAIEHYLKDQQLLGIWGCSGKLICCTAVPWNS
SWSNKSLTDIWDNMTWMQWDREVSNYTGIIYRLLEDSQNQQERNEQDLLALDGGVENLWVTVYYGVP
VWKEARTTLFCASDAKAYETEVHNVWATHACVPTDPNPQEMVLGNVTENFNMWKNDMVDQMHED
VISLWAQSLKPCVKLTPLCVTLECTQVNATQGNTTQVNVTQVNGDEMKNCSFNTTTEIRDKKQKAYALFY
RLDLVPLERENRGDSNSASKYILINCNTSAITQACPKVNFDPIPIHYCTPAGYAILKCNNKTFNGTGSCNNVS
TVQCTHGIKPVVSTQLLLNGSLAEEEIIIRSENLTDNVKTIIVHLDQSVEIVCTRPNNNTVKSIRIGPGQTFYYT
GDIIGNIREAHCNISEKKWHEMLRRVSEKLAEHFPNKTIKFTSSSGGDLEITTHSFNCRGEFFYCNTSGLFNS
TYMPNGTYMPNGTNNSNSTIILPCRIKQIINMWQEVGRAMYAPPIAGNITCNSNITGLLLVRDGGKNNNT
EIFRPGGGDMRDNWRSELYKYKVVEIKPLGVAPTRCKRRVVGSHSGSGGSGSGGHAAVGLGAVSLGFLG
AAGSTMGAASITLTVQARQLLSGIVQQQSNLLQAPEPQQHLLQDTHWGIKQLQTRVLAIEHYLKDQQLLG
IWGCSGKLICCTAVPWNSSWSNKSLTDIWDNMTWMQWDREVSNYTGIIYRLLEDSQNQQERNEQDLL
ALD** (SEQ ID NO: 228)
ATGGACTGGACTTGGATTCTGTTCCTGGTGGCAGCAGCAACTAGAGTGCATTCCGTCGAAAACCTGT
GGGTGACCGTGTATTATGGAGTGCCCGTGTGGAAGGAGGCCCGGACCACACTGTTCTGCGCCTCCG
ACGCCAAGGCCTACGAGACAGAGGTGCACAACGTGTGGGCCACACACGCCTGCGTGCCTACCGATC
CAAATCCCCAGGAGATGGTGCTGGGCAACGTGACCGAGAACTTTAATATGTGGAAGAACGACATGG
TGGATCAGATGCACGAGGACGTGATCTCTCTGTGGGCCCAGAGCCTGAAGCCTTGCGTGAAGCTGAC
CCCACTGTGCGTGACACTGGAGTGTACCCAGGTGAACGCCACACAGGGCAATACCACACAGGTGAAC
GTGACCCAAGTGAATGGCGACGAGATGAAGAACTGTTCCTTCAATACCACAACCGAGATCCGGGATA
AGAAGCAGAAGGCCTACGCCCTGTTTTATAGACTGGACCTGGTGCCTCTGGAGCGGGAGAACAGAG
GCGATTCTAATAGCGCCTCCAAGTATATCCTGATCAACTGCAATACATCTGCCATCACCCAGGCCTGTC
CTAAAGTGAATTTCGATCCTATCCCAATCCACTACTGCACCCCAGCCGGCTATGCCATCCTGAAGTGTA
ACAACAAGACCTTCAACGGCACCGGCTCCTGCAACAACGTGAGCACAGTGCAGTGTACCCACGGCAT
CAAGCCAGTGGTGAGCACCCAGCTGCTGCTGAACGGCTCCCTGGCAGAGGAGGAGATCATCATCAG
GTCCGAGAACCTGACAGACAATGTGAAGACCATCATCGTGCACCTGGATCAGTCCGTGGAGATCGTG
TGCACACGGCCAAACAATAACACCGTGAAGTCTATCAGAATCGGCCCCGGCCAGACATTCTACTATAC
CGGCGACATCATCGGCAATATCCGGGAGGCCCACTGTAACATCTCTGAGAAGAAGTGGCACGAGAT
GCTGCGGAGAGTGAGCGAGAAGCTGGCCGAGCACTTCCCCAATAAGACAATCAAGTTTACCAGCTCC
TCTGGCGGCGATCTGGAGATCACAACCCACAGCTTCAACTGCAGAGGCGAGTTCTTTTACTGTAACAC
CAGCGGCCTGTTTAATTCCACATACATGCCCAACGGCACCTATATGCCTAATGGCACAAATAACTCTA
ACAGCACCATCATCCTGCCATGCCGGATCAAGCAGATCATCAATATGTGGCAGGAAGTGGGCAGAGC
CATGTATGCCCCTCCCATCGCCGGCAACATCACATGTAACAGCAATATCACCGGCCTGCTGCTGGTGA
GGGACGGCGGCAAGAATAACAATACAGAGATCTTCCGCCCCGGCGGCGGCGACATGAGGGATAACT
GGCGCTCCGAGCTGTACAAGTATAAGGTGGTGGAGATCAAGCCACTGGGAGTGGCACCAACCAGGT
GCAAGAGGCGCGTGGTGGGCTCCCACTCTGGCAGCGGCGGCTCCGGCTCTGGCGGCCACGCAGCA
GTGGGCCTGGGAGCCGTGAGCCTGGGCTTTCTGGGAGCAGCAGGCTCTACCATGGGAGCAGCCAGC
ATCACACTGACCGTGCAGGCAAGGCAGCTGCTGTCCGGCATCGTGCAGCAGCAGTCTAACCTGCTGC
AGGCACCAGAGCCTCAGCAGCACCTGCTGCAGGACACACACTGGGGCATCAAGCAGCTGCAGACCC
GCGTGCTGGCCATCGAGCACTACCTGAAGGATCAGCAGCTGCTGGGCATCTGGGGCTGCTCTGGCA
AGCTGATCTGCTGTACAGCCGTGCCTTGGAACAGCTCCTGGAGCAATAAGTCCCTGACAGACATCTG
GGATAATATGACCTGGATGCAGTGGGATAGGGAGGTGAGCAACTACACCGGCATCATCTATCGCCTG
CTGGAAGACTCACAGAATCAGCAGGAAAGGAATGAACAGGATCTGCTGGCACTGGACGGGGGAGTC
GAGAACCTCTGGGTCACCGTGTATTATGGAGTCCCCGTCTGGAAAGAAGCCCGAACCACCCTGTTTT
GTGCCTCTGATGCTAAAGCCTACGAGACAGAGGTGCACAACGTGTGGGCTACACACGCTTGCGTGCC
AACCGACCCAAACCCCCAGGAGATGGTGCTGGGCAACGTGACCGAGAACTTCAACATGTGGAAGAA
CGACATGGTGGATCAGATGCACGAGGATGTGATCTCTCTGTGGGCCCAGAGCCTGAAGCCTTGCGTG
AAGCTGACCCCACTGTGCGTGACACTGGAGTGTACCCAGGTGAACGCTACACAGGGCAACACCACAC
AGGTGAACGTGACCCAGGTGAACGGAGACGAGATGAAGAACTGTTCCTTCAACACCACAACCGAGA
TCAGGGATAAGAAGCAGAAGGCCTACGCTCTGTTTTACAGACTGGACCTGGTGCCACTGGAGAGGG
AGAACAGAGGCGATTCTAACAGCGCCTCCAAGTACATCCTGATCAACTGCAACACATCTGCCATCACC
CAGGCTTGTCCTAAGGTGAACTTCGACCCTATCCCAATCCACTACTGCACACCAGCCGGCTACGCTAT
CCTGAAGTGTAACAACAAGACCTTCAACGGAACCGGCTCCTGCAACAACGTGTCTACAGTGCAGTGT
ACCCACGGCATCAAGCCCGTGGTGAGCACCCAGCTGCTGCTGAACGGCAGCCTGGCTGAGGAGGAG
ATCATCATCCGGTCCGAGAACCTGACAGACAACGTGAAGACCATCATCGTGCACCTGGATCAGTCCG
TGGAGATCGTGTGCACAAGGCCAAACAACAACACCGTGAAGTCTATCAGAATCGGACCCGGCCAGAC
CTTCTACTACACCGGAGACATCATCGGCAACATCAGGGAGGCCCACTGTAACATCTCTGAGAAGAAG
TGGCACGAGATGCTGAGGAGAGTGAGCGAGAAGCTGGCTGAGCACTTCCCTAACAAGACAATCAAG
TTTACCAGCTCCTCTGGCGGAGATCTGGAGATCACAACCCACAGCTTCAACTGCAGAGGAGAGTTCTT
TTACTGTAACACCAGCGGCCTGTTTAACTCCACATACATGCCCAACGGAACCTACATGCCTAACGGCA
CAAACAACTCTAACAGCACCATCATCCTGCCCTGCAGGATCAAGCAGATCATCAACATGTGGCAGGAA
GTGGGAAGAGCCATGTACGCTCCCCCTATCGCCGGCAACATCACATGTAACAGCAACATCACCGGAC
TGCTGCTGGTGCGGGACGGCGGAAAGAACAACAACACAGAGATCTTCCGCCCTGGCGGAGGCGACA
TGAGGGATAACTGGCGCTCCGAGCTGTACAAGTACAAGGTGGTGGAGATCAAGCCACTGGGAGTGG
CTCCAACCAGGTGCAAGAGGAGGGTGGTGGGCAGCCACTCTGGCAGCGGAGGCTCCGGATCTGGA
GGCCACGCTGCTGTGGGACTGGGAGCCGTGAGCCTGGGATTTCTGGGAGCTGCTGGATCTACCATG
GGAGCTGCTAGCATCACACTGACCGTGCAGGCTAGGCAGCTGCTGTCCGGAATCGTGCAGCAGCAG
TCTAACCTGCTGCAGGCTCCCGAGCCTCAGCAGCACCTGCTGCAGGACACACACTGGGGCATCAAGC
AGCTGCAGACCCGCGTGCTGGCCATCGAGCACTACCTGAAGGATCAGCAGCTGCTGGGCATCTGGG
GATGTTCTGGCAAGCTGATCTGCTGTACAGCTGTGCCATGGAACAGCTCCTGGAGCAACAAGTCCCT
GACAGACATCTGGGATAACATGACCTGGATGCAGTGGGATCGGGAGGTGAGCAACTACACCGGCAT
CATCTACCGCCTGCTGGAAGACTCACAGAATCAGCAGGAACGGAATGAACAGGACCTCCTCGCACTG
GATGGCGGAGTCGAAAACCTGTGGGTCACCGTCTACTATGGAGTGCCAGTGTGGAAAGAGGCTAGG
ACTACCCTGTTCTGTGCCAGCGATGCCAAAGCCTACGAGACAGAGGTGCACAACGTGTGGGCAACAC
ACGCATGCGTGCCAACCGACCCAAATCCCCAGGAGATGGTGCTGGGCAACGTGACCGAGAACTTCAA
TATGTGGAAGAACGACATGGTGGATCAGATGCACGAGGATGTGATCTCTCTGTGGGCCCAGAGCCT
GAAGCCTTGCGTGAAGCTGACCCCACTGTGCGTGACACTGGAGTGTACCCAGGTGAACGCCACACAG
GGCAATACCACACAGGTGAACGTGACCCAAGTGAATGGCGACGAGATGAAGAACTGTTCCTTCAATA
CCACAACCGAGATCAGGGATAAGAAGCAGAAGGCCTACGCCCTGTTTTATAGACTGGACCTGGTGCC
ACTGGAGAGGGAGAACAGAGGCGATTCTAATAGCGCCTCCAAGTATATCCTGATCAACTGCAATACA
TCTGCCATCACCCAGGCCTGTCCTAAAGTGAATTTCGACCCTATCCCAATCCACTACTGCACACCAGCC
GGCTATGCCATCCTGAAGTGTAACAACAAGACCTTCAACGGCACCGGCTCCTGCAACAACGTGAGCA
CAGTGCAGTGTACCCACGGCATCAAGCCCGTGGTGAGCACCCAGCTGCTGCTGAACGGCTCCCTGGC
AGAGGAGGAGATCATCATCCGGTCCGAGAACCTGACAGACAATGTGAAGACCATCATCGTGCACCTG
GATCAGTCCGTGGAGATCGTGTGCACAAGGCCAAACAATAACACCGTGAAGTCTATCAGAATCGGCC
CCGGCCAGACCTTCTACTATACCGGCGACATCATCGGCAATATCAGGGAGGCCCACTGTAACATCTCT
GAGAAGAAGTGGCACGAGATGCTGAGGAGAGTGAGCGAGAAGCTGGCCGAGCACTTCCCTAATAA
GACAATCAAGTTTACCAGCTCCTCTGGCGGCGATCTGGAGATCACAACCCACAGCTTCAACTGCAGA
GGCGAGTTCTTTTACTGTAACACCAGCGGCCTGTTTAATTCCACATACATGCCCAACGGCACCTATAT
GCCTAATGGCACAAATAACTCTAACAGCACCATCATCCTGCCCTGCAGGATCAAGCAGATCATCAATA
TGTGGCAGGAAGTGGGCAGAGCCATGTATGCCCCTCCCATCGCCGGCAACATCACATGTAACAGCAA
TATCACCGGCCTGCTGCTGGTGCGGGACGGCGGCAAGAATAACAATACAGAGATCTTCCGCCCCGGC
GGCGGCGACATGAGGGATAACTGGCGCTCCGAGCTGTACAAGTATAAGGTGGTGGAGATCAAGCCA
CTGGGAGTGGCACCAACCAGGTGCAAGAGGCGCGTGGTGGGCTCCCACTCTGGCAGCGGCGGCTCC
GGCTCTGGCGGCCACGCAGCAGTGGGCCTGGGAGCCGTGTCCCTGGGCTTTCTGGGAGCAGCAGGC
TCTACCATGGGAGCAGCCAGCATCACACTGACCGTGCAGGCAAGGCAGCTGCTGTCCGGCATCGTGC
AGCAGCAGTCTAACCTGCTGCAGGCACCAGAGCCTCAGCAGCACCTGCTGCAGGACACACACTGGG
GCATCAAGCAGCTGCAGACCCGCGTGCTGGCCATCGAGCACTACCTGAAGGATCAGCAGCTGCTGG
GCATCTGGGGCTGTTCTGGCAAGCTGATCTGCTGTACAGCCGTGCCATGGAACAGCTCCTGGAGCAA
TAAGTCCCTGACAGACATCTGGGATAATATGACCTGGATGCAGTGGGATCGGGAGGTGAGCAACTAC
ACCGGCATCATCTATCGCCTGCTGGAGGACTCACAGAATCAGCAGGAGCGGAACGAACAGGATCTG
CTGGCACTGGATTGATAA (SEQ ID NO: 226)
AUGGACUGGACUUGGAUUCUGUUCCUGGUGGCAGCAGCAACUAGAGUGCAUUCCGUCGAAAAC
CUGUGGGUGACCGUGUAUUAUGGAGUGCCCGUGUGGAAGGAGGCCCGGACCACACUGUUCUG
CGCCUCCGACGCCAAGGCCUACGAGACAGAGGUGCACAACGUGUGGGCCACACACGCCUGCGUG
CCUACCGAUCCAAAUCCCCAGGAGAUGGUGCUGGGCAACGUGACCGAGAACUUUAAUAUGUGG
AAGAACGACAUGGUGGAUCAGAUGCACGAGGACGUGAUCUCUCUGUGGGCCCAGAGCCUGAAG
CCUUGCGUGAAGCUGACCCCACUGUGCGUGACACUGGAGUGUACCCAGGUGAACGCCACACAGG
GCAAUACCACACAGGUGAACGUGACCCAAGUGAAUGGCGACGAGAUGAAGAACUGUUCCUUCAA
UACCACAACCGAGAUCCGGGAUAAGAAGCAGAAGGCCUACGCCCUGUUUUAUAGACUGGACCUG
GUGCCUCUGGAGCGGGAGAACAGAGGCGAUUCUAAUAGCGCCUCCAAGUAUAUCCUGAUCAAC
UGCAAUACAUCUGCCAUCACCCAGGCCUGUCCUAAAGUGAAUUUCGAUCCUAUCCCAAUCCACU
ACUGCACCCCAGCCGGCUAUGCCAUCCUGAAGUGUAACAACAAGACCUUCAACGGCACCGGCUCC
UGCAACAACGUGAGCACAGUGCAGUGUACCCACGGCAUCAAGCCAGUGGUGAGCACCCAGCUGC
UGCUGAACGGCUCCCUGGCAGAGGAGGAGAUCAUCAUCAGGUCCGAGAACCUGACAGACAAUGU
GAAGACCAUCAUCGUGCACCUGGAUCAGUCCGUGGAGAUCGUGUGCACACGGCCAAACAAUAAC
ACCGUGAAGUCUAUCAGAAUCGGCCCCGGCCAGACAUUCUACUAUACCGGCGACAUCAUCGGCA
AUAUCCGGGAGGCCCACUGUAACAUCUCUGAGAAGAAGUGGCACGAGAUGCUGCGGAGAGUGA
GCGAGAAGCUGGCCGAGCACUUCCCCAAUAAGACAAUCAAGUUUACCAGCUCCUCUGGCGGCGA
UCUGGAGAUCACAACCCACAGCUUCAACUGCAGAGGCGAGUUCUUUUACUGUAACACCAGCGGC
CUGUUUAAUUCCACAUACAUGCCCAACGGCACCUAUAUGCCUAAUGGCACAAAUAACUCUAACA
GCACCAUCAUCCUGCCAUGCCGGAUCAAGCAGAUCAUCAAUAUGUGGCAGGAAGUGGGCAGAGC
CAUGUAUGCCCCUCCCAUCGCCGGCAACAUCACAUGUAACAGCAAUAUCACCGGCCUGCUGCUG
GUGAGGGACGGCGGCAAGAAUAACAAUACAGAGAUCUUCCGCCCCGGCGGCGGCGACAUGAGG
GAUAACUGGCGCUCCGAGCUGUACAAGUAUAAGGUGGUGGAGAUCAAGCCACUGGGAGUGGCA
CCAACCAGGUGCAAGAGGCGCGUGGUGGGCUCCCACUCUGGCAGCGGCGGCUCCGGCUCUGGC
GGCCACGCAGCAGUGGGCCUGGGAGCCGUGAGCCUGGGCUUUCUGGGAGCAGCAGGCUCUACC
AUGGGAGCAGCCAGCAUCACACUGACCGUGCAGGCAAGGCAGCUGCUGUCCGGCAUCGUGCAGC
AGCAGUCUAACCUGCUGCAGGCACCAGAGCCUCAGCAGCACCUGCUGCAGGACACACACUGGGG
CAUCAAGCAGCUGCAGACCCGCGUGCUGGCCAUCGAGCACUACCUGAAGGAUCAGCAGCUGCUG
GGCAUCUGGGGCUGCUCUGGCAAGCUGAUCUGCUGUACAGCCGUGCCUUGGAACAGCUCCUGG
AGCAAUAAGUCCCUGACAGACAUCUGGGAUAAUAUGACCUGGAUGCAGUGGGAUAGGGAGGUG
AGCAACUACACCGGCAUCAUCUAUCGCCUGCUGGAAGACUCACAGAAUCAGCAGGAAAGGAAUG
AACAGGAUCUGCUGGCACUGGACGGGGGAGUCGAGAACCUCUGGGUCACCGUGUAUUAUGGAG
UCCCCGUCUGGAAAGAAGCCCGAACCACCCUGUUUUGUGCCUCUGAUGCUAAAGCCUACGAGAC
AGAGGUGCACAACGUGUGGGCUACACACGCUUGCGUGCCAACCGACCCAAACCCCCAGGAGAUG
GUGCUGGGCAACGUGACCGAGAACUUCAACAUGUGGAAGAACGACAUGGUGGAUCAGAUGCAC
GAGGAUGUGAUCUCUCUGUGGGCCCAGAGCCUGAAGCCUUGCGUGAAGCUGACCCCACUGUGC
GUGACACUGGAGUGUACCCAGGUGAACGCUACACAGGGCAACACCACACAGGUGAACGUGACCC
AGGUGAACGGAGACGAGAUGAAGAACUGUUCCUUCAACACCACAACCGAGAUCAGGGAUAAGAA
GCAGAAGGCCUACGCUCUGUUUUACAGACUGGACCUGGUGCCACUGGAGAGGGAGAACAGAGG
CGAUUCUAACAGCGCCUCCAAGUACAUCCUGAUCAACUGCAACACAUCUGCCAUCACCCAGGCUU
GUCCUAAGGUGAACUUCGACCCUAUCCCAAUCCACUACUGCACACCAGCCGGCUACGCUAUCCU
GAAGUGUAACAACAAGACCUUCAACGGAACCGGCUCCUGCAACAACGUGUCUACAGUGCAGUGU
ACCCACGGCAUCAAGCCCGUGGUGAGCACCCAGCUGCUGCUGAACGGCAGCCUGGCUGAGGAGG
AGAUCAUCAUCCGGUCCGAGAACCUGACAGACAACGUGAAGACCAUCAUCGUGCACCUGGAUCA
GUCCGUGGAGAUCGUGUGCACAAGGCCAAACAACAACACCGUGAAGUCUAUCAGAAUCGGACCC
GGCCAGACCUUCUACUACACCGGAGACAUCAUCGGCAACAUCAGGGAGGCCCACUGUAACAUCU
CUGAGAAGAAGUGGCACGAGAUGCUGAGGAGAGUGAGCGAGAAGCUGGCUGAGCACUUCCCUA
ACAAGACAAUCAAGUUUACCAGCUCCUCUGGCGGAGAUCUGGAGAUCACAACCCACAGCUUCAA
CUGCAGAGGAGAGUUCUUUUACUGUAACACCAGCGGCCUGUUUAACUCCACAUACAUGCCCAAC
GGAACCUACAUGCCUAACGGCACAAACAACUCUAACAGCACCAUCAUCCUGCCCUGCAGGAUCAA
GCAGAUCAUCAACAUGUGGCAGGAAGUGGGAAGAGCCAUGUACGCUCCCCCUAUCGCCGGCAAC
AUCACAUGUAACAGCAACAUCACCGGACUGCUGCUGGUGCGGGACGGCGGAAAGAACAACAACA
CAGAGAUCUUCCGCCCUGGCGGAGGCGACAUGAGGGAUAACUGGCGCUCCGAGCUGUACAAGU
ACAAGGUGGUGGAGAUCAAGCCACUGGGAGUGGCUCCAACCAGGUGCAAGAGGAGGGUGGUGG
GCAGCCACUCUGGCAGCGGAGGCUCCGGAUCUGGAGGCCACGCUGCUGUGGGACUGGGAGCCG
UGAGCCUGGGAUUUCUGGGAGCUGCUGGAUCUACCAUGGGAGCUGCUAGCAUCACACUGACCG
UGCAGGCUAGGCAGCUGCUGUCCGGAAUCGUGCAGCAGCAGUCUAACCUGCUGCAGGCUCCCG
AGCCUCAGCAGCACCUGCUGCAGGACACACACUGGGGCAUCAAGCAGCUGCAGACCCGCGUGCU
GGCCAUCGAGCACUACCUGAAGGAUCAGCAGCUGCUGGGCAUCUGGGGAUGUUCUGGCAAGCU
GAUCUGCUGUACAGCUGUGCCAUGGAACAGCUCCUGGAGCAACAAGUCCCUGACAGACAUCUGG
GAUAACAUGACCUGGAUGCAGUGGGAUCGGGAGGUGAGCAACUACACCGGCAUCAUCUACCGCC
UGCUGGAAGACUCACAGAAUCAGCAGGAACGGAAUGAACAGGACCUCCUCGCACUGGAUGGCGG
AGUCGAAAACCUGUGGGUCACCGUCUACUAUGGAGUGCCAGUGUGGAAAGAGGCUAGGACUAC
CCUGUUCUGUGCCAGCGAUGCCAAAGCCUACGAGACAGAGGUGCACAACGUGUGGGCAACACAC
GCAUGCGUGCCAACCGACCCAAAUCCCCAGGAGAUGGUGCUGGGCAACGUGACCGAGAACUUCA
AUAUGUGGAAGAACGACAUGGUGGAUCAGAUGCACGAGGAUGUGAUCUCUCUGUGGGCCCAGA
GCCUGAAGCCUUGCGUGAAGCUGACCCCACUGUGCGUGACACUGGAGUGUACCCAGGUGAACG
CCACACAGGGCAAUACCACACAGGUGAACGUGACCCAAGUGAAUGGCGACGAGAUGAAGAACUG
UUCCUUCAAUACCACAACCGAGAUCAGGGAUAAGAAGCAGAAGGCCUACGCCCUGUUUUAUAGA
CUGGACCUGGUGCCACUGGAGAGGGAGAACAGAGGCGAUUCUAAUAGCGCCUCCAAGUAUAUC
CUGAUCAACUGCAAUACAUCUGCCAUCACCCAGGCCUGUCCUAAAGUGAAUUUCGACCCUAUCC
CAAUCCACUACUGCACACCAGCCGGCUAUGCCAUCCUGAAGUGUAACAACAAGACCUUCAACGGC
ACCGGCUCCUGCAACAACGUGAGCACAGUGCAGUGUACCCACGGCAUCAAGCCCGUGGUGAGCA
CCCAGCUGCUGCUGAACGGCUCCCUGGCAGAGGAGGAGAUCAUCAUCCGGUCCGAGAACCUGAC
AGACAAUGUGAAGACCAUCAUCGUGCACCUGGAUCAGUCCGUGGAGAUCGUGUGCACAAGGCC
AAACAAUAACACCGUGAAGUCUAUCAGAAUCGGCCCCGGCCAGACCUUCUACUAUACCGGCGACA
UCAUCGGCAAUAUCAGGGAGGCCCACUGUAACAUCUCUGAGAAGAAGUGGCACGAGAUGCUGA
GGAGAGUGAGCGAGAAGCUGGCCGAGCACUUCCCUAAUAAGACAAUCAAGUUUACCAGCUCCUC
UGGCGGCGAUCUGGAGAUCACAACCCACAGCUUCAACUGCAGAGGCGAGUUCUUUUACUGUAA
CACCAGCGGCCUGUUUAAUUCCACAUACAUGCCCAACGGCACCUAUAUGCCUAAUGGCACAAAU
AACUCUAACAGCACCAUCAUCCUGCCCUGCAGGAUCAAGCAGAUCAUCAAUAUGUGGCAGGAAG
UGGGCAGAGCCAUGUAUGCCCCUCCCAUCGCCGGCAACAUCACAUGUAACAGCAAUAUCACCGG
CCUGCUGCUGGUGCGGGACGGCGGCAAGAAUAACAAUACAGAGAUCUUCCGCCCCGGCGGCGGC
GACAUGAGGGAUAACUGGCGCUCCGAGCUGUACAAGUAUAAGGUGGUGGAGAUCAAGCCACUG
GGAGUGGCACCAACCAGGUGCAAGAGGCGCGUGGUGGGCUCCCACUCUGGCAGCGGCGGCUCC
GGCUCUGGCGGCCACGCAGCAGUGGGCCUGGGAGCCGUGUCCCUGGGCUUUCUGGGAGCAGCA
GGCUCUACCAUGGGAGCAGCCAGCAUCACACUGACCGUGCAGGCAAGGCAGCUGCUGUCCGGCA
UCGUGCAGCAGCAGUCUAACCUGCUGCAGGCACCAGAGCCUCAGCAGCACCUGCUGCAGGACAC
ACACUGGGGCAUCAAGCAGCUGCAGACCCGCGUGCUGGCCAUCGAGCACUACCUGAAGGAUCAG
CAGCUGCUGGGCAUCUGGGGCUGUUCUGGCAAGCUGAUCUGCUGUACAGCCGUGCCAUGGAAC
AGCUCCUGGAGCAAUAAGUCCCUGACAGACAUCUGGGAUAAUAUGACCUGGAUGCAGUGGGAU
CGGGAGGUGAGCAACUACACCGGCAUCAUCUAUCGCCUGCUGGAGGACUCACAGAAUCAGCAGG
AGCGGAACGAACAGGAUCUGCUGGCACUGGAUUGAUAA (SEQ ID NO: 227)

1. A composition comprising an expressible nucleic acid sequence comprising: (i) a first nucleic acid sequence encoding a soluble retroviral trimer or a soluble monomer of a retroviral trimer or a pharmaceutically acceptable salt thereof.

2. The composition of claim 1, wherein the composition further comprises: a regulatory sequence operably linked to the first nucleotide sequence, wherein the first nucleic acid sequence comprises at least about 70% sequence identity to a nucleotide sequence encoding a soluble trimer of human immunodeficiency virus-1 (HIV-1) ENV or a soluble monomer of HIV-1 ENV.

3. (canceled)

4. The composition of claim 1, wherein the expressible nucleic acid sequence comprises the expressible nucleic acid sequence comprising:
(i) one or a combination of nucleic acid sequences chosen from: SEQ ID NO: 53, SEQ ID NO: 54, SEQ ID NO: 56, SEQ ID NO: 57, SEQ ID NO: 59, SEQ ID NO: 60, SEQ ID NO: 62, SEQ ID NO: 63, SEQ ID NO: 78, SEQ ID NO: 79, SEQ ID NO: 81, SEQ ID NO: 82, SEQ ID NO: 84, SEQ ID NO: 85, SEQ ID NO: 87, SEQ ID NO: 88, SEQ ID NO: 90, SEQ ID NO: 91, SEQ ID NO: 106, SEQ ID NO: 107, SEQ ID NO: 109, SEQ ID NO: 110, SEQ ID NO: 112, SEQ ID NO: 113, SEQ ID NO: 115, SEQ ID NO: 116, SEQ ID NO: 118, SEQ ID NO: 119, SEQ ID NO: 121, SEQ ID NO: 122, SEQ ID NO: 124, SEQ ID NO: 125, SEQ ID NO: 127, SEQ ID NO: 128, SEQ ID NO: 130, SEQ ID NO: 131; and/or
(ii) one or a combination of nucleic acid sequences wherein the at least one nucleic acid sequence comprises at least about 70% sequence identity to a sequence identified as including: AD8, CPG9.2, 001428, TR011, X2278, 398F1, 246F3, CE0217, CE1176, 25710, BJOX2000, CH119, X1632, CNE8, CNE55, or 001428; and/or
(iii) one or a combination of nucleic acid sequences that encode an amino acid sequence chosen from: SEQ ID NO: 55, SEQ ID NO: 58, SEQ ID NO: 61, SEQ ID NO: 49, SEQ ID NO: 50, SEQ ID NO: 51, SEQ ID NO: 52, SEQ ID NO: 53, SEQ ID NO: 64, SEQ ID NO: 80, SEQ ID NO: 83, SEQ ID NO: 86, SEQ ID NO: 89, SEQ ID NO: 92, SEQ ID NO: 93, SEQ ID NO: 94, SEQ ID NO: 108, SEQ ID NO: 111, SEQ ID NO: 114, SEQ ID NO: 117, SEQ ID NO: 120, SEQ ID NO: 123, SEQ ID NO: 93, SEQ ID NO: 126, SEQ ID NO: 129, SEQ ID NO: 132; and/or
(iv) one or a combination of nucleic acid sequences that encode at least one amino acid sequences comprising at least about 70% sequence identity to a sequence identified as including: AD8, CPG9.2, 001428, TR011, X2278, 398F1, 246F3, CE0217, CE1176, 25710, BJOX2000, CH119, X1632, CNE8, CNE55, or 001428.

5.-8. (canceled)

9. The composition of claim 1, wherein the expressible nucleic acid sequence further comprises a nucleic acid sequence that encodes a viral antigen comprises at least about 70% sequence identity to SEQ ID NO: 4 or a pharmaceutically acceptable salt thereof.

10. The composition of claim 1, wherein the expressible nucleic acid sequence further comprises at least two non-contiguous nucleic acid sequences comprising at least about 70% sequence identity to a leader sequence and further comprising a sequence encoding a linker positioned between the two non-contiguous nucleic acid sequences.

11-12. (canceled)

13. The composition of claim 1, wherein the first nucleotide sequence encodes at least three HIV antigens, each HIV antigen expressed as a contiguous polypeptide chain that is secreted by a cell upon expression.

14. The composition of claim 1, wherein the first nucleic acid sequence encodes a self-assembling polypeptide comprises at least about 70% sequence identity to SEQ ID NO: 2 or a pharmaceutically acceptable salt thereof.

15. The composition of claim 1 further comprising a nucleic acid molecule that is a DNA plasmid;
wherein the plasmid comprises an expressible nucleic acid sequence comprising at least one nucleic acid or combination thereof that is or encodes a nucleic acid or amino acid comprising at least about 70% sequence identity to an amino acid sequence chosen from: SEQ ID NO: 133 through SEQ ID NO: 153, or a pharmaceutically acceptable salt thereof.

16. A pharmaceutical composition comprising: (i) the composition of claim 1; and (ii) a pharmaceutically acceptable carrier.

17-19. (canceled)

20. A method of vaccinating a subject comprising administering a therapeutically effective amount of the pharmaceutical composition of claim 1 to the subject.

21. The method of claim 20, wherein the administering is accomplished by oral administration, parenteral administration, sublingual administration, transdermal administration, rectal administration, transmucosal administration; topical administration, inhalation, buccal administration, intrapleural administration, intravenous administration, intraarterial administration, intraperitoneal administration, subcutaneous administration intramuscular administration, intranasal administration, intrathecal administration, and intraarticular administration, or combinations thereof.

21. The method of claim 20, wherein the therapeutically effective dose is from about 1 to about 30 micrograms of expressible nucleic acid sequence.

22. The method of claim 20, wherein the method is free of activating any mannose-binding lectin or complement process.

23. The method of claim 20, wherein the subject is a human.

24. (canceled)

25. A method of inducing an immune response in a subject comprising administering to the subject the pharmaceutical composition of claim 16.

26.-33. (canceled)

34. A method of neutralizing one or plurality of viruses in a subject comprising administering to the subject the pharmaceutical composition of claim 16.

35-39. (canceled)

40. A method of stimulating a therapeutically effective antigen-specific immune response against a virus in a mammal infected with the virus comprising administering the pharmaceutical composition of claim 16.

41. The method of claim 40, wherein the method is free of activating any mannose-binding lectin or complement pathway associated with an immune response.

42-43. (canceled)

44. A vaccine comprising an expressible nucleotide sequence comprising:
(i) one or a combination of nucleic acid sequences chosen from: SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, and SEQ ID NO: 72; and/or
(ii) one or a combination of nucleic acid sequences wherein the at least one nucleic acid sequence comprises at least about 70% sequence identity to a sequence chosen from: SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, and SEQ ID NO: 72; and/or
(iii) one or a combination of nucleic acid sequences that encode an amino acid sequence chosen from: SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, and SEQ ID NO: 57; SEQ ID NO: 58, SEQ ID NO: 59; or SEQ ID NO: 60; and/or
(iv) one or a combination of nucleic acid sequences that encode at least one amino acid sequences comprising at least about 70% sequence identity to a sequence chosen from: SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, and SEQ ID NO: 57; SEQ ID NO: 58, SEQ ID NO: 59; or SEQ ID NO: 60.

45. The vaccine of claim 44 further comprising a linker fusing the three expressible noncontiguous nucleic acid sequences.

46. The vaccine of claim 45, wherein the linker is an amino acid sequence comprising at least about 70% sequence identity to SEQ ID NO: 8.

47-57. (canceled)
